SEQUENCE LISTING (110 sequences)

SEQ ID NO: 1 - protein, 1177 aa, Homo sapiens
MAGIFKEFFFSTEDLPEVILTLSLISSIGAFLNRHLEDFPIPVPVILFLLGCSFEVLSFTSSQVQRYANAIQWMSPDLFFRIFTPVVFFTTAFDMDTYMLQKLFWQILLISIPGFLVNYILVLWHLASVNQLLLKPTQWLLFSAILVSSDPMLTAAAIRDLGLSRSLISLINGESLMTSVISLITFTSIMDFDQRLQSKRNHTLAEEIVGGICSYIIASFLFGILSSKLIQFWMSTVFGDDVNHISLIFSILYLIFYICELVGMSGIFTLAIVGLLLNSTSFKAAIEETLLLEFWTFLSRIAFLMVFTFFGLLIPAHTYLYIEFVDIYYSLNIYLTLIVLRFLTLLLISPVLSRVGHEFSWRWIFIMVCSEMKGMPNINMALLLAYSDLYFGSDKEKSQILFHGVLVCLITLVVNRFILPVAVTILGLRDATSTKYKSVCCTFQHFQELTKSAASALKFDKDLANADWNMIEKAITLENPYMLNEEETTEHQKVKCPHCNKEIDEIFNTEAMELANRRLLSAQIASYQRQYRNEILSQSAVQVLVGAAESFGEKKGKCMSLDTIKNYSESQKTVTFARKLLLNWVYNTRKEKEGPSKYFFFRICHTIVFTEEFEHVGYLVILMNIFPFIISWISQLNVIYHSELKHTNYCFLTLYILEALLKIAAMRKDFFSHAWNIFELAITLIGILHVILIEIDTIKYIFNETEVIVFIKVVQFFRILRIFKLIAPKLLQIIDKRMSHQKTFWYGILKGYVQGEADIMTIIDQITSSKQIKQMLLKQVIRNMEHAIKELGYLEYDHPEIAVTVKTKEEINVMLNMATEILKAFGLKGIISKTEGAGINKLIMAKKKEVLDSQSIIRPLTVEEVLYHIPWLDKNKDYINFIQEKAKVVTFDCGNDIFEEGDEPKGIYIIISGMVKLEKSKPGLGIDQMVESKEKDFPIIDTDYMLSGEIIGEINCLTNEPMKYSATCKTVVETCFIPKTHLYDAFEQCSPLIKQKMWLKLGLAITARKIREHLSYEDWNYNMQLKLSNIYVVDIPMSTKTDIYDENLIYVILIHGAVEDCLLRKTYRAPFLIPITCHQIQSIEDFTKVVIIQTPINMKTFRRNIRKFVPKHKSYLTPGLIGSVGTLEEGIQEERNVKEDGAHSAATARSPQPCSLLGTKFNCKESPRINLRKVRKE

SEQ ID NO: 2 - protein, 1909 aa, Homo sapiens
MACCHKVMLLLDTAGGAARHSRVRRAALRLLTYLSCRFGLARVHWAFKFFDSQGARSRPSRVSDFRELGSRSWEDFEEELEARLEDRAHLPGPAPRATHTHGALMETLLDYQWDRPEITSPTKPILRSSGRRLLDVESEAKEAEAALGGLVNAVFLLAPCPHSQRELLQFVSGCEAQAQRLPPTPKQVMEKLLPKRVREVMVARKITFYWVDTTEWSKLWESPDHLGYWTVCELLHHGGGTVLPSESFSWDFAQAGEMLLRSGIKLSSEPHLSPWISMLPTDATLNRLLYNSPEYEASFPRMEGMLFLPVEGKEIQETWTVTLEPLAMHQRHFQKPVRIFLKGSVAQWSLPTSSTLGTDSWMLGSPEESTATQRLLFQQLVSRLTAEELHLVADVDPGEGRPPITGVISPLSASAMILTVCRTKEAEFQRHVLQTAVADSPRDTASLFSDVVDSILNQTHDSLADTASAASPVPEWAQQELGHTTPWSPAVVEKWFPFCNISGASSDLMESFGLLQAASANKEESSKTEGELIHCLAELYQRKSREESTIAHQEDSKKKRGVPRTPVRQKMNTMCRSLKMLNVARLNVKAQKLHPDGSPDVAGEKGIQKIPSGRTVDKLEDRGRTLRSSKPKDFKTEEELLSYIRENYQKTVATGEIMLYACARNMISTVKMFLKSKGTKELEVNCLNQVKSSLLKTSKSLRQNLGKKLDKEDKVRECQLQVFLRLEMCLQCPSINESTDDMEQVVEEVTDLLRMVCLTEDSAYLAEFLEEILRLYIDSIPKTLGNLYNSLGFVIPQKLAGVLPTDFFSDDSMTQENKSPLLSVPFLSSARRSVSGSPESDELQELRTRSAKKRRKNALIRHKSIAEVSQNLRQIEIPKVSKRATKKENSHPAPQQPSQPVKDTVQEVTKVRRNLFNQELLSPSKRSLKRGLPRSHSVSAVDGLEDKLDNFKKNKGYHKLLTKSVAETPVHKQISKRLLHRQIKGRSSDPGPDIGVVEESPEKGDEISLRRSPRIKQLSFSRTHSASFYSVSQPKSRSVQRVHSFQQDKSDQRENSPVQSIRSPKSLLFGAMSEMISPSEKGSARMKKRSRNTLDSEVPAAYQTPKKSHQKSLSFSKTTPRRISHTPQTPLYTPERLQKSPAKMTPTKQAAFKESLKDSSSPGHDSPLDSKITPQKRHTQAGEGTSLETKTPRTPKRQGTQPPGFLPNCTWPHSVNSSPESPSCPAPPTSSTAQPRRECLTPIRDPLRTPPRAAAFMGTPQNQTHQQPHVLRAARAEEPAQKLKDKAIKTPKRPGNSTVTSSPPVTPKKLFTSPLCDVSKKSPFRKSKIECPSPGELDQKEPQMSPSVAASLSCPVPSTPPELSQRATLDTVPPPPPSKVGKRCRKTSDPRRSIVECQPDASATPGVGTADSPAAPTDSRDDQKGLSLSPQSPPERRGYPGPGLRSDWHASSPLLITSDTEHVTLLSEAEHHGIGDLKSNVLSVEEGEGLRTADAEKSSLSHPGIPPSPPSCGPGSPLMPSRDVHCTTDGRQCQASAQLDNLPASAWHSTDSASPQTYEVELEMQASGLPKLRIKKIDPSSSLEAEPLSKEESSLGEESFLPALSMPRASRSLSKPEPTYVSPPCPRLSHSTPGKSRGQTYICQACTPTHGPSSTPSPFQTDGVPWTPSPKHSGKTTPDIIKDWPRRKRAVGCGAGSSSGRGEVGADLPGSLSLLESEGKDHGLELSIHRTPILEDFELEGVCQLPDQSPPRNSMPKAEEASSWGQFGLSSRKRVLLAKEEADRGAKRICDLREDSEVSKSKEGSPSWSAWQLPSTGDEEVFVSGSTPPPSCAVRSCLSASALQALTQSPLLFQGKTPSSQSKDPRDEDVDVLPSTVEDSPFSRAFSRRRPISRTYTRKKLMGTWLEDL

SEQ ID NO: 3 - protein, 309 aa, Homo sp.
MENQNNVTEFILLGLTENLELWKIFSAVFLVMYVATVLENLLIVVTIITSQSLRSPMYFFLTFLSLLDVMFSSVVAPKVIVDTLSKSTTISLKGCLTQLFVEHFFGGVGIILLTVMAYDRYVAICKPLHYTIIMSPRVCCLMVGGAWVGGFMHAMIQLLFMYQIPFCGPNIIDHFICDLFQLLTLACTDTHILGLLVTLNSGMMCVAIFLILIASYTVILCSLKSYSSKGRHKALSTCSSHLTVVVLFFVPCIFLYMRPVVTHPIDKAMAVSDSIITPMLNPLIYTLRNAEVKSAMKKLWMKWEALAGK

SEQ ID NO: 4 - protein, 213 aa, Homo sapiens
MVPEEEPQDREKGLWWFQLKVWSMAVVSILLLSVCFTVSSVVPHNFMYSKTVKRLSKLREYQQYHPSLTCVMEGKDIEDWSCCPTPWTSFQSSCYFISTGMQSWTKSQKNCSVMGADLVVINTREEQDFIIQNLKRNSSYFLGLSDPGGRRHWQWVDQTPYNENVTFWHSGEPNNLDERCAIINFRSSEEWGWNDIHCHVPQKSICKMKKIYI

SEQ ID NO: 5 - protein, 113 aa, Homo sapiens
MASATRLIQRLRNWASGHDLQGKLQLRYQEISKRTQPPPKLPVGPSHKLSNNYYCTRDGRRESVPPSIIMSSQKALVSGKPAESSAVAATEKKAVTPAPPIKRWELSSDQPYL

SEQ ID NO: 6 - protein, 312 aa, Homo sapiens
MSVLNNSEVKLFLLIGIPGLEHAHIWFSIPICLMYLLAIMGNCTILFIIKTEPSLHEPMYYFLAMLAVSDMGLSLSSLPTMLRVFLFNAMGISPNACFAQEFFIHGFTVMESSVLLIMSLDRFLAIHNPLRYSSILTSNRVAKMGLILAIRSILLVIPFPFTLRRLKYCQKNLLSHSYCLHQDTMKLACSDNKTNVIYGFFIALCTMLDLALIVLSYVLILKTILSIASLAERLKALNTCVSHICAVLTFYVPIITLAAMHHFAKHKSPLVVILIADMFLLVPPLMNPIVYCVKTRQIWEKILGKLLNVCGR

SEQ ID NO: 7 - protein, 687 aa, Homo sapiens
MEEFVGLREGSSGNPVTLQELWGPCPRIRRGIRGGLEWLKQKLFRLGEDWYFLMTLGVLMALVSCAMDLAVESVVRAHQWLYREIGDSHLLRYLSWTVYPVALVSFSSGFSQSITPSSGGSGIPEVKTMLAGVVLEDYLDIKNFGAKVVGLSCTLACGSTLFLGKVGPFVHLSVMMAAYLGRVRTTTIGEPENKSKQNEMLVAAAAVGVATVFAAPFSGVLFSIEVMSSHFSVWDYWRGFFAATCGAFMFRLLAVFNSEQETITSLYKTSFRVDVPFDLPEIFFFVALGGLCGILGSAYLFCQRIFFGFIRNNRFSSKLLATSKPVYSALATLVLASITYPPSAGRFLASRLSMKQHLDSLFDNHSWALMTQNSSPPWPEELDPQHLWWEWYHPRFTIFGTLAFFLVMKFWMLILATTIPMPAGYFMPIFVYGAAIGRLFGETLSFIFPEGIVAGGITNPIMPGGYALAGAAAFSGAVTHTISTALLAFEVTGQIVHALPVLMAVLAANAIAQSCQPSFYDGTVIVKKLPYLPRILGRNIGSHRVRVEHFMNHSITTLAKDMPLEEVVKVVTSTDVAKYPLVESTESQILVGIVRRAQLVQALKAEPPSWAPGHQQCLQDILAAGCPTEPVTLKLSPETSLHEAHNLFELLNLHSLFVTSRGRAVGCVSWVEMKKAISNLTNPPAPK

SEQ ID NO: 8 - protein, 68 aa, Homo sapiens
MSGSSSVAAMKKVVQQLRLEAGLNRVKVSQAAADLKQFCLQNAQHDPLLTGVSSSTNPFRPQKVCSFL

SEQ ID NO: 9 - protein, 732 aa, Homo sapiens
MDPSADTWDLFSPLISLWINRFYIYLGFAVSISLWICVQIVIKTQGKNLQEKSVPKAAQDLMTNGYVSLQEKDIFVSGVKIFYGSQTGTAKGFATVLAEAVTSLDLPVAIINLKEYDPDDHLIEEVTSKNVCVFLVATYTDGLPTESAEWFCKWLEEASIDFRFGKTYLKGMRYAVFGLGNSAYASHFNKVGKNVDKWLWMLGAHRVMSRGEGDCDVVKSKHGSIEADFRAWKTKFISQLQALQKGERKKSCGGHCKKGKCESHQHGSEEREEGSHEQDELHHRDTEEEEPFESSSEEEFGGEDHQSLNSIVDVEDLGKIMDHVKKEKREKEQQEEKSGLFRNMGRNEDGERRAMITPALREALTKQGYQLIGSHSGVKLCRWTKSMLRGRGGCYKHTFYGIESHRCMETTPSLACANKCVFCWRHHTNPVGTEWRWKMDQPEMILKEAIENHQNMIKQFKGVPGVKAERFEEGMTVKHCALSLVGEPIMYPEINRFLKLLHQCKISSFLVTNAQFPAEIRNLEPVTQLYVSVDASTKDSLKKIDRPLFKDFWQRFLDSLKALAVKQQRTVYRLTLVKAWNVDELQAYAQLVSLGNPDFIEVKGVTYCGESSASSLTMAHVPWHEEVVQFVHELVDLIPEYEIACEHEHSNCLLIAHRKFKIGGEWWTWIDYNRFQELIQEYEDSGGSKTFSAKDYMARTPHWALFGASERGFDPKDTRHQRKNKSKAISGC

SEQ ID NO: 10 - protein, 218 aa, Homo sapiens
MEAEGCRYQFRVALLGDAAVGKTSLLRSYVAGAPGAPEPEPEPEPTVGAECYRRALQLRAGPRVKLQLWDTAGHERFRCITRSFYRNVVGVLLVFDVTNRKSFEHIQDWHQEVMATQGPDKVIFLLVGHKSDLQSTRCVSAQEAEELAASLGMAFVETSVKNNCNVDLAFDTLADAIQQALQQGDIKLEEGWGGVRLIHKTQIPRSPSRKQHSGPCQC

SEQ ID NO: 11 - protein, 774 aa, Homo sapiens
MEAEQRPAAGASEGATPGLEAVPPVAPPPATAASGPIPKSGPEPKRRHLGTLLQPTVNKFSLRVFGSHKAVEIEQERVKSAGAWIIHPYSDFRFYWDLIMLLLMVGNLIVLPVGITFFKEENSPPWIVFNVLSDTFFLLDLVLNFRTGIVVEEGAEILLAPRAIRTRYLRTWFLVDLISSIPVDYIFLVVELEPRLDAEVYKTARALRIVRFTKILSLLRLLRLSRLIRYIHQWEEIFHMTYDLASAVVRIFNLIGMMLLLCHWDGCLQFLVPMLQDFPPDCWVSINHMVNHSWGRQYSHALFKAMSHMLCIGYGQQAPVGMPDVWLTMLSMIVGATCYAMFIGHATALIQSLDSSRRQYQEKYKQVEQYMSFHKLPADTRQRIHEYYEHRYQGKMFDEESILGELSEPLREEIINFTCRGLVAHMPLFAHADPSFVTAVLTKLRFEVFQPGDLVVREGSVGRKMYFIQHGLLSVLARGARDTRLTDGSYFGEICLLTRGRRTASVRADTYCRLYSLSVDHFNAVLEEFPMMRRAFETVAMDRLLRIGKKNSILQRKRSEPSPGSSGGIMEQHLVQHDRDMARGVRGRAPSTGAQLSGKPVLWEPLVHAPLQAAAVTSNVAIALTHQRGPLPLSPDSPATLLARSAWRSAGSPASPLVPVRAGPWASTSRLPAPPARTLHASLSRAGRSQVSLLGPPPGGGGRRLGPRGRPLSASQPSLPQRATGDGSPGRKGSGSERLPPSGLLAKPPRTAQPPRPPVPEPATPRGLQLSANM

SEQ ID NO: 12 - protein, 806 aa, Homo sapiens
MAKSSSLNVRVVEGRALPAKDVSGSSDPYCLVKVDDEVVARTATVWRSLGPFWGEEYTVHLPLDFHQLAFYVLDEDTVGHDDIIGKISLSREAITADPRGIDSWINLSRVDPDAEVQGEICLSVQMLEDGQGRCLRCHVLQARDLAPRDISGTSDPFARVFWGSQSLETSTIKKTRFPHWDEVLELREMPGAPSPLRVELWDWDMVGKNDFLGMVEFSPKTLQQKPPKGWFRLLPFPRAEEDSGGNLGALRVKVRLIEDRVLPSQCYQPLMELLMESVQGPAEEDTASPLALLEELTLGDCRQDLATKLVKLFLGRGLAGRFLDYLTRREVARTMDPNTLFRSNSLASKSMEQFMKLVGMPYLHEVLKPVISRVFEEKKYMELDPCKMDLGRTRRISFKGALSEEQMRETSLGLLTGYLGPIVDAIVGSVGRCPPAMRLAFKQLHRRVEERFPQAEHQQDVKYLAISGFLFLRFFAPAILTPKLFDLRDQHADPQTSRSLLLLAKAVQSIGNLGQQLGQGKELWMAPLHPFLLQCVSRVRDFLDRLVDVDGDEEAGVPARALFPPSAIVREGYLLKRKEEPAGLATRFAFKKRYVWLSGETLSFSKSPEWQMCHSIPVSHIRAVERVDEGAFQLPHVMQVVTQDGTGALHTTYLQCKNVNELNQWLSALRKASAPNPNKLAACHPGAFRSARWTCCLQAERSAAGCSRTHSAVTLGDWSDPLDPDAEAQTVYRQLLLGRDQLRLKLLEDSNMDTTLEADTGACPEVLARQRAATARLLEVLADLDRAHEEFQQQERGKAALGPLGP

SEQ ID NO: 13 - protein, 244 aa, Homo sapiens
MAAAASPAFLLCLPLLHLLSGWSRAGWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPGTTQPKAMATTLSPWSLLIIFLCFILAGR

SEQ ID NO: 14 - protein, 206 aa, Homo sapiens
MEVDINGESRSTLTTLPFPGAEANSPGKAEAEKPRCSSTPCSPMRRTVSGYQILHMDSNYLVGFTTGEELLKLAQKCTGGEESKAEAMPSLRSKQLDAGLARSSRLYKTRSRYYQPYEIPAVNGRRRRRMPSSGDKCTKSLPYEPYKALHGPLPLCLLKGKRAHSKSLDYLNLDKMIKEPADTEVLQYQLQHLTLRGDRVFARNNT

SEQ ID NO: 15 - protein, 678 aa, Homo sapiens
MAAPGPLPAAALSPGAPTPRELMHGVAGVTSRAGRDREAGSVLPAGNRGARKASRRSSSRSMSRDNKFSKKDCLSIRNVVASIQTKEGLNLKLISGDVLYIWADVIVNSVPMNLQLGGGPLSRAFLQKAGPMLQKELDDRRRETEEKVGNIFMTSGCNLDCKAVLHAVAPYWNNGAETSWQIMANIIKKCLTTVEVLSFSSITFPMIGTGSLQFPKAVFAKLILSEVFEYSSSTRPITSPLQEVHFLVYTNDDEGCQAFLDEFTNWSRINPNKARIPMAGDTQGVVGTVSKPCFTAYEMKIGAITFQVATGDIATEQVDVIVNSTARTFNRKSGVSRAILEGAGQAVESECAVLAAQPHRDFIITPGGCLKCKIIIHVPGGKDVRKTVTSVLEECEQRKYTSVSLPAIGTGNAGKNPITVADNIIDAIVDFSSQHSTPSLKTVKVVIFQPELLNIFYDSMKKRDLSASLNFQSTFSMTTCNLPEHWTDMNHQLFCMVQLEPGQSEYNTIKDKFTRTCSSYAIEKIERIQNAFLWQSYQVKKRQMDIKNDHKNNERLLFHGTDADSVPYVNQHGFNRSCAGKNAVSYGKGTYFAVDASYSAKDTYSKPDSNGRKHMYVVRVLTGVFTKGRAGLVTPPPKNPHNPTDLFDSVTNNTRSPKLFVVFFDNQAYPEYLITFTA

SEQ ID NO: 16 - protein, 816 aa, Homo sapiens
MSRPQGLLWLPLLFTPVCVMLNSNVLLWLTALAIKFTLIDSQAQYPVVNTNYGKIRGLRTPLPNEILGPVEQYLGVPYASPPTGERRFQPPEPPSSWTGIRNTTQFAAVCPQHLDERSLLHDMLPIWFTANLDTLMTYVQDQNEDCLYLNIYVPTEDDIHDQNSKKPVMVYIHGGSYMEGTGNMIDGSILASYGNVIVITINYRLGILGFLSTGDQAAKGNYGLLDQIQALRWIEENVGAFGGDPKRVTIFGSGAGASCVSLLTLSHYSEGLFQKAIIQSGTALSSWAVNYQPAKYTRILADKVGCNMLDTTDMVECLRNKNYKELIQQTITPATYHIAFGPVIDGDVIPDDPQILMEQGEFLNYDIMLGVNQGEGLKFVDGIVDNEDGVTPNDFDFSVSNFVDNLYGYPEGKDTLRETIKFMYTDWADKENPETRRKTLVALFTDHQWVAPAVATADLHAQYGSPTYFYAFYHHCQSEMKPSWADSAHGDEVPYVFGIPMIGPTELFSCNFSKNDVMLSAVVMTYWTNFAKTGDPNQPVPQDTKFIHTKPNRFEEVAWSKYNPKDQLYLHIGLKPRVRDHYRATKVAFWLELVPHLHNLNEIFQYVSTTTKVPPPDMTSFPYGTRRSPAKIWPTTKRPAITPANNPKHSKDPHKTGPEDTTVLIETKRDYSTELSVTIAVGASLLFLNILAFAALYYKKDKRRHETHRRPSPQRNTTNDIAHIQNEEIMSLQMKQLEHDHECESLQAHDTLRLTCPPDYTLTLRRSPDDIPLMTPNTITMIPNTLTGMQPLHTFNTFSGGQNSTNLPHGHSTTRV

SEQ ID NO: 17 - protein, 128 aa, Homo sapiens
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 18 - protein, 166 aa, Homo sapiens
MASGVTVNDEVIKVFNDMKVRKSSTQEEIKKRKKAVLFCLSDDKRQIIVEEAKQILVGDIGDTVEDPYTSFVKLLPLNDCRYALYDATYETKESKKEDLVFIFWAPESAPLKSKMIYASSKDAIKKKFTGIKHEWQVNGLDDIKDRSTLGEKLGGNVVVSLEGKPL

SEQ ID NO: 19 - protein, 403 aa, Homo sapiens
MFSVFEEITRIVVKEMDAGGDMIAVRSLVDADRFRCFHLVGEKRTFFGCRHYTTGLTLMDILDTDGDKWLDELDSGLQGQKAEFQILDNVDSTGELIVRLPKEITISGSFQGFHHQKIKISENRISQQYLATLENRKLKRELPFSFRSINTRENLYLVTETLETVKEETLKSDRQYKFWSQISQGHLSYKHKGQREVTIPPNRVLSYRVKQLVFPNKETMKKDGASSCLGKSLGSEDSRNMKEKLEDMESVLKDLTEEKRKDVLNSLAKCLGKEDIRQDLEQRVSEVLISGELHMEDPDKPLLSSLFNAAGVLVEARAKAILDFLDALLELSEEQQFVAEALEKGTLPLLKDQVKSVMEQNWDELASSPPDMDYDPEARILCALYVVVSILLELAEGPTSVSS

SEQ ID NO: 20 - protein, 1362 aa, Homo sapiens
MSAESGPGTRLRNLPVMGDGLETSQMSTTQAQAQPQPANAASTNPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTEETEIMIVQAKGRGRGRKETGTAKPGVSTVPNTTQASTPPQTQTPQPNPPPVQATPHPFPAVTPDLIVQTPVMTVVPPQPLQTPPPVPPQPQPPPAPAPQPVQSHPPIIAATPQPVKTKKGVKRKADTTTPTTIDPIHEPPSLPPEPKTTKLGQRRESSRPVKPPKKDVPDSQQHPAPEKSSKVSEQLKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRFAKMPDEPEEPVVAVSSPAVPPPTKVVAPPSSSDSSSDSSSDSDSSTDDSEEERAQRLAELQEQLKAVHEQLAALSQPQQNKPKKKEKDKKEKKKEKHKRKEEVEENKKSKAKEPPPKKTKKNNSSNSNVSKKEPAPMKSKPPPTYESEEEDKCKPMSYEEKRQLSLDINKLPGEKLGRVVHIIQSREPSLKNSNPDEIEIDFETLKPSTLRELERYVTSCLRKKRKPQAEKVDVIAGSSKMKGFSSSESESSSESSSSDSEDSETEMAPKSKKKGHPGREQKKHHHHHHQQMQQAPAPVPQQPPPPPQQPPPPPPPQQQQQPPPPPPPPSMPQQAAPAMKSSPPPFIATQVPVLEPQLPGSVFDPIGHFTQPILHLPQPELPPHLPQPPEHSTPPHLNQHAVVSPPALHNALPQQPSRPSNRAAALPPKPARPPAVSPALTQTPLLPQPPMAQPPQVLLEDEEPPAPPLTSMQMQLYLQQLQKVQPPTPLLPSVKVQSQPPPPLPPPPHPSVQQQLQQQPPPPPPPQPQPPPQQQHQPPPRPVHLQPMQFSTHIQQPPPPQGQQPPHPPPGQQPPPPQPAKPQQVIQHHHSPRHHKSDPYSTGHLREAPSPLMIHSPQMSQFQSLTHQSPPQQNVQPKKQELRAASVVQPQPLVVVKEEKIHSPIIRSEPFSPSLRPEPPKHPESIKAPVHLPQRPEMKPVDVGRPVIRPPEQNAPPPGAPDKDKQKQEPKTPVAPKKDLKIKNMGSWASLVQKHPTTPSSTAKSSSDSFEQFRRAAREKEEREKALKAQAEHAEKEKERLRQERMRSREDEDALEQARRAHEEARRRQEQQQQQRQEQQQQQQQQAAAVAAAATPQAQSSQPQSMLDQQRELARKREQERRRREAMAATIDMNFQSDLLSIFEENLF

SEQ ID NO: 21 - protein, 490 aa, Homo sapiens
MSEVTKNSLEKILPQLKCHFTWNLFKEDSVSRDLEDRVCNQIEFLNTEFKATMYNLLAYIKHLDGNNEAALECLRQAEELIQQEHADQAEIRSLVTWGNYAWVYYHLGRLSDAQIYVDKVKQTCKKFSNPYSIEYSELDCEEGWTQLKCGRNERAKVCFEKALEEKPNNPEFSSGLAIAMYHLDNHPEKQFSTDVLKQAIELSPDNQYVKVLLGLKLQKMNKEAEGEQFVEEALEKSPCQTDVLRSAAKFYRRKGDLDKAIELFQRVLESTPNNGYLYHQIGCCYKAKVRQMQNTGESEASGNKEMIEALKQYAMDYSNKALEKGLNPLNAYSDLAEFLETECYQTPFNKEVPDAEKQQSHQRYCNLQKYNGKSEDTAVQHGLEGLSISKKSTDKEEIKDQPQNVSENLLPQNAPNYWYLQGLIHKQNGDLLQAAKCYEKELGRLLRDAPSGIGSIFLSASELEDGSEEMGQGAVSSSPRELLSNSEQLN

SEQ ID NO: 22 - protein, 677 aa, Homo sapiens
MDDPDCDSTWEEDEEDAEDAEDEDCEDGEAAGARDADAGDEDEESEEPRAARPSSFQSRMTGSRNWRATRDMCRYRHNYPDLVERDCNGDTPNLSFYRNEIRFLPNGCFIEDILQNWTDNYDLLEDNHSYIQWLFPLREPGVNWHAKPLTLREVEVFKSSQEIQERLVRAYELMLGFYGIRLEDRGTGTVGRAQNYQKRFQNLNWRSHNNLRITRILKSLGELGLEHFQAPLVRFFLEETLVRRELPGVRQSALDYFMFAVRCRHQRRQLVHFAWEHFRPRCKFVWGPQDKLRRFKPSSLPHPLEGSRKVEEEGSPGDPDHEASTQGRTCGPEHSKGGGRVDEGPQPRSVEPQDAGPLERSQGDEAGGHGEDRPEPLSPKESKKRKLELSRREQPPTEPGPQSASEVEKIALNLEGCALSQGSLRTGTQEVGGQDPGEAVQPCRQPLGARVADKVRKRRKVDEGAGDSAAVASGGAQTLALAGSPAPSGHPKAGHSENGVEEDTEGRTGPKEGTPGSPSETPGPSPAGPAGDEPAESPSETPGPRPAGPAGDEPAESPSETPGPRPAGPAGDEPAESPSETPGPSPAGPTRDEPAESPSETPGPRPAGPAGDEPAESPSETPGPRPAGPAGDEPAESPSETPGPSPAGPTRDEPAKAGEAAELQDAEVESSAKSGKP

SEQ ID NO: 23 - protein, 293 aa, Homo sapiens
MRVLVGGGTGFIGTALTQLLNARGHEVTLVSRKPGPGRITWDELAASGLPSCDAAVNLAGENILNPLRRWNETFQKEVIGSRLETTQLLAKAITKAPQPPKAWVLVTGVAYYQPSLTAEYDEDSPGGDFDFFSNLVTKWEAAARLPGDSTRQVVVRSGVVLGRGGGAMGHMLLPFRLGLGGPIGSGHQFFPWIHIGDLAGILTHALEANHVHGVLNGVAPSSATNAEFAQTLGAALGRRAFIPLPSAVVQAVFGRQRAIMLLEGQKVIPQRTLATGYQYSFPELGAALKEIVA

SEQ ID NO: 24 - protein, 1349 aa, Homo sapiens
MSAPVGPRGRLAPIPAASQPPLQPEMPDLSHLTEEERKIILAVMDRQKKEEEKEQSVLKKLHQQFEMYKEQVKKMGEESQQQQEQKGDAPTCGICHKTKFADGCGHNCSYCQTKFCARCGGRVSLRSNKVMWVCNLCRKQQEILTKSGAWFYNSGSNTPQQPDQKVLRGLRNEEAPQEKKPKLHEQTQFQGPSGDLSVPAVEKSRSHGLTRQHSIKNGSGVKHHIASDIASDRKRSPSVSRDQNRRYDQREEREEYSQYATSDTAMPRSPSDYADRRSQHEPQFYEDSDHLSYRDSNRRSHRHSKEYIVDDEDVESRDEYERQRREEEYQSRYRSDPNLARYPVKPQPYEEQMRIHAEVSRARHERRHSDVSLANADLEDSRISMLRMDRPSRQRSISERRAAMENQRSYSMERTREAQGPSSYAQRTTNHSPPTPRRSPLPIDRPDLRRTDSLRKQHHLDPSSAVRKTKREKMETMLRNDSLSSDQSESVRPPPPKPHKSKKGGKMRQISLSSSEEELASTPEYTSCDDVEIESESVSEKGDMDYNWLDHTSWHSSEASPMSLHPVTWQPSKDGDRLIGRILLNKRLKDGSVPRDSGAMLGLKVVGGKMTESGRLCAFITKVKKGSLADTVGHLRPGDEVLEWNGRLLQGATFEEVYNIILESKPEPQVELVVSRPIGDIPRIPDSTHAQLESSSSSFESQKMDRPSISVTSPMSPGMLRDVPQFLSGQLSSQSLSRRTTPFVPRVQIKLWFDKVGHQLIVTILGAKDLPSREDGRPRNPYVKIYFLPDRSDKNKRRTKTVKKTLEPKWNQTFIYSPVHRREFRERMLEITLWDQARVREEESEFLGEILIELETALLDDEPHWYKLQTHDVSSLPLPHPSPYMPRRQLHGESPTRRLQRSKRISDSEVSDYDCDDGIGVVSDYRHDGRDLQSSTLSVPEQVMSSNHCSPSGSPHRVDVIGRTRSWSPSVPPPQSRNVEQGLRGTRTMTGHYNTISRMDRHRVMDDHYSPDRDRDCEAADRQPYHRSRSTEQRPLLERTTTRSRSTERPDTNLMRSMPSLMTGRSAPPSPALSRSHPRTGSVQTSPSSTPVAGRRGRQLPQLPPKGTLDRKAGGKKLRSTVQRSTETGLAVEMRNWMTRQASRESTDGSMNSYSSEGNLIFPGVRLASDSQFSDFLDGLGPAQLVGRQTLATPAMGDIQVGMMDKKGQLEVEIIRARGLVVKPGSKTLPAPYVKVYLLDNGVCIAKKKTKVARKTLEPLYQQLLSFEESPQGKVLQIIVWGDYGRMDHKSFMGVAQILLDELELSNMVIGWFKLFPPSSLVDPTLAPLTRRASQSSLESSTGPSYSRS

SEQ ID NO: 25 - protein, 380 aa, Homo sapiens
MRNCKMARVASVLGLVMLSVALLILSLISYVSLKKENIFTTPKYASPGAPRMYMFHAGFRSQFALKFLDPSFVPITNSLTQELQEKPSKWKFNRTAFLHQRQEILQHVDVIKNFSLTKNSVRIGQLMHYDYSSHKYVFSISNNFRSLLPDVSPIMNKHYNICAVVGNSGILTGSQCGQEIDKSDFVFRCNFAPTEAFQRDVGRKTNLTTFNPSILEKYYNNLLTIQDRNNFFLSLKKLDGAILWIPAFFFHTSATVTRTLVDFFVEHRGQLKVQLAWPGNIMQHVNRYWKNKHLSPKRLSTGILMYTLASAICEEIHLYGFWPFGFDPNTREDLPYHYYDKKGTKFTTKWQESHQLPAEFQLLYRMHGEGLTKLTLSHCA

SEQ ID NO: 26 - protein, 212 aa, Homo sapiens
MKPPSSIQTSEFDSSDEEPIEDEQTPIHISWLSLSRVNCSQFLGLCALPGCKFKDVRRNVQKDTEELKSCGIQDIFVFCTRGELSKYRVPNLLDLYQQCGIITHHHPIADGGTPDIASCCEIMEELTTCLKNYRKTLIHCYGGLGRSCLVAACLLLYLSDTISPEQAIDSLRDLRGSGAIQTIKQYNYLHEFRDKLAAHLSSRDSQSRSVSR

SEQ ID NO: 27 - protein, 378 aa, Homo sapiens
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTL

SEQ ID NO: 28 - protein, 156 aa, Homo sapiens
MAGQFRSYVWDPLLILSQIVLMQTVYYGSLGLWLALVDGLVRSSPSLDQMFDAEILGFSTPPGRLSMMSFILNALTCALGLLYFIRRGKQCLDFTVTVHFFHLLGCWFYSSRFPSALTWWLVQAVCIALMAVIGEYLCMRTELKEIPLNSAPKSNV

SEQ ID NO: 29 - protein, 165 aa, Homo sapiens
MAPNASCLCVHVRSEEWDLMTFDANPYDSVKKIKEHVRSKTKVPVQDQVLLLGSKILKPRRSLSSYGIDKEKTIHLTLKVVKPSDEELPLFLVESGDEAKRHLLQVRRSSSVAQVKAMIETKTGIIPETQIVTCNGKRLEDGKMMADYGIRKGNLLFLACYCIGG

SEQ ID NO: 30 - protein, 651 aa, Homo sapiens
MSGVRAVRISIESACEKQVHEVGLDGTETYLPPLSMSQNLARLAQRIDFSQGSGSEEEEAAGTEGDAQEWPGAGSSADQDDEEGVVKFQPSLWPWDSVRNNLRSALTEMCVLYDVLSIVRDKKFMTLDPVSQDALPPKQNPQTLQLISKKKSLAGAAQILLKGAERLTKSVTENQENKLQRDFNSELLRLRQHWKLRKVGDKILGDLSYRSAGSLFPHHGTFEVIKNTDLDLDKKIPEDYCPLDVQIPSDLEGSAYIKVSIQKQAPDIGDLGTVNLFKRPLPKSKPGSPHWQTKLEAAQNVLLCKEIFAQLSREAVQIKSQVPHIVVKNQIISQPFPSLQLSISLCHSSNDKKSQKFATEKQCPEDHLYVLEHNLHLLIREFHKQTLSSIMMPHPASAPFGHKRMRLSGPQAFDKNEINSLQSSEGLLEKIIKQAKHIFLRSRAAATIDSLASRIEDPQIQAHWSNINDVYESSVKVLITSQGYEQICKSIQLQLNIGVEQIRVVHRDGRVITLSYQEQELQDFLLSQMSQHQVHAVQQLAKVMGWQVLSFSNHVGLGPIESIGNASAITVASPSGDYAISVRNGPESGSKIMVQFPRNQCKDLPKSDVLQDNKWSHLRGPFKEVQWNKMEGRNFVYKMELLMSALSPCLL

SEQ ID NO: 31 - protein, 403 aa, Homo sapiens
MASQPPPPPKPWETRRIPGAGPGPGPGPTFQSADLGPTLMTRPGQPALTRVPPPILPRPSQQTGSSSVNTFRPAYSSFSSGYGAYGNSFYGGYSPYSYGYNGLGYNRLRVDDLPPSRFVQQAEESSRGAFQSIESIVHAFASVSMMMDATFSAVYNSFRAVLDVANHFSRLKIHFTKVFSAFALVRTIRYLYRRLQRMLGLRRGSENEDLWAESEGTVACLGAEDRAATSAKSWPIFLFFAVILGGPYLIWKLLSTHSDEVTDSINWASGEDDHVVARAEYDFAAVSEEEISFRAGDMLNLALKEQQPKVRGWLLASLDGQTTGLIPANYVKILGKRKGRKTVESSKVSKQQQSFTNPTLTKGATVADSLDEQEAAFESVFVETNKVPVAPDSIGKDGEKQDL

SEQ ID NO: 32 - protein, 530 aa, Homo sapiens
MEEDSLYLGGEWQFNHFSKLTSSRLDAAFAEIQRTSLPEKSPLSCETRVDLCDDLVPEARQLAPREKLPLSSRRPAAVGAGLQNMGNTCYVNASLQCLTYTPPLANYMLSREHSQTCHRHKGCMLCTMQAHITRALHNPGHVIQPSQALAAGFHRGKQEDAHEFLMFTVDAMKKACLPGHKQVDHPSKDTTLIHQIFGGYWRSQIKCLHCHGISDTFDPYLDIALDIQAAQSVQQALEQLVKPEELNGENAYHCGVCLQRAPASKTLTLHTSAKVLILVLKRFSDVTGNKIAKNVQYPECLDMQPYMSQQNTGPLVYVLYAVLVHAGWSCHNGHYFSYVKAQEGQWYKMDDAEVTAASITSVLSQQAYVLFYIQKSEWERHSESVSRGREPRALGAEDTDRRATQGELKRDHPCLQAPELDEHLVERATQESTLDRWKFLQEQNKTKPEFNVRKVEGTLPPDVLVIHQSKYKCGMKNHHPEQQSSLLNLSSSTPTHQESMNTGTLASLRGRARRSKGKNKHSKRALLVCQ

SEQ ID NO: 33 - protein, 442 aa, Homo sapiens
MENWSKDITHSYLEQETTGINKSTQPDEQLTMNSEKSMHRKSTELVNEITCENTEWPGQRSTNFQIISSYPDDESVYCTTEKYNVMEHRHNDMHYECMTPCQVTSDSDKEKTIAFLLKELDILRTSNKKLQQKLAKEDKEQRKLKFKLELQEKETEAKIAEKTAALVEEVYFAQKERDEAVMSRLQLAIEERDEAIARAKHMEMSLKVLENINPEENDMTLQELLNRINNADTGIAIQKNGAIIVDRIYKTKECKMRITAEEMSALIEERDAALSKCKRLEQELHHVKEQNQTSANNMRHLTAENNQERALKAKLLSMQQARETAVQQYKKLEEEIQTLRVYYSLHKSLSQEENLKDQFNYTLSTYEEALKNRENIVSITQQQNEELATQLQQALTERANMELQLQHAREASQVANEKVQKLERLVDVLRKKVGTGTMRTVI

SEQ ID NO: 34 - protein, 322 aa, Homo sapiens [sequence omitted]

SEQ ID NO: 35 - protein, 530 aa, Homo sapiens
MEDDSLYLGGEWQFNHFSKLTSSRPDAAFAEIQRTSLPEKSPLSSEARVDLCDDLAPVARQLAPRKKLPLSSRRPAAVGAGLQNMGNTCYENASLQCLTYTPPLANYMLSREHSQTCQRPKCCMLCTMQAHITWALHSPGHVIQPSQALAAGFHRGKQEDAHEFLMFTVDAMKKACLPGHKQVDHHSKDTTLIHQIFGGCWRSQIKCLHCHGISDTFDPYLDIALDIQAAQSVKQALEQLVKPEELNGENAYHCGLCLQRAPASKTLTLHTSAKVLILVLKRFSDVTGNKLAKNVQYPECLDMQPYMSQQNTGPLVYVLYAVLVHAGWSCHDGHYFSYVKAQEGQWYKMDDAKVTACSITSVLSQQAYVLFYIQKSEWERHSESVSRGREPRALGAEDTDRRATQGELKRDHPCLQAPELDERLVERATQESTLDHWKFPQEQNKTKPEFNVRKVEGTLPPNVLVIHQSKYKCGMKNHHPEQQSSLLNLSSTTRTDQESVNTGTLASLQGRTRRSKGKNKHSKRALLVCQ

SEQ ID NO: 36 - protein, 3685 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSDLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLRELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 37 - DNA, 4119 bp, Homo sapiens
  source 1..4119: mol_type = other DNA
gagtttggagcaagtaactgtcagtgaggttgcagttggtctgggctgtttggctgtgagcgaaatagctgccccccacttctcacttgcacaccacgggatactcctcctgaggctccggatgattcagatggactgtgaaaaacaacaagatggatgatcatatggagattgcttctaacataaatctgcataaaaatttttctgaaacatggctggaatatttaaggagttttttttcagtactgaggacctccctgaagtcattctaacattgtctttgatcagctccattggagcatttttgaaccggcacttggaagactttccaattcctgtccctgtgatattatttttacttggatgcagttttgaagtattaagctttacatcttcacaggtccaaagatacgcaaacgccatacaatggatgagtccagacttattttttcgtatatttacaccagtagttttctttactactgcatttgacatggatacgtacatgcttcaaaagttattttggcagatacttttaatttcaattcccggctttttggttaattatatcttagttctttggcatctggcatctgtaaatcaattacttttgaagcctacccaatggttattattttcagctatccttgtgagttcagatcccatgctaaccgcagctgctataagagaccttgggctttctagaagcctcatcagtttaattaatggagaaagtctgatgacctctgttatatcattaattacatttactagtattatggattttgaccaaagactacaaagtaaaagaaaccataccttagctgaagagatcgtgggtggaatttgttcatatattatagcaagtttcttgtttggaattctaagttcaaaactgattcaattttggatgtcaactgtttttggtgatgatgtcaatcatataagtctcatcttttcaattctgtatctcatcttttatatttgtgagttagttggaatgtcaggaatatttactctggccattgtgggacttcttttaaattctacaagttttaaagcagcaattgaagaaacacttcttcttgaattctggacttttctatcacgtattgcttttctcatggtgtttactttctttggacttctaattcctgcacatacatatttgtatatagaatttgttgatatatactattcattaaatatctacttaacattgattgttttaagatttctgacccttcttttaataagccctgttttgtctcgagttggtcatgagttcagttggcgctggatattcataatggtctgtagtgaaatgaaggggatgcctaatataaacatggcccttctgcttgcctactctgatctttattttggatctgacaaagaaaaatctcaaatattatttcatggagtgttagtatgcctaataacccttgttgtcaatagatttattttgccagtggcagttactatactaggtcttcgtgatgccacatcaacaaaatataaatcggtttgttgcacatttcaacactttcaagagctaaccaagtctgcagcctctgcccttaaatttgacaaagatcttgctaatgctgattggaacatgattgagaaagcaattacacttgaaaacccatacatgttgaacgaagaagaaacaacagaacatcagaaggtgaaatgtccacactgtaacaaggaaatagatgagatctttaacactgaagcaatggagctggccaacaggcgtctcttgtcagcacaaatagcaagctaccagagacaatacaggaatgagattctgtcccagagtgctgtccaggtgttggttggtgcagcagaaagttttggtgagaagaagggaaaatgtatgagtcttgatacaataaagaattattctgaaagccaaaaaacagttacctttgctagaaaactactacttaattgggtgtataataccagaaaggaaaaagagggcccatcaaaatacttcttttttcgtatatgccatacaatagtatttactgaggaatttgaacatgttggataccttgtgatattaatgaatatatttccctttataatctcttggatatcccagttaaatgtaatctaccacagcgaattaaaacacactaactactgttttcttacactttatattctagaggcactacttaagatagcagcaatgaggaaggactttttttcacatgcctggaacatattcgagttagcaattacattaattggcatcttacatgtaatacttattgaaatagacaccattaagtatatttttaatgagactgaagtaatagtctttataaaagttgttcaattttttcgtatactacgcattttcaagctcatagcaccaaagttgctgcaaataatagataaaagaatgagtcatcagaagaccttttggtatggaatactaaaaggctatgtccaaggcgaagcagacataatgaccataattgatcagattacaagttctaaacagattaaacagatgttattaaagcaagtgataaggaatatggaacatgctataaaagagctaggctacttagagtatgatcacccagaaattgctgtcactgtgaaaacaaaggaagaaattaatgttatgctcaatatggctacagaaattcttaaggcttttggcttaaaaggaattattagtaaaactgaaggtgctggaattaataagttaatcatggccaaaaagaaagaggtgcttgattctcaatctattatcaggcctcttactgttgaagaagttctatatcatattccgtggctagataaaaacaaagattatataaacttcattcaggaaaaagccaaagttgtaacatttgattgtggaaatgatatatttgaagaaggtgatgagcccaaaggaatctatatcattatttcaggcatggtaaagcttgaaaaatcaaagccaggtttagggattgatcaaatggtggagtcaaaggagaaagattttccgataattgacacagactatatgctcagtggagaaataataggagagataaactgcttaactaatgaacctatgaaatattctgccacctgcaaaactgtagtggagacatgttttattcccaaaactcacttgtatgatgcttttgagcaatgctctcctctcattaaacaaaaaatgtggctaaaacttggactcgctattacagccagaaaaatcagagaacacttatcttatgaggattggaactacaatatgcaactaaagctctctaatatttatgtagtagatataccaatgagtaccaaaactgatatttatgatgaaaatctaatctatgttatcctcatacatggagctgtagaagattgtctgttacgaaaaacttatagagcacctttcttaattcctataacatgccatcagatacaaagtattgaagatttcacaaaagtagtgattattcaaactccgattaacatgaaaacattcagaaggaatattagaaagtttgttcctaaacataaaagttatcttacaccaggattaataggttcagttggaacattggaagaaggcattcaagaagaaagaaatgttaaggaggatggagcacacagtgccgccactgccaggagtccccagccttgctccctgctggggacaaagttcaactgtaaggagtcccctagaataaacctaaggaaagtcaggaaagagtaagactgttaagaagaccgaagcatgtattaatgctgtggctatgagaggcctcctgctgcagaaacacacttccctacatcaagaaggagtaacttcaggttggatcctgtgtggatgatcttggtgctaagcagaaaagaaatttggaccttgaaaccagcagttcaacatatatactttttgcaaaatttccttgatttaaaatatttgttattttaaatatacaaaacattttagaaaatcttagagtaaattttagtcttaaagccagaaaataagtttatagccatctagatattttgcatattgctcttacagcaataatggtttggttcactttatgaaaaataaaatgtattaaaatatagtttaaa

SEQ ID NO: 38 - DNA, 1020 bp, Homo sapiens
  source 1..1020: mol_type = other DNA
catgaaggtggctgatggtgtgattcaagattgaactggaagttcaaggattctcaactctcagctggaactcatatcaacacctgagaaatggaaaatcaaaacaatgtgactgaattcattcttctgggtctcacagagaacctggagctgtggaaaatattttctgctgtgtttcttgtcatgtatgtagccacagtgctggaaaatctacttattgtggtaactattatcacaagtcagagtctgaggtcacctatgtatttttttcttaccttcttgtcccttttggatgtcatgttctcatctgtcgttgcccccaaggtgattgtagacaccctctccaagagcactaccatctctctcaaaggctgcctcacccagctgtttgtggagcatttctttggtggtgtggggatcatcctcctcactgtgatggcctatgaccgctacgtggccatctgtaagcccctgcactacacgatcatcatgagtccacgggtgtgctgcctaatggtaggaggggcttgggtggggggatttatgcacgcaatgatacaacttctcttcatgtatcaaatacccttctgtggtcctaatatcatagatcactttatatgtgatttgtttcagttgttgacacttgcctgcacggacacccacatcctgggcctcttagttaccctcaacagtgggatgatgtgtgtggccatctttcttatcttaattgcgtcctacacggtcatcctatgctccctgaagtcttacagctctaaagggcggcacaaagccctctctacctgcagctcccacctcacggtggttgtattgttctttgtcccctgtattttcttgtacatgaggcctgtggtcactcaccccatagacaaggcaatggctgtgtcagactcaatcatcacacccatgttaaatcccttgatctatacactgaggaatgcagaggtgaaaagtgccatgaagaaactctggatgaaatgggaggctttggctgggaaataa

SEQ ID NO: 39 - DNA, 1352 bp, Homo sapiens
  source 1..1352: mol_type = other DNA
actctgtcacccaggctggagtgaagtggtacgattacggctcactgcaatccctgcctcccaaattccagtgattctcgtgcctcagcctcctgagtagccgaaattacagacgtgtgccaccatgcttggctaattttttggatttttagtagagatggggtttcactatgttggccaggctagtcttgaactcctggcctgaagcaatccgcccacctcagcctcccaaagtgctgagattataggcacgagccactacacctggccacaaaattctttaaagaagccaatcccatcctccctcaagagccaaggggccacctcaccctcttgttacagcagatcctgcctcccacagtcaccctgctcccaagtgcaacctctgtctgaccctgcatggtgtgcggtgccctcctgcctcaggccgcgaagaaggatctaagggcttggcttgtttgaaagaaccacaccccgaaagtaacatctttggagaaagtgatacaagagcttctgcacccacctgatagaggaagtccaaagggtgtgcgcacacacaatggtgcctgaagaagagcctcaagaccgagagaaaggactctggtggttccagttgaaggtctggtccatggcagtcgtatccatcttgctcctcagtgtctgtttcactgtgagttctgtggtgcctcacaattttatgtatagcaaaactgtcaagaggctgtccaagttacgagagtatcaacagtatcatccaagcctgacctgcgtcatggaaggaaaggacatagaagattggagctgctgcccaaccccttggacttcatttcagtctagttgctactttatttctactgggatgcaatcttggactaagagtcaaaagaactgttctgtgatgggggctgatctggtggtgatcaacaccagggaagaacaggatttcatcattcagaatctgaaaagaaattcttcttattttctggggctgtcagatccagggggtcggcgacattggcaatgggttgaccagacaccatacaatgaaaatgtcacattctggcactcaggtgaacccaataaccttgatgagcgttgtgcgataataaatttccgttcttcagaagaatggggctggaatgacattcactgtcatgtacctcagaagtcaatttgcaagatgaagaagatctacatataaatgaaatattctccctggaaatgtgtttgggttggcatccaccgttgtagaaagctaaattgattttttaatttatgtgtaagttttgtacaaggaatgcccctaaaatgtttcagcaggctgtcacctattacacttatgatataatcca

SEQ ID NO: 40 - DNA, 580 bp, Homo sapiens
  source 1..580: mol_type = other DNA
agtatcgcggacggaagatggcgtccgccacccgtctcatccagcggctgcggaactgggcgtccgggcatgacctgcaggggaagctgcagctacgctaccaggagatctccaagcgaactcagcctcctcccaagctccctgtgggtcctagccacaagctctccaacaattactattgcactcgcgatggccgccgggaatctgtgcccccttccatcatcatgtcgtcgcagaaggcgctggtgtcaggcaagccagcagagagctctgctgtagctgccactgagaagaaggcggtgactccagctcctcccataaagaggtgggagctgtcctcggaccagccttacctgtgacactgcaccctcacggccacccgactactttgcctccttggatttcctccagggagaatgtgacctaatttatgacaaatacgtagagctcaggtatcacttctagttttactttaaaaaataaaaaaatagagacagagtctcaccatgtttcccaggctgatcttgaactcctggcctcaagcgatcctcctgccttgacctcccaaagtgctgggatt

SEQ ID NO: 41 - DNA, 2185 bp, Homo sapiens
  source 1..2185: mol_type = other DNA
ctgagcatctggttctggtaaggttaaggagctataaatccttttggaaacctaaatctaagatccggctaacgagctcatatctccctcattatgtctgttctcaataactccgaagtcaagcttttccttctgattgggatcccaggactggaacatgcccacatttggttctccatccccatttgcctcatgtacctgcttgccatcatgggcaactgcaccattctctttattataaagacagagccctcgcttcatgagcccatgtattatttccttgccatgttggctgtctctgacatgggcctgtccctctcctcccttcctaccatgttgagggtcttcttgttcaatgccatgggaatttcacctaatgcctgctttgctcaagaattcttcattcatggattcactgtcatggaatcctcagtacttctaattatgtctttggaccgctttcttgccattcacaatcccttaagatacagttctatcctcactagcaacagggttgctaaaatgggacttattttagccattaggagcattctcttagtgattccatttcccttcaccttaaggagattaaaatattgtcaaaagaatcttctttctcactcatactgtcttcatcaggataccatgaagctggcctgctctgacaacaagaccaatgtcatctatggcttcttcattgctctctgtactatgctggacttggcactgattgttttgtcttatgtgctgatcttgaagactatactcagcattgcatctttggcagagaggcttaaggccctaaatacctgtgtctcccacatctgtgctgtgctcaccttctatgtgcccatcatcaccctggctgccatgcatcactttgccaagcacaaaagccctcttgttgtgatccttattgcagatatgttcttgttggtgccgccccttatgaaccccattgtgtactgtgtaaagactcgacaaatctgggagaagatcttggggaagttgcttaatgtatgtgggagataagaacttgaacaattaggtaataaattatcaaccagtaggcatttactgtcatttgctatgtgcttaatgccatagaagtcactaatgaaggactggatgatggaagtgaaaagctatgtagtgcagaatttataataaagttgagaatataactgaacaggatagaaaaaaaagtcaagagatatataagatatagaggtttaattaacattttaagggaagttgaaggaaaatacttctgtgatggagcagctggatttgagtcaacccataaagaatgaatacaatttgggcagattgagattaccattcagttagtatctattaaaaatacagatgatatacaaagtctaatctcatactgtcaaggaaaggtaaaatagctatgaaggatgctgatctgattacaatagaaagtgaaatttaacagatagcaagtgatattttggataaaataagtgaaccagattttggagcacctaaaaaaagctttgaaaagtctaaattcaggaggtgttggaatgccaatttcttctctacatgataaatttcattttaagaagagcgtgcctgtaaacatggattgaatttgggagagatgaggaaggaagattaacaggagaaagcacaataatcctgctgtgaagtgatgactcagaataggcagggagagaacaagatcatttagctgctgttgttatttttctgttataaaaccaacatgtaaattatggaaaattcacaaatttaactaagtgactaaaagataattttaaaccccctatggttttgctgtttagtttttttctgtgatttagtctttccctgcgccttaaaaaaaatcagcccctctaatatgttcttaaaaattgattcctgcaggacacgacatttggtaccacaataattttcactaaaatttatattttaaactttttttctcatgtatagaggaaatacatgatggaaaaatcaaaagagtatacagttgaaaatacaatttgaaggggggcaaacaagattgatatggcaatctctctgggattctaaggtaagagtgttgtaaacagaaaagaaaagcttttcaaaggaactggggacttgaatgatgggtttgaattttgtcttgaggatttggcataggtgactgaat

SEQ ID NO: 42 - DNA, 2562 bp, Homo sapiens
  source 1..2562: mol_type = other DNA
gaggatgttgattgttggaacacacacctgtccaggtgcaggggagctggaggctctgtgagaggagggccagctcagccacagcaggaggactgacaggggcctgatggaggagtttgtggggctgcgtgaaggctcctcagggaaccctgtgactctgcaggagctgtggggcccctgtccccgcatccgccgaggcatccgaggtggcctggagtggctgaagcagaagctcttccgcctgggcgaggactggtacttcctgatgaccctcggggtgctcatggccctggtcagctgtgccatggacttggctgttgagagtgtggtccgagcgcaccagtggctgtacagggagattggggacagccacctgctccggtatctctcctggactgtgtaccctgtggccctcgtctctttctcttcaggcttctctcagagcatcacaccctcctctggaggttctggaatcccggaggtgaagaccatgttggcgggtgtggtcttggaggactacctggatatcaagaactttggggccaaagtggtgggcctctcctgcaccctggcctgtggcagcaccctcttcctcgggaaagtgggccctttcgtgcacctgtctgtgatgatggctgcctacctgggccgtgtgcgcaccacgaccatcggggagcctgagaacaagagcaagcaaaacgaaatgctggtggcagcggcggcagtgggcgtggccacagtctttgcagctcccttcagcggcgtcctgttcagcatcgaggtcatgtcttcccacttctctgtctgggattactggaggggcttctttgcggccacctgcggggccttcatgttccggctcctggcggtcttcaacagcgagcaggagaccatcacctccctctacaagaccagtttccgggtggacgttcccttcgacctgcctgagatcttcttttttgtggcgctggggggtctctgtggcatcctgggcagcgcttacctcttctgtcagcgaatcttctttggcttcatcaggaacaataggttcagctccaaactgctggccaccagcaagcctgtgtactccgctctggccaccttggttctcgcctccatcacctacccacccagcgccggccgcttcctagcttctcggctgtccatgaagcagcatctggactcgctgttcgacaaccactcctgggcgctgatgacccagaactccagcccaccctggcccgaggagctcgacccccagcacctgtggtgggaatggtaccacccgcggttcaccatctttgggacccttgccttcttcctggttatgaagttctggatgctgattctggccaccaccatccccatgcctgccgggtacttcatgcccatctttgtctatggagctgctatcgggcgcctctttggggagactctctcttttatcttccctgagggcatcgtggctggagggatcaccaatcccatcatgccaggggggtatgctctggcaggggctgcagccttctcaggggctgtgacccacaccatctccacggcgctgctggccttcgaggtgaccggccagatagtgcatgcactgcccgtgctgatggcggtgctggcagccaacgccattgcacagagctgccagccctccttctatgatggcaccgtcattgtcaagaagctgccatacctgccacggattctgggccgcaacatcggttcccaccgcgtgagggtggagcacttcatgaaccacagcatcaccacactggccaaggacatgccactggaggaggtggtcaaggttgtgacctccacagacgtggccaagtatcccctggtggagagcacagagtcccagatcctggtgggcatagtgcgaagggcccagctggtgcaggccctgaaggctgagcctccttcctgggctcctggacaccagcagtgtctccaggacatcttggctgcaggctgccccacagaaccagtgaccctgaagctgtccccagagacttccctgcatgaggcacacaacctctttgagctgttgaaccttcattccctctttgtgacgtcgcggggcagagctgtgggctgcgtgtcctgggtggagatgaagaaagcaatttccaacctgacaaatccgccagccccaaagtgagccggcccagcaagatgaaacagggcaccccagctgacctggtactgaggttgggctgagaccctgcttctcttcccccatcaccacctgcccctccctccagcccagctccattctttggcataacaggcaactttaacctagcccagaagaggatggctcatcctgggtgggacgatggctcctgccttgaaagacaaaaatcccaccttgggcagagctgagtgtgagaagatggaaaaccagtatctgccagttgctcagtgactggccatcacattaatgaatgatgagattggagtacactgtcaccaagggcaggcacagatgccttctggggttgtctggttcccagtgagaggctcctgagaaaaataaagctggttcccaga

SEQ ID NO: 43 - DNA, 806 bp, Homo sapiens
  source 1..806: mol_type = other DNA
ctcacttccctcaacccttcccacaaactgggaggaaaactgagacctcctggtcacccgccgccgggccttttagaaactcccacaagctctgccttccctccctggtcctcttcagaccccctcttagttcttcgcggctaacggctcgcgctcggggccgggtgtggagctggaacagagggctggcaaggcgcgcatgcgcaccgagggtggagccgctgagcacagaaccggaaacttagagacaaagttcggagccccgcccccgccgcgcgccgctgagttgtctggccccgccgacccacggcccacgacccaccgacccacgaatcggcccggccgtcgcgtgcaccatgtctggctcctccagcgtcgccgctatgaagaaagtggttcaacagctccggctggaggccggactcaaccgcgtaaaagtttcccaggcagctgcagacttgaaacagttctgtctgcagaatgctcaacatgaccctctgctgactggagtatcttcaagtacaaatcccttcagaccccagaaagtctgttcctttttgtagtaaaatgaatctttcaaaggtttcccaaaccactccttatgatccagtgaatattcaagagagctacatttgaagcctgtacaaaagcttatccctgtaacacatgtgccataatatacaaacttctactttcgtcagtccttaacatctacctctctgaattttcatgaatttctatttcacaagggtaattgttttatatacactggcagcagcatacaataaaacttagtatgaaacttt

SEQ ID NO: 44 - DNA, 3330 bp, Homo sapiens
  source 1..3330: mol_type = other DNA
ctggcagtgtcatggctgcccacaggtctgcaggcactcggtacgccgctaacgcggcgaggtagctcggtgcgtctcgcggtaccagtgcgaatcatcgggctatccaggtccgagatcctagtctcctgtcggctctgaggaggatggatccttctgcggatacatgggacctcttctcacctttaatatcattatggataaacaggttttacatttatttgggctttgctgttagcattagcctttggatttgtgtccagattgtcatcaagacgcagggcaagaacttacaggaaaaatctgttccaaaagcagctcaggatttgatgacaaatggttatgtctcccttcaagagaaagacatctttgtgtctggagtgaagattttttatggttctcagactggaacagcgaagggattcgcaacagttcttgctgaagcagttacatccctggatctgcctgtggccattattaatctaaaagaatatgatccagatgatcatctgatagaagaggtgactagtaaaaatgtctgtgtcttcctggttgcgacatacactgacggcctaccaactgaaagtgcagagtggttctgcaaatggttagaggaagcatccattgattttcgatttggcaaaacttacctgaagggtatgagatatgcggtatttggcctgggaaattctgcctatgctagccacttcaacaaggttggcaaaaatgttgacaagtggctctggatgcttggcgcgcatcgtgtgatgagtcgaggggagggcgactgcgacgtggttaaaagcaagcacggcagcattgaggccgacttcagagcatggaagaccaagttcatctcccagctgcaggcacttcagaaaggggagagaaagaagtcctgtggcggccactgcaagaaaggcaaatgtgaatctcaccaacatggctcagaggagagggaggaaggatctcatgagcaggatgaattgcatcatagagacaccgaggaggaagaaccctttgagagctccagtgaagaagagtttggtggtgaggaccatcagagcctaaattccattgttgatgttgaagatttgggcaaaattatggatcatgtgaagaaagaaaagagagaaaaggaacagcaggaagagaagtctggtttgttcaggaacatggggaggaatgaagatggtgaaagaagagctatgataactcctgctctccgagaagcccttactaaacaaggttatcagttgattgggagccactcgggggtgaagctttgcaggtggacaaagtccatgctccgagggagaggaggttgttacaaacacacattctatggaattgagagccatcgctgcatggaaaccaccccgagcttggcgtgtgctaataaatgtgtcttctgttggcggcaccacaccaaccccgtgggcactgagtggcggtggaagatggaccagcctgaaatgatcttgaaggaagccattgaaaaccatcagaacatgattaagcagtttaaaggagtaccgggcgtcaaagcagaacgctttgaagaaggaatgacggtaaagcactgtgcattgtccctcgtgggagaaccaataatgtacccagagatcaacaggtttttgaagctactccaccagtgtaaaatttccagcttcctggtcacaaacgcacaatttcctgcggaaatcaggaacctcgagccggttactcagctgtatgtcagtgtggatgccagtaccaaagacagcctgaagaaaatcgaccgcccactcttcaaggatttctggcagagattccttgacagtttaaaagccttggcagtcaagcaacaacgaactgtctacagactgacgctcgtgaaagcatggaacgtggacgagctccaggcctacgcgcagctcgtgtccctggggaatcctgacttcatcgaagtgaagggcgttacctactgcggagaaagttcagcaagcagtcttaccatggcccacgtgccctggcatgaggaagtggtacagtttgtccacgagttggtggatctgatccccgaatatgaaattgcatgtgaacacgaacactctaattgcctcctgatagcacacagaaagtttaaaattggtggtgaatggtggacatggatcgattataaccgcttccaggagctcatccaggaatatgaagatagtggtggatcaaaaacgttcagcgcaaaggattatatggccagaactcctcactgggcattatttggtgccagtgaaagaggctttgatcccaaggacacaagacatcagagaaagaacaaatcaaaggctatttctggatgttgagattatctgatttcaaggtactgaaggacaaaaacttggatggcctcaaaaggttcttgaacaccactgtgattctccaaggacgaattacgtaaattatactttcatacaaaggagacgataaggcagtaaacatggagacacgggggacagcgtccacactcagagggcctgggccacagccccgatgtttcttttcagaactcagcccctttcctgattttacttctaagaggaaaattattttggggaggaactacacagtcgtgattagaatttatctgatggttttgtattataacttgtaagacctgccagaatgctagtcccgagagtgtcagacaaggaagaagtccctgggcctcttccccttacccggcccttagatttcatggagcagccacttagcattgaattgcactaccctgagctaaacgtgtctgtgctttctaagataagagcttgatccctttcttctatcttaagacagcacctcctgaaaagaatcgaagttgtcacaactctcaattattttttaaatactgcatagattgagttttggtttattaccaacccttcccagaattgcgttggatctaaaactactagatctcatcccattcccatgtaaattaccacagaccgcagtaccggggctggagcggagtgaagctgtctgctgtaagaggagtggccatgtgagggcatggagtcattagtctcacaaacacactttggactgaagaggatcatttctttttgttcgtgaggtcactgtccaggcctctcatatcatgaccagacggcgggtctccatcttctttcactcctgtggccctggctgctttacacaatctgttctataaggttcaggtgttttcaagttggaaagatcataaatactcaaaattgttttcaagttagcaagttcttttaacagtcttttatgcaaaaattgaattaataaaataatcttttgtaaaga

SEQ ID NO: 45 - DNA, 2236 bp, Homo sapiens
  source 1..2236: mol_type = other DNA
ctagtttagtccctttatcctgtgaagtaggggtcatcattagcccccttttacagaggagagaattgaggcttcgagagagagaaacttggccaggagtttccactcggtccgacgccctcggtgccccgccgggtacggtggctgggcgcggggagcggggggcggcgccggcggggccccgggcaggggcggggtcggggcgcggacaaaaccgccgcggggcggcggggtggcggacgcggccatggaggccgagggctgccgctaccaatttcgggtcgcgctgctgggggacgcggcggtgggcaagacgtcgctgctgcggagctacgtggcaggcgcgcctggcgccccggagccggagcccgagcccgagcccacggtgggcgccgagtgctaccgccgcgcgctgcagctgcgggccgggccgcgggtcaagctgcaactctgggacaccgcgggccacgagcgcttcaggtgcatcaccaggtccttttaccggaatgtggtgggtgtcctgctggtctttgatgtgacaaacaggaagtcctttgaacacatccaagactggcaccaggaggtcatggccactcagggcccggacaaggtcatcttcctgctggttggccacaagagtgacctgcagagcacccgctgtgtctcagcccaggaggccgaggagctagctgcctccctgggcatggccttcgtggagacctcggttaaaaacaactgcaatgtggacctggcctttgacaccctcgctgatgctatccagcaggccctgcagcagggggacatcaagctagaagagggctgggggggtgtccggctcatccacaagacccaaatccccaggtcccccagcaggaagcagcactcaggcccatgccagtgttgactctaggagagaaagggttaaagcagtcccagccttagcccacctggtgggatggggagtgttaatatctctctggaggacaaatgacagaagggttcatataaacagtatcctgacacagtcatgcttcctggattttggagtcgaggctttctacagaaaagaaagttctgatggccaggcatggtggctcacgcctgtaatcctagcatttttggaggccaaggacagtggatcacctgaggtcaggggttcgagaccagcctggccaacatggtgaaaccctgtctctactaaaaatacaaaaattagccaggcgtggtggtgcatgcctgtaatcccagttactccagaggctaaggcaggagaattgcttgaacctgggaggcagagattgcagtgagccaagactgcgccactgcactccagcctgggcaacagagtgagactctgtttcaaaaaaaaaaaagaaaagaaaagaaaggcctgagagaccagatgtgcaacttcctgtccttgagcctcagtgtccctatctatcgatggggctcataaaagatcccaccttgaagggaggtggtgaccacaaatgagacagtggacaggatgtgctcacccagagcctgccgcgctgtgaattgaatgacaaaagctctcattcccactccctttttcttggctgcgatgtggccactctggcagcattcctgggctcagacactgagaagccagcgtcaggaagctgatgcatgggcaaaggcaggtgcggggaattccagggggagcttggcttggaggcttcttatgtcctcaggctaaaatgattctgggcatgggattaatatgtgacgtcaaacccagggttgctggccaatgcccccccgaccaggcccaggggctgaaaaatggatgttggaggctgggatgaacatgaatgtgtagcaactatgttgggcacacagtggccactgtgatgagccaccaagatccccctttctggctggggaacccatcaaccctctccccagctgctggagtgccactggatgatggacttcagcttgccccactctctgggaaaggccctcccttcagggcagcttgtatccaaagttcatctcctggggggccttaaaggactccctcttgccccagctctggacaactctgaaagtcaaaaccaactttatcagtctctgtgggcttcattgaggacactgttgtgacatcatagccaagttatccccttgcccaatcctgcttccttttcttccccaaacaggtatccatttcaagaatatcccctaataaacatctgcacactcatctcca

SEQ ID NO: 46 - DNA, 3838 bp, Homo sapiens
  source 1..3838: mol_type = other DNA
gattccgagcctacgacgcctccgctagagcccgcggggctgcgccgactcctgctctggaggggttgcgggtacctgatggccacagagggctctaggaggccgagcgtgtaagcggggtgggcgccatggaggcagagcagcggccggcggcgggggccagcgaaggggcgacccctggactggaggcggtgcctcccgttgctcccccgcctgcgaccgcggcctcaggtccgatccccaaatctgggcctgagcctaagaggaggcaccttgggacgctgctccagcctacggtcaacaagttctcccttcgggtgttcggcagccacaaagcagtggaaatcgagcaggagcgggtgaagtcagcgggggcctggatcatccacccctacagcgacttccggttttactgggacctgatcatgctgctgctgatggtggggaacctcatcgtcctgcctgtgggcatcaccttcttcaaggaggagaactccccgccttggatcgtcttcaacgtattgtctgatactttcttcctactggatctggtgctcaacttccgaacgggcatcgtggtggaggagggtgctgagatcctgctggcaccgcgggccatccgcacgcgctacctgcgcacctggttcctggttgacctcatctcttctatccctgtggattacatcttcctagtggtggagctggagccacggttggacgctgaggtctacaaaacggcacgggccctacgcatcgttcgcttcaccaagatcctaagcctgctgaggctgctccgcctctcccgcctcatccgctacatacaccagtgggaggagatctttcacatgacctatgacctggccagtgctgtggttcgcatcttcaacctcattgggatgatgctgctgctatgtcactgggatggctgtctgcagttcctggtgcccatgctgcaggacttccctcccgactgctgggtctccatcaaccacatggtgaaccactcgtggggccgccagtattcccatgccctgttcaaggccatgagccacatgctgtgcattggctatgggcagcaggcacctgtaggcatgcccgacgtctggctcaccatgctcagcatgatcgtaggtgccacatgctacgccatgttcatcggccatgccacggcactcatccagtccctggactcttcccggcgtcagtaccaggagaagtacaagcaggtggagcagtacatgtccttccacaagctgccagcagacacgcggcagcgcatccacgagtactatgagcaccgctaccagggcaagatgttcgatgaggaaagcatcctgggcgagctgagcgagccgcttcgcgaggagatcattaacttcacctgtcggggcctggtggcccacatgccgctgtttgcccatgccgaccccagcttcgtcactgcagttctcaccaagctgcgctttgaggtcttccagccgggggatctcgtggtgcgtgagggctccgtggggaggaagatgtacttcatccagcatgggctgctcagtgtgctggcccgcggcgcccgggacacacgcctcaccgatggatcctactttggggagatctgcctgctaactaggggccggcgcacagccagtgttcgggctgacacctactgccgcctttactcactcagcgtggaccatttcaatgctgtgcttgaggagttccccatgatgcgccgggcctttgagactgtggccatggatcggctgctccgcatcggcaagaagaattccatactgcagcggaagcgctccgagccaagtccaggcagcagtggtggcatcatggagcagcacttggtgcaacatgacagagacatggctcggggtgttcggggtcgggccccgagcacaggagctcagcttagtggaaagccagtactgtgggagccactggtacatgcgccccttcaggcagctgctgtgacctccaatgtggccattgccctgactcatcagcggggccctctgcccctctcccctgactctccagccaccctccttgctcgctctgcttggcgctcagcaggctctccagcttccccgctggtgcccgtccgagctggcccatgggcatccacctcccgcctgcccgccccacctgcccgaaccctgcacgccagcctatcccgggcagggcgctcccaggtctccctgctgggtccccctccaggaggaggtggacggcggctaggacctcggggccgcccactctcagcctcccaaccctctctgcctcagcgggcaacaggcgatggctctcctgggcgtaagggatcaggaagtgagcggctgcctccctcagggctcctggccaaacctccaaggacagcccagccccccaggccaccagtgcctgagccagccacaccccggggtctccagctttctgccaacatgtaaaacctttgagtacatccagccttagttcttggggtgcagtagtatgtacccaagggcagatgcctcttggggaaggccatggggacctgaaacattgccccatggaaatgtcgaccctgtgcggacattccgcatactgccatgaagacggtctctgtgtcctcagctcaagaatcctgtagcttgtcccatcataatccattcacccgttcatcatgtgtactgagcagctaccatgttcaaggtaatatgccaggcgctgtatgtctccactgccaagtagaagtgactcaaaaccctctgacaaggatattcccttggctatggtcctgccaggtgcaggcccaggcccatgaccccacctttactaagcacaagtacttgccactgccatcactgccaagtaactagatgtctctgtttccctgccaatgatcctgcaggttctgcccggtctggttatcttcctgttcctgtagcatagccaggcactgccagtcacctgtgcccccattgctgtcagcagatgtcttgggtcctgagtgtgggtatccacttttacccgctcactgccacctgtggacactctgtgtctaccctctgagtgggaacatacttctaagttccctgcagtctctgtcctgtggtagaccatctttttgtaaactgcgagcttcctcttccctgtaccctctgccccagtcgtgaccccctaaaagttaaggggtagttggcacctccttattaatatgccagcctagatcccccccggtggaggggcaaatggctgaatccttgtgtgatatttttttcttcgcttgtttatttattcatttatttaattgtatttattcatttactaactttatgtgttaccaattaattttgtttacccattcctttatccatccctcccctccttttcaggtaaggagacaggaggagtaggaggaggcagggcctctccatgccagcctctgtggtccttgcccaaacccatcagcgcaatacttgaaccttctcccaggtaggggcaggaggagccacatgagagagggagaaggaccgcgtttacctttagagttttgttttgttttttccttctgagtttgctgttggtgcaggaataagggaaaggcccaaggtatccaagcctggggaagggcaggccagccagcacctctgccttctcagggacaagagtagtcctttaccaccctcactctgcctgtcccctctcctactctacagcattaaagactgtgggaccaggaccctaagtctcctttccttctgggtggggagttctggggttcttggtgtgtgggagaagttttataattgcttccaaacagctgggtttaaatataaaatagacacactca

SEQ ID NO: 47 - DNA, 3373 bp, Homo sapiens
  source 1..3373: mol_type = other DNA
gtgtttaactggaaactagaacgagatggaaggggatgttcaaggcccctcccttgactctgaacggacccccagggaacatgcgaccctctctctggcgacgcctcccacccaccactaatacttgctcctggaccggggggcgcggaggttggagagaggagctacccgggtctcggacaggcggcactgggaccacgaggcagggagccaggcttgaagcaggtgacatgtagacgtcccctggtccagcctcggaacctgagcgcccttctgcctggaaagtttgtggctaggcgccatggccaagagcagctccctgaatgttcgcgtggtggagggccgcgcgctgcctgccaaggacgtgtctgggagcagcgacccctactgcctagtgaaagtggacgacgaggtggtggccaggacagctactgtctggaggagcctgggccccttctggggggaggagtacacggtgcacctgcctctggatttccaccagctggccttctacgtgctggatgaggacactgtcgggcacgacgacatcatcggcaagatctcgctgagcagggaggcgattacagccgacccccgagggattgacagctggattaacttgagccgagtggacccagatgcagaagtgcagggtgagatctgcctgtcagtgcagatgctggaggatgggcagggccgctgccttcgctgccatgtgcttcaggccagggacctggctcccagagacatctctggcacatctgacccatttgcacgtgtgttttggggcagccagagcttggagacctcaaccatcaagaagactcgcttcccgcactgggatgaagtgctggagctgcgggagatgccaggtgccccgtccccactgcgggtggagctctgggactgggacatggtgggcaagaatgacttcttgggcatggtggagttctctccaaagaccctccagcagaagccacctaaaggctggttccgcctcctgccctttcccagagccgaggaggattctggggggaacctgggtgccctgcgagtgaaggtacgcctgattgaggaccgcgtcctgccctcccagtgctaccagcctctcatggagctgctcatggagtctgtgcaggggccagcagaggaggacactgctagccccttggctttgctggaagagctgaccttgggggactgccgccaggaccttgccaccaagctggtgaaactctttcttggccggggactggctgggcgctttctggactatctcacccggcgtgaggtggctcggaccatggaccccaacaccctcttccgttctaactccctggcatccaagtcgatggaacagtttatgaagctcgtgggcatgccctacctgcacgaggtcctgaagcctgtgattagccgtgtctttgaggagaagaagtacatggagctggatccctgcaagatggacctgggccgcacccggaggatctccttcaaaggcgcactctcggaggagcagatgcgggagaccagcctggggctgctgacgggctacctggggcccatcgtggacgccatcgtgggctccgtggggcgctgcccgcccgccatgcgcctcgccttcaagcagctgcaccggcgagtggaggagcgcttcccccaggccgagcaccagcaggatgtgaagtacctggccatcagtggatttctcttcttgcgattcttcgcacctgccatccttaccccaaagctgtttgaccttcgggaccaacacgcggacccccagactagccgctcactgctgttgcttgccaaggctgtgcagagcattggaaacctgggccagcagctgggccaaggcaaggaactgtggatggcccccctgcaccccttcctgctgcagtgtgtctcacgtgtgagagacttcctggaccggctggtggatgtggatggggatgaagaagctggtgtcccagccagggccctgttcccgccctcggccattgttcgagaaggctatctgctgaagcgcaaggaggagcctgccggcctggccacgcgctttgccttcaagaagcgctacgtctggctcagcggggagaccctctccttctccaagagtcctgagtggcagatgtgtcactccatccccgtgtctcacatccgcgccgtggagcgcgtagacgagggcgccttccaactgccccacgtgatgcaggtggtgacgcaggacggcacgggggcgctgcacaccacctacctccagtgcaagaatgtgaatgagctcaaccagtggctctcggccttgcgcaaggccagcgcccccaacccgaacaagctggccgcctgccaccccggtgccttccgcagcgcgcgctggacctgctgcctccaggctgagcgctcagccgccggctgcagccgtacacactcagctgtcaccctgggggactggagtgacccactggatcctgatgctgaggcccagacagtgtatcggcagctgctcctggggcgggaccagctcaggctgaaattactggaggattctaacatggatacaactctggaggcagacacaggggcctgtcctgaggtcctggcccggcaaagagcagcaactgcccgcctgctggaggtgctcgcagacctggatcgtgcccacgaggagttccagcagcaggagcgagggaaggcggccctgggcccccttggcccctaaggaaatgccagagctagcccggaaggaggagcaagagccagggggccctcttcagcgcatcctgccccgggagtctcctgtctccttggacctctttgattctgtggtttggaggctcccagagacgtgcctagtcctgtgtgccttgagtccagaactcagggcatggaagccctttggcaggggccagccttgcactgagtgaaacttgccctctggcttgattcagactggagtggataggataaggaacctgacttatttgactgagactggggtctctacttcaccaaactggcctctatccataccaaggaggccagcctggccctgagctgctggatacagctggacctgaattcctgatgcccatgtgatgttgttgccccagatgggcactaaatggcctcactccttcctgttttcatgtctgctaatccctataacctcactgattcttctgtaccctgcccttggcctaggactccaaccacaagcttccagaatcaggtgccctcaggaagaaccaaggctgggtgggggtccagtgtgccaaactcagacccttggagcctgggagaccttgggccaggctgtttatctctctctgggtctcagattaccctgtataaaaagaggagggaaagtcta

SEQ ID NO: 48 - DNA, 3211 bp, Homo sapiens
  source 1..3211: mol_type = other DNA
gtatccctgcgcgcggcgggccgggctgggcagctttataaacagccgtggtgtgagcctcgaagggaaccatcagcgcctcctgtccacggagctccaggtctacaatggcagcggccgccagccccgcgttccttctgtgcctcccgcttctgcacctgctgtctggctggtcccgggcaggatgggtcgacacacactgtctttgctatgacttcatcatcactcctaagtccagacctgaaccacagtggtgtgaagttcaaggcctggtggatgaaaggccttttcttcactatgactgtgttaaccacaaggccaaagcctttgcttctctggggaagaaagtcaatgtcacaaaaacctgggaagaacaaactgaaacactaagagacgtggtggatttccttaaagggcaactgcttgacattcaagtggagaatttaatacccattgagcccctcaccctgcaggccaggatgtcttgtgagcatgaagcccatggacacggcagaggatcttggcagttcctcttcaatggacagaagttcctcctctttgactcaaacaacagaaagtggacagcacttcatcctggagccaagaagatgacagagaagtgggagaagaacagggatgtgaccatgttcttccagaagatttcactgggggattgtaagatgtggcttgaagaatttttgatgtactgggaacaaatgctggatccaacaaaaccaccctctctggccccaggcacaacccaacccaaggccatggccaccaccctcagtccctggagccttctcatcatcttcctctgcttcattctagctggcagatgaggagagttgtttagagtgacaggtggaaagtgatatcaagaagcctctgttagcctggtctgggtcctgctctcccttcagggaggccgcctgtctactcaccactgtgcctttctggaaagcaggagttcaagccttagcaagcccagaggcccccagcagacgatgaggacattgtcggctcaacatctcaggccactcattaccttcgctcatgatcccagcagccatttttcttaacaccttctgccactttctgtcggtgctaatggatggaactcctgcacaagttttaactgaacaagaaatcccagcaaaaggcatttttttttctacttctttgattgtagaaaagcagacacttctctgaaacatgaccttattcttccaaacagtatcgctagtaaaatagcatgctggacttcagacctcagggatccttttgatgcactgaccaggaattgtgataatccttttcatttttatggctttttacagtttctcattctgtcaaccatattgaagtgaagtggcatagtcttcactcactgtaatctccagctcctgggctcaagtgatcctctagactcagcctctagaatagctgggactacaggcacatgccaccaggactggcaaactgttttatttttaggtagaggtagggtcttcctatgttgcccaggatggtcttcaaatcttggtctcgagcaatgctaccaccttggcctcccaatgctctgggattacagacatgaaccacagtgcctgttgtagaaatttttaattatttaatatgaaaatattacattcatgattattttatttagtaaataaaataatagagagcccagaaatcaacctgcacacctaccgccatctaatcttcaatagaaatgggcaatgtgggaaagactccctattcgaaaattagtgctggaatatctggccaaccatatgcagaagaatgaaactgaacccctacttctccccatatatgtaaaataattcaatatggatgaaagatttaaatataagtactaaaactgtaaaaatcctggaatataacctaagaaataccaatgtggacatagggcctggcaaagatttcatgaagaagacactaaaaacaattgcaacaaaaacagaaattgacaaatggggcctcattaaactaaagagcttctgcacagaaaaagaaactagcaacacagtaaacagacagcctgtagaatggggaaaactatttgcaaactctgcatctgatgaaggtccaatatccagaatctacaaggaacttaaacaattcaacaagcaagaagaaaaaacccaattaaaattgggcaaaggcatgaacagacacttttcaaaagaagacctacaattggccaacaaacatgaaaaaatgctcagtatcactaatcatcagataagtgcaaatcaaaactgcaatgagttaccatctcttaccagtcacaaagtcagagatggtggtgaggctgcagagcaaaagaaacagacactgttggtgggaaagcaaacttgttcagccactatggaaagcagtttggagatttctccaagaacttaaaatagaactaccattcaatcccgcaatcccactactcgggatatacccacaggaaaagaattcattttatcaaaaagacacctgcaccaatatgttcattacagtgctattctcaccagcaaggacagagaatcaatctaagtgcccaacaacagtaaattcaatgaaaaaaaatgtggtacatagatacgatggaaaactatgcagccatgaaacacaagaaaatcatgtccttttcagcaacatggatgcaactagaggctattatcctaagcaacctaatgcaagaacagaaaaccacatactgcatcttcccattggaaagtggcagctaaacattaaattcgcatgaaccacagatgctggagatcaccagaccggggagagaagaggggcacctgggctgaaaaacacacctgttgggtatcatgcttactgtctgggcgatgggatcattgggacaccaagcctcagcttctcaaattctacccatgtaacaaacctgtatatgtaccttgtattatataggttgaaattaaagatgaataaataaaataaaatgacacaaggccaaaaacaaatgggtttaactgaccagagcgagagaactctgcactatgaacccaaacccagctcaaaaagataaaatctagtcatttaagataatcataagttgtatgatgataattgtataaaaatttgtatgatgataattgtataataattatacatgaaagtgccaaaaccctacaattaaacactgtataatggaattaca

SEQ ID NO: 49 - DNA, 3222 bp, Homo sapiens
  source 1..3222: mol_type = other DNA
cccttggccgctgttcccgcaccgcggggcagcgggcctggaggcccctttgagaagtagctttccccggccggcggcacctttggctgcgtgcccggccgcgctcagggtgcgactgcccgggtcagatagcacctcagggcgagccccggcggtctgatctcgccgaccctcctcgtcctggttgcaacccgcgtgcgaggccgcccgcgccctccgagtgtctgccggtgcagtgggggtggactggccggctgtggccgtgtgtgcgcgtgtggattggggcccggtccgagccagaagcttaagcggcagatgtcgggcattgccaccctcgccccacctgtcgcgggtggactttggggcagtacctggagtagaacagaaaaattattatgtctgtgttcccttgggactcattggaaattgtacagtgacatcttctgggatttagtctggattgtgcagactggtgcttaaaatggaagtcgatattaatggagagtctagaagtaccctgaccaccttgcccttccctggggctgaggccaactccccgggaaaggcggaggcagagaagccccgctgctccagcacaccctgctccccgatgcggaggaccgtgtcaggctaccagatcctacacatggactctaactatttggttggcttcacgactggcgaggaactcctgaagttagctcagaagtgcacaggaggtgaagagagcaaagcagaagccatgccatccttacgctccaaacagctagatgcaggacttgcccgttcctctcgtttgtataaaaccagaagtaggtactaccagccatacgagattccagctgtcaatggcaggaggcgaaggcggatgccaagctcaggagacaagtgcactaaatctttaccttatgaaccttacaaggccctccatgggcctctgcctctttgtcttcttaaaggtaagagggctcactccaaatctctggactacctcaatctagataaaatgatcaaggagccagctgatacagaagtgctacagtaccagcttcaacacctaaccctccgaggggaccgtgtgtttgctaggaataatacatgaatgacttggagagagcttaaaccaatttaggtcagcctacgcttggctagaaaaaacccactgctgtactctgtacatgactcttcacactatagatggttatatcagctaagtgttcctggaacataaaaattgtttgggtcaaatttgaatacaggaatgaaatcacaggtacttgggggggggatatcattctagagcacgcaactgcaaagaaaacagaatgttgactgttagtttgtatagctttttagctaggaaaaaaaggctttggtacagtaatttcatctttatgattctgacactcaaattgggaatgttacctgcttggttgttgctgacttgatatgattcattagaaatttatatcttcagtactcaagtacttcttgaatctctgtattttactataaaatgtatgtaatgatttgttttatgaaatttagaacttgaacattgctgaattggaccactttttatttttaaatattgagtttaaatattttataactggttttgcactgaaaaaattaacatttcagattgacaagagagtaatctttcttcacttgcctcaataatgttattgagcaatgaattttttatttccgcatggaaagttattgatctctatggctgtaaaatatttctttatagcgttattaaagtgtgtcttaataaaattaaatttgggatacaaagtatttattttacaatgggtgggcgggggaaacttttccagaaagtttccaatatgacgttttcataagttgaaaaaactctccttagtgcttattttctaacttaaaattcacctggaactttaaatgggaaaggattcttttaattgtggattataggcataatactgtttgcatctgaattttctgtaagtgaataatagtttaatagaggaactcatgatttgtactattgaatgattaaactaagtatgaagtgataccattcagcatggcatcaggtcattgcagttttagttctgtgtaacacaagcactcactgaaattccagtttctaggattagtgtaggagcctaacgtgcttctactgttttaatgggttaatcctggattacttaacaatttatgtcaattgcactggtttaatttgttgctaaagaaataatgccctgggtttagtaacaaatacagctcaactattcttgaatatattttgaaaaaaaaatgtatgtaacttaccttttgtaaacgttccatttcttttttccctcatttttgactcttaaaggtgcaatttattactgaattgggatttctggcagcacagaactgctttttattttggggtctgtgagtttcttaggtattagcaatcttgcttataaaataagaacaccttttaattaatgagtgggtcattcctggtgcaattgtgatttttctttagccagaatgaatggcaaactctatttagagcaaagtaagtattagaaaaccctaggaactcttaatcaacgtttattacactttcattaaggcaaactacgtgaaagagccttggggaagttggcccatatcttactaagttgatcagatttctcgttgggctggaaatgtttcgctgttgtatattttaaagtaaattgcacctttgtaacatattgtattgacgaatgatcactaagattagctatatctatacagtcattagtttgacaagaaatagaatcctgtcagatgccaaagagtgggatttttatgtttaatgattaaacaccattatttattgacaatttaccctgtggaactgtattatttctaactatgaaataaaggggtgatgtaaacacacattgttgtgtggtgctttaaactaggtccactatcaacaggctacttactgttcaagaattccactgaagcacttattttaaggccctatttttcttaaacaaaacagtgacaacaacaatcaaaccatttacttttgatgctcattggcattttatgataaaagatgtattcatggcaatgatatgtattcaccctattaggaaacacaactggttacctatgagacctgttctgtccgtgtgcctacgttccttaataatagctaaataaaaatttgtagctttt

SEQ ID NO: 50 - DNA, 5002 bp, Homo sapiens
  source 1..5002: mol_type = other DNA
agtcccagcgagctgaggatggctgcgccaggcccccttcctgccgctgctctgagtccaggggctccgacccccagagaacttatgcacggagttgcaggtgttacttccagagccggacgagatcgggaggcggggagcgtgctgccggccgggaaccgtggggcgcggaaggcctcccggcgctcttcctcccggagtatgtccagagacaacaagttcagcaagaaagattgtctttcaatcaggaatgttgtagcttcaatccaaaccaaagaaggtctgaatctcaagttgataagtggagatgttctgtacatctgggccgatgtcattgtcaacagcgttcccatgaatcttcagcttggaggaggaccactatctcgggcatttttgcagaaagctggtcccatgctccagaaagagttagatgacagaaggcgggaaacagaggaaaaagtaggtaacatattcatgacaagcggctgcaatctggactgcaaagctgtgctccatgctgtggctccatactggaataatggagcagagacttcttggcagatcatggcaaatataatcaagaaatgtttgacaactgtagaagtgctatctttctcatcaatcacatttcccatgattggaacaggaagtttgcagtttcccaaagctgtttttgctaaactaatcctttcagaagtgttcgaatacagtagcagcacaaggccgataactagccctttacaagaagtccactttctggtatatacaaatgacgatgaaggctgtcaggcatttttagatgaattcactaactggtcaagaataaatcccaacaaggccaggattcccatggcaggagatacccaaggtgtggtcgggactgtctctaagccttgtttcacagcatatgaaatgaaaatcggtgcaattacttttcaggttgctactggagatatagccactgaacaggtagatgttattgtaaactcaacagcaaggacatttaatcggaaatcaggtgtgtcaagagctattttagaaggtgctggacaagctgtggaaagtgaatgtgctgtactagctgcacagcctcacagagattttataattacaccaggtggatgcttaaagtgcaaaataataattcatgttcctgggggaaaagatgtcaggaaaacggtcaccagtgttctagaagagtgtgaacagaggaagtacacatcggtttcccttccagccattggaacaggaaatgccggaaaaaaccctatcacagttgctgataacataatcgatgctattgtagacttctcatcacaacattccaccccatcattaaaaacagttaaagttgtcatttttcaacctgagctgctaaatatattctacgacagcatgaaaaaaagagacctctctgcatcactgaactttcagtccacattctccatgactacatgtaatcttcctgaacactggactgacatgaatcatcagctgttttgcatggtccagctagagccaggacaatcagaatataataccataaaggacaagttcacccgaacttgttcttcctacgcaatagagaagattgagaggatacagaatgcatttctctggcagagctaccaggtaaagaaaaggcaaatggatatcaagaatgaccataagaataatgagagactcctcttccatgggacagatgcagactcagtgccatatgtcaatcagcacggctttaatagaagttgtgctgggaaaaatgctgtatcctatggaaaaggaacctattttgctgtggatgccagttattctgccaaggacacctactccaagccagacagcaatgggagaaagcacatgtacgttgtgcgagtacttactggagtcttcacaaagggacgtgcaggattagtcacccctccacccaagaatcctcacaatcccacagatctctttgactcagtgacaaacaatacacgatctccaaagctatttgtggtattctttgataatcaggcttacccagaatatctcataactttcacggcttaaaaatatttttatcatcaaagagatgatttaagtcatctgtaagaacaacatgcaatctttgtctttgcttctggcctgtgtaagcagatgaaagtttcccttttaggtgccaaaatgctgaaaattacctttttaaagtgctctattgctgcgatttgtagcatacctttttttctcagcaaattgatgggtggaagctgagaaatgtatggtaaatgtcacagagctacaaccattcacagacaccaaatctctaggagaataaaaagcacattattctttttctatcagaaaaaaacaagatgcatcaccttaaaaccaagatgacattgttcttcttggaacatgttaagacatcgaatggtggcgggttaaactgtactgcttaagtggagcggctaccgttatgcatctatcacagttggggattttgccttattaaggaaaacttgtcaatagttcagctgaaatgactgaatcacagaatattaactctgttatggaacaaatcataacagattttacctgtttacatttcaggtaaaaatgtatcgcattgttatctaatattaaaaaattacccccaattttagtgacttaatcccacacagtctttatgggtcaggaattcaggcatggcttacctggatcattctgctagggtctctctgaagttacagacaagatgtcaggggatgtggtcgtttgaaggcttgtctgggctggaggtctatttccaaggtgaatcactcacatacctggcacgtttctgtcaggtattggcagtcctcagttcctctcctctcaggcctctccacaggctgcttgagtgtcctcatgacacaacagttggcttactccagagtgagcaactcaagagagagcaaggcagaagctaccaaatctttatgtttgaagtcatgcaccatcttttccacgagtatcctgttgattattttgatcagcttgttcagtctgggagggaattgcacaagggcatgaatactccactggcaaggatcattgggggccatcttggaagctgtgtgaatgagcaaatgaatgcacagatagaatattagcagtgacaatgatgctagaggtcacctaccccactgtcctcttgtccttctcccccaaccctcccctgctcccaggcaagaagccctctagcctctgcttgatcactttcagcactcaacatcttcagggaacctattccgccgtgggacagtgttaattagtggaaaactctttttcaaaagttgaaatcagttcctctgtgtctattacctgctgatcactgtccagacttctggaggacacagagcaagttttattcctcttactgatggtagcctttcagatccatcccttccctccagtatattagagttacgtaaattcttaaaatgcttagcagctcatttatcctgaagcatcacttttgaagagttacagacatttaagaagtatttacattatcataaataaattacatgcgcatttaaagagaacagaaaagtataaagaaaaataactcaccaaactcactcataattccagcactaagggaaaaccactgccaatttctcatgtctcccttccagtttcttctctgtccaaattcaacaaagtcaaaatcacattttgttctgtcacaaatcatataatattatttctaccatttttctttttttaatctttttttattatactttaagttttagggtacatgtgcacaatgtgcagtttagttacatatgtaaacatgtgccatgttggtgtgctgcacccagtaactcatcatttagcattaggtatatctccaaatgctatccctctcccctctccccaccccacaacaggccccggtgtgtgatgttccccttcctgtgtccatgtgttctcattgttcaattcccacctgtgagtgagaacatgtggtgtttggttttttgtccttgcgatagtttgctgagaatgatggtttccagcttcatccatgtccctacaaaggacatgaactcatcattttttatggctgcatagtattccatggtgtatatgtgccacattttcttaatccagtctatcattgttggacatttgggttggttccaagtctttgctattgtgaatagtgccgcaataaacatacgtgtgcatgtgtctttacagcagcatgatttataatcctttgggtatatacccagtaatgggattgctgggtcaaatggtatttctagttctagatccccgaggaatcgccacactgacttccacaatggttgaactagtttacagtcccaccaacagtgtaaaagtgttcctatttctccacatcctctccagcacctgttgcttcctaactttttaatgattgccattctaacgggtgtgggatggtatctcattgttgttttgatttgcatttctctgatggccagtgatgatgagcattttttcatgtgttttttggctgcataaatgtcttcttttgagaagtgtccgttcatatcctttgcccactttttgatggggttctttgtttttttcttgtatatttgtttgagttcattgtagattctggatattagccctttgtcagatgagtagattgcaaaaattttctcccattctgtaggttgcctgttcactctgatggtagtttcttttgttgtacagaagctctttagtttaattagatcccatatttctaccatttttcattaaacattacaagttgttctcttgtgttcttattttttctgtaaacataattttaatggcagagatattctgctttatatctttccctattttatgtatatagaattataaagtttttaaaaatgtaatcatcattatttatagtttaataatcagtttcatcttgcagtgtatcattttcctcgtcattaaatattttacgtatcatttttaa

SEQ ID NO: 51 - DNA, 5690 bp, Homo sapiens
  source 1..5690: mol_type = other DNA
atttaagcgattttttttccctccttcatctccgggcctcggataagatgacggcttgggtgatgcacgaaataacgcacgtgattgattagacctggcttggcttggctagggaacgatccaggcgcgctggagaccccgcgtgaagatgaaatgacggctgccttggagttttcataagaaattgtccctggaggtgttggatgatcacagcttccttggagcattgcagttgctggaatccagtttcaggattaagggagggctgcctccttgcaatgggctgccaagaaaacggctgtgcttgttcttaacctcaggctctgtctgtgatcagtctgagagtctctcccaggtctactgctccctggaaagccctatctctctgcaggctcgcctctgggctttgtctccttggagccacatcactgggacagctgtggatgtggatgcagatttgaaccatgtcacggccccagggactgctatggcttcctttgttgttcaccccggtctgcgtcatgttaaactccaatgtcctcctgtggttaactgctcttgccatcaagttcaccctcattgacagccaagcacagtatccagttgtcaacacaaattatggcaaaatccggggcctaagaacaccgttacccaatgagatcttgggtccagtggagcagtacttaggggtcccctatgcctcaccccccactggagagaggcggtttcagcccccagaacccccgtcctcctggactggcatccgaaatactactcagtttgctgctgtgtgcccccagcacctggatgagagatccttactgcatgacatgctgcccatctggtttaccgccaatttggatactttgatgacctatgttcaagatcaaaatgaagactgcctttacttaaacatctacgtgcccacggaagatgatattcatgatcagaacagtaagaagcccgtcatggtctatatccatgggggatcttacatggagggcaccggcaacatgattgacggcagcattttggcaagctacggaaacgtcatcgtgatcaccattaactaccgtctgggaatactagggtttttaagtaccggtgaccaggcagcaaaaggcaactatgggctcctggatcagattcaagcactgcggtggattgaggagaatgtgggagcctttggcggggaccccaagagagtgaccatctttggctcgggggctggggcctcctgtgtcagcctgttgaccctgtcccactactcagaaggtctcttccagaaggccatcattcagagcggcaccgccctgtccagctgggcagtgaactaccagccggccaagtacactcggatattggcagacaaggtcggctgcaacatgctggacaccacggacatggtagaatgcctgcggaacaagaactacaaggagctcatccagcagaccatcaccccggccacctaccacatagccttcgggccggtgatcgacggcgacgtcatcccagacgacccccagatcctgatggagcaaggcgagttcctcaactacgacatcatgctgggcgtcaaccaaggggaaggcctgaagttcgtggacggcatcgtggataacgaggacggtgtgacgcccaacgactttgacttctccgtgtccaacttcgtggacaacctttacggctaccctgaagggaaagacactttgcgggagactatcaagttcatgtacacagactgggccgataaggaaaacccggagacgcggcggaaaaccctggtggctctctttactgaccaccagtgggtggcccccgccgtggccaccgccgacctgcacgcgcagtacggctcccccacctacttctatgccttctatcatcactgccaaagcgaaatgaagcccagctgggcagattcggcccatggtgatgaggtcccctatgtcttcggcatccccatgatcggtcccaccgagctcttcagttgtaacttttccaagaacgacgtcatgctcagcgccgtggtcatgacctactggacgaacttcgccaaaactggtgatccaaatcaaccagttcctcaggataccaagttcattcacacaaaacccaaccgctttgaagaagtggcctggtccaagtataatcccaaagaccagctctatctgcatattggcttgaaacccagagtgagagatcactaccgggcaacgaaagtggctttctggttggaactcgttcctcatttgcacaacttgaacgagatattccagtatgtttcaacaaccacaaaggttcctccaccagacatgacatcatttccctatggcacccggcgatctcccgccaagatatggccaaccaccaaacgcccagcaatcactcctgccaacaatcccaaacactctaaggaccctcacaaaacagggcctgaggacacaactgtcctcattgaaaccaaacgagattattccaccgaattaagtgtcaccattgccgtcggggcgtcgctcctcttcctcaacatcttagcttttgcggcgctgtactacaaaaaggacaagaggcgccatgagactcacaggcgccccagtccccagagaaacaccacaaatgatatcgctcacatccagaacgaagagatcatgtctctgcagatgaagcagctggaacacgatcacgagtgtgagtcgctgcaggcacacgacacactgaggctcacctgcccgccagactacaccctcacgctgcgccggtcgccagatgacatcccacttatgacgccaaacaccatcaccatgattccaaacacactgacggggatgcagcctttgcacacttttaacaccttcagtggaggacaaaacagtacaaatttaccccacggacattccaccactagagtatagctttgccctatttcccttcctatccctctgccctacccgctcagcaacatagaagagggaaggaaagagagaaggaaagagagagagaaagaaagtctccagaccaggaatgtttttgtcccactgacttaagacaaaaatgcaaaaaggcagtcatcccatcccggcagacccttatcgttggtgttttccagtattacaagatcaacttctgaccctgtgaaatgtgagaagtacacatttctgttaaaataactgctttaagatctctaccactccaatcgatgtttagtgtgataggacatcaccatttcaaggccccgggtgtttccaacgtcatggaagcagctgacacttctgaaactcagccaaggacacttgatattttttaattacaatggaagtttaaacatttctttctgtgccacacaatggatggctctccttaagtgaagaaagagtcaatgagattttgcccagcacatggagctgtaatccagagagaaggaaacgtagaaatttattattaaaagaatggactgtgcagcgaaatctgtacggttctgtgcaaagaggtgttttgccagcctgaactatatttaagagactttgtaaaaaagaaaaatgtatatagctgtgagtttaaacaaaaaccacaaacagacaaacaagaaaaaaagcttttattggtgttttcactttgaaagagcttttagcaaggttgtgcttttcattgtgctctgtacgtatataaatatatatatatatacacacacacacacacattagtcatatcacctctgtttcctccccaacaaaagaggcttttcttcttaattacttgtggtaaacaaagacatgggattttcttacatgagattctcatttgtaggaggatgtgatgtcccacagaagacccagacggtctgtgtggcctatttcccccgtcaggttgcacaggtgcatgcaagagcattcttaggagaccactgttttgaaaaacttttgacttgtacgtgttagccttcatgaaattgcagtacagagatgggtccccaaagtggagtgtatttacagcttgttaaattagagacatgcacacacaaagaatcagtagggagaaacaaaaatacaagtcccgttctgtagctctggccctttgaatatgtttaggaagagttgcttcccatttcagggccctgccaaaaaaagaagaaagcttgcctttggtggggctatgccccttggagtaaatacggctctgtgttccctagcagctgcgggagggtttggccgatgaagtacctgctcagcttagctaatcagattgaaggaagacatgtgtctttcctttttgtttaagcactcggtcccttatttatcagtaagcaggtttttaaaaatcttttatatcatttatgggatcaaacatatgattgtctgaaaacatcactttttgtggatttgtgtatccggtcaccaaacggtgaatattatagaagaatgggggaagaaaggatagaatattaaaactgctttgcatgggttttctgggaaattaggataacttcactgagaagacattgaatggaaattattcacccattttaaattggtgacctagggatcagagatttgtctttccaacagcttgtcattttttcatttctcttctcatttttcaggaaagttttgagtgttataaggtggaaggaaacatagtagcaatggatacttttttgaaaaattattgcattaccaagaaacagtagccaaagatatttgaagatcatgttcctcggctccattgtgggttattctagaaatccagtcttaaatctctccgctaaagtggacattccccataaaaattgtccagctgcctggctcttttgcaataacaacctttgattactgaatccctacactcaaactatagtgatatatcagtgtttgagagtgacctctagaaaaaagaaaagtgtttttagaaatgcgtacaagtcacccccaaatcctattgcttatcttgggttaaatttgagagtgattctctgtatataaatatgtgaaatattattatctcaacttagcacacgtgaagcaacatttctttcctacagagaggtgtcatggtaagatttcattccgaattcattgtttcatagagctatgatcaggccatttctgcaagcaatgtatgaccccacctgagcaaccacaaataggctctctgtgaaactacaaaggaagttatgtgtggcatccatgttggtttcgtctgtctgtaatgtgaattccagtatttgtttagtatttccagttgtctcctgctagcaatatgtacagtaacgcgtcaggcttgtgacatttgaataaggaaaaacagagttcctgttaagtgaataactttagcttttacaggggattatgatcaaaagtgattttagtacatcttaaatgatatcttatttctacatggaaagaagttatagaatcttcatagagttctatgagaaaaaatatacttgctatctataaaaaagagaaaaaagaaaaaaaatgagaaaaaagtaagaaaaaaaaaaatcctgtcctaggcttttactcttgatcttcaaaggcacgcagggtttaatggttccttgggttattattttgcagttttgttttttattttgccttaagtaatgatagaagatatatatggccggacacatatgtataaacttttcagcagcatttttaataataaaatatcacagtattttctaa

SEQ ID NO: 52 - DNA, 7796 bp, Homo sapiens
  source 1..7796: mol_type = other DNA
ggggccggggggcggagccttgcgggctggagcgaaagaatgcgggggctgagcgcagaagcggctcgaggctggaagaggatcttgggcgccgccagtctctctctgttgcccaagctggagtgcagtggcacagtcttggctcactgcaacctccacctcctgggtgcaagcgattctcgtgtctcagcctctcaagtagctgggattacagtctttagcaccagttggtgtaggagttgagacctacttcacagtagttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaaaaaaaaaaaaaaaaaa

SEQ ID NO: 53 - DNA, 4372 bp, Homo sapiens
  source 1..4372: mol_type = other DNA
ccctttcgcttccacgtccaaacccctttaagaaggatgaatgggcaggatgagttagactccttcgctgtatcgtctactgattcttaaaatgtgacaaatctgattggacgacttacatggcttctggagttacagtgaatgatgaagtcatcaaagtttttaatgatatgaaagtaaggaaatcttctacacaagaggagatcaaaaagagaaagaaagcagttctcttctgtttaagcgatgacaaaagacaaataattgtagaggaagcaaagcagatcttggtgggtgacattggtgatactgtagaggacccctacacatcttttgtgaagttgctacctctgaatgattgccgatatgctttgtacgatgccacatacgaaacaaaagagtctaagaaagaagacctagtatttatattctgggctcctgaaagtgcacctttaaaaagcaagatgatttatgctagctctaaagatgccattaaaaagaaatttacaggtattaaacatgagtggcaagtaaatggcttggatgatattaaggaccgttcgacacttggagagaaattgggaggcaatgtagtagtttcacttgaaggaaaaccattataaaatgacagtcaagtgccatctggatcttaaggagcttccatttctccagctcagtccattggaatagtattaggttttggttttttgttgtatttccccctttccactgggcccttccaacacaatgaatgaaggaaatatcatttatttaagcagcctatcagtgattgccattagactgttgaatactgttacttttatatagaacccaaggaatgccttcctgtcatattttagccaaaacaactggttatatgcctcccttgcagcaagcactacaatgtatgtgatcgtcaatgtgaatagcttagaatactgcaaaggataagctaattgaatgccttgaaagtattatccactggtcagatggtcaacttttttcagtattatttatagttggcacttgattgcagttctgtgaggcttgagcattcatacacctcacctgccttggcaagcctattttagtgatatggcagcacggatataacactatgcattaaaagcactttttgtaataagtttaatatcctaaaaggaatgccaattaagttttgttaactgtgtcatcaacttatcctagtacctcagtgttcattcctgttacctgcatatcttcttaaaagaaatagctgttattaatgcctttttgttttccattgagtgtacactactgaataagtgtaggagttttatgtttaccatgtgagtcctgcaacactaaagatattttgaatatcagtcatgatggcaatttctgtataaaagagccttaaatggaacattgttttgagatcaaactccccaccctcacaaaaatggccacgttgcaataaaaattgtggcatattacagaacgttgccttgttttccttggaaattttgcaaaatgttatgtgaaacaacttctagggtaaaaacagctattactaatctctgcactggtcatttgagaattttttttgtacagcattcatgtgtgatattttccagatttgttggatctatttggtttaaaaagtattctatcttaaggccaactaatataaaataccattgttaaagaatggtacttttataaacattagtgtatttatttcctatgtgttaatatgaagatcagaaattattttttgcactttggcataaatacttttcaatatctgatttgttctctggataaattagcatagttatttttttattcacatttacatttctaagtagttgtatagtagaagcaggaagctcttattgcttatttggtcgtaatgaaaataatttgtaaaatgtcctttaaaagtttaatgatacttctgatgtttcggaacagtcatttcacctactatttctgaatatattttgcaaattgaattggaataggaattgataatagcagtcttaaacattagtagtgggatttggctatggtccagactgtgctccttatagagaatttgatctgctcagtgtgagcggtttgctgttagccagggctatttatggcaaacacatgcttttgtatcttgtcatagttatccacaaatggcaaaactggacttgattctactggtatgcaaaacaggcatgctagtaagcagtcagtcgtggctcagaacttaaccccatagctcagaggaatgcttttagcagaaaacaggaaagaaaatatcccttaaaaattttttttgaatgtgtggaagtaattttagtataattagattttttccatatttttgaaagatttttcagatgtgaacattaaaaatagggattaaatgtctaggcttccatttaaaattatatgaatggtttgggatctttttgcactgagcaattttatttcaggcttccagctgtccctgtgagttatcctggacatttcgatggtttttggtaaggccaaactctgataagcaaaacagagaatactgacgtatacttaaccatatgtgtaactgatacttggcaccatggaatttttcattgagttatttcctcattcttttaaaaaataagggactataaatcagttatgtagtatcttttgtttttgtagctgattccttaactttcttgtatgcctctagtaatttcagagattaaatattgctttaaactgtgatactttgatttgctagattgacaaaactgatactaatataattaagttcatctttgaaatacatctttgtgcgtagagccaaaaaaagagataaaattaataatagttcacttgttatttgagattaatttggcatttgaaatgatcattttattttacaatcatttataatgaatcaatgttccagttagctttaaaaggtatacggtgctaattagtaaaatattgaaggcaatattttactgctagcttgcaaagttatgagagtttaaaaaataaaatatatgaaaatatgtaaagctgttgagatgtgtttacttatacttcagaacattaaaagtttaaaaactggtatttcagatcaaagaggcatcattatctttattctgtttttcaggattttagttggtaatactttctcatttatcaataacaattttctttaaaaactgagtgttacagcaattaattttagcattttcagacaaatgggaacagttggcatgtgtcccaaactggctatcagctgttgttttccatcattatctaaaatagtgtggccagcattgtgtattgaaatgtgcctttttcgtacattggaagagaagcctcttactgggtttgagtttctctgatacagaacatttgtagcagctaatttatggaatctggcaaataagctttgggaggaaattcttttaaaagtgttttcttgttaaggaataatctcaacaatggtaaatcatcaagaggttgagaagactcttaggtttgccactcagaaaaaacagtctcattttgctcttcataggtcctgtttttgcttgcacatttaatagtaggtggagatgcaaaacaacgtgaatgttcatttttttatgaatagtattaaagctactagaggtgggcaggcaggtcccagtaaatgttgcctttttttttttttttttttgagacagggtctcactctgttgcccaggctggagtgcagtgtcaaaatttcagctcacttcaaactcaaccttcccagctcagatgatcctcccacctcagcctcccaagtagctgggactacaggcatgcaccaccatgcctaattttttttttttttttttttgtatctcttttgtagagactgagtttggccatgttgcctaggctgatctccaactcctgggctcaagctgtcctctgacctccgcctcccaaaagtgctaggattgcatgcatgagccaccacttcaaaagctcaaatactgcttttgaagacagcatgagtttatggatgttctgtctcctttcagcccataaagaagtctgaggcttttcttttgctctggaacatccaactaattgctttatttccatgtagaagtagaggaatcatccctccacttaaatttcctctcataatgcaatttggcaaattaaatctctaaatctcagttttttaacccatagtatgggaacggtaatatctgtttaccatgtttcctgagtactaaatatggaaatggtttttgaaaacaggaaaatgctatgtaaatgcaataatctgtttaaactattcattcttaattactgtatgtaagtagataaatattaaatgtttttgttaaaagatgta

SEQ ID NO: 54 - DNA, 6685 bp, Homo sapiens
  source 1..6685: mol_type = other DNA
atggcatgctgtcacaaagtaatgctgctgctggacaccgcgggcggcgccgcccgccacagccgggtccggcgggccgccctgcgcctcctcacctatctgagttgccgattcggcctggccagggtccactgggccttcaagttctttgactcgcagggggcgcggagccggccgtcccgcgtgtctgacttccgcgagctggggtcccgctcgtgggaggactttgaggaggagctggaggccaggctcgaggatcgcgcccacctgcccggcccggcgcccagggccacccacacgcacggcgccctgatggagacgctgctagactaccagtgggaccggcccgagatcacgtcgcccacgaagccgatcctgcggagcagcgggaggagactgctggacgtggagagcgaggccaaggaggccgaggccgcgctcgggggcttggtgaacgccgtcttcctcctggccccctgtccgcactcgcagagggagctgctgcagttcgtgtctgggtgcgaggcccaggcccagcgcctgccgcccacccctaagcaggtgatggagaagttgttgcccaagagagtccgggaagtcatggtcgcccgaaaaatcaccttctactgggtggataccaccgaatggtctaagttgtgggaatccccagaccaccttggatactggactgtttgtgaactgctccaccacggaggtggcactgtcttgccatctgaatctttcagctgggattttgctcaagctggggaaatgctgctcaggagtggaataaagctgtcaagtgaacctcatctttctccgtggatttcaatgctgccaactgatgccactttaaaccgtttgctctacaattctcctgagtatgaggcctcgtttccacgaatggaaggaatgttatttctccctgttgaaggcaaagagattcaagaaacatggacagtcaccctagagcccttggccatgcatcagagacattttcagaaaccagtcagaatttttctaaaaggctcagtggcccagtggtctctcccaacgagcagcactttgggcactgacagctggatgctaggaagtccagaggagagcacagcaactcaaaggctgttatttcagcagttggtaagcaggctgactgctgaagagttacacctggttgctgatgtggaccctggtgaaggccggccccccatcactggagttatttccccactctctgccagtgctatgatcctcactgtgtgccgcaccaaggaggctgaatttcaacgacatgttctccaaacagctgtggctgacagcccccgggacacagcttcccttttctcagatgttgtggatagtatattgaatcagactcatgattcgcttgcagatactgcttctgctgcttctcctgttccagagtgggcccagcaggagcttggccacaccactccctggagtccagctgttgtggaaaagtggtttcctttctgtaacatcagtggtgccagttccgatttgatggagtcatttgggttactacaggctgcctcagctaataaggaagagtcttccaaaactgaaggcgaattaatacattgccttgccgagctctaccagagaaaatctcgtgaagaatccactatagctcatcaagaagacagcaaaaagaaacgaggggtccctcgtactccagtgagacagaagatgaataccatgtgccgttccttaaagatgttgaatgtcgcaaggctgaatgtgaaggcccagaagttacatccagatggcagtccggatgtggctggggagaaaggaatccaaaagatacctagtgggagaacagtggataaattggaagacagaggaagaacactaagaagttctaaacctaaagattttaaaactgaggaagagctgctatcatatatacgtgaaaattaccaaaagactgtggccacaggagaaatcatgttgtatgcatgtgctcgaaacatgatctcaaccgttaaaatgttcctaaaatcaaaaggcaccaaggaattagaagtgaactgcctgaatcaagtaaaaagtagtctcttaaaaactagtaaaagtcttcgacagaatctaggaaaaaaactggataaggaagacaaagttagagagtgccagcttcaggtatttcttcgtttggagatgtgtctgcaatgcccttcaataaatgaaagtacagatgatatggaacaagtagtggaggaggtgacagatttgctgcgcatggtgtgtttaactgaggattcagcgtacctagcagagtttctggaggaaattttgagattgtatattgactctatcccaaagacacttggaaatctttacaacagcctagggtttgtgattcctcagaagctggctggtgtccttcctacagattttttcagtgatgactccatgacacaagagaacaaatcaccacttctttctgtgccttttttgtcaagtgctcgtagatcagtgtcaggcagccctgaatctgatgaactgcaggaacttcgtaccagatcagccaagaagagaaggaaaaatgcattaataagacataaaagcattgctgaggtttcacagaatcttcgacaaattgaaattcctaaagtgtcaaagagagctacgaaaaaagagaactctcaccctgctcctcagcagccttcccagccagtgaaagatacagtgcaagaagtgaccaaagttcgaagaaatcttttcaaccaggaattgctttccccttcaaagagatcactaaagcgggggttgcctagaagccattctgtgtcagctgtggatggtctagaggataaacttgacaacttcaagaagaacaaaggttatcacaaactgctgactaagagtgtggccgagactccagtgcataagcagatctccaaaaggctgctgcacagacaaatcaagggcaggtcctctgatcctggtcctgatattggtgttgttgaagagtcccctgaaaaaggagatgaaataagtctgagacgaagtcctcgaatcaagcagttgtcatttagcaggacacattctgcctccttctattctgtgtctcagccgaagtctcgaagtgtgcaaagagtccactctttccagcaagataagtcagaccaaagagaaaattctccagtccaaagtattcggtctcccaagagtcttctttttggggcaatgtctgagatgatcagcccctcagaaaagggttcagctcgaatgaaaaagcgttcaagaaacactttggattcggaggtacctgcagcttaccagactcccaagaagagtcaccagaaatctctgagcttttctaaaactacaccaagaaggatctctcatacaccacaaactccgttgtatactccagaaaggctgcagaagtcccctgcaaaaatgacccctacaaagcaggcagcttttaaggagtccttaaaagactcctcctcacccggccatgactcaccattggattcaaaaatcactcctcaaaaacgacatacccaggcaggagaaggtacctctcttgaaacgaagacaccaagaactcctaagaggcaaggtactcagccgcctgggtttttgccaaactgtacttggccacattcagtgaattccagtccagaaagcccctcctgtccagcccctccaacttcatcgactgcccagcccaggagagagtgtctcactcccatcagagaccctctcagaacacctccgagagcagcagccttcatgggcacgcctcagaatcaaacacaccaacagccccatgtcctcagagctgctcgggcagaggaaccagcccagaaactaaaggataaagctatcaaaactccaaaaagaccagggaattcaactgtgacttcttccccacctgttacgccaaagaaactgtttacctctcctttatgtgatgtctccaagaagagtccatttaggaaatctaaaatagagtgtccttccccaggagaactggatcagaaagagccccagatgtcacccagcgtagctgcatctctctcctgccctgttccctcaactccccctgaactctcacagagagctacattggacaccgtccctcctccacccccttctaaagttgggaaacggtgtagaaagacctctgatcccagaaggagcatcgtggagtgtcagcctgatgcctccgctactcctggggttggcacagctgacagcccagctgcccccacagactctagagatgaccagaagggactgagcctctctcctcagtctcctcctgaaagacggggctacccaggccctggtctcaggagtgattggcatgcatcctctcctctgctcattacaagtgacacagagcatgtcactctcctcagtgaagccgaacaccatggcattggtgacttgaaaagtaacgtcttatcagtggaagagggtgaggggctaaggacagcagatgctgagaagtcttctctgtctcaccctgggattcccccatctcctccttcctgtgggcctggctctcctctgatgccttcccgtgacgtgcactgtaccacagatgggagacagtgccaggcttcggcacaactagacaacctgccagcatcagcttggcattccacagactctgccagcccacagacctatgaggttgagctggagatgcaagcttctggccttcccaaacttcgaattaagaagatagaccccagctcttcattagaggctgagcccctcagcaaggaggagagctctctgggagaagagagcttcctccctgctctcagcatgcccagggccagcaggtccttaagcaaacctgaacccacctatgtgtcacccccctgcccccgcctctcccacagcacacctggcaagagcagggggcaaacctacatctgccaggcctgtacccccacccacggcccttctagtaccccctctccatttcaaacagatggggttccttggacaccatcccccaagcacagtgggaagacaactccagacataattaaagactggcccaggaggaagagggcggtgggctgtggcgccggctcctcttccgggaggggcgaggtcggtgcagaccttcctgggagcctgtcactgcttgagtcagagggcaaggaccacggccttgaactcagcatccacaggacgcccatcttggaggattttgagctcgagggagtgtgccagctcccagaccagtcgcctcccaggaacagcatgcctaaggccgaggaagcctcttcctggggacagtttgggttgagttccaggaagagagtcctgttggccaaggaagaagctgaccgtggagccaaaaggatctgtgacctgagagaagattcagaagttagtaagagtaaagaggggtctccaagttggagtgcatggcagctaccctccacgggagacgaagaggtgtttgtttccggctccaccccacctcccagctgtgccgtgcggagctgcctctctgccagtgccctccaggctctgacccagtctccgctgctgttccaggggaaaacaccttcctctcagagcaaagaccccagagatgaggatgtggatgttcttccctccactgtagaagactctcctttcagtcgcgctttctccaggaggcgccccatcagcagaacttatacacggaagaagctcatgggaacctggctggaggacttatagccacaaacattactgagcccaaaagatcaaggagtcagccaggaccctgtggacataaagaagttggatgcctggtcccaagcctcttttgccatggtcagtgttcagattgccattagaatgccttagggttttctaattccccttatggatccaatccatctcctggccctgccccttgttggggaagttgcaggaggagaggtggatggcaatgtgattggtgctataactcaggcagcctgggagtcaggaacccagacaaggaatcccattccagcctcaccccaaccatgaccttggcaagtcagggggccactctgcctcatttatgcaaatggagaaaggcgccctccctggggtcccttgagctgctgtaaggctgggctgctgcgacacaggcagcgctttgtaaactgtgaagccatatacgtgaaactgaagagtgcattgggcagtggaagctattttttgccttccctgtgtaacagtaaaatcatctctagtgactgagcactcagtacatttttgtttaatgttgggcctgaggttaactgtgaccatggtccagcttgagtggcttctggagcagccacattttcaaggactgtccaagagccagccagttcagggctcaggcctcacccattgcccactcctggggagaccatcacctggctcatcgtttccaccaagagtgccccacaggagtgccccacagacccgctggaccagcctgctgcgggtcctggccaggggtctggctaacggtgagggctgactctgaactgtctctcagtctccagaaagtgttcaagcctgttgtgttcccaaatctgattcctcctattgtcttgtaaatcaaactctaagtgaaaacttcccatttgtcccttcaaagatttttttttattaaatggttttttaagatcctaaaaaaaaaaaaaaaaaaaaa

SEQ ID NO: 55 - DNA, 3222 bp, Homo sapiens; identical to SEQ ID NO: 49
  source 1..3222: mol_type = other DNA

SEQ ID NO: 56 - DNA, 1545 bp, Homo sapiens
  source 1..1545: mol_type = other DNA
attattttagcttcctgagattcagaggccaggaactgtgcagagatctgtggggattctcacaacttccatttctggtgaacagctgaggtcagagaggagttggtccaggcgcaatgttcagcgtatttgaggaaatcacaagaattgtagttaaggagatggatgctggaggggatatgattgccgttagaagccttgttgatgctgatagattccgctgcttccatctggtgggggagaagagaactttctttggatgccggcactacacaacaggcctcaccctgatggacattctggacacagatggggacaagtggttagatgaactggattctgggctccaaggtcaaaaggctgagtttcaaattctggataatgtagactcaacgggagagttgatagtgagattacccaaagaaataacaatttcaggcagtttccagggcttccaccatcagaaaatcaagatatcggagaaccggatatcccagcagtatctggctacccttgaaaacaggaagctgaagagggaactacccttttcattccgatcaattaatacgagagaaaacctgtatctggtgacagaaactctggagacggtaaaggaggaaaccctgaaaagcgaccggcaatataaattttggagccagatctctcagggccatctcagctataaacacaagggccaaagggaagtgaccatccccccaaatcgggtcctgagctatcgagtaaagcagcttgtcttccccaacaaggagacgatgaagaaggatggtgcttcatcctgtttaggaaagtctttgggttcggaggattccagaaacatgaaggagaagttggaggacatggagagtgtcctcaaggacctgacagaggagaagagaaaagatgtgctaaactccctcgctaagtgcctcggcaaggaggatattcggcaggatctagagcaaagagtatctgaggtcctgatttccggggagctacacatggaggacccagacaagcctctcctaagcagcctttttaatgctgctggggtcttggtagaagcgcgtgcaaaagccattctggacttcctggatgccctgctagagctgtctgaagagcagcagtttgtggctgaggccctggagaaggggacccttcctctgttgaaggaccaggtgaaatctgtcatggagcagaactgggatgagctggccagcagtcctcctgacatggactatgaccctgaggcacgaattctctgtgcgctgtatgttgttgtctctatcctgctggagctggctgaggggcctacctctgtctcttcctaactacaaaagccctttctccccacaagcctctgggttttccctttaccagtctgtcctcactgccatcgccactaccatcctgtcaccagtgggacctctttaaaacaagcagccaaccattctttgatgtatcccattcgctccatgttaacatccaaaaccagcctggatttcatacatggacttctgattaaaagtggcaggttgtgcatgttaa

SEQ ID NO: 57 - DNA, 7169 bp, Homo sapiens
  source 1..7169: mol_type = other DNA
attctttggaatactactgctagaagtctgacttaagacccagcttatgggccacatggcacccagctgcttctgcagagaaggcaggccactgatgggtacagcaaagtgtggtgctgctggccaagccaaagacccgtgtaggatgactgggcctctgccccttgtgggtgttgccactgtgcttgagtgcctggtgaagaatgtgatgggatcactagcatgtctgcggagagcggccctgggacgagattgagaaatctgccagtaatgggggatggactagaaacttcccaaatgtctacaacacaggcccaggcccaaccccagccagccaacgcagccagcaccaaccccccgcccccagagacctccaaccctaacaagcccaagaggcagaccaaccaactgcaatacctgctcagagtggtgctcaagacactatggaaacaccagtttgcatggcctttccagcagcctgtggatgccgtcaagctgaacctccctgattactataagatcattaaaacgcctatggatatgggaacaataaagaagcgcttggaaaacaactattactggaatgctcaggaatgtatccaggacttcaacactatgtttacaaattgttacatctacaacaagcctggagatgacatagtcttaatggcagaagctctggaaaagctcttcttgcaaaaaataaatgagctacccacagaagaaaccgagatcatgatagtccaggcaaaaggaagaggacgtgggaggaaagaaacagggacagcaaaacctggcgtttccacggtaccaaacacaactcaagcatcgactcctccgcagacccagacccctcagccgaatcctcctcctgtgcaggccacgcctcaccccttccctgccgtcaccccggacctcatcgtccagacccctgtcatgacagtggtgcctccccagccactgcagacgcccccgccagtgcccccccagccacaacccccacccgctccagctccccagcccgtacagagccacccacccatcatcgcggccaccccacagcctgtgaagacaaagaagggagtgaagaggaaagcagacaccaccacccccaccaccattgaccccattcacgagccaccctcgctgcccccggagcccaagaccaccaagctgggccagcggcgggagagcagccggcctgtgaaacctccaaagaaggacgtgcccgactctcagcagcacccagcaccagagaagagcagcaaggtctcggagcagctcaagtgctgcagcggcatcctcaaggagatgtttgccaagaagcacgccgcctacgcctggcccttctacaagcctgtggacgtggaggcactgggcctacacgactactgtgacatcatcaagcaccccatggacatgagcacaatcaagtctaaactggaggcccgtgagtaccgtgatgctcaggagtttggtgctgacgtccgattgatgttctccaactgctataagtacaaccctcctgaccatgaggtggtggccatggcccgcaagctccaggatgtgttcgaaatgcgctttgccaagatgccggacgagcctgaggagccagtggtggccgtgtcctccccggcagtgccccctcccaccaaggttgtggccccgccctcatccagcgacagcagcagcgatagctcctcggacagtgacagttcgactgatgactctgaggaggagcgagcccagcggctggctgagctccaggagcagctcaaagccgtgcacgagcagcttgcagccctctctcagccccagcagaacaaaccaaagaaaaaggagaaagacaagaaggaaaagaaaaaagaaaagcacaaaaggaaagaggaagtggaagagaataaaaaaagcaaagccaaggaacctcctcctaaaaagacgaagaaaaataatagcagcaacagcaatgtgagcaagaaggagccagcgcccatgaagagcaagccccctcccacgtatgagtcggaggaagaggacaagtgcaagcctatgtcctatgaggagaagcggcagctcagcttggacatcaacaagctccccggcgagaagctgggccgcgtggtgcacatcatccagtcacgggagccctccctgaagaattccaaccccgacgagattgaaatcgactttgagaccctgaagccgtccacactgcgtgagctggagcgctatgtcacctcctgtttgcggaagaaaaggaaacctcaagctgagaaagttgatgtgattgccggctcctccaagatgaagggcttctcgtcctcagagtcggagagctccagtgagtccagctcctctgacagcgaagactccgaaacagagatggctccgaagtcaaaaaagaaggggcaccccgggagggagcagaagaagcaccatcatcaccaccatcagcagatgcagcaggccccggctcctgtgccccagcagccgcccccgcctccccagcagcccccaccgcctccacctccgcagcagcaacagcagccgccacccccgcctcccccaccctccatgccgcagcaggcagccccggcgatgaagtcctcgcccccacccttcattgccacccaggtgcccgtcctggagccccagctcccaggcagcgtctttgaccccatcggccacttcacccagcccatcctgcacctgccgcagcctgagctgccccctcacctgccccagccgcctgagcacagcactccaccccatctcaaccagcacgcagtggtctctcctccagctttgcacaacgcactaccccagcagccatcacggcccagcaaccgagccgctgccctgcctcccaagcccgcccggcccccagccgtgtcaccagccttgacccaaacacccctgctcccacagccccccatggcccaacccccccaagtgctgctggaggatgaagagccacctgccccacccctcacctccatgcagatgcagctgtacctgcagcagctgcagaaggtgcagccccctacgccgctactcccttccgtgaaggtgcagtcccagcccccaccccccctgccgcccccaccccacccctctgtgcagcagcagctgcagcagcagccgccaccacccccaccaccccagccccagcctccaccccagcagcagcatcagccccctccacggcccgtgcacttgcagcccatgcagttttccacccacatccaacagcccccgccaccccagggccagcagcccccccatccgcccccaggccagcagccacccccgccgcagcctgccaagcctcagcaagtcatccagcaccaccattcaccccggcaccacaagtcggacccctactcaaccggtcacctccgcgaagccccctccccgcttatgatacattccccccagatgtcacagttccagagcctgacccaccagtctccaccccagcaaaacgtccagcctaagaaacaggagctgcgtgctgcctccgtggtccagccccagcccctcgtggtggtgaaggaggagaagatccactcacccatcatccgcagcgagcccttcagcccctcgctgcggccggagccccccaagcacccggagagcatcaaggcccccgtccacctgccccagcggccggaaatgaagcctgtggatgtcgggaggcctgtgatccggcccccagagcagaacgcaccgccaccaggggcccctgacaaggacaaacagaaacaggagccgaagactccagttgcgcccaaaaaggacctgaaaatcaagaacatgggctcctgggccagcctagtgcagaagcatccgaccaccccctcctccacagccaagtcatccagcgacagcttcgagcagttccgccgcgccgctcgggagaaagaggagcgtgagaaggccctgaaggctcaggccgagcacgctgagaaggagaaggagcggctgcggcaggagcgcatgaggagccgagaggacgaggatgcgctggagcaggcccggcgggcccatgaggaggcacgtcggcgccaggagcagcagcagcagcagcgccaggagcaacagcagcagcagcaacagcaagcagctgcggtggctgccgccgccaccccacaggcccagagctcccagccccagtccatgctggaccagcagagggagttggcccggaagcgggagcaggagcgaagacgccgggaagccatggcagctaccattgacatgaatttccagagtgatctattgtcaatatttgaagaaaatcttttctgagcgcacctaggtggcttctgactttgattttctggcaaaacattgactttccatagtgttaggggcggtggtggaggtgggatcagcggccaggggatgcctcagggcctggccctcctgcatgctatgcccggggcaggcctgacgggcagctgaggattgcagagcctgtctgccttacggccagtcggacagacgtcccgccacccaccacccctcacaggacgtccgctcagcacacgccttgttacgagcaagtgccggctggacccaagccctgcatccccacatgcggggcagaggcccttctctccgccaaatgtctacacagtatacacaggacatcgttgctgccgccgtgactggttttctgtccccaagaacgtgacgttcgtgatgtcctgcccgccgggagtctttccccacaccccagccatcgccgcccgctcccaggaggccagggcaggcctgcgtgggctggaggcgggcgaggccggcccaccccctcgctggcactgactttgccttgaacagaccccccgaccctcccccacaagcctttaattgagagccgctctctgtaagtgtttgcttgtgcaaaagggaatagtgccgtggaggtgtgtgtgtccatggcatccggagcgaggcgactgtcctgcgtgggtagccctcggccggggagtgaggccaccaaccaaagtcagttccttcccacctgtgtttctgtttcgtttttttttttcttttttttctatatatattttttgttgaattctattttatttttaattctctcttctcctccagacacaatggcactgcttatctccgaaatggtgtgatcgtctcctcattgagcagcggctgccaccgcgctgtgggtagtgtgtgaccgtggctgtactgtatagtgaacatagttggcatatctttgtttgaagtttgttggtgactccaccaaactggtgtgaaaaaagaaaaaagctcaaaaaaatccacaaaaagacaaaacacacaaaaaaaatcctgcctatattttactcagtttcaaactttattagtctatttttaattataaaaccagaaagctacaatttcttttctttcccctccaccccccccccccccacccatttgttggcttttttgttttttaatgtcagatctgttgagttggtttttttggttttggtttttgtttttgtttttgttttttactgagaaaggaagggccaagggatgaggtgggaaccgggccctgggggcgccacagactaaggcagagactcccctacctggcgcccagccccaaccagctggccgctcctgcccatgcttttttttttttttttttttaatttttataattggagcccctggtgaggttacgcgtgccatgagaacccactctacaccacgacgctggtgcctcagtgttggccaaactctggagtcactgactggtttgactttcatacggtgaatatgcatttggtctgtactgatcatggaataaacacatctctctttttttaatgctggcgtctccctgacatttctttgtgaaccaactgttgcctaggctaggcccaggggaccccctggaccccagaccacctctgtacaggaactactgccagggattacctagcccctctcctgtgacctgtccctgtctgccctgggcgggagccacgcagactcatagcaaccaccccaagctgaagctgtgacgcagagcccggtacccatccttgtggaccctggctgaggtggagggtgtgctctagcagaaccctggccagactccagacagtattcttcccctccaccctactccatcctgtcccccctactccatcctgtccccccactcaccaaaggacttgggccacttctcccaccttgcctgcctcaacctaactcctcctttcatttaagctcagggttaaccagatactcttagatataagtctacatcccccaaaataggatcctcaccccccatgcacatacacacacattcctgtccaagaaagcccacaggtggctgctctgcctgtgtgtccacttgtgtatgtacatgccccagccacaaggcacgggtgacgcccaagaagagcccctaagatgtaagatacaagtatataatttatatgtatgcagagacaaactgattgaaacatttctagcactgtttattctcctacatcccctctttttgacctgaaaggtcctttattgtctttggatctgccaaacctccctgtgagggtgtggttcaggcatccagccagaggtccccccacttcttcgatgtccatgcccactcaggcactttctcatataccactgtgccacttcaagtgtgacgtatttaggttctttggggtttcttccatctcatcccacccactcatttttgttccttgttttgttcagacacttccccagcctgggtccaactgctgagctggatagttttctctgcagcttcctgctgaagatttaactcagccatggccttctccacacctccctgatgggtgtctctctacctgggttgtaattagaactgggatctatttaatttctccagccatttcccaacccttcccacagtactagaaatcttagtcctataccagagtaagaggtgtgtacaagcccccactgtactgtatgcacggatcgcttggccaataattatgtcagtgaatctgagacttgtattaaacactttagacatttgtagaagggaattcgtagacttttcacttacatacgaaaggtttttttttttttttgtgcagttctcattgcaaaaataaacatttgtactgagtataaagtta

SEQ ID NO: 58 - DNA, 2390 bp, Homo sapiens
  source 1..2390: mol_type = other DNA
gcagacaggaagacttctgaagaacaaatcagcctggtcaccagcttttcggaacagcagagacacagagggcagtcatgagtgaggtcaccaagaattccctggagaaaatccttccacagctgaaatgccatttcacctggaacttattcaaggaagacagtgtctcaagggatctagaagatagagtgtgtaaccagattgaatttttaaacactgagttcaaagctacaatgtacaacttgttggcctacataaaacacctagatggtaacaacgaggcagccctggaatgcttacggcaagctgaagagttaatccagcaagaacatgctgaccaagcagaaatcagaagtctagtcacttggggaaactacgcctgggtctactatcacttgggcagactctcagatgctcagatttatgtagataaggtgaaacaaacctgcaagaaattttcaaatccatacagtattgagtattctgaacttgactgtgaggaagggtggacacaactgaagtgtggaagaaatgaaagggcgaaggtgtgttttgagaaggctctggaagaaaagcccaacaacccagaattctcctctggactggcaattgcgatgtaccatctggataatcacccagagaaacagttctctactgatgttttgaagcaggccattgagctgagtcctgataaccaatacgtcaaggttctcttgggcctgaaactgcagaagatgaataaagaagctgaaggagagcagtttgttgaagaagccttggaaaagtctccttgccaaacagatgtcctccgcagtgcagccaaattttacagaagaaaaggtgacctagacaaagctattgaactgtttcaacgggtgttggaatccacaccaaacaatggctacctctatcaccagattgggtgctgctacaaggcaaaagtaagacaaatgcagaatacaggagaatctgaagctagtggaaataaagagatgattgaagcactaaagcaatatgctatggactattcgaataaagctcttgagaagggactgaatcctctgaatgcatactccgatctcgctgagttcctggagacggaatgttatcagacaccattcaataaggaagtccctgatgctgaaaagcaacaatcccatcagcgctactgcaaccttcagaaatataatgggaagtctgaagacactgctgtgcaacatggtttagagggtttgtccataagcaaaaaatcaactgacaaggaagagatcaaagaccaaccacagaatgtatctgaaaatctgcttccacaaaatgcaccaaattattggtatcttcaaggattaattcataagcagaatggagatctgctgcaagcagccaaatgttatgagaaggaactgggccgcctgctaagggatgccccttcaggcataggcagtattttcctgtcagcatctgagcttgaggatggtagtgaggaaatgggccagggcgcagtcagctccagtcccagagagctcctctctaactcagagcaactgaactgagacagaggaggaaaacagagcatcagaagcctgcagtggtggttgtgacgggtaggacgataggaagacagggggccccaacctgggattgctgagcagggaagctttgcatgttgctctaaggtacatttttaaagagttgttttttggccgggcgcagtggctcatgcctgtaatcccagcactttgggaggccgaggtgggcggatcacgaggtctggagtttgagaccatcctggctaacacagtgaaatcccgtctctactaaaaatacaaaaaattagccaggcgtggtggctggcacctgtagtcccagctacttgggaggctgaggcaggagaatggcgtgaacctggaaggaagaggttgcagtgagccaagattgcgcccctgcactccagcctgggcaacagagcaagactccatctcaaaaaaaaaaaaaaaaaaaaaaaagagttgttttctcatgttcattatagttcattacagttacatagtccgaaggtcttacaactaatcactggtagcaataaatgcttcaggcccacatgatgctgattagttctcagttttcattcagttcacaatataaccaccattcctgccctccctgccaagggtcataaatggtgactgcctaacaacaaaatttgcagtctcatctcattttcatccagacttctggaactcaaagattaacttttgactaaccctggaatatctcttatctcacttatagcttcaggcatgtatttatatgtattcttgatagcaataccataatcaatgtgtattcctgatagtaatgctacaataaatccaaacatttcaactctgtta

SEQ ID NO: 59 - DNA, 2410 bp, Homo sapiens
  source 1..2410: mol_type = other DNA
agcgcgagccccgccgccgccgagcatggacgaccccgactgcgactccacctgggaggaggacgaggaggatgcggaggacgcggaggacgaggactgcgaggacggcgaggccgccggcgcgagggacgcggacgcaggggacgaggacgaggagtcggaggagccgcgggcggcgcggcccagctcgttccagtccagaatgacagggtccagaaactggcgagccacgagggacatgtgtaggtatcggcacaactatccggatctggtggaacgagactgcaatggggacacgccaaacctgagtttctacagaaatgagatccgcttcctgcccaacggctgtttcattgaggacattcttcagaactggacggacaactatgacctccttgaggacaatcactcctacatccagtggctgtttcctctgcgagaaccaggagtgaactggcatgccaagcccctcacgctcagggaggtcgaggtgtttaaaagctcccaggagatccaggagcggcttgtccgggcctacgagctcatgctgggcttctacgggatccggctggaggaccgaggcacgggcacggtgggccgagcacagaactaccagaagcgcttccagaacctgaactggcgcagccacaacaacctccgcatcacacgcatcctcaagtcgctgggtgagctgggcctcgagcacttccaggcgccgctggtccgcttcttcctggaggagacgctggtgcggcgggagctgccgggggtgcggcagagtgccctggactacttcatgttcgccgtgcgctgccgacaccagcgccgccagctggtgcacttcgcctgggagcacttccggccccgctgcaagttcgtctgggggccccaagacaagctgcggaggttcaagcccagctctctgccccatccgctcgagggctccaggaaggtggaggaggaaggaagccccggggaccccgaccacgaggccagcacccagggtcggacctgtgggccagagcatagcaagggtgggggcagggtggacgaggggccccagccacggagcgtggagccccaggatgcgggacccctggagaggagccagggggatgaggcagggggccacggggaagataggccggagcccttaagccccaaagagagcaagaagaggaagctggagctgagccggcgggagcagccgcccacagagccaggccctcagagtgcctcagaggtggagaagatcgctctgaatttggaggggtgtgccctcagccagggcagcctcaggacggggacccaggaagtgggcggtcaggaccctggggaggcagtgcagccctgccgccaacccctgggagccagggtggccgacaaggtgaggaagcggaggaaggtggatgagggtgctggggacagtgctgcggtggccagtggtggtgcccagaccttggcccttgccgggtcccctgccccatcggggcaccccaaggctggacacagtgagaacggggttgaggaggacacagaaggtcgaacggggcccaaagaaggtacccctgggagcccatcggagaccccaggccccagcccagcaggacctgcaggggacgagccagccgagagcccatcggagaccccaggcccccgcccagcaggacctgcaggggacgagccagccgagagcccatcggagaccccaggcccccgcccggcaggacctgcaggggacgagccagccgagagcccatcggagaccccaggccccagcccggcaggacctacaagggatgagccagccgagagcccatcggagaccccaggcccccgcccggcaggacctgcaggggacgagccagccgagagcccatcggagaccccaggcccccgcccggcaggacctgcaggggacgagccagccgagagcccatcggagaccccaggccccagcccggcaggacctacaagggatgagccagccaaggcgggggaggcagcagagttgcaggacgcagaggtggagtcttctgccaagtctgggaagccttaaggaaaggagtgcccgtcggcgtcttggtcctcctgtccctgctgcaggggctggggcctccggagctgctgcgggctcccctcaggctctgcttcgtgacccgtgacccatgacccacagtgctggcctcctgtggggccactatagcagccaccagaagccgcgaggccctcagggaagcccaaggcctgcagaagcctcctggcctggctgtgtcttccccacccagctctcccctgcgcccctgtctttgtaaattgacccttctggagtggggggcggcgggcagggctgcttttcttagtctgataccaagcaaggccttttctgaataaattcatttgactttga

SEQ ID NO: 60 - DNA, 1206 bp, Homo sapiens
  source 1..1206: mol_type = other DNA
agtcgctatgcgtgtgcttgtgggtggcgggacaggcttcattgggacagccctaacccagctgctgaatgccagaggccacgaagtgacgttggtctcccgaaagcccgggcccggccggatcacgtgggatgagctcgctgcatcggggctgccgagctgcgatgccgccgtcaacctggccggagagaacatcctcaaccctctccgaagatggaatgaaaccttccaaaaagaggtaatcggcagccgcctagagaccacccaattgctggctaaagccatcaccaaagccccacaaccccccaaggcctgggtcttagtcacaggtgtagcttactaccagcccagtctgactgcggagtatgatgaagacagcccaggaggggactttgactttttctccaacctcgtaaccaaatgggaagctgcagccaggcttcctggagattctacacgccaggtggtggtgcgctcaggggttgtgctgggccgtgggggtggtgccatgggccacatgctgctgccctttcgcctgggcctggggggccccatcggctcaggccaccaattcttcccctggatacacatcggggacctggcaggaatcctgacccatgcccttgaagcaaaccacgtgcacggggtcctgaatggagtggctccatcctccgccactaatgctgagtttgcccagaccttgggtgctgccctgggccgccgagccttcatccctctccccagcgctgtggtgcaagctgtctttgggcgacagcgtgccatcatgctgctggagggccagaaggtgatcccacagcgaacactggccactggctaccagtattccttcccagagctaggggctgccttaaaggaaattgtagcctaagtaggtcgtggcaagggcctgaggcctgttcctcacaggcttccaggttaggcactgtgaataggctcagctcctctagagagctgaagccatctggttcttagattcctctcccagtcctctttcccattgttctgttgctccaccttattgtctcaaggccgtaatctcatcaggttgggacattaatcttttcaactccttgtaagatttcccagtttggtttctctacatgtcctgcagctgccccacttctcctttacgctgtgtagagaatgctctgcagtttaggcaataaaaataaattgtctcactaa

SEQ ID NO: 61 - DNA, 8601 bp, Homo sapiens
  source 1..8601: mol_type = other DNA
agttcccctttcccttgaaccgctcacttcacagcccttcgcccccgggaagaagaaacatttcccgaagcgcactcctcagccctccttccccacgcgctcgccctcccctccccctgcttttcttgggggaggggggctgtcgccttggattgaaggccattgatttgtatgtatttgtcccagcgctggaggctgccccagccgccgcgccggtgccgccgctgccagtggagttgcctccccgcttccctagggtggttcggctccaccaaacatgtcggctcctgtcgggccccggggccgcctggctcccatcccggcggcctctcagccgcctctgcagcccgagatgcctgacctcagccacctcacggaggaggagaggaaaatcatcctggccgtcatggataggcagaagaaagaagaggagaaggagcagtccgtgctcaaaaaactgcatcagcagtttgaaatgtataaagagcaggtaaagaagatgggagaagaatcacagcaacagcaagaacagaagggtgatgcgccaacctgtggtatctgccacaaaacaaagtttgctgatggatgtggccataactgttcatattgccaaacaaagttctgtgctcgttgtggaggtcgagtgtcattacgctcaaacaaggttatgtgggtatgtaatttgtgccgaaaacaacaagaaatcctcactaaatcaggagcatggttttataatagtggatctaatacaccacagcaacctgatcaaaaggttcttcgagggctaagaaatgaggaggcacctcaggagaagaaaccaaaactacatgagcagacccagttccaaggaccctcaggtgacttatctgtacctgcagtggagaaaagtcgatctcatgggctcacaagacagcattctattaaaaatgggtcaggcgtgaagcatcacattgccagtgacatagcttcagacaggaaaagaagcccatctgtgtccagagatcagaatagaagatacgaccaaagggaagaaagagaggaatattcacagtatgctacttcggataccgcaatgcctagatctccatcagattatgctgataggcgatctcaacatgaacctcagttttatgaagactctgatcatttaagttatagggactccaacaggagaagtcataggcattccaaagaatatattgtagatgatgaggatgtggaaagcagagatgaatacgaaaggcaaaggagagaggaagagtaccagtcacgctaccgaagtgatccgaatttggcccgttatccagtaaagccacaaccctatgaagaacaaatgcggatccatgctgaagtgtcccgagcacggcatgagagaaggcatagtgatgtttctttggcaaatgctgatctggaagattccaggatttctatgctaaggatggatcgaccatcaaggcaaagatctatatcagaacgtagagctgccatggaaaatcagcgatcttattcaatggaaagaactcgagaggctcagggaccaagttcttatgcacaaaggaccacaaaccatagtcctcctacccccaggaggagtccactacccatagatagaccagacttgaggcgtactgactcactacggaaacagcaccacttagatcctagctctgctgtaagaaaaacaaaacgggaaaaaatggaaacaatgttaaggaatgattctctcagttcagaccagtcagagtcagtgagacctccaccaccaaagcctcataaatcaaagaaaggcggtaaaatgcgccagatttcgttgagcagttcagaggaggaattggcttccacgcctgaatatacaagttgtgatgatgttgagattgaaagtgagagtgtaagtgaaaaaggagacatggattacaactggttggatcatacgtcttggcatagcagtgaggcatccccaatgtctttgcaccctgtaacctggcaaccatctaaagatggagatcgtttaattggtcgcattttattaaataagcgtctaaaagatggaagtgtacctcgagattcaggagcaatgcttggcttgaaggttgtaggaggaaagatgactgaatcaggtcggctttgtgcatttattactaaagtaaaaaaaggaagtttagctgatactgtaggacatcttagaccaggtgatgaagtattagaatggaatggaagactactgcaaggagccacatttgaggaagtgtacaacatcattctagaatccaaacctgaaccacaagtagaacttgtagtttcaaggcctattggagatataccgcgaatacctgatagcacacatgcacaactggagtccagttctagctcctttgaatctcaaaaaatggatcgtccttctatttctgttacctctcccatgagtcctggaatgttgagggatgtcccacagttcttatcaggacaactttcaagccaaagccttagtagaagaacaacgccttttgttcctagggttcagataaaactatggtttgacaaggttggtcaccaattaatagttacaattttgggagcaaaagatctcccttccagggaagatgggaggccaaggaatccttatgttaaaatttactttcttccagacagaagtgataaaaacaagagaagaactaaaacagtaaagaaaacattggaacccaaatggaaccaaacattcatttattctccagtccaccgaagagaatttcgggaacgaatgctagagattaccctttgggatcaagctcgtgttcgagaggaagaaagtgaattcttaggcgagattttaattgaattagaaacagcattattagatgatgagccacattggtacaaacttcagacgcatgatgtctcttcattgccacttccccacccttctccatatatgccacgaagacagctccatggagagagcccaacacggaggttgcaaaggtcaaagagaataagtgatagtgaagtctctgactatgactgtgatgatggaattggtgtagtatcagattatcgacatgatggtcgagatcttcaaagctcaacattatcagtgccagaacaagtaatgtcatcaaaccactgttcaccatcagggtctcctcatcgagtagatgttataggaaggactagatcatggtcacccagtgtccctcctccacaaagtcggaatgtggaacaggggcttcgagggacccgcactatgaccggacattataatacaattagccgaatggacagacatcgtgtcatggatgaccattattctccagatagagacagggattgtgaagcagcagatagacagccatatcacagatccagatcaacagaacaacggcctctccttgagcggaccaccacccgctccagatccactgaacgtcctgatacaaacctcatgaggtcgatgccttcattaatgactggaagatctgcccctccttcacctgccttatcgaggtctcatcctcgtactgggtctgtccagacaagcccatcaagtactccagtcgcaggacgaaggggccgacagcttccacagcttccaccaaagggaacgttggatagaaaagcaggaggtaaaaaactaaggagcactgtccaaagaagtacagaaacaggcctggccgtggaaatgaggaactggatgactcgacaggcaagccgagagtctacagatggtagcatgaacagctacagctcagaaggaaatctgattttccctggtgttcgcttggcctctgatagccagttcagtgatttcctggatggccttggccctgctcagctagtgggacgccagactctggcaacacctgcaatgggtgacattcaggtaggaatgatggacaaaaagggacagctggaggtagaaatcatccgggcccgtggccttgttgtaaaaccaggttccaagacactgccagcaccgtatgtaaaagtgtatctattagataacggagtctgcatagccaaaaagaaaacaaaagtggcaagaaaaacgctggaacccctttaccagcagctattatctttcgaagagagtccacaaggaaaagttttacagatcatcgtctggggagattatggccgcatggatcacaaatcttttatgggagtggcccagatacttttagatgaactagagctatccaatatggtgatcggatggttcaaacttttcccaccttcctccctagtagatccaaccttggcccctctgacaagaagagcttcccaatcatctctggaaagttcaactggaccttcttactctcgttcatagcagctgtaaaaaaattgttgtcacagcaaccagcgttacaaaaaaaaaaaaaaaaatcacaggttgcaaaccctggtaacactgcatgcttaatgttgtgtcttctgagcctgtttctagggatacaaagcaatcctgtgttctcagaggaagttgcacacattgtgccctaaagaaggccctcaggtgaaagagcagagctgtgaagaactatcagatttggaattcaatgacactcgagttctggtccaatctgaagccatggattaatctcaaagaatcagtcagtttcatgcaacagaagcccttttcaatggcacctttatatttttatcattcctttttcttcatttatctaaccccaaagccctgatatgccacagaaatggagctatacagccatgaagcggtgttacaggtgaggagtgtaatcctaggaagcatcaggtgaaaagcaggagaccaaagaagtggtcaggaacaatcatcagccctcctctgggcgggaatcagagcagtcagtccagcaggaagagtggcagactttgtagctccatgggcacgtcaattactaatgctaagatgtgttggactctgaaaaacaaaattctgtggctacactgtactgaatgaaattaaagaaactttttttgcatggacacagattagctgaatacttaaattattttcttggggctgcaacttgcaaaaaaaaaaaaaagaataaaaatcagccattttcaacaatttatattatttttaaaaataaatttcactagtgcatggttttaaaaaggagagagaatgcaacagggtgatacaaagatacaccatgtttattctttaatcatagtctgtgttttggcagacattacaaatggaaatactttctagaagatacttaaaattctctttatgtgacaaataagtataatatattcaatttatttccatgttaaatatacaaatcttatgaagttcaatatgtgcaaatttttcacatctttctccttctctcactttacctcttctccctcttttaaacttttctttctccctgccagagtgaaccttatactaaaaaattacaagttttgatctgatcctctctcataccccatgtttgattcagagctgtagatgcctctgaatttgcgaatttctcaagggaaaattaactttaagagctttctttatttcaagcatgttgaaaaggattttgcaacatgacttgggagtacattaaagtaagtcagcatgtatttgacgaagaagatatttgaacttttgcagtttattgtacagtgcatggtaattttttcacctttaaaattcagtttacaggaaaattctaaaatcatgttgccattgtgatgtccaataaatttgtttttagcaccagcattattcatacaggggttaaagtattatttgtagaaggtcttaggttttgtttgttttttaatcatttaaagcaatttctttagccagtttccatttactatgtgaatagaagcactgctaaaaattgggaaccctgaaacacagggctgtttattaattcatttttctgtagtaaaattcaatttttcacaaattatatttctaaagaaatatagtaaacataaatttgcaacaattttaaagctccagtttttaggtgactcaaagaaagtcattatgcctattaatagttatttgatgccatcaccaaaagtctatgtgaaaatctcctaaagtcaaaacccctgcctttggttttacagacggttattaccattgggtggagctgcaaggtcaaatttctcctaagttcccctatttagaggaaaagtcactggttattgtaataaaccacccatggttctttatgtacattttgataacacattattatagcttgattttaattttttgcattaatttttgaaatccacatacatctcatttgtttaaattaaggccatgcacaaatattttttttagttcagtgctgaccattaaaaactatcatgcttgatacggtgcaaaagttaaaatgagtatcactaaaaatgccttctttttatgtggtgcaatatgaaatacaccaagactgtgtcttgacattctgatggacccaggtaaagttgttaaaagaacgaataaaactttattaaaataatttagacacctgtgtaccagcaacaattgatttaatagacctatagtgtctatactatcccttagaataaaggtttatgattttcctgatactaagatgcagtcacataatcttttgtgcatattcctatacaaattatttctaattttaataagaaggacgtgactacggaatatttgtacatacttgtcattatgcagtatttatttaaaagttggtgtttttttttaattttcacatctgcacctcgacttgtggtttagtcatgtaactagcactatgccagtgaccgttgttgccctgtacatagtatgtttgaaaagtaaagggaattccagttgggaaaaaagggcagattagtcctgtaatgaacaccaactaatgtaaatcaaattcattctggtgatggtatttaacactttaaataaaacattttctttacaggcgtctgcagtgctttctctgacttttctccccacacagccctgagcctgctgcagctcattccctgaactcatgtgtcatttaaagaatgaaatcaccgtctcctacttctcgataacataagtggactgctggtcttagcagcggccctcagtagagcatttctttaaaacgccaaaggatttctgctcacactatgaaaaggtgctgttttttaaaaggttgttattttggattgagtttctttctgattaaatgactcagcaactcacagattttttgagtgaaatttttaatttagtcatggccttcactgacagcatagtcacaaatactcaggcacaggctctgctagcccctgggtgaagatggcgaaggcataactggctttatgcagcatatgtgtttctgctaaagtgtcagttttgctttgtggggagtggagggtgtgttttcgggatggggagacgtggtaacctgacatgtaacaacctgtccggagactagcttctacgtgtggatatgaatgggtgagaggatttctccatatccttctggggcgattcctcaactgggagaaggaaaccctgcagagttctcatgggagtctgcttcaggtttgaaatttaagagctagtttggattcatgtttagtaatcgaactgaaatctaagtctagctgtctctctattcttggaaacaaccatttcctccatttccaaagactcaactcgagtccaattccccctatctgtcccatatatttttcctttatcccatatataccccctactctagtgaattgtttctttgttgttcattcctgttctttgttgttcatatacattcctgtatatgaacaacattttcctttatcccatatataccccctactctagtgaattgtttctttgttgttcattcctgtttctttgttgttcatatacattcctgttataaaaattcccttccctttcttatgtgccctctcctgaaaagcccttctacttttctcaataatgatccatgcgagtcccttcttgcaactcccagctcacgaatgagctctttcggcaactcctgactaaaccctaacaacatggctgccattgatgccaacaccttcactttcccagggaccccagatgccaaggctccataggcaacaataaaggatatgatggtcctgtagtgggtatgatagaattaggcaagagatcaccaaagctgtctgcctactactgatgtaaaccttgacattctgtgcacgtaaaaatcatgtgctcaatgtgtttgtctcaactccctcagctcgtgatgccctcagggttctgtgggcatttatgcactgaagaaacaggagttcacacatccacctctggactgtgaaatgtgtattgagaaatactttgcaagagagaatttttttaagtgaacaaacaacaagtctgtgccacacacatcttccatatgccctgactcaggtcacttaatctccaggatttcatttcctcacctggaaaatatggagtttgaggtagattctcatctatcattaaatcaacactttaactaaaacgtaagctccttcagggcagagaccgtatcttcagtatcaaaaacaatgtttgacacatagctgctcaataaacatctgtccaatgaa

SEQ ID NO: 62 - DNA, 10087 bp, Homo sapiens
  source 1..10087: mol_type = other DNA
ccttcgccacgccgccgcgcagcccctccatcttcctgctcggcaccgggccccgcgcgcccctgcctacggggtcccgctgctctccggggctcctgccagccccaacccccggccccggtggcctccccccacccccgcccgggtccccctcctccgccacacgcgcgcgcgctcacacacacacacacacacacacacacacacacacacatatatacacgccagcgagctgctggccgctcaatggaccgatttccccggtttccctgaacccagcccagcccgggatgagaaactgcaaaatggcccgggtcgccagtgtgctggggctggtcatgctcagcgtcgccctgctgattttatcgctcatcagctacgtgtccctgaaaaaggagaacatcttcaccactcccaagtacgccagcccgggggcgccccgaatgtacatgttccacgcgggattccggtcacaatttgcgctgaagtttctagacccgtcattcgtgcccattacgaattctctcacccaggaactccaagagaaaccttctaagtggaaatttaatcggacagcgtttttacatcaaaggcaagaaattcttcagcatgtcgatgtaataaaaaatttttctttgaccaagaatagtgttcggattggacaactgatgcactatgattattccagccataaatatgttttctctattagcaataacttccggtcacttcttccagatgtgtcacccattatgaacaagcattataatatttgtgctgtggttggaaatagtgggatcctgacagggagccagtgtggacaagaaatagataaatcagattttgttttccgttgcaatttcgcccctacggaggctttccaaagagatgttggaagaaaaaccaatcttaccaccttcaaccccagcatcctggaaaaatattacaacaatctcttgactattcaggaccgtaacaactttttcctcagtttaaaaaagcttgacggggccattctttggatccctgcatttttcttccacacttcagcaactgtgaccaggacattagttgacttttttgttgaacacagaggtcagttaaaagtccaactggcttggccgggaaatataatgcaacatgtcaacaggtactggaaaaacaaacatttgtcacctaaacggctgagcacaggtattcttatgtacacccttgcatcagcaatatgtgaagagatccacttgtatggattttggccgtttggatttgaccccaacacaagggaagatcttccataccattactatgacaaaaaaggaaccaaatttaccaccaagtggcaggagtcccaccagctgcctgctgagtttcagctgctgtaccgaatgcatggggaagggctcaccaagctgactctgtcacactgtgcctaagaactccaaacggaaagcgccaaatggctgtttaaaaagtgccccaaatcaaattgaatagccttcagaatagaaccctagagaatgtcttataaggattgtctgccatttaaaaggaaagatgtcttttctcttttgcactgctcttttaagagttttagcagatttagcaggacagatgcattgaagccacatggtttagacttgattgataaagggaatgttgcatttgggactatgctgctaacgaaatggtttgaagtattttcatgtttggattttaataataaactgcctctcatttttatgaggactagagctatagtttctgcagctctgctcagatagtcctcataatcagaggcctctggccaactggggcaggacctgttttgctggtgggatcagactctgaaaaatggaaacgtaaaaaactggtttgcatatctcatcttctatctatctccctatctccatctctatcaccatctcactccttccctctgcaactactccctgccctaccaccgtggagtttaataatttgctaggaatcctattgaattcgctttgcttgtatatgttgcatttgtacttgatgtgtttaaggttctgggtaactttgatgcttcatgacaaaataggcaaatcacgatggactcccttgtgattccttttttaaagaaatgcctctgatgcagtcctcaccagtccattcagtcattctacagcaagatggtccctctgtggtgagtgggatataaggcagtgcatctttcatgatcacaaagaaagcctatgttgtggatagcattgcgtctcttgatgtaggcattgttatggcaaataatagcactgtggccacatctgtaaggccattccagcctaaatttagtgtctcccataatcccaacccatgcactaggctgggtgttctctcagaatatagaggcaatgaatcagcttaagaccctgggacacacaaaggaaaaggaagaagaatatttagaagttgttatggaatcctgtgtaccctaaagtgtggtaaccatgttacccattccagctgggttaaagtggaggaggatcttggccaggccaggtgcccaggtgactagtgctgaggtgcgatcgttttccgtatcattgtgtgcaccaccaccaatccagaaccatttgctctctttgagaacattgtagcactggatattccctctttccactgaagtcagatctatacaactacaaattaagcatttcaaaagaaaaagccattgtgcagtggcaagagttggaatccaagattatgtagacctgcgatgaaggaagtagctggccattctcattttctttatttcagccatatgagtagaaatcaaatcattcccatccctggctttttactgagcagcactttgttcttccaattcagcccaacgtctggccaagttgatgttaaattttaataagagtttctttcttctttccaaatgccagtcaagcatatttgttatgcatttgagtggggtagctcattcttttcctgaaatctcatctagattgcttgcaatgcgacaaataactgtttaaaaaatgtgctgtagttcagtgatgagctcattgcccctctttaggaaggtaagatcatgcctaaaattaaaccgaatatggtaaggaaagacactatcgttatgctcacgttctgcctggtcctaaacctgccttattattattttcagaaagcaatctaattagagcaacttgcatagatctagatattaactaatgaaagaaacaccaagttcaaatgtgttaccaattatgacacacattacaatataattataaggggagacattaaaaacattttgtaattcagtggtataatttgatgggtatcacaaccacaccgtgtttgttccctgtccctttgaagaggactgtactaccatcctcacactgtgctctgttgggatcactgagagattgacaactgacagaagtgtttagttggctgttcctgagcacttagttcatttggttatagctaagtgatattaatgccaatattacttgggccagattctgactctggagttttaatcccaatgtgcccattcattttcactaaatataaatgcatagacactttaaaccaagaagattaaaagaggaaaagaccagaatctctaaaaaacagtttactgttctgtagtggacaatttaaattttatctcatcataatgccaaggatttttgtttgttttttaagttcagggagattttttaaatctccttgcagtcttggctccatcttaaccttagagtgggaggactttcccatcccccaccaccgtgttatctatcactttaatgaaaactcaaaatagtgaggaaatggacttttcttcagaacttgaggggtttagagatcaggctcatctctgcttataaggaggacttcctggagatgatcccaatagatcacccttcacttttgaagaatagcagcttctagctgaccatgcaaaatctctgtttgtttttaaagactgtttcaaagattaaatctatacaaccacttttatgaccaaaaaagttgttgtgtcccaagtgtcctaattagaaaggcagctggctctcctgcagctcccaggcttgtgtagtcaagtgcagtgaactgggcagtaatcagctcaggaccattggctctttcctcctctttcagctacagacacatgccctggcttttgcattgaccttgctttgttagttctagactaatttatccttttgtcagacactttcccttttttaggatactcacaacagagtccactttcagctattggaatacattttaccataaagcccccaaggctttctacccataacaccaacacaaaaaaccaattaagttgcagaggggggaaataatcacaaacttttggcaacctgcttaatgctagacgctctgttttcactggttattctgagtgtcatatgcagatgcagatccaagggagacagggctaaatggtggtgtcaagaattcctcccacaccccaaaaattctttccaatatttgattattaaaatatctatcatttctccaccttgtgtctgtgtcttaagtgtctgtgaatattgtaaaagtgctgtatgtttagtagtgttgtgtgcctggcagtgctgactatgactactgtgccatctgtctgtgaccttgatgtcaggtacctggccatggggctaccagcaaggatgtgcaaaggaagaaccgctgcccctgccctcagcttccttatgcccgagccactacttatccgtgaatgtgagtgccaagagaaacctaatttggtggggaagccaaggaatgggagaacgttttttctgatttgagtcaaggcactaattattagacactctcagacaacaaagcggtattgacctgagatcaaaggaagcagggacaatattgtagaatgctaggacactggaaatgaagaggccttcagcttcgccagtcttgattcttgactttacaaatgagaaaatggagaaacagggaggctaggtgatttgccccagatgacactgtcaattagtggagaaattatgattcctcctctagtacttcttggtcttaccagcatcaaatgaagctgggctcacagggaaagtggtttttggtctgggatctgcagagcacataaaataagttagaaagtctataaaagaggaaaaaaggttctcctctccctcccaaaaatgacttcccaacacagaacaataaaggagatgcttgtgtgggtatgcaactgtccaccaattatcacctaccttctccctgagatgaaacaaaacacgcttggtaaatcatgatttgataaataacaattaagttactggtggttcatgcttgacagctgcaatcttacccaataagctattcatcttagtaaaaccttgttttagtaatatacatgcataatttagaagttgaaggctgaaaattgttgtgttcttctccatatatatacaactttcatcggggtcccttgagttaagaaatgccttctattttataggtaatctcaacagcagactttgaaatattgtgtttggttcaatatgctaagaagatatacaaataataataacttcgtgagtcttcatgtgcaaaatgacaaactgagcaattttctgtataattctagattcttaaaaccttttttcccataattgtttgaatgcactaggcacaagtttctgtttaagcctctgcttctccctgaatgtgtagaactgtcactttgcttacaaaggggctgtgtgtttgtggctggcagcccagatagagcactctgggtgctggggattgagggcaccttggccagctgattgacctgtctggactctgtggcccaattaatcgaggccacagatcagttggtactaagcatcaaaatgattcattatgcctctctcacactccgattgctttgccacatagataacagcatttgaaatgggaaacactttagaatgtcattattgtattatatattgtcatcttatgcagtatttctgaaaatttatttttattgtgatgactgattgaagacatatgtgtatgaatagatgttcctcagtgaagcctattctcttgttgttgatgacagtggcatcagagtgaagagttgttgcagagtaactgacttcagtttaaggttaccctggggttctggggttaggacccggattatgcagctgatctcccagcaaacacagctgtatgaaataagcaactcttcaaaatacaattcttgccactcattctggacctttgattcataatgtcctgggataagtccaacttacaaagattcccaggtgattcgtgtgaccaccatttggaaaccagtaatatatacatccggagtcatatataccattttgaacaaagtagctaaatgcttatctctccttgtgcgattagtttcacatgttcgttgaggttatcatgttaaatggcaatatagacacagtcttagtgattcaacaattcagctgtttcagccgaattgaccagttgctaatttataagcaaattgtaactgatcaatttagtcagaatggatgcaagcccaaagacaaggtggagagcaagctttaatatacactttgtctcaaggccagctcttcatatttccctctttatatttcaaactaagaggcagatctcaggtcactcatctttagaattggtgagaatgagaaacagggaactgaagtgattcattcagattcaatttggaagtcattcccagagcctaaaatatttatctaaggggtctgatttatcaacctcaggtagtctccaaaacacaaaggtattcaggctaaaataaaaccaatcaaaaaaggaaattcaggaggctccagttctaagactaacccagtataacaagggtgatcagttattacttgtggccttgtaccacctctgttggataatgtgccaccgtcctaatgaagctttggccagacttctttctatagctgtgcacatagaaaggctttagacaacagtgataccaaagggttcctttgattttccttctgtgtgattctcagacagttttagctctttggtgtgctgccaaatgtttaacaactggctctggaaaacagtggaacccagatttttagcacctaccaatttctatggtgtaaatactctcatctcagccaatttcaggctactaacttaatgtcaaccagctgaaaacacaacaatatgttttcaagagccagtataagttggctccaatacagcatgagccagctccagcatagcactgcctgaacaatttttaagtcagtgagtaccaaataggagaggctagcagaggctttggagggacttttaaatgtcccctccaccaagggaagcatttccatgttggttagttttactgatttttagtaaaagagcatattattggatcccaaggtgttcaaatattccacgtgactcttaggaacctctcaacagactccccagtatcttgggagattaccaactgtggcttttctaatttctaagggtgaccctccaggaaaacattctttaatttaactaccagttaaaaccatgtttggctgatcttttttaaaaaatctatacctatagttcatcacttgtttccccaactttgtcattttccctatccacttgcccctcatccttctgacacaataaaaaaaatacaggtacccacaaaacagtgcctgcaaaggagcaggggaagaacaaggcacccagtaagctcacttggctctacatatctggaaaggcactgaggttccaaggtctctaccccaccaggggacacccagtaaacatcacgctgagagtttaagttgcactcaactggagttgatttgcccttagaaaagtttccaacggtgaagaaataccacttggtaggaatatctctgggcaacttcattgctgactgggcaaactttgtaggataaggtgtcatgtgatgtaaggctggaccatgaaaaacaaaaatctactaatacacatttggcccctgcagtcccctggcattgagcaatagagcaactgtcctttctcaccactagttgtgagtgtacttccaactgagtgtttagaaatcaggtaatctggtaatccacagaacaagctttgtttgcaaattgcaaatttttctggtagaagtcattcttaggtgggcttcattaaatctttggagaggcaatgctgggaaaaaatatgcctaattcctaggacacagtgagtggccgtaagttgttatctgcatgtactaaatcaaaattagatcatgaaggaccaaatctacttccgttgctataataaaataccctacaggttctgtaaggcatttctaaactgtaaatgcttgaaacgcaagttggacattttctatactatgtgtaaaatgccaggttatacctctattttctttcttcctaatggtcattgaaatgagcttgtttctctacattgagcaagctacattttattttaaatgagtcaggtgcatgttcagatgtctgtattcccaatacattgcatgcagcctgagcacaagtatgccttcaccctctggctcttttcctgacaccaaacagagaagtccacagctacaagcacaaggggcttgacaggtaggccttgttttattatgaacaaattcaccagaaaaccattcttgaggaaccagccactcccatagcatttttagtcttagggaagaaaattggcagggaagaaaaattggcagacaatgggaaagaggtttggtcaacctgcataagtggaataaagtcattgaagtacttgcaaaaagaagagggaggtttccctctgaaggaatctagaccattttttaatatggagaataactgtaccccatgttagaagtgctctttagccatatttcaagatccagatctgaacccatactgtgcagaaaagcacaagagcgcttacagccaagagccctgggaggagttgagtaacaggctgagatccggtcaccaagcatcattagttagcactgaaattctcccaggacacaaaacaaatgtatggaaacatcgcaatacactatcatttactcacatgggacaaccacctaagtccatagcaaaactttacagatctagatgttatctcaagtcaggattccagatcaacatcctccatgtggagatgagacaactgaggttcagaaagttggagtgacttatttgcccagctagctaactggaattagagtctgggtctcccaaccactgattctgctacaccacactgtgctgcttccattagttagatggacattgacaattaattgtgaaagcattaattagaagtcaagacaaaagaatgtttgtgatatcttctctcgcttaacatactttgatttcaccattgcatttcagcattttaaagaatagtgtatcagtatagaaggaggagcaaagctctttaagagtaatgatgaaaccaaaaggtcaaaaagtagaagaatagcgaatgtaagttattaaataaatgagcaagaagtatgtgtcagggaaaataaatgcttcttatattgttaataccgtgattccctgcccgctcaccccgtcactcaacacttgatgcatttgttttggaaagtgccattttatgctgagatactggtattgaaaacttccctctttcccaactctgtcagaagttctcttgctctttggaagaagccgagggccttaacaaatcagactgacctcccttgtgacaggcagcctcgcctgcccttggaaacaagctgccagaatgtgagggccacagagacccaagagaagccagcactgctagactgaggaacttgtaaatatcttctgctttcttggtgaacaaggacagaggcaattgcgtgtatattgtgactgtagtttgtgaagaaaatgcaaccatttgcttcgacagctcctcaaatgtacttgttaagtgtgaatgtgcctgcctcattgccttgtgttccaaacacagtactgaatgcgttgtttttaaataaaccatttcgttttgctttgggaaa

SEQ ID NO: 63 - DNA, 841 bp, Homo sapiens
  source 1..841: mol_type = other DNA
accggtgagtcgccggcgctgcagagggaggcggcactggtctcgacgtggggcggccagcgatgaagccgcccagttcaatacaaacaagtgagtttgactcatcagatgaagagcctattgaagatgaacagactccaattcatatatcatggctatctttgtcacgagtgaattgttctcagtttctcggtttatgtgctcttccaggttgtaaatttaaagatgttagaagaaatgtccaaaaagatacagaagaactaaagagctgtggtatacaagacatatttgttttctgcaccagaggggaactgtcaaaatatagagtcccaaaccttctggatctctaccagcaatgtggaattatcacccatcatcatccaatcgcagatggagggactcctgacatagccagctgctgtgaaataatggaagagcttacaacctgccttaaaaattaccgaaaaaccttaatacactgctatggaggacttgggagatcttgtcttgtagctgcttgtctcctactatacctgtctgacacaatatcaccagagcaagccatagacagcctgcgagacctaagaggatccggggcaatacagaccatcaagcaatacaattatcttcatgagtttcgggacaaattagctgcacatctatcatcaagagattcacaatcaagatctgtatcaagataaaggaattcaaatagcatatatatgaccatgtctgaaatgtcagttctctagcataatttgtattgaaatgaaaccaccagtgttatcaacttgaatgtaaatgtacatgtgcagatattcctaaagttttattgacaaaa

SEQ ID NO: 64 - DNA, 1330 bp, Homo sapiens
  source 1..1330: mol_type = other DNA
agactcctgggtccggtcaaccgtcaaaatgtccaaagaacctctcattctctggctgatgattgagttttggtggctttacctgacaccagtcacttcagagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagccccaccaccacccgacaaatgacaacaaccccagctgcacttccaacaacagtcgtgaccacacccgatctcacaaccggaacaccactccagatgacaaccattgccgtcttcacaacagcaaacacgtgcctttcactaaccccaagcacccttccggaggaagccacaggtcttctgactcccgagccttctaaggaagggcccatcctcactgcagaatcagaaactgtcctccccagtgattcctggagtagtgttgagtctacttctgctgacactgtcctgctgacatccaaagagtccaaagtttgggatctcccatcaacatcccacgtgtcaatgtggaaaacgagtgattctgtgtcttctcctcagcctggagcatctgatacagcagttcctgagcagaacaaaacaacaaaaacaggacagatggatggaatacccatgtcaatgaagaatgaaatgcccatctcccaactactgatgatcatcgccccctccttgggatttgtgctcttcgcattgtttgtggcgtttctcctgagagggaaactcatggaaacctattgttcgcagaaacacacaaggctagactacattggagatagtaaaaatgtcctcaatgacgtgcagcatggaagggaagacgaagacggcctttttaccctctaacaacgcagtagcatgttagattgaggatgggggcatgacactccagtgtcaaaataagtcttagtagatttccttgtttcataaaaaagactcacttattccatggatgtcattgatccaggcttgctttagtttcatgaatgaagggtactttagagaccacaa

SEQ ID NO: 65 - DNA, 2728 bp, Homo sapiens
  source 1..2728: mol_type = other DNA
ctttcctacgcagccgctcctgccgccgtggtcgctggagctttgcctctctaggccggcagcgcctctcctccatggtcctgtctgtcagcgctgttttgggagcccgccggtgaggccgggccacgctcagacacttcgatcgtcgagtctgtcactgggcatggcgggtcagttccgcagctacgtgtgggacccgctgctgatcctgtcgcagatcgtcctcatgcagaccgtgtattacggctcgctgggcctgtggctggcgctggtggacgggctagtgcgaagcagcccctcgctggaccagatgttcgacgccgagatcctgggcttttccacccctccaggccggctctccatgatgtccttcatcctcaacgccctcacctgtgccctgggcttgctgtacttcatccggcgaggaaagcagtgtctggatttcactgtcactgtccatttctttcacctcctgggctgctggttctacagctcccgtttcccctcggcgctgacctggtggctggtccaagccgtgtgcattgcactcatggctgtcatcggggagtacctgtgcatgcggacggagctcaaggagatacccctcaactcagcccctaaatccaatgtctagaatcaggccctttggacatcctgctgacacttgggccccttaacaccttgggctgctcagaccctccagatgaggtccagcccagatctgagaggaaccctggaaatgtgaagtctctgttggtttgggagagatagtgagggcctgtcaaagaaggcaggtagcagtcagcatgacagctgcaagaatgacctctgtctgttgaagccttggtatctgagaggtcaggaaggggacctctttgagggtaataacagaattggaaccatgccactcttgagccacaatacctgtcaccagcctgttgttttaagagagaaaaaaaatcaaggatatctgattggagcaaaccacttctttagtcatctgtcttacccccctgggacagctgttacctttgcagtgttgccgaatcacagcagttacctttgcagtgttgccgaatcacagcagttctgttggagaaacgcttggtttccggatccagagccacagaaagaaatgtaggtgtgaagtattaggctgctgtcagggagaggatggcagatggaggcatcaagcacaaggaaaatgcacaacctgtgccctgttatacacacgttcatgtgcacccaagaacctatgactttcttccagttccttctaccaggtccccatcctgctgccagctctcaacatagcaggccataggacccagagaagaatcccagcgttgctcaaagtctaaccatcataaagacactgcctgtcttctaggaatgaccaggcacccagctcccactggactccaattttttttcctgccttatttagaattctttggcgggaagggtatgatgggttcccagagacaagaagcccaaccttctggcctgggctgtgctgatagtgctgagggagataggaatttgctgctaagatttttctttggggtggagtttcctctgtgaggggcttgcagctatccttcctgtgtatacaaatacagtattttccatggttctgcctgcacttactttgtaatgccacggttgagattgagagagatcagcgcagccaggcaagggaactttaaagaattattaggccaccttctccctttcctggaccccagagtcattcctccatttggttaaaatactcagtgcagggaactcttacatcctgtctccttcacttgcagcgtcccctgctatgcctcaggtgaaccacataattcttgggtttccgttcctacttgctagtgatttctgaacatgttcaatggagcggcacacagtctagacccacttccgcattgaaaccttcactgttcctctttggtttcttcagagctttcccaagagagctgtcagttttcagctgtcagtaacacaaatgagtttatggtaacacaaatgagttttgctatctctctgagaagctcatctgacctcctgactctcagccctacagagtagggagttgatgctgacaggatgaagatttaggaataaatatgcctgggaagagactgggaaggttctagggtgaggcacctcagtaactcatggtaccttggccaagttggaaggaagcagtttgttaatgaggcacagtaatcctggctgcagggtctaggaggtaagaccagctgggatgaccttccctgggttaatcaatttccctctagacaacacaaactgcaggcatgtgactaactttgaaagaacacccatcatgtggctgctgtcacccttgaccagccgtggtggtggttactccatctgtggttggagcgcctctttgggattcacttcaaggtcttgtgcctatttttctgcatatcttctgtgatgacaaatctctgtcccctgagtgttaatttgatttttagaaatggccaaaagtcacgtgatccaaactttttttcagtaatatggagactgagctgcatggtagttggggatcaaaaatatgtgaccttaatgagatttttatgatttctaaagtaacaataaaagcagtttttagagttgagttccagagagggcagggcaatggcagtgacatgtttgtcattttaataataaataacatctattgagtgcttaa

SEQ ID NO: 66 - DNA, 894 bp, Homo sapiens
  source 1..894: mol_type = other DNA
gtctctggtttctggccccttgtctgcagagatggctcccaatgcttcctgcctctgtgtgcatgtccgttccgaggaatgggatttaatgacctttgatgccaacccatatgacagcgtgaaaaaaatcaaagaacatgtccggtctaagaccaaggttcctgtgcaggaccaggttcttttgctgggctccaagatcttaaagccacggagaagcctctcatcttatggcattgacaaagagaagaccatccaccttaccctgaaagtggtgaagcccagtgatgaggagctgcccttgtttcttgtggagtcaggtgatgaggcaaagaggcacctcctccaggtgcgaaggtccagctcagtggcacaagtgaaagcaatgatcgagactaagacgggtataatccctgagacccagattgtgacttgcaatggaaagagactggaagatgggaagatgatggcagattacggcatcagaaagggcaacttactcttcctggcatgttattgtattggagggtgaccaccctgggcatggggtgttggcaggggtcaaaaagcttatttcttttaatctcttactcaacgaacacatcttctgatgatttcccaaaattaatgagaatgagatgagtagagtaagatttgggtgggatgggtaggatgaagtatattgcccaactctatgtttctttgattctaacacaattaattaagtgacatgatttttactaatgtattactgagactagtaaataaatttttaaggcaaaatagagcattcaaagccagcttggaatttaattctgtcttgataccttgttatttatgcaaaaactcctatctcctttcctttatgacaagagagtaagttttaggttgggatcc

SEQ ID NO: 67 - DNA, 5087 bp, Homo sapiens
  source 1..5087: mol_type = other DNA
agttttgctccgaaagacttaccgaggagggagcttgcggtgcgttctgggaaagttgctgggccagctcctttgtttccagtctgagcgttgcgttcggtttcccgagggtcttctgaggcaccgcggctgcgggcttctgagttcccggctctccgcagggaagcctcctcttcgtacctcgttttttggctcgtggggggtcctcccaccgctggccgacgcagccagcatgtccggggtgcgcgcagtgcggatcagcatcgaatcggcctgcgagaagcaggtccatgaggtgggcctggatggcaccgagacgtacctgcccccgctgtccatgtcgcagaatctggcgcgtctggcccagcggatagacttcagccagggttcgggctccgaggaggaggaggcggcggggaccgagggcgacgcgcaggagtggccgggcgccgggtccagcgcagaccaggacgacgaggaaggagtggtaaaatttcagccttccctttggccttgggactcagtgaggaacaatttgagaagtgccctgacagagatgtgtgttctctatgatgttctcagtattgttagggataaaaaatttatgactcttgatcctgtctctcaggatgcacttcctccaaaacagaatcctcagacgttgcaattgatatctaaaaagaagtcacttgctggagcagcacaaatcttattgaagggggcagaaagactgactaaatcagttaccgaaaaccaagaaaacaagctacaaagagacttcaattctgagcttttgcgattacggcaacactggaaacttcgaaaagttggagataaaattctcggagatctgagctacagaagtgcaggatctctctttcctcatcatggtacatttgaagtaataaagaatacagatctcgatctggataaaaagatacctgaagattactgtcctcttgatgtccaaattcctagtgatttagaggggtctgcatatatcaaggtttcaatacaaaaacaggctccagatataggtgacctcggcacagttaacctcttcaaacgacctttgcccaaatccaaaccaggttccccacattggcagacaaaattagaagcggcacagaatgttctcttatgtaaagaaatttttgcacagctctctcgggaagctgttcaaattaaatcacaagtccctcacattgtggtgaaaaaccagattatctctcagccctttccgagcttgcagttatctatttctttgtgccattcctcaaatgataagaaatcccaaaaatttgctactgagaagcaatgtccggaggaccacctttatgtcctagagcataatttgcatctactgattagagagtttcataaacagaccttgagttccatcatgatgcctcatccagcaagtgcaccttttggccacaagagaatgagactttcgggtcctcaagcttttgataaaaatgaaattaattcattacagtccagtgaagggcttctggaaaaaataattaaacaagcaaagcatatttttctaaggagtagagctgctgcaaccattgacagcttagcaagccgaattgaggatcctcagatacaggctcattggtcaaatatcaatgatgtttatgaatctagtgtgaaagttttaatcacatcacaaggctatgaacaaatatgcaagtccattcaactgcaattgaatattggagttgagcagattcgagttgtacatagagatggaagagtaattacactgtcttatcaggagcaggagctacaggattttcttctgtctcagatgtcacagcaccaggtacatgcagttcagcaactcgccaaggttatgggctggcaagtactgagcttcagtaatcatgtgggacttggacctatagagagcattggtaatgcatctgccatcacggtggcctccccaagtggtgactatgctatttcagttcgtaatggacctgaaagtggcagcaagattatggttcagtttcctcgtaaccaatgtaaagaccttccaaaaagtgatgttttacaagataacaaatggagtcatcttcgtgggccattcaaagaagttcagtggaataaaatggaaggtcgaaattttgtttataaaatggagctgcttatgtctgcacttagcccttgtctactatgattttttccagatgtttcctaaagaagtttccagaaactttgacttgaaatgtttgcagatcaactataagcacaaagaagagataacttccaaaagagtgctgtttttaaaaataataattaggaaatgtttatttagcactttcaaacttttcactttataaatgacaagtgctttgaaatgcagaagtttatgtacagttgtatatacagtatgacaagatgtaaaataatatgtttttcatgcagtttaaaatattactaacttaagggtttctatgtgctttttaaaatattccttctttgatgttgacatcaaataaagtatgtggtttaaaaaaatctccaaatacctttttttccccccaaatactttctaaactttttttttttgagatggtatctcactctgtagcccagtctggagtgcagtggtgtgatcatggttcactgcagtcttgacctcccaggcttaggtgatccttctgtctcagccttccgagtagctgggaccacaggcatgcacaaccacgcctggctaatttttgtattttttataaagacagggtttttccatgttgcccaggctggtttcgaactcggctcaagtgatctacctgcctctgcctcccaaagtgctaggattacaggcgtgagccaccatgcccagcctactctaaattattgataacctcttcctccagttgtctcctttaagctttcctgggtctaacctacataggtaatttaagaacatcctcagaaaggacagctgaaggcaataggaggcagattatctctttagggcgtcctcaagtttttttggtctgttctcccacttgattgacctcaccagttgagacacctagtgtatggctcatgcccagccttccacctgggattctccagcctccacccagcagccctggattgctttctccaattaaggcctttccatcagctctctgctttttcaaagcgaaaaaactaatggattagtgggttatcttttccaaggaacaggtttgcacttcttggaaaaagtgcctaaagtgtgcccattaatatgaggatagatttaggctcataagccttttggtaacactgaaagtagtatcatataggcaagctctccttataagtaaggctttcaatttttaaaacagacatcctgctttaacaatttgtaagatgactgtgcagtaataaaagtcctttgtatttctccaccgtgttttcattaaagaaaaatggagcttgtgggccacgatagaacaactttgtgcttttttccccttctgatcaagatcttgcatctttctatccatggaaattaaaataattggtatgaatttgcagttatttaaaaatcttgagtgcttcaaaaattattgttgcctgcaaaatttgccttggtcaataggctaatctgcacaattccactcacataaggagtcttttatgtgattttgaaggctcaggctagaagagtgagtctgagacttttgctgaatgaccagtttttgtttatataaacttctcccattgcagattgatactttggtaaactaataaaaatgaattcctaaaatgaaattttgaaaagatacaaaataaaagccccatttatttgattataacttgattaaattgcatcaaatactagaatttatagacagagtctcactctgttccccaggctggagtgcagcagcactgttttggctcactgcaacctctgcctcctgggttcaaatgactctcatgcctcagcctcccgagtaactgggattacaggtgtgtgccaccacgaccgtctaatttttgtaattttgataagagggttttgccatcttggccaggctggtcttgaactcctggcctcaattgatccgcccacctcggcctcccaaagtgctaggattacaggcataagccacagtgcccagcccccccaaatataaacatttctgaatgctttattttttatttctctgcttgtcatgaatcagtaacaaatcatggaccaggaccacaccttgagtagaatggctgagaatacatgtgcagatactaccgtctgttcttttaaaccccatctgagtagagtgggataactgaagactttacgttcttcatgtcttactttccctgtttggtacgtcgctgtagtgagtagccagtaccgacctaaagaattgtagaaactaaagcaaatgtgtgggaaaatggtagcttagttgctgtggtagcaattcttatgccttgtatttatttacattttctagtttaatgttttaacctgaatttcctggagtttgaaggatgtgctatggaaacttgggagacagtttgaagaaaaccaattagcccctcaacaagtattaacaggttggcaaggagctgtgtttgaatcttggctctgctactggcttgctgtatgaacttggcaaggattctctgtgaacttgtttccttatatataaatgaagatagaggtgcctcctctactaacctcagtggtgattgagaagtttcagtaacgttggtaataaatgttaaattctaaagtactacataaatataaagcattaagcaagtgtgcttctaagagtcaagccaattagaaaaaatggttgagacaccagctgtatttattaggagaaagcatttcagaatgtcctgtattcatatttgtatgatgttttatatatggtgaagatattgagtgtttttcatcagatttctttgctggaacaccatcaaatcaaagggataacctgattatctcatgttgatcaggaattgtaattggcccttaaatgctgggattacaggtatgagccaccatgcctggcctccttaggtattgctgatgaataaaaacaggggcaactaca

SEQ ID NO: 68 - DNA, 4472 bp, Homo sapiens
  source 1..4472: mol_type = other DNA
agtcaggggtaggagcgggagccgagaggaggcggaggagatggcgtcccagccgccacctccccccaaaccctgggagacccgccgaattccgggagccggaccgggaccaggaccgggccccactttccaatctgctgatttgggtcctactttaatgacaagacctggacaaccagcacttaccagagtgcccccacctattcttccaaggccatcacagcagacaggaagtagcagtgtgaacacttttagacctgcttacagttcattttcttctggatatggtgcctatggaaattcattttatggaggctatagtccttatagttatggatataatgggctgggctacaaccgcctccgtgtagatgatcttccacccagtagatttgttcagcaagctgaagaaagcagcaggggtgcatttcagtccattgaaagtattgtgcatgcatttgcctctgtcagtatgatgatggatgctaccttttcagctgtctataacagtttcagggctgtattggatgtagcaaatcacttttcccgattgaaaatacactttacaaaagtgttttcagcttttgcattggttaggactatacggtatctttacagacggctacagcggatgttaggtttaagaagaggctctgagaatgaagacctctgggcagagagtgaaggaactgtggcatgccttggtgctgaggaccgagcagctacctcagcaaaatcttggccaatattcttgttctttgctgttatccttggtggtccttacctcatttggaaactattgtctactcacagtgatgaagtaacagacagcatcaactgggcaagtggtgaggatgaccatgtagttgccagagcagaatatgattttgctgccgtatctgaagaagaaatttctttccgggctggtgatatgctgaacttagctctcaaagaacaacaacccaaagtgcgtggttggcttctggctagccttgatggccaaacaacaggacttatacctgcgaattatgtcaaaattcttggcaaaagaaaaggtaggaaaacggtggaatcaagtaaagtttccaagcagcaacaatcttttaccaacccaacactaactaaaggagccacggttgctgattctttggatgaacaggaagctgcctttgaatctgtttttgttgaaactaataaggttccagttgcacctgattccattgggaaagatggagaaaagcaagatctttgatatctttcatgtttgcctgcagttgaacaatactttagagtactttttaaaattatttctcacaaagaaatgaatgtacaatccaatgaaaacatttgttattggctatttcaggtgttttgctgctagaaattattaaagttacacactagtatgttggtctggtgacctggttacattttattatacacattattggaccataaggacatttgtttcacctagattttaagattatggagactgctgtcatttttatcttatttaaatctctaggtttattggaagagtaagattgatgaactatagcatgcacagtttggtacagtagagatcattaatacttttaaaagttctgcattaattgacttggaatccttagaaatggagtggtgacatgtcagtatgagaacaggcaaaaggtaaatttttttttttttttacaaccttaagtaatctcaataataaaattttctgatctgtattatatccagtgttgggtttatattttcacccacaaacaactgacactgctatcttttactgtatttttaaaaatttaatttgaaaaggtcacccagaagcattctgaaggaaatggttctagaattatagagtatgtagccctaatagttttccctcctagcaaaaagtctgaaatttatctttcttacaaactgttccatttcttctaaggatcccttaattatttatctctttaagccaggcatggtggctcacgcctgtaatcccagcactttgggaggctgaggcgggcagatcacctgaggttgggagttcaagaccaccctgaccaacatggagaaaccccatctttacgaaaaatacaaaattagctaggtgtggtggcacatgcctgtaatcccagctactcgggaggctgaggcaggagaattgcttgaaccccaggaggcggaggttacgctgagccggagatctcgccattgcacttcagcctgggcaacaaaagcgaaactccatctcaaaaaataaaattatttatttctttaaaatatgactagttttcatactgggtgaatcagaagtatatgaaaggtatactttcttttcctacaacaaatacttgagttcttttgagtttgcacttaatgatataatcagttataatagtaatttttaatattttcatagattgtttgcttctaccttgtgtaattttttaaattccatatttaggatgctctgtaaatattgaaaatgtgtcacattggatacatttttcttttaggtttagtttttactactgagacttatttatagtcttagtgctctattgccatttagaatatgataatcctcatgcctttaatctcagcactttgggaggccaaggcgggcggatcacctgcggtcaggagtttgagaccagcctggccaacacggtgaaaccctgtctctactaaaaatacgaaaattagctgggtgcagtggcgtgcacctgtagtcccagctactcaggaggctgaggcaggagaatcacttgaacccgggaggcagaggttgcagggagctgagatcatgccactgcactccagcctaggggacagagcaagactctgtctcaaaacaaacaaacaaaaaataataatacgataatgctatttgacgtgttttttggtttataatgattttaaatgcagttaactttcagtacactgaatatttccccagaaaattggaaacttcatatacttggctacgaacatactacagagtaatactatcaggaatacaggtgtataagaatacatttatagatattgttgaaaactttgaactgtttgataaaaattgtgatttagtattttttcttttgtctttttttgaaacggagtctcactctgtcgcccaggctggagtccagtggcgcgatctcggctcactgcaagctccgcctcccgggttcacgccattctcctgcctcagcctcccgagtagctgggactacaggcgcctgccaccaggcccggctaattttttttgtatttttagtagagacggggtttcaccgtgttacccaggatggtctccatctcctgacctcgtgatgagcccgcttcggtctcccaaagtgttgggattacaggcgtgagccaccgcacccagcgatttagtatttttttctaatagactatgttcaacaaataagtaattctcgaatagttcagattaaaacatacaggaaccaagtacatacccagcatagaagaactttactaaaggcttcttggaaagcccttttttgaaacgacagtatcgtaagtaacatatcatttataatagaaatcttgacccagtgcagaaaaataaatatagtaaaatttatttatctttggccagttttccaacacccaggtattagccttgaagttgaagagataaggtttcttgtatattatttttcatttgtgttctacaattaaaggttctgctttgatttgtcagataatttatagaaattttgttctcaaaacaaatgtttgataaaacagattattaaatttggggttgagatgtctaaattgaatgctagaagtaaagtagaagtgaccactattaaagatgtatagggagaagagtacagcacaaagtgataaaatagtgacacttttgtaggggtgtttattgtttggttaagtctgctaaattacggtatgcattatctggtgactatttgtgcctgaaaattcgttttgtattaaaattctggagaaggaattcaggactagaataaagagaaattttgtaaccttttttatcatgacagttttagaataatttttttagctgagattaaatgttcaaggctccaatattatttttaggaacttatttaaggagtgctactttacagaaattactaacacaccaaaacattattaattaaataaaatataagtttacaataataaaacatgtttcttttaatttttctgattatattttatgagttcagaaaggaaatggtaaaagaactatacattttcatgttttaacattttatgtacgtacttgattctgtctgtgtcataattacacatttacttgaacacagctatcctttatcttgtgctttctttaatagaaaaatgaacagaaactgaatgcagttaaatttttatttttagtaggttgtgaagttacttttactggagaaataaaaatatgttaaacttga

SEQ ID NO: 69 - DNA, 1593 bp, Homo sapiens
  source 1..1593: mol_type = other DNA
atggaggaggactcactctacttgggtggtgagtggcagttcaaccacttttcaaaactcacatcttctcggctcgatgcagcttttgctgaaatccagcggacttctctccctgagaagtcaccactctcatgtgagacccgtgtcgacctctgtgatgatttggttcctgaggcaagacagcttgctcccagggagaagcttcctctgagtagcaggagacctgctgcggtgggggctgggctccagaatatgggaaatacctgctacgtgaacgcttccttgcagtgcctgacatacacaccgccccttgccaactacatgctgtcccgggagcactctcaaacgtgtcatcgtcacaagggctgcatgctctgtactatgcaagctcacatcacacgggccctccacaatcctggccacgtcatccagccctcacaggcattggctgctggcttccatagaggcaagcaggaagatgcccatgaatttctcatgttcactgtggatgccatgaaaaaggcatgccttcccgggcacaagcaggtagatcatccctctaaggacaccaccctcatccaccaaatatttggaggctactggagatctcaaatcaagtgtctccactgccacggcatttcagacacttttgacccttacctggacatcgccctggatatccaggcagctcagagtgtccagcaagctttggaacagttggtgaagcccgaagaactcaatggagagaatgcctatcattgtggtgtttgtctccagagggcgccggcctccaagacgttaactttacacacctctgccaaggtcctcatccttgtattgaagagattctccgatgtgacaggcaacaagattgccaagaatgtgcaatatcctgagtgccttgacatgcagccatacatgtctcagcagaacacaggacctcttgtctatgtcctctatgctgtgctggtccacgctgggtggagttgtcacaacggacattacttctcttatgtcaaagctcaagaaggccaatggtataaaatggatgatgccgaggtcaccgccgctagcatcacttctgtcctgagtcaacaggcctacgtcctcttttacatccagaagagtgaatgggaaagacacagtgagagtgtgtcaagaggcagggaaccaagagcccttggcgcagaagacacagacaggcgagcaacgcaaggagagctcaagagagaccacccctgcctccaggcccccgagttggacgagcacttggtggaaagagccactcaggaaagcaccttagaccgctggaaattccttcaagagcaaaacaaaacgaagcctgagttcaacgtcagaaaagtcgaaggtaccctgcctcccgacgtacttgtgattcatcaatcaaaatacaagtgtgggatgaagaaccatcatcctgaacagcaaagctccctgctaaacctctcttcgtcgaccccgacacatcaggagtccatgaacactggcacactcgcttccctgcgagggagggccaggagatccaaagggaagaacaaacacagcaagagggctctgcttgtgtgccagtga

SEQ ID NO: 70 - DNA, 7351 bp, Homo sapiens
  source 1..7351: mol_type = other DNA
aggccccacgcgccgccccgctcctccgccggatcgtctgtgggtgagtctcgagccaggaggctctgagccagtggcgattggctgacgcggtggctgcgcactcggcctgagaaactcggcaagcgcgcagtgtcgactccccggtctatgccaggcgcatctcagataccagcattgccaccggtgggtagaacactaagtgggctcttggagtccctgattccagaacttgactcttggatgacatttctggacctgctctgggccagagaggagaccacttcccttaagggaacgaggtctcactatattgcccagactggtctcgaactcctgggctcaaacagtccccctgcgttggcctcccaaagtgatggaattacaggtgtgaatcactgcatctgactatggcaaggatctctgtcactgagctaatccaaaagtaaatgagaaacttagaaaaagattgccaattccaaatcaacatatttagagaaaattggaaaaggagaagcttactacagctttatttgaggactttttaaagaacgctgggttctatctgtgagctgcaaatcttggagcaaaaaccagagacattgccagagcaaacaagaacagaaatacaaatggagaactggtcaaaagacataacccacagttatcttgaacaagaaactacggggataaataaaagtacgcagccagatgagcaactgactatgaattctgagaaaagtatgcatcggaaatccactgaattagttaatgaaataacatgtgagaacacagaatggccagggcagagatcaacgaattttcagatcatcagttcttatccagatgatgagtctgtttactgcactactgaaaaatacaacgttatggaacatagacataatgatatgcattatgaatgtatgactccttgtcaagttacttcagactcagataaagagaagacaatagcatttcttctaaaagaattggatattctcagaacaagcaataaaaagcttcagcagaaattggctaaagaagataaagaacagagaaaactaaagtttaagctggaactccaagagaaagaaacagaagctaaaattgctgaaaagacagcagctctggttgaagaagtgtattttgcgcagaaggaacgtgatgaagctgttatgtctagactgcaattagccattgaggagagagatgaagcaattgcacgagccaagcatatggaaatgtctctaaaagtgctagaaaatattaaccctgaagaaaatgacatgacattacaggaattactgaacagaataaacaatgcagacacagggatagctattcagaagaatggagctataattgtggatagaatctacaagaccaaggaatgtaaaatgagaataactgcagaagaaatgagtgcactaatagaagaacgggatgctgccttgtctaagtgcaaacggttagagcaggagcttcatcatgtgaaagagcagaaccagacttcagcaaacaacatgagacatctgactgctgaaaacaatcaagaacgtgctctgaaggcaaagttgttatctatgcaacaagccagagaaactgcagttcaacagtacaaaaaactggaagaggaaatccagacccttcgagtttactacagtttacacaaatctttatctcaagaagaaaatctgaaggatcagtttaactatacccttagtacatatgaagaagctttaaaaaacagagagaacattgtttccatcactcaacaacaaaatgaggaactggctactcaactgcaacaagctctgacagagcgagcaaatatggaattacaacttcaacatgccagagaggcctcccaagtggccaatgaaaaagttcaaaagttggaaaggctggtggatgtactgaggaagaaggttggaaccgggaccatgaggacagtgatctgattgaaaaaaaacgacagtctggggaagcgatcacatctggtgaccaggctgcttcattcaacactgtgtaaacaccaaagccttaacttagcaaacagttgttagaagtgggacactccaaccacattccaagctgagataaaatcaaatcacaaatgtttaaccactttgctgctgacttgagttatttatccaaatatattaactatagacttttaccaatgggtagctataaggttacagcttattttgtaactattttatatctcaatatctttaatataaatctttttactgagagatcattatagaaacatgttaaagttggttaggatcatatcttcacatatggccctttctgaatcaaagtgcggcaaagtaaatattgtctaagctttaatccactgtgttaggtcaaaacttcaaatacatgcatttttcaatatagggtatatttcttaactgatgagagaggcttagacatgagtgtgtagtcttccttcaatgcgtgtatgtaatctttgttagtataaaagatattaaatataggtgccaagaattaaatgtataatttgtttaataagagatggatatattaaaattacattcatcaaggcatgatttttgtttcactacaaataatgcaaactgttttcaataaaaagaggagactgttaatgtgtacttataaattcacattgtcagtattttttaatattgggtctgaataattatctgaattctacttaagctataagtctctgtcatttttgcttgaaaattagcatgcctctgtcttaaaagagaccatcaaacctattaagtattcttattgtttatctttttttaattgccatttgatttttattgtggaggaggaggatctaatatataatattcaatacaattgtaatgtagaaatataagaatttagaaaaaagtaaacttgccatttggttaaggttacctgctttttattttattccagaaataagatagttacagcatagcagtcatatgaagttatgaatagaactgagatttttatgtaattagttataggcaaaatactttcatatttttagattagacagacgaaagaccaagaggaaatgactgtgcctggaacaggatgaaatagacaagtagagatttaattagcaaaaattttgtagggaagtaaaattttttctaagtctatacgtttttaattcatctttaagattgagctaaattatctaccattgcctatttacaggataagtacattcaggacaatttattgtaccattctttcatataccatagcagattatccatttcaatttttttttactccacaggaaaatgtaagctactttgtcatagatcacaaaagaatcataatgctaacaaactctatttcttccttattaaatctgtatccattaaagtaacttttttaactatgagaattagaaaataagggacaacaggggttaaaaataaactgatttaatttgctctatagtcctaaagagaattatatcctctcatagaccaatgactctataatagagaaatatgtaatgatttggtcatgcatggagtcttgtctctgggctctatttgaaacgtgtaacagctccctatgtgaagaaacattatctttaaagtcatctgggaggtgcaggtaagtaaagagaaacaatttttgtcacaaagtagtccattcctgatctcacttaaaataaatcacaagtaaatttgaattttcggtttaatgttgaaagcagatgaacttttcttgagttattttgtcttttagaatacacaaaatatagtataaagacatttcacaatttccaaacaaatctttctacgcttaaatgatcaaattagaaaaaccaattcctataattaatatgcagaacttttatagaatatgatattataaagttaaatttgcaaaataattctaacatccactattgttcagtataggtaatctcccaaaaatatcacatcctcttgaaaatgaattgtctacaaaatttcaaatgcaaagtattacagccaaatattatcttaattaatttcttacacttaaaagtgtccatcaacagtgtgtcagatggtctttatatattttttctgtatgaaggaatagccttgcctactgaagataggttctctatgtattcaacaataatgcaactttagaagtattccacactgctgagaaatgttgctttgagtttgctttacacatcatcaactctaaatcctatagtaacatgagaattcacttcttcttgtaaaaataagtaatttacaggaaaggcaaaatgctaatactaacatttgtagcacttgatgatttacaaagtccttttacatctgttttttcatttgatcctatcgacaatcataataataataaattatatatataacccatataataacgatggtgatgataattttagtattactgtcattataattattattatattcattctacagatgaggaaacagactcagtaagccttggaatttgccaaggactggctaggacctagaactaagatcttataaccacactccatgcaactatgagtctctaaatctctctgtaagaaaaaaatttcaaagtaaagattttactcttgcaatttctgttgtgatagtaccatctctttcacatactacatacaaattccctaataatcaaaagattgtacacatttttttcaatgaagtacaataatgtgaactgcatgtgtaaacagcatcacattattaccacctctctatggtattatagtggcagtatgctttgcctcagccttcactaattaggaatattgtgacaattcattgctaataaaacataacatgagtctctttgtactgtttttaatttggcctgatgctaaaacctatcagcttagttttttaacatggtagtctaaacttcaaggcttgctctatctcctagagcaatttagagctaaaaccagtaataagcaaagttattggttataagcaaggtataagcagttaaagcaaagttattaattattaatatatgagaatttaaaattttataaataagttagaaattattaatgtattttaaatatggagtaaaatattttgctaatttttttcaatgataaagttgatcctttgtatatttccttattcaaataaattcagtcatcttcttatcaggttgcttatttatataatttgtttattttattaaaaatctagaatattaaagatttatacttttaatattgaatatagtttcgtagaaaaacattttatatgtactattcttaaagatcacaattatttttaaactttctattttcaaatacttgtgattaataaggcattctatgatgaaatagccatggcttatatatttttgtcttttcacatatgatgtgtttggaacccaaattttttactgattttattgtgttgttggtaactagtatttacagctaacctatctattcatattttacatatatatatatatatatacatgcacacacaccgatacatttaccttttaaaaaaatgatttataagtgaactgttaggctccagtgatttcagtgttttgttcatgttttattggaaatgcacgtggtttcttaggcttttaaacaaatacatggaatggttgaaagatttattttgctcgtgcttagctaaatatgtcatctctagaaaagatgtggtttgttttggcactgttttaaaaactcaaatattttaaacatttgttaagttggagcttgcacatttaaaccagcagcatacgttccagaagagcatctcaagttaccaaattttttgtaatccttttactagaaaaatcttttttgaagagtttttcttgtgcttgcttcggcagcatatatactgaaattagaaaaagaaaacttttcttttttattatatattttagtgtaagttaagttaaacttctaaaagttgaattatttaatgtaggacttcataaatagtatttgacaatgataaatgtgtatttttgagaatcattaaaatgtatacaatgatattcctttgcagaagtcttaatatgcataatttttaatctaattgtcttctaaatatagttttggtgtatgctggtattttagaagccaccaatttctggactatctgattattaacaaagatgtattttaatgcacaaactcaaatgtttaaaatatatatttactttgtactcaaataaaatcccctattgcaaatcctatacatatttcaatgcaattcttatacatcgatcttaatcaaatttataaatgtacaatcctgaagacagtgtactagatatgcactataaaatatattgcagtaaggaaacagattaagttcactgtcaaactgtctgacttccttaacaaatttaataaagataaatgtcaatatgaacaaatttgttaggtaccttaggaatctttcaaaactatttagatataaaataaaattatagtaataaaatattgaagagaaatatcatttttagtgaatatggttagaaagaacataagaagtgagatcaaattattttaattaaaaaaataggccaggtgcgatggctcacgcctgtaatcccagcattttgggaggccgaggcgggcggatcacaaggtcaggagattgagaccatcctggctaacacggtgaaaccccgtctctactaaaaaaagtacaaaaaagaagttagccgggcgtggtggcgggcgcctgtagtcccagctacttgggaggctgaggcaggagaatggcgtgaacccggaaggcagagcttgcagtgagccgagatcacgccattgcactccagcctgggtgacagagtgagactgtctcaaataataataataataataataataataataataataataatagactgactcaaatctaccataaagcacctctactcattttggaatatgtacgtggaattttgcttcaatagctgaaatctaaaaacactctttctgttgcatttgattattcaatcatgataaggtaaatgtaaacagactgtgtagcactctatcacctggagtgaacttagaatttacagcttagagtacatgactaaaagaggaatggagtggaagtgggcagtaggtgtctttggggcaaaaattctataaactggtgttatctaaactatcacattactttgttaaattattgtttcatcacgtatgacctttgtgatttgtcacacattttacttctacatatgcacataattgtaatttttttaactttaaaaagtcaattatgtttaagaaaatttttaaaataagaatgaacgtatgtgatatttactgtga

SEQ ID NO: 71 - DNA, 1187 bp, Homo sapiens
  source 1..1187: mol_type = other DNA
ccccagaggcagtggcaagaggaggtggcggcggtggtagtggtgggctcctgcatgaccgacctggtcagtcttacttctcgtttgccaaaaactggagaaaccatccatggacataagttttttattggctttggagggaaaggtgccaaccagtgtgtccaagctgctcggcttggagcaatgacgtccatggtgtgtaaggttggcaaagattcttttggcaatgattatatagaaaacttaaaacagaatgatatttctacagaatttacatatcagactaaagatgctgctacaggaactgcttctataattgtcaataatgaaggccagaatatcattgtcatagtggctggagcaaatttacttttgaatacggaggatctgagggcagcagccaatgtcattagcagagccaaagtcatggtctgccagctcgaaataactccagcaacttctttggaagccctaacaatggcccgcaggagtggagtgaaaaccttgttcaatccagcccctgccattgctgacctggatccccagttctacaccctctcagatgtgttctgctgcaatgaaagtgaggctgagattttaactggcctcacggtgggcagcgctgcagatgctggggaggctgcattagtgctcttgaaaaggggctgccaggtggtaatcattaccttaggggctgaaggatgtgtggtgctgtcacagacagaacctgagccaaagcacattcccacagagaaagtcaaggctgtggataccacgggtgctggtgacagctttgtgggagctctggccttctacctggcttactatccaaatctgtccttggaagacatgctcaacagatccaatttcattgcagcagtcagtgtccaggctgcaggaacacagtcatcttacccttacaaaaaagaccttccgcttactctgttttgattgctattagtcccaaaataaatatacctgggaataaaatgtacttgggggtggctgctcctggctaatgcttattagaaaatgtcctcgtcccctttctttgcaaatattagttcttttacgaagtcatcctcaagcttcaatttatttataacgatgattcttttgctttccatgcatttgcacaaaacaaccagaattaaagattccacaaccaagatctgtacaaacataaa

SEQ ID NO: 72 - DNA, 2415 bp, Homo sapiens
  source 1..2415: mol_type = other DNA
gtcatttgaagactctcttggaagagatagcgtcttgctgcaacctgcagtcccagcagaaaaaccttgtgatccttgttgcgggcgacatggaggacgactcactctacttgggaggtgagtggcagttcaaccacttttcaaaactcacatcttctcggccagatgcagcttttgctgaaatccagcggacttctctccctgagaagtcaccactctcatctgaggcccgtgtcgacctctgtgatgatttggctcctgtggcaagacagcttgctcccaggaagaagcttcctctgagtagcaggagacctgctgcggtgggggctgggctccagaatatgggaaatacctgctacgagaacgcttccctgcagtgcctgacatacacaccgccccttgccaactacatgctgtcccgggagcactctcaaacatgtcagcgtcccaagtgctgcatgctctgtactatgcaagctcacatcacatgggccctccacagtcctggtcatgtcatccagccctcacaggcattggctgctggcttccatagaggcaagcaggaagatgcccatgaatttctcatgttcactgtggatgccatgaaaaaggcatgccttcccggccacaagcaggtagatcatcactctaaggacaccaccctcatccaccaaatatttggaggctgctggagatctcaaatcaagtgtctccactgccacgggatttcagacacttttgacccttacctggacatcgccctggatatccaggcagctcagagtgtcaagcaagctttggaacagttggtgaagcccgaagaactcaatggagagaatgcctatcattgcggtctttgtctccagagggcgccggcctccaagacgttaactttacacacttctgccaaggtcctcatccttgtcttgaagagattctccgatgtcacaggcaacaaacttgccaagaatgtgcaatatcctgagtgccttgacatgcagccatacatgtctcagcagaacacaggacctcttgtctatgtcctctatgctgtgctggtccacgctgggtggagttgtcacgacggacattacttctcttatgtcaaagctcaagaaggccagtggtataaaatggatgatgccaaggtcactgcctgtagcatcacttctgtcctgagtcaacaggcctatgtcctcttttacatccagaagagtgaatgggaaagacacagtgagagtgtgtcaagaggcagggaaccaagagccctcggcgctgaagacacagacaggcgagcaacgcaaggagagctcaagagagaccacccctgcctccaggcacccgagttggacgagcgcttggtggaaagagccactcaggaaagcaccttagaccactggaaattcccccaagagcaaaacaaaacgaagcctgagttcaacgtcagaaaagtcgaaggtaccctgcctcccaacgtacttgtgattcatcaatcgaaatacaagtgtgggatgaaaaaccatcatcctgaacagcaaagctccctgctaaacctctcttcgacgacccggacagatcaggagtccgtgaacactggcaccctcgcttctctgcaagggaggaccaggagatccaaagggaagaacaaacacagcaagagggctctgcttgtgtgccagtgatctcagtggaagtgccgacccacacgtaggggtgaacgcacacacacacacgcacaaatacacccacaagcgcgcacgcaaacacacacacacacacaaacacgaacaccgtcaatcctacataaagtaatgaggagtccaagtttctgtctctacaacagggacaactggatagtgatggctgcatctcaggatgagcccacacatgggaaacatcaagttttggggtcgtgagtcttccgaacctctggagagactgtctgtgtgtttgtgttcatggtagatgacattcactgtgtatttctgaatatgacctactgacgtgtaggtttgagtgtgaggttattgcaggggactcggtttcctattttctcttggggtgtgtttcattcgtcagttgttgggcggcacgagaaggtgaaattttgctcatgtggcacatccatggatcattctcgccaccttgaatagtggaaactggaatgcatttagaagataggaacggtgctcttctttcttaccctggctcaccgtttttacattggtttctgaatggacctcaggcgccctgggacttgtgctcttgctggaacccacataacgccggaaacagacagaccgacttgcctgtttcacgatgtccaattccaatgagtcgaaatggaaaattttcccactggcatgtcagtcatttggaaataagtcgtattgataataaaggaaatcaaacaca

SEQ ID NO: 73 - DNA, 13992 bp, Homo sapiens
  source 1..13992: mol_type = other DNA
atcagttactgtgttgactcactcagtgttgggatcactcactttccccctacaggactcagatctgggaggcaattaccttcggagaaaaacgaataggaaaaactgaagtgttactttttttaaagctgctgaagtttgttggtttctcattgtttttaagcctactggagcaataaagtttgaagaacttttaccaggttttttttatcgctgccttgatatacacttttcaaaatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggattctgaaattaggaaaaggttggatgttgatataactgaacttcacagctggattactcgctcagaagctgtgttgcagagtcctgaatttgcaatctttcggaaggaaggcaacttctcagacttaaaagaaaaagtcaatgccatagagcgagaaaaagctgagaagttcagaaaactgcaagatgccagcagatcagctcaggccctggtggaacagatggtgaatgagggtgttaatgcagatagcatcaaacaagcctcagaacaactgaacagccggtggatcgaattctgccagttgctaagtgagagacttaactggctggagtatcagaacaacatcatcgctttctataatcagctacaacaattggagcagatgacaactactgctgaaaactggttgaaaatccaacccaccaccccatcagagccaacagcaattaaaagtcagttaaaaatttgtaaggatgaagtcaaccggctatcagatcttcaacctcaaattgaacgattaaaaattcaaagcatagccctgaaagagaaaggacaaggacccatgttcctggatgcagactttgtggcctttacaaatcattttaagcaagtcttttctgatgtgcaggccagagagaaagagctacagacaatttttgacactttgccaccaatgcgctatcaggagaccatgagtgccatcaggacatgggtccagcagtcagaaaccaaactctccatacctcaacttagtgtcaccgactatgaaatcatggagcagagactcggggaattgcaggctttacaaagttctctgcaagagcaacaaagtggcctatactatctcagcaccactgtgaaagagatgtcgaagaaagcgccctctgaaattagccggaaatatcaatcagaatttgaagaaattgagggacgctggaagaagctctcctcccagctggttgagcattgtcaaaagctagaggagcaaatgaataaactccgaaaaattcagaatcacatacaaaccctgaagaaatggatggctgaagttgatgtttttctgaaggaggaatggcctgcccttggggattcagaaattctaaaaaagcagctgaaacagtgcagacttttagtcagtgatattcagacaattcagcccagtctaaacagtgtcaatgaaggtgggcagaagataaagaatgaagcagagccagagtttgcttcgagacttgagacagaactcaaagaacttaacactcagtgggatcacatgtgccaacaggtctatgccagaaaggaggccttgaagggaggtttggagaaaactgtaagcctccagaaagatctatcagagatgcacgaatggatgacacaagctgaagaagagtatcttgagagagattttgaatataaaactccagatgaattacagaaagcagttgaagagatgaagagagctaaagaagaggcccaacaaaaagaagcgaaagtgaaactccttactgagtctgtaaatagtgtcatagctcaagctccacctgtagcacaagaggccttaaaaaaggaacttgaaactctaaccaccaactaccagtggctctgcactaggctgaatgggaaatgcaagactttggaagaagtttgggcatgttggcatgagttattgtcatacttggagaaagcaaacaagtggctaaatgaagtagaatttaaacttaaaaccactgaaaacattcctggcggagctgaggaaatctctgaggtgctagattcacttgaaaatttgatgcgacattcagaggataacccaaatcagattcgcatattggcacagaccctaacagatggcggagtcatggatgagctaatcaatgaggaacttgagacatttaattctcgttggagggaactacatgaagaggctgtaaggaggcaaaagttgcttgaacagagcatccagtctgcccaggagactgaaaaatccttacacttaatccaggagtccctcacattcattgacaagcagttggcagcttatattgcagacaaggtggacgcagctcaaatgcctcaggaagcccagaaaatccaatctgatttgacaagtcatgagatcagtttagaagaaatgaagaaacataatcaggggaaggaggctgcccaaagagtcctgtctcagattgatgttgcacagaaaaaattacaagatgtctccatgaagtttcgattattccagaaaccagccaattttgagcagcgtctacaagaaagtaagatgattttagatgaagtgaagatgcacttgcctgcattggaaacaaagagtgtggaacaggaagtagtacagtcacagctaaatcattgtgtgaacttgtataaaagtctgagtgaagtgaagtctgaagtggaaatggtgataaagactggacgtcagattgtacagaaaaagcagacggaaaatcccaaagaacttgatgaaagagtaacagctttgaaattgcattataatgagctgggagcaaaggtaacagaaagaaagcaacagttggagaaatgcttgaaattgtcccgtaagatgcgaaaggaaatgaatgtcttgacagaatggctggcagctacagatatggaattgacaaagagatcagcagttgaaggaatgcctagtaatttggattctgaagttgcctggggaaaggctactcaaaaagagattgagaaacagaaggtgcacctgaagagtatcacagaggtaggagaggccttgaaaacagttttgggcaagaaggagacgttggtggaagataaactcagtcttctgaatagtaactggatagctgtcacctcccgagcagaagagtggttaaatcttttgttggaataccagaaacacatggaaacttttgaccagaatgtggaccacatcacaaagtggatcattcaggctgacacacttttggatgaatcagagaaaaagaaaccccagcaaaaagaagacgtgcttaagcgtttaaaggcagaactgaatgacatacgcccaaaggtggactctacacgtgaccaagcagcaaacttgatggcaaaccgcggtgaccactgcaggaaattagtagagccccaaatctcagagctcaaccatcgatttgcagccatttcacacagaattaagactggaaaggcctccattcctttgaaggaattggagcagtttaactcagatatacaaaaattgcttgaaccactggaggctgaaattcagcagggggtgaatctgaaagaggaagacttcaataaagatatgaatgaagacaatgagggtactgtaaaagaattgttgcaaagaggagacaacttacaacaaagaatcacagatgagagaaagcgagaggaaataaagataaaacagcagctgttacagacaaaacataatgctctcaaggatttgaggtctcaaagaagaaaaaaggctctagaaatttctcatcagtggtatcagtacaagaggcaggctgatgatctcctgaaatgcttggatgacattgaaaaaaaattagccagcctacctgagcccagagatgaaaggaaaataaaggaaattgatcgggaattgcagaagaagaaagaggagctgaatgcagtgcgtaggcaagctgagggcttgtctgaggatggggccgcaatggcagtggagccaactcagatccagctcagcaagcgctggcgggaaattgagagcaaatttgctcagtttcgaagactcaactttgcacaaattcacactgtccgtgaagaaacgatgatggtgatgactgaagacatgcctttggaaatttcttatgtgccttctacttatttgactgaaatcactcatgtctcacaagccctattagaagtggaacaacttctcaatgctcctgacctctgtgctaaggactttgaagatctctttaagcaagaggagtctctgaagaatataaaagatagtctacaacaaagctcaggtcggattgacattattcatagcaagaagacagcagcattgcaaagtgcaacgcctgtggaaagggtgaagctacaggaagctctctcccagcttgatttccaatgggaaaaagttaacaaaatgtacaaggaccgacaagggcgatttgacagatctgttgagaaatggcggcgttttcattatgatataaagatatttaatcagtggctaacagaagctgaacagtttctcagaaagacacaaattcctgagaattgggaacatgctaaatacaaatggtatcttaaggaactccaggatggcattgggcagcggcaaactgttgtcagaacattgaatgcaactggggaagaaataattcagcaatcctcaaaaacagatgccagtattctacaggaaaaattgggaagcctgaatctgcggtggcaggaggtctgcaaacagctgtcagacagaaaaaagaggctagaagaacaaaagaatatcttgtcagaatttcaaagagatttaaatgaatttgttttatggttggaggaagcagataacattgctagtatcccacttgaacctggaaaagagcagcaactaaaagaaaagcttgagcaagtcaagttactggtggaagagttgcccctgcgccagggaattctcaaacaattaaatgaaactggaggacccgtgcttgtaagtgctcccataagcccagaagagcaagataaacttgaaaataagctcaagcagacaaatctccagtggataaaggtttccagagctttacctgagaaacaaggagaaattgaagctcaaataaaagaccttgggcagcttgaaaaaaagcttgaagaccttgaagagcagttaaatcatctgctgctgtggttatctcctattaggaatcagttggaaatttataaccaaccaaaccaagaaggaccatttgacgttaaggaaactgaaatagcagttcaagctaaacaaccggatgtggaagagattttgtctaaagggcagcatttgtacaaggaaaaaccagccactcagccagtgaagaggaagttagaagatctgagctctgagtggaaggcggtaaaccgtttacttcaagagctgagggcaaagcagcctgacctagctcctggactgaccactattggagcctctcctactcagactgttactctggtgacacaacctgtggttactaaggaaactgccatctccaaactagaaatgccatcttccttgatgttggaggtacctgctctggcagatttcaaccgggcttggacagaacttaccgactggctttctctgcttgatcaagttataaaatcacagagggtgatggtgggtgaccttgaggatatcaacgagatgatcatcaagcagaaggcaacaatgcaggatttggaacagaggcgtccccagttggaagaactcattaccgctgcccaaaatttgaaaaacaagaccagcaatcaagaggctagaacaatcattacggatcgaattgaaagaattcagaatcagtgggatgaagtacaagaacaccttcagaaccggaggcaacagttgaatgaaatgttaaaggattcaacacaatggctggaagctaaggaagaagctgagcaggtcttaggacaggccagagccaagcttgagtcatggaaggagggtccctatacagtagatgcaatccaaaagaaaatcacagaaaccaagcagttggccaaagacctccgccagtggcagacaaatgtagatgtggcaaatgacttggccctgaaacttctccgggattattctgcagatgataccagaaaagtccacatgataacagagaatatcaatgcctcttggagaagcattcataaaagggtgagtgagcgagaggctgctttggaagaaactcatagattactgcaacagttccccctggacctggaaaagtttcttgcctggcttacagaagctgaaacaactgccaatgtcctacaggatgctacccgtaaggaaaggctcctagaagactccaagggagtaaaagagctgatgaaacaatggcaagacctccaaggtgaaattgaagctcacacagatgtttatcacaacctggatgaaaacagccaaaaaatcctgagatccctggaaggttccgatgatgcagtcctgttacaaagacgtttggataacatgaacttcaagtggagtgaacttcggaaaaagtctctcaacattaggtcccatttggaagccagttctgaccagtggaagcgtctgcacctttctctgcaggaacttctggtgtggctacagctgaaagatgatgaattaagccggcaggcacctattggaggcgactttccagcagttcagaagcagaacgatgtacatagggccttcaagagggaattgaaaactaaagaacctgtaatcatgagtactcttgagactgtacgaatatttctgacagagcagcctttggaaggactagagaaactctaccaggagcccagagagctgcctcctgaggagagagcccagaatgtcactcggcttctacgaaagcaggctgaggaggtcaatactgagtgggaaaaattgaacctgcactccgctgactggcagagaaaaatagatgagacccttgaaagactccgggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgggatgctgagctcattgctgaggccaagctactgcgtcaacacaaaggccgcctggaagccaggatgcaaatcctggaagaccacaataaacagctggagtcacagttacacaggctaaggcagctgctggagcaaccccaggcagaggccaaagtgaatggcacaacggtgtcctctccttctacctctctacagaggtccgacagcagtcagcctatgctgctccgagtggttggcagtcaaacttcggactccatgggtgaggaagatcttctcagtcctccccaggacacaagcacagggttagaggaggtgatggagcaactcaacaactccttccctagttcaagaggaagaaatacccctggaaagccaatgagagaggacacaatgtaggaagtcttttccacatggcagatgatttgggcagagcgatggagtccttagtatcagtcatgacagatgaagaaggagcagaataaatgttttacaactcctgattcccgcatggtttttataatattcatacaacaaagaggattagacagtaagagtttacaagaaataaatctatatttttgtgaagggtagtggtattatactgtagatttcagtagtttctaagtctgttattgttttgttaacaatggcaggttttacacgtctatgcaattgtacaaaaaagttataagaaaactacatgtaaaatcttgatagctaaataacttgccatttctttatatggaacgcattttgggttgtttaaaaatttataacagttataaagaaagattgtaaactaaagtgtgctttataaaaaaaagttgtttataaaaacccctaaaaacaaaacaaacacacacacacacacatacacacacacacacaaaactttgaggcagcgcattgttttgcatccttttggcgtgatatccatatgaaattcatggctttttctttttttgcatattaaagataagacttcctctaccaccacaccaaatgactactacacactgctcatttgagaactgtcagctgagtggggcaggcttgagttttcatttcatatatctatatgtctataagtatataaatactatagttatatagataaagagatacgaatttctatagactgactttttccattttttaaatgttcatgtcacatcctaatagaaagaaattacttctagtcagtcatccaggcttacctgcttggtctagaatggatttttcccggagccggaagccaggaggaaactacaccacactaaaacattgtctacagctccagatgtttctcattttaaacaactttccactgacaacgaaagtaaagtaaagtattggatttttttaaagggaacatgtgaatgaatacacaggacttattatatcagagtgagtaatcggttggttggttgattgattgattgattgatacattcagcttcctgctgctagcaatgccacgatttagatttaatgatgcttcagtggaaatcaatcagaaggtattctgaccttgtgaacatcagaaggtattttttaactcccaagcagtagcaggacgatgatagggctggagggctatggattcccagcccatccctgtgaaggagtaggccactctttaagtgaaggattggatgattgttcataatacataaagttctctgtaattacaactaaattattatgccctcttctcacagtcaaaaggaactgggtggtttggtttttgttgcttttttagatttattgtcccatgtgggatgagtttttaaatgccacaagacataatttaaaataaataaactttgggaaaaggtgtaaaacagtagccccatcacatttgtgatactgacaggtatcaacccagaagcccatgaactgtgtttccatcctttgcatttctctgcgagtagttccacacaggtttgtaagtaagtaagaaagaaggcaaattgattcaaatgttacaaaaaaacccttcttggtggattagacaggttaaatatataaacaaacaaacaaaaattgctcaaaaaagaggagaaaagctcaagaggaaaagctaaggactggtaggaaaaagctttactctttcatgccattttatttctttttgatttttaaatcattcattcaatagataccaccgtgtgacctataattttgcaaatctgttacctctgacatcaagtgtaattagcttttggagagtgggctgacatcaagtgtaattagcttttggagagtgggttttgtccattattaataattaattaattaacatcaaacacggcttctcatgctatttctacctcactttggttttggggtgttcctgataattgtgcacacctgagttcacagcttcaccacttgtccattgcgttattttctttttcctttataattctttctttttccttcataattttcaaaagaaaacccaaagctctaaggtaacaaattaccaaattacatgaagatttggtttttgtcttgcatttttttcctttatgtgacgctggaccttttctttacccaaggatttttaaaactcagatttaaaacaaggggttactttacatcctactaagaagtttaagtaagtaagtttcattctaaaatcagaggtaaatagagtgcataaataattttgttttaatctttttgtttttcttttagacacattagctctggagtgagtctgtcataatatttgaacaaaaattgagagctttattgctgcattttaagcataattaatttggacattatttcgtgttgtgttctttataaccaccaagtattaaactgtaaatcataatgtaactgaagcataaacatcacatggcatgttttgtcattgttttcaggtactgagttcttacttgagtatcataatatattgtgttttaacaccaacactgtaacatttacgaattatttttttaaacttcagttttactgcattttcacaacatatcagacttcaccaaatatatgccttactattgtattatagtactgctttactgtgtatctcaataaagcacgcagttatgttacaaaaaa

SEQ ID NO: 74 - protein, 81 aa, Homo sapiens
MLRQIIGQAKKHPSLIPLFVFIGTGATGATLYLLRLALFNPDVCWDRNNPEPWNKLGPNDQYKFYSVNVDYSKLKKERPDF

SEQ ID NO: 75 - protein, 212 aa, Homo sapiens
MEISMPPPQIYVEKTLAIIKPDIVDKEEEIQDIILRSGFTIVQRRKLRLSPEQCSNFYVEKYGKMFFPNLTAYMSSGPLVAMILARHKAISYWLELLGPNNSLVAKETHPDSLRAIYGTDDLRNALHGSNDFAAAEREIRFMFPEVIVEPIPIGQAAKDYLNLHIMPTLLEGLTELCKQKPADPLIWLADWLLKNNPNKPKLCHHPIVEEPY

SEQ ID NO: 76 - protein, 412 aa, Homo sapiens
MGLHLRPYRVGLLPDGLLFLLLLLMLLADPALPAGRHPPVVLVPGDLGNQLEAKLDKPTVVHYLCSKKTESYFTIWLNLELLLPVIIDCWIDNIRLVYNKTSRATQFPDGVDVRVPGFGKTFSLEFLDPSKSSVGSYFHTMVESLVGWGYTRGEDVRGAPYDWRRAPNENGPYFLALREMIEEMYQLYGGPVVLVAHSMGNMYTLYFLQRQPQAWKDKYIRAFVSLGAPWGGVAKTLRVLASGDNNRIPVIGPLKIREQQRSAVSTSWLLPYNYTWSPEKVFVQTPTINYTLRDYRKFFQDIGFEDGWLMRQDTEGLVEATMPPGVQLHCLYGTGVPTPDSFYYESFPDRDPKICFGDGDGTVNLKSALQCQAWQSRQEHQVLLQELPGSEHIEMLANATTLAYLKRVLLGP

SEQ ID NO: 77 - protein, 67 aa, Homo sapiens
MEEWDVPQMKKEVESLKYQLAFQREMASKTIPELLKWIEDGIPKDPFLNPDLMKNNPWVEKGKCTIL

SEQ ID NO: 78 - protein, 299 aa, Homo sapiens
MLRLFYFSAIIASVILNFVGIIMNLFITVVNCKTWVKSHRISSSDRILFSLGITRFLMLGLFLVNTIYFVSSNTERSVYLSAFFVLCFMFLDSSSVWFVTLLNILYCVKITNFQHSVFLLLKRNISPKIPRLLLACVLISAFTTCLYITLSQASPFPELVTTRNNTSFNISEGILSLVVSLVLSSSLQFIINVTSASLLIHSLRRHIQKMQKNATGFWNPQTEAHVGAMKLMVYFLILYIPYSVATLVQYLPFYAGMDMGTKSICLIFATLYSPGHSVLIIITHPKLKTTAKKILCFKK

SEQ ID NO: 79 - protein, 462 aa, Homo sapiens
MARVLGAPVALGLWSLCWSLAIATPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 80 - protein, 263 aa, Homo sapiens
MMPKHCFLGFLISFFLTGVAGTQSTHESLKPQRVQFQSRNFHNILQWQPGRALTGNSSVYFVQYKIMFSCSMKSSHQKPSGCWQHISCNFPGCRTLAKYGQRQWKNKEDCWGTQELSCDLTSETSDIQEPYYGRVRAASAGSYSEWSMTPRFTPWWETKIDPPVMNITQVNGSLLVILHAPNLPYRYQKEKNVSIEDYYELLYRVFIINNSLEKEQKVYEGAHRAVEIEALTPHSSYCVVAEIYQPMLDRRSQRSEERCVEIP

SEQ ID NO: 81 - protein, 483 aa, Homo sapiens
MTKMDIRGAVDAAVPTNIIAAKAAEVRANKVNWQSYLQGQMISAEDCEFIQRFEMKRSPEEKQEMLQTEGSQCAKTFINLMTHICKEQTVQYILTMVDDMLQENHQRVSIFFDYARCSKNTAWPYFLPMLNRQDPFTVHMAARIIAKLAAWGKELMEGSDLNYYFNWIKTQLSSQKLRGSGVAVETGTVSSSDSSQYVQCVAGCLQLMLRVNEYRFAWVEADGVNCIMGVLSNKCGFQLQYQMIFSIWLLAFSPQMCEHLRRYNIIPVLSDILQESVKEKVTRIILAAFRNFLEKSTERETRQEYALAMIQCKVLKQLENLEQQKYDDEDISEDIKFLLEKLGESVQDLSSFDEYSSELKSGRLEWSPVHKSEKFWRENAVRLNEKNYELLKILTKLLEVSDDPQVLAVAAHDVGEYVRHYPRGKRVIEQLGGKQLVMNHMHHEDQQVRYNALLAVQKLMVHNWEYLGKQLQSEQPQTAAARS

SEQ ID NO: 82 - protein, 636 aa, Homo sapiens
MKKLQGAHLRKPVTPDLLMTPSDQGDVDLDVDFAAHRGNWTGKLDFLLSCIGYCVGLGNVWRFPYRAYTNGGGAFLVPYFLMLAICGIPLFFLELSLGQFSSLGPLAVWKISPLFKGAGAAMLLIVGLVAIYYNMIIAYVLFYLFASLTSDLPWEHCGNWWNTELCLEHRVSKDGNGALPLNLTCTVSPSEEYWSRYVLHIQGSQGIGSPGEIRWNLCLCLLLAWVIVFLCILKGVKSSGKVVYFTATFPYLILLMLLVRGVTLPGAWKGIQFYLTPQFHHLLSSKVWIEAALQIFYSLGVGFGGLLTFASYNTFHQNIYRDTFIVTLGNAITSILAGFAIFSVLGYMSQELGVPVDQVAKAGPGLAFVVYPQAMTMLPLSPFWSFLFFFMLLTLGLDSQFAFLETIVTAVTDEFPYYLRPKKAVFSGLICVAMYLMGLILTTDGGMYWLVLLDDYSASFGLMVVVITTCLAVTRVYGIQRFCRDIHMMLGFKPGLYFRACWLFLSPATLLALMVYSIVKYQPSEYGSYRFPPWAELLGILMGLLSCLMIPAGMLVAVLREEGSLWERLQQASRPAMDWGPSLEENRTGMYVATLAGSQSPKPLMVHMRKYGGITSFENTAIEVDREIAEEEESMM

SEQ ID NO: 83 - protein, 1896 aa, Homo sapiens
MKASSGDQGSPPCFLRFPRPVRVVSGAEAELKCVVLGEPPPVVVWEKGGQQLAASERLSFPADGAEHGLLLTAALPTDAGVYVCRARNAAGEAYAAAAVTVLEPPASDPELQPAERPLPSPGSGEGAPVFLTGPRSQWVLRGAEVVLTCRAGGLPEPTLYWEKDGMALDEVWDSSHFALQPGRAEDGPGASLALRILAARLPDSGVYVCHARNAHGHAQAGALLQVHQPPESPPADPDEAPAPVVEPLKCAPKTFWVNEGKHAKFRCYVMGKPEPEIEWHWEGRPLLPDRRRLMYRDRDGGFVLKVLYCQAKDRGLYVCAARNSAGQTLSAVQLHVKEPRLRFTRPLQDVEGREHGIAVLECKVPNSRIPTAWFREDQRLLPCRKYEQIEEGTVRRLIIHRLKADDDGIYLCEMRGRVRTVANVTVKGPILKRLPRKLDVLEGENAVLLVETLEAGVEGRWSRDGEELPVICQSSSGHMHALVLPGVTREDAGEVTFSLGNSRTTTLLRVKCVKHSPPGPPILAEMFKGHKNTVLLTWKPPEPAPETPFIYRLERQEVGSEDWIQCFSIEKAGAVEVPGDCVPSEGDYRFRICTVSGHGRSPHVVFHGSAHLVPTARLVAGLEDVQVYDGEDAVFSLDLSTIIQGTWFLNGEELKSNEPEGQVEPGALRYRIEQKGLQHRLILHAVKHQDSGALVGFSCPGVQDSAALTIQESPVHILSPQDRVSLTFTTSERVVLTCELSRVDFPATWYKDGQKVEESELLVVKMDGRKHRLILPEAKVQDSGEFECRTEGVSAFFGVTVQDPPVHIVDPREHVFVHAITSECVMLACEVDREDAPVRWYKDGQEVEESDFVVLENEGPHRRLVLPATQPSDGGEFQCVAGDECAYFTVTITDVSSWIVYPSGKVYVAAVRLERVVLTCELCRPWAEVRWTKDGEEVVESPALLLQKEDTVRRLVLPAVQLEDSGEYLCEIDDESASFTVTVTEPPVRIIYPRDEVTLIAVTLECVVLMCELSREDAPVRWYKDGLEVEESEALVLERDGPRCRLVLPAAQPEDGGEFVCDAGDDSAFFTVTVTAPPERIVHPAARSLDLHFGAPGRVELRCEVAPAGSQVRWYKDGLEVEASDALQLGAEGPTRTLTLPHAQPEDAGEYVCETRHEAITFNVILAEPPVQFLALETTPSPLCVAPGEPVVLSCELSRAGAPVVWSHNGRPVQEGEGLELHAEGPRRVLCIQAAGPAHAGLYTCQSGAAPGAPSLSFTVQVAEPPVRVVAPEAAQTRVRSTPGGDLELVVHLSGPGGPVRWYKDGERLASQGRVQLEQAGARQVLRVQGARSGDAGEYLCDAPQDSRIFLVSVEEPLLVKLVSELTPLTVHEGDDATFRCEVSPPDADVTWLRNGAVVTPGPQVEMAQNGSSRILTLRGCQLGDAGTVTLRAGSTATSARLHVRETELLFLRRLQDVRAEEGQDVCLEVETGRVGAAGAVRWVRGGQPLPHDSRLSMAQDGHIHRLFIHGVILADQGTYGCESHHDRTLARLSVRPRQLRVLRPLEDVTISEGGSATFQLELSQEGVTGEWARGGVQLYPGPKCHIHSDGHRHRLVLNGLGLADSGCVSFTADSLRCAARLIVREVPVTIVRGPHDLEVTEGDTATFECELSQALADVTWEKDGNALTPSPRLRLQALGTRRLLQLRRCGPSDAGTYSCAVGTARAGPVRLTVRERTVAVLSELRSVSAREGDGATFECTVSEVETTGRWELGGRPLRPGARVRIRQEGKKHILVLSELRAEDAGEVRFQAGPAQSLALLEVEALPLQMCRHPPREKTVLVGRRAVLEVTVSRSGGHVCWLREGAELCPGDKYEMRSHGPTHSLVIHDVRPEDQGTYCCQAGQDSTHTRLLVEGN

SEQ ID NO: 84 - protein, 275 aa, Homo sapiens
MQLQVFWTGLEYTCRLLGITTAAVLIGVGTETFLQGQFKSLAFYLLFTGAAVSICEGAYFVAQLLAICFQCQPGSLADRVREKAHWLGCFQKFLAYLLLSVACFLHPVLVWHVTIPGSMLIITGLAYFLLSKRKKRKAAPEVLASPEQYTDPSSSAVSTTGSGDTEQTYTFHGALKEGPSSLFIHMKSILKGTKKPSALQPPNTLMELSLEPADSLAKKKQVHFEDNLVRIVPSLAEGLDDGDSEPEETTSDTTPIIPPPQAPLFLSSLTATGLF

SEQ ID NO: 85 - protein, 480 aa, Homo sapiens
MEVAPEQPRWMAHPAVLNAQHPDSHHPGLAHNYMEPAQLLPPDEVDVFFNHLDSQGNPYYANPAHARARVSYSPAHARLTGGQMCRPHLLHSPGLPWLDGGKAALSAAAAHHHNPWTVSPFSKTPLHPSAAGGPGGPLSVYPGAGGGSGGGSGSSVASLTPTAAHSGSHLFGFPPTPPKEVSPDPSTTGAASPASSSAGGSAARGEDKDGVKYQVSLTESMKMESGSPLRPGLATMGTQPATHHPIPTYPSYVPAAAHDYSSGLFHPGGFLGGPASSFTPKQRSKARSCSEGRECVNCGATATPLWRRDGTGHYLCNACGLYHKMNGQNRPLIKPKRRLSAARRAGTCCANCQTTTTTLWRRNANGDPVCNACGLYYKLHNVNRPLTMKKEGIQTRNRKMSNKSKKSKKGAECFEELSKCMQEKSSPFSAAALAGHMAPVGHLPPFSHSGHILPTPTPIHPSSSLSFGHPHPSSMVTAMG

SEQ ID NO: 86 - protein, 530 aa, Homo sapiens
MEDDSLYLGGEWQFNHFSKLTSSRPDAAFAEIQRTSLPEKSPLSCETRVDLCDDLAPVARQLAPREKPPLSSRRPAAVGAGLQNMGNTCYVNASLQCLTYKPPLANYMLFREHSQTCHRHKGCMLCTMQAHITRALHIPGHVIQPSQALAAGFHRGKQEDAHEFLMFTVDAMRKACLPGHKQVDRHSKDTTLIHQIFGGYWRSQIKCLHCHGISDTFDPYLDIALDIQAAQSVQQALEQLVKPEELNGENAYHCGVCLQRAPASKTLTLHNSAKVLILVLKRFPDVTGNKIAKNVQYPECLDMQPYMSQQNTGPLVYVLYAVLVHAGWSCHNGHYSSYVKAQEGQWYKMDDAEVTASSITSVLSQQAYVLFYIQKSEWERHSESVSRGREPRALGVEDTDRRATQGELKRDHPCLQAPELDEHLVERATQESTLDHWKFLQEQNKTKPEFNVRRVEGTVPPDVLVIHQSKYKCRMKNHHPEQQSSLLNLSSTTPTDQESMNTGTLASLRGRTRRSKGKNKHSKRALLVCQ

SEQ ID NO: 87 - protein, 530 aa, Homo sapiens
MEDDSLYLGGDWQFNHFSKLTSSRLDAAFAEIQRTSLSEKSPLSSETRFDLCDDLAPVARQLAPREKLPLSSRRPAAVGAGLQKIGNTFYVNVSLQCLTYTLPLSNYMLSREDSQTCHLHKCCMFCTMQAHITWALHSPGHVIQPSQVLAAGFHRGEQEDAHEFLMFTVDAMKKACLPGHKQLDHHSKDTTLIHQIFGAYWRSQIKYLHCHGVSDTFDPYLDIALDIQAAQSVKQALEQLVKPKELNGENAYHCGLCLQKAPASKTLTLPTSAKVLILVLKRFSDVTGNKLAKNVQYPKCRDMQPYMSQQNTGPLVYVLYAVLVHAGWSCHNGHYFSYVKAQEGQWYKMDDAEVTASGITSVLSQQAYVLFYIQKSEWERHSESVSRGREPRALGAEDTDRPATQGELKRDHPCLQVPELDEHLVERATQESTLDHWKFPQEQNKTKPEFNVRKVEGTLPPNVLVIHQSKYKCGMKNHHPEQQSSLLNLSSTKPTDQESMNTGTLASLQGSTRRSKGNNKHSKRSLLVCQ

SEQ ID NO: 88 - protein, 384 aa, Homo sapiens
MEGLGRSCLWLRRELSPPRPRLLLLDCRSRELYESARIGGALSVALPALLLRRLRRGSLSVRALLPGPPLQPPPPAPVLLYDQGGGRRRRGEAEAEAEEWEAESVLGTLLQKLREEGYLAYYLQGGFSRFQAECPHLCETSLAGRAGSSMAPVPGPVPVVGLGSLCLGSDCSDAESEADRDSMSCGLDSEGATPPPVGLRASFPVQILPNLYLGSARDSANLESLAKLGIRYILNVTPNLPNFFEKNGDFHYKQIPISDHWSQNLSRFFPEAIEFIDEALSQNCGVLVHCLAGVSRSVTVTVAYLMQKLHLSLNDAYDLVKRKKSNISPNFNFMGQLLDFERSLRLEERHSQEQGSGGQASAASNPPSFFTTPTSDGAFELAPT

SEQ ID NO: 89 - protein, 237 aa, Homo sapiens
MALPPSPLAMEYVNDFDLMKFEVKREPSEGRPGPPTASLGSTPYSSVPPSPTFSEPGMVGATEGTRPGLEELYWLATLQQQLGAGEALGLSPEEAMELLQGQGPVPVDGPHGYYPGSPEETGAQHVQLAERFSDAALVSMSVRELNRQLRGCGRDEALRLKQRRRTLKNRGYAQACRSKRLQQRRGLEAERARLAAQLDALRAEVARLARERDLYKARCDRLTSSGPGSGDPSHLFL

SEQ ID NO: 90 - protein, 1399 aa, Homo sapiens
MSGLGDSSSDPANPDSHKRKGSPCDTLASSTEKRRREQENKYLEELAELLSANISDIDSLSVKPDKCKILKKTVDQIQLMKRMEQEKSTTDDDVQKSDISSSSQGVIEKESLGPLLLEALDGFFFVVNCEGRIVFVSENVTSYLGYNQEELMNTSVYSILHVGDHAEFVKNLLPKSLVNGVPWPQEATRRNSHTFNCRMLIHPPDEPGTENQEACQRYEVMQCFTVSQPKSIQEDGEDFQSCLICIARRLPRPPAITGVESFMTKQDTTGKIISIDTSSLRAAGRTGWEDLVRKCIYAFFQPQGREPSYARQLFQEVMTRGTASSPSYRFILNDGTMLSAHTKCKLCYPQSPDMQPFIMGIHIIDREHSGLSPQDDTNSGMSIPRVNPSVNPSISPAHGVARSSTLPPSNSNMVSTRINRQQSSDLHSSSHSNSSNSQGSFGCSPGSQIVANVALNQGQASSQSSNPSLNLNNSPMEGTGISLAQFMSPRRQVTSGLATRPRMPNNSFPPNISTLSSPVGMTSSACNNNNRSYSNIPVTSLQGMNEGPNNSVGFSASSPVLRQMSSQNSPSRLNIQPAKAESKDNKEIASILNEMIQSDNSSSDGKPLDSGLLHNNDRLSDGDSKYSQTSHKLVQLLTTTAEQQLRHADIDTSCKDVLSCTGTSNSASANSSGGSCPSSHSSLTERHKILHRLLQEGSPSDITTLSVEPDKKDSASTSVSVTGQVQGNSSIKLELDASKKKESKDHQLLRYLLDKDEKDLRSTPNLSLDDVKVKVEKKEQMDPCNTNPTPMTKPTPEEIKLEAQSQFTADLDQFDQLLPTLEKAAQLPGLCETDRMDGAVTSVTIKSEILPASLQSATARPTSRLNRLPELELEAIDNQFGQPGTGDQIPWTNNTVTAINQSKSEDQCISSQLDELLCPPTTVEGRNDEKALLEQLVSFLSGKDETELAELDRALGIDKLVQGGGLDVLSERFPPQQATPPLIMEERPNLYSQPYSSPSPTANLPSPFQGMVRQKPSLGTMPVQVTPPRGAFSPGMGMQPRQTLNRPPAAPNQLRLQLQQRLQGQQQLIHQNRQAILNQFAATAPVGINMRSGMQQQITPQPPLNAQMLAQRQRELYSQQHRQRQLIQQQRAMLMRQQSFGNNLPPSSGLPVQMGNPRLPQGAPQQFPYPPNYGTNPGTPPASTSPFSQLAANPEASLANRNSMVSRGMTGNIGGQFGTGINPQMQQNVFQYPGAGMVPQGEANFAPSLSPGSSMVPMPIPPPQSSLLQQTPPASGYQSPDMKAWQQGAIGNNNVFSQAVQNQPTPAQPGVYNNMSITVSMAGGNTNVQNMNPMMAQMQMSSLQMPGMNTVCPEQINDPALRHTGLYCNQLSSTDLLKTEADGTQDKKTEEFFSVVTTD

SEQ ID NO: 91 - protein, 319 aa, Homo sapiens
MDPQNQHGSGSSLVVIQQPSLDSRQRLDYEREIQPTAILSLDQIKAIRGSNEYTEGPSVVKRPAPRTAPRQEKHERTHEIIPINVNNNYEHRHTSHLGHAVLPSNARGPILSRSTSTGSAASSGSNSSASSEQGLLGRSPPTRPVPGHRSERAIRTQPKQLIVDDLKGSLKEDLTQHKFICEQCGKCKCGECTAPRTLPSCLACNRQCLCSAESMVEYGTCMCLVKGIFYHCSNDDEGDSYSDNPCSCSQSHCCSRYLCMGAMSLFLPCLLCYPPAKGCLKLCRRCYDWIHRPGCRCKNSNTVYCKLESCPSRGQGKPS

SEQ ID NO: 92 - DNA, 1593 bp, Homo sapiens
  source 1..1593: mol_type = other DNA
atggaggacgactcactctacttgggaggtgagtggcagttcaaccacttttcaaaactcacatcttctcggccagatgcagcttttgctgaaatccagcgtacttctctccctgagaagtcaccactctcatgtgagacccgtgtcgacctctgtgatgatttggctcctgtggcaagacagcttgctcccagggagaagcctcctctgagtagcaggagacctgctgcggtgggggctgggctccagaatatgggaaatacctgctacgtgaacgcttccctgcagtgcctgacatacaaaccgccacttgccaactacatgctgttccgggagcactctcaaacgtgtcatcgtcacaagggctgcatgctctgtactatgcaagctcacatcacaagggccctccacattcctggccatgtcatccagccctcacaggcattggctgctggcttccatagaggcaagcaggaagatgcccatgaatttctcatgttcactgtggatgccatgagaaaggcatgccttcccgggcacaagcaggtagatcgtcactctaaggacaccaccctcatccaccaaatatttggaggctactggagatctcaaatcaagtgtctccactgccacggcatttcagacacttttgacccttacctggacatcgccctggatatccaggcagctcagagtgtccagcaagctttggaacagttggtgaagcccgaagaactcaatggagagaatgcctatcattgtggtgtttgtctccagagggcgccggcctccaagacgttaactttacacaactctgccaaggtcctcatccttgtattgaagagattccccgatgtcacaggcaacaaaattgccaagaatgtgcaatatcctgagtgccttgacatgcagccatacatgtctcagcagaacacaggacctctcgtctatgtcctctatgctgtgctggtccacgctgggtggagttgtcacaacggacattactcctcttatgtcaaagctcaagaaggccagtggtataaaatggatgatgccgaggtcaccgcctctagcatcacttctgtcctgagtcaacaggcctacgtcctcttttacatccagaagagtgaatgggaaagacacagtgagagtgtgtcaagaggcagggaaccaagagcccttggcgtagaagacacagacaggcgagcaacgcaaggagagctcaagagagaccacccctgcctccaggcccccgagttggacgagcacttggtggaaagagccactcaggaaagcaccttagaccactggaaattccttcaagagcaaaacaaaacgaagcctgagttcaacgtcagaagagtcgaaggtacggtgcctcccgacgtacttgtgattcatcaatcaaaatacaagtgtcggatgaagaaccatcatcctgaacagcaaagctccctgctaaacctctcttcgacgaccccgacagatcaggagtccatgaacactggcacactcgcttccctacgagggaggaccaggagatccaaagggaagaacaaacacagcaagagggctctgcttgtgtgccagtga

SEQ ID NO: 93 - DNA, 2657 bp, Homo sapiens
  source 1..2657: mol_type = other DNA
agcttcccggctgctttgtttacctaatcaagcctgggcaatggcgggctcccctcccccagcctcgctgctgccttgcagtttgatctcagactgctgtgctagcaatcagcgagactccgtgggcgtaggaccctccgagccaggtgtgggatatagtctcgtggtgcgccgttttttaagcccgtcggaaaagcgcagtattcgggtgggagtgacccgattttccaggtgcgtccgtcacccctttctttgactcggaaagggaactccctgaccccttgcgcttcccaagtgaggcaatgcctcgccctgcttcggctcgcgcacggtgcgcgcacccactgacctgcgcccactgtctggcactccctagagagatgaacccggtacctcagatggaaatgcagaaatcaccgtcttctgcgtcgctcacgctgggagctgtagaccagagctgttcctattcggccatcttggctccttattctcattaccttcttgagttgtggctcagacactacaacagagctctgtcacctctttctaaaagctgggaaatggttattgtcaacgccagccccattttggaggcacctggttgcaggggaccacagctagctgggtgcggtggctcaggactgtaattccagctacatgagaggattgcctgaggccaggagtttgagaccagcctgaacaacataatagactgggagctccatgaggtccgagacaacctgaagacgcccagctcagcagagactggctacggcaatcactcagtaactatttgttgaattggacataatagaagagctgaagaattttaaacaagtcaccatcagtgcagtggattcctcaaagcaaacctggaaatctcggcatgagttggatttaaggcagtacattttcagctcctgaaaatttgaacagtttctcaagagaacaacttgaaacagaaaggcaaggatgtagatgtggttgtagaagatgatgtctgcggccaccaaactgtccactgtgaagacaaaccatgcatgtttagtaggcagtggctcctcttccctgtggggagtcccactgcttccctggcctgggtgttctctgccggtagatgttattgacaaaataagcaaagtcctctgattctgagaattagagttaatgatagctctagattgcctctgccatgctatttaaagtcctgctttattgtctgacatgagttcggcatctatgaggacttgtccagtgcaaagtacaatctccactgaaagatatcaccagcacatcttgtcactgtgctgtagcaagctgggcagtacaatgatgttggcaggatcctgtttaggtggaggatggccacctgggcatcaagaatcttctaccctgattccacattgactccccaaagtgggcttcagacagctctttacttacgtcttctgattaaagcagcagacagggtcccagctagagaaaagttctgggagcaggagaggtgttggtggctttaaaaccaggggtgaagattgaattaggaggttaggcaggagtaggatcaaagacttagaaaatgcttgtttcctcctattattcttatgagagctgatacggggctttcagttttgggggagagccttggaagagaatgagggaatttaatcaactagcgggacctttccaacttacatttcccagtgtttgttggatgatacgtttgcctaataagttaaagcttccaaaaatagaaaggaaagcttggtgacactaagctctggcatgctgtcatcttctggctaggggtcttgtgcttctgtttttctatttctgaccaaagacataaaatgtgttgtgacctctcaggtaatgctgtggtatcatgtccaagcagtaactaaccagagtggagtttttacaggtaaaaaagtattgaatagctcagcaacagggaggacttaatgataaattgctttttcagaaaatgacaaggggacagacacttgcggttcttccattgagagccttaaggaagttaagcgaagcctctgctcaccccttcaagtaacgatgacccatcagatcgtggtcataaccagtagcatctgaggcacccctaagctctctgggccccagattccttgttaagttaaatgaggtgacttcccagtttaccattctaggggttcctgaggagttaaaaatgaaggagaaaatgcaacatgtaccaagtgagcaagagctcaaggcttatctctggcacgaaagtgaccgctgtccgttagagatgtgattgccctagatttcctttaggaaatgcaaacattgaactctggccttcaggacccaagtcccggcttgcatctattctatacactttctgttatttgttagggctcacaaggaacagttatttatcttttattttctattttttttttagagatggagtctcactctgtctcccaggctggagtgcagtggtgccatctcagctcactgcaacctccacctcccgggttcaagcaattctcctgtctcagcctcctgagtagctgggactacaggcgtgtgccatcatgcccggctaatttttagtatttttagtagaggcagggtttcaccatattggccaggctggtcttgaactcctg

SEQ ID NO: 94 - DNA, 3708 bp, Homo sapiens
  source 1..3708: mol_type = other DNA
gcctcagagaataaataggagactaagaggacaggcagacaaacaggacgagcctgggggctccaggcctggacggaggaagaagccagctatacggggtttctccatgctggccaagctggtctccaactcctgacctcgtgatccgcctgccttggcctcccaaagtgctggaatttacaggcgtgagccaccacgcccggcctggtcattctttctctccacaatccaatgaggaaactcatctgcccacccaactaccatcataaagtcctcaggtgatttctcaagctatacctccattgggacatctctctccaacctgagaccactggatatcacccacaatgctatatagcacctcaaactcagcatatctatgatgggtgtctgctcagcatccactcgccttttctggtgcactcctcccagcccagctccagaatggccctgccccccagccccctggccatggaatatgtcaatgactttgacttgatgaagtttgaggtaaagcgggaaccctctgagggccgacctggcccccctacagcctcactgggctccacaccttacagctcagtgcctccttcacccaccttcagtgaaccaggcatggtgggggcaaccgagggcacccggccaggcctggaggagctgtactggctggctaccctgcagcagcagctgggggctggggaggcattggggctgagtcctgaagaggccatggagctgctgcagggtcagggcccagtccctgttgatgggccccatggctactacccagggagcccagaggagacaggagcccagcacgtccagctggcagagcggttttccgacgcggcgctggtctcgatgtctgtgcgggagctaaaccggcagctgcggggctgcgggcgcgacgaggcgctgcggctgaagcagaggcgccgcacgctgaagaaccgcggctacgcgcaggcctgtcgctccaagcggctgcagcagcggcgcgggctggaggccgagcgcgcccgcctggccgcccagctggacgcgctgcgggccgaggtggcccgcctggcccgggagcgcgatctctacaaggctcgctgtgaccggctaacctcgagcggccccgggtccggggacccctcccacctcttcctctgagccgttcagagcaccttgtggtgtagtgggggctgggtggggtggctccgcccaggaggcggctgcacggttctctgcatcgttaccagagcgccttctggtcctagccacgccctgtatgaccgcgcaaatatccccaaagcttttgggtcctcaagtcatgcccgaatttagatgctggtcattttctggagaggggtcccctccccttacgaacacagaaacccagcccacatgactagcacgctgagctctgcagggaccagtgccaggcactggggggtggaagtgtggtgacacagtgaatgggaggtggaggagggttgcagctcccacctcagtttagtttttaattcagggttttcaacctgtaacacattaaagctgtaattagcaatgaggctgtattttcattctgaagcttgtaacctccccattttagcactacagaattttcaagatttcaatatccaacaactagatagattaggacctctatccgagatgctttttccctgcccaaccctgtggccttcagggctcagagcagcaaaggcctgaagagtgagctctgggggttgttggtgtgggttgggagagagctgtgtgcagaagtctggaaacctgggtcctagtcccagctcttccatgggatccccctgtcaccctgagcaaatcagttgcttcctggacttgtgttacttcatctaattctcatgtggattggacgacttctgctccctttccagttctggcatctccccagtatggaagtcccggtggtctccccaagaagtccccaagacaatctcgccaaaggcacctcctatcctgctgcagtttcccagctgcagcctaggcaggggatgcacagcccaggcgaggaagcctggcttctctgtgagcacatacgtgggtcctcggcagctccctccaggctgtctgggcctccagacctgcacagggtgctcctgccacctcccacctctctgagggctgaggtgagacttctcctgggatgacaatttgctgagagagtgcagcttttgtgaattaaacttgaagtccaggcagaattctaatgcaataagctaaatgttcttgcaatttaagaagtgttcattctttatccctgcttcaggctactgtttgtgtgtctgtgtgtgggagagggagagagagggagagagagagagagcaagaaaaagggaagtccccacctggagctgggtggcacatctgttatggagagccaatggaaatggggtggaggggcaggcattggaacagatggatcctccccctctcaccaccaccaccaccaaactcagaagcctcagtggcccagctcggcctgtcagtggctatccaggtctcacagtttccatggtaacccagtcctccagttcagcaggctaaggggagggcagatggggcctctgggcagagcacaggcgttgtgctgggagcagccgcctcacgagttactgtcatctgtcctctaccggacataataaatgggagaagctttcgggcatgggggaagaggctcccatatgtccctcttcctttctaccccctctctccaccccctccccagctgtctcttctcttcccatcacagttcttgctccctctttcccctcctctatcccagctcatccctcctccgcttaggagcctgcaatgggagtcctctcctccatcgctctcagaatttctctcgtctcctctcccttccccaccattggccactggccatcctctctggctctggattctctctctgaggggctcacagaacctttagcttctctttcttcatccccttccacactcattgttgaagggagaggatggggcagggagggtccttccgggagcccagctctttttctctcactctaaatttcacctgtgtctatccttgcctgccctggagctgtaattcagacccactacttttcttgttcacttttctcacctgcatggcacacaaagacagacagaaccatccatagacacctgagcacacaactaggccacatatacagtaccatacctgggtctcaaaatgatactcctgggttacctgagtatacttaaaaaaacaaagttgcacactgtggtgattaagagtagtgctttaggtggggcgcagtggttcctgcctgtaatcccaacactttgggaggccaaggaggaagtttgagaccagcctgggcaacatagcgagaaccctgtcactacaaaaaaataaaataaaataaattcagatgtaatggcatgcacctggagtcccacctacccaggaggctaaggtggaaggataacttgagcccaggagttcggggatgcagtgagccataatcgtgccactgcattccagcctgggtggcagagtgagaccctgtttcaagttgcagttctaccagttttttgaccatgggtgggttaaaatcaagcctcacttgattgtcagaaaaatggtgaaaataatggtaaccacctactgagaataaatgcatttaaataagcacttagcataaa

SEQ ID NO: 95 - DNA, 2880 bp, Homo sapiens
  source 1..2880: mol_type = other DNA
gagccctattcacacctcggccaggctgcggtggccaggactggtttgggaaggcagggccccggtgtgcagccacagccagcagcctcctacctacacaagggtgttcgggagcatctcagggccgaagactttgctgcctgccctgccaggactttgtcctcacccctggcaccatgcagctccaggtgttctggactgggctggaatacacctgccggctcctgggcatcaccactgctgcagtgttgatcggcgtgggcactgagaccttcctccagggccagttcaaaagcctggctttctatctgctgtttacaggagccgctgtctccatatgtgaaggggcctactttgtggctcagctgctggccatctgcttccagtgtcaaccagggtccctggcagacagagtaagggagaaagcccactggctgggctgcttccagaagttcctggcctacctgctgctgtcggtggcctgcttcctccacccggtcctggtctggcacgtgaccatcccaggctccatgctcatcatcaccggcctggcctacttccttctgagcaagcggaagaagaggaaagctgcccccgaggtgctggcctccccagagcagtacacagacccctctagcagcgctgtgagcaccaccggctctggggacacagagcaaacctacactttccatggggccctcaaggaggggcccagctcccttttcatccacatgaagagtatcctgaaggggactaagaagcccagtgccctccagccccccaacaccctgatggagctgagcctggagccagccgactccctggccaagaagaagcaggtgcactttgaagacaacttggtccgcatagtcccctccctcgccgaaggtctggatgatggggacagtgagccagaggagaccacctctgacacgacacccatcattccccctccccaggccccactcttcctgtcatctcttacagccaccggcctgttctgagcgcttgctccagcctggaggacgctcagtgaggggtctacctagctcaatggccctccctggagtttcagggtcttctctggtcagcttttcaaggggtaaccagacacccccacactggctgggcccctgaggccatcaggaggtgtgactggccagcatttctggagaggcctcgagggaggcacaaacaaggctcacgccacctcaagccagcacaagctccttcctcctggccacaggtggggaaacccttatgcttccccaacaagaccatcactgccactgcgcacgaggctgcaggtgccattgactcagcccagaagggacagaggatgtctgcccagcaaaagcctctgggcttttcttactcccaacccagggcacaacactcagtgaggaacctatggtccacccatctctgcaggcccagggaagtgtcagtaacttaagcatctctaaggcagagaaggtggctgtgggtcttgaccaaccaagactgctcagggtcgggaggggaggctgcaggcctctggtgggagcagccgggccgtgggccacattctctgcatgctctagggctggggctgagctgctcaggaaaacaatgccgctctgtgctcactgtgggaaagccaggccagatgagcaaagctcattccagaaacatggctggggacttccagcctccttaccacatggcctttccaagattccaggttgatgatgcaaactgaacgcagtgaatgccagggcccagaaggcaatgtctgctgtgggcaggaccacagaggctgcccaggggtggcacagcacagagaagtgtcagtgccgtgtttaggcacttggtagcagaataggtgataaatataaatgtcttcaaaatgaaaggaagtgaggggggaagaagaggagggaaaatgggaggataggaggaagtgggcaggcaggcaagttctctgccatggggccacggaagtgacagttaacccagaaggaacaaagacatctgaatcagatattcctttgtaaatctgtaaaggatttgcaatataggctttacttctaaaaattgttgtcgggtccatgaggctgttgccaggaagagcacacacaccattcttcatggcaactgcagtgagggagcagtcaggaggaagagtcctgtggcttctcagtccctgtggaggctaacgcaaagctctgtagaacaagccttagagtgcatgctctatggtctaatgagaaacttgtgtttgtgcttgcacacgtttcaatgttccctacaggatattttacaaaggtatgaaaataatggccaaccagaagctagtatcagtttggggcctaaatgcagcaggtgttcctgataagcagataagcagagctcttcctgcagtagcccaggtctcaccaaatgacaaatggctaatcaagacactcctgcaagcccttgatggtgagagctgcagagctgtttcttcagctgccatcaaggcaggacctcaggacctgactgtctacagtcagggaatagtgaacaatatgggatatgggtataggtaggtggtagacaaggatcgtgccccatggctgatgggccgggtgtctcaggcaggccccctgtgcacagaaagagcctaagtgcagagaagccaggcatctttcccctgtcaactgtggaaggggaatgtgatggccacagacaccctcatggcacagacatggccagcctctgacgttcctctgcatcagagtagttctgtcctcgaagccccagtcagcagagctggatgaccagatcgcaagaagcaaaccagaaagtgtgatggcagagatgtcagatcccgtttaataaacacttcagtaaacccaa

SEQ ID NO: 96 - DNA, 3625 bp, Homo sapiens
  source 1..3625: mol_type = other DNA
gtcagctgtctgtctgggtgtctatgcgggcgcagcagtgcacccttccccagcctcgggcgctgcgcagggacagacaaggcattcgcagcgccctgcccgcgctccacgcccgcagccgccagacggcagcgcctgcgtccgtgcccgccccagccggtgcgcgggagccgcgggggcaaaggcgcagtggccagcggaccatctctcgtgccctcgctctctgcgctccggggcagctgagccccggccacccgctctccaagatgaagaagctccagggagctcacctccgcaagcctgtcaccccagacctgctgatgacccccagtgaccagggcgatgtcgacctggatgtggactttgctgcacaccgggggaactggacaggcaagctggacttcctgctgtcctgcattggctactgtgtaggcctggggaatgtctggcgcttcccctatcgagcgtacaccaatggaggaggcgccttcctcgtgccctacttcctcatgctggccatctgtggcatccccctcttcttcctggagctctccctgggccagttctccagcctagggcccctggctgtctggaaaatcagccctctcttcaaaggcgccggcgcagccatgctgctcatcgtgggcttggtggccatctactacaacatgatcatcgcctacgtgctcttctacctcttcgcctccctcaccagcgacctaccctgggagcactgtggcaactggtggaacacagaactctgcctggagcacagagtctccaaggacggcaacggggccctgcccctcaacctcacctgcaccgtcagccccagcgaggagtactggagccgctacgtcctccacatccaaggcagccagggcatcggcagccctggggagatccgctggaacctctgcctctgcctgctgctggcctgggtcatcgtgttcctctgtatcctcaagggtgtgaagtcttcgggcaaggtggtgtatttcacggccacgttcccctacctcatcctgctcatgctgctggtccgcggagtcaccctcccaggggcctggaagggcatccagttctatctcaccccccagttccaccacttgttgtcttccaaggtgtggattgaagctgctcttcagatcttctattccctgggtgtgggcttcggggggctcctcacctttgcctcctacaacacgtttcaccagaacatctatagagacactttcatcgtcactctgggcaacgccatcaccagcatcctggctggctttgccatcttctccgtgctgggctacatgtctcaggagctgggcgtgcctgtggaccaagtagccaaagcaggccctggcctggcctttgtcgtctacccacaggccatgaccatgctgcctctgtcacccttctggtcctttctcttcttcttcatgcttctgactctcggcctagatagccagtttgcttttctggagaccattgtgacagctgtgacagatgagttcccatactacctgcggcccaagaaggcggtgttctcagggctcatctgcgtggccatgtacctgatggggctgatcctcaccactgatgggggcatgtactggctggtccttctggatgactacagcgccagcttcgggctgatggtggtggttatcaccacgtgccttgccgtgacacgggtgtatggcattcagaggttctgccgagacatccacatgatgctgggcttcaagccgggcctctacttcagggcctgctggctgttcctgtccccagccacgctcttggccctcatggtgtatagcatcgtcaagtaccagccctcggagtatggcagttaccgcttcccgccctgggctgagctgctgggcatcctgatgggcctgctgtcctgcctcatgatcccagctggcatgctggtggctgtgcttcgagaagagggctcactctgggagcggctccaacaggccagccggccggccatggactggggaccatcgctggaggagaaccggacgggcatgtatgtggccacgctggctgggagccagtcaccaaagccactgatggtgcacatgcgcaagtacgggggcatcaccagcttcgagaacacggccatcgaggtggaccgtgagattgcagaggaggaggagtcgatgatgtgaggcaggaggcaggcgggcagaaggccctgcccgggacctcacagtcccttcttagaagcctgcaaaggtcagctgtgccctctgggattctgagaggctatgggggggcctgccatagggatgccagtcccccagtgggggtcccttctgcagcctctgcctctcctgaaacctctgacaaccccctacacacacacacaggcatactcagacccactcaaagctgagaatgatccaactcagccctactttgcggatggacatattaaggccaggaggggagggacttgccccaggtcgcatggcagaggggacttgaacccacgcctcctgaccagccagctccctttcccatggggcagccggcaccaccttctcatctctattcagggcctacacccctccctttttggggggagcctgtccccacacaccttagcacaaccagaatcttgagttgggcagggaaggtcagggctacagaggctcttgaggctccgaggccctggggatcaaaggtcaatgagttaggtggggagaaagatccctgggggctcctgtatgcaggactcctgaaggaggtgggagctgagggccctggaggatggggaaggtttgcagagagaggaagaagagggcagggtggggaggagctctgggccagaggatgctgagctgcctgggcagagaccaagagggttggagacactagttttaccaggttacaggagggttcaagacagaaggaaaactaacagagggggagccagggatgatggggtgggcaggggatttgcatgatgaggagtatcattccagatctgtgcagagcggatcggacgggacagggtggagcggggaggcctgtgcaaagtgggaaagagctgccggtggcctgagccagggtgctggggctgcgggtggggaaaggaggccccagcacttcagacaaagaccaccccttcacccactggaccccaagccgcaaaagctggggaggtaggaagggtcactgtgagggcccagggcccctctcttcccatctcccttgtcctggtggccttcagtgtctcttccagttccaggagctgctgggaagctggccttcattgttggtgacagggcaaccatgggactaggaggtggcccatacacatggctggctggtcctgaggccacccaccttgttccaggacatgggcccgggcccggtgtgggggtagacgcagagggcagagggtagcccttggtactgtgggtccctcaaagaggagagtggggtgcagaaatgcctcagcatggctcatgcacacagcattctcccgtggggcagggttccctcatgcaacaggcctccctgtggccagcctctctgcctgtctctcgctgcgtccccccaaccccttgtgttctattggttcattgttaataaatatcaaatgaggtca

SEQ ID NO: 97 - DNA, 1208 bp, Homo sapiens
  source 1..1208: mol_type = other DNA
acgctggggcttcaggtctcctagcaacaagttgtaccatatgaggacggccgctgagccataatggagatatcaatgcctccacctcagatatatgtagaaaaaactctggccattatcaaaccagatattgttgacaaagaggaggagatacaagatattattcttagatccggattcaccattgttcagagaagaaaactacgcctcagccctgagcaatgtagtaacttttatgtggaaaagtatggaaaaatgtttttccccaacttaacagcttacatgagttctggaccacttgtcgccatgatattagctagacataaagccatctcttattggttagaacttttgggaccaaataatagcttagtagcgaaggagacacatccagacagtctgagggcaatttatggcacagatgacctaaggaatgcacttcatgggagtaatgactttgctgctgcggaaagagaaatacgttttatgtttcctgaagtgattgttgagcccattccaattggacaagctgctaaggactatttaaatttacatataatgccaactctgcttgaaggactcacagagctttgtaagcaaaaaccagcagatcctttgatttggctagctgattggctgctgaaaaataatcctaacaaacccaaactttgtcaccatccaattgtagaagaaccttattaaaaaaaaaatcctcgaaagaacaaatcatgaactatcttattataaaaggctgtacttctactgtttgaaaaaattatttctagggtttaagtaactacaagtaaaataaatttatttcttaaaaataagtgttaaaggaataactattctatgaagcagataacattaaggtaaaatataggcttagagtatgattttaacaatgatgtaacactaggtaatggaaaactcatgtaagatgttgctagatgtttttgtttttcttaagtttgagaagtaagacataaagtttagttatacattttcccaagcacatatcctatatctaaatacataattaatgcacagttgagacagtctagtttaaaaggcttaggtccttttgtagtccataagcatggtgatttggtttcatgctcatgtgtcagatatgcttccctcaaaccttgttacagcatcatcacattacctgtttgatgtaaaaaataaaaaatagaaaggtgtaa

SEQ ID NO: 98 - DNA, 985 bp, Homo sapiens
  source 1..985: mol_type = other DNA
acttggccctcgctctcactcgcggtcgcctggccgttgtcattgtccctccgctgtcaccttttcaagccccaggctggctgcttcagaagcccctgaccccatggaggagtgggacgtgccacagatgaagaaagaggtggagagcctcaagtaccagctggccttccagcgggagatggcgtccaagaccatccccgagctgctgaagtggatcgaggacgggatccccaaggaccccttcctgaaccccgacctgatgaagaacaacccatgggtggaaaagggcaaatgcaccatcctgtgagccccgcacccggcccctctcacaccatcctgtgagaccacgcccggccccactcccaccatcttgtaagactgtgcccagccccactcactccatcctgtgagtcccactcccagccccactcccaccatcctgtgagcccatgcccggccccactcacaccaacctgtgagccccactcccggccccactcactccatcttgtaagactatgcccagccccattcactccatcctgtgagtcccactcccagccccactcccaccatcctgtgagccccacttccagccccactcccaccatcctgtgagccccactcccagccccactcccaccatcctgtgagccccactcccggccccactcactccatcctgtgagccccactcccagccccactcacaccaacctgtgagccccactcccggccccactcacaacatcttgtaagactgtgcccggccccattcactccatcctgtgagaccacgcccggccccactcactctatcctgtgagaccacgcctggccccactcccaccatcctgtgagccccactcctggccccactcacaccatcctatgagcccacgcccggccccactcccaccatcctgtgaaccccactccactcgcacgtgattacagtctgtaaaggtgtgactttataaagaca

SEQ ID NO: 99 - DNA, 6105 bp, Homo sapiens
  source 1..6105: mol_type = other DNA
aagagcaggaagccaggcagcgggcaggggaggctgcggggccactcgctggagaggcaaacaggaaggactgccccctgagcgccaggcttcgggcccgggaatcgccgccgccgccgccgcagagctgcagctcggggccgagggtaaggaggcgagccgggagcgggaggcccgggagagctccgcgggtccccgcgcccagtccccagccgcgccccgaccccgccgccccgggcctcggctcgccctccgcacccccccctgcccccccaccgttcgccgctgcaggcggtcggccgccgcgatgaaggcgagctcgggggatcaggggagccccccgtgcttcctgcgcttcccgcggcctgtgcgggtggtaagtggcgccgaggccgagctcaagtgcgtggtcctgggggagccgccgcctgtagtggtgtgggagaagggcgggcagcagctggcggcctcggaacgcctgagcttcccggcggacggcgcggagcacggcctgctgctgaccgccgcactgcccaccgacgcgggggtctacgtgtgccgcgcccgcaacgcggccggcgaggcctacgcggcggccgccgtcaccgtgctggagccgccggcctccgaccccgagctgcagcccgccgagcgcccgctgccatcgccggggtccggggagggcgccccggtcttcctcacggggcctcgatcccagtgggtgctgcggggggcggaggtggtgctgacgtgccgggcggggggcctccccgagcccacactgtactgggagaaggacgggatggccctggacgaagtgtgggacagcagccacttcgcgctccagccgggccgcgccgaggacggccccggcgcgagcctggcactgcgcatcctggcggctcggctgccggattccggcgtctacgtgtgccacgcccgcaacgcgcacggccacgcgcaggcgggggcgctgctccaggtgcaccagccccccgagagcccgcccgcggaccccgacgaggcccccgcgccggtggtggagccgctcaagtgcgcgcctaagaccttctgggtgaacgagggcaagcacgccaagttccgctgctacgtgatgggcaagcccgagcccgagatcgaatggcactgggagggccgcccgctgctcccggaccgccgccgcctcatgtaccgcgaccgcgacggcggcttcgtgctcaaggtgctttactgccaggccaaggatcgtgggctctacgtctgcgccgcgcgcaactcggcgggccagacgctcagtgccgtgcagctgcacgtgaaagagccccgcctccggttcacacggcccctgcaggacgtggagggccgtgagcacgggattgccgtgctggaatgtaaagtacccaactcccgcatccccacggcctggttccgtgaggaccagcggctgctgccctgccgcaagtacgagcagatcgaagagggcactgtccggcgcctcatcatccacaggctgaaggcagacgatgatggtatctacctgtgcgagatgcggggccgggtgcgcaccgtggccaacgtcacagtcaaagggcccatcctgaagcgcctgccccggaagctcgacgtcctggaaggagagaatgctgtgctgctagtggaaactctagaggccggggtcgagggacgctggagccgtgatggggaggagctgccggtcatctgccagagcagctcaggccacatgcatgccctggtccttccaggggtcacccgagaggatgctggcgaggtcacctttagcctgggcaactcccgtaccactacgcttctcagagtaaaatgtgtcaagcacagtcccccaggaccccccatattggcagagatgttcaagggccacaagaacacggtcctgttgacctggaagcctcccgagccagctcccgagaccccattcatctaccggctggagcggcaggaagtgggctctgaagactggattcagtgcttcagcatcgagaaagccggagccgtggaggtgccgggcgactgtgtgccctccgagggtgactaccgcttccgcatctgcacagtcagcggacatggccgtagtccccacgtggtgttccacggttctgctcaccttgtgcccacagctcgcctggtggcaggtctggaggatgtgcaggtatacgacggggaagatgccgtcttctccctcgatctctccaccatcatccagggtacctggttccttaatggggaagagctcaagagtaacgagccggagggccaggtggaacctggggccctgcggtaccgtatagagcagaagggtctgcagcacagactcatcctgcatgccgtcaagcaccaggacagcggtgccctggtcggcttcagctgccccggcgtgcaggactcagctgccctcacaatccaagagagcccggtgcacatcctgagcccccaggacagggtgtcgttgaccttcacaacctcagagcgggtggtgctgacttgtgagctctcaagggtggacttcccggcaacctggtacaaggatgggcagaaggtggaggagagcgagttgctggtggtgaagatggatgggcgcaaacaccgtctgatcctgcctgaggccaaagtccaggacagtggcgagtttgagtgcaggacagaaggggtctcggccttcttcggcgtcactgtccaagatcctcccgtgcacatcgtggacccccgagaacatgtgttcgtgcatgccataacttccgagtgtgtcatgctggcctgtgaggtggaccgagaggacgcccctgtgcgttggtacaaggacgggcaggaggtggaggagagtgacttcgtggtgctggagaatgaggggccccatcgccgcctggtgctgcccgccacccagccctcagacgggggcgagtttcagtgcgtcgctggagatgagtgtgcctacttcactgtcaccatcacagacgtctcctcgtggatcgtgtatcccagcggcaaggtgtatgtggcagccgtgcgcctggagcgtgtggtgctgacctgtgagctatgccggccctgggcagaggtgcgctggaccaaggatggagaggaggtggtggagagccccgcgctgctcctgcagaaggaagacactgtccgccgcctggtgctgcccgctgtccagctcgaggactccggcgagtacttgtgtgaaattgacgatgagtcggcctccttcactgtcaccgtcacagaacccccagtgcggatcatataccctcgcgatgaggtgaccttgatcgccgtgaccttggagtgtgtggtgctgatgtgtgaactgtctcgggaggatgcccctgtgcgctggtacaaggatgggctggaagtggaggagagcgaggccctggtgctggagagggatgggccacgctgccgcctggtgctacctgctgctcagcccgaggacgggggcgagtttgtatgtgatgctggagatgactcggccttcttcactgtcactgtcacagccccaccagagaggattgtgcacccggcagcccgctccctggatctgcattttggggctccagggcgcgtggagctgcgctgtgaggtggccccagctgggtctcaggtgcgctggtacaaggacgggctggaagtggaggcatcagatgccctgcagctgggtgccgaggggcccacccgcaccctgaccctgccccacgcccagcctgaggacgccggggagtatgtgtgtgagacccggcatgaggccatcaccttcaatgtcatcctggctgagcctccagtgcagttccttgctctagagacaactccaagcccgctctgtgtggcccctggggagccagtggtgctgagctgtgaactgtcccgggctggcgcccccgtggtctggagccacaatgggaggcccgtgcaggagggcgagggcctagagctccatgccgagggcccccgccgagtcctctgcatccaggctgcaggcccagcccatgcagggctctacacctgccagtctggagcagcccccggagccccaagcctcagcttcaccgtccaggtggctgagccccctgtgcgggtggtagctcccgaggcagcccagacgagggttcggagcaccccaggcggggacctagagctggtggtgcacctctccgggccagggggccctgtacgctggtacaaggacggggagcgactggcaagccaggggcgggtgcagctggagcaggccggggccaggcaggtgctgcgggtgcagggggcacggagcggggacgctggggagtacctgtgcgatgcgccccaggacagccgcatcttccttgtcagcgtggaagagccactgctggtgaagctggtctcggagctgacaccactcactgtccacgagggcgatgatgccacgttccggtgtgaagtctccccaccagatgccgatgtcacctggctgcgcaatggggccgtcgtcactccagggccccaggtggagatggcccagaatggttcaagccgcatcttaaccttgcgaggctgccaactgggggatgcagggaccgtgactttgcgggcagggagcacggccacaagtgcccggctccatgttcgagagacagagctgctgttcctacggcggttgcaggatgtgcgggcagaggaaggccaggatgtgtgtctcgaagtggagacaggccgagtgggtgcagcgggggccgtgcgctgggtgcgaggtgggcagcccctgccccacgactctcgcctgtccatggcccaggatgggcacatccaccgcctcttcatccatggtgtcatactggccgaccagggcacctacggctgcgagagccaccacgatcgcaccctggccaggctcagcgtgaggccgaggcagctgagggtgctgcggcctctggaggacgtgaccatcagtgaggggggcagtgccaccttccagctggagctgtcccaggaaggtgtgaccggggagtgggcccggggtggagtacagctgtatccaggacccaagtgtcacatccactcggacggccaccgtcaccgactggtactcaatggcctgggcctggccgactcaggctgtgtctccttcacagcggattccctgcgctgcgcagccagactcattgtgagagaggtcccagtgaccatcgtgcgggggccacacgacctagaggtgaccgagggcgacacagctacgttcgagtgcgagctttcccaagctttggctgatgttacctgggagaaggacgggaacgcgcttacgcctagcccgcggctccggctccaggccctcggcacgcgccgccttctccagctgcgacgctgcggcccctcggacgccgggacctacagctgcgcggtggggacggcccgcgccggaccggtccgcctgaccgtgcgcgagcgtactgtggcggtactctccgagctgcggtcggtgagcgcccgcgaaggcgacggcgctacgttcgagtgcaccgtgtcggaggtcgagaccacggggcgctgggagctcggaggccgcccgctgagacccggagcccgcgtccgcatccgacaggaagggaagaaacacattctggtgcttagcgagctgcgcgccgaggacgccggtgaagtccgcttccaggcggggcccgcccagtccctggctctactggaagtggaggcattgcctctccagatgtgccgccacccccctcgcgagaagaccgttctggtgggccgccgggcggtgctggaggtgactgtgtcccgctcggggggccacgtgtgctggctgcgggagggggccgagctgtgcccgggagataagtatgagatgcgcagccacggccccacccacagcctggtcatccatgacgttcgacctgaggaccaaggcacttactgctgccaggccggccaggacagcacccacacacggctgctggtagagggcaactaggagaacctaaccaggccaggcgggtgcccttggacagcttggaaggcgtttgcccttaccctgggcaggggtagagagacaaggaacaataaaagtgctacagctca

SEQ ID NO: 100 - DNA, 1575 bp, Homo sapiens
  source 1..1575: mol_type = other DNA
gtcctgtggcctctgcagctcagcatggctagggtactgggagcacccgttgcactggggttgtggagcctatgctggtctctggccattgccacccctcttcctccgactagtgcccatgggaatgttgctgaaggcgagaccaagccagacccagacgtgactgaacgctgctcagatggctggagctttgatgctaccaccctggatgacaatggaaccatgctgttttttaaaggggagtttgtgtggaagagtcacaaatgggaccgggagttaatctcagagagatggaagaatttccccagccctgtggatgctgcattccgtcaaggtcacaacagtgtctttctgatcaagggggacaaagtctgggtataccctcctgaaaagaaggagaaaggatacccaaagttgctccaagatgaatttcctggaatcccatccccactggatgcagctgtggaatgtcaccgtggagaatgtcaagctgaaggcgtcctcttcttccaaggtgaccgcgagtggttctgggacttggctacgggaaccatgaaggagcgttcctggccagctgttgggaactgctcctctgccctgagatggctgggccgctactactgcttccagggtaaccaattcctgcgcttcgaccctgtcaggggagaggtgcctcccaggtacccgcgggatgtccgagactacttcatgccctgccctggcagaggccatggacacaggaatgggactggccatgggaacagtacccaccatggccctgagtatatgcgctgtagcccacatctagtcttgtctgcactgacgtctgacaaccatggtgccacctatgccttcagtgggacccactactggcgtctggacaccagccgggatggctggcatagctggcccattgctcatcagtggccccagggtccttcagcagtggatgctgccttttcctgggaagaaaaactctatctggtccagggcacccaggtatatgtcttcctgacaaagggaggctataccctagtaagcggttatccgaagcggctggagaaggaagtcgggacccctcatgggattatcctggactctgtggatgcggcctttatctgccctgggtcttctcggctccatatcatggcaggacggcggctgtggtggctggacctgaagtcaggagcccaagccacgtggacagagcttccttggccccatgagaaggtagacggagccttgtgtatggaaaagtcccttggccctaactcatgttccgccaatggtcccggcttgtacctcatccatggtcccaatttgtactgctacagtgatgtggagaaactgaatgcagccaaggcccttccgcaaccccagaatgtgaccagtctcctgggctgcactcactgaggggccttctgacatgagtctggcctggccccacctcctagttcctcataataaagacagattgcttcttcgcttctcactgaggggccttctgacatgagtctggcctggccccacctccccagtttctcataataaagacagattgcttcttcacttgaa

SEQ ID NO: 101 - DNA, 2035 bp, Homo sapiens
  source 1..2035: mol_type = other DNA
ggaagtccgtagtgtctcattgcagataatttttagcttagggcctggtggctaggtcggttctctcctttccagtcggagacctctgccgcaaacatgctccgccagatcatcggtcaggccaagaagcatccgagcttgatccccctctttgtatttattggaactggagctactggagcaacactgtatctcttgcgtctggcattgttcaatccagatgtttgttgggacagaaataacccagagccctggaacaaactgggtcccaatgatcaatacaagttctactcagtgaatgtggattacagcaagctgaagaaggaacgtccagatttctaaatgaaatgtttcactataacgctgctttagaatgaaggtcttccagaagccacatccgcacaattttccacttaaccaggaaatatttctcctctaaatgcatgaaatcatgttggagatctctattgtaatctctattggagattacaatgattaaatcaataaataactgaaacttgatatgtgtcacttttttatgctgaaagtatgctctgaactttagagtataggaaattaactattagaatttaaagaatttcttgaatttctgtagtttgaaaatacgactttaagctgctttagtaaaacacttccattttgtgtatagactgttggtaacttcactagagcatacataacaactggaactggaaattatacaaaagtaaattgggaaggatactccagcatctgacactggcaaaatggaaacctttgagtttctcttactggctgttgaagtgtgtgcagtttttaacaatggtttttacttggcatctctttgttgtgattttcaaggttataagttgctttggtcctaggattgaagttgaaatctgagtttatcagtgctaaccatggtgctagtagtcaagagatcttgagaattttggctgctgagtcttggtgcagggtgcaggttttcttttcttttttcttttttttttttttgagatagtctctgtcacccaggctggagtgcagtggtacaaacatggatcactgcagcctctacctcccgggcttaagtgatcctcctgcctcagcccctaagtagccgggactacaggtatgtgccaccatgcccagttaatttttgtaattttttttagagacagggttttgccatgttgcccaggctggtctcaaactcttgagctcaagcgatccattctcctcagcctcccagggtgctgggattacaggcgtgagccattgcgcttagccatggtgcaggttttcaaaggccaggaagtatattcataattttaagatggggaatatagcaagttttcacataggtgtgtgtaagtcatcacatcatagaaacttgaggaattcagtgacattaattttggattttcatacgtaagtatacaattaaatgtttacagggtagtagaagcacattttaaatgtcaggaactgaactaagtatttgaattacgtggattatctcaaaaattttgaaattgttaaacgagttgaattacttgaattcattctgttagtcaaatggtggatatttacacccatgtagttttgaatttagagtgtgtagagtgttttcagttaccagactccatgcttttacctcctatgtgtcaggtataatttgaacctctaagaacagggtttctcaaccttgccactgttgactatttctgaaagacagtttggtttagcagaccatcccatgcgctttagcttgtttagtagctaacttgggctctgccactacagacaaaaagcactctttccctccaattcccacaggctatgagaagaatggagacattaccaaatgtccattggtgggcaaaattgcttcattcctacctctgttgagaattactctagatcctttggcacaaattacctcaaagtttaaaattgtgtaaacaaacagtgtgtcatgtaattgaaaaacattaagcaactccaaataaatgctacattaagaaattagtaa

SEQ ID NO: 102 - DNA, 2694 bp, Homo sapiens
  source 1..2694: mol_type = other DNA
gagagcccagagagctgaacctgcatcccggacctgcggcgaccgtcgtacaccatgggcctccacctccgcccctaccgtgtggggctgctcccggatggcctcctgttcctcttgctgctgctaatgctgctcgcggacccagcgctcccggccggacgtcaccccccagtggtgctggtccctggtgatttgggtaaccaactggaagccaagctggacaagccgacagtggtgcactacctctgctccaagaagaccgaaagctacttcacaatctggctgaacctggaactgctgctgcctgtcatcattgactgctggattgacaatatcaggctggtttacaacaaaacatccagggccacccagtttcctgatggtgtggatgtacgtgtccctggctttgggaagaccttctcactggagttcctggaccccagcaaaagcagcgtgggttcctatttccacaccatggtggagagccttgtgggctggggctacacacggggtgaggatgtccgaggggctccctatgactggcgccgagccccaaatgaaaacgggccctacttcctggccctccgcgagatgatcgaggagatgtaccagctgtatgggggccccgtggtgctggttgcccacagtatgggcaacatgtacacgctctactttctgcagcggcagccgcaggcctggaaggacaagtatatccgggccttcgtgtcactgggtgcgccctgggggggcgtggccaagaccctgcgcgtcctggcttcaggagacaacaaccggatcccagtcatcgggcccctgaagatccgggagcagcagcggtcagctgtctccaccagctggctgctgccctacaactacacatggtcacctgagaaggtgttcgtgcagacacccacaatcaactacacactgcgggactaccgcaagttcttccaggacatcggctttgaagatggctggctcatgcggcaggacacagaagggctggtggaagccacgatgccacctggcgtgcagctgcactgcctctatggtactggcgtccccacaccagactccttctactatgagagcttccctgaccgtgaccctaaaatctgctttggtgacggcgatggtactgtgaacttgaagagtgccctgcagtgccaggcctggcagagccgccaggagcaccaagtgttgctgcaggagctgccaggcagcgagcacatcgagatgctggccaacgccaccaccctggcctatctgaaacgtgtgctccttgggccctgactcctgtgccacaggactcctgtggctcggccgtggacctgctgttggcctctggggctgtcatggcccacgcgttttgcaaagtttgtgactcaccattcaaggccccgagtcttggactgtgaagcatctgccatggggaagtgctgtttgttatcctttctctgtggcagtgaagaaggaagaaatgagagtctagactcaagggacactggatggcaagaatgctgctgatggtggaactgctgtgaccttaggactggctccacagggtggactggctgggccctggtcccagtccctgcctggggccatgtgtcccccctattcctgtgggcttttcatacttgcctactgggccctggccccgcagccttcctatgagggatgttactgggctgtggtcctgtacccagaggtcccagggatcggctcctggcccctcgggtgacccttcccacacaccagccacagataggcctgccactggtcatgggtagctagagctgctggcttccctgtggcttagctggtggccagcctgactggcttcctgggcgagcctagtagctcctgcaggcaggggcagtttgttgcgttcttcgtggttcccaggccctgggacatctcactccactcctacctcccttaccaccaggagcattcaagctctggattgggcagcagatgtgcccccagtcccgcaggctgtgttccaggggccctgatttcctcggatgtgctattggccccaggactgaagctgcctcccttcaccctgggactgtggttccaaggatgagagcaggggttggagccatggccttctgggaacctatggagaaagggaatccaaggaagcagccaaggctgctcgcagcttccctgagctgcacctcttgctaaccccaccatcacactgccaccctgccctagggtctcactagtaccaagtgggtcagcacagggctgaggatggggctcctatccaccctggccagcacccagcttagtgctgggactagcccagaaacttgaatgggaccctgagagagccaggggtcccctgaggcccccctaggggctttctgtctgccccagggtgctccatggatctccctgtggcagcaggcatggagagtcagggctgccttcatggcagtaggctctaagtgggtgactggccacaggccgagaaaagggtacagcctctaggtggggttcccaaagacgccttcacgctggactgagctgctctcccacagggtttctgtgcagctggattttctctgttgcatacatgcctggcatctgtctccccttgttcctgagtggccccacatggggctctgagcaggctgtatctggattctggcaataaaagtactctggatgctgtaa

SEQ ID NO: 103 - DNA, 2434 bp, Homo sapiens
  source 1..2434: mol_type = other DNA
aaggtggaagctgggtccggctgccaggaagcggccggtgcgcgggctgcgcgggctgcgcggggctcggcttccacgtgcgagcctgcaccgccgccccgccctctcctggcggccaggcctggggactctctgccgcgggacggcgcctcgcgttccgccgtggcagtggccgtggccagaacgtccacgcgcggtcgtccaccgccggatctcgctccagcgcccgtggtccagcgtgtagggagccgatcgcccatggagggtctgggccgctcgtgcctgtggctgcgtcgggagctgtcgcccccgcggccgcggctcctgctcctggactgccgcagccgcgagctgtacgagtcggcgcgcatcggtggggcgctgagcgtggccctgccggcgctcctgctgcgccgcctgcggaggggcagcctgtcggtgcgcgcgctcctgcctgggccgccgctgcagccgcccccgcctgcccccgtgctcctgtacgaccagggcgggggccggcgccggcgcggggaggccgaggccgaggccgaggagtgggaggccgagtcggtgctgggcaccctgctgcagaagctgcgagaggaaggctacctggcctactacctccagggaggcttcagcagattccaggccgagtgccctcacctgtgtgagaccagccttgctggccgtgccggctccagcatggcgccggtgcccggtccagtgcccgtggtggggttgggcagcctgtgcctgggctccgactgctctgatgcggaatccgaggctgaccgcgactccatgagctgtggcctggattcggagggtgccacacccccaccagtggggctgcgggcatccttccctgtccagatcctgcccaacctctatctgggcagtgcccgggattccgccaatttggagagcctggccaaactgggcatccgctacatcctcaatgtcacccccaacctcccaaacttcttcgagaagaatggtgactttcactacaagcagatccccatctccgaccactggagccagaacctgtcgcggttctttccggaggccattgagttcattgatgaggccttgtcccagaactgcggggtgctcgtccactgcttggcgggggtcagccgttctgtcaccgtcactgtggcctacctcatgcagaagctccacctctctctcaacgatgcctatgacctggtcaagaggaagaagtctaacatctcccccaacttcaacttcatggggcagttgctggactttgagcgcagcttgcggctggaggagcgccactcgcaggagcagggcagtggggggcaggcatctgcggcctccaacccgccctccttcttcaccacccccaccagtgatggcgccttcgagctggcccccacctagggccccgtggccggcaggccggcccctgccccacccccacccacgggtgtccctgcccactcgtgtggcaagggaggggagggcaggagggctcggcctgagcagggtgctggggggagagcgcaatacctcacgcgggctgccgtcctaatcaacgtgcctatggcgggaccacgctcggagcctgcctcttctgcgactgttactttttctttgcgggatgggggtgggggttccctctccaggtggttgtccaggcccaggtcccggccctgggtgctcagccagctcggctaggccctgcgcctccctgcgcttcccccttcaggaagggtgtgtgccacctcgttgcactggatcccagtggctgcttgggggagaggcgtttgccatcactggtgttgtcacctccctgtttctccaccaagggcttgggcctctcggggctggggcctcccaggggatggggacccagagtgcagtggccgcccacatccatggcctaggagctactgggcaggttcccggccacacatctggtgggctgttttgttttttttttttcctcttcccccagatgtcttgacgggatcactggggctctttgtgagtgagggtggccaaactaccgccggaggagatggggtctcagagcgagagctgcggagggggaggggaagaagaaggcctcacttttgctgctgcggggcccacacagccgctgctactttggggggtggggaaggggccaagctgcagacacacacagtcattcatttctgtccacacccctgtgggtggcgggtgtgcgtgtgtgtgcttgtgtgtgcgcacgtgtcggcgctcacacacacatgctagcccactgatgcacccagcccagggctggcagtctttgcagcgtggggccgtctcaccctggagcctggagaggatctatgcttgtttgtttttgtaatccatatcatagttgctttctttaattgttccttctgaataaacagtttatttaagatactga

SEQ ID NO: 104 - DNA, 2893 bp, Homo sapiens
  source 1..2893: mol_type = other DNA
ctttaccactactcgctatagagccctggtcaagttctctccacctctctatctatgtctcagtttcttcatctgtaacatcaaatgaataataataccaatctcctagacttcataagaggattaacaaagacaaaatatgggaaaaacataacatggcgtcccataattattagatcttattattgacactaaaatggcattaaaattaccaaaaggaagacagcatctgtttcctctttggtcctgagctggttaaaaggaacactggttgcctgaacagtcacacttgcaaccatgatgcctaaacattgctttctaggcttcctcatcagtttcttccttactggtgtagcaggaactcagtcaacgcatgagtctctgaagcctcagagggtacaatttcagtcccgaaattttcacaacattttgcaatggcagcctgggagggcacttactggcaacagcagtgtctattttgtgcagtacaaaatcatgttctcatgcagcatgaaaagctctcaccagaagccaagtggatgctggcagcacatttcttgtaacttcccaggctgcagaacattggctaaatatggacagagacaatggaaaaataaagaagactgttggggtactcaagaactctcttgtgaccttaccagtgaaacctcagacatacaggaaccttattacgggagggtgagggcggcctcggctgggagctactcagaatggagcatgacgccgcggttcactccctggtgggaaacaaaaatagatcctccagtcatgaatataacccaagtcaatggctctttgttggtaattctccatgctccaaatttaccatatagataccaaaaggaaaaaaatgtatctatagaagattactatgaactactataccgagtttttataattaacaattcactagaaaaggagcaaaaggtttatgaaggggctcacagagcggttgaaattgaagctctaacaccacactccagctactgtgtagtggctgaaatatatcagcccatgttagacagaagaagtcagagaagtgaagagagatgtgtggaaattccatgacttgtggaatttggcattcagcaatgtggaaattctaaagctccctgagaacaggatgactcgtgtttgaaggatcttatttaaaattgtttttgtattttcttaaagcaatattcactgttacaccttggggacttctttgtttatccattcttttatcctttatatttcatttgtaaactatatttgaacgacattccccccgaaaaattgaaatgtaaagatgaggcagagaataaagtgttctatgaaattcagaactttatttctgaatgtaacatccctaataacaaccttcattcttctaatacagcaaaataaaaatttaacaaccaaggaatagtatttaagaaaatgttgaaataatttttttaaaatagcattacagactgaggcggtcctgaagcaatggtttttcactctcttattgagccaattaaattgacattgctttgacaatttaaaacttctataaaggtgaatatttttcatacatttctattttatatgaatatactttttatatatttattattattaaatatttctacttaatgaatcaaaattttgttttaaagtctactttatgtaaataagaacaggttttggggaaaaaaatcttatgatttctggattgatatctgaattaaaactatcaacaacaaggaagtctgctctgtacaattgtccctcatttaaaagatatattaagcttttcttttctgtttgtttttgttttgtttagtttttaatcctgtcttagaagaacttatctttattctcaaaattaaatgtaatttttttagtgacaaagaagaaaggaaacctcattactcaatccttctggccaagagtgtcttgcttgtggcgccttcctcatctctatataggaggatcccatgaatgatggtttattgggaactgctggggtcgaccccatacagagaactcagcttgaagctggaagcacacagtgggtagcaggagaaggaccggtgttggtaggtgcctacagagactatagagctagacaaagccctccaaactggcccctcctgctcactgcctctcctgagtagaaatctggtgacctaaggctcagtgtggtcaacagaaagctgccttcttcacttgaggctaagtcttcatatatgtttaaggttgtctttctagtgaggagatacatatcagagaacatttgtacaattccccatgaaaattgctccaaagttgataacaatatagtcggtgcttctagttatatgcaagtactcagtgataaatggattaaaaaatattcagaaatgtattggggggtggaggagaataagaggcagagcaagagctagagaattggtttccttgcttccctgtatgctcagaaaacattgatttgagcatagacgcagagactgaaaaaaaaatttactttgatctctgtttttgaattcttattatttatattttgcttactaccttttttgccttttgtccttttgtggagagggcgatgaattaagagtagtggggtgggtggcgagtaagtaaaatttcaaatagcattttagtgaatgcaattcagtagtcctgaagcctgacttaataaaacaaaattcatttatggttgtttcatgaaagttattagataggatcaagttttgtttgtatattgtttgccttgctctgtttttgcttttgtgcttaggcaagtgcaaaatactctatggaaatcataattgtcatcttcttcatggtgttagggatatattgtcacatgatttcaataaatcctttttttgcttta

SEQ ID NO: 105 - DNA, 3470 bp, Homo sapiens
  source 1..3470: mol_type = other DNA
actgggtcaagcacagccctgagcggccgcgtgtccgaggcccaggtgccctctagagccctgtagttcctgcccctctctgcccctctcggctcctgctgttccgccgctgtcgtccgaaccatcccaacccccagtccacccagacagcgcccgagctaggggagggaacggtctgggagtcggcagctggcgccagggcggccggaggatgccgaggggccggagccgggagggcccgaggccgaggcgcactctacccccagctcctaccctgtaagccccgccagcctccggacgtgctgtccctgggcccgtcgccctcggggctcccgccggaactccttcactctcagaggccgagtccctcccctccccacggctgcgtgtggccgttgccgtctgcacccagaccctgagccgccgccgccggccatggaggtggcgcccgagcagccgcgctggatggcgcacccggccgtgctgaatgcgcagcaccccgactcacaccacccgggcctggcgcacaactacatggaacccgcgcagctgctgcctccagacgaggtggacgtcttcttcaatcacctcgactcgcagggcaacccctactatgccaaccccgctcacgcgcgggcgcgcgtctcctacagccccgcgcacgcccgcctgaccggaggccagatgtgccgcccacacttgttgcacagcccgggtttgccctggctggacgggggcaaagcagccctctctgccgctgcggcccaccaccacaacccctggaccgtgagccccttctccaagacgccactgcacccctcagctgctggaggccctggaggcccactctctgtgtacccaggggctgggggtgggagcgggggaggcagcgggagctcagtggcctccctcacccctacagcagcccactctggctcccaccttttcggcttcccacccacgccacccaaagaagtgtctcctgaccctagcaccacgggggctgcgtctccagcctcatcttccgcggggggtagtgcagcccgaggagaggacaaggacggcgtcaagtaccaggtgtcactgacggagagcatgaagatggaaagtggcagtcccctgcgcccaggcctagctactatgggcacccagcctgctacacaccaccccatccccacctacccctcctatgtgccggcggctgcccacgactacagcagcggactcttccaccccggaggcttcctggggggaccggcctccagcttcacccctaagcagcgcagcaaggctcgttcctgttcagaaggccgggagtgtgtcaactgtggggccacagccacccctctctggcggcgggacggcaccggccactacctgtgcaatgcctgtggcctctaccacaagatgaatgggcagaaccgaccactcatcaagcccaagcgaagactgtcggccgccagaagagccggcacctgttgtgcaaattgtcagacgacaaccaccaccttatggcgccgaaacgccaacggggaccctgtctgcaacgcctgtggcctctactacaagctgcacaatgttaacaggccactgaccatgaagaaggaagggatccagactcggaaccggaagatgtccaacaagtccaagaagagcaagaaaggggcggagtgcttcgaggagctgtcaaagtgcatgcaggagaagtcatcccccttcagtgcagctgccctggctggacacatggcacctgtgggccacctcccgcccttcagccactccggacacatcctgcccactccgacgcccatccacccctcctccagcctctccttcggccacccccacccgtccagcatggtgaccgccatgggctagggaacagatggacgtcgaggaccgggcactcccgggatgggtggaccaaacccttagcagcccagcatttcccgaaggccgacaccactcctgccagcccggctcggcccagcaccccctctcctggagggcgcccagcagcctgccagcagttactgtgaatgttccccaccgctgagaggctgcctccgcacctgaccgctgcccaggtggggtttcctgcatggacagttgtttggagaacaacaaggacaactttatgtagagaaaaggaggggacgggacagacgaaggcaaccatttttagaaggaaaaaggattaggcaaaaataatttattttgctcttgtttctaacaaggacttggagacttggtggtctgagctgtcccaagtcctccggttcttcctcgggattggcgggtccacttgccagggctctgggggcagatttgtggggacctcagcctgcaccctcttctcctctggcttccctctctgaaatagccgaactccaggctgggctgagccaaagccagagtggccacggcccagggagggtgagctggtgcctgctttgacgggccaggccctggagggcagagacaatcacgggcggtcctgcacagattcccaggccagggctgggtcacaggaaggaaacaacattttcttgaaaggggaaacgtctcccagatcgctcccttggctttgaggccgaagctgctgtgactgtgtccccttactgagcgcaagccacagcctgtcttgtcaggtggaccctgtaaatacatcctttttctgctaacccttcaaccccctcgcctcctactctgagacaaaagaaaaaatattaaaaaaatgcataggcttaactcgctgatgagttaattgttttatttttaaactctttttgggtccagttgattgtacgtagccacaggagccctgctatgaaaggaataaaacctacacacaaggttggagctttgcaattctttttggaaaagagctgggatcccacagccctagtatgaaagctgggggtggggaggggcctttgctgcccttggtttctgggggctggttggcatttgctggcctggcagggggtgaaggcaggagttgggggcaggtcaggaccaggacccagggagaggctgtgtccctgctggggtctcaggtccagctttactgtggctgtctggatccttcccaaggtacagctgtatataaacgtgtcccgagcttagattctgtatgcggtgacggcggggtgtggtggcctgtgaggggcccctggcccaggaggaggattgtgctgatgtagtgaccaagtgcaatatgggcgggcagtcgctgcagggagcaccacggccagaagtaacttattttgtactagtgtccgcataagaaaaagaatcggcagtattttctgtttttatgttttatttggcttgttttattttggattagtgaactaagttattgttaattatgtacaacatttatatattgtctgtaaaaaatgtatgctatcctcttattcctttaaagtgagtactgttaagaataataaaatactttttgtgaa

SEQ ID NO: 106 - DNA, 3260 bp, Homo sapiens
  source 1..3260: mol_type = other DNA
gtagccggagtgagaccgctctgcaaaccactgcgtgctttgcagagtgattatcagcacagttccctgccctggataaggaacagctacagtcgctgttaaatgtgcctgaaaagcaatttgcaatctttgcattaggcatttcggccgtggaaccccaggctcggaggactgggtgtgagcgctgcccgggagaggctgacctgccgggaccggagtgcccggggacgctgtgcccccacttgcccaacgtgcggaatcggctaagcgcgtcggcctgcgcggggcacaagggacgacgcccgcctttctctctccgagaaggatccccaaacctcactctcttcactcctccccgctaaaaaaaaaaaaaaaaagaaaagggtaaaaaaatccccctccccctcccggccgcggcagcctctgcctggcaggcgcgtgctccgaggtttcgccggggtactgcgcggctccgtgacccacccgccgacggcggttcgggccccgacccgggcgctgcggagtctcgaacgcccgtgacgtggatttttcgctcgtcaggaagccgccatcccttccttcgttccttgcgaggagcgactagaacccgagttctctgagcacaccgtgccgtcagctggcccgtcacgcactcgactccgccactcccctacttgtttttctgagacttgggaagccttcctgaaaggatttgtaaaaactggttcttggaaaggggctgcagaagacctcccgaggtggatgttactgagccgcccggcgagcaccagccgtgacaggtccgggcccggccgcgcggagccgcgggagaactgccctccgcagccggaagggggccgcggggagcgctcctggccggtggcggcgggggcgggggaggccgcgagccgaggcgctccaactccggcgatgccgcctcgccctccccgccgcggttgatgcggcacgtgcacgccgccgcccgcaccaggacctgggccgggtcgcacggcttggcccccggccaccgcttcggcctaggatttcagatgcatgccaggtttccactgattgccagaactcgagatcactacacatggatccccaaaatcaacatggcagtggcagttcgttagttgtgatccagcagccttctttggatagccgtcagagattagactatgagagagagattcagcctactgctattttgtccttagaccagatcaaggccataagaggcagcaatgaatacacagaagggccttcggtggtgaaaagacctgctcctcggacagcaccaagacaagaaaagcatgaaaggactcatgaaatcataccaattaatgtgaataataactacgagcacagacacacaagccacctgggacatgcagtactcccaagtaatgccaggggccccattttgagcagatcaaccagcactggaagtgcagccagctctgggagcaacagcagtgcctcttctgaacagggactgttaggaaggtcaccaccaaccagaccagtccctggtcataggtctgaaagggcaatccggacccagcccaagcaactgattgtggatgacttgaagggttccttgaaagaggacctgacacagcacaagttcatttgtgaacagtgtgggaagtgcaagtgtggagaatgcactgctcccaggaccctaccatcctgtttggcctgtaaccggcagtgcctttgctctgctgagagcatggtggaatatggaacctgcatgtgcttagtcaagggcatcttctaccactgctccaatgacgacgaaggggattcctattcagataatccttgctcctgttcacaatcacactgctgctctagatacctgtgtatgggagccatgtctttatttttaccttgcttactctgttatcctcctgctaaaggatgcctgaagctgtgcaggaggtgttatgactggatccatcgcccagggtgcagatgtaagaactccaacactgtctattgtaagctggagagctgcccctcccggggtcagggtaaaccatcatgatttttggaggtgggttgtacctcctgaacttttagctttcaagttgtggctgttttttgtttttgtttttgtttttgttttctttagaatttttccctgtttcccaccttctcttcccctgttgccaaggtctaactcatggatttttctctttcctcatggatgatcttcagcaagagtggactgggaagctgcacctggctcccactttcaacaagagcctctgccatccacttgagggtattgagagccagtgggcttttgtgtagcctttttgttctgcaagcaactttctaaagttgtgtacatgaacatacacccacatccagactacagtgatttagagttgttttgattgggtaccgtgggagcagggaaattggttttttaaaaagcaactgtttaattgcttaaataagctatgtattaaatctgtctccagttagggctatcttcctagcataggccccttaagtagcatgggggatatattttttgctataacgtaaaaattttcctttaaccactgccctctccttctttctccttcaaggttctttccccctcagttttgttgttgtcttactctggagatgccaagtgtattttttctttctatgtaattttagattcgccttacaatgtaaatcttcacattggagataatattggttggaccttgcccatcttcactctagccttcgtatttgtgaaggactcagccaccttccttcttcaccccatgcttctcaccaaatttttgttgtcattgagggcacttggataactcaagttgatatttatagctgatcaatctatatgtgtcacagaactatgctgcctaaagtgatcttggctccttaatggtccttttggccccttggatagttaacagctgagtaattctaatctcttctgtgttttccttgccttaaccacaaattgtggtgctttttgtatattttatgtataaatcacaaagttgaattctgactatttttaagacaaaagtctgttaaacttttttattgtaaagaatatttattatgcgaatctctattattttatggtatttattgcaaaagactgttgaaatgtactcatgtttgaatataacaaaatatcaatacttaacggaaaataaggtgacacgaagaaagtacatatgttaactataatgcagaaaatatattaattaatgaaa

SEQ ID NO: 107 - DNA, 5018 bp, Homo sapiens
  source 1..5018: mol_type = other DNA
ctagaaacatagttgatgttcttacccttatcatataaggataagattatccttatcatccttatcatcatgataaggaaaacagctaggaagtttaaccataggtcaaattctcatgcatagggagaaagggttctgctaacacttttcctatactgacccttgtggaaataacttctaactgaatgagctcattcattgattgtgctagccaaccttatcacatcttaattatagcacttcctcctacagagaacccaacaatttggtatagtacattctagatagagtttgttactatctaattctgggattcctgctcttttggccacatttatctgttatggcttactctatatcatttattctatttaaatgtgatgttagtaatatttcaacttcaccactttaatgcagccatctatggataagtaattggaaatctgtgctataattttccttttagtcatagtcttcctggctcattctgtttgcctgcttacctgatgtgggcatgcctctatcagtgcctgttagttttgaataaaaagcttctgtaagcacctgggcaccaggtcagagaatttctccaggctaggcttcctgtcatctgtcttaaagggcctcatgaatgtattcccctcttctggacctgattattattaaatcttagctcctttttcttgtctgtttctatttggacttggcagtaggactttgtgctgtaccttgatgctactttggtaacatctgtggctgcatccaggattgcaccttggtttcattcagctgctagagaagataagagatgccatactcttacattcacctatgggcctccataatatcatttgccagggatattttgccatttcccatctgagcaagtacagagaaatccagcaatgagatgcctagctgtttggctcacaaaactacaactaatgcattactgtgtatctcattccagggtacctggattgacagttaaatgcctaagagaaaatagccacatatgatctaaacataaacccagatttataaaggaaacacctacaagggctgacttaattttttctaaggaaaaccagaacatagcaaatggttatggcacaaatctatgtatgtagatagagaaggcaaagcaaagggcatcagaaagaatgggattaattgagaaaggttttctcaaggaggtattgggtctttgaacttgatctattagatcactgtaaatcgctataggtgaatcactgaggttgaaagagtggcattggcatggccaattggtgaaacttgttgtggcagcaacagattagaggggagagagaaattggattctggtcatccatgaatatgtggctaggaggtgggtgggtaggagtgaggaattaggatttccaccatattctggcatttttatgaagaaaaactttaccgtaaaacattgacagaaacattgctggactcaagctctctggtttggattctaagtttttggagcctgctgtgactcttccctctcgccctcctgctcttggaggagtggactcccagcatcacctaggcttgctgtaattaggcatttctaaggagaacaggatactaatgaagaaatagtaatgtaatccttggaagatttgcatctcagtaaaatcaggtggcccttgatcatgaatggctcatttgccatgctgggaaaaattaaaaaggagatgtccttcctggctggatactggtgtctgcttatacattttggtatttcttctgcctccactatcagcaccacaactgctgaatcctcaatgagtaaagatgcttcggttattctatttctctgctattattgcctcagttattttaaattttgtaggaatcattatgaatctgtttattacagtggtcaattgcaaaacttgggtcaaaagccatagaatctcctcttctgataggattctgttcagcctgggcatcaccaggtttcttatgctgggactatttctggtgaacaccatctacttcgtctcttcaaatacggaaaggtcagtctacctgtctgctttttttgtgttgtgtttcatgtttttggactcgagcagtgtctggtttgtgaccttgctcaatatcttgtactgtgtgaagattactaacttccaacactcagtgtttctcctgctgaagcggaatatctccccaaagatccccaggctgctgctggcctgtgtgctgatttctgctttcaccacttgcctgtacatcacgcttagccaggcatcaccttttcctgaacttgtgactacgagaaataacacatcatttaatatcagtgagggcatcttgtctttagtggtttctttggtcttgagctcatctctccagttcatcattaatgtgacttctgcttccttgctaatacactccttgaggagacatatacagaagatgcagaaaaatgccactggtttctggaatccccagacggaagctcatgtaggtgctatgaagctgatggtctatttcctcatcctctacattccatattcagttgctaccctggtccagtatctccccttttatgcagggatggatatggggaccaaatccatttgtctgatttttgccaccctttactctccaggacattctgttctcattattatcacacatcctaaactgaaaacaacagcaaagaagattctttgtttcaaaaaatagtggaatttcagtaaacaatacctagatttacctgatggtttgggggcaagattttctgtttgcagttttcccagtgatctggggaattcagttttgtgattgctgatctgacatcataggcttttgagtgcctgaatttcagttcattctgtaattttttttttttggtatgtaagtattttaaaataaggcacattctgtaagaaacttttttgaggcattatttgtcatgtattttgcatggccattgaatccatgtataatggaaatcatcacattgtttcattgtctattgaagtataatgggaaattcttccaaattagagaacacattggggtgcacgtgatgagttagatatttgtggtttagctgaggagccctgagtaagggctcagcagtggataggtggtcactgggagccactggactgaccgaaagcagaggaagggggttcagtgcaaagagaaaaggaaaaaaggaaggtttcagagagaaaagaaggaaagagaaaggtgaagagaaataggcgtacactgggccaggcgcggtggctcatgcttgtaatcccagctctttgggaggccgaggcaggtgaatcgtgaggtcaggagatcgagaccatcctggctaacacggtgaaaccctgtctctactaaaaatacaaaaaaattagctgggcgtggtggcgggcacctgtagtcccagctactcgggaggctgaggcaggagaatggcttcaacccaggaggcggagcttgcagtgagatgagatcgcgccactgcactccagtctgggcgacagagcaagactctgtctcaaaaaaagaaatagacgtacactctgggacaagacagaaaaagtatcctcacacttgctgggtaatagtaactgtgctattcctagcaagaaaccctaaggtttctaggcttgttctggaaaagaggatattgccagagttggtaagtagaggaaagtgattagtatttaagaaactgaatcaagaatggttccaaaaatcttaaaatccagggaaaccagcttaaggtcaaaggcaagtgctagataatgaaattaacaccatatgagtacaaatttacaaaatactgtacttcaccataggctatggaactcagtggaaagccaaaaaagcactggacaggtggtgggagtgcttgacttgtggtgaatttatttcatctgcttatacatttgtacacagcacagttcttctccagaatgttgtttctgtggctgggaccattggactgtggctccaaaagaacaacagtctgaagatgggatgttgacaagttttggaatgtcaattggggaatcataatttgtatttatttaggttttttttggtgttcataaaatgcttatgtaatatttacatatgtcaaataaatcttaaaattttataaattacatgacttttctcattctggccaccagaatcttgactgcaatattcagtaaaaattacccaagccaatattttgtgctatgaaacctggaggtattcatcatgttaaatcctaagagagataaaaggattactgagagtgattaaatgcatactatgtcttaagaactctaaatgagtggcagaaaccctcattggtccctcagtatctgttctttctcctctcttccatagtaatcaaatcattagctgttcagaattttggcagcaaattccattattgccatgtatgtgagcagaaaagatgtatataatttctgggtggtacttcggagcagtcctaccttcccttctttaatcttttctttgttactccctccatgagctggaatgctgacatcatgattgtctatcttggacaatgcagatgaggtgaatgcttgggatagtgaagtaattctccatcctagagctggctccatggttcttcacactgtggacttgtaacaccagctctggactgctcaagcctgaactccaggactgttgatgagaaatcagtgtctccattctttcaggtgctggagttttggtctctgttaccttagtcaaataacctatgctataccttaaatatacacaataatatatatctttatgatgaaatagtatgtatttaccatcttattagtcaaagaacctttttatatgacctactaagtgaaattgaaataaaacctgtacatgtggtataataccattttttttggtaaaaatgtgtacatatgaagaaaagtctaaaaggaaatatataaaaatgtaaacagtaattattttaggtgggaggaatcaataaatttttttgtgtatttcaa

SEQ ID NO: 108 - DNA, 1683 bp, Homo sapiens
  source 1..1683: mol_type = other DNA
ggtcatttgaagactctctcggaagagatagcgtcttgctgcaacctgcggtcccagcagaaaaaccttgtgatccttgttgcgggcgacatggaagacgactcactctatttgggaggtgactggcagttcaatcacttttcaaaactcacatcttctcggctagatgcagcttttgctgaaatccagcggacttctctctctgaaaagtcaccactctcatctgagacccgtttcgacctctgtgatgatttggctcctgtggcaagacagcttgctcccagggagaagcttcctctgagtagcaggagacctgctgcggtgggggctgggctccagaagataggaaataccttctatgtgaacgtttccctgcagtgcctgacatacacactgccgctttccaactacatgctgtcccgggaggactctcaaacgtgtcatcttcacaagtgctgcatgttctgtactatgcaagctcacatcacatgggccctccacagtcctggccatgtcatccagccctcacaggtattggctgctggcttccatagaggtgagcaggaggatgcccatgaatttctcatgtttactgtggatgccatgaaaaaggcatgccttcccgggcacaagcagctagatcatcactccaaggacaccaccctcatccaccaaatatttggagcgtattggagatctcaaatcaagtatctccactgccacggcgtttcagacacctttgacccttacctggacatcgccctggatatccaggcagctcagagtgtcaagcaagctttggaacagttggtgaagcccaaagaactcaatggagagaatgcctatcattgtggtctttgtctccagaaggcgcctgcctccaagacgttaactttacccacttctgccaaggtcctcattcttgtattgaagagattctccgatgtcacaggcaacaaacttgccaagaatgtgcaatatcctaagtgccgtgacatgcagccatacatgtctcagcagaacacaggacctcttgtctatgtcctctatgctgtgctggtccacgctgggtggagttgtcacaacggacattacttctcttatgtcaaagctcaagaaggccagtggtataaaatggatgatgccgaggtcactgcctctggcatcacctctgtcctgagtcaacaggcctatgtcctcttttacatccagaagagtgaatgggaaagacacagtgagagtgtgtcaagaggcagggaaccaagagcccttggtgctgaagacacagacaggccagcaacgcaaggagagctcaagagagaccacccttgcctccaggtacccgagttggacgagcacttggtggaaagagccactcaggaaagcaccttagaccactggaaattcccccaagagcaaaacaaaacgaagcctgagttcaacgtcagaaaagttgaaggtaccctgcctcccaacgtacttgtgattcatcaatcaaaatacaagtgtggtatgaaaaaccatcatcctgaacagcaaagctccctgctaaacctctcttcgacgaaaccgacagatcaggagtccatgaacactggcacactcgcttctctgcaagggagcaccaggagatccaaagggaataacaaacacagcaagagatctctgcttgtgtgccagtga

SEQ ID NO: 109 - DNA, 2283 bp, Homo sapiens
  source 1..2283: mol_type = other DNA
cttctacctgtgcggccctcaacgtctccttggtgcgggacccgcttcactttcggctcccggagtctccctccactgctcagacctctggacctgacaggagacgcctacttggctctgacgcggcgccccagcccggctgtgtccccggcgccccggaccaccctccctgccggctttgggtgcgttgtggggtcccgaggattcgcgaggtaaggccgccctcgcgttgtttggtctcaggctgcagaccaggtcaggcgactcttgcagtttctccagcttgcctggtgcgtgctggtgtctcgccgcttggtcctctcctgttcttcggcccccggttgccagattgcttttcttccaggcctcaaccagatttgttgaaagacattcaagattacgaagtttagatgaccaaaatggatatccgaggtgctgtggatgctgctgtccccaccaatattattgctgccaaggctgcagaagttcgtgcaaacaaagtcaactggcaatcctatcttcagggacagatgatttctgctgaagattgtgagtttattcagaggtttgaaatgaaacgaagccctgaagagaagcaagagatgcttcaaactgaaggcagccagtgtgctaaaacatttataaatctgatgactcatatctgcaaagaacagaccgttcagtatatactaactatggtggatgatatgctgcaggaaaatcatcagcgtgttagcattttctttgactatgcaagatgtagcaagaacactgcgtggccctactttctgccaatgttgaatcgccaggatcccttcactgttcatatggcagcaagaattattgccaagttagcagcttggggaaaagaactgatggaaggcagtgacttaaattactatttcaattggataaaaactcagctgagttcacagaaactgcgtggtagcggtgttgctgttgaaacaggaacagtctcttcaagtgatagttcgcagtatgtgcagtgcgtggccgggtgtttgcagctgatgctccgggtcaatgagtaccgctttgcttgggtggaagcagatggggtaaattgcataatgggagtgttgagtaacaagtgtggctttcagctccagtatcaaatgattttttcaatatggctcctggcattcagtcctcaaatgtgtgaacacctgcggcgctataatatcattccagttctgtctgatatccttcaggagtctgtcaaagagaaagtaacaagaatcattcttgcagcatttcgtaactttttagaaaaatcaactgaaagagaaactcgccaagaatatgccctggctatgattcagtgcaaagttctgaaacagttggagaacttggaacagcagaagtacgatgatgaagatatcagcgaagatatcaaatttcttttggaaaaacttggagagagtgtccaggaccttagttcatttgatgaatacagttcagaacttaaatctggaaggttggaatggagtcctgtgcacaaatctgagaaattttggagagagaatgctgtgaggttaaatgagaagaattatgaactcttgaaaatcttgacaaaacttttggaagtgtcagatgatccccaagtcttagctgttgctgctcacgatgttggagaatatgtgcggcattatccacgaggcaaacgggtcatcgagcagctcggtgggaagcagctggtcatgaaccacatgcatcatgaagaccagcaggtccgctataatgctctgctggccgtgcagaagctcatggtgcacaactgggaataccttggcaagcagctccagtccgagcagccccagaccgctgccgcccgaagctaagcctgcctctggccttcccctccgcctcaatgcagaaccagtagtgggagcactgtgtttagagttaagagtgaacactgtttgattttacttggaatttcctctgttatatagcttttcccaatgctaatttccaaacaacaacaacaaaataacatgtttgcctgttaagttgtataaaagtaggtgattctgtatttaaagaaaatattactgttacatatactgcttgcaatttctgtatttattgttctctggaaataaatatagttattaaaggattctcactccaaacatggcctctctctttacttggactttgaacaaaagtcaactgttgtctcttttcaaaccaaattgggagaattgttgcaaagtagtgaatggcaaataaatgttttaaaatctatcgctctatcaa

SEQ ID NO: 110 - DNA, 7401 bp, Homo sapiens
  source 1..7401: mol_type = other DNA
agttgccctagttagcgcggagagggcgagcatccgcggagggggtgcgagctgcgcggccgcctgcccggcgtggggagcgggcgccccgggccgccgccgagagggggagccggaagtcggcgcgggcgcccctcggccccgacgacgccgtgaccttggccggcccgctcacctctcggaatctcgcctgccgggaccgcgaaatgggggcgaccgcggcgtccctctccgtcggcgcagccctcgacggcgcttgggtttgaccccggcgcgcttgctcgccccttctcccgagaaggggtttcggagatctatccatactaatggaaatgtaactggaactgactctgatgataaaatcaaggaccatcaagcaagatcatgcagtaggcaactttgcttccaaaagaagttaccaacatttagaatttctacttattctgaggttccagttacagctatatcagagaatgagttaatctcctcagaaatcactaaatactactctgaggggcttagaaattaacaggttgtttatataattggccttaaatgaggtgagagtgaagagactagagccatctctggaaaacatcattatcccattccccgggaagctaccctctggaactcaagatttgaccatatctgttttgaggattcattatgaacaaagaagtctcccaggtgtgaagtttttcaacatgagtggcctcggggacagttcatccgaccctgctaacccagactcacataagaggaaaggatcgccatgtgacacactggcatcaagcacggaaaagaggcgcagggagcaagaaaataaatatttagaagaactagctgagttactgtctgccaacattagtgacattgacagcttgagtgtaaaaccagacaaatgcaagattttgaagaaaacagtcgatcagatacagctaatgaagagaatggaacaagagaaatcaacaactgatgacgatgtacagaaatcagacatctcatcaagtagtcaaggagtgatagaaaaggaatccttgggacctcttcttttggaggctttggatggatttttctttgttgtgaactgtgaagggagaattgtatttgtgtcagagaatgtaaccagctacttaggttacaatcaggaggaattaatgaatacgagcgtctacagcatactgcacgtgggggatcatgcagaatttgtgaagaatctgctaccaaaatcactagtaaatggagttccttggcctcaagaggcaacacgacgaaatagccatacctttaactgcaggatgctaattcaccctccagatgagccagggaccgagaaccaagaagcttgccagcgttatgaagtaatgcagtgtttcactgtgtcacagccaaaatcaattcaagaggatggagaagatttccagtcatgtctgatttgtattgcacggcgattacctcggcctccagctattacgggtgtagaatcctttatgaccaagcaagatactacaggtaaaatcatctctattgatactagttccctgagagctgctggcagaactggttgggaagatttagtgaggaagtgcatttatgcttttttccaacctcagggcagagaaccatcttatgccagacagctgttccaagaagtgatgactcgtggcactgcctccagcccctcctatagattcatattgaatgatgggacaatgcttagcgcccacaccaagtgtaaactttgctaccctcaaagtccagacatgcaacctttcatcatgggaattcatatcatcgacagggagcacagtgggctttctcctcaagatgacactaattctggaatgtcaattccccgagtaaatccctcggtcaatcctagtatctctccagctcatggtgtggctcgttcatccacattgccaccatccaacagcaacatggtatccaccagaataaaccgccagcagagctcagaccttcatagcagcagtcatagtaattctagcaacagccaaggaagtttcggatgctcacccggaagtcagattgtagccaatgttgccttaaaccaaggacaggccagttcacagagcagtaatccctctttaaacctcaataattctcctatggaaggtacaggaatatccctagcacagttcatgtctccaaggagacaggttacttctggattggcaacaaggcccaggatgccaaacaattcctttcctcctaatatttcgacattaagctctcccgttggcatgacaagtagtgcctgtaataataataaccgatcttattcaaacatcccagtaacatctttacagggtatgaatgaaggacccaataactccgttggcttctctgccagttctccagtcctcaggcagatgagctcacagaattcacctagcagattaaatatacaaccagcaaaagctgagtccaaagataacaaagagattgcctcaattttaaatgaaatgattcaatctgacaacagctctagtgatggcaaacctctggattcagggcttctgcataacaatgacagactttcagatggagacagtaaatactctcaaaccagtcacaaactagtgcagcttttgacaacaactgccgaacagcagttacggcatgctgatatagacacaagctgcaaagatgtcctgtcttgcacaggcacttccaactctgcctctgctaactcttcaggaggttcttgtccctcttctcatagctcattgacagaacggcataaaattctacaccggctcttacaggagggtagcccctcagatatcaccactttgtctgtcgagcctgataaaaaggacagtgcatctacttctgtgtcagtgactggacaggtacaaggaaactccagtataaaactagaactggatgcttcaaagaaaaaagaatcaaaagaccatcagctcctacgctatcttttagataaagatgagaaagatttaagatcaactccaaacctgagcctggatgatgtaaaggtgaaagtggaaaagaaagaacagatggatccatgtaatacaaacccaaccccaatgaccaaacccactcctgaggaaataaaactggaggcccagagccagtttacagctgaccttgaccagtttgatcagttactgcccacgctggagaaggcagcacagttgccaggcttatgtgagacagacaggatggatggtgcggtcaccagtgtaaccatcaaatcggagatcctgccagcttcacttcagtccgccactgccagacccacttccaggctaaatagattacctgagctggaattggaagcaattgataaccaatttggacaaccaggaacaggcgatcagattccatggacaaataatacagtgacagctataaatcagagtaaatcagaagaccagtgtattagctcacaattagatgagcttctctgtccacccacaacagtagaagggagaaatgatgagaaggctcttcttgaacagctggtatccttccttagtggcaaagatgaaactgagctagctgaactagacagagctctgggaattgacaaacttgttcaggggggtggattagatgtattatcagagagatttccaccacaacaagcaacgccacctttgatcatggaagaaagacccaacctttattcccagccttactcttctccttctcctactgccaatctccctagccctttccaaggcatggtcaggcaaaaaccttcactggggacgatgcctgttcaagtaacacctccccgaggtgctttttcacctggcatgggcatgcagcccaggcaaactctaaacagacctccggctgcacctaaccagcttcgacttcaactacagcagcgattacagggacaacagcagttgatacaccaaaatcggcaagctatcttaaaccagtttgcagcaactgctcctgttggcatcaatatgagatcaggcatgcaacagcaaattacacctcagccacccctgaatgctcaaatgttggcacaacgtcagcgggaactgtacagtcaacagcaccgacagaggcagctaatacagcagcaaagagccatgcttatgaggcagcaaagctttgggaacaacctccctccctcatctggactaccagttcaaatggggaacccccgtcttcctcagggtgctccacagcaattcccctatccaccaaactatggtacaaatccaggaaccccacctgcttctaccagcccgttttcacaactagcagcaaatcctgaagcatccttggccaaccgcaacagcatggtgagcagaggcatgacaggaaacataggaggacagtttggcactggaatcaatcctcagatgcagcagaatgtcttccagtatccaggagcaggaatggttccccaaggtgaggccaactttgctccatctctaagccctgggagctccatggtgccgatgccaatccctcctcctcagagttctcttctccagcaaactccacctgcctccgggtatcagtcaccagacatgaaggcctggcagcaaggagcgataggaaacaacaatgtgttcagtcaagctgtccagaaccagcccacgcctgcacagccaggagtatacaacaacatgagcatcaccgtttccatggcaggtggaaatacgaatgttcagaacatgaacccaatgatggcccagatgcagatgagctctttgcagatgccaggaatgaacactgtgtgccctgagcagataaatgatcccgcactgagacacacaggcctctactgcaaccagctctcatccactgaccttctcaaaacagaagcagatggaacccaggacaagaagacagaagagttcttctctgtggtgactacagactagaggaatgctctacaggtgcaacaggttcaggtgtttgctgacgtccagtgtacagtgaatctggtaggcggggacccttacctgaaccagcctggtccactgggaactcaaaagcccacgtcaggaccacagaccccccaggcccagcagaagagcctccttcagcagctactgactgaataaccacttttaaaggaatgtgaaatttaaataatagacatacagagatatacaaatatattatatatttttctgagatttttgatatctcaatctgcagccattcttcaggtcgtagcatttggagcaaaaaaaaaaaaaaaaaaaaaaaaaggagtttgcttttgtcgggagattgaaagatgtttttgtttctttctttgtaaaggccttggatattgaaaaaataccaaggcagaacagttggacaatctatttcttgagccaaatttaattattcttatttttgtaatcagtcattggcttcttatctggatgaaggcttttggaggagaaccaaaacgacaagttccaagaagaagatgaagctccgcctccgccgcttagtcccaaccctgcccaggaagaagggcccgtggggctttgcctgtgcccgtccaccaaaggctgtcatgtgtctcgaaatcagcagccctccccatcccaatcccaggcagcttgtgtgtacaatcagcttctctagcaactctgtatctgttggcttcaagagaatattttgcctccacatatgtaccccttctcctttttttaaagatggatttaaaccaagatgcctccaggaaagaggacgaaatgagtatattcacagaggaatccaaaaaatacagtttgggggaaaatgcaataatttttgatgagatgggtgaaggacaagaagtgagttgtgtcaattattgtagatacaattttctgattaaatctggaaaaataaaaggcagcctgttttttctgcttttattgtattaacagctgaggtagctaaagttatttaaaataaaattaaatttatgatccaagtagcttatttttccctttaaatctcattgtaaatatatttgatttcttgtagaaattgatttccttctgtttaattttatgcttttattatactcttgatttttctaaatttgtgtgtgaaatataacattgattgaattgcagttacatttggttagtaatatttcattattttaataactgtgatgtcatgtatggatttactttggggttcaaatcaaaatgtcactgccagaaagagctgttccagctgatctagagcatactgccctagagtgtccctgggatcatctgaacagaagtgcacaggctacttgtacagagaaaaaattaatactcaaaggaaatcttcattttttagattgactttgggaatttgaattttcatcagtgcaaatataaatttctctatcctgctctgaggctaattggtaccatattttccctttgtgtcttgtgactctgccacatcccatctcatcctggcctctgagtcaagaacccagtgaactgactttctagttctagaagttccgctgcaaggccaggaaagcttgagaaaggtattgtggaagaagcaaaggtagacccccatcactcacctttgtctgcatccctgggcctgtgaatgatgacagcacctgacattctgcaccagctacctctgcctccatggcagagaaaaggccataagaacagtggaagaggagcatggactcagacttcaaggaagaagccatttccccaggtccttccttctgcatctcaccacccctagttacaaataactccattgaacagcatctattcagaaactatgccgaataaaaagattggtggaagggctcatgtggttagcaactatgaaacagaaataggacactcagttacaaacattatctcctttagtttttcagaaaatgcatccctgatttcattcatttccagcttgaaagccagccatattactctagtccctaccaaactgctctagaaggtcatttccattttgtttgtgatatttagacgcgcagacttcaggaagttcacctttaacttcagcattccagatgaagtttcctgactcagtgcttttgcataaggaactagaaaaaaaaagtaaggaaaattggagatgctaacatcctcccccatcccaactgcaccttaaaatatgcatgtcaccttcaaggttttataattgcactgtttgttttatgtatgtacagattaaaattattgctacatttgaggaaaataaaattgcttgcttctatgtaattcctgtcattccacaggaaattcacttttccagctactgaatagaatttgtttaacaacctcatgggttgcctcttcatttacagggaagaaatgaaatgtacatctgcagaaattgccaaagcccaaattaacgagtattataaaaggataataaagcatccctgaagccaaa

(19) United States
(12) Patent Application Publication
(10) Pub. No.: US 2024/0240185 A1
(43) Pub. Date: Jul. 18, 2024

(54) HOST FACTORS THAT ENHANCE VIRAL PRODUCTION VIA VIRALLY DRIVEN FITNESS-BASED CRISPR SCREENING

(71) Applicant: Wisconsin Alumni Research Foundation, Madison, WI (US)

(72) Inventors: Andrew Mehle, Madison, WI (US); Cason King

(21) Appl. No.: 18/516,385

(22) Filed: Nov. 21, 2023

Related U.S. Application Data
(60) Provisional application No. 63/384,541, filed on Nov. 21, 2022.

(51) Int. Cl. C12N 15/113; C12N 7/00; C12N 9/22; C12N 15/11

(52) U.S. Cl. CPC C12N 15/1131; C12N 7/00; C12N 9/22; C12N 15/11; C12N 2310/14; C12N 2310/20; C12N 2320/10; C12N 2760/16151

(57) ABSTRACT

Described herein are compositions and methods for a screening approach for identifying host factors that impact influenza viral production after the initial infection. Host factors that enhance influenza virus production were identified. Screening methods described herein include variations of the Clustered Regularly Interspaced Short Palindromic Repeats (CRISPR)/Cas9 system, termed CRISPR activation (CRISPRa) and CRISPR inhibition (CRISPRi).

### CROSS REFERENCE TO RELATED APPLICATIONS

This application is a continuation of U.S. Provisional Application Ser. No. 63/384,541, filed Nov. 21, 2022, the contents of which are specifically incorporated herein by reference in its entirety.

### STATEMENT OF GOVERNMENT SUPPORT

This invention was made with government support under AI125897 awarded by the National Institutes of Health awarded by the National Institutes of Health. The government has certain rights in the invention.

### INCORPORATION BY REFERENCE OF SEQUENCE LISTING

This application contains a Sequence Listing which has been submitted electronically in ST26 format and is hereby incorporated by reference in its entirety. Said ST26 file, created on Mar. 18, 2024, is named “800132US1.xml” and is 323,493 bytes in size.

### BACKGROUND

Viruses are completely dependent upon the host for replication. Like all viruses, influenza virus exploits cellular processes to support its replication while simultaneously evading antiviral responses deployed by the cell in an attempt to block the infection. The balance between these pro- and anti-pathogen forces influences the outcome of an infection, the severity of disease, and even the potential to establish a pandemic outbreak.

Influenza virus is a serious public health threat causing annual epidemics and occasional pandemics with significant morbidity and mortality. Identifying cellular genes and proteins required by influenza virus is essential to understanding the viral life cycle and establishing a mechanistic foundation for the development of host-directed anti-viral therapeutics. Most genetic approaches to identify host factors regulating infection have relied upon loss-of-function screens, which only probe those genes already expressed in the system under study and are limited in their ability to detect contributions from genes essential for cell viability, genes with redundant functions, or gene products needed in limited quantities. Such studies leave a large amount of genetic space unexplored and raises the possibility that entirely new classes of viral co-factors have yet to be discovered.

### SUMMARY

Employing a screening approach for identifying host factors that impact influenza viral production after the initial infection, host factors that enhance influenza virus production were identified. Those factors are useful to study the regulation of the expression of viral genes and replication of the viral genome. Screening described herein include variations of the Clustered Regularly Interspaced Short Palindromic Repeats (CRISPR)/Cas9 system, termed CRISPR activation (CRISPRa) and CRISPR inhibition (CRISPRi). In those methods, the sequence of a single guide RNA (“sgRNA”) directs Cas9 to a specific location, and the catalytically inactive Cas9 has been modified to recruit transcriptional activators or repressors to modify gene expression at that location.

An influenza virus was used to express the CRISPR sgRNA, in a technique referred to as transcriptional regulation by pathogen-programmed Cas9 (TRPPC). This way, the construct is inactive until after a virus infects a host cell and begins to be transcribed, and only the Cas9-expressing and influenza-infected cells are affected. To thoroughly blanket the genome, a library of 70,000 sequences (about 3 targeting sequences for each human gene) was prepared, which incorporated the sgRNA sequences into the influenza genome in between the two coding regions of the influenza NS gene segment and ensured proper cleavage via insertion of a microRNA sequence. In embodiments, the M gene segment may be employed. The library was used to perform a genetic selection by allowing all viruses to compete with each other through multiple rounds of replication in human lung cells. Viruses that activated pro-viral host factors gained a replicative advantage and came to quickly dominate the viral population, and those viruses and their host gene targets were easily determined by deep sequencing. This process can be adapted to any pathogen capable of delivering the targeting RNA.

In embodiments, a nucleic acid vector comprises a heterologous promoter operably linked to an open reading frame encoding a polypeptide having at least 80% amino acid sequence identity to one of SEQ ID Nos. 1-36 or 74-91 or a portion thereof with the activity of SEQ ID Nos. 1-36 or 74-91. In embodiments, the promoter is a viral promoter. In embodiments the promoter is a CMV promotes, retroviral LTRs (e g., HIV, MLV), or an adenovirus promoter like E1A. In embodiments, the polypeptide has at least 90% or 95% amino acid sequence identity to one of SEQ ID Nos. 1-36 or 74-91 or the portion thereof. In embodiments, the vector is a viral vector. In embodiments, the vector is a plasmid.

Further provided is a host cell having the vector or the genome of which is augmented with nucleic acid encoding a polypeptide having at least 80% amino acid sequence identity to one of SEQ ID Nos. 1-36 or 74-91 or a portion thereof with the activity of SEQ ID Nos. 1-36 or 74-91 or comprising a polypeptide having at least 80% amino acid sequence identity to one of SEQ ID Nos. 1-36 or 74-91 or a portion thereof with the activity of SEQ ID Nos. 1-36 or 74-91. In embodiments, the host cells can comprise eukaryotic cells. In embodiments, the host cells can comprise prokaryotic cells. The vector or nucleic acid can be maintained extrachromosomally or stably integrated into the genome of the host cell. In embodiments, the host cell can comprise an insect cell, a plant cell, or a mammalian cell. In embodiments, the host cell is a MDCK cell or derivatives thereof, MDBK, VERO, A549, 293T, CaLu3, MRC5, avian eggs such as chicken eggs. In embodiments, the host cell comprises transgenic eggs expressing a polypeptide having at least 80% amino acid sequence identity to one of SEQ ID Nos. 1-36 or 74-91 or a portion thereof with the activity of SEQ ID Nos. 1-36 or 74-91.

Also provided is method to increase influenza virus yield in cells, comprising: contacting influenza virus and cells comprising the vector comprising a nucleic acid encoding a polypeptide having at least 80% amino acid sequence identity to one of SEQ ID Nos 1-36 or 74-91 or a portion thereof with the activity of SEQ ID Nos. 1-36 or 74-91 or contacted with a polypeptide having at least 80% amino acid sequence identity to one of SEQ ID Nos. 1-36 or a portion thereof with the activity of SEQ ID Nos. 1-36 or 74-91; and collecting progeny influenza virus. The cells can be human, canine, or non-human primate cells. In embodiments, the cells are Vero cells, MDCK cells, 293T or PER C6® cells, or MvLu1 cells. The cells can be contacted with the vector or the polypeptide before contacting the cells with the influenza virus. In embodiments, the cell is contacted with the vector or the polypeptide after contacting the cells with the influenza virus. The yield of influenza virus in cells contacted with the vector or the polypeptide can be increased at least two-fold relative to the corresponding yield in host cells not contacted with the vector or the polypeptide.

In embodiments, a method to detect influenza virus in a sample is provided, comprising: contacting cells having the vector comprising a nucleic acid encoding a polypeptide having at least 80% amino acid sequence identity to one of SEQ ID Nos. 1-36 or 74-91 or a portion thereof with the activity of SEQ ID Nos. 1-36 or 74-91 and a biological sample; and determining whether the sample comprises influenza virus. In embodiments, the cells are human, canine or non-human primate cells. In embodiments, the cells are Vero cells, MDCK cells, 293T or PER.C6® cells, or MvLu1 cells. In embodiments, the sample is a physiological sample. In embodiments, the sample is a nasal sample. In embodiments, the sample is a physiological fluid sample. In embodiments, the method does not include employing nucleic acid amplification.

A method to decrease influenza virus replication in a mammal is provided, comprising: administering to the mammal a composition that inhibits or prevents expression of a polypeptide having at least 80% amino acid sequence identity to one of SEQ ID Nos 1-36 or 74-91 or a portion thereof with the activity of SEQ ID Nos. 1-36 or 74-91.

Further provided is a method to screen for compounds that alter the activity of a pathogen, comprising contacting cells expressing a polypeptide having at least 80% amino acid sequence identity to one of SEQ ID Nos. 1-36 or 74-91 or a portion thereof with the activity of SEQ ID Nos. 1-36 or 74-91 or an isolated polypeptide having at least 80% amino acid sequence identity to one of SEQ ID Nos. 1-36 or 74-91 or a portion thereof with the activity of SEQ ID Nos. 1-36 or 74-91 and a sample having a pathogen; and determining whether the polypeptide alters the activity of the pathogen. In embodiments, the pathogen is a virus. In embodiments, the cells are mammalian cells. For example, the cells can be canine, non-human primate, or human cells. In embodiments, the cells are MDCK cells. Any cell, e.g., any avian or mammalian cell, such as a human, e.g., 293T or PER.C6® cells, or canine, e.g., MDCK, bovine, equine, feline, swine, ovine, rodent, for instance mink, e.g., MvLu1 cells, or hamster, e.g., CHO cells, or non-human primate, e.g., Vero cells, including mutant cells, which supports efficient replication of influenza virus can be employed.

In embodiments, a method to inhibit expression of pro-viral genes in a mammal is provided, comprising administering to the mammal an effective amount a composition that specifically inhibits the expression of a polypeptide having at least 80% amino acid sequence identity to one of SEQ ID Nos. 1-36 or 74-91. In embodiments, the composition comprises RNA. In embodiments, the RNA comprises RNAi. In embodiments, the RNA comprises siRNA. In embodiments, the amount prevents or inhibits influenza virus replication.

In embodiments, a method to screen for inhibitory compounds is provided, comprising combining cells expressing a polypeptide having at least 80% amino acid sequence identity to one of SEQ ID Nos. 1-36 or 74-91 or a portion thereof with the activity of SEQ ID Nos. 1-36 or 74-91 or isolated nucleic acid that encodes a polypeptide having at least 80% amino acid sequence identity to one of SEQ ID Nos. 1-36 or 74-91 or a portion thereof with the activity of SEQ ID Nos. 1-36 or 74-91 and one or more test compounds; and determining whether the one or more test compounds inhibit expression of the polypeptide or inhibit transcription or translation of the isolated nucleic acid. Any cell, e.g., any avian or mammalian cell, such as a human, e.g., 293T or PER.C6® cells, or canine, e.g., MDCK, bovine, equine, feline, swine, ovine, rodent, for instance mink, e.g., MvLu1 cells, or hamster, e.g., CHO cells, or non-human primate, e.g., Vero cells, including mutant cells, which supports efficient replication of influenza virus can be employed.

In addition, disclosed herein are methods to prevent, inhibit, or treat influenza virus infection in an avian or a mammal is provided, comprising administering to the avian or mammal an effective amount of RNA that triggers RNA interference (RNAi), wherein the RNA encodes a polypeptide having at least 80% amino acid sequence identity to SEQ ID Nos. 1-36 or 74-91 or an antibody or antibody fragment thereof specific for one of SEQ ID Nos. 1-36 or 74-91. In embodiments, the mammal is a primate. In embodiments, the primate is a human. In embodiments, the RNA that triggers RNAi comprises small interfering RNAs (siRNA). In embodiments, the siRNA comprises microRNA (miRNA) or a binding site for miRNA. In embodiments, the miRNA binds to the S′UTR of RNA encoding one of SEQ ID Nos. 1-36 or 74-91. In embodiments, the RNAi binds to the 3′UTR of RNA encoding a polypeptide having at least 80% amino acid sequence identity to SEQ ID Nos. 1-36 or 74-91. In embodiments, the composition is locally administered, e.g., to the lungs. In embodiments, the composition is systemically administered or intranasally administered. The composition can comprise liposomes or nanoparticles comprising the siRNA. The antibody fragment can comprise Fab′, F(ab′)2, scFv or a single domain, e.g., of a heavy chain or light chain.

Described herein are methods to detect influenza virus in a sample, comprising: detecting in a biological sample the presence or amount of a polypeptide having at least 80% amino acid sequence identity to one of SEQ ID Nos. 1-36 or 74-91 or a portion thereof with the activity of SEQ ID Nos. 1-36 or 74-91.

### BRIEF DESCRIPTION OF FIGURES

FIG. 1 illustrates host factors that influence influenza virus replication.

FIG. 2 illustrates loss of function using CRISPRi and gain of function using CRISPRa.

FIG. 3 illustrates constructs in the NS gene segment for CRISPRa.

FIG. 4 illustrates screening.

FIG. 5 illustrates sequentially passaging.

FIG. 6 illustrates enrichment of TRIPC viruses.

FIG. 7 illustrates changes in the population over sequential passages.

FIG. 8 shows identification of a host factor that promotes viral replication.

FIG. 9 compares knock out screens versus virus driven selections.

FIG. 10 illustrates CRISPRa and CRISPRi.

FIG. 11 illustrates use of CRISPRa and CRISPRi and sgRNAs.

FIG. 12 illustrates use of an example CRISPRa and sgRNA.

FIG. 13 illustrates virus driven selection of host modifiers.

FIGS. 14A-14H illustrate how transcriptional regulation by influenza-programmed Cas9

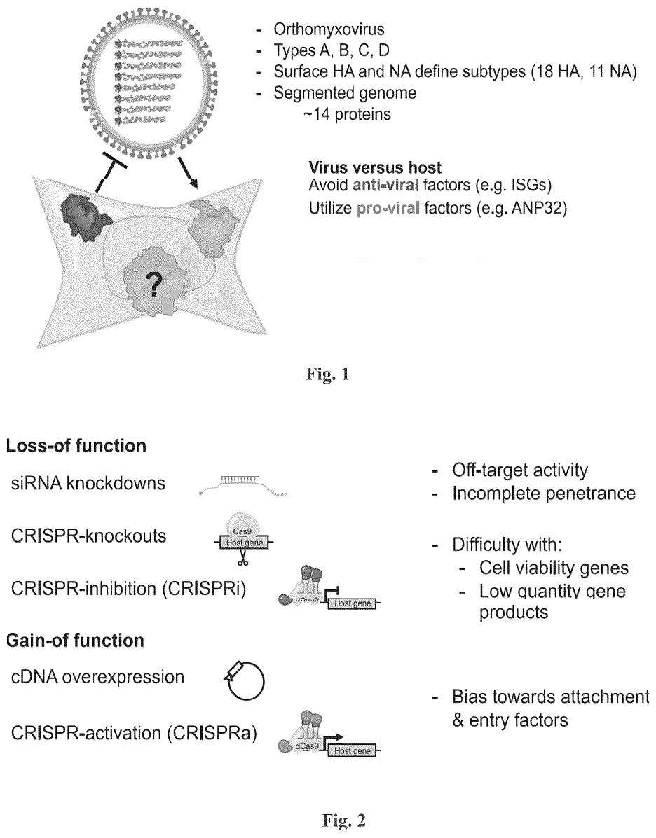

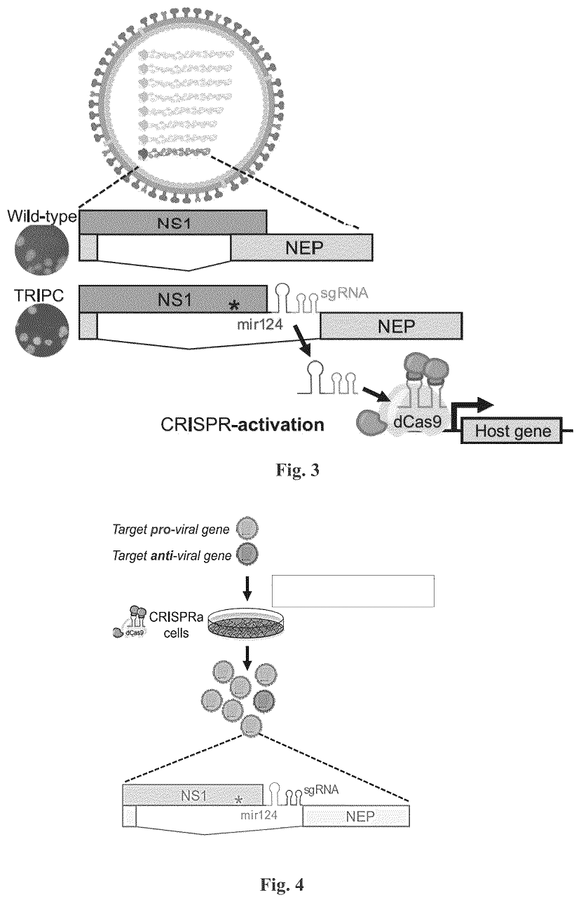

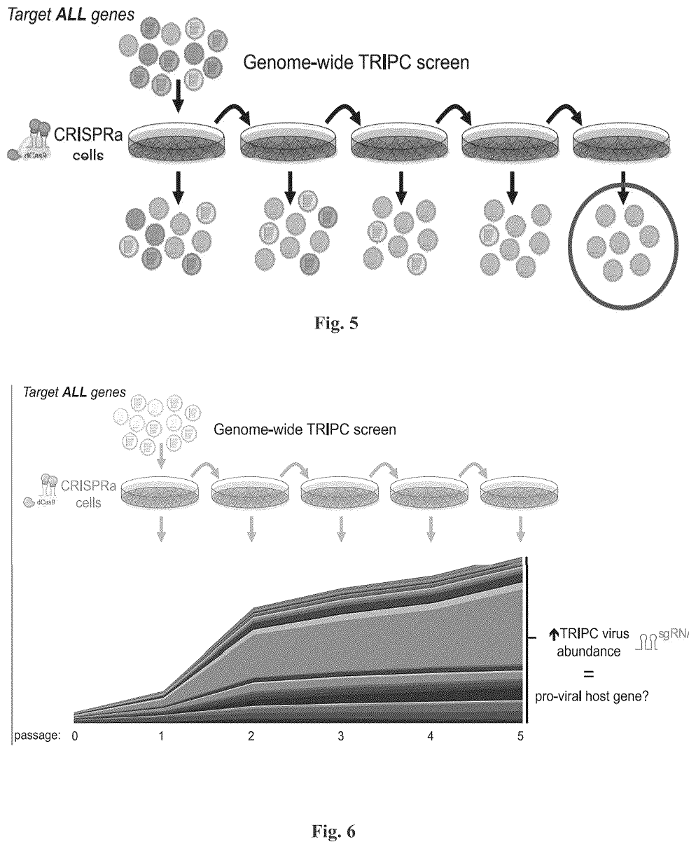

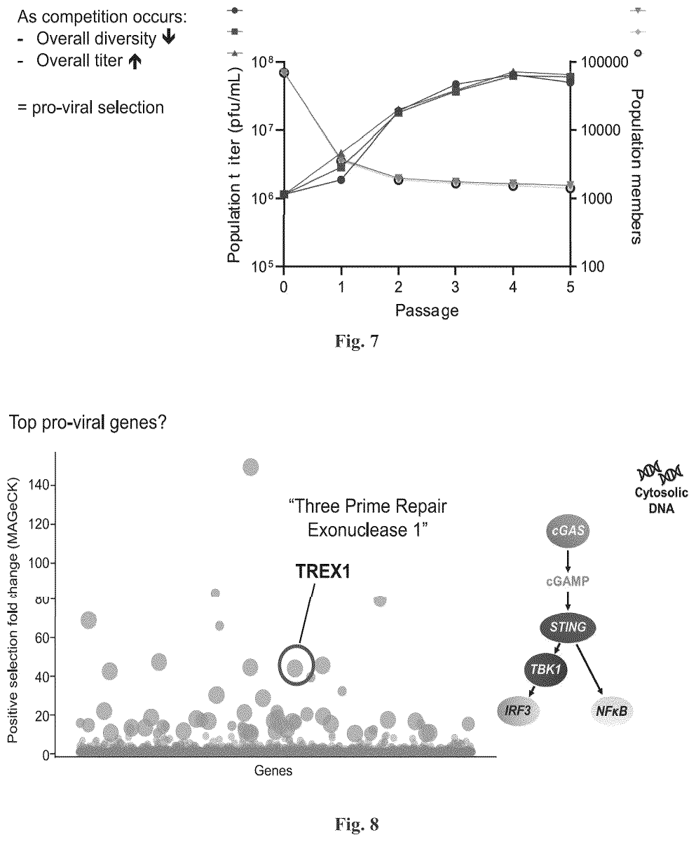

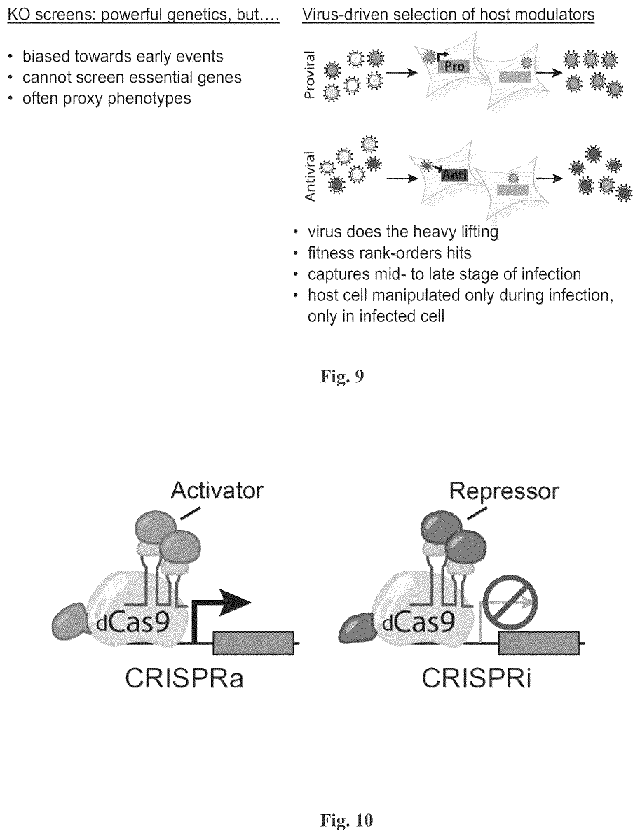

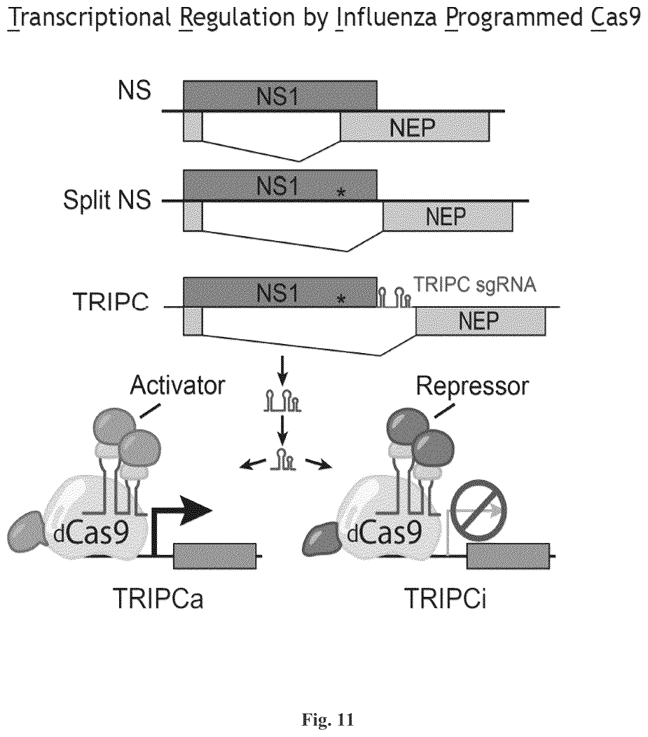

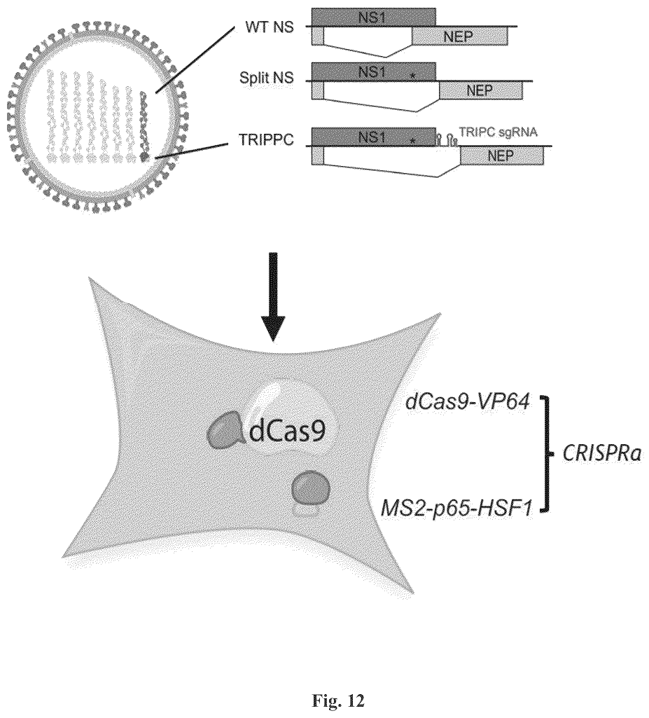

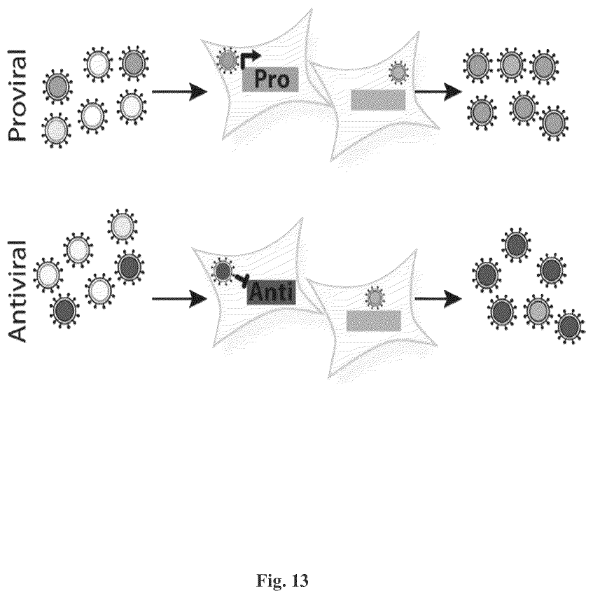

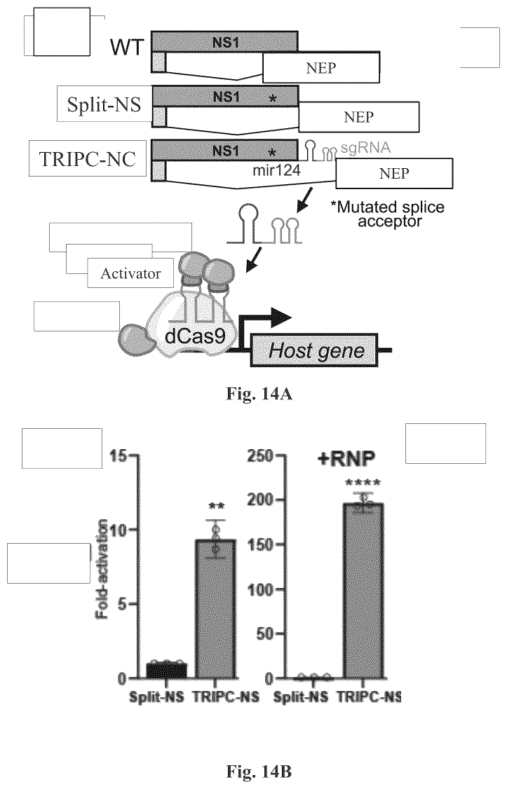

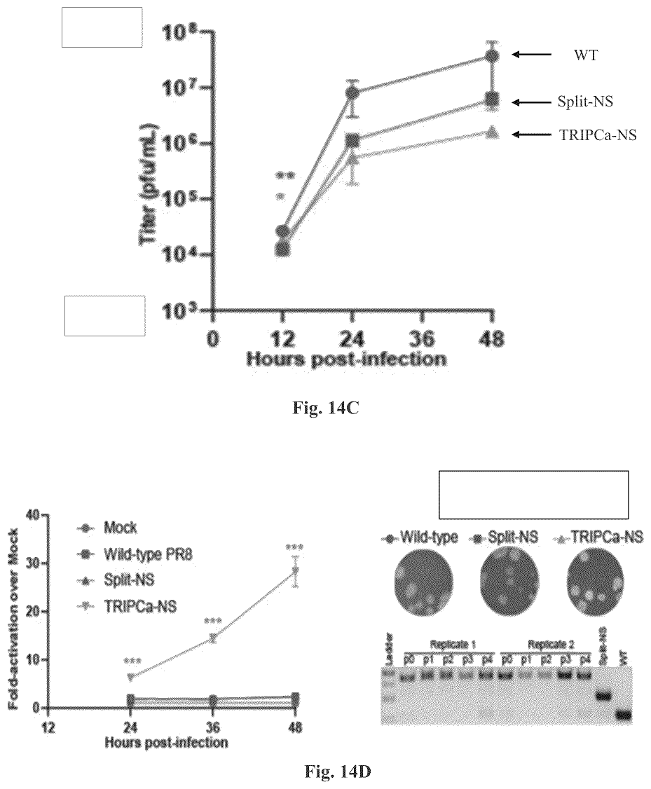

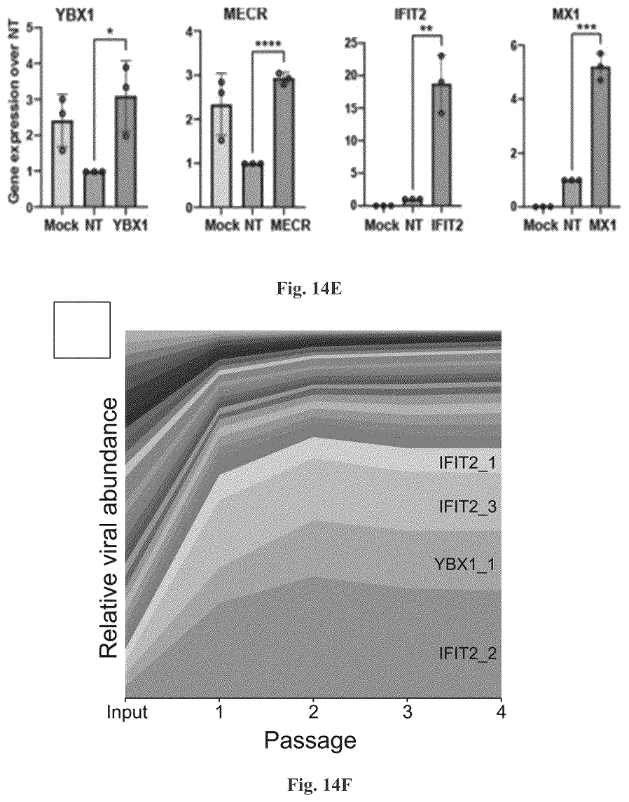

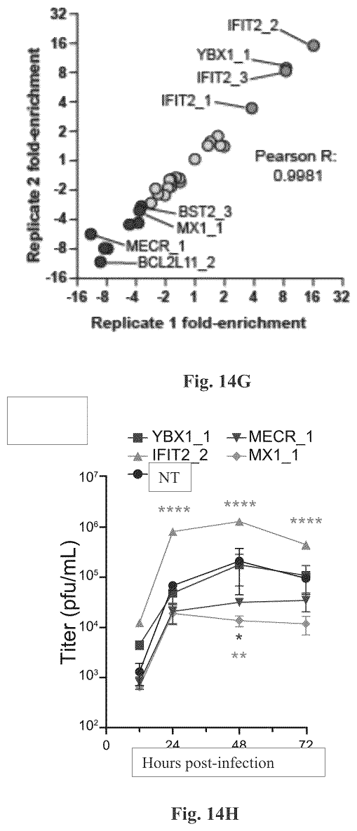

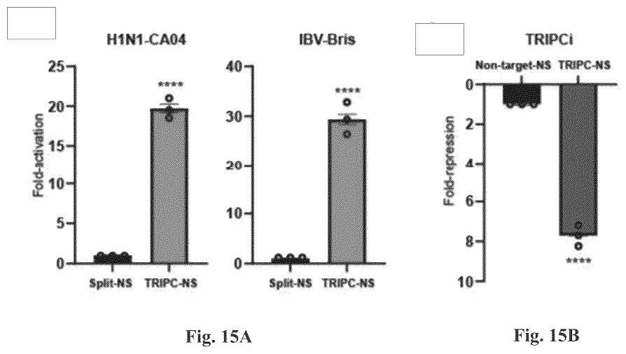

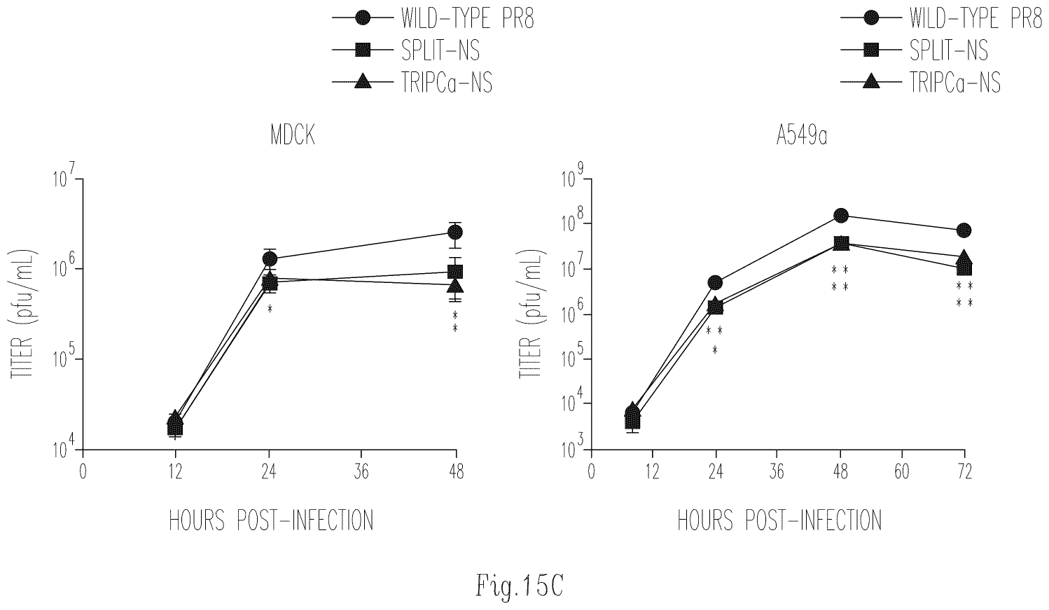

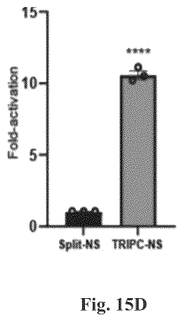

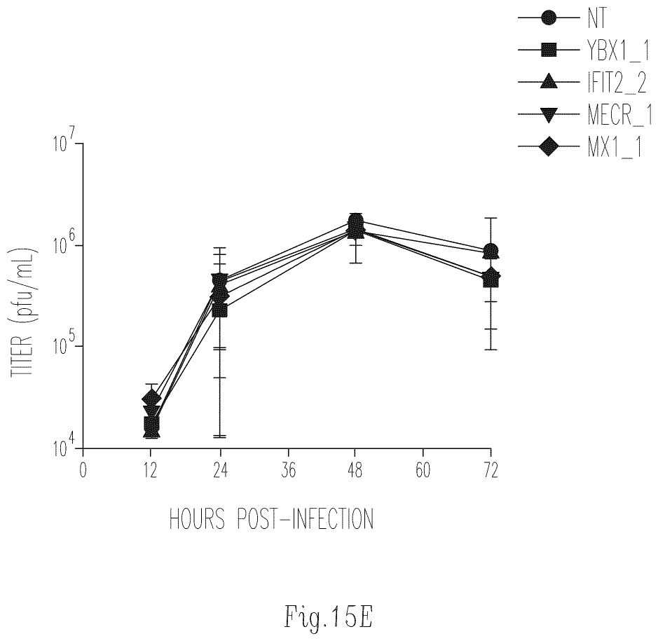

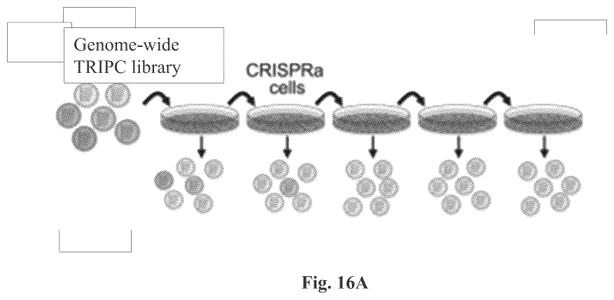

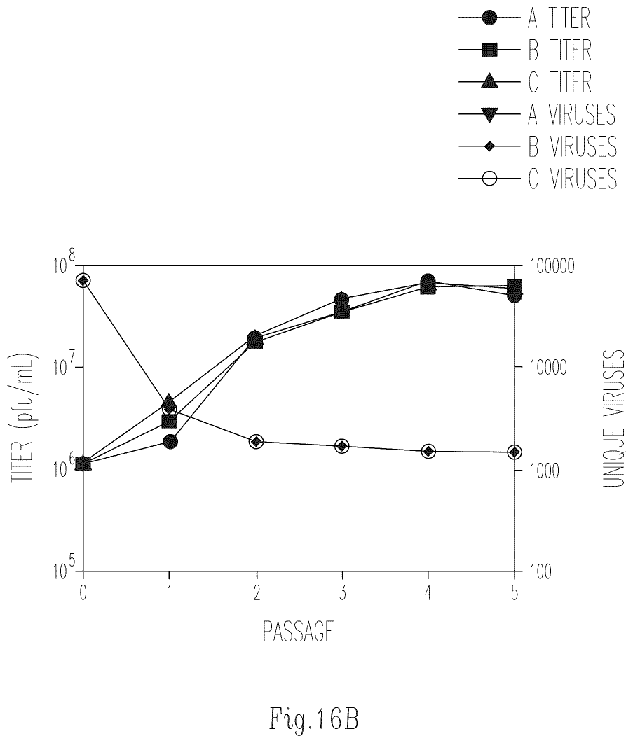

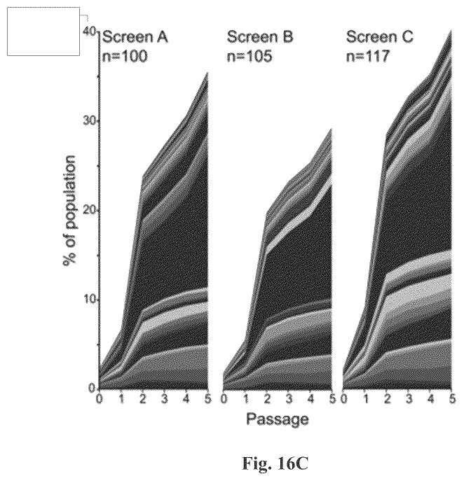

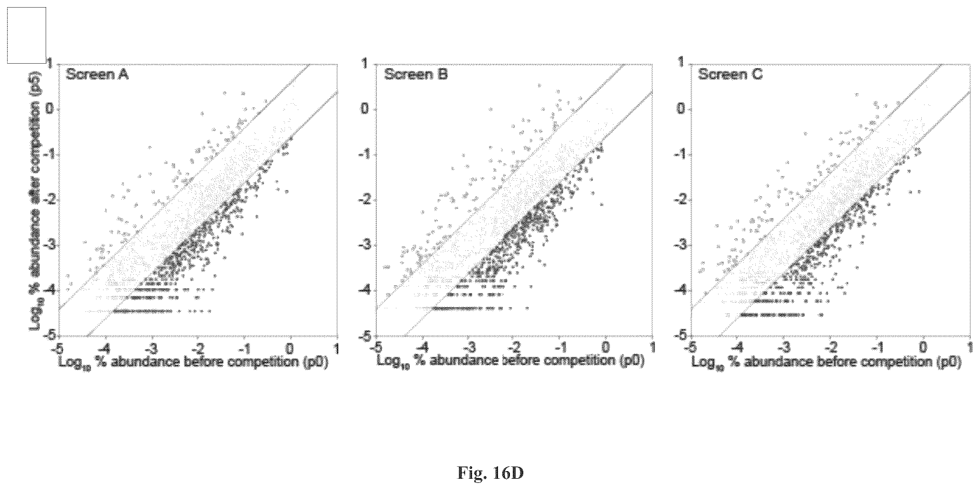

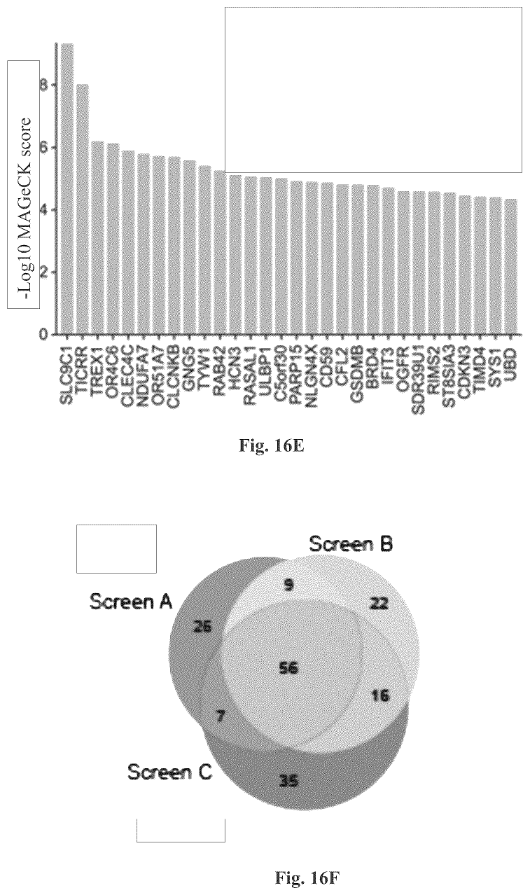

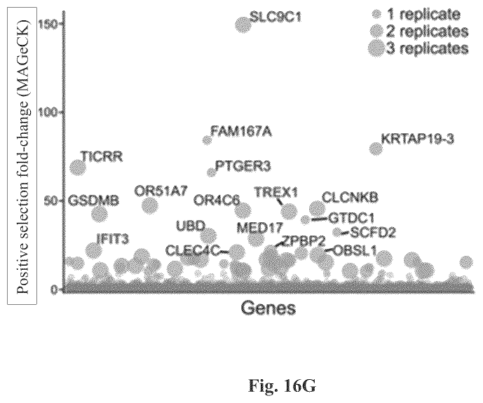

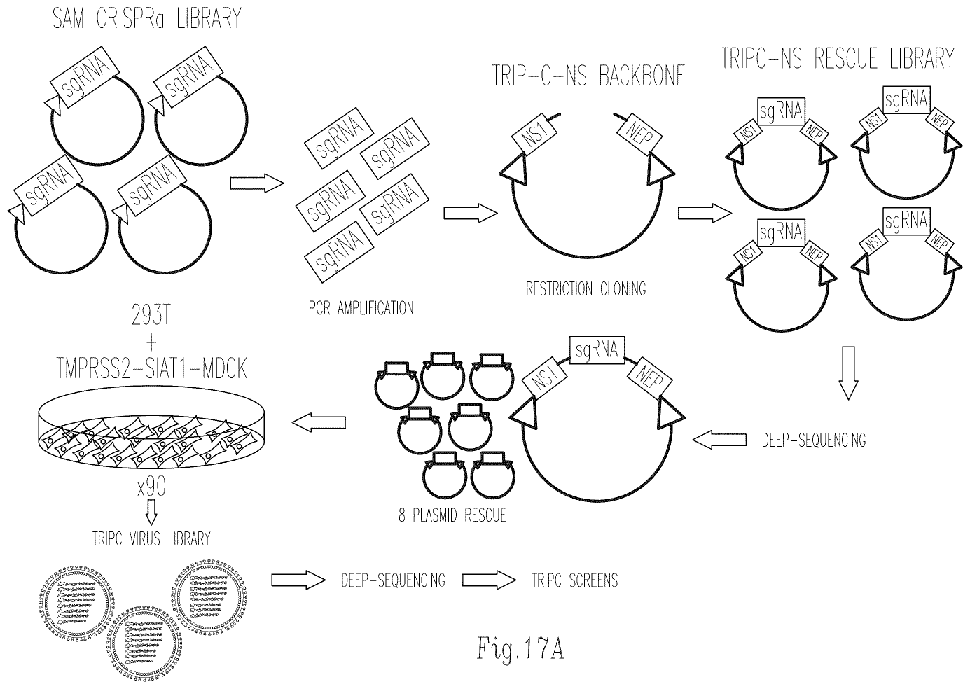

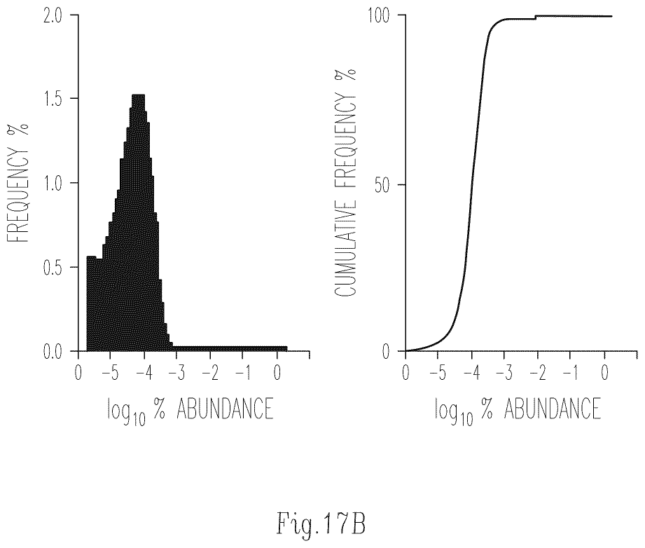

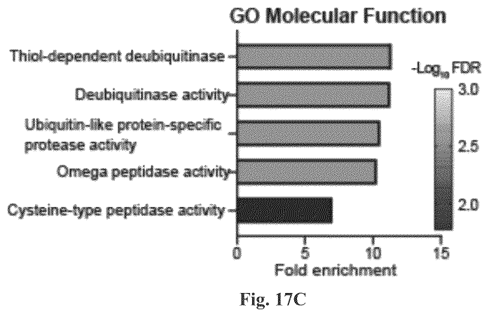

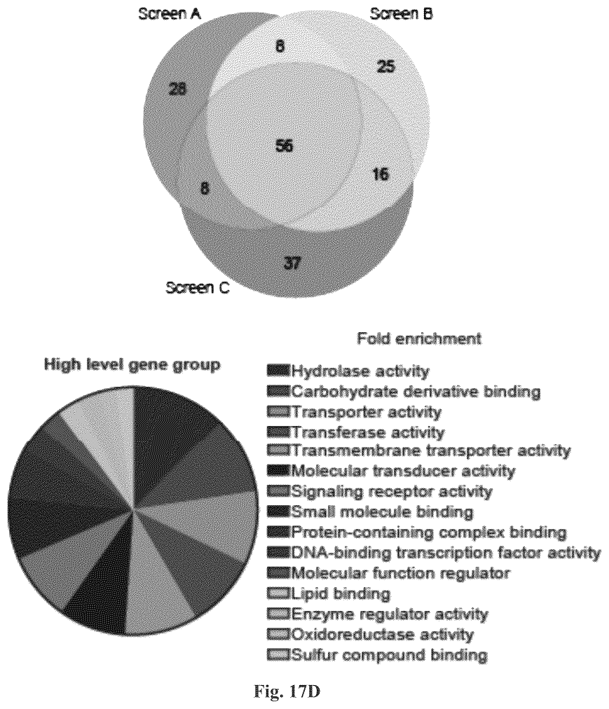

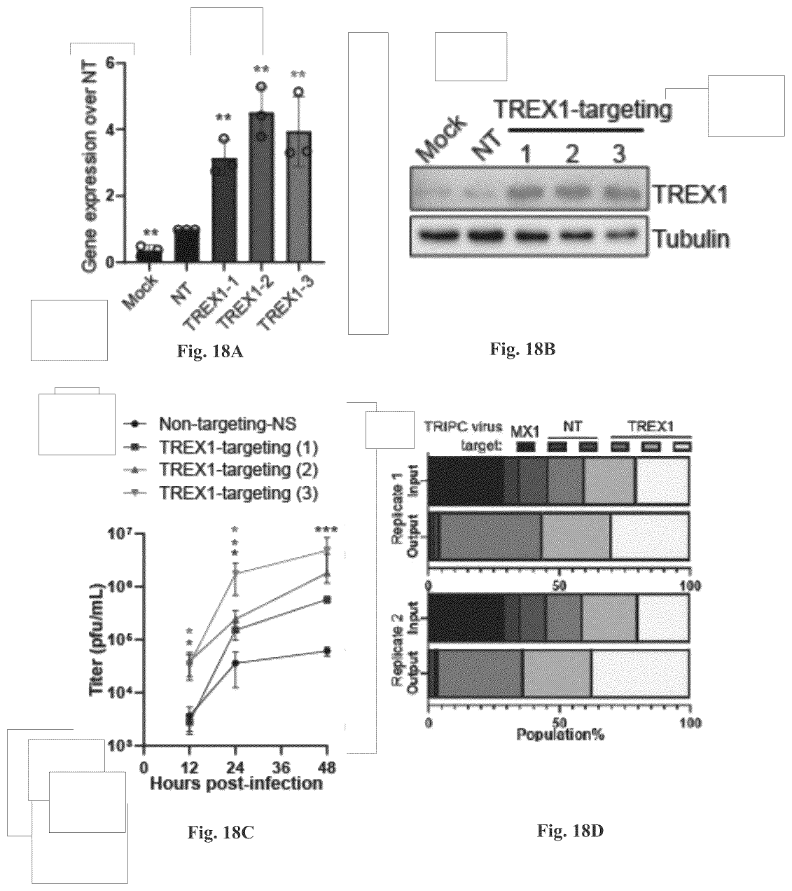

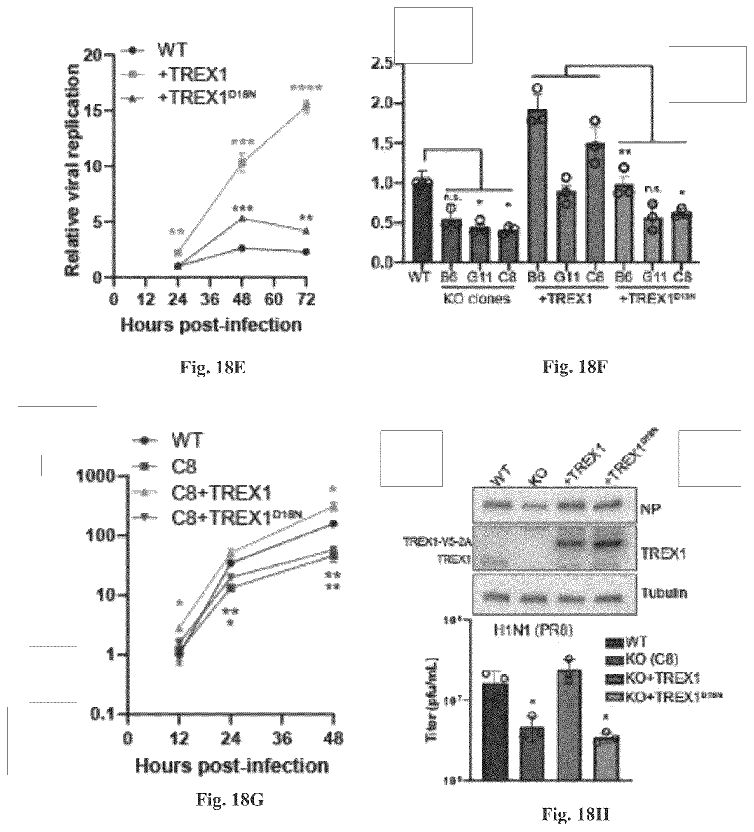

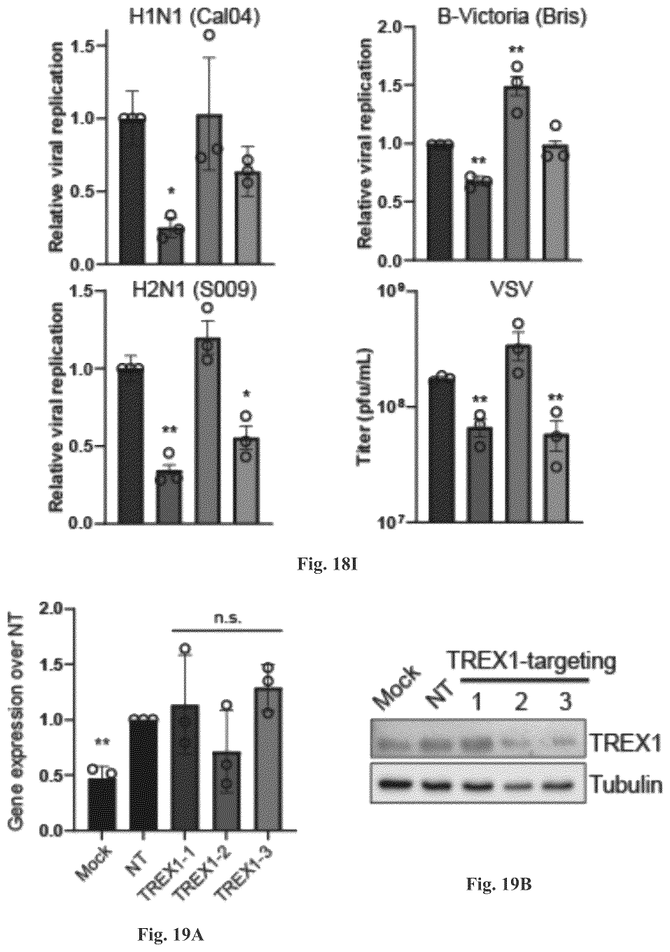

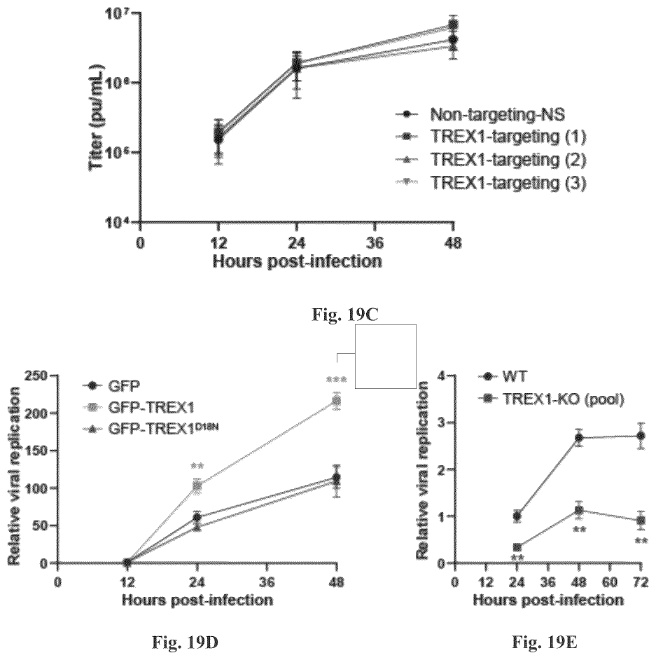

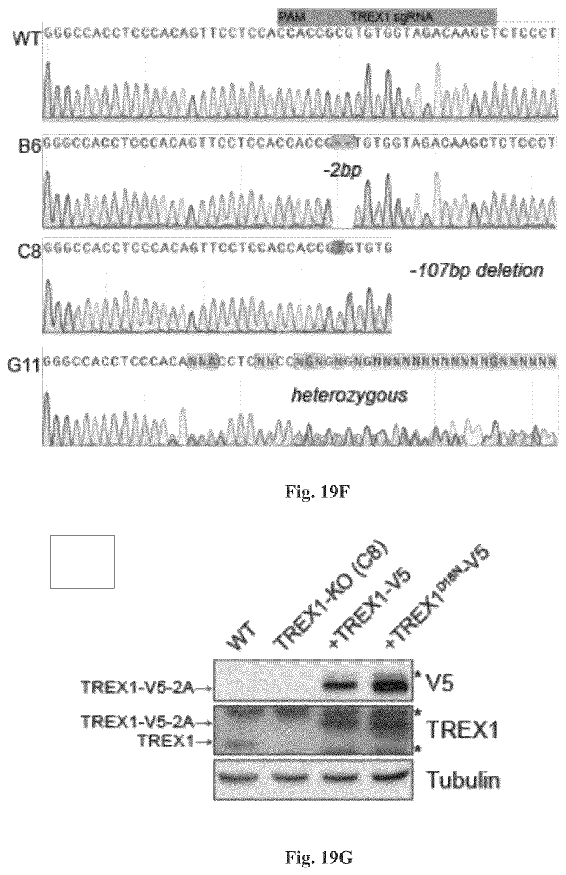

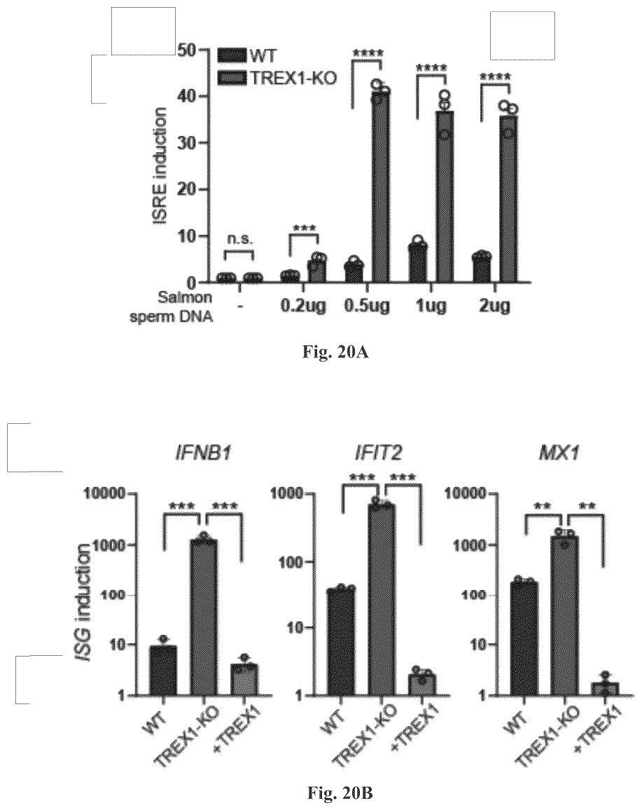

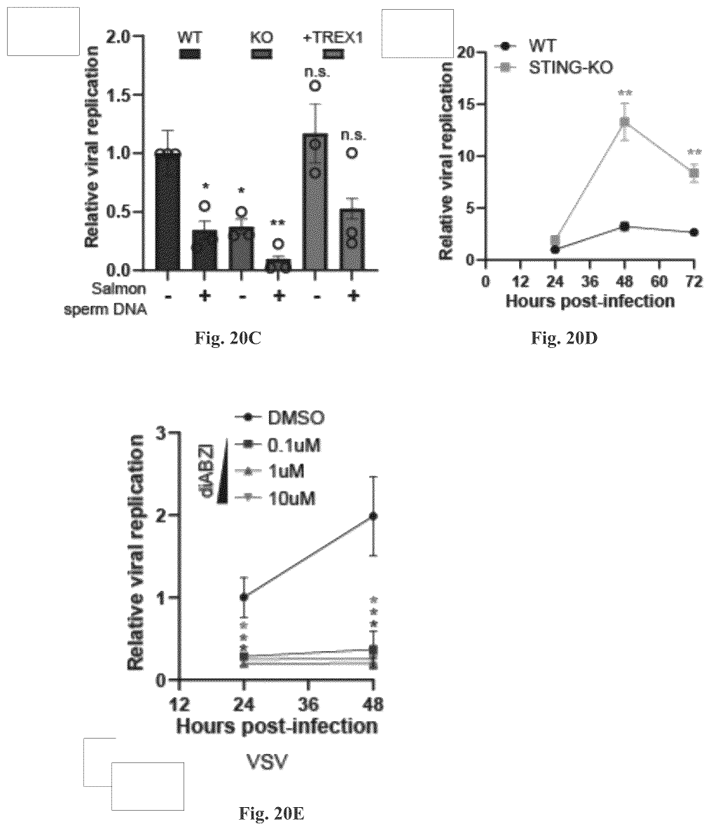

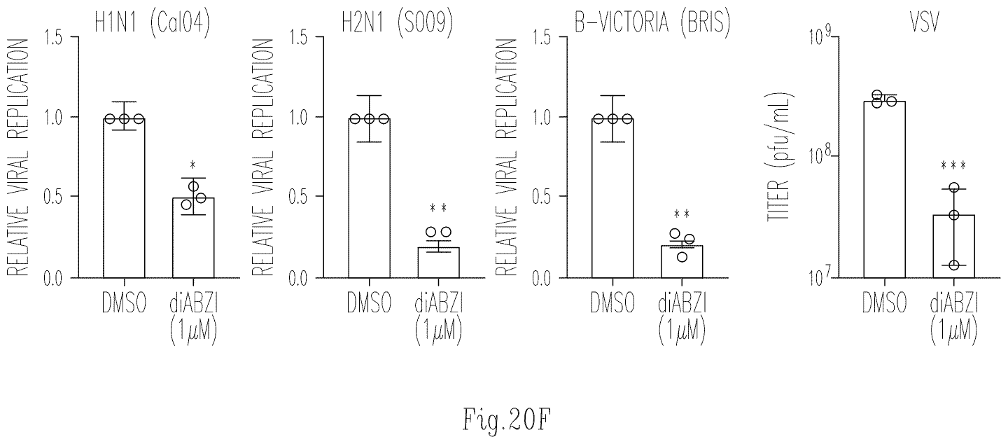

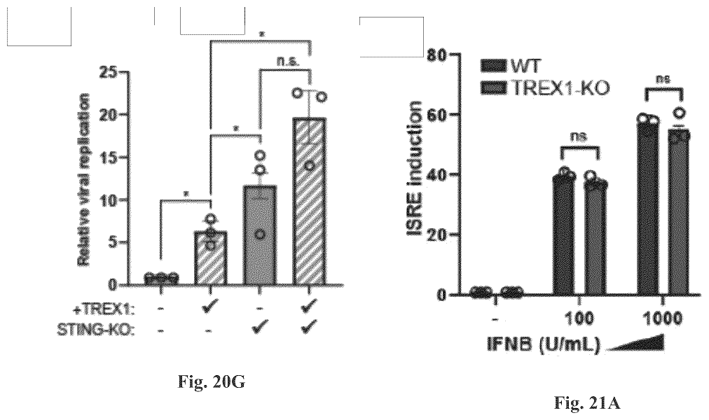

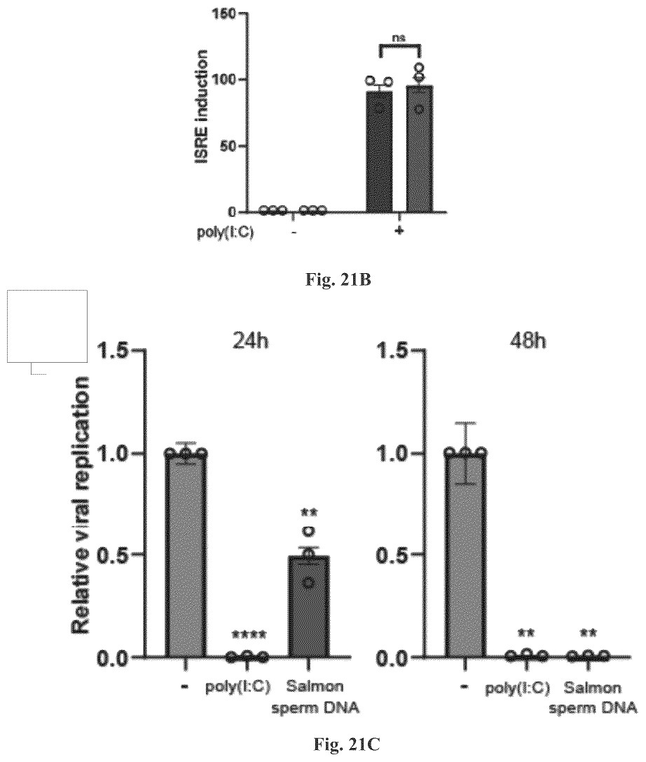

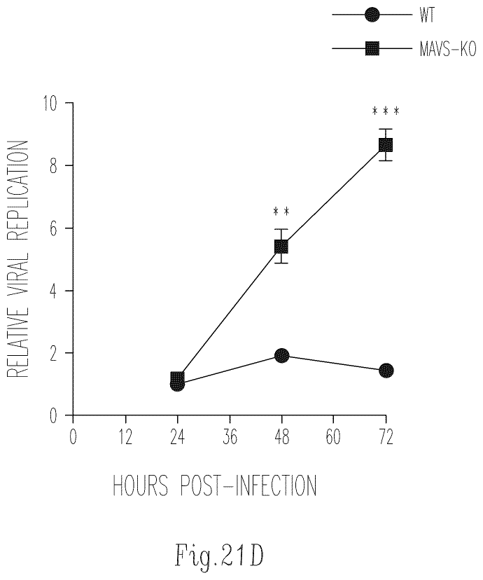

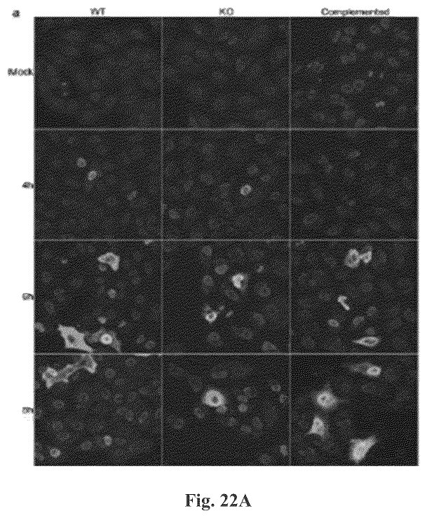

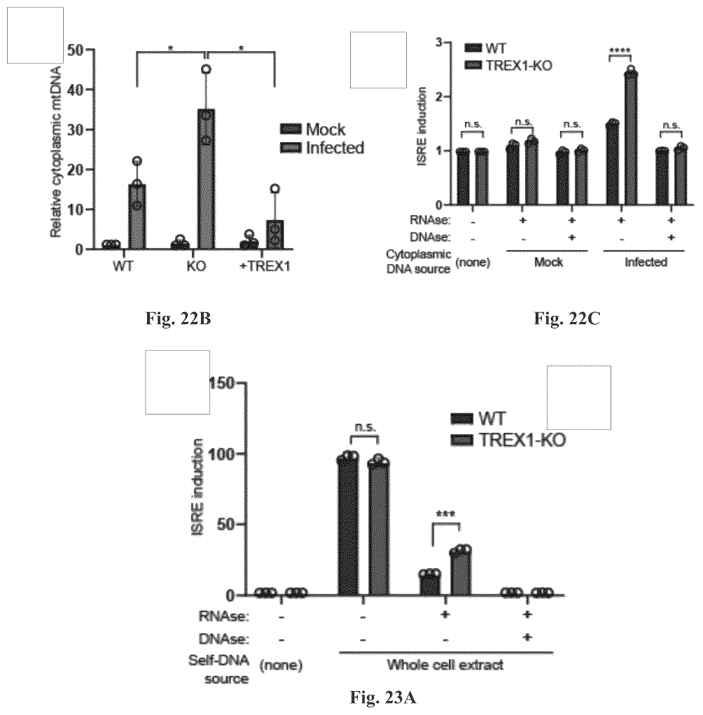

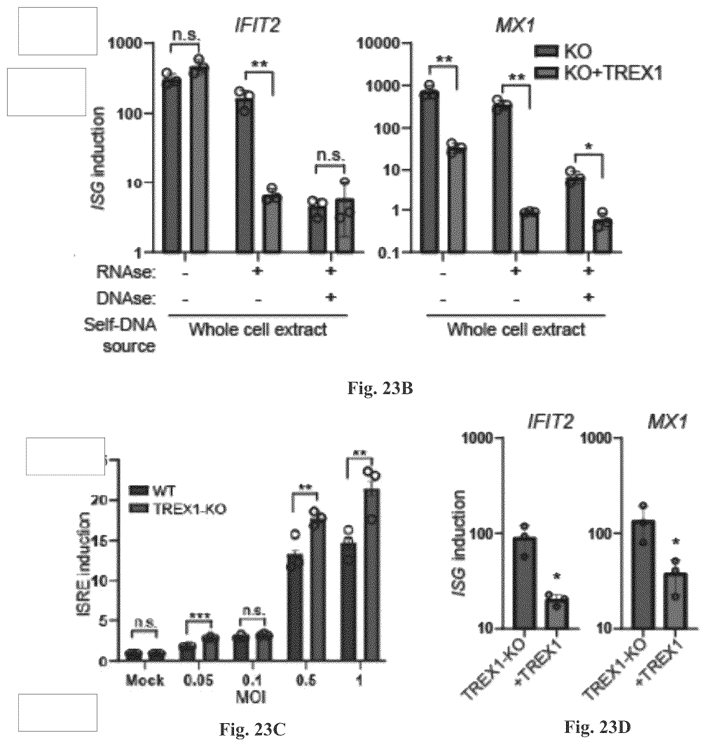

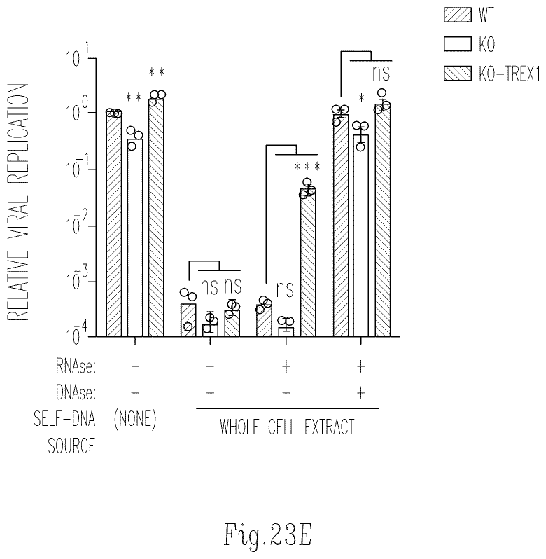

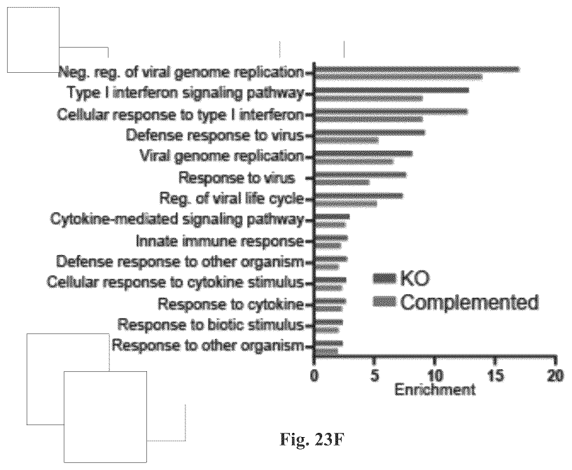

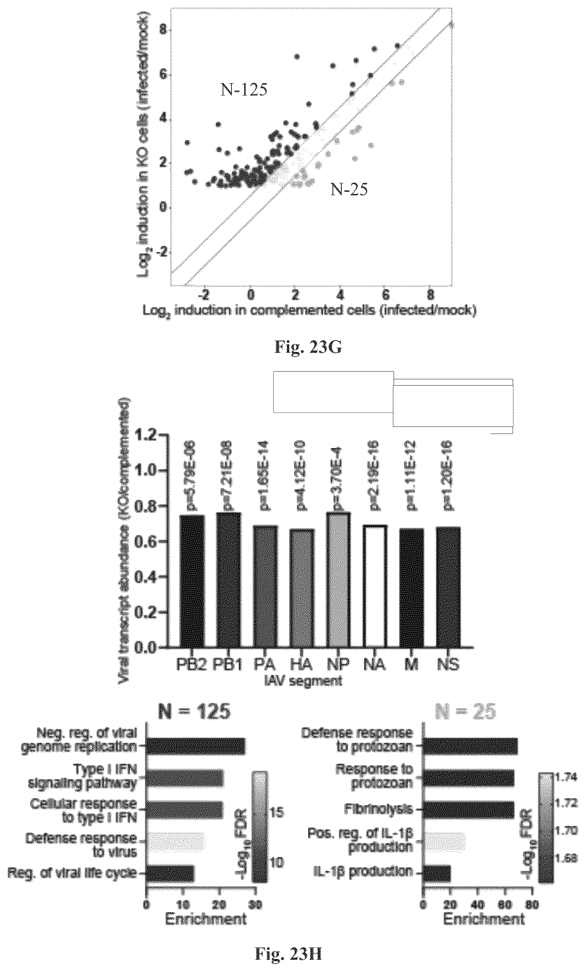

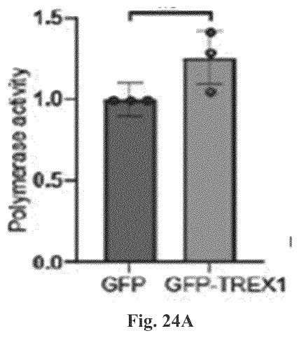

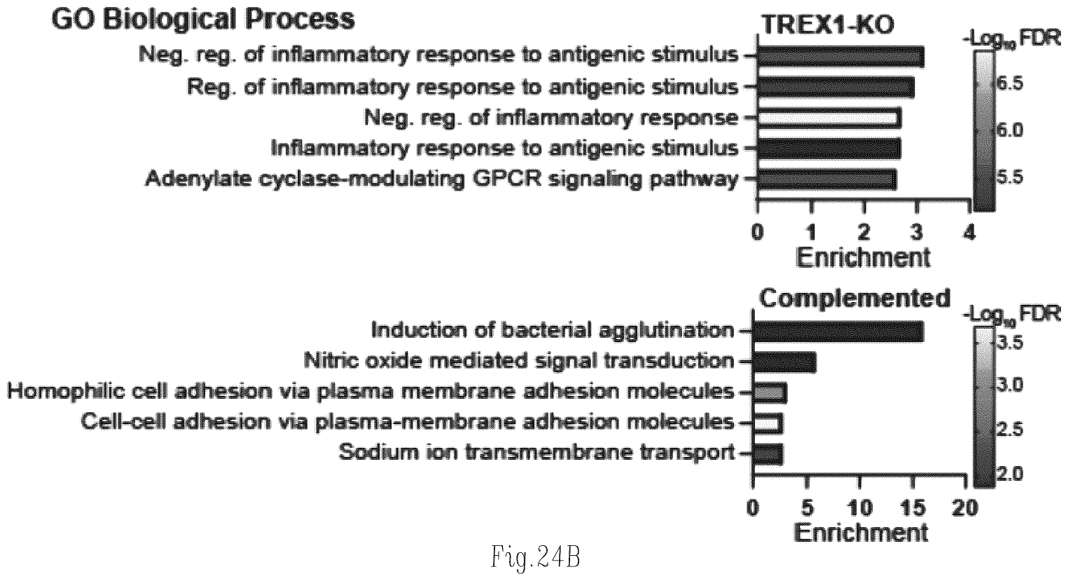

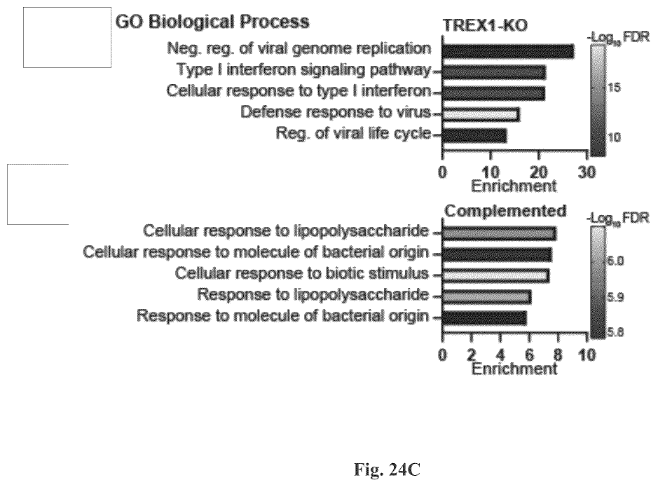

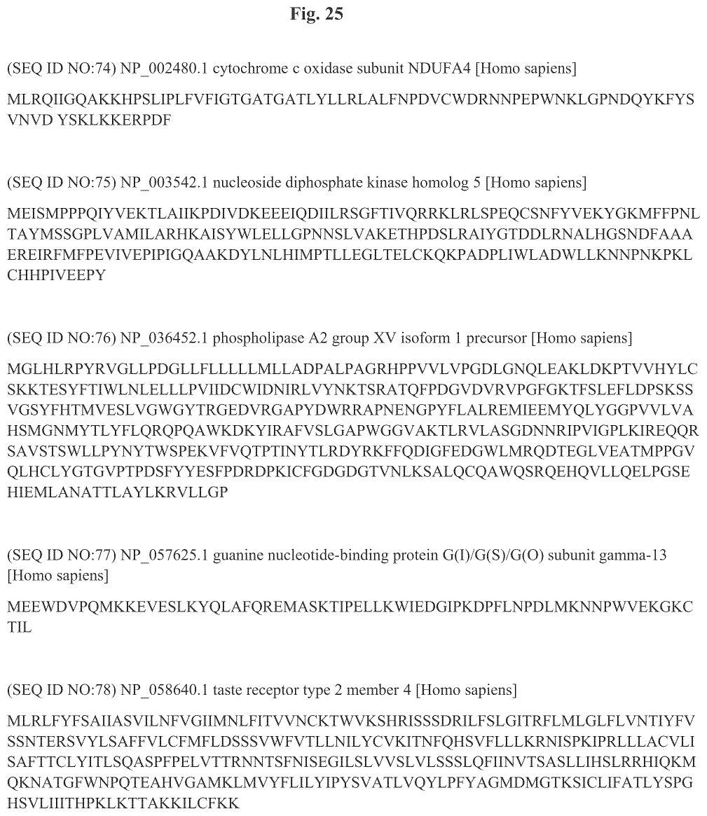

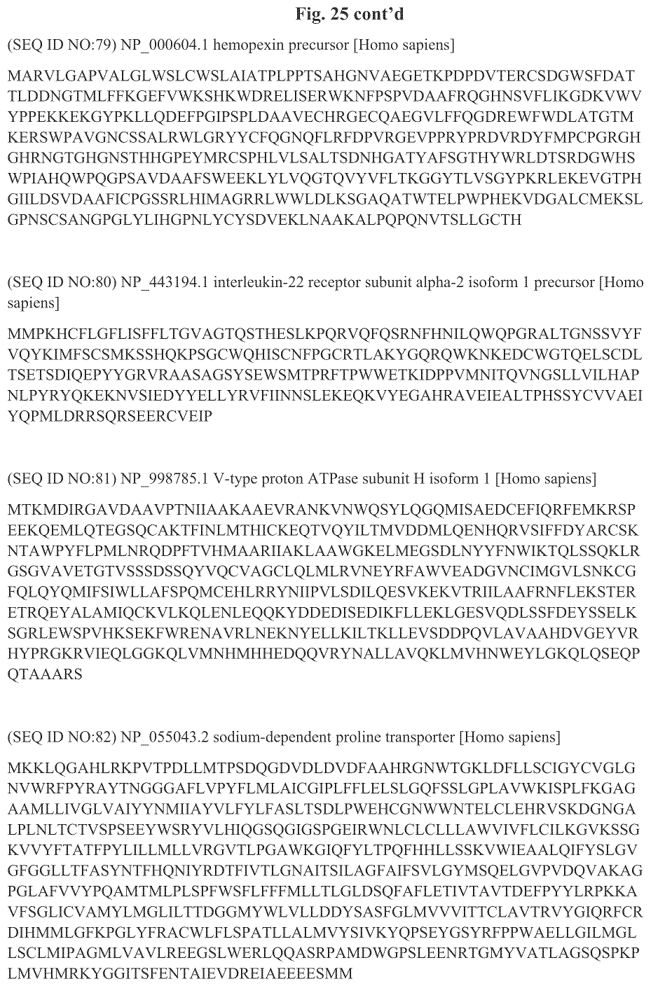

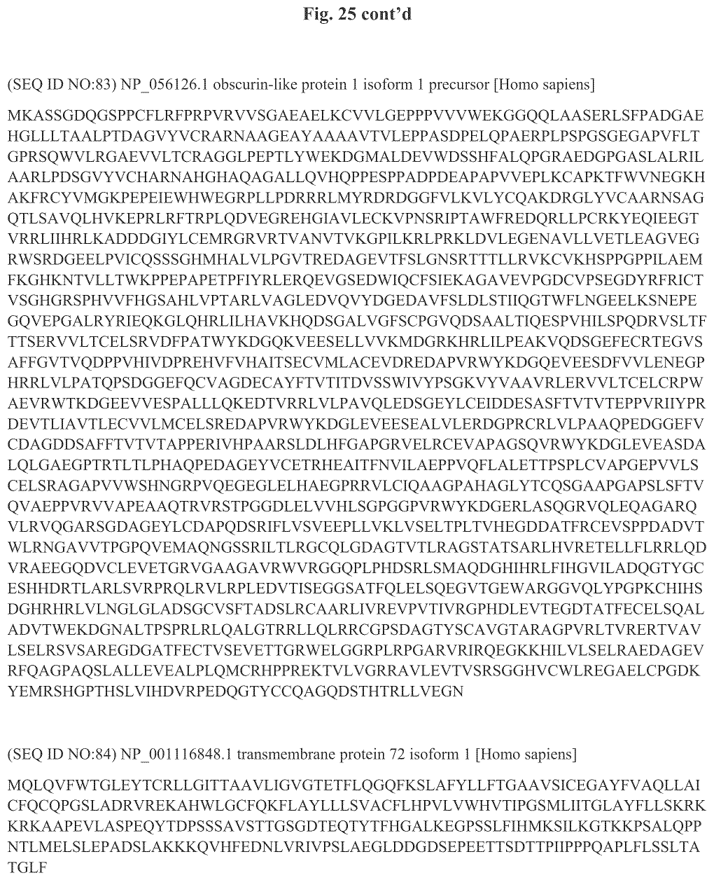

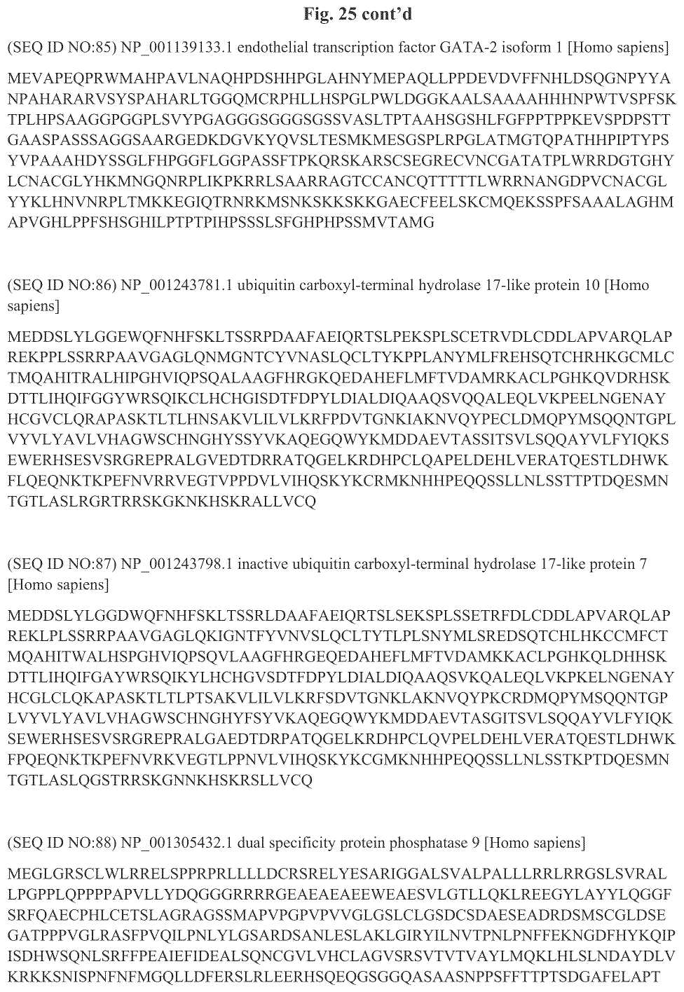

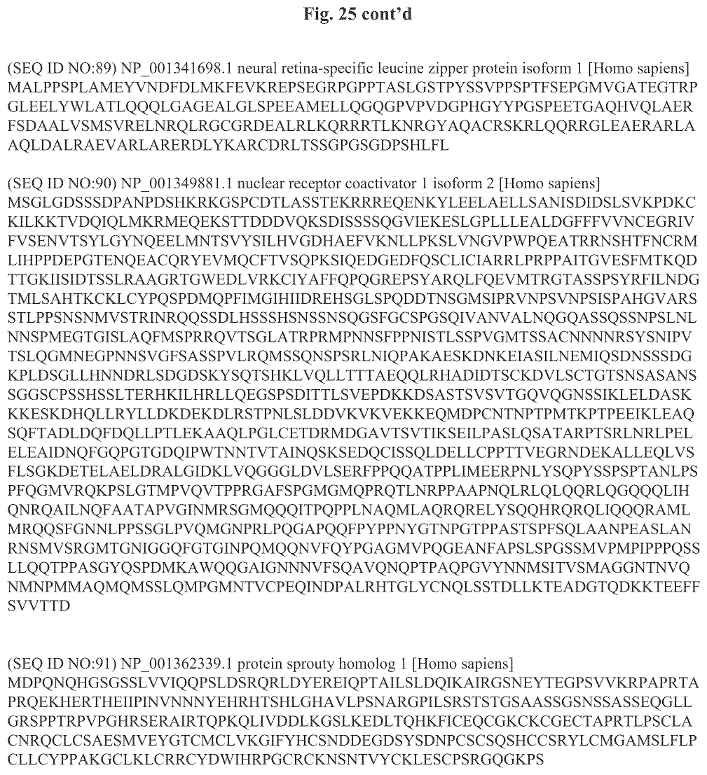

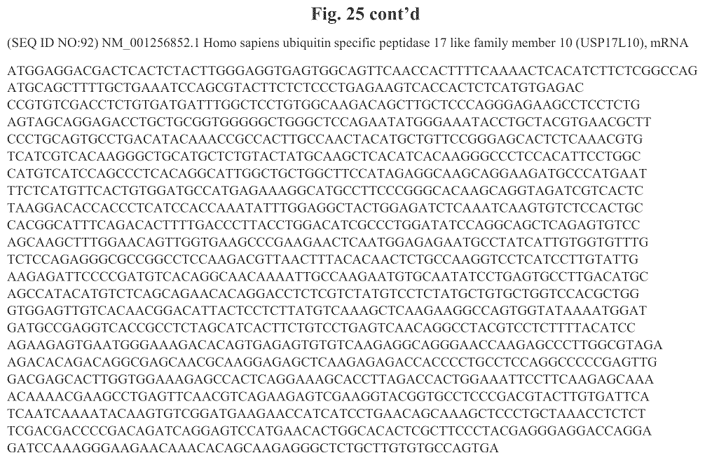

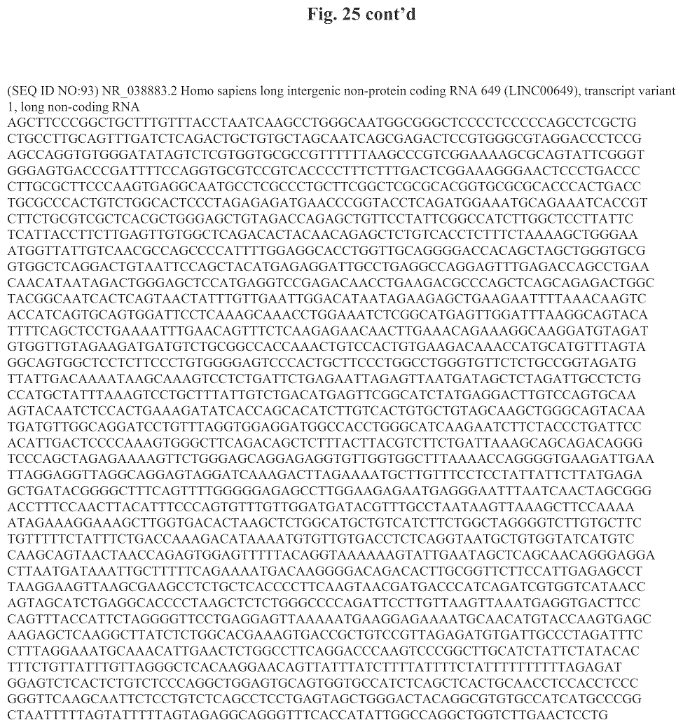

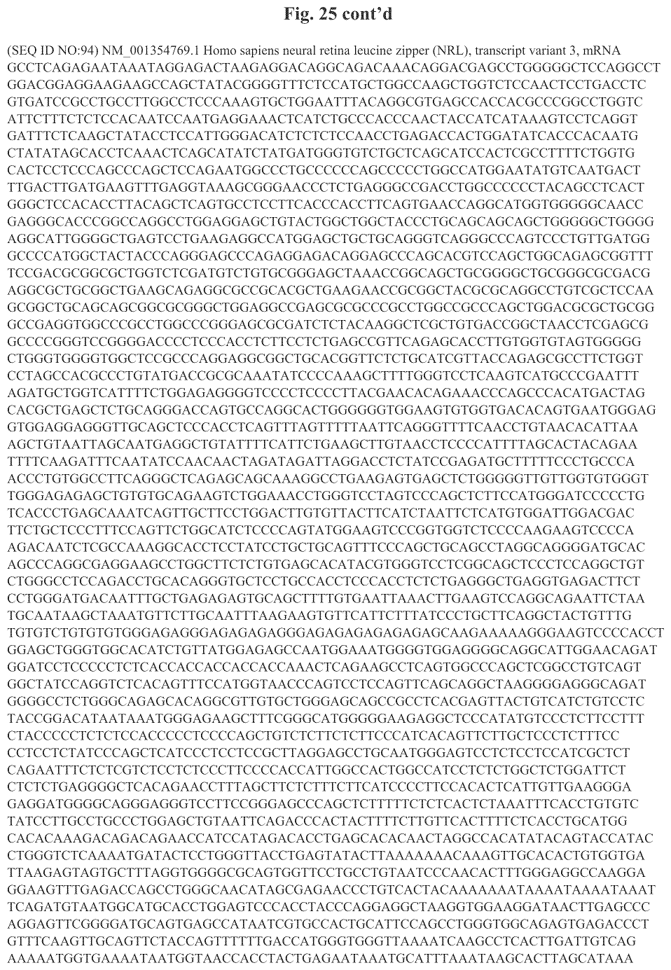

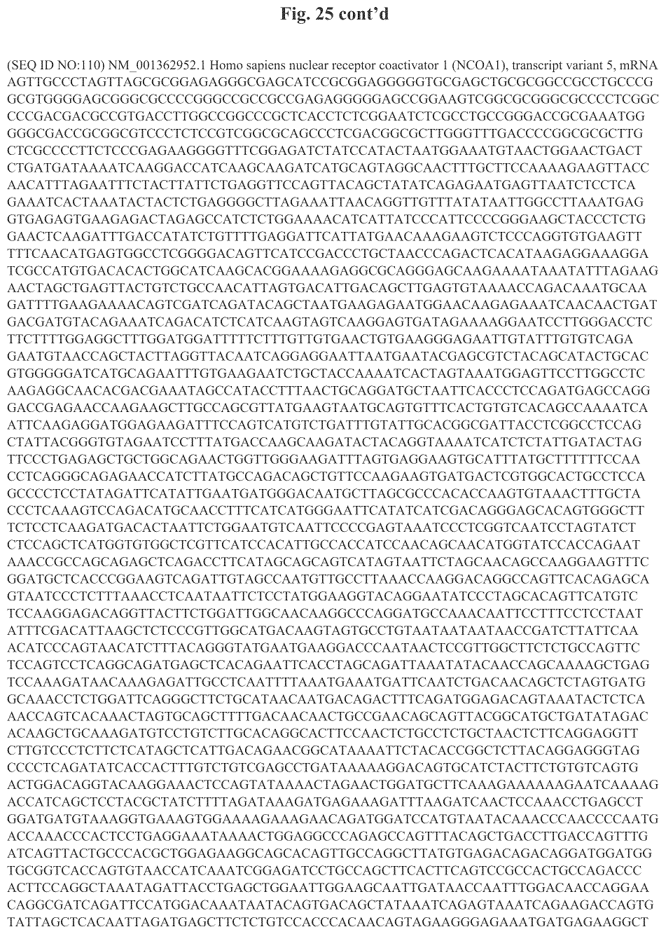

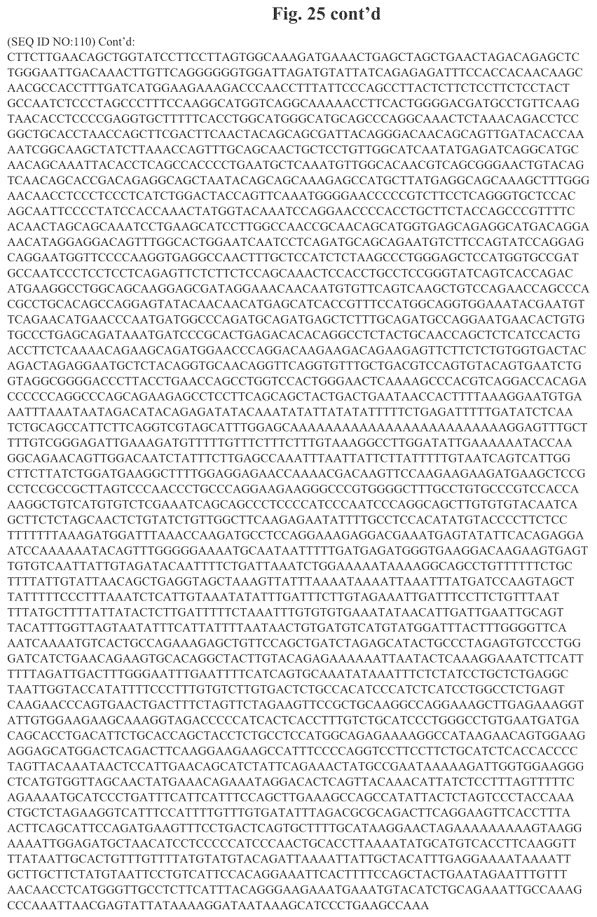

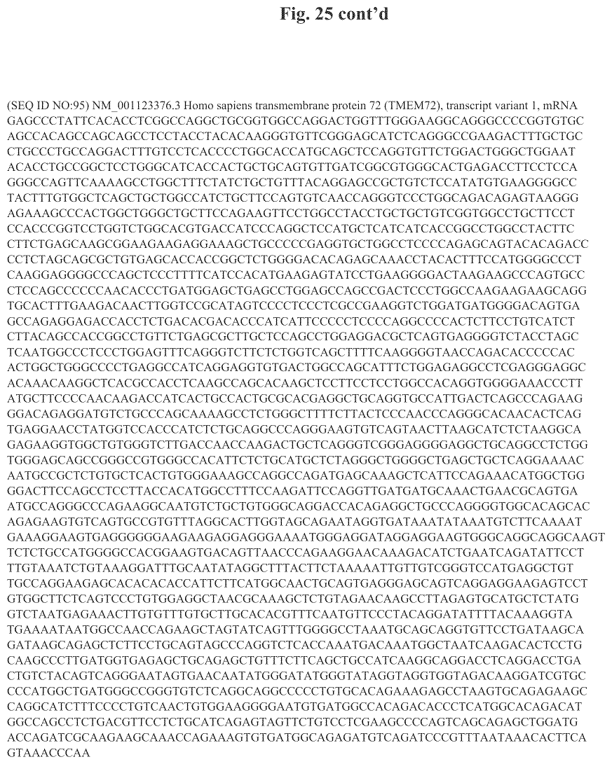

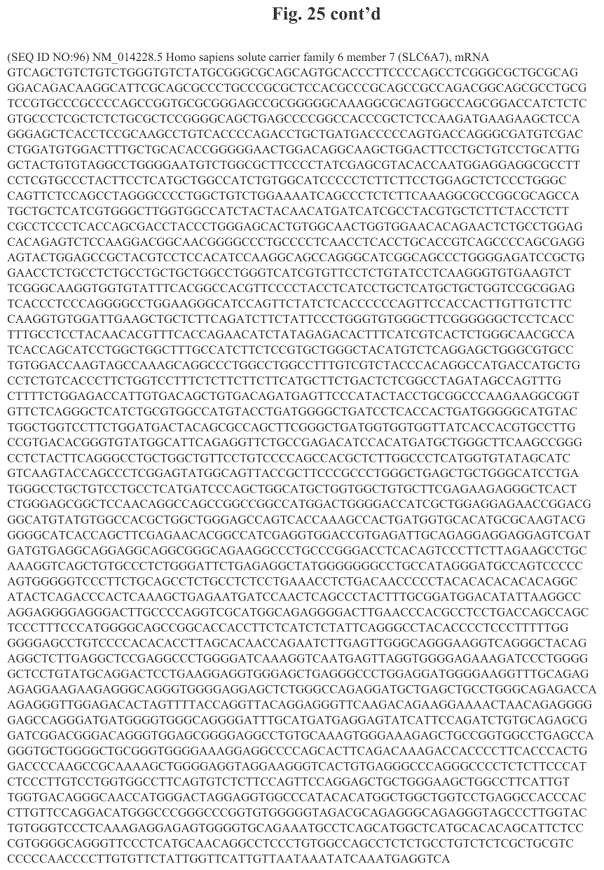

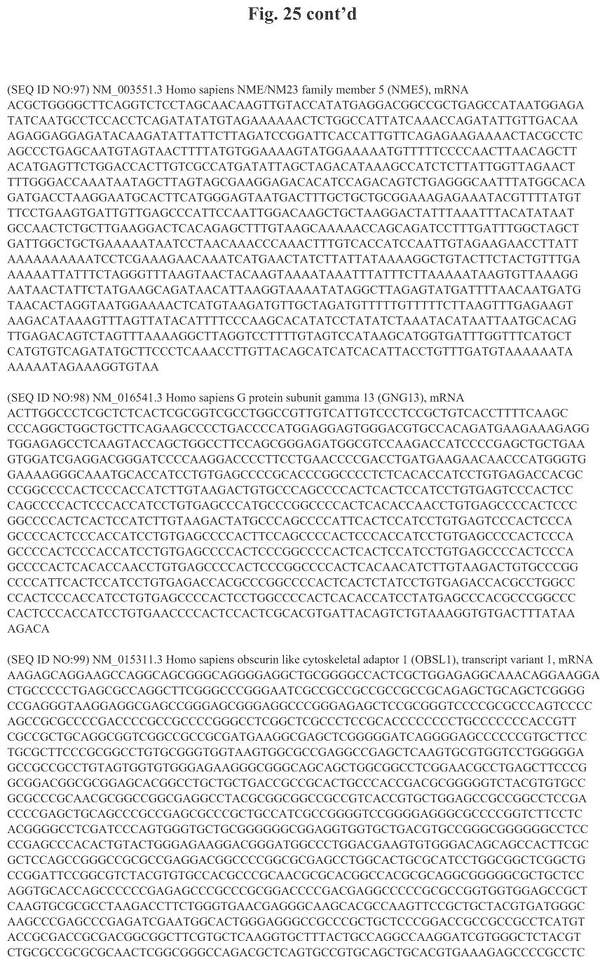

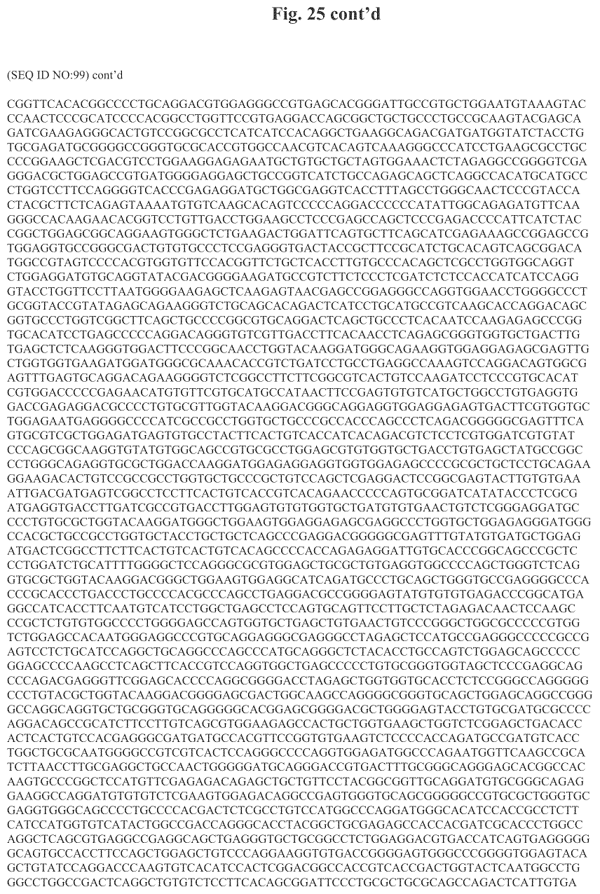

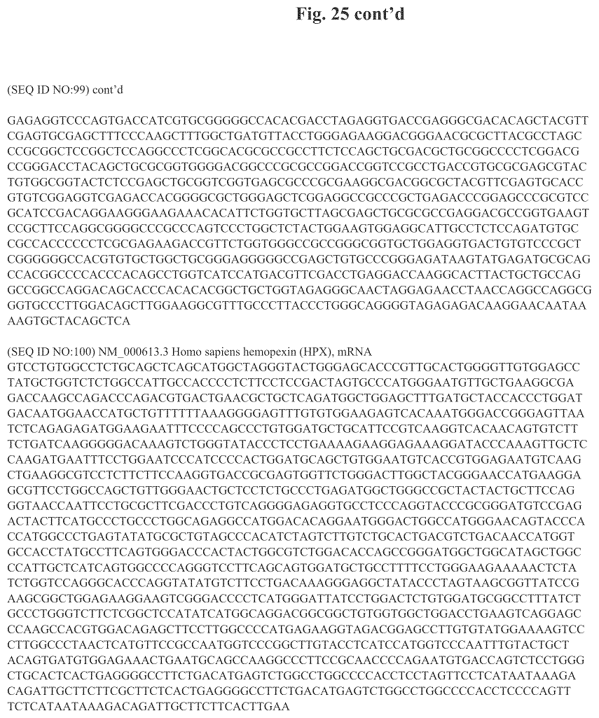

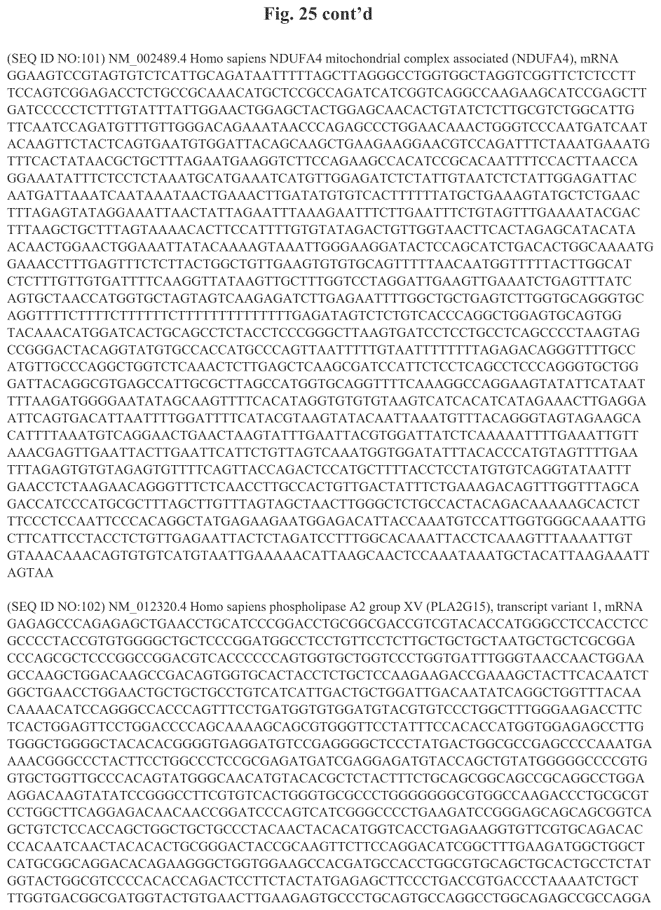

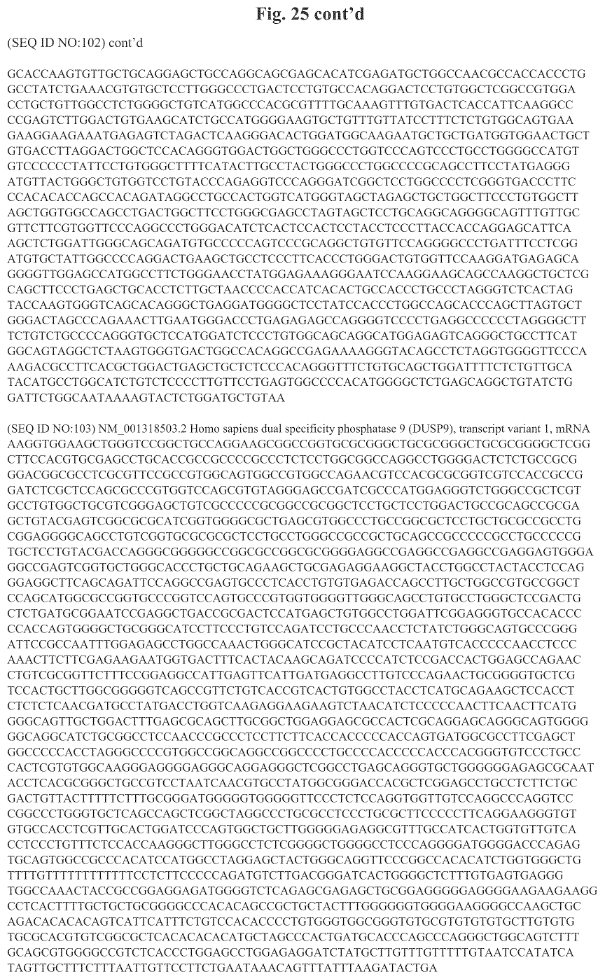

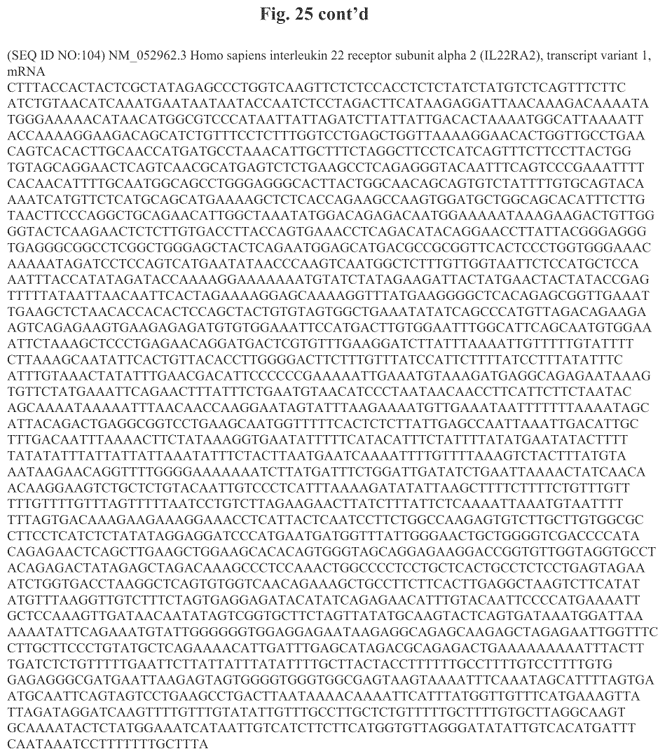

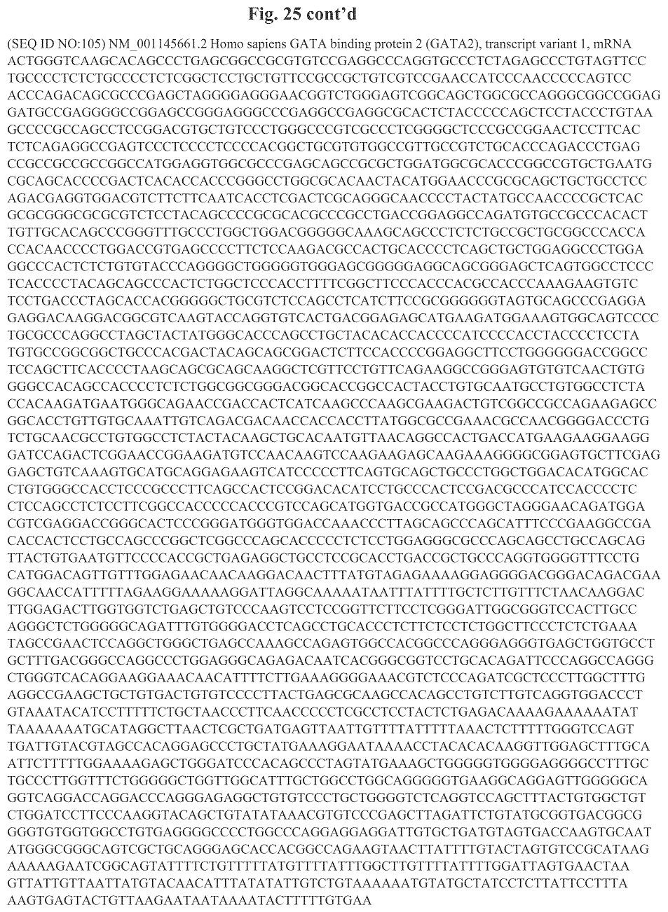

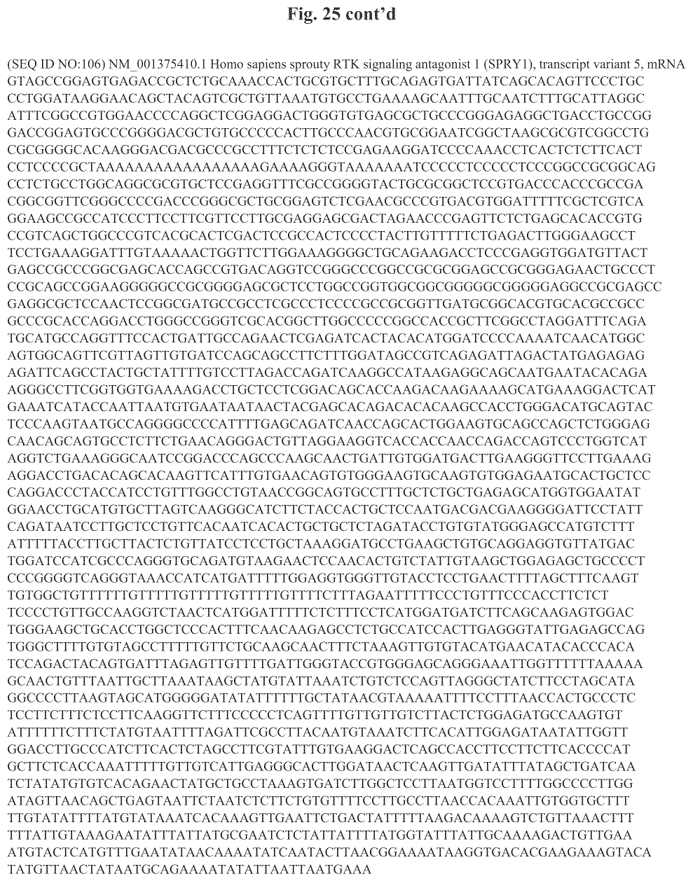

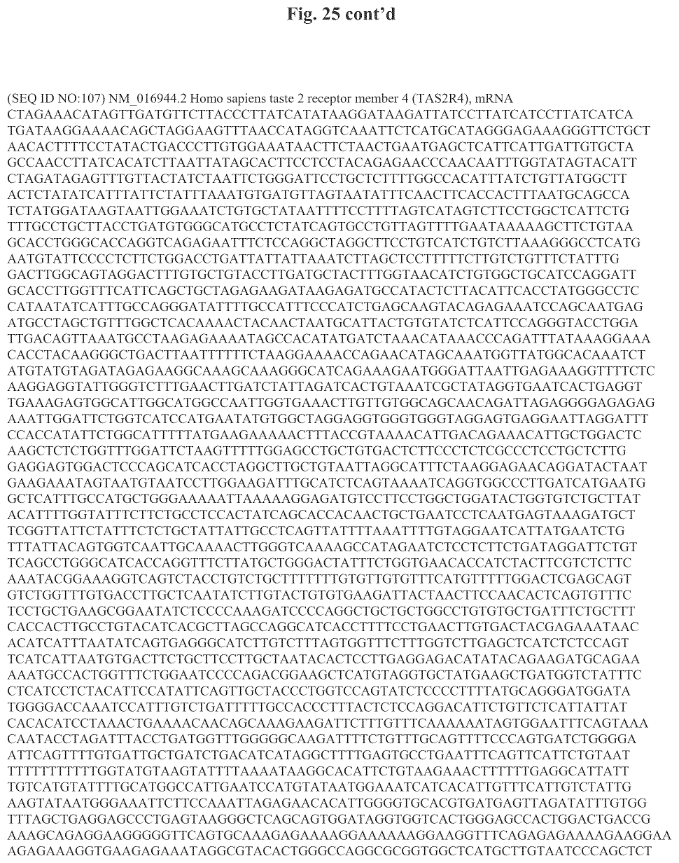

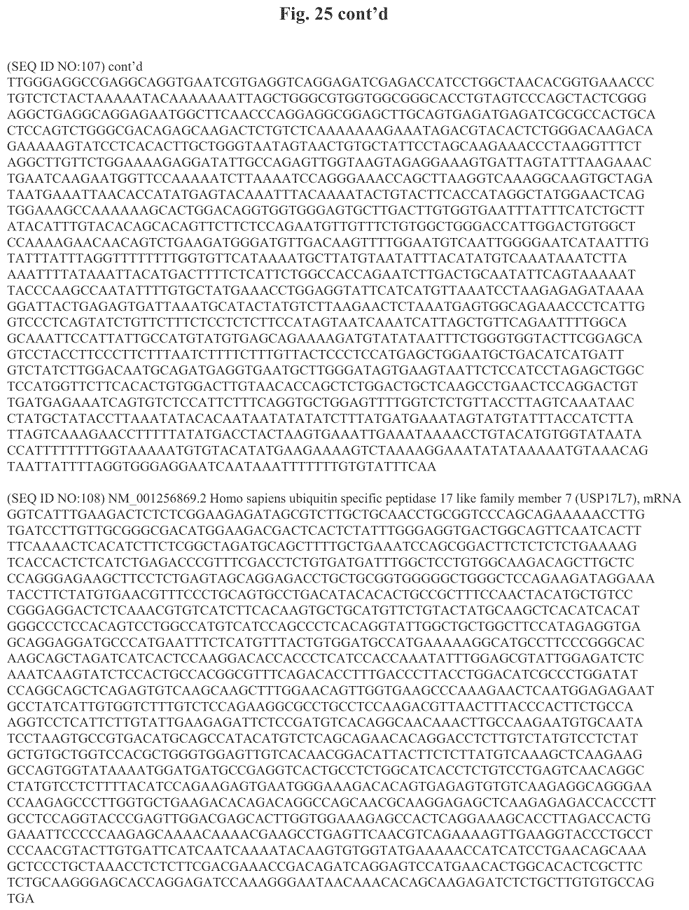

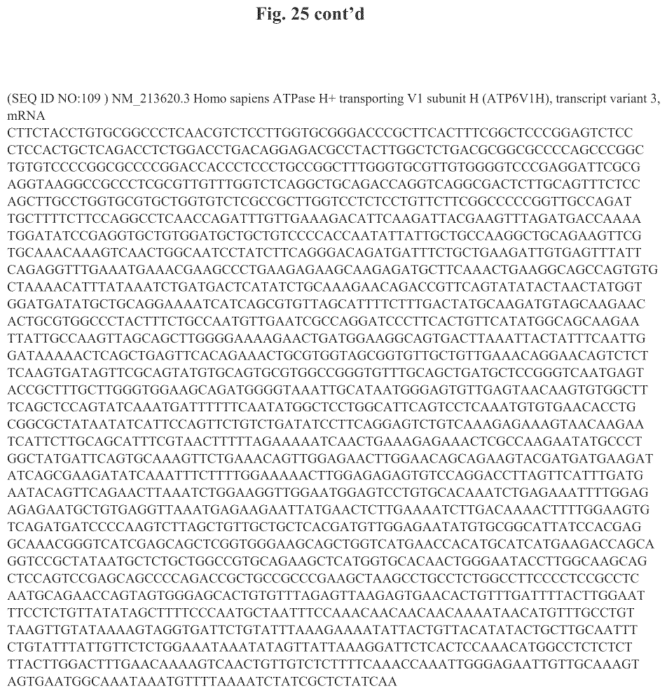

(TRIPC) manipulates host gene expression to enable fitness-based screening. FIG. 14A: Engineering influenza A virus (IAV) to express an sgRNA. Cartoon detailing engineering of the NS genome segment to encode and process the sgRNA needed to program Cas9 for CRISPR activation (CRISPRa)-mediated gene expression. FIG. 14B: Validation of TRIPC in transfected cells. TRIPC activation (TRIPCa) of a luciferase reporter targeted by sgRNA expressed from transfected NS (left). Inclusion of the viral polymerase and NP (+RNP), which amplify NS transcription and replication, boosts TRIPCa (right). FIG. 14C: TRIPC virus replicates similar to WT. Multicycle replication in A549 cells inoculated with IAV harboring a WT or engineered NS segment (MOI=0.01). Viral titers were determined by plaque assay. Example plaque morphologies are shown. Engineered NS segment integrity over serial passaging was confirmed by RT-PCR. FIG. 14D: Virally delivered sgRNA activates reporter gene expression. A549-CR cells expressing dCas9-VP64 and MS2-p65-HSF1 were inoculated with WT, split-NS or a TRIPCa-NS virus (MOI=0.05) targeting the reporter promoter. Activation of the luciferase reporter was measured over the course of infection. FIG. 14E: Virally delivered sgRNAs activate expression of host genes from the endogenous locus. A549-CR cells were inoculated with TRIPC viruses (MOI=5) targeting the indicated gene, a non-targeting control (NT) or mock. Host gene expression was measured at 8 hpi via RT-qPCR. FIG. 14F: TRIPCa is suitable for fitness-based screening. A pool of 34 TRIPC viruses targeting a collection of 10 potential pro- or antiviral host genes were subject to 4 rounds of selection in A549-CR cells. Viruses present at each stage of selection were quantified by deep-sequencing and normalized sgRNA composition is depicted. Viruses activating proviral genes were enriched, with those >3x enriched colored green, while viruses activating antiviral genes drop out, with those >3x depleted colored red. Graph is representative of mean values for 2 replicate screens.

FIG. 14G: TRIPCa screens are highly reproducible. Comparison of two biological replications shows nearly identical relative enrichment of TRIPC viruses targeting the indicated host genes after 4 rounds of selection. FIG. 14H: TRIPC results reflect changes in viral replication. Multicycle replication in A549-CR cells of TRIPC viruses targeting specific host genes (MOI=0.01). Data are shown as means of 2 (f) or 3 (b-e, h) replicates±SEM (b, d) or s.d. (c, e, h). Pairwise T-tests or one-way ANOVA with post-hoc Tukey's tests were performed (*p<0.05, ** p<0.01, *** p<0.001, **** p<0.0001).

FIG. 15A: TRIPCa functions in NS from primary isolates of IAV and IBV. TRIPCa of a luciferase reporter targeted by sgRNA expressed from transfected CA04 or IBV TRIPC-NS in the presence of the viral polymerase and NP. FIG. 15B: TRIPC-inhibition (TRIPCi) suppresses gene expression. PR8 TRIPC-NS suppressed reporter gene expression when transfected into cells with the viral polymerase, NP, and dCas9-KRAB. FIG. 15C: TRIPC viruses replicate like WT in multiple cell lines. Multicycle replication kinetics of WT, split-NS, or TRIPCa-NS with a non-targeting sgRNA in MDCK and A549-CR cells (MOI=0.01). FIG. 15D: A549-CR cells support TRIPCa. Luciferase reporter expression was measured in A549-CR cells expressing split-NS or TRIPC-NS targeting the reporter promoter. FIG. 15E: TRIPC targeting does not affect replication in cells lacking the CRISPRa machinery. Multicycle replication of TRIPC viruses targeting specified host genes in WT A549 cells inoculated at MOI=0.01. Data are shown as means of 3 replicates=SEM (a-b, d) or s.d. (c, c). Pairwise T-tests or one-way ANOVA with post-hoc Tukey's tests were performed (*p<0.05, ** p<0.01, *** p<0.001, **** p<0.0001).

FIGS. 16A-16G illustrate genome-wide TRIPC screens identify new pro-IAV host factors. FIG. 16A: Experimental design of a genome-wide TRIPC screen in CRISPRa cells. FIG. 16B: TRIPCa selects viruses that replicate at higher levels. Viral titers (left Y-axis) and number of unique TRIPC viruses (right Y-axis) over the course of 5 rounds of selection for replicates A, B and C. FIG. 16C: TRIPCa enriches specific viruses. Stack plot of the abundance of individual TRIPC viruses. Viruses enriched >4-fold at passage 5 are plotted for each replicate. FIG. 16D: Final abundance is independent of starting abundance. Final abundance of individual TRIPC viruses at passage 5 as a function of their abundance at passage 0 for all replicates. Colors represent viruses >4-fold enriched (green) or >4-fold depleted (red) or unchanged (grey). FIG. 16E: Robust ranking aggregation for top hits. MAGeCK gene scores for the top 30 genes in the TRIPC screens. FIG. 16F: High reproducibility of top hits. Venn diagram of genes enriched >4-fold in the 3 screen replicates. FIG. 16G: Enrichment of top hits. Bubble plot depicting positive selection values for all genes in the screen. Bubble size indicates the number of replicate screens in which that gene was detected. Colored dots represent genes >10-fold enriched, with labelled dots representing genes >20-fold enriched. Genes are randomly positions along the X-axis.

FIGS. 17A-17D illustrate characterization of TRIPC library and gene enrichment analysis. FIG. 17A: Experimental workflow for the creation of a genome-wide TRIPC virus library. FIG. 17B: Individual members in the TRIPC library are evenly distributed. Distribution histogram and cumulative frequency plot of members of the TRIPC virus library. FIG. 17C: Population diversity decreases during selection. Shannon's diversity indices of the viral populations across the 3 TRIPC screens. FIG. 17D: High reproducibility of top hits. Venn diagram of TRIPC viruses that were >4-fold enriched at passage 5 across 3 screen replicates. Some viruses target activation of the same gene. GO analysis highlighting the enriched molecular function pathways among the top 100 genes (above). Groupings of high-level gene functions conferred by the top 100 genes (below).

FIGS. 18A-18I illustrates methods to determine the activity of the identified factors employing the 3′-S′ DNA exonuclease TREX1 as an example. FIGS. 18A and 18B: Multiple TRIPC viruses with distinct targeting sequences activate TREX1. A549-CR cells were inoculated at an MOI =1 with viruses targeting different sites in the TREX1 promoter or a non-targeting control. TREX1 expression was measured by RT-qPCR (FIG. 18A) at 10 hpi and western blotting (FIG. 18B) at 12 hpi. FIG. 18C: TREX1 activation enhances viral growth. Multicycle replication of TREX1- or non-targeting TRIPC viruses in A549-CR cells (MOI=0.01). Titers were determined by plaque assay.

FIG. 18D: TRIPC viruses activating TREX1 gain a fitness advantage. A pool of TRIPC viruses were allowed to compete for 48 h during replication in A549-CR cells (pooled MOI=0.05). Relative abundances at the start (input) and end (output) of the infection for each virus are shown for 2 independent replicates. FIG. 18E: TREX1 over-expression increases replication. Multicycle replication of an influenza A reporter virus was performed in WT A549 cells or those stably expressing TREX1 or the catalytic mutant TREX1^{D18N}. FIGS. 18F-18H: TREX1 knockout (KO) reduces viral replication. FIG. 18F: Viral replication was measure at 48 hpi (MOI=0.05) in 3 distinct TREX1-KO clones. Clones were complemented with TREX1 or TREXID18N, where indicated. Values are compared to replication in parental WT A549 cells. FIG. 18G: Multicycle replication in WT A549 cells, TREX1-KO cells, or complemented cell lines. FIG. 18H: Loss of TREX1 decreases viral protein levels. NP protein levels at 24 hpi (above) and titers at 48 hpi (below) in the indicated cells inoculated with PR8 (MOI=0.01). FIG. 18I: TREX1 stimulates replication of multiple primary influenza virus isolates and VSV. Replication of reporter viruses based on CA04 (MOI=0.5), S009 (MOI=0.05), B/Bris (MOI=0.2) at 48 h, and VSV (MOI=0.001) at 24 h Cells are as described in H). Data are shown as means of 3 replicates #SEM (e-g, i) or s.d. (a, c, h-i). Pairwise T-tests or one-way ANOVA with post-hoc Tukey's tests were performed (*p<0.05, ** p<0.01, *** p<0.001, **** p<0.0001, ns=not significant).

FIGS. 19A-19G illustrates methods to determine the activity of the identified factors employing TREX1 as an example. FIGS. 19A-19B: TRIPC viruses do not activate TREX1 in WT A549 cells. TREX1 expression was measured in WT A549 inoculated with TREX1-targeting TRIPC viruses by RT-qPCR (MOI=1, 10 h) (FIG. 19A) and protein expression (MOI=1, 12 h) (FIG. 19B). FIG. 19C: TRIPC viruses have no growth advantage in WT A549 cells. Multicycle replication of TREX1- or non-targeting TRIPC viruses in WT A549 cells (MOI=0.01). Titers determined by plaque assay FIG. 19D: Transient expression of TREX1 boosts viral replication. Multicycle replication of a reporter IAV (MOI=0.05) in A549 cells transfected with GFP-tagged TREX1, TREXID18N or a GFP-alone control. FIG. 19E: Multicycle replication of a reporter IAV (MOI=0.05) in polyclonal TREX1-KO cells. FIG. 19F: TREX1 genotype of knockout cells. Sanger sequencing traces display CRISPR-Cas9 editing at the TREX1 locus for 3 selected knockout clones. Edits compared to the WT genome are shown for 2 homozygous (B6, C8) and 1 heterozygous (G11) clones. FIG. 19G: Western blot demonstrating TREX1 protein levels in WT, clonal KO, and complemented A549 lines utilized throughout. Endogenous TREX1 and recombinant TREX1-V5-2A are indicated. *=non-specific bands. Data are shown as means of 3 replicates±SEM (d-e) or s.d. (a-c). Pairwise T-tests or one-way ANOVA with post-hoc Tukey's tests were performed (*p<0.05, ** p<0.01, *** p<0.001, **** p<0.0001, ns=not significant).

FIGS. 20A-20G illustrate TREX1 moderates DNA sensing to regulate RNA virus replication. FIG. 20A: TREX1 controls sensing of foreign immunogenic DNA. WT and TREX1-KO reporter cells were transfected with indicated amounts of salmon sperm DNA and innate immune activation was measure with an IFN-stimulated response element (ISRE) reporter. Values are normalized to untransfected cells. FIG. 20B: TREX1 regulates activation of endogenous IFN-stimulated genes (ISGs). WT, TREX-KO, or complemented cells were transfected with salmon sperm DNA. ISG expression was measure by RT-qPCR and normalized to untransfected controls. FIG. 20C: Sensing of foreign DNA suppresses IAV replication. Replication of IAV (MOI=0.05) at 24 hpi in WT, TREX1-KO, or complemented cells transfected with salmon sperm DNA or mock treated. FIG. 20D: IAV replicates better in cells lacking DNA sensing. Multicycle replication of IAV (MOI=0.05) in WT or STING-KO A549 cells. FIG. 20E: Chemical activation of the cGAS/STING pathways blunts IAV replication. Multicycle replication of IAV (MOI=0.05) in A549 cells treated with a STING agonist (diABZI) or a DMSO control. FIG. 20F: Activation of the DNA sensing pathway blocks replication of multiple primary influenza virus isolates and VSV A549 cells were treated with diABZI or control and inoculated with reporter viruses based on CA04 (MOI=0.5), S009 (MOI=0.05), B/Bris (MOI=0.2), or VSV (MOI=0.001). Relative replication was measured at 48 hpi for influenza viruses and 24 hpi for VSV. FIG. 20G: The cGAS/STING pathway is not the only DNA sensor regulating infection. Replication of IAV (MOI=0.05) at 48 hpi in WT and STING-KO A549 cells stably expressing TREX1 where indicated. Data are shown as means of 3 replicates±SEM (a, c-g) or s.d. (b). Pairwise T-tests or one-way ANOVA with post-hoc Tukey's tests were performed (*p<0.05, ** p<0.01, *** p<0.001, **** p<0.0001, ns=not significant).

FIGS. 21A-21D illustrate reporter cell line and IAV replication in MAVS-knockout. FIGS. 21A-21B show IFN signaling and RNA sensing remain intact in TREX1-KO cells. ISRE induction in WT and TREX1-KO reporter cells treated with IFNβ (FIG. 21A) or transfected with poly(I:C). ISRE activation is normalized to untreated and mock-transfected cells, respectively. C. Sensing of foreign nucleic acids blocks IAV replication. Replication of IAV (MOI=0.05) on WT A549 cells treated with the indicated nucleic acid ligands. D. Infection in cells lacking RNA sensing for comparison. Multicycle replication of IAV (MOI=0.05) in MAVSKO cells. Data are shown as means of 3 replicates±SEM. One-way ANOVA with post-hoc Tukey's tests were performed (*p<0.05, ** p<0.01, *** p<0.001, **** p<0.0001, ns=not significant).

FIGS. 22A-22C illustrate TREX1 degrades self-DNA released during IAV infection. FIG. 22A: IAV infection releases dsDNA into the cytoplasm. Immunofluorescence staining of WT, TREX1-KO and complemented A549 cells inoculated with PR8 (MOI=1). Blue=DAPI (nucleus), green=viral NP, red=dsDNA. FIG. 22B: mtDNA release into the cytoplasm is exacerbarted in TEK1-KO cells. Cytosolic fractions were prepared from mock- or PR8-infected (MOI=1) cells. mtDNA was detected and quantified by qPCR and shown relative to mock-infected WT cells. FIG. 22C: Cytosolic DNA activates innate immune sensing. Cytosolic extracts were prepared from mock or infected A549 cells and re-introduced into WT or TREX1-KO ISRE reporter cells. Where indicated, extracts were pretreated with nucleases prior to transfection. ISRE activation is normalized to untransfected cells. Data are shown as means of 3 replicates±SEM (c) or s.d. (b). One-way ANOVA with post-hoc Tukey's tests were performed (*p<0.05, ** p<0.01, *** p<0.001, **** p<0.0001, ns=not significant).

FIGS. 23A-23H illustrate TREX1 tempers the anti-viral host response to IAV infection. A-B. TREX1 dampens DNA sensing FIG. 23A: ISRE induction in WT and TREX1-KO reporter cells transfected with nucleic acids from A549 whole cell extracts±nuclease treatments. ISRE induction was normalized to untransfected cells. FIG. 23B: Activation of endogenous ISGs measured by RT-qPCR in TREX-KO or complemented cell lines transfected with self nucleic acids ±nuclease treatments. Induction values are relative to untransfected cells. FIGS. 23C-23D show TREX1 knockout boosts innate immune activation during infection. FIG. 23C: ISRE induction in WT and TREX1-KO reporter cells infected with increasing amounts of IAV. ISRE induction was normalized to uninfected cells. FIG. 23D: Activation of endogenous ISGs measured by RT-qPCR in cell lines infected with PR8 (MOI=0.5). Induction values are relative to uninfected cells. FIG. 23E: Self-DNA sensing antagonizes IAV replication. IAV replication (MOI=0.05) in WT, TREX1-KO, and complemented A549 cells at 48 hpi. Cells were transfected with self nucleic acids and treated with nucleases where indicated. Viral replication values are relative to untreated WT cells. FIGS. 23F-23H: Loss of TREX1 amplifies innate immune responses. FIG. 23F: Gene enrichment analysis of all host genes upregulated >4-fold as gauged by RNA-seq in TREX1-KO and complemented cells 24 hpi with PR8 (MOI=0.5). FIG. 23G: ISG induction in TREX-KO versus complemented cells following infection with PR8. Only ISGs induced >2-fold are shown, with diagonal lines separating ISGs where the induction level differs by at least 50% between cell lines. Gene enrichment analysis is shown for ISGs with higher induction in TREX1-KO cells (lower left) and ISGs with higher induction in complemented cells (lower right). FIG. 23H: Abundance of IAV transcripts in infected TREX1-KO cells relative to infected complemented cells. Values were compiled from 3 RNA-seq experiments with p-values for each gene segment annotated. Data are shown as means of 3 replicates #SEM (A, C, E) or s.d. (B, D). Pairwise T-tests or one-way ANOVA with post-hoc Tukey's tests were performed (*p<0.05. ** p<0.01, *** p<0.001, **** p<0.0001, ns =not significant).

FIGS. 24A-24C illustrate TREX1 modulates host gene expression but does not alter viral polymerase activity. FIG. 24A: Viral polymerase activity is unchanged by TREX1 expression. IAV polymerase activity was measured in a mini-replicon assay in the presence of exogenous GFP-TREX1 or vector control. Data are shown as means of 3 replicates±SEM. Pairwise T-tests tests were performed (ns=not significant). FIG. 24B: TREX1-KO cells exhibit a chronic inflammatory state. Differential expression analysis of RNA-seq data from uninfected TREX1-KO and complemented cells were subject to gene enrichment analysis for all host genes differentially upregulated >4-fold. Significantly enriched biological processes are shown along with their enrichment values. FIG. 24C: Gene enrichment analysis of all ISGs with >2-fold induction in either TREX1-KO cells or complemented cells. Significantly enriched biological processes are shown along with their enrichment values.

FIG. 25 shows the amino acid sequences for the polypeptides of SEQ ID NOS: 74-91 and the corresponding nucleic acid sequences of SEQ ID NOs: 92-110 that encode the polypeptides of SEQ ID NOS: 74-91.

### DETAILED DESCRIPTION

Many approaches to identify host factors regulating infection have relied upon loss-of-function screens, which leaves a large amount of genetic space unexplored and raises the possibility that entirely new classes of viral co-factors have yet to be discovered. CRISPR activation (CRISPRa) and CRISPR inhibition (CRISPRi) may be used to exploit the programmable nature of Cas9 to recruit transcriptional activators or repressor to discrete genomic loci, respectively. CRISPRa and CRISPRi permit both gain- and loss-of-function screens, something not achievable in prior genome-wide surveys of viral host factors.

As disclosed herein, CRISPR-Cas9 technology was adapted to be programmed by the pathogen itself. The pathogen encodes and expresses the targeting RNA that places Cas9 as specific sites in the host genome, termed transcriptional regulation by pathogen-programmed Cas9 (TRPPC). Using the RNA virus influenza virus as an exemplar, TRPPC viruses were shown to modulate host gene expression. Thus, influenza virus can be engineered to specifically and potently modulate expression of discrete host genes. This process can be adapted to any pathogen capable of delivering the targeting RNA. Given that the pathogen expresses essential components of the TRPPC platform, the screen itself only begins during infection, and only in infected cells, which results in the identification of host regulators in the middle-to-late stages of replication.

A pool of TRPPC influenza viruses was prepared targeting the entire genome and a genetic selection was performed allowing all viruses to compete with each other through multiple rounds of replication in human lung cells. Viruses within that population that activated pro-viral factors gained a replicative advantage and came to quickly dominate the viral population. Because the RNA programming Cas9 is encoded in the viral genome, the viruses with an advantage and their host gene targets are easily determined by deep sequencing. Moreover, as this is a fitness-based screen, TRPPC selections identify and inherently rank-order the most potent host regulators of viral replication. In short, the virus itself does the “heavy lifting” to pinpoint the cellular regulators of viral replication.

As an example, 36 host regulators of influenza virus replication whose expression enhances influenza virus replication, that were identified in a genome wide screen are disclosed herein. Several of these host regulators were individually tested for pro-viral properties for influenza virus. In embodiments, the host factor may increase viral yields ˜10-fold, e.g., in human lung cells. Importantly, over-expression of the host factors results in higher levels of virus replication. These are targets to generate cell lines to increase virus yields.

### Definitions

A “vector” or “delivery” vehicle refers to a macromolecule or association of macromolecules that comprises or associates with a polynucleotide or polypeptide, and which can be used to mediate delivery of the polynucleotide or polypeptide to a cell or intercellular space, either in vitro or in vivo. Illustrative vectors include, for example, plasmids, viral vectors, liposomes, nanoparticles, or microparticles and other delivery vehicles. In embodiments, a polynucleotide to be delivered, sometimes referred to as a “target polynucleotide” or “transgene,” may comprise a coding sequence of interest in gene therapy (such as a gene encoding a protein of therapeutic interest), a coding sequence of interest and/or a selectable or detectable marker.

“Transduction,” “transfection,” “transformation” or “transducing” as used herein, are terms referring to a process for the introduction of an exogenous polynucleotide into a host cell leading to expression of the polynucleotide, e.g., the transgene in the cell, and includes the use of recombinant virus to introduce the exogenous polynucleotide to the host cell. Transduction, transfection or transformation of a polynucleotide in a cell may be determined by methods well known to the art including, but not limited to, protein expression (including steady state levels), e.g., by ELISA, flow cytometry and Western blot, measurement of DNA and RNA by hybridization assays, e.g., Northern blots, Southern blots and gel shift mobility assays. Methods used for the introduction of the exogenous polynucleotide include well-known techniques such as viral infection or transfection, lipofection, transformation and electroporation, as well as other non-viral gene delivery techniques. The introduced polynucleotide may be stably or transiently maintained in the host cell.

“Gene delivery” refers to the introduction of an exogenous polynucleotide into a cell for gene transfer, and may encompass targeting, binding, uptake, transport, localization, replicon integration and expression.

“Gene transfer” refers to the introduction of an exogenous polynucleotide into a cell which may encompass targeting, binding, uptake, transport, localization and replicon integration, but is distinct from and does not imply subsequent expression of the gene.

“Gene expression” or “expression” refers to the process of gene transcription, translation, and post-translational modification.

An “infectious” virus or viral particle is one that comprises a polynucleotide component which is capable of delivering into a cell for which the viral species is trophic. The term does not necessarily imply any replication capacity of the virus.

The term “polynucleotide” refers to a polymeric form of nucleotides of any length, including deoxyribonucleotides or ribonucleotides, or analogs thereof. A polynucleotide may comprise modified nucleotides, such as methylated or capped nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment described herein that is a polynucleotide encompasses both the double-stranded form and each of two complementary single-stranded forms known or predicted to make up the double-stranded form.

A “transcriptional regulatory sequence” refers to a genomic region that controls the transcription of a gene or coding sequence to which it is operably linked. Transcriptional regulatory sequences of use generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Heterologous” means derived from a genotypically distinct entity from the entity to which it is compared. For example, a polynucleotide introduced by genetic engineering techniques into a different cell type is a heterologous polynucleotide (and, when expressed, can encode a heterologous polypeptide). Similarly, a transcriptional regulatory element such as a promoter that is removed from its native coding sequence and operably linked to a different coding sequence is a heterologous transcriptional regulatory element.

A “terminator” refers to a polynucleotide sequence that tends to diminish or prevent read-through transcription (i.e., it diminishes or prevent transcription originating on one side of the terminator from continuing through to the other side of the terminator). The degree to which transcription is disrupted is typically a function of the base sequence and/or the length of the terminator sequence. In particular, as is well known in numerous molecular biological systems, particular DNA sequences, generally referred to as “transcriptional termination sequences” are specific sequences that tend to disrupt read-through transcription by RNA polymerase, presumably by causing the RNA polymerase molecule to stop and/or disengage from the DNA being transcribed. Typical example of such sequence-specific terminators include polyadenylation (“polyA”) sequences, e.g., SV40 polyA. In addition to or in place of such sequence-specific terminators, insertions of relatively long DNA sequences between a promoter and a coding region also tend to disrupt transcription of the coding region, generally in proportion to the length of the intervening sequence. This effect presumably arises because there is always some tendency for an RNA polymerase molecule to become disengaged from the DNA being transcribed, and increasing the length of the sequence to be traversed before reaching the coding region would generally increase the likelihood that disengagement would occur before transcription of the coding region was completed or possibly even initiated. Terminators may thus prevent transcription from only one direction (“uni-directional” terminators) or from both directions (“bi-directional” terminators), and may be comprised of sequence-specific termination sequences or sequence-non-specific terminators or both. A variety of such terminator sequences are known in the art, and illustrative uses of such sequences within the context of the present disclosure are provided below.

“Host cells,” “cell lines,” “cell cultures,” “packaging cell line” and other such terms denote higher eukaryotic cells, such as mammalian cells including human cells, useful in the present disclosure, e.g., to produce recombinant virus or recombinant polypeptide. These cells include the progeny of the original cell that was transduced. It is understood that the progeny of a single cell may not necessarily be completely identical (in morphology or in genomic complement) to the original parent cell.

“Recombinant,” as applied to a polynucleotide means that the polynucleotide is the product of various combinations of cloning, restriction and/or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. Promoters include AAV promoters, e.g., P5, P19, P40 and AAV ITR promoters, as well as heterologous promoters.

An “expression vector” is a vector comprising a region which encodes a gene product of interest, and is used for effecting the expression of the gene product in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

The terms “polypeptide” and “protein” are used interchangeably herein to refer to polymers of amino acids of any length. The terms also encompass an amino acid polymer that has been modified; for example, disulfide bond formation, glycosylation, acetylation, phosphorylation, lipidation, or conjugation with a labeling component.

An “isolated” polynucleotide, e.g., plasmid, virus, polypeptide or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Isolated nucleic acid, peptide or polypeptide is present in a form or setting that is different from that in which it is found in nature. For example, a given DNA sequence (e g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded). Enrichment can be measured on an absolute basis, such as weight per volume of solution, or it can be measured in relation to a second, potentially interfering substance present in the source mixture. For example, a 2-fold enrichment, 10-fold enrichment, 100-fold enrichment, or a 1000-fold enrichment.

A “transcriptional regulatory sequence” refers to a genomic region that controls the transcription of a gene or coding sequence to which it is operably linked. Transcriptional regulatory sequences of use generally include at least one transcriptional promoter and may also include one or more enhancers and/or terminators of transcription.

“Operably linked” refers to an arrangement of two or more components, wherein the components so described are in a relationship permitting them to function in a coordinated manner. By way of illustration, a transcriptional regulatory sequence or a promoter is operably linked to a coding sequence if the TRS or promoter promotes transcription of the coding sequence. An operably linked TRS is generally joined in cis with the coding sequence, but it is not necessarily directly adjacent to it.

“Conservative” amino acid substitutions are, for example, aspartic-glutamic as polar acidic amino acids; lysine/arginine/histidine as polar basic amino acids; leucine/isoleucine/methionine/valine/alanine/glycine/proline as non-polar or hydrophobic amino acids; serine/threonine as polar or uncharged hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide. Naturally occurring residues are divided into groups based on common side-chain properties: (1) hydrophobic: norleucine, met, ala, val, leu, ile; (2) neutral hydrophilic: cys, ser, thr; (3) acidic: asp, glu; (4) basic: asn, gln, his, lys, arg; (5) residues that influence chain orientation: gly, pro, and (6) aromatic; trp, tyr, phe.

The disclosure also envisions polypeptides with non-conservative substitutions. Non-conservative substitutions entail exchanging a member of one of the classes described above for another.

As used herein, “individual” (as in the subject of the treatment) means a mammal. Mammals include, for example, humans; non-human primates, e.g., apes and monkeys; and non-primates, e.g., dogs, cats, rats, mice, cattle, horses, sheep, and goats. Non-mammals include, for example, fish and birds.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, “inhibiting” means inhibition of further progression or worsening of the symptoms associated with the disorder or disease, and “preventing” refers to prevention of the symptoms associated with the disorder or disease.

As used herein, an “effective amount” or a “therapeutically effective amount” of an agent, refers to an amount of the agent that alleviates, in whole or in part, symptoms associated with the disorder or condition, or halts or slows further progression or worsening of those symptoms, or prevents or provides prophylaxis for the disorder or condition, e.g., an amount that is effective to prevent, inhibit or treat in the individual one or more symptoms.

In particular, a “therapeutically effective amount” refers to an amount effective, at dosages and for periods of time necessary, to achieve the desired therapeutic result A therapeutically effective amount is also one in which any toxic or detrimental effects of the agent(s)are outweighed by the therapeutically beneficial effects.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), e.g., between about 100 and 1,000 nucleotides in length (or any integer therebetween), e.g., between about 200 and 500 nucleotides in length. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (e.g., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature, e.g., an expression cassette which links a promoter from one gene to an open reading frame for a gene product from a different gene.

“Transformed” or “transgenic” is used herein to include any host cell or cell line, which has been altered or augmented by the presence of at least one recombinant DNA sequence. The host cells are typically produced by transfection with a DNA sequence in a plasmid expression vector, as an isolated linear DNA sequence, or infection with a recombinant viral vector.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, or 6 bases or less or 2 bases or less. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less or 2 or less. Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof are more homologous if their amino acids are greater than or equal to 50% identical when optimally aligned using the ALIGN program.

The term “corresponds to” is used herein to mean that a polynucleotide sequence is structurally related to all or a portion of a reference polynucleotide sequence, or that a polypeptide sequence is structurally related to all or a portion of a reference polypeptide sequence, e.g., they have at least 80%, 82%, 85%, 87%, 90%, 92%, 95%, 97% or more, e.g., 99% or 100%, sequence identity. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A. T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, e.g., at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

As used herein, “substantially pure” or “purified” means an object species is the predominant species present (i.e., on a molar basis it is more abundant than any other individual species in the composition), for instance, a substantially purified fraction is a composition wherein the object species comprises at least about 50 percent (on a molar basis) of all macromolecular species present. Generally, a substantially pure composition will comprise more than about 80 percent of all macromolecular species present in the composition, or more than about 85%, about 90%, about 95%, and about 99%. The object species may be purified to essential homogeneity (contaminant species cannot be detected in the composition by conventional detection methods) wherein the composition consists essentially of a single macromolecular species.

### Preparation of Expression Cassettes

To prepare expression cassettes encoding one of SEQ ID Nos. 1-36 or 74-91 or truncated forms thereof (a “portion”), a peptide thereof, or a fusion thereof, for transformation, the recombinant DNA sequence or segment may be circular or linear, double-stranded or single-stranded. A DNA sequence which encodes an RNA sequence that is substantially complementary to a mRNA sequence encoding a gene product of interest is typically a “sense” DNA sequence cloned into a cassette in the opposite orientation (i.e., 3′ to 5′ rather than 5′ to 3′). Generally, the DNA sequence or segment is in the form of chimeric DNA, such as plasmid DNA, that can also contain coding regions flanked by control sequences which promote the expression of the DNA in a cell. As used herein, “chimeric” means that a vector comprises DNA from at least two different species, or comprises DNA from the same species, which is linked or associated in a manner which does not occur in the “native” or wild-type of the species.

Aside from DNA sequences that serve as transcription units, or portions thereof, a portion of the DNA may be untranscribed, serving a regulatory or a structural function. For example, the DNA may itself comprise a promoter that is active in eukaryotic cells, e.g., mammalian cells, or in certain cell types, or may utilize a promoter already present in the genome that is the transformation target of the lymphotrophic virus. Such promoters include the CMV promoter, as well as the SV40 late promoter and retroviral LTRs (long terminal repeat elements), although many other promoter elements well known to the art may be employed, e.g., the MMTV, RSV, MLV or HIV LTR. In embodiments, expression is inducible. In embodiments, a tissue-specific promoter (or enhancer) is employed.

Other elements functional in the host cells, such as introns, enhancers, polyadenylation sequences and the like, may also be a part of the recombinant DNA. Such elements may or may not be necessary for the function of the DNA but may provide improved expression of the DNA by affecting transcription, stability of the mRNA, or the like. Such elements may be included in the DNA as desired to obtain the optimal performance of the transforming DNA in the cell. The recombinant DNA to be introduced into the cells may contain either a selectable marker gene or a reporter gene or both to facilitate identification and selection of transformed cells from the population of cells sought to be transformed. Alternatively, the selectable marker may be carried on a separate piece of DNA and used in a co-transformation procedure. Both selectable markers and reporter genes may be flanked with appropriate regulatory sequences to enable expression in the host cells. Useful selectable markers are well known in the art and include, for example, antibiotic and herbicide-resistance genes, such as neo, hpt, dhfr, bar, aroA, puro, hyg, dapA and the like. See also, the genes listed on Table 1 of Lundquist et. al. (U.S. Pat. No. 5,848,956).

Reporter genes are used for identifying potentially transformed cells and for evaluating the functionality of regulatory sequences. Reporter genes which encode for easily assayable proteins are well known in the art. In general, a reporter gene is a gene which is not present in or expressed by the recipient organism or tissue and which encodes a protein whose expression is manifested by some easily detectable property, e.g., enzymatic activity. Example reporter genes include the chloramphenicol acetyl transferase gene (cat) from Tn9 of E. coli, the beta-glucuronidase gene (gus) of the uidA locus of E. coli, the green, red, or blue fluorescent protein gene, and the luciferase gene. Expression of the reporter gene is assayed at a suitable time after the DNA has been introduced into the recipient cells.

The general methods for constructing recombinant DNA which can transform target cells are well known to those skilled in the art, and the same compositions and methods of construction may be utilized to produce the DNA useful herein.

The recombinant DNA can be readily introduced into the host cells, e.g., mammalian, bacterial, yeast or insect cells, or prokaryotic cells, by transfection with an expression vector comprising the recombinant DNA by any procedure useful for the introduction into a particular cell, e.g., physical or biological methods, to yield a transformed (transgenic) cell having the recombinant DNA so that the DNA sequence of interest is expressed by the host cell. In embodiments, the recombinant DNA is stably integrated into the genome of the cell.

Physical methods to introduce a recombinant DNA into a host cell include calcium-mediated methods, lipofection, particle bombardment, microinjection, electroporation, and the like. Biological methods to introduce the DNA of interest into a host cell include the use of DNA and RNA viral vectors. Viral vectors, e.g., retroviral or lentiviral vectors, have become a widely used method for inserting genes into eukaryotic cells, such as mammalian, e.g., human cells. Other viral vectors can be derived from poxviruses, e.g., vaccinia viruses, herpes viruses, adenoviruses, adeno-associated viruses, baculoviruses, and the like.

To confirm the presence of the recombinant DNA sequence in the host cell, a variety of assays may be performed. Such assays include, for example, molecular biological assays well known to those of skill in the art, such as Southern and Northern blotting, RT-PCR and PCR; biochemical assays, such as detecting the presence or absence of a particular gene product, e.g., by immunological means (ELISAs and Western blots) or by other molecular assays.

To detect and quantitate RNA produced from introduced recombinant DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the recombinant DNA segment in question, they do not provide information as to whether the recombinant DNA segment is being expressed. Expression may be evaluated by specifically identifying the peptide products of the introduced DNA sequences or evaluating the phenotypic changes brought about by the expression of the introduced DNA segment in the host cell.

### Vectors or Vehicles for Delivery

Delivery vectors or vehicles include, for example, viral vectors, microparticles, nanoparticles, liposomes and other lipid-containing complexes, and other macromolecular complexes capable of mediating delivery of a gene or a protein to a host cell, e.g., a gene to provide for recombinant expression of a polypeptide encoded by the gene. Vectors or vehicles can also comprise other components or functionalities that further modulate gene delivery and/or gene expression, or that otherwise provide beneficial properties. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components also might include markers, such as detectable and/or selectable markers that can be used to detect or select for cells that have taken up and are expressing the nucleic acid delivered by the vector or have taken up protein delivered by a vehicle. Such components can be provided as a natural feature of the vector (such as the use of certain viral vectors which have components or functionalities mediating binding and uptake), or vectors can be modified to provide such functionalities. Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., WO 92/08796; and WO 94/28143). Such marker genes can provide an added measure of control that can be advantageous in gene therapy contexts. A large variety of such vectors are known in the art and are generally available.

Vectors or vehicles within the scope of the disclosure include, but are not limited to, isolated nucleic acid, e.g., plasmid-based vectors which may be extrachromosomally maintained, and viral vectors, e.g., recombinant adenovirus, retrovirus, lentivirus, herpesvirus, poxvirus, papilloma virus, or adeno-associated virus, including viral and non-viral vectors, or proteins which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Vectors or vehicles may be administered via any route including, but not limited to, intramuscular, buccal, rectal, intravenous or intracoronary administration, and transfer to cells may be enhanced using electroporation and/or iontophoresis. In embodiments, vectors are locally administered.

In embodiments, an isolated polynucleotide or vector having that polynucleotide, encoding a polypeptide or fusion protein that has substantial identity, e.g., at least 80% or more, e.g., 85%, 87%, 90%, 92%, 95%, 97%, 98%, 99% and up to 100%, amino acid sequence identity to one of SEQ ID NOs. 1-36 or 74-91, or a portion thereof, is envisioned.

### Retroviral Vectors

Retroviral vectors exhibit several distinctive features including their ability to stably and precisely integrate into the host genome providing long-term transgene expression. These vectors can be manipulated ex vivo to eliminate infectious gene particles to minimize the risk of systemic infection and patient-to-patient transmission. Pseudotyped retroviral vectors can alter host cell tropism.

### Lentiviruses

Lentiviruses are derived from a family of retroviruses that include human immunodeficiency virus and feline immunodeficiency virus. However, unlike retroviruses that only infect dividing cells, lentiviruses can infect both dividing and nondividing cells. Although lentiviruses have specific tropisms, pseudotyping the viral envelope with vesicular stomatitis virus yields virus with a broader range (Schnepp et al., Meth. Mol. Med., 69:427 (2002)).

### Adenoviral Vectors

Adenoviral vectors may be rendered replication-incompetent by deleting the early (E1A and E1B) genes responsible for viral gene expression from the genome and are stably maintained into the host cells in an extrachromosomal form. These vectors have the ability to transfect both replicating and nonreplicating cells and, in particular, these vectors have been shown to efficiently infect cardiac myocytes in vivo, e.g., after direction injection or perfusion. Adenoviral vectors have been shown to result in transient expression of therapeutic genes in vivo, peaking at 7 days and lasting approximately 4 weeks. The duration of transgene expression may be improved in systems utilizing neural specific promoters. In addition, adenoviral vectors can be produced at very high titers, allowing efficient gene transfer with small volumes of virus.

### Adeno-Associated Virus Vectors

Recombinant adeno-associated viruses (rAAV) are derived from nonpathogenic parvoviruses, evoke essentially no cellular immune response, and produce transgene expression lasting months in most systems. Moreover, like adenovirus, adeno-associated virus vectors also have the capability to infect replicating and nonreplicating cells and are believed to be nonpathogenic to humans.

AAV vectors include but are not limited to AAV1, AAV2, AAV5, AAV7, AAV8, AAV9 or AAVrh. 10.

### Plasmid DNA Vectors

Plasmid DNA is often referred to as “naked DNA” to indicate the absence of a more elaborate packaging system. Direct injection of plasmid DNA to myocardial cells in vivo has been accomplished. Plasmid-based vectors are relatively nonimmunogenic and nonpathogenic, with the potential to stably integrate in the cellular genome, resulting in long-term gene expression in postmitotic cells in vivo. Plasmid DNA may be delivered to cells as part of a macromolecular complex, e.g., a liposome or DNA-protein complex, and delivery may be enhanced using techniques including electroporation.

### Peptides, Polypeptides and Fusion Proteins

The peptide, polypeptide or fusion proteins can be synthesized in vitro, e.g., by the solid phase peptide synthetic method or by recombinant DNA approaches (see above). The solid phase peptide synthetic method is an established and widely used method. These polypeptides can be further purified by fractionation on immunoaffinity or ion-exchange columns; ethanol precipitation; reverse phase HPLC; chromatography on silica or on an anion-exchange resin such as DEAE; chromatofocusing, SDS-PAGE; ammonium sulfate precipitation; gel filtration using, for example, Sephadex G-75; or ligand affinity chromatography.

Once isolated and characterized, chemically modified derivatives of a given peptide, polypeptide or fusion thereof, can be readily prepared. For example, amides of the peptide, polypeptide or fusion thereof may also be prepared by techniques well known in the art for converting a carboxylic acid group or precursor, to an amide. One method for amide formation at the C-terminal carboxyl group is to cleave the peptide, polypeptide or fusion thereof from a solid support with an appropriate amine, or to cleave in the presence of an alcohol, yielding an ester, followed by aminolysis with the desired amine.

Salts of carboxyl groups of a peptide, polypeptide or fusion thereof may be prepared in the usual manner by contacting the peptide, polypeptide, or fusion thereof with one or more equivalents of a desired base such as, for example, a metallic hydroxide base, e.g., sodium hydroxide; a metal carbonate or bicarbonate base such as, for example, sodium carbonate or sodium bicarbonate; or an amine base such as, for example, triethylamine, triethanolamine, and the like.

N-acyl derivatives of an amino group of the peptide, polypeptide or fusion thereof may be prepared by utilizing an N-acyl protected amino acid for the final condensation, or by acylating a protected or unprotected peptide, polypeptide, or fusion thereof. O-acyl derivatives may be prepared, for example, by acylation of a free hydroxy polypeptide or polypeptide resin. Either acylation may be carried out using standard acylating reagents such as acyl halides, anhydrides, acyl imidazoles, and the like. Both N- and O-acylation may be carried out together, if desired.

Formyl-methionine, pyroglutamine and trimethyl-alanine may be substituted at the N-terminal residue of the polypeptide. Other amino-terminal modifications include aminooxypentane modifications.

In embodiments, an isolated peptide, polypeptide or fusion protein has substantial identity, e.g., at least 80% or more, e.g., 85%, 87%, 90%, 92%, 95%, 97%, 98%, 99% and up to 100%, amino acid sequence identity to one of SEQ ID NOs. 1-36 or 74-91 or portion thereof, is envisioned.

Substitutions may include substitutions which utilize the D rather than L form, as well as other well known amino acid analogs, e.g., unnatural amino acids such as a, a-disubstituted amino acids, N-alkyl amino acids, lactic acid, and the like. These analogs include phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoyl-phenylalanine, phenylglycine, propargylglycine, sarcosine, ε-N,N,N-trimethyllysine, ε-N-acetyllysine, N-acetylserine, N-formylmethionine, 3-methylhistidine, 5-hydroxylysine, w-N-methylarginine, and other similar amino acids and imino acids and tert-butylglycine.

Conservative amino acid substitutions may be employed—that is, for example, aspartic-glutamic as acidic amino acids; lysine/arginine/histidine as polar basic amino acids; leucine/isoleucine/methionine/valine/alanine/proline/glycine non-polar or hydrophobic amino acids; serine/threonine as polar or hydrophilic amino acids Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting peptide, polypeptide or fusion polypeptide. Whether an amino acid change results in a functional peptide, polypeptide or fusion polypeptide can readily be determined by assaying the specific activity of the peptide, polypeptide or fusion polypeptide.

Amino acid substitutions are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic; trp, tyr, phe.

The disclosure also envisions a peptide, polypeptide or fusion polypeptide with non-conservative substitutions. Non-conservative substitutions entail exchanging a member of one of the classes described above for another.

Acid addition salts of the peptide, polypeptide or fusion polypeptide or of amino residues of the peptide, polypeptide or fusion polypeptide may be prepared by contacting the polypeptide or amine with one or more equivalents of the desired inorganic or organic acid, such as, for example, hydrochloric acid. Esters of carboxyl groups of the polypeptides may also be prepared by any of the usual methods known in the art.

### Formulations and Dosages

The polypeptides or fusions thereof, or nucleic acid encoding the polypeptide or fusion or the complement thereof, e.g., RNAi, can be formulated as pharmaceutical compositions and administered to a mammalian host, such as a human patient in a variety of forms adapted to the chosen route of administration, e.g., orally or parenterally, by intravenous, intramuscular, topical or subcutaneous routes.

In embodiments, the polypeptides or fusions thereof, or nucleic acid encoding the polypeptide or fusion, or the complement thereof, may be administered by infusion or injection. Solutions of the polypeptides or fusions thereof, or nucleic acid encoding the polypeptide or fusion or the complement thereof, or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion may include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it may be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active agent in the required amount in the appropriate solvent with various of the other ingredients enumerated above, as required, followed by filter sterilization. In the case of sterile powders for the preparation of sterile injectable solutions, the methods of preparation include vacuum drying and the freeze drying techniques, which yield a powder of the active ingredient plus any additional desired ingredient present in the previously sterile-filtered solutions.

Useful solid carriers may include finely divided solids such as tale, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as antimicrobial agents can be added to optimize the properties for a given use. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the polypeptides or fusions thereof, or nucleic acid encoding the polypeptide or fusion, can be determined by comparing their in vitro activity and in vivo activity in animal models thereof. Methods for the extrapolation of effective dosages in mice, and other animals, to humans are known to the art; for example, see U.S. Pat. No. 4,938,949.

Generally, the concentration of the polypeptides or fusions thereof, or nucleic acid encoding the polypeptide or fusion, or the complement thereof, in a liquid composition, may be from about 0.1-25 wt-%, e.g., from about 0.5-10 wt-%. The concentration in a semi-solid or solid composition such as a gel or a powder may be about 0.1-5 wt-%, e.g., about 0.5-2.5 wt-%.

The amount of the polypeptides or fusions thereof, or nucleic acid encoding the polypeptide or fusion required for use alone or with other agents will vary with the route of administration, the nature of the condition being treated and the age and condition of the patient and will be ultimately at the discretion of the attendant physician or clinician.

The polypeptides or fusions thereof, or nucleic acid encoding the polypeptide or fusion, or the complement thereof, may be conveniently administered in unit dosage form; for example, containing 5 to 1000 mg, conveniently 10 to 750 mg, or conveniently 50 to 500 mg of active ingredient per unit dosage form.

In general, however, a suitable dose may be in the range of from about 0.5 to about 100 mg/kg, e.g., from about 10 to about 75 mg/kg of body weight per day, such as 3 to about 50 mg per kilogram body weight of the recipient per day, for example in the range of 6 to 90 mg/kg/day, e.g., in the range of 15 to 60 mg/kg/day.

### Example Pro-Influenza Virus Host Cell Factors

In embodiments, the pro-viral factor comprises a sodium/hydrogen exchanger 10 isoform 1 (SLC9C1) [Homo sapiens]

(NCBI Reference Sequence NP_898884.1) having
the following amino acid sequence
(SEQ ID NO: 1):
MAGIFKEFFESTEDLPEVILTLSLISSIGAFLNRHLEDFPIPVPVI
LFLLGCSFEVLSFTSSQVQRYANAIQWMSPDLFFRIFTPVVFFTT
AFDMDTYMLQKLFWQILLISIPGFLVNYILVLWHLASVNQLLLKP
TQWLLFSAILVSSDPMLTAAAIRDLGLSRSLISLINGESLMTSVI
SLITFTSIMDEDQRLQSKRNHTLAEEIVGGICSYIIASFLFGILS
SKLIQFWMSTVEGDDVNHISLIFSILYLIFYICELVGMSGIFTLA
IVGLLLNSTSFKAAIEETLLLEFWTFLSRIAFLMVFTFFGLLIPA
HTYLYIEFVDIYYSLNIYLTLIVLRFLTLLLISPVLSRVGHEFSW
RWIFIMVCSEMKGMPNINMALLLAYSDLYEGSDKEKSQILFHGVL
VCLITLVVNRFILPVAVTILGLRDATSTKYKSVCCTFQHFQELTK
SAASALKFDKDLANADWNMIEKAITLENPYMLNEEETTEHQKVKC
PHCNKEIDEIENTEAMELANRRLLSAQIASYQRQYRNEILSQSAV
QVLVGAAESFGEKKGKCMSLDTIKNYSESQKTVTFARKLLLNWVY
NTRKEKEGPSKYFFFRICHTIVFTEEFEHVGYLVILMNIFPFIIS
WISQLNVIYHSELKHTNYCFLTLYILEALLKIAAMRKDFFSHAWN
IFELAITLIGILHVILIEIDTIKYIFNETEVIVFIKVVQFFRILR
IFKLIAPKLLQIIDKRMSHQKTFWYGILKGYVQGEADIMTIIDQI
TSSKQIKQMLLKQVIRNMEHAIKELGYLEYDHPEIAVTVKTKEEI
NVMLNMATEILKAFGLKGIISKTEGAGINKLIMAKKKEVLDSQSI
IRPLTVEEVLYHIPWLDKNKDYINFIQEKAKVVTFDCGNDIFEEG
DEPKGIYIIISGMVKLEKSKPGLGIDQMVESKEKDFPIIDTDYML
SGEIIGEINCLINEPMKYSATCKTVVETCFIPKTHLYDAFEQCSP
LIKQKMWLKLGLAITARKIREHLSYEDWNYNMQLKLSNIYVVDIP
MSTKTDIYDENLIYVILIHGAVEDCLLRKTYRAPFLIPITCHQIQ
SIEDFTKVVIIQTPINMKTFRRNIRKFVPKHKSYLTPGLIGSVGT
LEEGIQEERNVKEDGAHSAATARSPQPCSLIGTKENCKESPRINL
RKVRKE

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a treslin isoform 1 (TICRR) [Homo sapiens]

(NCBI Reference Sequence NP_001294954.1)
having the following amino acid sequence
sequence (SEQ ID NO: 2):
MACCHKVMLL LDTAGGAARH SRVRRAALRL LTYLSCREGL
ARVHWAFKFF DSQGARSRPS RVSDFRELGS RSWEDFEEEL
EARLEDRAHL PGPAPRATHT HGALMETLLD YQWDRPEITS
PTKPILRSSG RRLLDVESEA KEAEAALGGL VNAVFLLAPC
PHSQRELLQF VSGCEAQAQR LPPTPKQVME KLLPKRVREV
MVARKITFYW VDTTEWSKLW ESPDHLGYWT VCELLHHGGG
TVLPSESFSW DFAQAGEMLL RSGIKLSSEP HLSPWISMLP
TDATLNRLLY NSPEYEASFP RMEGMLFLPV EGKEIQETWT
VTLEPLAMHQ RHFQKPVRIF LKGSVAQWSL PTSSTLGTDS
WMLGSPEEST ATQRLLFQQL VSRLTAEELH LVADVDPGEG
RPPITGVISP LSASAMILTV CRTKEAEFQR HVLQTAVADS
PRDTASLESD VVDSILNQTH DSLADTASAA SPVPEWAQQE
LGHTTPWSPA VVEKWFPFCN ISGASSDLME SEGLLQAASA
NKEESSKTEG ELIHCLAELY QRKSREESTI AHQEDSKKKR
GVPRTPVRQK MNTMCRSLKM LNVARLNVKA QKLHPDGSPD
VAGEKGIQKI PSGRTVDKLE DRGRTLRSSK PKDFKTEEEL
LSYIRENYQK TVATGEIMLY ACARNMISTV KMELKSKGTK
ELEVNCLNQV KSSLIKTSKS LRQNLGKKLD KEDKVRECQL
QVFLRLEMCL QCPSINESTD DMEQVVEEVT DLLRMVCLTE
DSAYLAEFLE EILRLYIDSI PKTLGNLYNS LGFVIPQKLA
GVLPTDFFSD DSMTQENKSP LLSVPFLSSA RRSVSGSPES
DELQELRTRS AKKRRKNALI RHKSIAEVSQ NLRQIEIPKV
SKRATKKENS HPAPQQPSQP VKDTVQEVTK VRRNLENQEL
LSPSKRSLKR GLPRSHSVSA VDGLEDKLDN FKKNKGYHKL
LTKSVAETPV HKQISKRLLH RQIKGRSSDP GPDIGVVEES
PEKGDEISLR RSPRIKQLSF SRTHSASFYS VSQPKSRSVQ
RVHSFQQDKS DQRENSPVQS IRSPKSLLFG AMSEMISPSE
KGSARMKKRS RNTLDSEVPA AYQTPKKSHQ KSLSESKTTP
PRISHTPQTP LYTPERLQKS PAKMTPTKQA AFKESIKDSS
SPGHDSPLDS KITPQKRHTQ AGEGTSLETK TPRTPKRQGT
QPPGFLPNCT WPHSVNSSPE SPSCPAPPTS STAQPRRECL
TPIRDPLRTP PRAAAFMGTP QNQTHQQPHV LRAARAEEPA
QKLKDKAIKT PKRPGNSTVT SSPPVTPKKL FTSPLCDVSK
KSPFRKSKIE CPSPGELDQK EPQMSPSVAA SLSCPVPSTP
PELSQRATLD TVPPPPPSKV GKRCRKTSDP RRSIVECQPD
ASATPGVGTA DSPAAPTDSR DDQKGLSLSP QSPPERRGYP
GPGLRSDWHA SSPLLITSDT EHVTLLSEAE HHGIGDLKSN
VLSVEEGEGL RTADAEKSSL SHPGIPPSPP SCGPGSPLMP
SRDVHCTTDG RQCQASAQLD NLPASAWHST DSASPQTYEV
ELEMQASGLP KLRIKKIDPS SSLEAEPLSK EESSLGEESF
LPALSMPRAS RSLSKPEPTY VSPPCPRLSH STPGKSRGQT
YICQACTPTH GPSSTPSPFQ TDGVPWTPSP KHSGKTTPDI
IKDWPRRKRA VGCGAGSSSG RGEVGADLPG SLSLLESEGK
DHGLELSIHR TPILEDFELE GVCQLPDQSP PRNSMPKAEE
ASSWGQFGLS SRKRVLLAKE EADRGAKRIC DLREDSEVSK
SKEGSPSWSA WQLPSTGDEE VEVSGSTPPP SCAVRSCLSA
SALQALTQSP LLFQGKTPSS QSKDPRDEDV DVLPSTVEDS
PFSRAFSRRR PISRTYTRKK IMGTWLEDL

a different isoform of the protein, a polypeptide having the sequence in NP_689472.3, which is incorporated by reference herein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises an olfactory receptor 4C6 (OR4C6) [Homo sapiens]

(NCBI Reference Sequence NP_001004704.1)
having the following amino acid
sequence (SEQ ID NO: 3):
MENQNNVTEF ILLGLIENLE LWKIFSAVEL VMYVATVLEN
LLIVVTIITS QSLRSPMYFF LTFLSLLDVM FSSVVAPKVI
VDTLSKSTTI SLKGCLTQLF VEHFFGGVGI ILLTVMAYDR
YVAICKPLHY TIIMSPRVCC LMVGGAWVGG FMHAMIQLLF
MYQIPFCGPN IIDHFICDLE QLLTLACTDT HILGLLVTLN
SGMMCVAIFL ILIASYTVIL CSLKSYSSKG RHKALSTCSS
HLTVVVLFFV PCIFLYMRPV VTHPIDKAMA VSDSIITPML
NPLIYTLRNA EVKSAMKKLW MKWEALAGK

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a C-type lectin domain family 4 member C isoform 1 (CLEC4C) [Homo sapiens]

(NCBI Reference Sequence NP_001358319.1)
having the following amino acid sequence
(SEQ ID NO: 4):
MVPEEEPQDR EKGLWWFQLK VWSMAVVSIL LLSVCFTVSS
VVPHNEMYSK TVKRLSKLRE YQQYHPSLTC VMEGKDIEDW
SCCPTPWTSF QSSCYFISTG MQSWIKSQKN CSVMGADLVV
INTREEQDFI IQNLKRNSSY FLGLSDPGGR RHWQWVDQTP
YNENVTFWHS GEPNNLDERC AIINFRSSEE WGWNDIHCHV
PQKSICKMKK IYI

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a NADH dehydrogenase [ubiquinone] 1 alpha subcomplex subunit 7(NDUFA7) [Homo sapiens]

(NCBI Reference Sequence: NP_004992.2)
having the following amino acid sequence
(SEQ ID NO: 5):
MASATRLIQR LRNWASGHDL QGKLQLRYQE ISKRTQPPPK
LPVGPSHKLS NNYYCTRDGR RESVPPSIIM SSQKALVSGK
PAESSAVAAT EKKAVTPAPP IKRWELSSDQ PYL

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises an olfactory receptor 51A7 (OR51A7) [Homo sapiens]

(NCBI Reference Sequence: NP_001004749.1)
having the following amino acid
sequence (SEQ ID NO: 6):
MSVLNNSEVK LFLLIGIPGL EHAHIWFSIP ICLMYLLAIM
GNCTILFIIK TEPSLHEPMY YFLAMLAVSD MGLSLSSLPT
MLRVFLFNAM GISPNACFAQ EFFIHGFTVM ESSVLLIMSL
DRFLAIHNPL RYSSILTSNR VAKMGLILAI RSILLVIPFP
FTLRRLKYCQ KNLLSHSYCL HQDTMKLACS DNKINVIYGF
FIALCTMLDL ALIVLSYVLI LKTILSIASL AERLKALNTC
VSHICAVLTF YVPIITLAAM HHFAKHKSPL VVILIADMEL
LVPPLMNPIV YCVKTRQIWE KILGKLLNVC GR

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a chloride channel protein CIC-Kb isoform 1 (CLCNKB) [Homo sapiens]

(NCBI Reference Sequence: NP_000076.2) having
the following amino acid sequence
(SEQ ID NO: 7):
MEEFVGLREG SSGNPVTLQE LWGPCPRIRR GIRGGLEWLK
QKLFRLGEDW YELMTLGVLM ALVSCAMDLA VESVVRAHQW
LYREIGDSHL LRYLSWTVYP VALVSFSSGF SQSITPSSGG
SGIPEVKTML AGVVLEDYLD IKNEGAKVVG LSCTLACGST
LFLGKVGPFV HLSVMMAAYL GRVRTTTIGE PENKSKQNEM
LVAAAAVGVA TVEAAPESGV LFSIEVMSSH FSVWDYWRGF
FAATCGAFMF RLLAVENSEQ ETITSLYKTS FRVDVPEDLP
EIFFFVALGG LCGILGSAYL FCQRIFFGFI RNNRFSSKLL
ATSKPVYSAL ATLVLASITY PPSAGRFLAS RLSMKQHLDS
LEDNHSWALM TQNSSPPWPE ELDPQHLWWE WYHPRFTIFG
TLAFFLVMKF WMLILATTIP MPAGYEMPIF VYGAAIGRLF
GETLSFIFPE GIVAGGITNP IMPGGYALAG AAAFSGAVTH
TISTALLAFE VTGQIVHALP VLMAVLAANA IAQSCQPSFY
DGTVIVKKLP YLPRILGRNI GSHRVRVEHF MNHSITTLAK
DMPLEEVVKV VISTDVAKYP LVESTESQIL VGIVRRAQLV
QALKAEPPSW APGHQQCLQD ILAAGCPTEP VILKLSPETS
LHEAHNLFEL LNLHSLEVTS RGRAVGCVSW VEMKKAISNL
TNPPAPK

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a guanine nucleotide-binding protein G(I)/G(S)/G(O) subunit gamma-5 (GNG5) [Homo sapiens]

(NCBI Reference Sequence: NP_005265.1)
having the following amino
acid sequence (SEQ ID NO: 8):
MSGSSSVAAM KKVVQQLRLE AGLNRVKVSQ AAADLKQFCL
QNAQHDPLLT GVSSSTNPER PQKVCSEL

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a S-adenosyl-L-methionine-dependent tRNA 4-demethylwyosine synthase (TYW1) [Homo sapiens]

(NCBI Reference Sequence: NP_060734.2) having the following amino
acid sequence (SEQ ID NO: 9):
MDPSADTWDL FSPLISLWIN RFYIYLGFAV SISLWICVQI VIKTQGKNLQ EKSVPKAAQD
LMINGYVSLQ EKDIFVSGVK IFYGSQTGTA KGFATVLAEA VTSLDLPVAI INLKEYDPDD
HLIEEVTSKN VCVELVATYT DGLPTESAEW FCKWLEEASI DEREGKTYLK GMRYAVFGLG
NSAYASHENK VGKNVDKWLW MLGAHRVMSR GEGDCDVVKS KHGSIEADER AWKTKFISQL
QALQKGERKK SCGGHCKKGK CESHQHGSEE REEGSHEQDE LHHRDTEEEE PFESSSEEEF
GGEDHQSLNS IVDVEDLGKI MDHVKKEKRE KEQQEEKSGL FRNMGRNEDG ERRAMITPAL
REALTKQGYQ LIGSHSGVKL CRWTKSMLRG RGGCYKHTFY GIESHRCMET TPSLACANKC
VFCWRHHTNP VGTEWRWKMD QPEMILKEAI ENHQNMIKQF KGVPGVKAER FEEGMTVKHC
ALSLVGEPIM YPEINRFLKL LHQCKISSEL VTNAQFPAEI RNLEPVTQLY VSVDASTKDS
LKKIDRPLEK DEWQRFLDSL KALAVKQQRT VYRLTLVKAW NVDELQAYAQ LVSLGNPDFI
EVKGVTYCGE SSASSLTMAH VPWHEEVVQF VHELVDLIPE YEIACEHEHS NCLLIAHRKE
KIGGEWWTWI DYNRFQELIQ EYEDSGGSKT FSAKDYMART PHWALFGASE RGFDPKDTRH
QRKNKSKAIS GC

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a ras-related protein Rab-42 isoform 1 (RAB42) [Homo sapiens]

(NCBI Reference Sequence:
NP_001180461.1) having the following
amino acid sequence (SEQ ID NO: 10):
MEAEGCRYQF RVALLGDAAV GKTSLLRSYV
AGAPGAPEPE PEPEPTVGAE CYRRALQLRA
GPRVKLQLWD TAGHERFRCI TRSFYRNVVG
VLLVEDVINR KSFEHIQDWH QEVMATQGPD
KVIFLLVGHK SDLQSTRCVS AQEAEELAAS
LGMAFVETSV KNNCNVDLAF DTLADAIQQA
LQQGDIKLEE GWGGVRLIHK TQIPRSPSRK
QHSGPCQC

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a potassium/sodium hyperpolarization-activated cyclic nucleotide-gated channel 3 (HCN3) [Homo sapiens]

(NCBI Reference Sequence: NP_065948.1) having the following
amino acid sequence (SEQ ID NO: 11):
MEAEQRPAAG ASEGATPGLE AVPPVAPPPA TAASGPIPKS GPEPKRRHLG TLLQPTVNKE
SLRVFGSHKA VEIEQERVKS AGAWITHPYS DERFYWDLIM LLLMVGNLIV LPVGITFFKE
ENSPPWIVEN VISDIFFLLD LVLNFRTGIV VEEGAEILLA PRAIRTRYLR TWELVDLISS
IPVDYIFLVV ELEPRLDAEV YKTARALRIV RFTKILSLLR LLRLSRLIRY IHQWEEIFHM
TYDLASAVVR IFNLIGMMLL LCHWDGCLQF LVPMLQDFPP DCWVSINHMV NHSWGRQYSH
ALFKAMSHML CIGYGQQAPV GMPDVWLTML SMIVGATCYA MFIGHATALI QSLDSSRRQY
QEKYKQVEQY MSFHKLPADT RQRIHEYYEH RYQGKMEDEE SILGELSEPL REEIINFTCR
GLVAHMPLFA HADPSFVTAV LTKLRFEVFQ PGDLVVREGS VGRKMYFIQH GLLSVLARGA
RDTRLTDGSY FGEICLLTRG RRTASVRADT YCRLYSLSVD HFNAVLEEFP MMRRAFETVA
MDRLLRIGKK NSILQRKRSE PSPGSSGGIM EQHLVQHDRD MARGVRGRAP STGAQLSGKP
VLWEPLVHAP LQAAAVTSNV AIALTHQRGP LPLSPDSPAT LLARSAWRSA GSPASPLVPV
RAGPWASTSR LPAPPARTLH ASLSRAGRSQ VSLLGPPPGG GGRRLGPRGR PLSASQPSLP
QRATGDGSPG RKGSGSERLP PSGLLAKPPR TAQPPRPPVP EPATPRGLQL SANM

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a rasGAP-activating-like protein 1 isoform 1 (RASAL1) [Homo sapiens]

(NCBI Reference Sequence: NP_001180449.1) having the
following amino acid sequence (SEQ ID NO: 12):
MAKSSSLNVR VVEGRALPAK DVSGSSDPYC LVKVDDEVVA RTATVWRSLG PFWGEEYTVH
LPLDFHQLAF YVLDEDTVGH DDIIGKISLS REAITADPRG IDSWINLSRV DPDAEVQGEI
CLSVQMLEDG QGRCLRCHVL QARDLAPRDI SGTSDPFARV FWGSQSLETS TIKKTREPHW
DEVLELREMP GAPSPLRVEL WDWDMVGKND FLGMVEFSPK TLQQKPPKGW FRLLPFPRAE
EDSGGNLGAL RVKVRLIEDR VLPSQCYQPL MELLMESVQG PAEEDTASPL ALLEELTLGD
CRQDLATKLV KLFLGRGLAG RFLDYLTRRE VARTMDPNTL FRSNSLASKS MEQFMKLVGM
PYLHEVLKPV ISRVFEEKKY MELDPCKMDL GRTRRISEKG ALSEEQMRET SLGLLTGYLG
PIVDAIVGSV GRCPPAMRLA FKQLHRRVEE RFPQAEHQQD VKYLAISGFL FLRFFAPAIL
TPKLFDLRDQ HADPQTSRSL LLLAKAVQSI GNLGQQLGQG KELWMAPLHP FLLQCVSRVR
DFLDRLVDVD GDEEAGVPAR ALFPPSAIVR EGYLLKRKEE PAGLATREAF KKRYVWLSGE
TLSFSKSPEW QMCHSIPVSH IRAVERVDEG AFQLPHVMQV VTQDGTGALH TTYLQCKNVN
ELNQWLSALR KASAPNPNKL AACHPGAFRS ARWTCCLQAE RSAAGCSRTH SAVTLGDWSD
PLDPDAEAQT VYRQLLLGRD QLRLKLLEDS NMDTTLEADT GACPEVLARQ RAATARLLEV
LADLDRAHEE FQQQERGKAA LGPLGP

a different isoform of the protein, a polypeptide having the sequence in NP_001288131.1, which is incorporated by reference herein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a UL16-binding protein 1 isoform 1 precursor (ULBP1) [Homo sapiens]

(NCBI Reference Sequence: NP_079494.1) having the
following amino acid sequence (SEQ ID NO: 13):
MAAAASPAFL LCLPLLHLLS GWSRAGWVDT HCLCYDFIIT PKSRPEPQWC EVQGLVDERP
FLHYDCVNHK AKAFASLGKK VNVTKTWEEQ TETLRDVVDF LKGQLLDIQV ENLIPIEPLT
LQARMSCEHE AHGHGRGSWQ FLENGQKELL FDSNNRKWTA LHPGAKKMTE KWEKNRDVTM
FFQKISLGDC KMWLEEFLMY WEQMLDPTKP PSLAPGTTQP KAMATTLSPW SLLIIFLCFI
LAGR

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a macrophage immunometabolism regulator (C5orf30) [Homo sapiens]

(NCBI Reference Sequence: NP_149988.1) having the following
amino acid sequence (SEQ ID NO: 14):
MEVDINGESR STLITLPFPG AEANSPGKAE AEKPRCSSTP CSPMRRTVSG YQILHMDSNY
LVGFTTGEEL LKLAQKCTGG EESKAEAMPS LRSKQLDAGL ARSSRLYKTR SRYYQPYEIP
AVNGRRRRRM PSSGDKCTKS LPYEPYKALH GPLPLCLLKG KRAHSKSLDY LNLDKMIKEP
ADTEVLQYQL QHLTLRGDRV FARNNT

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a protein mono-ADP-ribosyltransferase PARP15 isoform 1 [Homo sapiens]

(NCBI Reference Sequence: NP_001106995.1)
having the following amino acid sequence (SEQ ID NO: 15):
MAAPGPLPAA ALSPGAPTPR ELMHGVAGVT SRAGRDREAG SVIPAGNRGA RKASRRSSSR
SMSRDNKFSK KDCLSIRNVV ASIQTKEGLN LKLISGDVLY IWADVIVNSV PMNLQLGGGP
LSRAFLQKAG PMLQKELDDR RRETEEKVGN IFMTSGCNLD CKAVLHAVAP YWNNGAETSW
QIMANIIKKC LTTVEVLSES SITFPMIGTG SLQFPKAVFA KLILSEVFEY SSSTRPITSP
LQEVHELVYT NDDEGCQAFL DEFINWSRIN PNKARIPMAG DTQGVVGTVS KPCFTAYEMK
IGAITFQVAT GDIATEQVDV IVNSTARTEN RKSGVSRAIL EGAGQAVESE CAVLAAQPHR
DFIITPGGCL KCKIIIHVPG GKDVRKTVTS VLEECEQRKY TSVSLPAIGT GNAGKNPITV
ADNIIDAIVD FSSQHSTPSL KTVKVVIFQP ELLNIFYDSM KKRDLSASIN FQSTFSMTTC
NLPEHWIDMN HQLFCMVQLE PGQSEYNTIK DKFTRTCSSY AIEKIERIQN AFLWQSYQVK
KRQMDIKNDH KNNERLLFHG TDADSVPYVN QHGENRSCAG KNAVSYGKGT YFAVDASYSA
KDTYSKPDSN GRKHMYVVRV LTGVFTKGRA GLVTPPPKNP HNPTDLEDSV INNTRSPKLF
VVFFDNQAYP EYLITFTA

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a neuroligin-4, X-linked (NLGN4X) [Homo sapiens]

MSRPQGLLWL PLLFTPVCVM LNSNVLLWLT ALAIKFTLID SQAQYPVVNT NYGKIRGLRT
PLPNEILGPV EQYLGVPYAS PPTGERRFQP PEPPSSWTGI RNTTQFAAVC PQHLDERSLL
HDMLPIWFTA NLDTLMTYVQ DQNEDCLYLN IYVPTEDDIH DQNSKKPVMV YIHGGSYMEG
TGNMIDGSIL ASYGNVIVIT INYRLGILGF LSTGDQAAKG NYGLLDQIQA LRWIEENVGA
FGGDPKRVTI FGSGAGASCV SLLTLSHYSE GLFQKAIIQS GTALSSWAVN YQPAKYTRIL
ADKVGCNMLD TIDMVECLRN KNYKELIQQT ITPATYHIAF GPVIDGDVIP DDPQILMEQG
EFLNYDIMLG VNQGEGLKFV DGIVDNEDGV TPNDEDFSVS NFVDNLYGYP EGKDTLRETI
KEMYTDWADK ENPETRRKTL VALFTDHQWV APAVATADLH AQYGSPTYFY AFYHHCQSEM
KPSWADSAHG DEVPYVFGIP MIGPTELFSC NFSKNDVMLS AVVMTYWTNF AKTGDPNQPV
PQDTKFIHTK PNRFEEVAWS KYNPKDQLYL HIGLKPRVRD HYRATKVAFW LELVPHLHNL
NEIFQYVSTT TKVPPPDMTS FPYGTRRSPA KIWPTTKRPA ITPANNPKHS KDPHKTGPED
TTVLIETKRD YSTELSVTIA VGASLLELNI LAFAALYYKK DKRRHETHRR PSPQRNITND
IAHIQNEEIM SLQMKQLEHD HECESLQAHD TLRLTCPPDY TLTLRRSPDD IPLMTPNTIT
MIPNTLTGMQ PLHTENTFSG GQNSTNLPHG HSTTRV

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a CD59 glycoprotein preproprotein (CD59) [Homo sapiens]

(NCBI Reference Sequence: NP_000602.1)
having the following amino
acid sequence (SEQ ID NO: 17):
MGIQGGSVLF GLLLVLAVFC HSGHSLQCYN
CPNPTADCKT AVNCSSDFDA CLITKAGLQV
YNKCWKFEHC NENDVTTRLR ENELTYYCCK
KDLCNENEQL ENGGTSLSEK TVLLLVTPFL
AAAWSLHP

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a cofilin-2 isoform 1 (CFL2) [Homo sapiens]

(NCBI Reference Sequence: NP_619579.1)
having the following
amino acid sequence
(SEQ ID NO: 18):
MASGVTVNDE VIKVENDMKV RKSSTQEEIK
KRKKAVLFCL SDDKRQIIVE EAKQILVGDI
GDTVEDPYTS FVKLLPLNDC RYALYDATYE
TKESKKEDLV FIFWAPESAP LKSKMIYASS
KDAIKKKFTG IKHEWQVNGL DDIKDRSTLG
EKLGGNVVVS LEGKPL

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a gasdermin-B isoform 1 (GSDMB) [Homo sapiens]

(NCBI Reference Sequence: NP_001035936.1) having the following amino acid
sequence (SEQ ID NO: 19):
MFSVFEEITR IVVKEMDAGG DMIAVRSLVD ADRFRCFHLV GEKRTFFGCR HYTTGLILMD
ILDTDGDKWL DELDSGLQGQ KAEFQILDNV DSTGELIVRL PKEITISGSF QGFHHQKIKI
SENRISQQYL ATLENRKLKR ELPFSERSIN TRENLYLVTE TLETVKEETL KSDRQYKEWS
QISQGHLSYK HKGQREVTIP PNRVLSYRVK QLVEPNKETM KKDGASSCLG KSLGSEDSRN
MKEKLEDMES VLKDLTEEKR KDVLNSLAKC LGKEDIRQDL EQRVSEVLIS GELHMEDPDK
PLLSSLFNAA GVLVEARAKA ILDFLDALLE LSEEQQFVAE ALEKGTLPLL KDQVKSVMEQ
NWDELASSPP DMDYDPEARI LCALYVVVSI LLELAEGPTS VSS

a different isoform of the protein, a polypeptide having the sequence in NP_001159430.1, which is incorporated by reference herein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a bromodomain-containing protein 4 isoform long (BRD4) [Homo sapiens]

(NCBI Reference Sequence: NP_001366220.1) having the
following amino acid sequence (SEQ ID NO: 20):
MSAESGPGTR LRNLPVMGDG LETSQMSTTQ AQAQPQPANA ASTNPPPPET SNPNKPKRQT
NQLQYLLRVV LKTLWKHQFA WPFQQPVDAV KLNLPDYYKI IKTPMDMGTI KKRLENNYYW
NAQECIQDEN TMFTNCYTYN KPGDDIVLMA EALEKLFLQK INELPTEETE IMIVQAKGRG
RGRKETGTAK PGVSTVPNTT QASTPPQTQT PQPNPPPVQA TPHPFPAVTP DLIVQTPVMT
VVPPQPLQTP PPVPPQPQPP PAPAPQPVQS HPPIIAATPQ PVKTKKGVKR KADTTTPTTI
DPIHEPPSLP PEPKTIKLGQ RRESSRPVKP PKKDVPDSQQ HPAPEKSSKV SEQLKCCSGI
LKEMFAKKHA AYAWPFYKPV DVEALGLADY CDIIKHPMDM STIKSKLEAR EYRDAQEFGA
DVRLMFSNCY KYNPPDHEVV AMARKLQDVE EMRFAKMPDE PEEPVVAVSS PAVPPPTKVV
APPSSSDSSS DSSSDSDSST DDSEEERAQR LAELQEQLKA VHEQLAALSQ PQQNKPKKKE
KDKKEKKKEK HKRKEEVEEN KKSKAKEPPP KKTKKNNSSN SNVSKKEPAP MKSKPPPTYE
SEEEDKCKPM SYEEKRQLSL DINKLPGEKL GRVVHIIQSR EPSLKNSNPD EIEIDFETLK
PSTLRELERY VTSCLRKKRK PQAEKVDVIA GSSKMKGFSS SESESSSESS SSDSEDSETE
MAPKSKKKGH PGREQKKHHH HHHQQMQQAP APVPQQPPPP PQQPPPPPPP QQQQQPPPPP
PPPSMPQQAA PAMKSSPPPF IATQVPVLEP QLPGSVEDPI GHFTQPILHL PQPELPPHLP
QPPEHSTPPH LNQHAVVSPP ALHNALPQQP SRPSNRAAAL PPKPARPPAV SPALTQTPLL
PQPPMAQPPQ VLLEDEEPPA PPLTSMQMQL YLQQLQKVQP PTPLLPSVKV QSQPPPPLPP
PPHPSVQQQL QQQPPPPPPP QPQPPPQQQH QPPPRPVHLQ PMQFSTHIQQ PPPPQGQQPP
HPPPGQQPPP PQPAKPQQVI QHHHSPRHHK SDPYSTGHLR EAPSPLMIHS PQMSQFQSLT
HQSPPQQNVQ PKKQELRAAS VVQPQPLVVV KEEKIHSPII RSEPFSPSLR PEPPKHPESI
KAPVHLPQRP EMKPVDVGRP VIRPPEQNAP PPGAPDKDKQ KQEPKTPVAP KKDLKIKNMG
SWASLVQKHP TTPSSTAKSS SDSFEQFRRA AREKEEREKA LKAQAEHAEK EKERLRQERM
RSREDEDALE QARRAHEEAR RRQEQQQQQR QEQQQQQQQQ AAAVAAAATP QAQSSQPQSM
LDQQRELARK REQERRRREA MAATIDMNFQ SDLLSIFEEN LF

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises an interferon-induced protein with tetratricopeptide repeats 3 isoform a (IFIT3) [Homo sapiens]

(NCBI Reference Sequence:
NP_001540.2) having the following amino acid sequence (SEQ ID NO: 21):
MSEVIKNSLE KILPQLKCHF TWNLFKEDSV SRDLEDRVCN QIEFLNTEFK ATMYNLLAYI
KHLDGNNEAA LECLRQAEEL IQQEHADQAE IRSIVIWGNY AWVYYHLGRL SDAQIYVDKV
KQTCKKESNP YSIEYSELDC EEGWTQLKCG RNERAKVCFE KALEEKPNNP EFSSGLAIAM
YHLDNHPEKQ FSTDVLKQAI ELSPDNQYVK VLLGLKLQKM NKEAEGEQFV EEALEKSPCQ
TDVLRSAAKF YRRKGDLDKA IELFQRVLES TPNNGYLYHQ IGCCYKAKVR QMQNTGESEA
SGNKEMIEAL KQYAMDYSNK ALEKGLNPLN AYSDLAEFLE TECYQTPENK EVPDAEKQQS
HQRYCNLQKY NGKSEDTAVQ HGLEGLSISK KSTDKEEIKD QPQNVSENLL PQNAPNYWYL
QGLIHKQNGD LLQAAKCYEK ELGRLLRDAP SGIGSIFLSA SELEDGSEEM GQGAVSSSPR
ELLSNSEQLN

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises an opioid growth factor receptor (OGFR) [Homo sapiens]

(NCBI Reference Sequence: NP_031372.2) having the following amino
acid sequence (SEQ ID NO: 22):
MDDPDCDSTW EEDEEDAEDA EDEDCEDGEA AGARDADAGD EDEESEEPRA ARPSSFQSRM
TGSRNWRATR DMCRYRHNYP DLVERDCNGD TPNLSFYRNE IRFLPNGCFI EDILQNWTDN
YDLLEDNHSY IQWLFPLREP GVNWHAKPLT LREVEVEKSS QEIQERLVRA YELMLGFYGI
RLEDRGTGTV GRAQNYQKRF QNLNWRSHNN LRITRILKSL GELGLEHFQA PLVRFFLEET
LVRRELPGVR QSALDYEMEA VRCRHQRRQL VHEAWEHFRP RCKFVWGPQD KLRRFKPSSL
PHPLEGSRKV EEEGSPGDPD HEASTQGRTC GPEHSKGGGR VDEGPQPRSV EPQDAGPLER
SQGDEAGGHG EDRPEPLSPK ESKKRKLELS RREQPPTEPG PQSASEVEKI ALNLEGCALS
QGSLRTGTQE VGGQDPGEAV QPCRQPLGAR VADKVRKRRK VDEGAGDSAA VASGGAQTLA
LAGSPAPSGH PKAGHSENGV EEDTEGRTGP KEGTPGSPSE TPGPSPAGPA GDEPAESPSE
TPGPRPAGPA GDEPAESPSE TPGPRPAGPA GDEPAESPSE TPGPSPAGPT RDEPAESPSE
TPGPRPAGPA GDEPAESPSE TPGPRPAGPA GDEPAESPSE TPGPSPAGPT RDEPAKAGEA
AELQDAEVES SAKSGKP

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises an epimerase family protein SDR39U1 isoform 1 (SDR39U1) [Homo sapiens]

(NCBI Reference Sequence: NP_064580.2) having the following
amino acid sequence (SEQ ID NO: 23):
MRVLVGGGTG FIGTALTQLL NARGHEVTLV SRKPGPGRIT WDELAASGLP SCDAAVNLAG
ENILNPLRRW NETFQKEVIG SRLETTQLLA KAITKAPQPP KAWVLVTGVA YYQPSLTAEY
DEDSPGGDFD FFSNLVTKWE AAARLPGDST RQVVVRSGVV LGRGGGAMGH MLLPERLGLG
GPIGSGHQFF PWIHIGDLAG ILTHALEANH VHGVLNGVAP SSATNAEFAQ TLGAALGRRA
FIPLPSAVVQ AVEGRQRAIM LLEGQKVIPQ RTLATGYQYS FPELGAALKE IVA

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a regulating synaptic membrane exocytosis protein 2 isoform a (RIMS2) [Homo sapiens]

(NCBI Reference Sequence: NP_001093587.1) having the following
amino acid sequence (SEQ ID NO: 24):
MSAPVGPRGR LAPIPAASQP PLQPEMPDLS HLTEEERKII LAVMDRQKKE EEKEQSVLKK
LHQQFEMYKE QVKKMGEESQ QQQEQKGDAP TCGICHKTKF ADGCGHNCSY CQTKFCARCG
GRVSLRSNKV MWVCNLCRKQ QEILTKSGAW FYNSGSNTPQ QPDQKVLRGL RNEEAPQEKK
PKLHEQTQFQ GPSGDLSVPA VEKSRSHGLT RQHSIKNGSG VKHHIASDIA SDRKRSPSVS
RDQNRRYDQR EEREEYSQYA TSDTAMPRSP SDYADRRSQH EPQFYEDSDH LSYRDSNRRS
HRHSKEYIVD DEDVESRDEY ERQRREEEYQ SRYRSDPNLA RYPVKPQPYE EQMRIHAEVS
RARHERRHSD VSLANADLED SRISMLRMDR PSRQRSISER RAAMENQRSY SMERTREAQG
PSSYAQRTTN HSPPTPRRSP LPIDRPDLRR TDSLRKQHHL DPSSAVRKTK REKMETMLRN
DSLSSDQSES VRPPPPKPHK SKKGGKMRQI SLSSSEEELA STPEYTSCDD VEIESESVSE
KGDMDYNWLD HTSWHSSEAS PMSLHPVTWQ PSKDGDRLIG RILLNKRLKD GSVPRDSGAM
LGLKVVGGKM TESGRLCAFI TKVKKGSLAD TVGHLRPGDE VLEWNGRLLQ GATFEEVYNI
ILESKPEPQV ELVVSRPIGD IPRIPDSTHA QLESSSSSFE SQKMDRPSIS VTSPMSPGML
RDVPQFLSGQ LSSQSLSRRT TPEVPRVQIK LWEDKVGHQL IVTILGAKDL PSREDGRPRN
PYVKIYFLPD RSDKNKRRTK TVKKTLEPKW NQTFIYSPVH RREFRERMLE ITLWDQARVR
EEESEFLGEI LIELETALLD DEPHWYKLQT HDVSSLPLPH PSPYMPRRQL HGESPERRLQ
RSKRISDSEV SDYDCDDGIG VVSDYRHDGR DLQSSTLSVP EQVMSSNHCS PSGSPHRVDV
IGRTRSWSPS VPPPQSRNVE QGLRGTRTMT GHYNTISRMD RHRVMDDHYS PDRDRDCEAA
DRQPYHRSRS TEQRPLLERT TTRSRSTERP DTNLMRSMPS LMTGRSAPPS PALSRSHPRT
GSVQTSPSST PVAGRRGRQL PQLPPKGTLD RKAGGKKLRS TVQRSTETGL AVEMRNWMTR
QASRESTDGS MNSYSSEGNL IFPGVRLASD SQFSDELDGL GPAQLVGRQT LATPAMGDIQ
VGMMDKKGQL EVEIIRARGL VVKPGSKTLP APYVKVYLLD NGVCIAKKKT KVARKTLEPL
YQQLLSFEES PQGKVLQIIV WGDYGRMDHK SEMGVAQILL DELELSNMVI GWFKLEPPSS
LVDPTLAPLT RRASQSSLES STGPSYSRS

a different isoform of the protein, or a polypeptide having the sequence in NP_001335413.1, which is incorporated by reference herein or a polypeptide having at least 80%, 82%, 85%, 86%, 88%, 90%, 92%, 94%, 95%, 97%, 98% or 99% amino acid sequence identity thereto.

In embodiments, the pro-viral factor comprises a sia-alpha-2,3-Gal-beta-1,4-G1cNAc-R:alpha 2,8-sialyltransferase (ST8SIA3) [Homo sapiens]

(NCBI Reference Sequence: NP_056963.2) having the following
amino acid sequence (SEQ ID NO: 25):
MRNCKMARVA SVLGLVMLSV ALLILSLISY VSLKKENIFT TPKYASPGAP RMYMFHAGER
SQFALKFLDP SFVPITNSLT QELQEKPSKW KENRTAFLHQ RQEILQHVDV IKNFSLTKNS
VRIGQLMHYD YSSHKYVFSI SNNFRSLLPD VSPIMNKHYN ICAVVGNSGI LTGSQCGQEI
DKSDFVFRCN FAPTEAFQRD VGRKTNLTTF NPSILEKYYN NLLTIQDRNN FFLSLKKLDG
AILWIPAFFF HTSATVTRTL VDFFVEHRGQ LKVQLAWPGN IMQHVNRYWK NKHLSPKRLS
TGILMYTLAS AICEEIHLYG FWPFGEDPNT REDLPYHYYD KKGTKFTTKW QESHQLPAEF
QLLYRMHGEG LTKLTLSHCA

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a cyclin-dependent kinase inhibitor 3 isoform 1 (CDKN3) [Homo sapiens]

(NCBI Reference Sequence: NP_005183.2) having the following
amino acid sequence (SEQ ID NO: 26):
MKPPSSIQTS EFDSSDEEPI EDEQTPIHIS WLSLSRVNCS QFLGICALPG CKEKDVRRNV
QKDTEELKSC GIQDIFVECT RGELSKYRVP NLLDLYQQCG IITHHHPIAD GGTPDIASCC
EIMEELTTCL KNYRKTLIHC YGGLGRSCLV AACLLLYLSD TISPEQAIDS LRDLRGSGAI
QTIKQYNYLH EFRDKLAAHL SSRDSQSRSV SR

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a T-cell immunoglobulin and mucin domain-containing protein 4 isoform 1 precursor (TIMD4) [Homo sapiens]

(NCBI Reference Sequence: NP_612388.2) having the following
amino acid sequence (SEQ ID NO: 27):
MSKEPLILWL MIEFWWLYLT PVTSETVVTE VLGHRVTLPC LYSSWSHNSN SMCWGKDQCP
YSGCKEALIR TDGMRVTSRK SAKYRLQGTI PRGDVSLTIL NPSESDSGVY CCRIEVPGWE
NDVKINVREN LQRASTTTHR TATTTTRRTT TTSPTTTRQM TTTPAALPTT VVTTPDLTTG
TPLQMTTIAV FTTANTCISL TPSTLPEEAT GLLTPEPSKE GPILTAESET VLPSDSWSSV
ESTSADTVLL TSKESKVWDL PSTSHVSMWK TSDSVSSPQP GASDTAVPEQ NKTTKTGQMD
GIPMSMKNEM PISQLLMIIA PSLGFVLFAL FVAFLLRGKL METYCSQKHT RLDYIGDSKN
VLNDVQHGRE DEDGLFTL

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a protein SYS1 homolog isoform a (SYS1) [Homo sapiens]

(NCBI Reference Sequence: NP_291020.1) having the following amino
acid sequence (SEQ ID NO: 28):
MAGQFRSYVW DPLLILSQIV LMQTVYYGSL GLWLALVDGL VRSSPSLDQM FDAEILGEST
PPGRLSMMSF ILNALTCALG LLYFIRRGKQ CLDFTVTVHF FHLLGCWFYS SRFPSALTWW
LVQAVCIALM AVIGEYLCMR TELKEIPLNS APKSNV

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises an ubiquitin D (UBD) [Homo sapiens]

(NCBI Reference Sequence: NP_006389.2) having the following
amino acid sequence (SEQ ID NO: 29):
MAPNASCLCV HVRSEEWDLM TFDANPYDSV KKIKEHVRSK TKVPVQDQVL LLGSKILKPR
RSLSSYGIDK EKTIHLTLKV VKPSDEELPL ELVESGDEAK RHLLQVRRSS SVAQVKAMIE
TIKTGIIPETQ IVTCNGKRLE DGKMMADYGI RKGNLLFLAC YCIGG

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a mediator of RNA polymerase II transcription subunit 17 (MED17) [Homo sapiens]

(NCBI Reference Sequence: NP_004259.3)
having the following amino acid
sequence (SEQ ID NO: 30):
MSGVRAVRIS IESACEKQVH EVGLDGTETY LPPLSMSQNL ARLAQRIDES QGSGSEEEEA
AGTEGDAQEW PGAGSSADQD DEEGVVKFQP SLWPWDSVRN NLRSALTEMC VLYDVLSIVR
DKKEMTLDPV SQDALPPKQN PQTLQLISKK KSLAGAAQIL LKGAERLIKS VTENQENKLQ
RDENSELLRL RQHWKLRKVG DKILGDLSYR SAGSLFPHHG TFEVIKNTDL DLDKKIPEDY
CPLDVQIPSD LEGSAYIKVS IQKQAPDIGD LGTVNLFKRP LPKSKPGSPH WQTKLEAAQN
VLLCKEIFAQ LSREAVQIKS QVPHIVVKNQ IISQPFPSLQ LSISLCHSSN DKKSQKFATE
KQCPEDHLYV LEHNLHLLIR EFHKQTLSSI MMPHPASAPF GHKRMRLSGP QAFDKNEINS
LQSSEGLLEK IIKQAKHIFL RSRAAATIDS LASRIEDPQI QAHWSNINDV YESSVKVLIT
SQGYEQICKS IQLQLNIGVE QIRVVHRDGR VITLSYQEQE LQDELLSQMS QHQVHAVQQL
AKVMGWQVLS FSNHVGLGPI ESIGNASAIT VASPSGDYAI SVRNGPESGS KIMVQFPRNQ
CKDLPKSDVL QDNKWSHLRG PFKEVQWNKM EGRNEVYKME LLMSALSPCL L

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a peroxisome biogenesis factor 13 (PEX13) [Homo sapiens]

(NCBI Reference Sequence: NP_002609.1) having the following amino
acid sequence (SEQ ID NO: 31):
MASQPPPPPK PWETRRIPGA GPGPGPGPTF QSADLGPTLM TRPGQPALTR VPPPILPRPS
QQTGSSSVNT FRPAYSSESS GYGAYGNSFY GGYSPYSYGY NGLGYNRLRV DDLPPSREVQ
QAEESSRGAF QSIESIVHAF ASVSMMMDAT FSAVYNSFRA VLDVANHESR LKIHFTKVES
AFALVRTIRY LYRRLQRMLG LRRGSENEDL WAESEGTVAC LGAEDRAATS AKSWPIFLFF
AVILGGPYLI WKLLSTHSDE VIDSINWASG EDDHVVARAE YDFAAVSEEE ISFRAGDMLN
LALKEQQPKV RGWLLASLDG QTTGLIPANY VKILGKRKGR KTVESSKVSK QQQSFTNPTL
TKGATVADSL DEQEAAFESV FVETNKVPVA PDSIGKDGEK QDL

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises an ubiquitin carboxyl-terminal hydrolase 17-like protein 13 (USP17L13) [Homo sapiens]

(NCBI Reference Sequence: NP_001243784.1)
having the following amino acid sequence (SEQ ID NO: 32):
MEEDSLYLGG EWQFNHESKL TSSRLDAAFA EIQRTSLPEK SPLSCETRVD LCDDLVPEAR
QLAPREKLPL SSRRPAAVGA GLQNMGNTCY VNASLQCITY TPPLANYMLS REHSQTCHRH
KGCMLCTMQA HITRALHNPG HVIQPSQALA AGFHRGKQED AHEFLMFTVD AMKKACLPGH
KQVDHPSKDT TLIHQIFGGY WRSQIKCLHC HGISDTEDPY LDIALDIQAA QSVQQALEQL
VKPEELNGEN AYHCGVCLQR APASKILTLH TSAKVLILVL KRESDVTGNK IAKNVQYPEC
LDMQPYMSQQ NTGPLVYVLY AVLVHAGWSC HNGHYFSYVK AQEGQWYKMD DAEVTAASIT
SVLSQQAYVL FYIQKSEWER HSESVSRGRE PRALGAEDTD RRATQGELKR DHPCLQAPEL
DEHLVERATQ ESTLDRWKEL QEQNKTKPEF NVRKVEGTLP PDVLVIHQSK YKCGMKNHHP
EQQSSLINLS SSTPTHQESM NTGTLASLRG RARRSKGKNK HSKRALLVCQ

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a mirror-image polydactyly gene 1 protein isoform 1 (MIPOL1) [Homo sapiens]

(NCBI Reference Sequence: NP_001374996.1) having the
following amino acid sequence (SEQ ID NO: 33):
MENWSKDITH SYLEQETTGI NKSTQPDEQL TMNSEKSMHR KSTELVNEIT CENTEWPGQR
STNFQIISSY PDDESVYCTT EKYNVMEHRH NDMHYECMTP CQVTSDSDKE KTIAFLLKED
DILRTSNKKL QQKLAKEDKE QRKLKFKLEL QEKETEAKIA EKTAALVEEV YFAQKERDEA
VMSRLQLAIE ERDEAIARAK HMEMSLKVLE NINPEENDMT LQELLNRINN ADTGIAIQKN
GAIIVDRIYK TKECKMRITA EEMSALIEER DAALSKCKRL EQELHHVKEQ NQTSANNMRH
LTAENNQERA LKAKLLSMQQ ARETAVQQYK KLEEEIQTLR VYYSLHKSLS QEENLKDQEN
YILSTYEEAL KNRENIVSIT QQQNEELATQ LQQALTERAN MELQLQHARE ASQVANEKVQ
KLERLVDVLR KKVGTGTMRT VI

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a ribokinase isoform 1 (RBKS) [Homo sapiens]

(NCBI Reference Sequence: NP_071411.1)
having the following amino acid sequence
(SEQ ID NO: 34):
MAASGEPQRQ WQEEVAAVVV VGSCMTDLVS LTSRLPKTGE TIHGHKFFIG FGGKGANQCV
QAARLGAMTS MVCKVGKDSF GNDYIENLKQ NDISTEFTYQ TKDAATGTAS IIVNNEGQNI
IVIVAGANLL INTEDLRAAA NVISRAKVMV CQLEITPATS LEALTMARRS GVKTLENPAP
AIADLDPQFY TLSDVFCCNE SEAEILTGLI VGSAADAGEA ALVLLKRGCQ VVIITLGAEG
CVVLSQTEPE PKHIPTEKVK AVDITGAGDS FVGALAFYLA YYPNLSLEDM LNRSNFIAAV
SVQAAGTQSS YPYKKDLPLT LF

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises an ubiquitin carboxyl-terminal hydrolase 17 (USP17L2) [Homo sapiens]

(NCBI Reference Sequence: NP_958804.2) having the following
amino acid sequence (SEQ ID NO: 35):
MEDDSLYLGG EWQFNHESKL TSSRPDAAFA EIQRTSLPEK SPLSSEARVD LCDDLAPVAR
QLAPRKKLPL SSRRPAAVGA GLQNMGNTCY ENASLQCITY TPPLANYMLS REHSQTCQRP
KCCMLCTMQA HITWALHSPG HVIQPSQALA AGFHRGKQED AHEFLMFTVD AMKKACLPGH
KQVDHHSKDT TLIHQIFGGC WRSQIKCLHC HGISDTEDPY LDIALDIQAA QSVKQALEQL
VKPEELNGEN AYHCGLCLQR APASKTLTLH TSAKVLILVL KRFSDVIGNK LAKNVQYPEC
LDMQPYMSQQ NTGPLVYVLY AVLVHAGWSC HDGHYFSYVK AQEGQWYKMD DAKVTACSIT
SVLSQQAYVL FYIQKSEWER HSESVSRGRE PRALGAEDTD RRATQGELKR DHPCLQAPEL
DERLVERATQ ESTLDHWKFP QEQNKTKPEF NVRKVEGTLP PNVLVIHQSK YKCGMKNHHP
EQQSSLINLS STTRTDQESV NTGTLASLQG RTRRSKGKNK HSKRALLVCQ

a different isoform of the protein, or a polypeptide having at least 80%, 82%, 85%, 86%, 88%, 90%, 92%, 94%, 95%, 97%, 98% or 99% amino acid sequence identity thereto.

In embodiments, the pro-viral factor comprises a dystrophin isoform Dp427m (DMD) [Homo sapiens]

(NCBI Reference Sequence: NP_003997.2) having the following amino acid
sequence (SEQ ID NO: 36):
MLWWEEVEDC YEREDVQKKT FTKWVNAQFS KFGKQHIENL FSDLQDGRRL LDLLEGLTGQ
KLPKEKGSTR VHALNNVNKA LRVLQNNNVD LVNIGSTDIV DGNHKLILGL IWNIILHWQV
KNVMKNIMAG LQQTNSEKIL LSWVRQSTRN YPQVNVINFT TSWSDGLALN ALIHSHRPDL
FDWNSVVCQQ SATQRLEHAF NIARYQLGIE KLLDPEDVDT TYPDKKSILM YITSLFQVLP
QQVSIEAIQE VEMLPRPPKV TKEEHFQLHH QMHYSQQITV SLAQGYERTS SPKPRFKSYA
YTQAAYVTTS DPTRSPFPSQ HLEAPEDKSF GSSIMESEVN LDRYQTALEE VLSWLLSAED
TLQAQGEISN DVEVVKDQFH THEGYMMDLT AHQGRVGNIL QLGSKLIGTG KLSEDEETEV
QEQMNLLNSR WECLRVASME KQSNLHRVLM DLQNQKLKEL NDWLIKTEER TRKMEEEPLG
PDLEDLKRQV QQHKVLQEDL EQEQVRVNSL THMVVVVDES SGDHATAALE EQLKVLGDRW
ANICRWTEDR WVLLQDILLK WQRLTEEQCL FSAWLSEKED AVNKIHTTGF KDQNEMLSSL
QKLAVLKADL EKKKQSMGKL YSLKQDLLST LKNKSVTQKT EAWLDNFARC WDNLVQKLEK
STAQISQAVT TTQPSLTQTT VMETVTTVTT REQILVKHAQ EELPPPPPQK KRQITVDSEI
RKRLDVDITE LHSWITRSEA VLQSPEFAIF RKEGNFSDLK EKVNAIEREK AEKFRKLQDA
SRSAQALVEQ MVNEGVNADS IKQASEQLNS RWIEFCQLLS ERLNWLEYQN NIIAFYNQLQ
QLEQMTTTAE NWLKIQPTTP SEPTAIKSQL KICKDEVNRL SDLQPQIERL KIQSIALKEK
GQGPMFLDAD FVAFTNHFKQ VESDVQAREK ELQTIFDTLP PMRYQETMSA IRTWVQQSET
KLSIPQLSVT DYEIMEQRLG ELQALQSSLQ EQQSGLYYLS TIVKEMSKKA PSEISRKYQS
EFEEIEGRWK KLSSQLVEHC QKLEEQMNKL RKIQNHIQTL KKWMAEVDVF LKEEWPALGD
SEILKKQLKQ CRLLVSDIQT IQPSLNSVNE GGQKIKNEAE PEFASRLETE LKELNTQWDH
MCQQVYARKE ALKGGLEKTV SLQKDLSEMH EWMTQAEEEY LERDFEYKTP DELQKAVEEM
KRAKEEAQQK EAKVKLLTES VNSVIAQAPP VAQEALKKEL ETLITNYQWL CTRLNGKCKT
LEEVWACWHE LLSYLEKANK WLNEVEFKLK TTENIPGGAE EISEVLDSLE NLMRHSEDNP
NQIRILAQTL TDGGVMDELI NEELETENSR WRELHEEAVR RQKLLEQSIQ SAQETEKSLH
LIQESLTFID KQLAAYIADK VDAAQMPQEA QKIQSDLISH EISLEEMKKH NQGKEAAQRV
LSQIDVAQKK LQDVSMKERL FQKPANFEQR LQESKMILDE VKMHLPALET KSVEQEVVQS
QLNHCVNLYK SLSEVKSEVE MVIKTGRQIV QKKQTENPKE LDERVTALKL HYNELGAKVT
ERKQQLEKCL KLSRKMRKEM NVLTEWLAAT DMELTKRSAV EGMPSNLDSE VAWGKATQKE
TEKQKVHLKS ITEVGEALKT VLGKKETLVE DKLSLINSNW IAVTSRAEEW LNLLLEYQKH
METFDQNVDH ITKWIIQADT LLDESEKKKP QQKEDVLKRL KAELNDIRPK VDSTRDQAAN
LMANRGDHCR KLVEPQISEL NHRFAAISHR IKTGKASIPL KELEQFNSDI QKLLEPLEAE
IQQGVNLKEE DENKDMNEDN EGTVKELLQR GDNLQQRITD ERKREEIKIK QQLLQTKHNA
LKDLRSQRRK KALEISHQWY QYKRQADDLL KCLDDIEKKL ASLPEPRDER KIKEIDRELQ
KKKEELNAVR RQAEGLSEDG AAMAVEPTQI QLSKRWREIE SKFAQFRRLN FAQIHTVREE
TMMVMTEDMP LEISYVPSTY LTEITHVSQA LLEVEQLLNA PDLCAKDFED LFKQEESLKN
IKDSLQQSSG RIDIIHSKKT AALQSATPVE RVKLQEALSQ LDFQWEKVNK MYKDRQGRED
RSVEKWRRFH YDIKIFNQWL TEAEQFLRKT QIPENWEHAK YKWYLKELQD GIGQRQTVVR
TLNATGEEII QQSSKTDASI LQEKLGSLNL RWQEVCKQLS DRKKRLEEQK NILSEFQRDL
NEFVLWLEEA DNIASIPLEP GKEQQLKEKL EQVKLLVEEL PLRQGILKQL NETGGPVLVS
APISPEEQDK LENKLKQTNL QWIKVSRALP EKQGEIEAQI KDLGQLEKKL EDLEEQLNHL
LLWLSPIRNQ LEIYNQPNQE GPEDVKETEI AVQAKQPDVE EILSKGQHLY KEKPATQPVK
RKLEDLSSEW KAVNRLLQEL RAKQPDLAPG LITIGASPTQ TVTLVTQPVV TKETAISKLE
MPSSLMLEVP ALADFNRAWT ELTDWLSLLD QVIKSQRVMV GDLEDINEMI IKQKATMQDL
EQRRPQLEEL ITAAQNLKNK TSNQEARTII TDRIERIQNQ WDEVQEHLQN RRQQLNEMLK
DSTQWLEAKE EAEQVLGQAR AKLESWKEGP YTVDAIQKKI TETKQLAKDL RQWQTNVDVA
NDLALKLLRD YSADDTRKVH MITENINASW RSIHKRVSER EAALEETHRL LQQFPLDLEK
FLAWLTEAET TANVLQDATR KERLLEDSKG VKELMKQWQD LQGEIEAHTD VYHNLDENSQ
KILRSLEGSD DAVLLQRRLD NMNEKWSELR KKSLNIRSHL EASSDQWKRL HLSLQELLVW
LQLKDDELSR QAPIGGDEPA VQKQNDVHRA FKRELKTKEP VIMSTLETVR IFLTEQPLEG
LEKLYQEPRE LPPEERAQNV TRLLRKQAEE VNTEWEKLNL HSADWQRKID ETLERLRELQ
EATDELDLKL RQAEVIKGSW QPVGDLLIDS LQDHLEKVKA LRGEIAPLKE NVSHVNDLAR
QLTTLGIQLS PYNLSTLEDL NTRWKLLQVA VEDRVRQLHE AHRDFGPASQ HELSTSVQGP
WERAISPNKV PYYINHETQT TCWDHPKMTE LYQSLADINN VRESAYRTAM KLRRLQKALC
LDLLSLSAAC DALDQHNLKQ NDQPMDILQI INCLTTIYDR LEQEHNNLVN VPLCVDMCLN
WLLNVYDTGR TGRIRVLSEK TGIISLCKAH LEDKYRYLFK QVASSTGFCD QRRLGLLLHD
SIQIPRQLGE VASEGGSNIE PSVRSCFQFA NNKPEIEAAL FLDWMRLEPQ SMVWLPVLHR
VAAAETAKHQ AKCNICKECP IIGFRYRSLK HENYDICQSC FFSGRVAKGH KMHYPMVEYC
TPTTSGEDVR DFAKVLKNKF RTKRYFAKHP RMGYLPVQTV LEGDNMETPV TLINFWPVDS
APASSPQLSH DDTHSRIEHY ASRLAEMENS NGSYLNDSIS PNESIDDEHL LIQHYCQSLN
QDSPLSQPRS PAQILISLES EERGELERIL ADLEEENRNL QAEYDRLKQQ HEHKGLSPLP
SPPEMMPTSP QSPRDAELIA EAKLLRQHKG RLEARMQILE DHNKQLESQL HRLRQLLEQP
QAEAKVNGTT VSSPSTSLQR SDSSQPMLLR VVGSQTSDSM GEEDLLSPPQ DTSTGLEEVM
EQLNNSFPSS RGRNTPGKPM REDTM

a different isoform of the protein, or a polypeptide having at least 80%, 82%, 85%, 86%, 88%, 90%, 92%, 94%, 95%, 97%, 98% or 99% amino acid sequence identity thereto.

### Example Host Cell Factors for Inhibition.

In embodiments, the disclosure provides for nucleic acid sequences useful to inhibit transcription or translation of mRNA in a host organism, e.g., useful to inhibit RNA, or to enhance viral production. The nucleic acid sequences encode a polypeptides having at least 80%, 82%, 85%, 86%, 88%, 90%, 92%, 94%, 95%, 97%, 98% or 99% amino acid sequence identity to one of SEQ ID Nos. 1-36 or 74-91.

In embodiments, the nucleic acid inhibits expression of a Homo sapiens solute carrier family 9 member C1 (SLC9C1), transcript variant 1, mRNA having NCBI Reference Sequence:

NM_183061.3 (SEQ ID NO: 37):
gagtttggag caagtaactg tcagtgaggt tgcagttggt ctgggctgtt tggctgtgag
cgaaatagct gccccccact tctcacttgc acaccacggg atactectcc tgaggctccg
gatgattcag atggactgtg aaaaacaaca agatggatga tcatatggag attgcttcta
acataaatct gcataaaaat ttttctgaaa catggctgga atatttaagg agtttttttt
cagtactgag gacctccctg aagtcattct aacattgtct ttgatcagct ccattggagc
atttttgaac cggcacttgg aagactttcc aattcctgtc cctgtgatat tatttttact
tggatgcagt tttgaagtat taagctttac atcttcacag gtccaaagat acgcaaacgc
catacaatgg atgagtccag acttattttt tcgtatattt acaccagtag ttttctttac
tactgcattt gacatggata cgtacatget tcaaaagtta ttttggcaga tacttttaat
ttcaattccc ggctttttgg ttaattatat cttagttctt tggcatctgg catctgtaaa
tcaattactt ttgaagccta cccaatggtt attattttca gctatccttg tgagttcaga
tcccatgcta accgcagctg ctataagaga ccttgggctt tctagaagcc tcatcagttt
aattaatgga gaaagtctga tgacctctgt tatatcatta attacattta ctagtattat
ggattttgac caaagactac aaagtaaaag aaaccatacc ttagctgaag agatcgtggg
tggaatttgt tcatatatta tagcaagttt cttgtttgga attctaagtt caaaactgat
tcaattttgg atgtcaactg tttttggtga tgatgtcaat catataagte tcatcttttc
aattctgtat ctcatctttt atatttgtga gttagttgga atgtcaggaa tatttactct
ggccattgtg ggacttcttt taaattctac aagttttaaa gcagcaattg aagaaacact
tcttcttgaa ttctggactt ttctatcacg tattgctttt ctcatggtgt ttactttett
tggacttcta attcctgcac atacatattt gtatatagaa tttgttgata tatactattc
attaaatatc tacttaacat tgattgtttt aagatttctg accettcttt taataagccc
tyttttgtet cgagttggtc atgagttcag tiggcgctgg atattcataa tggtctgtag
tgaaatgaag gggatgccta atataaacat ggcccttctg cttgcctact ctgatcttta
ttttggatct gacaaagaaa aatctcaaat attatttcat ggagtgttag tatgcctaat
aacccttgtt gtcaatagat ttattttgcc agtggcagtt actatactag gtcttcgtga
tgccacatca acaaaatata aatcggtttg ttgcacattt caacacttte aagagctaac
caagtctgca gectctgccc ttaaatttga caaagatctt gctaatgctg attggaacat
gattgagaaa gcaattacac ttgaaaaccc atacatgttg aacgaagaag aaacaacaga
acatcagaag gtgaaatgtc cacactgtaa caaggaaata gatgagatct ttaacactga
agcaatggag ctggccaaca ggcgtctctt gtcagcacaa atagcaagct accagagaca
atacaggaat gagattctgt cccagagtgc tgtccaggtg ttggttggtg cagcagaaag
ttttggtgag aagaagggaa aatgtatgag tcttgataca ataaagaatt attctgaaag
ccaaaaaaca gttacctttg ctagaaaact actacttaat tgggtgtata ataccagaaa
ggaaaaagag ggcccatcaa aatacttctt ttttcgtata tgccatacaa tagtatttac
tgaggaattt gaacatgttg gataccttgt gatattaatg aatatatttc cctttataat
ctcttggata tcccagttaa atgtaatcta ccacagcgaa ttaaaacaca ctaactactg
ttttcttaca ctttatattc tagaggcact acttaagata gcagcaatga ggaaggactt
tttttcacat gcctggaaca tattcgagtt agcaattaca ttaattggca tcttacatgt
aatacttatt gaaatagaca ccattaagta tatttttaat gagactgaag taatagtctt
tataaaagtt gttcaatttt ttcgtatact acgcattttc aagctcatag caccaaagtt
gctgcaaata atagataaaa gaatgagtca tcagaagacc ttttggtatg gaatactaaa
aggctatgtc caaggcgaag cagacataat gaccataatt gatcagatta caagttctaa
acagattaaa cagatgttat taaagcaagt gataaggaat atggaacatg ctataaaaga
gctaggctac ttagagtatg atcacccaga aattgctgtc actgtgaaaa caaaggaaga
aattaatgtt atgctcaata tcgctacaga aattcttaag gcttttgget taaaaggaat
tattagtaaa actgaaggtg ctggaattaa taagttaatc atggccaaaa agaaagaggt
gcttgattct caatctatta tcaggcctct tactgttgaa gaagttctat atcatattcc
gtggetagat aaaaacaaag attatataaa cttcattcag gaaaaagcca aagttgtaac
atttgattgt ggaaatgata tatttgaaga aggtgatgag cccaaaggaa tctatatcat
tatttcagge atggtaaage ttgaaaaatc aaagccaggt ttagggattg atcaaatggt
ggagtcaaag gagaaagatt ttccgataat tgacacagac tatatgctca gtggagaaat
aataggagag ataaactget taactaatga acctatgaaa tattctgcca cctgcaaaac
tgtagtggag acatgtttta ttcccaaaac tcacttgtat gatgcttttg agcaatgctc
tcctctcatt aaacaaaaaa tgtggctaaa acttggactc gctattacag ccagaaaaat
cagagaacac ttatcttatg aggattggaa ctacaatatg caactaaage tctctaatat
ttatgtagta gatataccaa tgagtaccaa aactgatatt tatgatgaaa atctaatcta
tgttatcctc atacatggag ctgtagaaga ttgtctgtta cgaaaaactt atagagcacc
tttcttaatt cctataacat gccatcagat acaaagtatt gaagatttca caaaagtagt
gattattcaa actccgatta acatgaaaac attcagaagg aatattagaa agtttgttcc
taaacataaa agttatctta caccaggatt aataggttca gttggaacat tggaagaagg
cattcaagaa gaaagaaatg ttaaggagga tggagcacac agtgccgcca ctgccaggag
tccccagcct tgctccctgc tggggacaaa gttcaactgt aaggagtccc ctagaataaa
cctaaggaaa gtcaggaaag agtaagactg ttaagaagac cgaagcatgt attaatgctg
tggctatgag aggectcctg ctgcagaaac acacttccct acatcaagaa ggagtaactt
caggttggat cctgtgtgga tgatcttggt gctaagcaga aaagaaattt ggaccttgaa
accagcagtt caacatatat actttttgca aaatttcctt gatttaaaat atttgttatt
ttaaatatac aaaacatttt agaaaatctt agagtaaatt ttagtcttaa agccagaaaa
taagtttata gccatctaga tattttgcat attgctctta cagcaataat ggtttggttc
actttatgaa aaataaaatg tattaaaata tagtttaaa

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of an Homo sapiens olfactory receptor family 4 subfamily C member 6 (OR4C6), mRNA having NCBI Reference Sequence:

NM_001004704.2 (SEQ ID NO: 38):
catgaaggtg gctgatggtg tgattcaaga ttgaactgga agttcaagga ttctcaactc
tcagctggaa ctcatatcaa cacctgagaa atggaaaatc aaaacaatgt gactgaattc
attcttctgg gtctcacaga gaacctggag ctgtggaaaa tattttctgc tgtgtttctt
gtcatgtatg tagccacagt gctggaaaat ctacttattg tggtaactat tatcacaagt
cagagtctga ggtcacctat gtattttttt cttaccttct tgtccctttt ggatgtcatg
ttctcatctg tcgttgcccc caaggtgatt gtagacaccc tctccaagag cactaccatc
tctctcaaag gctgcctcac ccagctgttt gtggagcatt tctttggtgg tgtggggatc
atcctcctca ctgtgatggc ctatgaccgc tacgtggcca tctgtaagcc cctgcactac
acgatcatca tgagtccacg ggtgtgctgc ctaatggtag gaggggcttg ggtgggggga
tttatgcacg caatgataca acttctcttc atgtatcaaa tacccttctg tggtcctaat
atcatagatc actttatatg tgatttgttt cagttgttga cacttgcctg cacggacacc
cacatcctgg gcctcttagt taccctcaac agtgggatga tgtgtgtggc catctttctt
atcttaattg cgtcctacac ggtcatccta tgctccctga agtcttacag ctctaaaggg
cggcacaaag ccctctctac ctgcagctcc cacctcacgg tggttgtatt gttctttgtc
ccctgtattt tcttgtacat gaggcctgtg gtcactcacc ccatagacaa ggcaatggct
gtgtcagact caatcatcac acccatgtta aatcccttga tctatacact gaggaatgca
gaggtgaaaa gtgccatgaa gaaactctgg atgaaatggg aggctttggc tgggaaataa

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of a Homo sapiens C-type lectin domain family 4 member C (CLEC4C), transcript variant 1, mRNA NCBI Reference Sequence:

NM 130441.3 (SEQ ID NO: 39):
actctgtcac ccaggctgga gtgaagtggt acgattacgg ctcactgcaa tccctgcctc
ccaaattcca gtgattctcg tgcctcagcc tcctgagtag ccgaaattac agacgtgtgc
caccatgctt ggctaatttt ttggattttt agtagagatg gggtttcact atgttggcca
ggctagtctt gaactcctgg cctgaagcaa tccgcccacc tcagcctccc aaagtgctga
gattataggc acgagccact acacctggcc acaaaattct ttaaagaagc caatcccatc
ctccctcaag agccaagggg ccacctcacc ctcttgttac agcagatcct gcctcccaca
gtcaccctgc tcccaagtgc aacctctgtc tgaccctgca tggtgtgcgg tgccctcctg
cctcaggccg cgaagaagga tctaagggct tggcttgttt gaaagaacca caccccgaaa
gtaacatctt tggagaaagt gatacaagag cttctgcacc cacctgatag aggaagtcca
aagggtgtgc gcacacacaa tggtgcctga agaagagcct caagaccgag agaaaggact
ctggtggttc cagttgaagg tctggtccat ggcagtcgta tccatcttgc tcctcagtgt
ctgtttcact gtgagttctg tggtgcctca caattttatg tatagcaaaa ctgtcaagag
gctgtccaag ttacgagagt atcaacagta tcatccaagc ctgacctgcg tcatggaagg
aaaggacata gaagattgga gctgctgccc aaccccttgg acttcatttc agtctagttg
ctactttatt tctactggga tgcaatcttg gactaagagt caaaagaact gttctgtgat
gggggctgat ctggtggtga tcaacaccag ggaagaacag gatttcatca ttcagaatct
gaaaagaaat tcttcttatt ttctggggct gtcagatcca gggggtcggc gacattggca
atgggttgac cagacaccat acaatgaaaa tgtcacattc tggcactcag gtgaacccaa
taaccttgat gagcgttgtg cgataataaa tttccgttct tcagaagaat ggggctggaa
tgacattcac tgtcatgtac ctcagaagtc aatttgcaag atgaagaaga tctacatata
aatgaaatat tctccctgga aatgtgtttg ggttggcatc caccgttgta gaaagctaaa
ttgatttttt aatttatgtg taagttttgt acaaggaatg cccctaaaat gtttcagcag
gctgtcacct attacactta tgatataatc ca

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of an Homo sapiens NADH:ubiquinone oxidoreductase subunit A7 (NDUFA7),

mRNA having the nucleic acid sequence (SEQ ID NO: 40):
agtatcgcgg acggaagatg gcgtccgcca cccgtctcat ccagcggctg cggaactggg
cgtccgggca tgacctgcag gggaagctgc agctacgcta ccaggagatc tccaagcgaa
ctcagcctcc tcccaagctc cctgtgggtc ctagccacaa gctctccaac aattactatt
gcactcgcga tggccgccgg gaatctgtgc ccccttccat catcatgtcg tcgcagaagg
cgctggtgtc aggcaagcca gcagagagct ctgctgtagc tgccactgag aagaaggcgg
tgactccagc tcctcccata aagaggtggg agctgtcctc ggaccagcct tacctgtgac
actgcaccct cacggccacc cgactacttt gcctccttgg atttcctcca gggagaatgt
gacctaattt atgacaaata cgtagagctc aggtatcact tctagtttta ctttaaaaaa
taaaaaaata gagacagagt ctcaccatgt ttcccaggct gatcttgaac tcctggcctc
aagcgatcct cctgccttga cctcccaaag tgctgggatt

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of a Homo sapiens olfactory receptor family 51 subfamily A member 7 (OR51A7), mRNA having NCBI Reference Sequence:

NM_001004749.2 (SEQ ID NO: 41):
ctgagcatct ggttctggta aggttaagga gctataaatc cttttggaaa cctaaatcta
agatccggct aacgagctca tatctccctc attatgtctg ttctcaataa ctccgaagtc
aagcttttcc ttctgattgg gatcccagga ctggaacatg cccacatttg gttctccatc
cccatttgcc tcatgtacct gcttgccatc atgggcaact gcaccattct ctttattata
aagacagagc cctcgcttca tgagcccatg tattatttcc ttgccatgtt ggctgtctct
gacatgggcc tgtccctctc ctcccttcct accatgttga gggtcttctt gttcaatgcc
atgggaattt cacctaatgc ctgctttgct caagaattct tcattcatgg attcactgtc
atggaatcct cagtacttct aattatgtct ttggaccgct ttcttgccat tcacaatccc
ttaagataca gttctatcct cactagcaac agggttgcta aaatgggact tattttagcc
attaggagca ttctcttagt gattccattt cccttcacct taaggagatt aaaatattgt
caaaagaatc ttctttctca ctcatactgt cttcatcagg ataccatgaa gctggcctgc
tctgacaaca agaccaatgt catctatggc ttcttcattg ctctctgtac tatgctggac
ttggcactga ttgttttgtc ttatgtgctg atcttgaaga ctatactcag cattgcatct
ttggcagaga ggcttaaggc cctaaatacc tgtgtctccc acatctgtgc tgtgctcacc
ttctatgtgc ccatcatcac cctggctgcc atgcatcact ttgccaagca caaaagccct
cttgttgtga tccttattgc agatatgttc ttgttggtgc cgccccttat gaaccccatt
gtgtactgtg taaagactcg acaaatctgg gagaagatct tggggaagtt gcttaatgta
tgtgggagat aagaacttga acaattaggt aataaattat caaccagtag gcatttactg
tcatttgcta tgtgcttaat gccatagaag tcactaatga aggactggat gatggaagtg
aaaagctatg tagtgcagaa tttataataa agttgagaat ataactgaac aggatagaaa
aaaaagtcaa gagatatata agatatagag gtttaattaa cattttaagg gaagttgaag
gaaaatactt ctgtgatgga gcagctggat ttgagtcaac ccataaagaa tgaatacaat
ttgggcagat tgagattacc attcagttag tatctattaa aaatacagat gatatacaaa
gtctaatctc atactgtcaa ggaaaggtaa aatagctatg aaggatgctg atctgattac
aatagaaagt gaaatttaac agatagcaag tgatattttg gataaaataa gtgaaccaga
ttttggagca cctaaaaaaa gctttgaaaa gtctaaattc aggaggtgtt ggaatgccaa
tttcttctct acatgataaa tttcatttta agaagagcgt gcctgtaaac atggattgaa
tttgggagag atgaggaagg aagattaaca ggagaaagca caataatcct gctgtgaagt
gatgactcag aataggcagg gagagaacaa gatcatttag ctgctgttgt tatttttctg
ttataaaacc aacatgtaaa ttatggaaaa ttcacaaatt taactaagtg actaaaagat
aattttaaac cccctatggt tttgctgttt agtttttttc tgtgatttag tctttccctg
cgccttaaaa aaaatcagcc cctctaatat gttcttaaaa attgattcct gcaggacacg
acatttggta ccacaataat tttcactaaa atttatattt taaacttttt ttctcatgta
tagaggaaat acatgatgga aaaatcaaaa gagtatacag ttgaaaatac aatttgaagg
ggggcaaaca agattgatat ggcaatctct ctgggattct aaggtaagag tgttgtaaac
agaaaagaaa agcttttcaa aggaactggg gacttgaatg atgggtttga attttgtctt
gaggatttgg cataggtgac tgaat

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of an Homo sapiens chloride voltage-gated channel Kb (CLCNKB), transcript variant 1, mRNA having NCBI Reference Sequence:

NM_000085.5 (SEQ ID NO: 42):
gaggatgttg attgttggaa cacacacctg tccaggtgca ggggagctgg aggctctgtg
agaggagggc cagctcagcc acagcaggag gactgacagg ggcctgatgg aggagtttgt
ggggctgcgt gaaggctcct cagggaaccc tgtgactctg caggagctgt ggggcccctg
tccccgcatc cgccgaggca tccgaggtgg cctggagtgg ctgaagcaga agctcttccg
cctgggcgag gactggtact tcctgatgac cctcggggtg ctcatggccc tggtcagctg
tgccatggac ttggctgttg agagtgtggt ccgagcgcac cagtggctgt acagggagat
tggggacagc cacctgctcc ggtatctctc ctggactgtg taccctgtgg ccctcgtctc
tttctcttca ggcttctctc agagcatcac accctcctct ggaggttctg gaatcccgga
ggtgaagacc atgttggcgg gtgtggtctt ggaggactac ctggatatca agaactttgg
ggccaaagtg gtgggcctct cctgcaccct ggcctgtggc agcaccctct tcctcgggaa
agtgggccct ttcgtgcacc tgtctgtgat gatggctgcc tacctgggcc gtgtgcgcac
cacgaccatc ggggagcctg agaacaagag caagcaaaac gaaatgctgg tggcagcggc
ggcagtgggc gtggccacag tctttgcagc tcccttcagc ggcgtcctgt tcagcatcga
ggtcatgtct tcccacttct ctgtctggga ttactggagg ggcttctttg cggccacctg
cggggccttc atgttccggc tcctggcggt cttcaacagc gagcaggaga ccatcacctc
cctctacaag accagtttcc gggtggacgt tcccttcgac ctgcctgaga tcttcttttt
tgtggcgctg gggggtctct gtggcatcct gggcagcgct tacctcttct gtcagcgaat
cttctttggc ttcatcagga acaataggtt cagctccaaa ctgctggcca ccagcaagcc
tgtgtactcc gctctggcca ccttggttct cgcctccatc acctacccac ccagcgccgg
ccgcttccta gcttctcggc tgtccatgaa gcagcatctg gactcgctgt tcgacaacca
ctcctgggcg ctgatgaccc agaactccag cccaccctgg cccgaggagc tcgaccccca
gcacctgtgg tgggaatggt accacccgcg gttcaccatc tttgggaccc ttgccttctt
cctggttatg aagttctgga tgctgattct ggccaccacc atccccatgc ctgccgggta
cttcatgccc atctttgtct atggagctgc tatcgggcgc ctctttgggg agactctctc
ttttatcttc cctgagggca tcgtggctgg agggatcacc aatcccatca tgccaggggg
gtatgctctg gcaggggctg cagccttctc aggggctgtg acccacacca tctccacggc
gctgctggcc ttcgaggtga ccggccagat agtgcatgca ctgcccgtgc tgatggcggt
gctggcagcc aacgccattg cacagagctg ccagccctcc ttctatgatg gcaccgtcat
tgtcaagaag ctgccatacc tgccacggat tctgggccgc aacatcggtt cccaccgcgt
gagggtggag cacttcatga accacagcat caccacactg gccaaggaca tgccactgga
ggaggtggtc aaggttgtga cctccacaga cgtggccaag tatcccctgg tggagagcac
agagtcccag atcctggtgg gcatagtgcg aagggcccag ctggtgcagg ccctgaaggc
tgagcctcct tcctgggctc ctggacacca gcagtgtctc caggacatct tggctgcagg
ctgccccaca gaaccagtga ccctgaagct gtccccagag acttccctgc atgaggcaca
caacctcttt gagctgttga accttcattc cctctttgtg acgtcgcggg gcagagctgt
gggctgcgtg tcctgggtgg agatgaagaa agcaatttcc aacctgacaa atccgccagc
cccaaagtga gccggcccag caagatgaaa cagggcaccc cagctgacct ggtactgagg
ttgggctgag accctgcttc tcttccccca tcaccacctg cccctccctc cagcccagct
ccattctttg gcataacagg caactttaac ctagcccaga agaggatggc tcatcctggg
tgggacgatg gctcctgcct tgaaagacaa aaatcccacc ttgggcagag ctgagtgtga
gaagatggaa aaccagtatc tgccagttgc tcagtgactg gccatcacat taatgaatga
tgagattgga gtacactgtc accaagggca ggcacagatg ccttctgggg ttgtctggtt
cccagtgaga ggctcctgag aaaaataaag ctggttccca ga

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of an Homo sapiens G protein subunit gamma 5 (GNG5), mRNA having NCBI Reference Sequence:

NM_005274.3 (SEQ ID NO: 43):
ctcacttccc tcaacccttc ccacaaactg ggaggaaaac tgagacctcc tggtcacccg
ccgccgggcc ttttagaaac tcccacaagc tctgccttcc ctccctggtc ctcttcagac
cccctcttag ttcttcgcgg ctaacggctc gcgctcgggg ccgggtgtgg agctggaaca
gagggctggc aaggcgcgca tgcgcaccga gggtggagcc gctgagcaca gaaccggaaa
cttagagaca aagttcggag ccccgccccc gccgcgcgcc gctgagttgt ctggccccgc
cgacccacgg cccacgaccc accgacccac gaatcggccc ggccgtcgcg tgcaccatgt
ctggctcctc cagcgtcgcc gctatgaaga aagtggttca acagctccgg ctggaggccg
gactcaaccg cgtaaaagtt tcccaggcag ctgcagactt gaaacagttc tgtctgcaga
atgctcaaca tgaccctctg ctgactggag tatcttcaag tacaaatccc ttcagacccc
agaaagtctg ttcctttttg tagtaaaatg aatctttcaa aggtttccca aaccactcct
tatgatccag tgaatattca agagagctac atttgaagcc tgtacaaaag cttatccctg
taacacatgt gccataatat acaaacttct actttcgtca gtccttaaca tctacctctc
tgaattttca tgaatttcta tttcacaagg gtaattgttt tatatacact ggcagcagca
tacaataaaa cttagtatga aacttt

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of an Homo sapiens tRNA-yW synthesizing protein 1 homolog (TYW1), transcript variant 1, mRNA having NCBI Reference Sequence:

NM_018264.4 (SEQ ID NO: 44):
ctggcagtgt catggctgcc cacaggtctg caggcactcg gtacgccgct aacgcggcga
ggtagctcgg tgcgtctcgc ggtaccagtg cgaatcatcg ggctatccag gtccgagatc
ctagtctcct gtcggctctg aggaggatgg atccttctgc ggatacatgg gacctcttct
cacctttaat atcattatgg ataaacaggt tttacattta tttgggcttt gctgttagca
ttagcctttg gatttgtgtc cagattgtca tcaagacgca gggcaagaac ttacaggaaa
aatctgttcc aaaagcagct caggatttga tgacaaatgg ttatgtctcc cttcaagaga
aagacatctt tgtgtctgga gtgaagattt tttatggttc tcagactgga acagcgaagg
gattcgcaac agttcttgct gaagcagtta catccctgga tctgcctgtg gccattatta
atctaaaaga atatgatcca gatgatcatc tgatagaaga ggtgactagt aaaaatgtct
gtgtcttcct ggttgcgaca tacactgacg gcctaccaac tgaaagtgca gagtggttct
gcaaatggtt agaggaagca tccattgatt ttcgatttgg caaaacttac ctgaagggta
tgagatatgc ggtatttggc ctgggaaatt ctgcctatgc tagccacttc aacaaggttg
gcaaaaatgt tgacaagtgg ctctggatgc ttggcgcgca tcgtgtgatg agtcgagggg
agggcgactg cgacgtggtt aaaagcaagc acggcagcat tgaggccgac ttcagagcat
ggaagaccaa gttcatctcc cagctgcagg cacttcagaa aggggagaga aagaagtcct
gtggcggcca ctgcaagaaa ggcaaatgtg aatctcacca acatggctca gaggagaggg
aggaaggatc tcatgagcag gatgaattgc atcatagaga caccgaggag gaagaaccct
ttgagagctc cagtgaagaa gagtttggtg gtgaggacca tcagagccta aattccattg
ttgatgttga agatttgggc aaaattatgg atcatgtgaa gaaagaaaag agagaaaagg
aacagcagga agagaagtct ggtttgttca ggaacatggg gaggaatgaa gatggtgaaa
gaagagctat gataactcct gctctccgag aagcccttac taaacaaggt tatcagttga
ttgggagcca ctcgggggtg aagctttgca ggtggacaaa gtccatgctc cgagggagag
gaggttgtta caaacacaca ttctatggaa ttgagagcca tcgctgcatg gaaaccaccc
cgagcttggc gtgtgctaat aaatgtgtct tctgttggcg gcaccacacc aaccccgtgg
gcactgagtg gcggtggaag atggaccagc ctgaaatgat cttgaaggaa gccattgaaa
accatcagaa catgattaag cagtttaaag gagtaccggg cgtcaaagca gaacgctttg
aagaaggaat gacggtaaag cactgtgcat tgtccctcgt gggagaacca ataatgtacc
cagagatcaa caggtttttg aagctactcc accagtgtaa aatttccagc ttcctggtca
caaacgcaca atttcctgcg gaaatcagga acctcgagcc ggttactcag ctgtatgtca
gtgtggatgc cagtaccaaa gacagcctga agaaaatcga ccgcccactc ttcaaggatt
tctggcagag attccttgac agtttaaaag ccttggcagt caagcaacaa cgaactgtct
acagactgac gctcgtgaaa gcatggaacg tggacgagct ccaggcctac gcgcagctcg
tgtccctggg gaatcctgac ttcatcgaag tgaagggcgt tacctactgc ggagaaagtt
cagcaagcag tcttaccatg gcccacgtgc cctggcatga ggaagtggta cagtttgtcc
acgagttggt ggatctgatc cccgaatatg aaattgcatg tgaacacgaa cactctaatt
gcctcctgat agcacacaga aagtttaaaa ttggtggtga atggtggaca tggatcgatt
ataaccgctt ccaggagctc atccaggaat atgaagatag tggtggatca aaaacgttca
gcgcaaagga ttatatggcc agaactcctc actgggcatt atttggtgcc agtgaaagag
gctttgatcc caaggacaca agacatcaga gaaagaacaa atcaaaggct atttctggat
gttgagatta tctgatttca aggtactgaa ggacaaaaac ttggatggcc tcaaaaggtt
cttgaacacc actgtgattc tccaaggacg aattacgtaa attatacttt catacaaagg
agacgataag gcagtaaaca tggagacacg ggggacagcg tccacactca gagggcctgg
gccacagccc cgatgtttct tttcagaact cagccccttt cctgatttta cttctaagag
gaaaattatt ttggggagga actacacagt cgtgattaga atttatctga tggttttgta
ttataacttg taagacctgc cagaatgcta gtcccgagag tgtcagacaa ggaagaagtc
cctgggactc ttccccttac ccggccctta gatttcatgg agcagccact tagcattgaa
ttgcactacc ctgagctaaa cgtgtctgtg ctttctaaga taagagcttg atccctttct
tctatcttaa gacagcacct cctgaaaaga atcgaagttg tcacaactct caattatttt
ttaaatactg catagattga gttttggttt attaccaacc cttcccagaa ttgcgttgga
tctaaaacta ctagatctca tcccattccc atgtaaatta ccacagaccg cagtaccggg
gctggagcgg agtgaagctg tctgctgtaa gaggagtggc catgtgaggg catggagtca
ttagtctcac aaacacactt tggactgaag aggatcattt ctttttgttc gtgaggtcac
tgtccaggcc tctcatatca tgaccagacg gcgggtctcc atcttctttc actcctgtgg
ccctggctgc tttacacaat ctgttctata aggttcaggt gttttcaagt tggaaagatc
ataaatactc aaaattgttt tcaagttagc aagttctttt aacagtcttt tatgcaaaaa
ttgaattaat aaaataatct tttgtaaaga

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of an Homo sapiens RAB42, member RAS oncogene family (RAB42), transcript variant 1, mRNA having NCBI Reference Sequence:

NM_001193532.3 (SEQ ID NO: 45):
ctagtttagt ccctttatcc tgtgaagtag gggtcatcat tagccccctt ttacagagga
gagaattgag gcttcgagag agagaaactt ggccaggagt ttccactcgg tccgacgccc
tcggtgcccc gccgggtacg gtggctgggc gcggggagcg gggggcggcg ccggcggggc
cccgggcagg ggcggggtcg gggcgcggac aaaaccgccg cggggcggcg gggtggcgga
cgcggccatg gaggccgagg gctgccgcta ccaatttcgg gtcgcgctgc tgggggacgc
ggcggtgggc aagacgtcgc tgctgcggag ctacgtggca ggcgcgcctg gcgccccgga
gccggagccc gagcccgagc ccacggtggg cgccgagtgc taccgccgcg cgctgcagct
gcgggccggg ccgcgggtca agctgcaact ctgggacacc gcgggccacg agcgcttcag
gtgcatcacc aggtcctttt accggaatgt ggtgggtgtc ctgctggtct ttgatgtgac
aaacaggaag tcctttgaac acatccaaga ctggcaccag gaggtcatgg ccactcaggg
cccggacaag gtcatcttcc tgctggttgg ccacaagagt gacctgcaga gcacccgctg
tgtctcagcc caggaggccg aggagctagc tgcctccctg ggcatggcct tcgtggagac
ctcggttaaa aacaactgca atgtggacct ggcctttgac accctcgctg atgctatcca
gcaggccctg cagcaggggg acatcaagct agaagagggc tgggggggtg tccggctcat
ccacaagacc caaatcccca ggtcccccag caggaagcag cactcaggcc catgccagtg
ttgactctag gagagaaagg gttaaagcag tcccagcctt agcccacctg gtgggatggg
gagtgttaat atctctctgg aggacaaatg acagaagggt tcatataaac agtatcctga
cacagtcatg cttcctggat tttggagtcg aggctttcta cagaaaagaa agttctgatg
gccaggcatg gtggctcacg cctgtaatcc tagcattttt ggaggccaag gacagtggat
cacctgaggt caggggttcg agaccagcct ggccaacatg gtgaaaccct gtctctacta
aaaatacaaa aattagccag gcgtggtggt gcatgcctgt aatcccagtt actccagagg
ctaaggcagg agaattgctt gaacctggga ggcagagatt gcagtgagcc aagactgcgc
cactgcactc cagcctgggc aacagagtga gactctgttt caaaaaaaaa aaagaaaaga
aaagaaaggc ctgagagacc agatgtgcaa cttcctgtcc ttgagcctca gtgtccctat
ctatcgatgg ggctcataaa agatcccacc ttgaagggag gtggtgacca caaatgagac
agtggacagg atgtgctcac ccagagcctg ccgcgctgtg aattgaatga caaaagctct
cattcccact ccctttttct tggctgcgat gtggccactc tggcagcatt cctgggctca
gacactgaga agccagcgtc aggaagctga tgcatgggca aaggcaggtg cggggaattc
cagggggagc ttggcttgga ggcttcttat gtcctcaggc taaaatgatt ctgggcatgg
gattaatatg tgacgtcaaa cccagggttg ctggccaatg cccccccgac caggcccagg
ggctgaaaaa tggatgttgg aggctgggat gaacatgaat gtgtagcaac tatgttgggc
acacagtggc cactgtgatg agccaccaag atcccccttt ctggctgggg aacccatcaa
ccctctcccc agctgctgga gtgccactgg atgatggact tcagcttgcc ccactctctg
ggaaaggccc tcccttcagg gcagcttgta tccaaagttc atctcctggg gggccttaaa
ggactccctc ttgccccagc tctggacaac tctgaaagtc aaaaccaact ttatcagtct
ctgtgggctt cattgaggac actgttgtga catcatagcc aagttatccc cttgcccaat
cctgcttcct tttcttcccc aaacaggtat ccatttcaag aatatcccct aataaacatc
tgcacactca tctcca

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of an Homo sapiens hyperpolarization activated cyclic nucleotide gated potassium channel 3 (HCN3), transcript variant 1, mRNA having NCBI Reference Sequence:

NM_020897.3 (SEQ ID NO: 46):
gattccgagc ctacgacgcc tccgctagag cccgcggggc tgcgccgact cctgctctgg
aggggttgcg ggtacctgat ggccacagag ggctctagga ggccgagcgt gtaagcgggg
tgggcgccat ggaggcagag cagcggccgg cggcgggggc cagcgaaggg gcgacccctg
gactggaggc ggtgcctccc gttgctcccc cgcctgcgac cgcggcctca ggtccgatcc
ccaaatctgg gcctgagcct aagaggaggc accttgggac gctgctccag cctacggtca
acaagttctc ccttcgggtg ttcggcagcc acaaagcagt ggaaatcgag caggagcggg
tgaagtcagc gggggcctgg atcatccacc cctacagcga cttccggttt tactgggacc
tgatcatgct gctgctgatg gtggggaacc tcatcgtcct gcctgtgggc atcaccttct
tcaaggagga gaactccccg ccttggatcg tcttcaacgt attgtctgat actttcttcc
tactggatct ggtgctcaac ttccgaacgg gcatcgtggt ggaggagggt gctgagatcc
tgctggcacc gcgggccatc cgcacgcgct acctgcgcac ctggttcctg gttgacctca
tctcttctat ccctgtggat tacatcttcc tagtggtgga gctggagcca cggttggacg
ctgaggtcta caaaacggca cgggccctac gcatcgttcg cttcaccaag atcctaagcc
tgctgaggct gctccgcctc tcccgcctca tccgctacat acaccagtgg gaggagatct
ttcacatgac ctatgacctg gccagtgctg tggttcgcat cttcaacctc attgggatga
tgctgctgct atgtcactgg gatggctgtc tgcagttcct ggtgcccatg ctgcaggact
tccctcccga ctgctgggtc tccatcaacc acatggtgaa ccactcgtgg ggccgccagt
attcccatgc cctgttcaag gccatgagcc acatgctgtg cattggctat gggcagcagg
cacctgtagg catgcccgac gtctggctca ccatgctcag catgatcgta ggtgccacat
gctacgccat gttcatcggc catgccacgg cactcatcca gtccctggac tcttcccggc
gtcagtacca ggagaagtac aagcaggtgg agcagtacat gtccttccac aagctgccag
cagacacgcg gcagcgcatc cacgagtact atgagcaccg ctaccagggc aagatgttcg
atgaggaaag catcctgggc gagctgagcg agccgcttcg cgaggagatc attaacttca
cctgtcgggg cctggtggcc cacatgccgc tgtttgccca tgccgacccc agcttcgtca
ctgcagttct caccaagctg cgctttgagg tcttccagcc gggggatctc gtggtgcgtg
agggctccgt ggggaggaag atgtacttca tccagcatgg gctgctcagt gtgctggccc
gcggcgcdcg ggacacacgc ctcaccgatg gatcctactt tggggagatc tgcctgctaa
ctaggggccg gcgcacagcc agtgttcggg ctgacaccta ctgccgcctt tactcactca
gcgtggacca tttcaatgct gtgcttgagg agttccccat gatgcgccgg gcctttgaga
ctgtggccat ggatcggctg ctccgcatcg gcaagaagaa ttccatactg cagcggaagc
gctccgagcc aagtccaggc agcagtggtg gcatcatgga gcagcacttg gtgcaacatg
acagagacat ggctcggggt gttcggggtc gggccccgag cacaggagct cagcttagtg
gaaagccagt actgtgggag ccactggtac atgcgcccct tcaggcagct gctgtgacct
ccaatgtggc cattgccctg actcatcagc ggggccctct gcccctctcc cctgactctc
cagccaccct ccttgctcgc tctgcttggc gctcagcagg ctctccagct tccccgctgg
tgcccgtccg agctggccca tgggcatcca cctcccgcct gcccgcccca cctgcccgaa
ccctgcacgc cagcctatcc cgggcagggc gctcccaggt ctccctgctg ggtccccctc
caggaggagg tggacggcgg ctaggacctc ggggccgccc actctcagcc tcccaaccct
ctctgcctca gcgggcaaca ggcgatggct ctcctgggcg taagggatca ggaagtgagc
ggctgcctcc ctcagggctc ctggccaaac ctccaaggac agcccagccc cccaggccac
cagtgcctga gccagccaca ccccggggtc tccagctttc tgccaacatg taaaaccttt
gagtacatcc agccttagtt cttggggtgc agtagtatgt acccaagggc agatgcctct
tggggaaggc catggggacc tgaaacattg ccccatggaa atgtcgaccc tgtgcggaca
ttccgcatac tgccatgaag acggtctctg tgtcctcagc tcaagaatcc tgtagcttgt
cccatcataa tccattcacc cgttcatcat gtgtactgag cagctaccat gttcaaggta
atatgccagg cgctgtatgt ctccactgcc aagtagaagt gactcaaaac cctctgacaa
ggatattccc ttggctatgg tcctgccagg tgcaggccca ggcccatgac cccaccttta
ctaagcacaa gtacttgcca ctgccatcac tgccaagtaa ctagatgtct ctgtttccct
gccaatgatc ctgcaggttc tgcccggtct ggttatcttc ctgttcctgt agcatagcca
ggcactgcca gtcacctgtg cccccattgc tgtcagcaga tgtcttgggt cctgagtgtg
ggtatccact tttacccgct cactgccacc tgtggacact ctgtgtctac cctctgagtg
ggaacatact tctaagttcc ctgcagtctc tgtcctgtgg tagaccatct ttttgtaaac
tgcgagcttc ctcttccctg taccctctgc cccagtcgtg accccctaaa agttaagggg
tagttggcac ctccttatta atatgccagc ctagatcccc cccggtggag gggcaaatgg
ctgaatcctt gtgtgatatt tttttcttcg cttgtttatt tattcattta tttaattgta
tttattcatt tactaacttt atgtgttacc aattaatttt gtttacccat tcctttatcc
atccctcccc tccttttcag gtaaggagac aggaggagta ggaggaggca gggcctctcc
atgccagcct ctgtggtcct tgcccaaacc catcagcgca atacttgaac cttctcccag
gtaggggcag gaggagccac atgagagagg gagaaggacc gcgtttacct ttagagtttt
gttttgtttt ttccttctga gtttgctgtt ggtgcaggaa taagggaaag gcccaaggta
tccaagcctg gggaagggca ggccagccag cacctctgcc ttctcaggga caagagtagt
cctttaccac cctcactctg cctgtcccct ctcctactct acagcattaa agactgtggg
accaggaccc taagtctcct ttccttctgg gtggggagtt ctggggttct tggtgtgtgg
gagaagtttt ataattgctt ccaaacagct gggtttaaat ataaaataga cacactca

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of an Homo sapiens RAS protein activator like 1 (RASAL1), transcript variant 1, mRNA having NCBI Reference Sequence:

NM_001193520.2 (SEQ ID NO: 47):
gtgtttaact ggaaactaga acgagatgga aggggatgtt caaggcccct cccttgactc
tgaacggacc cccagggaac atgcgaccct ctctctggcg acgcctccca cccaccacta
atacttgctc ctggaccggg gggcgcggag gttggagaga ggagctaccc gggtctcgga
caggcggcac tgggaccacg aggcagggag ccaggcttga agcaggtgac atgtagacgt
cccctggtcc agcctcggaa cctgagcgcc cttctgcctg gaaagtttgt ggctaggcgc
catggccaag agcagctccc tgaatgttcg cgtggtggag ggccgcgcgc tgcctgccaa
ggacgtgtct gggagcagcg acccctactg cctagtgaaa gtggacgacg aggtggtggc
caggacagct actgtctgga ggagcctggg ccccttctgg ggggaggagt acacggtgca
cctgcctctg gatttccacc agctggcctt ctacgtgctg gatgaggaca ctgtcgggca
cgacgacatc atcggcaaga tctcgctgag cagggaggcg attacagccg acccccgagg
gattgacagc tggattaact tgagccgagt ggacccagat gcagaagtgc agggtgagat
ctgcctgtca gtgcagatgc tggaggatgg gcagggccgc tgccttcgct gccatgtgct
tcaggccagg gacctggctc ccagagacat ctctggcaca tctgacccat ttgcacgtgt
gttttggggc agccagagct tggagacctc aaccatcaag aagactcgct tcccgcactg
ggatgaagtg ctggagctgc gggagatgcc aggtgccccg tccccactgc gggtggagct
ctgggactgg gacatggtgg gcaagaatga cttcttgggc atggtggagt tctctccaaa
gaccctccag cagaagccac ctaaaggctg gttccgcctc ctgccctttc ccagagccga
ggaggattct ggggggaacc tgggtgccct gcgagtgaag gtacgcctga ttgaggaccg
cgtcctgccc tcccagtgct accagcctct catggagctg ctcatggagt ctgtgcaggg
gccagcagag gaggacactg ctagcccctt ggctttgctg gaagagctga ccttggggga
ctgccgccag gaccttgcca ccaagctggt gaaactcttt cttggccggg gactggctgg
gcgctttctg gactatctca cccggcgtga ggtggctcgg accatggacc ccaacaccct
cttccgttct aactccctgg catccaagtc gatggaacag tttatgaagc tcgtgggcat
gccctacctg cacgaggtcc tgaagcctgt gattagccgt gtctttgagg agaagaagta
catggagctg gatccctgca agatggacct gggccgcacc cggaggatct ccttcaaagg
cgcactctcg gaggagcaga tgcgggagac cagcctgggg ctgctgacgg gctacctggg
gcccatcgtg gacgccatcg tgggctccgt ggggcgctgc ccgcccgcca tgcgcctcgc
cttcaagcag ctgcaccggc gagtggagga gcgcttcccc caggccgagc accagcagga
tgtgaagtac ctggccatca gtggatttct cttcttgcga ttcttcgcac ctgccatcct
taccccaaag ctgtttgacc ttcgggacca acacgcggac ccccagacta gccgctcact
gctgttgctt gccaaggctg tgcagagcat tggaaacctg ggccagcagc tgggccaagg
caaggaactg tggatggccc ccctgcaccc cttcctgctg cagtgtgtct cacgtgtgag
agacttcctg gaccggctgg tggatgtgga tggggatgaa gaagctggtg tcccagccag
ggccctgttc ccgccctcgg ccattgttcg agaaggctat ctgctgaagc gcaaggagga
gcctgccggc ctggccacgc gctttgcctt caagaagcgc tacgtctggc tcagcgggga
gaccctctcc ttctccaaga gtcctgagtg gcagatgtgt cactccatcc ccgtgtctca
catccgcgcc gtggagcgcg tagacgaggg cgccttccaa ctgccccacg tgatgcaggt
ggtgacgcag gacggcacgg gggcgctgca caccacctac ctccagtgca agaatgtgaa
tgagctcaac cagtggctct cggccttgcg caaggccagc gcccccaacc cgaacaagct
ggccgcctgc caccccggtg ccttccgcag cgcgcgctgg acctgctgcc tccaggctga
gcgctcagcc gccggctgca gccgtacaca ctcagctgtc accctggggg actggagtga
cccactggat cctgatgctg aggcccagac agtgtatcgg cagctgctcc tgggggggga
ccagctcagg ctgaaattac tggaggattc taacatggat acaactctgg aggcagacac
aggggcctgt cctgaggtcc tggcccggca aagagcagca actgcccgcc tgctggaggt
gctcgcagac ctggatcgtg cccacgagga gttccagcag caggagcgag ggaaggcggc
cctgggcccc cttggcccct aaggaaatgc cagagctagc ccggaaggag gagcaagagc
cagggggccc tcttcagcgc atcctgcccc gggagtctcc tgtctccttg gacctctttg
attctgtggt ttggaggctc ccagagacgt gcctagtcct gtgtgccttg agtccagaac
tcagggcatg gaagcccttt ggcaggggcc agccttgcac tgagtgaaac ttgccctctg
gcttgattca gactggagtg gataggataa ggaacctgac ttatttgact gagactgggg
tctctacttc accaaactgg cctctatcca taccaaggag gccagcctgg ccctgagctg
ctggatacag ctggacctga attcctgatg cccatgtgat gttgttgccc cagatgggca
ctaaatggcc tcactccttc ctgttttcat gtctgctaat ccctataacc tcactgattc
ttctgtaccc tgcccttggc ctaggactcc aaccacaagc ttccagaatc aggtgccctc
aggaagaacc aaggctgggt gggggtccag tgtgccaaac tcagaccctt ggagcctggg
agaccttggg ccaggctgtt tatctctctc tgggtctcag attaccctgt ataaaaagag
gagggaaagt cta

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of an Homo sapiens UL16 binding protein 1 (ULBP1), transcript variant 1, mRNA having NCBI Reference Sequence:

NM_025218.4 (SEQ ID NO: 48):
gtatccctgc gcgcggcggg ccgggctggg cagctttata aacagccgtg gtgtgagcct
cgaagggaac catcagcgcc tcctgtccac ggagctccag gtctacaatg gcagcggccg
ccagccccgc gttccttctg tgcctcccgc ttctgcacct gctgtctggc tggtcccggg
caggatgggt cgacacacac tgtctttgct atgacttcat catcactcct aagtccagac
ctgaaccaca gtggtgtgaa gttcaaggcc tggtggatga aaggcctttt cttcactatg
actgtgttaa ccacaaggcc aaagcctttg cttctctggg gaagaaagtc aatgtcacaa
aaacctggga agaacaaact gaaacactaa gagacgtggt ggatttcctt aaagggcaac
tgcttgacat tcaagtggag aatttaatac ccattgagcc cctcaccctg caggccagga
tgtcttgtga gcatgaagcc catggacacg gcagaggatc ttggcagttc ctcttcaatg
gacagaagtt cctcctcttt gactcaaaca acagaaagtg gacagcactt catcctggag
ccaagaagat gacagagaag tgggagaaga acagggatgt gaccatgttc ttccagaaga
tttcactggg ggattgtaag atgtggcttg aagaattttt gatgtactgg gaacaaatgc
tggatccaac aaaaccaccc tctctggccc caggcacaac ccaacccaag gccatggcca
ccaccctcag tccctggagc cttctcatca tcttcctctg cttcattcta gctggcagat
gaggagagtt gtttagagtg acaggtggaa agtgatatca agaagcctct gttagcctgg
tctgggtcct gctctccctt cagggaggcc gcctgtctac tcaccactgt gcctttctgg
aaagcaggag ttcaagcctt agcaagccca gaggccccca gcagacgatg aggacattgt
cggctcaaca tctcaggcca ctcattacct tcgctcatga tcccagcagc catttttctt
aacaccttct gccactttct gtcggtgcta atggatggaa ctcctgcaca agttttaact
gaacaagaaa tcccagcaaa aggcattttt ttttctactt ctttgattgt agaaaagcag
acacttctct gaaacatgac cttattcttc caaacagtat cgctagtaaa atagcatgct
ggacttcaga cctcagggat ccttttgatg cactgaccag gaattgtgat aatccttttc
atttttatgg ctttttacag tttctcattc tgtcaaccat attgaagtga agtggcatag
tcttcactca ctgtaatctc cagctcctgg gctcaagtga tcctctagac tcagcctcta
gaatagctgg gactacaggc acatgccacc aggactggca aactgtttta tttttaggta
gaggtagggt cttcctatgt tgcccaggat ggtcttcaaa tcttggtctc gagcaatgct
accaccttgg cctcccaatg ctctgggatt acagacatga accacagtgc ctgttgtaga
aatttttaat tatttaatat gaaaatatta cattcatgat tattttattt agtaaataaa
ataatagaga gcccagaaat caacctgcac acctaccgcc atctaatctt caatagaaat
gggcaatgtg ggaaagactc cctattcgaa aattagtgct ggaatatctg gccaaccata
tgcagaagaa tgaaactgaa cccctacttc tccccatata tgtaaaataa ttcaatatgg
atgaaagatt taaatataag tactaaaact gtaaaaatcc tggaatataa cctaagaaat
accaatgtgg acatagggcc tggcaaagat ttcatgaaga agacactaaa aacaattqca
acaaaaacag aaattgacaa atggggcctc attaaactaa agagcttctg cacagaaaaa
gaaactagca acacagtaaa cagacagcct gtagaatggg gaaaactatt tgcaaactct
gcatctgatg aaggtccaat atccagaatc tacaaggaac ttaaacaatt caacaagcaa
gaagaaaaaa cccaattaaa attgggcaaa ggcatgaaca gacacttttc aaaagaagac
ctacaattgg ccaacaaaca tgaaaaaatg ctcagtatca ctaatcatca gataagtgca
aatcaaaact gcaatgagtt accatctctt accagtcaca aagtcagaga tggtggtgag
gctgcagagc aaaagaaaca gacactgttg gtgggaaagc aaacttgttc agccactatg
gaaagcagtt tggagatttc tccaagaact taaaatagaa ctaccattca atcccgcaat
cccactactc gggatatacc cacaggaaaa gaattcattt tatcaaaaag acacctgcac
caatatgttc attacagtgc tattctcacc agcaaggaca gagaatcaat ctaagtgccc
aacaacagta aattcaatga aaaaaaatgt ggtacataga tacgatggaa aactatgcag
ccatgaaaca caagaaaatc atgtcctttt cagcaacatg gatgcaacta gaggctatta
tcctaagcaa cctaatgcaa gaacagaaaa ccacatactg catcttccca ttggaaagtg
gcagctaaac attaaattcg catgaaccac agatgctgga gatcaccaga ccggggagag
aagaggggca cctgggctga aaaacacacc tgttgggtat catgcttact gtctgggcga
tgggatcatt gggacaccaa gcctcagctt ctcaaattct acccatgtaa caaacctgta
tatgtacctt gtattatata ggttgaaatt aaagatgaat aaataaaata aaatgacaca
aggccaaaaa caaatgggtt taactgacca gagcgagaga actctgcact atgaacccaa
acccagctca aaaagataaa atctagtcat ttaagataat cataagttgt atgatgataa
ttgtataaaa atttgtatga tgataattgt ataataatta tacatgaaag tcccaaaacc
ctacaattaa acactgtata atggaattac a

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of an Homo sapiens macrophage immunometabolism regulator (MACIR), transcript variant 1, mRNA having NCBI Reference Sequence:

NM_001316968.2 (SEQ ID NO: 49):
cccttggccg ctgttcccgc accgcggggc agcgggcctg gaggcccctt tgagaagtag
ctttccccgg ccggcggcac ctttggctgc gtgcccggcc gcgctcaggg tgcgactgcc
cgggtcagat agcacctcag ggcgagcccc ggcggtctga tctcgccgac cctcctcgtc
ctggttgcaa cccgcgtgcg aggccgcccg cgccctccga gtgtctgccg gtgcagtggg
ggtggactgg ccggctgtgg ccgtgtgtgc gcgtgtggat tggggcccgg tccgagccag
aagcttaagc ggcagatgtc gggcattgcc accctcgccc cacctgtcgc gggtggactt
tggggcagta cctggagtag aacagaaaaa ttattatgtc tgtgttccct tgggactcat
tggaaattgt acagtgacat cttctgggat ttagtctgga ttgtgcagac tggtgcttaa
aatggaagtc gatattaatg gagagtctag aagtaccctg accaccttgc ccttccctgg
ggctgaggcc aactccccgg gaaaggcgga ggcagagaag ccccgctgct ccagcacacc
ctgctccccg atgcggagga ccgtgtcagg ctaccagatc ctacacatgg actctaacta
tttggttggc ttcacgactg gcgaggaact cctgaagtta gctcagaagt gcacaggagg
tgaagagagc aaagcagaag ccatgccatc cttacgctcc aaacagctag atgcaggact
tgcccgttcc tctcgtttgt ataaaaccag aagtaggtac taccagccat acgagattcc
agctgtcaat ggcaggaggc gaaggcggat gccaagctca ggagacaagt gcactaaatc
tttaccttat gaaccttaca aggccctcca tgggcctctg cctctttgtc ttcttaaagg
taagagggct cactccaaat ctctggacta cctcaatcta gataaaatga tcaaggagcc
agctgataca gaagtgctac agtaccagct tcaacaccta accctccgag gggaccgtgt
gtttgctagg aataatacat gaatgacttg gagagagctt aaaccaattt aggtcagcct
acgcttggct agaaaaaacc cactgctgta ctctgtacat gactcttcac actatagatg
gttatatcag ctaagtgttc ctggaacata aaaattgttt gggtcaaatt tgaatacagg
aatgaaatca caggtacttg ggggggggat atcattctag agcacgcaac tgcaaagaaa
acagaatgtt gactgttagt ttgtatagct ttttagctag gaaaaaaagg ctttggtaca
gtaatttcat ctttatgatt ctgacactca aattgggaat gttacctgct tggttgttgc
tgacttgata tgattcatta gaaatttata tcttcagtac tcaagtactt cttgaatctc
tgtattttac tataaaatgt atgtaatgat ttgttttatg aaatttagaa cttgaacatt
gctgaattgg accacttttt atttttaaat attgagttta aatattttat aactggtttt
gcactgaaaa aattaacatt tcagattgac aagagagtaa tctttcttca cttgcctcaa
taatgttatt gagcaatgaa ttttttattt ccgcatggaa agttattgat ctctatggct
gtaaaatatt tctttatagc gttattaaag tgtgtcttaa taaaattaaa tttgggatac
aaagtattta ttttacaatg ggtgggcggg ggaaactttt ccagaaagtt tccaatatga
cgttttcata agttgaaaaa actctcctta gtgcttattt tctaacttaa aattcacctg
gaactttaaa tgggaaagga ttcttttaat tgtggattat aggcataata ctgtttgcat
ctgaattttc tgtaagtgaa taatagttta atagaggaac tcatgatttg tactattgaa
tgattaaact aagtatgaag tgataccatt cagcatggca tcaggtcatt gcagttttag
ttctgtgtaa cacaagcact cactgaaatt ccagtttcta ggattagtgt aggagcctaa
cgtgcttcta ctgttttaat gggttaatcc tggattactt aacaatttat gtcaattgca
ctggtttaat ttgttgctaa agaaataatg ccctgggttt agtaacaaat acagctcaac
tattcttgaa tatattttga aaaaaaaatg tatgtaactt accttttgta aacgttccat
ttcttttttc cctcattttt gactcttaaa ggtgcaattt attactgaat tgggatttct
ggcagcacag aactgctttt tattttgggg tctgtgagtt tcttaggtat tagcaatctt
gcttataaaa taagaacacc ttttaattaa tgagtgggtc attcctggtg caattgtgat
ttttctttag ccagaatgaa tggcaaactc tatttagagc aaagtaagta ttagaaaacc
ctaggaactc ttaatcaacg tttattacac tttcattaag gcaaactacg tgaaagagcc
ttggggaagt tggcccatat cttactaagt tgatcagatt tctcgttggg ctggaaatgt
ttcgctgttg tatattttaa agtaaattgc acctttgtaa catattgtat tgacgaatga
tcactaagat tagctatatc tatacagtca ttagtttgac aagaaataga atcctgtcag
atgccaaaga gtgggatttt tatgtttaat gattaaacac cattatttat tgacaattta
ccctgtggaa ctgtattatt tctaactatg aaataaaggg gtgatgtaaa cacacattgt
tgtgtggtgc tttaaactag gtccactatc aacaggctac ttactgttca agaattccac
tgaagcactt attttaaggc cctatttttc ttaaacaaaa cagtgacaac aacaatcaaa
ccatttactt ttgatgctca ttggcatttt atgataaaag atgtattcat ggcaatgata
tgtattcacc ctattaggaa acacaactgg ttacctatga gacctgttct gtccgtgtgc
ctacgttcct taataatagc taaataaaaa tttgtagctt tt

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of an Homo sapiens poly(ADP-ribose) polymerase family member 15 (PARP15), transcript variant 1, mRNA having NCBI Reference Sequence:

NM_001113523.3 (SEQ ID NO: 50):
agtcccagcg agctgaggat ggctgcgcca ggcccccttc ctgccgctgc tctgagtcca
ggggctccga cccccagaga acttatgcac ggagttgcag gtgttacttc cagagccgga
cgagatcggg aggcggggag cgtgctgccg gccgggaacc gtggggcgcg gaaggcctcc
cggcgctctt cctcccggag tatgtccaga gacaacaagt tcagcaagaa agattgtctt
tcaatcagga atgttgtagc ttcaatccaa accaaagaag gtctgaatct caagttgata
agtggagatg ttctgtacat ctgggccgat gtcattgtca acagcgttcc catgaatctt
cagcttggag gaggaccact atctcgggca tttttgcaga aagctggtcc catgctccag
aaagagttag atgacagaag gcgggaaaca gaggaaaaag taggtaacat attcatgaca
agcggctgca atctggactg caaagctgtg ctccatgctg tggctccata ctggaataat
ggagcagaga cttcttggca gatcatggca aatataatca agaaatgttt gacaactgta
gaagtgctat ctttctcatc aatcacattt cccatgattg gaacaggaag tttgcagttt
cccaaagctg tttttgctaa actaatcctt tcagaagtgt tcgaatacag tagcagcaca
aggccgataa ctagcccttt acaagaagtc cactttctgg tatatacaaa tgacgatgaa
ggctgtcagg catttttaga tgaattcact aactggtcaa gaataaatcc caacaaggcc
aggattccca tggcaggaga tacccaaggt gtggtcggga ctgtctctaa gccttgtttc
acagcatatg aaatgaaaat cggtgcaatt acttttcagg ttgctactgg agatatagcc
actgaacagg tagatgttat tgtaaactca acagcaagga catttaatcg gaaatcaggt
gtgtcaagag ctattttaga aggtgctgga caagctgtgg aaagtgaatg tgctgtacta
gctgcacagc ctcacagaga ttttataatt acaccaggtg gatgcttaaa gtgcaaaata
ataattcatg ttcctggggg aaaagatgtc aggaaaacgg tcaccagtgt tctagaagag
tgtgaacaga ggaagtacac atcggtttcc cttccagcca ttggaacagg aaatgccgga
aaaaacccta tcacagttgc tgataacata atcgatgcta ttgtagactt ctcatcacaa
cattccaccc catcattaaa aacagttaaa gttgtcattt ttcaacctga gctgctaaat
atattctacg acagcatgaa aaaaagagac ctctctgcat cactgaactt tcagtccaca
ttctccatga ctacatgtaa tcttcctgaa cactggactg acatgaatca tcagctgttt
tgcatggtcc agctagagcc aggacaatca gaatataata ccataaagga caagttcacc
cgaacttgtt cttcctacgc aatagagaag attgagagga tacagaatgc atttctctgg
cagagctacc aggtaaagaa aaggcaaatg gatatcaaga atgaccataa gaataatgag
agactcctct tccatgggac agatgcagac tcagtgccat atgtcaatca gcacggcttt
aatagaagtt gtgctgggaa aaatgctgta tcctatggaa aaggaaccta ttttgctgtg
gatgccagtt attctgccaa ggacacctac tccaagccag acagcaatgg gagaaagcac
atgtacgttg tgcgagtact tactggagtc ttcacaaagg gacgtgcagg attagtcacc
cctccaccca agaatcctca caatcccaca gatctctttg actcagtgac aaacaataca
cgatctccaa agctatttgt ggtattcttt gataatcagg cttacccaga atatctcata
actttcacgg cttaaaaata tttttatcat caaagagatg atttaagtca tctgtaagaa
caacatgcaa tctttgtctt tgcttctggc ctgtgtaagc agatgaaagt ttccctttta
ggtgccaaaa tgctgaaaat taccttttta aagtgctcta ttgctgcgat ttgtagcata
cctttttttc tcagcaaatt gatgggtgga agctgagaaa tgtatggtaa atgtcacaga
gctacaacca ttcacagaca ccaaatctct aggagaataa aaagcacatt attctttttc
tatcagaaaa aaacaagatg catcacctta aaaccaagat gacattgttc ttcttggaac
atgttaagac atcgaatggt ggcgggttaa actgtactgc ttaagtggag cggctaccgt
tatgcatcta tcacagttgg ggattttgcc ttattaagga aaacttgtca atagttcagc
tgaaatgact gaatcacaga atattaactc tgttatggaa caaatcataa cagattttac
ctgtttacat ttcaggtaaa aatgtatcgc attgttatct aatattaaaa aattaccccc
aattttagtg acttaatccc acacagtctt tatgggtcag gaattcaggc atggcttacc
tggatcattc tgctagggtc tctctgaagt tacagacaag atgtcagggg atgtggtcgt
ttgaaggctt gtctgggctg gaggtctatt tccaaggtga atcactcaca tacctggcac
gtttctgtca ggtattggca gtcctcagtt cctctcctct caggcctctc cacaggctgc
ttgagtgtcc tcatgacaca acagttggct tactccagag tgagcaactc aagagagagc
aaggcagaag ctaccaaatc tttatgtttg aagtcatgca ccatcttttc cacgagtatc
ctgttgatta ttttgatcag cttgttcagt ctgggaggga attgcacaag ggcatgaata
ctccactggc aaggatcatt gggggccatc ttggaagctg tgtgaatgag caaatgaatg
cacagataga atattagcag tgacaatgat gctagaggtc acctacccca ctgtcctctt
gtccttctcc cccaaccctc ccctgctccc aggcaagaag ccctctagcc tctgcttgat
cactttcagc actcaacatc ttcagggaac ctattccgcc gtgggacagt gttaattagt
ggaaaactct ttttcaaaag ttgaaatcag ttcctctgtg tctattacct gctgatcact
gtccagactt ctggaggaca cagagcaagt tttattcctc ttactgatgg tagcctttca
gatccatccc ttccctccag tatattagag ttacgtaaat tcttaaaatg cttagcagct
catttatcct gaagcatcac ttttgaagag ttacagacat ttaagaagta tttacattat
cataaataaa ttacatgcgc atttaaagag aacagaaaag tataaagaaa aataactcac
caaactcact cataattcca gcactaaggg aaaaccactg ccaatttctc atgtctccct
tccagtttct tctctgtcca aattcaacaa agtcaaaatc acattttgtt ctgtcacaaa
tcatataata ttatttctac catttttctt tttttaatct ttttttatta tactttaagt
tttagggtac atgtgcacaa tgtgcagttt agttacatat gtaaacatgt gccatgttgg
tgtgctgcac ccagtaactc atcatttagc attaggtata tctccaaatg ctatccctct
cccctctccc caccccacaa caggccccgg tgtgtgatgt tccccttcct gtgtccatgt
gttctcattg ttcaattccc acctgtgagt gagaacatgt ggtgtttggt tttttgtcct
tgcgatagtt tgctgagaat gatggtttcc agcttcatcc atgtccctac aaaggacatg
aactcatcat tttttatggc tgcatagtat tccatggtgt atatgtgcca cattttctta
atccagtcta tcattgttgg acatttgggt tggttccaag tctttgctat tgtgaatagt
gccgcaataa acatacgtgt gcatgtgtct ttacagcagc atgatttata atcctttggg
tatataccca gtaatgggat tgctgggtca aatggtattt ctagttctag atccccgagg
aatcgccaca ctgacttcca caatggttga actagtttac agtcccacca acagtgtaaa
agtgttccta tttctccaca tcctctccag cacctgttgc ttcctaactt tttaatgatt
gccattctaa cgggtgtggg atggtatctc attgttgttt tgatttgcat ttctctgatg
gccagtgatg atgagcattt tttcatgtgt tttttggctg cataaatgtc ttcttttgag
aagtgtccgt tcatatcctt tgcccacttt ttgatggggt tctttgtttt tttcttgtat
atttgtttga gttcattgta gattctggat attagccctt tgtcagatga gtagattgca
aaaattttct cccattctgt aggttgcctg ttcactctga tggtagtttc ttttgttgta
cagaagctct ttagtttaat tagatcccat atttctacca tttttcatta aacattacaa
gttgttctct tgtgttctta ttttttctgt aaacataatt ttaatggcag agatattctg
ctttatatct ttccctattt tatgtatata gaattataaa gtttttaaaa atgtaatcat
cattatttat agtttaataa tcagtttcat cttgcagtgt atcattttcc tcgtcattaa
atattttacg tatcattttt aa

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of an Homo sapiens neuroligin 4 X-linked (NLGN4X), transcript variant 1, mRNA having NCBI Reference Sequence.

(SEQ ID NO: 51):
NM_020742.4
atttaagcga ttttttttcc ctccttcatc tccgggcctc
ggataagatg acggcttggg tgatgcacga aataacgcac
gtgattgatt agacctggct tggcttggct agggaacgat
ccaggcgcgc tggagacccc gcgtgaagat gaaatgacgg
ctgccttgga gttttcataa gaaattgtcc ctggaggtgt
tggatgatca cagcttcctt ggagcattgc agttgctgga
atccagtttc aggattaagg gagggctgcc tccttgcaat
gggctgccaa gaaaacggct gtgcttgttc ttaacctcag
gctctgtctg tgatcagtct gagagtctct cccaggtcta
ctgctccctg gaaagcccta tctctctgca ggctcgcctc
tgggctttgt ctccttggag ccacatcact gggacagctg
tcgatgtgga tgcagatttg aaccatgtca cggccccagg
gactgctatg gcttcctttg ttgttcaccc cggtctgcgt
catgttaaac tccaatgtcc tcctgtggtt aactgctctt
gccatcaagt tcaccctcat tgacagccaa gcacagtatc
cagttgtcaa cacaaattat ggcaaaatcc ggggcctaag
aacaccctta cccaatgaga tcttgggtcc agtggagcag
tacttagggg tcccctatgc ctcacccccc actggagaga
ggcggtttca gcccccagaa cccccgtcct cctggactgg
catccgaaat actactcagt ttgctgctgt gtgcccccag
cacctggatg agagatcctt actgcatgac atgctgccca
tctggtttac cgccaatttg gatactttga tgacctatgt
tcaagatcaa aatgaagact gcctttactt aaacatctac
gtgcccacgg aagatgatat tcatgatcag aacagtaaga
agcccgtcat ggtctatatc catgggggat cttacatgga
gggcaccggc aacatgattg acggcagcat tttggcaagc
tacggaaacg tcatcgtgat caccattaac taccgtctgg
gaatactagg gtttttaagt accggtgacc aggcagcaaa
aggcaactat gggctcctgg atcagattca agcactgcgg
tggattgagg agaatgtggg agcctttggc ggggacccca
agagagtgac catctttggc tcgggggctg gggcctcctg
tgtcagcctg ttgaccctgt cccactactc agaaggtctc
ttccagaagg ccatcattca gagcggcacc gccctgtcca
gctgggcagt gaactaccag ccggccaagt acactcggat
attggcagac aaggtcggct gcaacatgct ggacaccacg
gacatggtag aatgcctgcg gaacaagaac tacaaggagc
tcatccagca gaccatcacc ccggccacct accacatagc
cttcgggccg gtgatcgacg gcgacgtcat cccagacgac
ccccagatcc tgatggagca aggcgagttc ctcaactacg
acatcatgct gggcgtcaac caaggggaag gcctgaagtt
cgtggacggc atcgtggata acgaggacgg tgtgacgccc
aacgactttg acttctccgt gtccaacttc gtggacaacc
tttacggcta ccctgaaggg aaagacactt tgcgggagac
tatcaagttc atgtacacag actgggccga taaggaaaac
ccggagacgc ggcggaaaac cctggtggct ctctttactg
accaccagtg ggtggccccc gccgtggcca ccgccgacct
gcacgcgcag tacggctccc ccacctactt ctatgccttc
tatcatcact gccaaagcga aatgaagccc agctgggcag
attcggccca tggtgatgag gtcccctatg tcttcggcat
ccccatgatc ggtcccaccg agctcttcag ttgtaacttt
tccaagaacg acgtcatgct cagcgccgtg gtcatgacct
actggacgaa cttcgccaaa actggtgatc caaatcaacc
agttcctcag gataccaagt tcattcacac aaaacccaac
cgctttgaag aagtggcctg gtccaagtat aatcccaaag
accagctcta tctgcatatt ggcttgaaac ccagagtgag
agatcactac cgggcaacga aagtggcttt ctggttggaa
ctcgttcctc atttgcacaa cttgaacgag atattccagt
atgtttcaac aaccacaaag gttcctccac cagacatgac
atcatttccc tatggcaccc ggcgatctcc cgccaagata
tggccaacca ccaaacgccc agcaatcact cctgccaaca
atcccaaaca ctctaaggac cctcacaaaa cagggcctga
ggacacaact gtcctcattg aaaccaaacg agattattcc
accgaattaa gtgtcaccat tgccgtcggg gcgtcgctcc
tcttcctcaa catcttagct tttgcggcgc tgtactacaa
aaaggacaag aggcgccatg agactcacag gcgccccagt
ccccagagaa acaccacaaa tgatatcgct cacatccaga
acgaagagat catgtctctg cagatgaagc agctggaaca
cgatcacgag tgtgagtcgc tgcaggcaca cgacacactg
aggctcacct gcccgccaga ctacaccctc acgctgcgcc
ggtcgccaga tgacatccca cttatgacgc casacaccat
caccatgatt ccaaacacac tgacggggat gcagcctttg
cacactttta acaccttcag tggaggacaa aacagtacaa
atttacccca cggacattcc accactagag tatagctttg
ccctatttcc cttcctatcc ctctgcccta cccgctcagc
aacatagaag agggaaggaa agagagaagg aaagagagag
agaaagaaag tctccagacc aggaatgttt ttgtcccact
gacttaagac aaaaatgcaa aaaggcagtc atcccatccc
ggcagaccct tatcgttggt gttttccagt attacaagat
caacttctga ccctgtgaaa tgtgagaagt acacatttct
gttaaaataa ctgctttaag atctctacca ctccaatcga
tgtttagtgt gataggacat caccatttca aggccccggg
tgtttccaac gtcatggaag cagctgacac ttctgaaact
cagccaagga cacttgatat tttttaatta caatggaagt
ttaaacattt ctttctgtgc cacacaatgg atggctctcc
ttaagtgaag aaagagtcaa tgagattttg cccagcacat
ggagctgtaa tccagagaga aggaaacgta gaaatttatt
attaaaagaa tggactgtgc agcgaaatct gtacggttct
gtgcaaagag gtgttttgcc agcctgaact atatttaaga
gactttgtaa aaaagaaaaa tgtatatagc tgtgagttta
aacaaaaacc acaaacagac aaacaagaaa aaaagctttt
attggtgttt tcactttgaa agagctttta gcaaggttgt
gcttttcatt gtgctctgta cgtatataaa tatatatata
tatacacaca cacacacaca ttagtcatat cacctctgtt
tcctccccaa caaaagaggc ttttcttctt aattacttgt
ggtaaacaaa gacatgggat tttcttacat gagattctca
tttgtaggag gatgtgatgt cccacagaag acccagacgg
tctgtgtggc ctatttcccc cgtcaggttg cacaggtgca
tgcaagagca ttcttaggag accactgttt tgaaaaactt
ttgacttgta cgtgttagcc ttcatgaaat tgcagtacag
agatgggtcc ccaaagtgga gtgtatttac agcttgttaa
attagagaca tgcacacaca aagaatcagt agggagaaac
aaaaatacaa gtcccgttct gtagctctgg ccctttgaat
atgtttagga agagttgctt cccatttcag ggccctgcca
aaaaaagaag aaagcttgcc tttggtgggg ctatgcccct
tggagtaaat acggctctgt gttccctagc agctgcggga
gggtttggcc gatgaagtac ctgctcagct tagctaatca
gattgaagga agacatgtgt ctttcctttt tgtttaagca
ctcggtccct tatttatcag taagcaggtt tttaaaaatc
ttttatatca tttatgggat caaacatatg attgtctgaa
aacatcactt tttgtggatt tgtgtatccg gtcaccaaac
ggtgaatatt atagaagaat gggggaagaa aggatagaat
attaaaactg ctttgcatgg gttttctggg aaattaggat
aacttcactg agaagacatt gaatggaaat tattcaccca
ttttaaattg gtgacctagg gatcagagat ttgtctttcc
aacagcttgt cattttttca tttctcttct catttttcag
gaaagttttg agtgttataa ggtggaagga aacatagtag
caatggatac ttttttgaaa aattattgca ttaccaagaa
acagtagcca aagatatttg aagatcatgt tcctcggctc
cattgtgggt tattctagaa atccagtctt aaatctctcc
gctaaagtgg acattcccca taaaaattgt ccagctgcct
ggctcttttg caataacaac ctttgattac tgaatcccta
cactcaaact atagtgatat atcagtgttt gagagtgacc
tctagaaaaa agaaaagtgt ttttagaaat gcgtacaagt
cacccccaaa tcctattgct tatcttgggt taaatttgag
agtgattctc tgtatataaa tatgtgaaat attattatct
caacttagca cacgtgaagc aacatttctt tcctacagag
aggtgtcatg gtaagatttc attccgaatt cattgtttca
tagagctatg atcaggccat ttctgcaagc aatgtatgac
cccacctgag caaccacaaa taggctctct gtgaaactac
aaaggaagtt atgtgtggca tccatgttgg tttcgtctgt
ctgtaatgtg aattccagta tttgtttagt atttccagtt
gtctcctgct agcaatatgt acagtaacgc gtcaggcttg
tgacatttga ataaggaaaa acagagttcc tgttaagtga
ataactttag cttttacagg ggattatgat caaaagtgat
tttagtacat cttaaatgat atcttatttc tacatggaaa
gaagttatag aatcttcata gagttctatg agaaaaaata
tacttgctat ctataaaaaa gagaaaaaag aaaaaaaatg
agaaaaaagt aagaaaaaaa aaaatcctgt cctaggcttt
tactcttgat cttcaaaggc acgcagggtt taatggttcc
ttgggttatt attttgcagt tttgtttttt attttgcctt
aagtaatgat agaagatata tatggccgga cacatatgta
taaacttttc agcagcattt ttaataataa aatatcacag
tattttctaa

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of an Homo sapiens CD59 molecule (CD59 blood group) (CD59), transcript variant 1, mRNA having NCBI Reference Sequence:

NM_203330.2 (SEQ ID NO: 52):
ggggccgggg ggcggagcct tgcgggctgg agcgaaagaa
tgcgggggct gagcgcagaa gcggctcgag gctggaagag
gatcttgggc gccgccagtc tctctctgtt gcccaagctg
gagtgcagtg gcacagtctt ggctcactgc aacctccacc
tcctgggtgc aagcgattct cgtgtctcag cctctcaagt
agctgggatt acagtcttta gcaccagttg gtgtaggagt
tgagacctac ttcacagtag ttctgtggac aatcacaatg
ggaatccaag gagggtctgt cctgttcggg ctgctgctcg
tcctggctgt cttctgccat tcaggtcata gcctgcagtg
ctacaactgt cctaacccaa ctgctgactg caaaacagcc
gtcaattgtt catctgattt tgatgcgtgt ctcattacca
aagctgggtt acaagtgtat aacaagtgtt ggaagtttga
gcattgcaat ttcaacgacg tcacaacccg cttgagggaa
aatgagctaa cgtactactg ctgcaagaag gacctgtgta
actttaacga acagcttgaa aatggtggga catccttatc
agagaaaaca gttcttctgc tggtgactcc atttctggca
gcagcctgga gccttcatcc ctaagtcaac accaggagag
cttctcccaa actccccgtt cctgcgtagt ccgctttctc
ttgctgccac attctaaagg cttgatattt tccaaatgga
tcctgttggg aaagaataaa attagcttga gcaacctggc
taagatagag gggctctggg agactttgaa gaccagtcct
gtttgcaggg aagccccact tgaaggaaga agtctaagag
tgaagtaggt gtgacttgaa ctagattgca tgcttcctcc
tttgctcttg ggaagaccag ctttgcagtg acagcttgag
tgggttctct gcagccctca gattattttt cctctggctc
cttggatgta gtcagttagc atcattagta catctttgga
gggtggggca ggagtatatg agcatcctct ctcacatgga
acgctttcat aaacttcagg gatcccgtgt tcccatcgag
gcatgccaaa tgttccatat gtgggtgtca gtcagggaca
acaagatcct taatgcagag ctagaggact tctggcaggg
aagtggggaa gtgttccaga tagcagggca tgaaaactta
gagaggtaca agtggctgaa aatcgagttt ttcctctgtc
tttaaatttt atatgggctt tgttatcttc cactggaaaa
gtgtaatagc atacatcaat ggtgtgttaa agctatttcc
ttgccttttt tttattggaa tggtaggata tcttggcttt
gccacacaca gttacagagt gaacactcta ctacatgtga
ctggcagtat taagtgtgct tattttaaat gttactggta
gaaaggcagt tcaggtatgt gtgtatatag tatgaatgca
gtggggacac cctttgtggt tacagtttga gacttccaaa
ggtcatcctt aataacaaca gatctgcagg ggtatgtttt
accatctgca tccagcctcc tgctaactcc tagctgactc
agcatagatt gtataaaata cctttgtaac ggctcttagc
acactcacag atgtttgagg ctttcagaag ctcttctaaa
aaatgataca cacctttcac aagggcaaac tttttccttt
tccctgtgta ttctagtgaa tgaatctcaa gattcagtag
acctaatgac atttgtattt tatgatcttg gctgtattta
atggcatagg ctgacttttg cagatggagg aatttcttga
ttaatgttga aaaaaaaccc ttgattatac tctgttggac
aaaccgagtg caatgaatga tgcttttctg aaaatgaaat
ataacaagtg ggtgaatgtg gttatggccg aaaaggatat
gcagtatgct taatggtagc aactgaaaga agacatcctg
agcagtgcca gctttcttct gttgatgccg ttccctgaac
ataggaaaat agaaacttgc ttatcaaaac ttagcattac
cttggtgctc tgtgttctct gttagctcag tgtctttcct
tacatcaata ggtttttttt tttttttttg gcctgaggaa
gtactgacca tgcccacagc caccggctga gcaaagaagc
tcatttcatg tgagttctaa ggaatgagaa acaattttga
tgaatttaag cagaaaatga atttctggga acttttttgg
gggcgggggg gtggggaatt cagccacact ccagaaagcc
aggagtcgac agttttggaa gcctctctca ggattgagat
tctaggatga gattggctta ctgctatctt gtgtcatgta
cccacttttt ggccagacta cactgggaag aaggtagtcc
tctaaagcaa aatctgagtg ccactaaatg gggagatggg
gctgttaagc tgtccaaatc aacaagggtc atataaatgg
ccttaaactt tggggttgct ttctgcaaaa agttgctgtg
actcatgcca tagacaaggt tgagtgcctg gacccaaagg
caatactgta atgtaaagac atttatagta ctaggcaaac
agcaccccag gtactccagg ccctcctggc tggagagggc
tgtggcaata gaaaattagt gccaactgca gtgagtcagc
ctaggttaaa tagagagtgt aagagtgctg gacaggaacc
tccaccctca tgtcacattt cttcaatgtg acccttctgg
cccctctcct cctgacagcg gaacaatgac tgccccgata
ggtgaggctg gaggaagaat cagtcctgtc cttggcaagc
tcttcactat gacagtaaag gctctctgcc tgctgccaag
gcctgtgact ttctaacctg gcctcacgct gggtaagctt
aaggtagagg tgcaggatta gcaagcccac ctggctacca
ggccgacagc tacatcctcc aactgaccct gatcaacgaa
gagggattca tgtgtctgtc tcagttggtt ccaaatgaaa
ccagggagca ggggagttag gaatcgaaca ccagtcatgc
ctactggctc tctgctcgag agccaatacc ctgtgccctc
cactcatctg gatttacagg aactgtcata gtgttcagta
ttgggtggtg ataagcccat tggattgtcc ccttgggggg
atgagctagg ggtgcaagga acacctgatg agtagataag
tggagctcat ggtatttcct gaaagatgct aatctatttg
ccaaacttgg tcttgaatgt actgggggct tcaaggtatg
ggtatatttt tcttgtgtcc ttgcagttag cccccatgtc
ttatgtgtgt cctgaaaaaa taagagcctg cccaagactt
tgggcctctt gacagaatta accactttta tacatctgag
ttctcttggt aagttcttta gcagtgttca aagtctacta
gctcgcatta gtttctgttg ctgccaacag atctgaacta
atgctaacag atccccctga gggattcttg atgggctgag
cagctggctg gagctagtac tgactgacat tcattgtgat
gagggcagct ttctggtaca ggattctaag ctctatgttt
tatatacatt ttcatctgta cttgcacctc actttacaca
agaggaaact atgcaaagtt agctggatcg ctcaaggtca
cttaggtaag ttggcaagtc catgcttccc actcagctcc
tcaggtcagc aagtctactt ctctgcctat tttgtatact
ctctttaata tgtgcctagc tttggaaagt ctagaatggg
tccctggtgc ctttttactt tgaagaaatc agtttctgcc
tctttttgga aaagaaaaca aagtgcaatt gttttttact
ggaaagttac ccaatagcat gaggtgaaca ggacgtagtt
aggccttcct gtaaacagaa aatcatatca aaacactatc
ttcccatctg tttctcaatg cctgctactt cttgtagata
tttcatttca ggagagcagc agttaaaccc gtggattttg
tagttaggaa cctgggttca aaccctcttc cactaattgg
ctatgtctct ggacaagttt tttttttttt ttttttttaa
accctttctg aactttcact ttctatgtct acctcaaaga
attgttgtga ggcttgagat aatgcatttg taaagggtct
gccagatagg aagatgctag ttatggattt acaaggttgt
taaggctgta agagtctaaa acctacagtg aatcacaatg
catttacccc cactgacttg gacataagtg aaaactagcc
agaagtctct ttttcaaatt acttacaggt tattcaatat
aaaatttttg taatggataa tcttatttat ctaaactaaa
gcttcctgtt tatacacact cctgttattc tgggataaga
taaatgacca cagtacctta atttctaggt gggtgcctgt
gatggttcat tgtaggtaag gacattttct ctttttcagc
agctgtgtag gtccagagcc tctgggagag gaggggggta
gcatgcaccc agcaggggac tgaactggga aactcaaggt
tctttttact gtggggtagt gagctgcctt tctgtgatcg
gtttccctag ggatgttgct gttcccctcc ttgctattcg
cagctacata caacgtggcc aaccccagta ggctgatcct
atatatgatc agtgctggtg ctgactctca atagccccac
ccaagctggc tataggttta cagatacatt aattaggcaa
cctaaaatat tgatgctggt gttggtgtga cataatgcta
tggccagaac tgaaacttag agttataatt catgtattag
gcttctccag agggacagaa ttagtaggat atatgtatat
atgaaaggga ggttattagg gagaactggc tcccacagtt
agaaggcgaa gtcgcacaat aggccgtctg caagctgggt
tagagagaag ccagtagtgg ctcagcctga gttcaaaaac
ctcaaaactg gggaagctga cagtgcagcc agccttcagt
ctgtggccaa aggcccaaga gcccctggca accaacccac
tggtgcaagt cctagattcc aaaggctgaa gaacctggag
tctgatgtcc aagagcagga agagtggaag aaagccagaa
gactcagcaa acaaggtaga cagtgtctac caccatagtg
gccataccaa agaggctacc gattccttcc tgctacctgg
atccctgaag ttgccctggt ctctgcacct tctaaaccta
gttcttaaga gctttccatt acatgagctg tctcaaagcc
ctccaataaa ttctcagtgt aagcttctgt tgcttgtgga
cagaaaattc tgacagacct accctataag tgttactgtc
aggataacat gagaacgcac aacagtaagt ggtcactaag
tgttagctac ggttattttg cccaaggtag catggctagt
tgatgccggt tgatggggct taaacccagc tccctcatct
tccaggcctc tgtactccct attccactaa actacctctc
aggtttattt ttttaaattc ttactctgca agtacatagg
accacattta cctgggaaaa caagaataaa ggctgctctg
cattttttag aaactttttt gaaagggaga tgggaatgcc
tgcaccccca agtccagacc aacacaatgg ttaattgaga
tgaataataa aggaaagact gttctgggct tcccagaata
gcttggtcct taaattgtgg cacaaacaac ctcctgtcag
agccagcctc ctgccaggaa gaggggtagg agactagagg
ccgtgtgtgc agccttgccc tgaaggctag ggtgacaatt
tggaggctgt ccaaacaccc tggcctctag agctggcctg
tctatttgaa atgccggctc tgatgctaat cggcgaccct
caggcaagtt acttaacctt acatgcctca gttttctcat
ctggaaaatg agaaccctag gtttagggtt gttagaaaag
ttaaatgagt taagacaagt gcctgggaca cagtagcctc
ttgtgtgtgt ttatcattat gtcctcagca ggtcgtagaa
gcagcttctc aggtgtgagg ctggcgcgat tatctggagt
gggttgggtt ttctaggatg gaccccctgc tgcattttcc
tcattcatcc accagggctt aatggggaat caaggaatcc
atgtgtaact gtataataac tgtagccaca ctccaatgac
cacctactag ttgtccctgg cactgcttat acatatgtcc
atcaaatcaa tcctatgaag tagatactgt cttcatttta
tagatcagag acaattgggg ttcagagagc tgatgtgatt
ttcccagggt cacagagagt cccagattca ggcacaactc
ttgtattcca agacacaacc actacatgtc caaaggctgc
ccagagccac cgggcacggc aaattgtgac atatccctaa
agaggctgag cacctggtca ggatctgatg gctgacagtg
tgtccagatg cagagctgga gtgggggagg ggaagggggg
ctccttggga cagagaaggc tttctgtgct ttctctgaag
ggagcagtct gaggaccaag ggaacccggc aaacagcacc
tcaggtactc caggccctcc tggctggaga gggctgtggc
aatggaaaat tagtgccaac tgcaatgagt cagcctcggt
taaatagaga gtgaagaatg ctggacagga acctccaccc
tcatqtcaca tttcttcagt gtgacccttc tcgcccctct
cctcctgaca gcggaacaat gactgccccg ataggtgagg
ctggaggaag aatcagtcct gtccttggca agctcttcac
tatgacagta aaggctctct gcctgctgcc aaggcctqtg
actttctaac ctggcctcac gctgggtaag cttaaggtag
aggtgcagga ttagcaagcc cacctggcta ccaggccgac
agctacatct ttcaactgac cctgatcaac gaagagggac
ttgtgtctct cagttggttc caaatgaaac cagggagcag
gggcgttagg aagctccaac aggatggtac ttaatggggc
atttgagtgg agaggtaggt gacatagtgc tttggagccc
agggagggaa aggttctgct gaagttgaat tcaagactgt
tctttcatca caaacttgag tttcctggac atttgtttgc
agaaacaacc gtagggtttt gccttaacct cgtgggttta
ttattacctc atagggactt tgcctcctga cagcagttta
tgggtgttca ttgtggcact tgagttttct tgcatacttg
ttagagaaac caagtttgtc atcaacttct tatttaaccc
cctggctata acttcatgga ttatgttata attaagccat
ccagagtaaa atctgtttag attatcttgg agtaaggggg
aaaaaatctg taattttttc tcctcaacta gatatataca
taaaaaatga ttgtattgct tcatttaaaa aatataacgc
aaaatctctt ttccttctaa aaaaaaaaaa aaaaaa

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of an Homo sapiens cofilin 2 (CFL2), transcript variant 1, mRNA having NCBI Reference Sequence:

NM_021914.8 (SEQ ID NO: 53):
ccctttcgct tccacgtcca aaccccttta agaaggatga
atgggcagga tgagttagac tccttcgctg tatcgtctac
tgattcttaa aatgtgacaa atctgattgg acgacttaca
tggcttctgg agttacagtg aatgatgaag tcatcaaagt
ttttaatgat atgaaagtaa ggaaatcttc tacacaagag
gagatcaaaa agagaaagaa agcagttctc ttctgtttaa
gcgatgacaa aagacaaata attgtagagg aagcaaagca
gatcttggtg ggtgacattg gtgatactgt agaggacccc
tacacatctt ttctgaagtt gctacctctg aatgattgcc
gatatgcttt gtacgatgcc acatacgaaa caaaagagtc
taagaaagaa gacctagtat ttatattctg ggctcctgaa
agtgcacctt taaaaagcaa gatgatttat gctagctcta
aagatgccat taaaaagaaa tttacaggta ttaaacatga
gtggcaagta aatggcttgg atgatattaa ggaccgttcg
acacttggag agaaattggg aggcaatgta gtagtttcac
ttgaaggaaa accattataa aatgacagtc aagtgccatc
tggatcttaa ggagcttcca tttctccagc tcagtccatt
ggaatagtat taggttttgg ttttttgttg tatttccccc
tttccactgg gcccttccaa cacaatgaat gaaggaaata
tcatttattt aagcagccta tcagtgattg ccattagact
gttgaatact gttactttta tatagaaccc aaggaatgcc
ttcctgtcat attttagcca aaacaactgg ttatatgcct
cccttgcagc aagcactaca atgtatgtga tcgtcaatgt
gaatagctta gaatactgca aaggataagc taattgaatg
ccttgaaagt attatccact ggtcagatgg tcaacttttt
tcagtattat ttatagttgg cacttgattg cagttctgtg
aggcttgagc attcatacac ctcacctgcc ttggcaagcc
tattttagtg atatggcagc acggatataa cactatgcat
taaaagcact ttttgtaata agtttaatat cctaaaagga
atgccaatta agttttgtta actgtgtcat caacttatcc
tagtacctca gtgttcattc ctgttacctg catatcttct
taaaagaaat agctgttatt aatgcctttt tgttttccat
tgagtgtaca ctactgaata agtgtaggag ttttatgttt
accatgtgag tcctgcaaca ctaaagatat tttgaatatc
agtcatgatg gcaatttctg tataaaagag ccttaaatgg
aacattgttt tgagatcaaa ctccccaccc tcacaaaaat
ggccacgttg caataaaaat tgtggcatat tacagaacgt
tgccttgttt tccttggaaa ttttgcaaaa tgttatgtga
aacaacttct agggtaaaaa cagctattac taatctctgc
actggtcatt tgagaatttt ttttgtacag cattcatgtg
tgatattttc cagatttgtt ggatctattt ggtttaaaaa
gtattctatc ttaaggccaa ctaatataaa ataccattgt
taaagaatgg tacttttata aacattagtg tatttatttc
ctatgtgtta atatgaagat cagaaattat tttttgcact
ttggcataaa tacttttcaa tatctgattt gttctctgga
taaattagca tagttatttt tttattcaca tttacatttc
taagtagttg tatagtagaa gcaggaagct cttattgctt
atttggtcgt aatgaaaata atttgtaaaa tgtcctttaa
aagtttaatg atacttctga tctttcggaa cagtcatttc
acctactatt tctgaatata ttttgcaaat tgaattggaa
taggaattga taatagcagt cttaaacatt agtagtggga
tttggctatg gtccagactg tgctccttat agagaatttg
atctgctcag tgtgagcggt ttgctgttag ccagggctat
ttatggcaaa cacatgcttt tgtatcttgt catagttatc
cacaaatggc aaaactggac ttgattctac tggtatgcaa
aacaggcatg ctagtaagca gtcagtcgtg gctcagaact
taaccccata gctcagagga atgcttttag cagaaaacag
gaaagaaaat atcccttaaa aatttttttt gaatgtgtgg
aagtaatttt agtataatta gattttttcc atatttttga
aagatttttc agatgtgaac attaaaaata gggattaaat
gtctaggctt ccatttaaaa ttatatgaat ggtttgggat
ctttttgcac tgagcaattt tatttcaggc ttccagctgt
ccctgtgagt tatcctggac atttcgatgg tttttggtaa
ggccaaactc tgataagcaa aacagagaat actgacgtat
acttaaccat atgtgtaact gatacttggc accatggaat
ttttcattga gttatttcct cattctttta aaaaataagg
gactataaat cagttatgta gtatcttttg tttttgtagc
tgattcctta actttcttgt atgcctctag taatttcaga
gattaaatat tgctttaaac tgtgatactt tgatttgcta
gattgacaaa actgatacta atataattaa gttcatcttt
gaaatacatc tttgtgcgta gagccaaaaa aagagataaa
attaataata gttcacttgt tatttgagat taatttggca
tttgaaatga tcattttatt ttacaatcat ttataatgaa
tcaatgttcc agttagcttt aaaaggtata cggtgctaat
tagtaaaata ttgaaggcaa tattttactg ctagcttgca
aagttatgag agtttaaaaa ataaaatata tgaaaatatg
taaagctgtt gagatgtgtt tacttatact tcagaacatt
aaaagtttaa aaactggtat ttcagatcaa agaggcatca
ttatctttat tctgtttttc aggattttag ttggtaatac
tttctcattt atcaataaca attttcttta aaaactgagt
gttacagcaa ttaattttag cattttcaga caaatgggaa
cagttggcat gtgtcccaaa ctggctatca gctgttgttt
tccatcatta tctaaaatag tgtggccagc attgtgtatt
gaaatgtgcc tttttcgtac attggaagag aagcctctta
ctgggtttga gtttctctga tacagaacat ttgtagcagc
taatttatgg aatctggcaa ataagctttg ggaggaaatt
cttttaaaag tgttttcttg ttaaggaata atctcaacaa
tggtaaatca tcaagaggtt gagaagactc ttaggtttgc
cactcagaaa aaacagtctc attttgctct tcataggtcc
tgtttttgct tgcacattta atagtaggtg gagatgcaaa
acaacgtgaa tgttcatttt tttatgaata gtattaaagc
tactagaggt gggcaggcag gtcccagtaa atgttgcctt
tttttttttt tttttttgag acagggtctc actctgttgc
ccaggctgga gtgcagtgtc aaaatttcag ctcacttcaa
actcaacctt cccagctcag atgatcctcc cacctcagcc
tcccaagtag ctgggactac aggcatgcac caccatgcct
aatttttttt tttttttttt ttgtatctct tttgtagaga
ctgagtttgg ccatgttgcc taggctgatc tccaactcct
gggctcaagc tgtcctctga cctccgcctc ccaaaagtgc
taggattgca tgcatgagcc accacttcaa aagctcaaat
actgcttttg aagacagcat gagtttatgg atgttctgtc
tcctttcagc ccataaagaa gtctgaggct tttcttttgc
tctggaacat ccaactaatt gctttatttc catgtagaag
tagaggaatc atccctccac ttaaatttcc tctcataatg
caatttggca aattaaatct ctaaatctca gttttttaac
ccatagtatg ggaacggtaa tatctgttta ccatgtttcc
tgagtactaa atatggaaat ggtttttgaa aacaggaaaa
tgctatgtaa atgcaataat ctgtttaaac tattcattct
taattactgt atgtaagtag ataaatatta aatgtttttg
ttaaaagatg ta

a different transcript variant of the gene, or a sequence with at least 80%, 82%, 85%, 86%, 88% 90%. 92%, 94%, 95%, 97%, 98% or 99% nucleic acid sequence identity thereto.

In embodiments, the nucleic acid inhibits expression of treslin isoform 1 [Homo sapiens] mRNA having NCBI Reference Sequence:

```
NP_001294954.1 (SEQ ID NO: 54):
      1 atggcatgt gtcacaaagt aatgctgctg ctggacaccg cgggcggcgc cgcccgccac
    61 agccgggtcc gggggccgc cctgcgcctc ctcacctatc tgagttgccg attcggcctg
  121 gccagggtcc actgggcctt caagttcttt gactcgcagg gggcgcggag ccggccgtcc
  181 cgcgtgtctg acttccgcga gctggggtcc cgctcgtggg aggactttga ggaggagctg
  241 gaggccaggc tcgaggatcg cgcccacctg cccggcccgg cgcccagggc cacccacacg
  301 cacggcgccc tgatggagac gctgctagac taccagtggg accggcccga gatcacgtcg
  361 cccacgaagc cgatcctgcg gagcagcggg aggagactgc tggacgtgga gagcgaggcc
  421 aaggaggccg aggccgcgct cgggggcttg gtgaacgccg tcttcctcct ggccccctgt
  481 ccgcactcgc agagggagct gctgcagttc gtgtctgggt gcgaggccca ggcccagcgc
  541 ctgccgccca cccctaagca ggtgatggag aagttgttgc ccaagagagt ccgggaagtc
  601 atggtcgccc gaaaaatcac cttctactgg gtggatacca ccgaatggtc taagttgtgg
  661 gaatccccag accaccttgg atactggact gtttgtgaac tgctccacca cggaggtggc
  721 actgtcttgc catctgaatc tttcagctgg gattttgctc aagctgggga aatgctgctc
  781 aggagtggaa taaagctgtc aagtgaacct catctttctc cgtggatttc aatgctgcca
  841 actgatgcca ctttaaaccg tttgctctac aattctcctg agtatgaggc ctcgtttcca
  901 cgaatggaag gaatgttatt tctccctgtt gaaggcaaag agattcaaga aacatggaca
  961 gtcaccctag agcccttggc catgcatcag agacattttc agaaaccagt cagaattttt
1021 ctaaaaggct cagtggccca gtggtctctc ccaacgagca gcactttggg cactgacagc
1081 tggatgctag gaagtccaga ggagagcaca gcaactcaaa ggctgttatt tcagcagttg
1141 gtaagcaggc tgactgctga agagttacac ctggttgctg atgtggaccc tggtgaaggc
1201 cggcccccca tcactggagt tatttcccca ctctctgcca gtgctatgat cctcactgtg
1261 tgccgcacca aggaggctga atttcaacga catgttctcc aaacagctgt ggctgacagc
1321 ccccgggaca cagcttccct tttctcagat gttgtggata gtatattgaa tcagactcat
1381 gattcgcttg cagatactgc ttctgctgct tctcctgttc cagagtgggc ccagcaggag
1441 cttggccaca ccactccctg gagtccagct gttgtggaaa agtggtttcc tttctgtaac
1501 atcagtggtg ccagttccga tttgatggag tcatttgggt tactacaggc tgcctcagct
1561 aataaggaag agtcttccaa aactgaaggc gaattaatac attgccttgc cgagctctac
1621 cagagaaaat ctcgtgaaga atccactata gctcatcaag aagacagcaa aaagaaacga
1681 ggggtccctc gtactccagt gagacagaag atgaatacca tgtgccgttc cttaaagatg
1741 ttgaatgtcg caaggctgaa tgtgaaggcc cagaagttac atccagatgg cagtccggat
1801 gtggctgggg agaaaggaat ccaaaagata cctagtggga gaacagtgga taaattggaa
1861 gacagaggaa gaacactaag aagttctaaa cctaaagatt ttaaaactga ggaagagctg
1921 ctatcatata tacgtgaaaa ttaccaaaag actgtggcca caggagaaat catgttgtat
1981 gcatgtgctc gaaacatgat ctcaaccgtt aaaatgttcc taaaatcaaa aggcaccaag
2041 gaattagaag tgaactgcct gaatcaagta aaaagtagtc tcttaaaaac tagtaaaagt
2101 cttcgacaga atctaggaaa aaaactggat aaggaagaca aagttagaga gtgccagctt
2161 caggtatttc ttcgtttgga gatgtgtctg caatgccctt caataaatga aagtacagat
2221 gatatggaac aagtagtgga ggaggtgaca gatttgctgc gcatggtgtg tttaactgag
2281 gattcagcgt acctagcaga gtttctggag gaaattttga gattgtatat tgactctatc
2341 ccaaagacac ttggaaatct ttacaacagc ctagggtttg tgattcctca gaagctggct
2401 ggtgtccttc ctacagattt tttcagtgat gactccatga cacaagagaa caaatcacca
2461 cttctttctg tgcctttttt gtcaagtgct cgtagatcag tgtcaggcag ccctgaatct
2521 gatgaactgc aggaacttcg taccagatca gccaagaaga gaaggaaaaa tgcattaata
2581 agacataaaa gcattgctga ggtttcacag aatcttcgac aaattgaaat tcctaaagtg
2641 tcaaagagag ctacgaaaaa agagaactct caccctgctc ctcagcagcc ttcccagcca
2701 gtgaaagata cagtgcaaga agtgaccaaa gttcgaagaa atcttttcaa ccaggaattg
2761 ctttcccctt caaagagatc actaaagcgg gggttgccta gaagccattc tgtgtcagct
2821 gtggatggtc tagaggataa acttgacaac ttcaagaaga acaaaggtta tcacaaactg
2881 ctgactaaga gtgtggccga gactccagtg cataagcaga tctccaaaag gctgctgcac
2941 agacaaatca agggcaggtc ctctgatcct ggtcctgata ttggtgttgt tgaagagtcc
3001 cctgaaaaag gagatgaaat aagtctgaga cgaagtcctc gaatcaagca gttgtcattt
3061 agcaggacac attctgcctc cttctattct gtgtctcagc cgaagtctcg aagtgtgcaa
3121 agagtccact ctttccagca agataagtca gaccaaagag aaaattctcc agtccaaagt
3181 attcggtctc ccaagagtct tctttttggg gcaatgtctg agatgatcag cccctcagaa
3241 aagggttcag ctcgaatgaa aaagcgttca agaaacactt tggattcgga ggtacctgca
3301 gcttaccaga ctcccaagaa gagtcaccag aaatctctga gcttttctaa aactacacca
3361 agaaggatct ctcatacacc acaaactccg ttgtatactc cagaaaggct gcagaagtcc
3421 cctgcaaaaa tgacccctac aaagcaggca gcttttaagg agtccttaaa agactcctcc
3481 tcacccggcc atgactcacc attggattca aaaatcactc ctcaaaaacg acatacccag
3541 gcaggagaag gtacctctct tgaaacgaag acaccaagaa ctcctaagag gcaaggtact
3601 cagccgcctg ggtttttgcc aaactgtact tggccacatt cagtgaattc cagtccagaa
3661 agcccctcct gtccagcccc tccaacttca tcgactgccc agcccaggag agagtgtctc
3721 actcccatca gagaccctct cagaacacct ccgagagcag cagccttcat gggcacgcct
3781 cagaatcaaa cacaccaaca gccccatgtc ctcagagctg ctcgggcaga ggaaccagcc
3841 cagaaactaa aggataaagc tatcaaaact ccaaaaagac cagggaattc aactgtgact
3901 tcttccccac ctgttacgcc aaagaaactg tttacctctc ctttatgtga tgtctccaag
3961 aagagtccat ttaggaaatc taaaatagag tgtccttccc caggagaact ggatcagaaa
4021 gagccccaga tgtcacccag cgtagctgca tctctctcct gccctgttcc ctcaactccc
4081 cctgaactct cacagagagc tacattggac accgtccctc ctccaccccc ttctaaagtt
4141 gggaaacggt gtagaaagac ctctgatccc agaaggagca tcgtggagtg tcagcctgat
4201 gcctccgcta ctcctggggt tggcacagct gacagcccag ctgcccccac agactctaga
4261 gatgaccaga agggactgag cctctctcct cagtctcctc ctgaaagacg gggctaccca
4321 ggccctggtc tcaggagtga ttggcatgca tcctctcctc tgctcattac aagtgacaca
4381 gagcatgtca ctctcctcag tgaagccgaa caccatggca ttggtgactt gaaaagtaac
4441 gtcttatcag tggaagaggg tgaggggcta aggacagcag atgctgagaa gtcttctctg
4501 tctcaccctg ggattccccc atctcctcct tcctgtgggc ctggctctcc tctgatgcct
4561 tcccgtgacg tgcactgtac cacagatggg agacagtgcc aggcttcggc acaactagac
4621 aacctgccag catcagcttg gcattccaca gactctgcca gcccacagac ctatgaggtt
4681 gagctggaga tgcaagcttc tggccttccc aaacttcgaa ttaagaagat agaccccagc
4741 tcttcattag aggctgagcc cctcagcaag gaggagagct ctctgggaga agagagcttc
4801 ctccctgctc tcagcatgcc cagggccagc aggtccttaa gcaaacctga acccacctat
4861 gtgtcacccc cctgcccccg cctctcccac agcacacctg gcaagagcag ggggcaaacc
4921 tacatctgcc aggcctgtac ccccacccac ggcccttcta gtaccccctc tccatttcaa
4981 acagatgggg ttccttggac accatccccc aagcacagtg ggaagacaac tccagacata
5041 attaaagact ggcccaggag gaagagggcg gtgggctgtg gcgccggctc ctcttccggg
5101 aggggcgagg tcggtgcaga ccttcctggg agcctgtcac tgcttgagtc agagggcaag
5161 gaccacggcc ttgaactcag catccacagg acgcccatct tggaggattt tgagctcgag
5221 ggagtgtgcc agctcccaga ccagtcgcct cccaggaaca gcatgcctaa ggccgaggaa
5281 gcctcttcct ggggacagtt tgggttgagt tccaggaaga gagtcctgtt ggccaaggaa
5341 gaagctgacc gtggagccaa aaggatctgt gacctgagag aagattcaga agttagtaag
5401 agtaaagagg ggtctccaag ttggagtgca tggcagctac cctccacggg agacgaagag
5461 gtgtttgttt ccggctccac cccacctccc agctgtgccg tgcggagctg cctctctgcc
5521 agtgccctcc aggctctgac ccagtctccg ctgctgttcc aggggaaaac accttcctct
5581 cagagcaaag accccagaga tgaggatgtg gatgttcttc cctccactgt agaagactct
5641 cctttcagtc gcgctttctc caggaggcgc cccatcagca gaacttatac acggaagaag
5701 ctcatgggaa cctggctgga ggacttatag ccacaaacat tactgagccc aaaagatcaa
5761 ggagtcagcc aggaccctgt ggacataaag aagttggatg cctggtccca agcctctttt
5821 gccatggtca gtgttcagat tgccattaga atgccttagg gttttctaat tccccttatg
5881 gatccaatcc atctcctggc cctgcccctt gttggggaag ttgcaggagg agaggtggat
5941 ggcaatgtga ttggtgctat aactcaggca gcctgggagt caggaaccca gacaaggaat
6001 cccattccag cctcacccca accatgacct tggcaagtca gggggccact ctgcctcatt
6061 tatgcaaatg gagaaaggcg ccctccctgg ggtcccttga gctgctgtaa ggctgggctg
6121 ctgcgacaca ggcagcgctt tgtaaactgt gaagccatat acgtgaaact gaagagtgca
6181 ttgggcagtg gaagctattt tttgccttcc ctgtgtaaca gtaaaatcat ctctagtgac
6241 tgagcactca gtacattttt gtttaatgtt gggcctgagg ttaactgtga ccatggtcca
6301 gcttgagtgg cttctggagc agccacattt tcaaggactg tccaagagcc agccagttca
6361 gggctcaggc ctcacccatt gcccactcct ggggagacca tcacctggct catcgtttcc
6421 accaagagtg ccccacagga gtgccccaca gacccgctgg accagcctgc tgcgggtcct
6481 ggccaggggt ctggctaacg gtgagggctg actctgaact gtctctcagt ctccagaaag
6541 tgttcaagcc tgttgtgttc ccaaatctga ttcctcctat tgtcttgtaa atcaaactct
6601 aagtgaaaac ttcccatttg tcccttcaaa gatttttttt tattaaatgg ttttttaaga
6661 tcctaaaaaa aaaaaaaaaa aaaaa
```

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of Homo sapiens macrophage immunometabolism regulator (MACIR), transcript variant 1, mRNA having NCBI Reference Sequence:

```
NM_001316968.2 (SEQ ID NO: 55):
      1 cccttggccg ctgttcccgc accgcggggc agcgggcctg gaggcccctt tgagaagtag
    61 ctttccccgg ccggcggcac ctttggctgc gtgcccggcc gcgctcaggg tgcgactgcc
  121 cgggtcagat agcacctcag ggcgagcccc ggcggtctga tctcgccgac cctcctcgtc
  181 ctggttgcaa cccgcgtgcg aggccgcccg cgccctccga gtgtctgccg gtgcagtggg
  241 ggtggactgg ccggctgtgg ccgtgtgtgc gcgtgtggat tggggcccgg tccgagccag
  301 aagcttaagc ggcagatgtc gggcattgcc accctcgccc cacctgtcgc gggtggactt
  361 tggggcagta cctggagtag aacagaaaaa ttattatgtc tgtgttccct tgggactcat
  421 tggaaattgt acagtgacat cttctgggat ttagtctgga ttgtgcagac tggtgcttaa
  481 aatggaagtc gatattaatg gagagtctag aagtaccctg accaccttgc ccttccctgg
  541 ggctgaggcc aactccccgg gaaaggcgga ggcagagaag ccccgctgct ccagcacacc
  601 ctgctccccg atgcggagga ccgtgtcagg ctaccagatc ctacacatgg actctaacta
  661 tttggttggc ttcacgactg gcgaggaact cctgaagtta gctcagaagt gcacaggagg
  721 tgaagagagc aaagcagaag ccatgccatc cttacgctcc aaacagctag atgcaggact
  781 tgcccgttcc tctcgtttgt ataaaaccag aagtaggtac taccagccat acgagattcc
  841 agctgtcaat ggcaggaggc gaaggcggat gccaagctca ggagacaagt gcactaaatc
  901 tttaccttat gaaccttaca aggccctcca tgggcctctg cctctttgtc ttcttaaagg
  961 taagagggct cactccaaat ctctggacta cctcaatcta gataaaatga tcaaggagcc
1021 agctgataca gaagtgctac agtaccagct tcaacaccta accctccgag gggaccgtgt
1081 gtttgctagg aataatacat gaatgacttg gagagagctt aaaccaattt aggtcagcct
1141 acgcttggct agaaaaaacc cactgctgta ctctgtacat gactcttcac actatagatg
1201 gttatatcag ctaagtgttc ctggaacata aaaattgttt gggtcaaatt tgaatacagg
1261 aatgaaatca caggtacttg ggggggggat atcattctag agcacgcaac tgcaaagaaa
1321 acagaatgtt gactgttagt ttgtatagct ttttagctag gaaaaaaagg ctttggtaca
1381 gtaatttcat ctttatgatt ctgacactca aattgggaat gttacctgct tggttgttgc
1441 tgacttgata tgattcatta gaaatttata tcttcagtac tcaagtactt cttgaatctc
1501 tgtattttac tataaaatgt atgtaatgat ttgttttatg aaatttagaa cttgaacatt
1561 gctgaattgg accacttttt atttttaaat attgagttta aatattttat aactggtttt
1621 gcactgaaaa aattaacatt tcagattgac aagagagtaa tctttcttca cttgcctcaa
1681 taatgttatt gagcaatgaa ttttttattt ccgcatggaa agttattgat ctctatggct
1741 gtaaaatatt tctttatagc gttattaaag tgtgtcttaa taaaattaaa tttgggatac
1801 aaagtattta ttttacaatg ggtgggcggg ggaaactttt ccagaaagtt tccaatatga
1861 cgttttcata agttgaaaaa actctcctta gtgcttattt tctaacttaa aattcacctg
1921 gaactttaaa tcggaaagga ttcttttaat tgtggattat aggcataata ctgtttgcat
1981 ctgaattttc tgtaagtgaa taatagttta atagaggaac tcatgatttg tactattgaa
2041 tgattaaact aagtatgaag tgataccatt cagcatggca tcaggtcatt gcagttttag
2101 ttctgtgtaa cacaagcact cactgaaatt ccagtttcta ggattagtgt aggagcctaa
2161 cgtgcttcta ctgttttaat gggttaatcc tggattactt aacaatttat gtcaattgca
2221 ctggtttaat ttgttgctaa agaaataatg ccctgggttt agtaacaaat acagctcaac
2281 tattcttgaa tatattttga aaaaaaaatg tatgtaactt accttttgta aacgttccat
2341 ttcttttttc cctcattttt gactcttaaa ggtgcaattt attactgaat tgggatttct
2401 ggcagcacag aactgctttt tattttgggg tctgtgagtt tcttaggtat tagcaatctt
2461 gcttataaaa taagaacacc ttttaattaa tgagtgggtc attcctggtg caattgtgat
2521 ttttctttag ccagaatgaa tggcaaactc tatttagagc aaagtaagta ttagaaaacc
2581 ctaggaactc ttaatcaacg tttattacac tttcattaag gcaaactacg tgaaagagcc
2641 ttggggaagt tggcccatat cttactaagt tgatcagatt tctcgttggg ctggaaatgt
2701 ttcgctgttg tatattttaa agtaaattgc acctttgtaa catattgtat tgacgaatga
2761 tcactaagat tagctatatc tatacagtca ttagtttgac aagaaataga atcctgtcag
2821 atgccaaaga gtgggatttt tatgtttaat gattaaacac cattatttat tgacaattta
2881 ccctgtggaa ctgtattatt tctaactatg aaataaaggg gtgatgtaaa cacacattgt
2941 tgtgtggtgc tttaaactag gtccactatc aacaggctac ttactgttca agaattccac
3001 tgaagcactt attttaaggc cctatttttc ttaaacaaaa cagtgacaac aacaatcaaa
3061 ccatttactt ttgatgctca ttggcatttt atgataaaag atgtattcat ggcaatgata
3121 tgtattcacc ctattaggaa acacaactgg ttacctatga gacctgttct gtccgtgtgc
3181 ctacgttcct taataatagc taaataaaaa tttgtagctt tt
```

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of Homo sapiens gasdermin B (GSDMB), transcript variant 1, mRNA having NCBI Reference Sequence:

(SEQ ID NO: 56):
NM_001042471.2
attattttag cttcctgaga ttcagaggcc aggaactgtg
cagagatctg tggggattct cacaacttcc atttctggtg
aacagctgag gtcagagagg agttggtcca ggcgcaatgt
tcagcgtatt tgaggaaatc acaagaattg tagttaagga
gatggatgct ggaggggata tgattgccgt tagaagcctt
gttgatgctg atagattccg ctgcttccat ctggtggggg
agaagagaac tttctttgga tgccggcact acacaacagg
cctcaccctg atggacattc tggacacaga tggggacaag
tcgttagatg aactggattc tgggctccaa ggtcaaaagg
ctgagtttca aattctggat aatgtagact caacgggaga
gttgatagtg agattaccca aagaaataac aatttcaggc
agtttccagg gcttccacca tcagaaaatc aagatatcgg
agaaccggat atcccagcag tatctggcta cccttgaaaa
caggaagctg aagagggaac tacccttttc attccgatca
attaatacga gagaaaacct gtatctggtg acagaaactc
tggagacggt aaaggaggaa accctgaaaa gcgaccggca
atataaattt tggagccaga tctctcaggg ccatctcagc
tataaacaca agggccaaag ggaagtgacc atccccccaa
atcgggtcct gagctatcga gtaaagcagc ttgtcttccc
caacaaggag acgatgaaga aggatggtgc ttcatcctgt
ttaggaaagt ctttgggttc ggaggattcc agaaacatga
aggagaagtt ggaggacatg gagagtgtcc tcaaggacct
gacagaggag aagagaaaag atgtgctaaa ctccctcgct
aagtgcctcg gcaaggagga tattcggcag gatctagagc
aaagagtatc tgaggtcctg atttccgggg agctacacat
ggaggaccca gacaagcctc tcctaagcag cctttttaat
gctgctgggg tcttggtaga agcgcgtgca aaagccattc
tggacttcct ggatgccctg ctagagctgt ctgaagagca
gcagtttgtg gctgaggccc tggagaaggg gacccttcct
ctgttgaagg accaggtgaa atctgtcatg gagcagaact
gggatgagct ggccagcagt cctcctgaca tggactatga
ccctgaggca cgaattctct gtgcgctgta tgttcttgtc
tctatcctgc tggagctggc tgaggggcct acctctgtct
cttcctaact acaaaagccc tttctcccca caagcctctg
ggttttccct ttaccagtct gtcctcactg ccatcgccac
taccatcctg tcaccagtgg gacctcttta aaacaagcag
ccaaccattc tttgatgtat cccattcgct ccatgttaac
atccaaaacc agcctggatt tcatacatgg acttctgatt
aaaagtggca ggttgtgcat gttaa

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of Homo sapiens bromodomain containing 4 (BRD4), transcript variant long, mRNA having NCBI Reference Sequence:

NM_058243.3 (SEQ ID NO: 57):
attctttgga atactactgc tagaagtctg acttaagacc
cagcttatgg gccacatggc acccagctgc ttctgcagag
aaggcaggcc actgatgggt acagcaaagt gtggtgctgc
tggccaagcc aaagacccgt gtaggatgac tgggcctctg
ccccttgtgg gtgttgccac tgtgcttgag tgcctggtga
agaatgtgat gggatcacta gcatgtctgc ggagagcggc
cctgggacga gattgagaaa tctgccagta atgggggatg
gactagaaac ttcccaaatg tctacaacac aggcccaggc
ccaaccccag ccagccaacg cagccagcac caaccccccg
cccccagaga cctccaaccc taacaagccc aagaggcaga
ccaaccaact gcaatacctg ctcagagtgg tgctcaagac
actatggaaa caccagtttg catggccttt ccagcagcct
gtggatgccg tcaagctgaa cctccctgat tactataaga
tcattaaaac gcctatggat atgggaacaa taaagaagcg
cttggaaaac aactattact ggaatgctca ggaatgtatc
caggacttca acactatgtt tacaaattgt tacatctaca
acaagcctgg agatgacata gtcttaatgg cagaagctct
ggaaaagctc ttcttgcaaa aaataaatga gctacccaca
gaagaaaccg agatcatgat agtccaggca aaaggaagag
gacgtgggag gaaagaaaca gggacagcaa aacctggcgt
ttccacggta ccaaacacaa ctcaagcatc gactcctccg
cagacccaga cccctcagcc gaatcctcct cctgtgcagg
ccacgcctca ccccttccct gccgtcaccc cggacctcat
cgtccagacc cctgtcatga cagtggtgcc tccccagcca
ctgcagacgc ccccgccagt gcccccccag ccacaacccc
cacccgctcc agctccccag cccgtacaga gccacccacc
catcatcgcg gccaccccac agcctgtgaa gacaaagaag
ggagtgaaga ggaaagcaga caccaccacc cccaccacca
ttgaccccat tcacgagcca ccctcgctgc ccccggagcc
caagaccacc aagctgggcc agcggcggga gagcagccgg
cctgtgaaac ctccaaagaa ggacgtgccc gactctcagc
agcacccagc accagagaag agcagcaagg tctcggagca
gctcaagtgc tgcagcggca tcctcaagga gatgtttgcc
aagaagcacg ccgcctacgc ctggcccttc tacaagcctg
tggacgtgga ggcactgggc ctacacgact actgtgacat
catcaagcac cccatggaca tgagcacaat caagtctaaa
ctggaggccc gtgagtaccg tgatgctcag gagtttggtg
ctgacgtccg attgatgttc tccaactgct ataagtacaa
ccctcctgac catgaggtgg tggccatggc ccgcaagctc
caggatgtgt tcgaaatgcg ctttgccaag atgccggacg
agcctgagga gccagtggtg gccgtgtcct ccccggcagt
gccccctccc accaaggttg tggccccgcc ctcatccagc
gacagcagca gcgatagctc ctcggacagt gacagttcga
ctgatgactc tgaggaggag cgagcccagc ggctggctga
gctccaggag cagctcaaag ccgtgcacga gcagcttgca
gccctctctc agccccagca gaacaaacca aagaaaaagg
agaaagacaa gaaggaaaag aaaaaagaaa agcacaaaag
gaaagaggaa gtggaagaga ataaaaaaag caaagccaag
gaacctcctc ctaaaaagac gaagaaaaat aatagcagca
acagcaatgt gagcaagaag gagccagcgc ccatgaagag
caagccccct cccacgtatg agtcggagga agaggacaag
tgcaagccta tgtcctatga ggagaagcgg cagctcagct
tggacatcaa caagctcccc ggcgagaagc tgggccgcgt
ggtgcacatc atccagtcac gggagccctc cctgaagaat
tccaaccccg acgagattga aatcgacttt gagaccctga
agccgtccac actgcgtgag ctggagcgct atgtcacctc
ctgtttgcgg aagaaaagga aacctcaagc tgagaaagtt
gatgtgattg ccggctcctc caagatgaag ggcttctcgt
cctcagagtc ggagagctcc agtgagtcca gctcctctga
cagcgaagac tccgaaacag agatggctcc gaagtcaaaa
aagaaggggc accccgggag ggagcagaag aagcaccatc
atcaccacca tcagcagatg cagcaggccc cggctcctgt
gccccagcag ccgcccccgc ctccccagca gcccccaccg
cctccacctc cgcagcagca acagcagccg ccacccccgc
ctcccccacc ctccatgccg cagcaggcag ccccggcgat
gaagtcctcg cccccaccct tcattgccac ccaggtgccc
gtcctggagc cccagctccc aggcagcgtc tttgacccca
tcggccactt cacccagccc atcctgcacc tgccgcagcc
tgagctgccc cctcacctgc cccagccgcc tgagcacagc
actccacccc atctcaacca gcacgcagtg gtctctcctc
cagctttgca caacgcacta ccccagcagc catcacggcc
cagcaaccga gccgctgccc tgcctcccaa gcccgcccgg
cccccagccg tgtcaccagc cttgacccaa acacccctgc
tcccacagcc ccccatggcc caaccccccc aagtgctgct
ggaggatgaa gagccacctg ccccacccct cacctccatg
cagatgcagc tgtacctgca gcagctgcag aaggtgcagc
cccctacgcc gctactccct tccgtgaagg tgcagtccca
gcccccaccc cccctgccgc ccccacccca cccctctgtg
cagcagcagc tgcagcagca gccgccacca cccccaccac
cccagcccca gcctccaccc cagcagcagc atcagccccc
tccacggccc gtgcacttgc agcccatgca gttttccacc
cacatccaac agcccccgcc accccagggc cagcagcccc
cccatccgcc cccaggccag cagccacccc cgccgcagcc
tgccaagcct cagcaagtca tccagcacca ccattcaccc
cggcaccaca agtcggaccc ctactcaacc ggtcacctcc
gcgaagcccc ctccccgctt atgatacatt ccccccagat
gtcacagttc cagagcctga cccaccagtc tccaccccag
caaaacgtcc agcctaagaa acaggagctg cgtgctgcct
ccgtggtcca gccccagccc ctcgtggtgg tgaaggagga
gaagatccac tcacccatca tccgcagcga gcccttcagc
ccctcgctgc ggccggagcc ccccaagcac ccggagagca
tcaaggcccc cgtccacctg ccccagcggc cggaaatgaa
gcctgtggat gtcgggaggc ctgtgatccg gcccccagag
cagaacgcac cgccaccagg ggcccctgac aaggacaaac
agaaacagga gccgaagact ccagttgcgc ccaaaaagga
cctgaaaatc aagaacatgg gctcctgggc cagcctagtg
cagaagcatc cgaccacccc ctcctccaca gccaagtcat
ccagcgacag cttcgagcag ttccgcagcg ccgctcggga
gaaagaggag cgtgagaagg ccctgaaggc tcaggccgag
cacgctgaga aggagaagga gcggctgcgg caggagcgca
tgaggagccg agaggacgag gatgcgctgg agcaggcccg
gcgggcccat gaggaggcac gtcggcgcca ggagcagcag
cagcagcagc gccaggagca acagcagcag cagcaacagc
aagcagctgc ggtggctgcc gccgccaccc cacaggccca
gagctcccag ccccagtcca tgctggacca gcagagggag
ttggcccgga agcgggagca ggagcgaaga cgccgggaag
ccatggcagc taccattgac atgaatttcc agagtgatct
attgtcaata tttgaagaaa atcttttctg agcgcaccta
ggtggcttct gactttgatt ttctggcaaa acattgactt
tccatagtgt taggggcggt ggtggaggtg ggatcagcgg
ccaggggatg cctcagggcc tggccctcct gcatgctatg
cccggggcag gcctgacggg cagctgagga ttgcagagcc
tgtctgcctt acggccagtc ggacagacgt cccgccaccc
accacccctc acaggacgtc cgctcagcac acgccttgtt
acgagcaagt gccggctgga cccaagccct gcatccccac
atgcggggca gaggcccttc tctccgccaa atgtctacac
agtatacaca ggacatcgtt gctgccgccg tgactggttt
tctgtcccca agaacgtgac gttcgtgatg tcctgcccgc
cgggagtctt tccccacacc ccagccatcg ccgcccgctc
ccaggaggcc agggcaggcc tgcgtgggct ggaggcgggc
gaggccggcc caccccctcg ctggcactga ctttgccttg
aacagacccc ccgaccctcc cccacaagcc tttaattgag
agccgctctc tgtaagtgtt tgcttgtgca aaagggaata
gtgccgtgga ggtgtgtgtg tccatggcat ccggagcgag
gcgactgtcc tgcgtgggta gccctcggcc ggggagtgag
gccaccaacc aaagtcagtt ccttcccacc tgtgtttctg
tttcgttttt ttttttcttt tttttctata tatatttttt
gttgaattct attttatttt taattctctc ttctcctcca
gacacaatgg cactgcttat ctccgaaatg gtgtgatcgt
ctcctcattg agcagcggct gccaccgcgc tgtgggtagt
gtgtgaccgt ggctgtactg tatagtgaac atagttggca
tatctttgtt tgaagtttgt tggtgactcc accaaactgg
tgtgaaaaaa gaaaaaagct caaaaaaatc cacaaaaaga
caaaacacac aaaaaaaatc ctgcctatat tttactcagt
ttcaaacttt attagtctat ttttaattat aaaaccagaa
agctacaatt tcttttcttt cccctccacc cccccccccc
ccacccattt gttggctttt ttgtttttta atgtcagatc
tcttgagttg gtttttttgg ttttggtttt tgtttttgtt
tttgtttttt actgagaaag gaagggccaa gggatgaggt
gggaaccggg ccctgggggc gccacagact aaggcagaga
ctcccctacc tggcgcccag ccccaaccag ctggccgctc
ctgcccatgc tttttttttt tttttttttt aatttttata
attggagccc ctggtgaggt tacgcgtgcc atgagaaccc
actctacacc acgacgctgg tgcctcagtg ttggccaaac
tctggagtca ctgactggtt tgactttcat acggtgaata
tgcatttggt ctgtactgat catggaataa acacatctct
ctttttttaa tgctggcgtc tccctgacat ttctttgtga
accaactgtt gcctaggcta ggcccagggg accccctgga
ccccagacca cctctgtaca ggaactactg ccagggatta
cctagcccct ctcctgtgac ctgtccctgt ctgccctggg
cgggagccac gcagactcat agcaaccacc ccaagctgaa
gctgtgacgc agagcccggt acccatcctt gtggaccctg
gctgaggtgg agggtgtgct ctagcagaac cctggccaga
ctccagacag tattcttccc ctccacccta ctccatcctg
tcccccctac tccatcctgt ccccccactc accaaaggac
ttgggccact tctcccacct tgcctgcctc aacctaactc
ctcctttcat ttaagctcag ggttaaccag atactcttag
atataagtct acatccccca aaataggatc ctcacccccc
atgcacatac acacacattc ctgtccaaga aagcccacag
gtggctgctc tgcctgtgtg tccacttgtg tatgtacatg
ccccagccac aaggcacggg tgacgcccaa gaagagcccc
taagatgtaa gatacaagta tataatttat atgtatgcag
agacaaactg attgaaacat ttctagcact gtttattctc
ctacatcccc tctttttgac ctgaaaggtc ctttattgtc
tttggatctg ccaaacctcc ctgtgagggt gtggttcagg
catccagcca gaggtccccc cacttcttcg atgtccatgc
ccactcaggc actttctcat ataccactgt gccacttcaa
gtgtgacgta tttaggttct ttggggtttc ttccatctca
tcccacccac tcatttttgt tccttgtttt gttcagacac
ttccccagcc tgggtccaac tgctgagctg gatagttttc
tctgcagctt cctgctgaag atttaactca gccatggcct
tctccacacc tccctgatgg gtgtctctct acctgggttg
taattagaac tgggatctat ttaatttctc cagccatttc
ccaacccttc ccacagtact agaaatctta gtcctatacc
agagtaagag gtgtgtacaa gcccccactg tactgtatgc
acggatcgct tggccaataa ttatgtcagt gaatctgaga
cttgtattaa acactttaga catttgtaga agggaattcg
tagacttttc acttacatac gaaaggtttt tttttttttt
tgtgcagttc tcattgcaaa aataaacatt tgtactgagt
ataaagtta

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of Homo sapiens interferon induced protein with tetratricopeptide repeats 3 (IFIT3), transcript variant 1, mRNA having NCBI Reference Sequence:

NM_001549.6 (SEQ ID NO: 58):
gcagacagga agacttctga agaacaaatc agcctggtca
ccagcttttc ggaacagcag agacacagag ggcagtcatg
agtgaggtca ccaagaattc cctggagaaa atccttccac
agctgaaatg ccatttcacc tggaacttat tcaaggaaga
cagtgtctca agggatctag aagatagagt gtgtaaccag
attgaatttt taaacactga gttcaaagct acaatgtaca
acttgttggc ctacataaaa cacctagatg gtaacaacga
ggcagccctg gaatgcttac ggcaagctga agagttaatc
cagcaagaac atgctgacca agcagaaatc agaagtctag
tcacttgggg aaactacgcc tgggtctact atcacttggg
cagactctca gatgctcaga tttatgtaga taaggtgaaa
caaacctgca agaaattttc aaatccatac agtattgagt
attctgaact tgactgtgag gaagggtgga cacaactgaa
gtgtggaaga aatgaaaggg cgaaggtgtg ttttgagaag
gctctggaag aaaagcccaa caacccagaa ttctcctctg
gactggcaat tgcgatgtac catctggata atcacccaga
gaaacagttc tctactgatg ttttgaagca ggccattgag
ctgagtcctg ataaccaata cgtcaaggtt ctcttgggcc
tgaaactgca gaagatgaat aaagaagctg aaggagagca
gtttgttgaa gaagccttgg aaaagtctcc ttgccaaaca
gatgtcctcc gcagtgcagc caaattttac agaagaaaag
gtgacctaga caaagctatt gaactgtttc aacgggtgtt
ggaatccaca ccaaacaatg gctacctcta tcaccagatt
gggtgctgct acaaggcaaa agtaagacaa atgcagaata
caggagaatc tgaagctagt ggaaataaag agatgattga
agcactaaag caatatgcta tggactattc gaataaagct
cttgagaagg gactgaatcc tctgaatgca tactccgatc
tcgctgagtt cctggagacg gaatgttatc agacaccatt
caataaggaa gtccctgatg ctgaaaagca acaatcccat
cagcgctact gcaaccttca gaaatataat gggaagtctg
aagacactgc tgtgcaacat gctttagagg gtttgtccat
aagcaaaaaa tcaactgaca aggaagagat caaagaccaa
ccacagaatg tatctgaaaa tctgcttcca caaaatgcac
caaattattg gtatcttcaa ggattaattc ataagcagaa
tggagatctg ctgcaagcag ccaaatgtta tgagaaggaa
ctgggccgcc tgctaaggga tgccccttca ggcataggca
gtattttcct gtcagcatct gagcttgagg atggtagtga
ggaaatgggc cagggcgcag tcagctccag tcccagagag
ctcctctcta actcagagca actgaactga gacagaggag
gaaaacagag catcagaagc ctgcagtggt ggttgtgacg
ggtaggacga taggaagaca gggggcccca acctgggatt
gctgagcagg gaagctttgc atgttgctct aaggtacatt
tttaaagagt tgttttttgg ccgggcgcag tggctcatgc
ctgtaatccc agcactttgg gaggccgagg tgggcggatc
acgaggtctg gagtttgaga ccatcctggc taacacagtg
aaatcccgtc tctactaaaa atacaaaaaa ttagccaggc
gtggtggctg gcacctgtag tcccagctac ttgggaggct
gaggcaggag aatggcgtga acctggaagg aagaggttgc
agtgagccaa gattgcgccc ctgcactcca gcctgggcaa
cagagcaaga ctccatctca aaaaaaaaaa aaaaaaaaaa
aaagagttgt tttctcatgt tcattatagt tcattacagt
tacatagtcc gaaggtctta caactaatca ctggtagcaa
taaatgcttc aggcccacat gatgctgatt agttctcagt
tttcattcag ttcacaatat aaccaccatt cctgccctcc
ctgccaaggg tcataaatgg tgactgccta acaacaaaat
ttgcagtctc atctcatttt catccagact tctggaactc
aaagattaac ttttgactaa ccctggaata tctcttatct
cacttatagc ttcaggcatg tatttatatg tattcttgat
agcaatacca taatcaatgt gtattcctga tagtaatgct
acaataaatc caaacatttc aactctgtta

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of Homo sapiens opioid growth factor receptor (OGFR), mRNA having NCBI Reference Sequence:

NM_007346.4 (SEQ ID NO: 59):
agcgcgagcc ccgccgccgc cgagcatgga cgaccccgac
tgcgactcca cctgggagga ggacgaggag gatgcggagg
acgcggagga cgaggactgc gaggacggcg aggccgccgg
cgcgagggac gcggacgcag gggacgagga cgaggagtcg
gaggagccgc gggcggcgcg gcccagctcg ttccagtcca
gaatgacagg gtccagaaac tggcgagcca cgagggacat
gtgtaggtat cggcacaact atccggatct ggtggaacga
gactgcaatg gggacacgcc aaacctgagt ttctacagaa
atgagatccg cttcctgccc aacggctgtt tcattgagga
cattcttcag aactggacgg acaactatga cctccttgag
gacaatcact cctacatcca gtggctgttt cctctgcgag
aaccaggagt gaactggcat gccaagcccc tcacgctcag
ggaggtcgag gtgtttaaaa gctcccagga gatccaggag
cggcttgtcc gggcctacga gctcatgctg ggcttctacg
ggatccggct ggaggaccga ggcacgggca cggtgggccg
agcacagaac taccagaagc gcttccagaa cctgaactgg
cgcagccaca acaacctccg catcacacgc atcctcaagt
cgctgggtga gctgggcctc gagcacttcc aggcgccgct
ggtccgcttc ttcctggagg agacgctggt gcggcgggag
ctgccggggg tgcggcagag tgccctggac tacttcatgt
tcgccgtgcg ctgccgacac cagcgccgcc agctggtgca
cttcgcctgg gagcacttcc ggccccgctg caagttcgtc
tgggggcccc aagacaagct gcggaggttc aagcccagct
ctctgcccca tccgctcgag ggctccagga aggtggagga
ggaaggaagc cccggggacc ccgaccacga ggccagcacc
cagggtcgga cctgtgggcc agagcatagc aagggtgggg
gcagggtgga cgaggggccc cagccacgga gcgtggagcc
ccaggatgcg ggacccctgg agaggagcca gggggatgag
gcagggggcc acggggaaga taggccggag cccttaagcc
ccaaagagag caagaagagg aagctggagc tgagccggcg
ggagcagccg cccacagagc caggccctca gagtgcctca
gaggtggaga agatcgctct gaatttggag gggtgtgccc
tcagccaggg cagcctcagg acggggaccc aggaagtggg
cggtcaggac cctggggagg cagtgcagcc ctgccgccaa
cccctgggag ccagggtggc cgacaaggtg aggaagcgga
ggaaggtgga tgagggtgct ggggacagtg ctgcggtggc
cagtggtggt gcccagacct tggcccttgc cgggtcccct
gccccatcgg ggcaccccaa ggctggacac agtgagaacg
gggttgagga ggacacagaa ggtcgaacgg ggcccaaaga
aggtacccct gggagcccat cggagacccc aggccccagc
ccagcaggac ctgcagggga cgagccagcc gagagcccat
cggagacccc aggcccccgc ccagcaggac ctgcagggga
cgagccagcc gagagcccat cggagacccc aggcccccgc
ccggcaggac ctgcagggga cgagccagcc gagagcccat
cggagacccc aggccccagc ccggcaggac ctacaaggga
tgagccagcc gagagcccat cggagacccc aggcccccgc
ccggcaggac ctgcagggga cgagccagcc gagagcccat
cggagacccc aggcccccgc ccggcaggac ctgcagggga
cgagccagcc gagagcccat cggagacccc aggccccagc
ccggcaggac ctacaaggga tgagccagcc aaggcggggg
aggcagcaga gttgcaggac gcagaggtgg agtcttctgc
caagtctggg aagccttaag gaaaggagtg cccgtcggcg
tcttggtcct cctgtccctg ctgcaggggc tggggcctcc
ggagctgctg cgggctcccc tcaggctctg cttcgtgacc
cgtgacccat gacccacagt gctggcctcc tgtggggcca
ctatagcagc caccagaagc cgcgaggccc tcagggaagc
ccaaggcctg cagaagcctc ctggcctggc tgtgtcttcc
ccacccagct ctcccctgcg cccctgtctt tgtaaattga
cccttctgga gtggggggcg gcgggcaggg ctgcttttct
tagtctgata ccaagcaagg ccttttctga ataaattcat
ttgactttga

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of Homo sapiens short chain dehydrogenase/reductase family 39U member 1 (SDR39U1), transcript variant 1, mRNA having NCBI Reference Sequence:

NM_020195.3 (SEQ ID NO: 60):
agtcgctatg cgtgtgcttg tgggtggcgg gacaggcttc
attgggacag ccctaaccca gctgctgaat gccagaggcc
acgaagtgac gttggtctcc cgaaagcccg ggcccggccg
gatcacgtgg gatgagctcg ctgcatcggg gctgccgagc
tgcgatgccg ccgtcaacct ggccggagag aacatcctca
accctctccg aagatggaat gaaaccttcc aaaaagaggt
aatcggcagc cgcctagaga ccacccaatt gctggctaaa
gccatcacca aagccccaca accccccaag gcctgggtct
tagtcacagg tgtagcttac taccagccca gtctgactgc
ggagtatgat gaagacagcc caggagggga ctttgacttt
ttctccaacc tcgtaaccaa atgggaagct gcagccaggc
ttcctggaga ttctacacgc caggtggtgg tgcgctcagg
ggttgtgctg ggccgtgggg gtggtgccat gggccacatg
ctgctgccct ttcgcctggg cctggggggc cccatcggct
caggccacca attcttcccc tggatacaca tcggggacct
ggcaggaatc ctgacccatg cccttgaagc aaaccacgtg
cacggggtcc tgaatggagt ggctccatcc tccgccacta
atgctgagtt tgcccagacc ttgggtgctg ccctgggccg
ccgagccttc atccctctcc ccagcgctgt ggtgcaagct
gtctttgggc gacagcgtgc catcatgctg ctggagggcc
agaaggtgat cccacagcga acactggcca ctggctacca
gtattccttc ccagagctag gggctgcctt aaaggaaatt
gtagcctaag taggtcgtgg caagggcctg aggcctgttc
ctcacaggct tccaggttag gcactgtgaa taggctcagc
tcctctagag agctgaagcc atctggttct tagattcctc
tcccagtcct ctttcccatt gttctgttgc tccaccttat
tgtctcaagg ccgtaatctc atcaggttgg gacattaatc
ttttcaactc cttgtaagat ttcccagttt ggtttctcta
catgtcctgc agctgcccca cttctccttt acgctgtgta
gagaatgctc tgcagtttag gcaataaaaa taaattgtct
cactaa

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of Homo sapiens regulating synaptic membrane exocytosis 2 (RIMS2), transcript variant 1, mRNA having NCBI Reference Sequence:

NM_001100117.3 (SEQ ID NO: 61):
agttcccctt tcccttgaac cgctcacttc acagcccttc
gcccccggga agaagaaaca tttcccgaag cgcactcctc
agccctcctt ccccacgcgc tcgccctccc ctccccctgc
ttttcttggg ggaggggggc tgtcgccttg gattgaaggc
cattgatttg tatgtatttg tcccagcgct ggaggctgcc
ccagccgccg cgccggtgcc gccgctgcca gtggagttgc
ctccccgctt ccctagggtg gttcggctcc accaaacatg
tcggctcctg tcgggccccg gggccgcctg gctcccatcc
cggcggcctc tcagccgcct ctgcagcccg agatgcctga
cctcagccac ctcacggagg aggagaggaa aatcatcctg
gccgtcatgg ataggcagaa gaaagaagag gagaaggagc
agtccgtgct caaaaaactg catcagcagt ttgaaatgta
taaagagcag gtaaagaaga tgggagaaga atcacagcaa
cagcaagaac agaagggtga tgcgccaacc tgtggtatct
gccacaaaac aaagtttgct gatggatgtg gccataactg
ttcatattgc caaacaaagt tctgtgctcg ttgtggaggt
cgagtgtcat tacgctcaaa caaggttatg tgggtatgta
atttgtgccg aaaacaacaa gaaatcctca ctaaatcagg
agcatggttt tataatagtg gatctaatac accacagcaa
cctgatcaaa aggttcttcg agggctaaga aatgaggagg
cacctcagga gaagaaacca aaactacatg agcagaccca
gttccaagga ccctcaggtg acttatctgt acctgcagtg
gagaaaagtc gatctcatgg gctcacaaga cagcattcta
ttaaaaatgg gtcaggcgtg aagcatcaca ttgccagtga
catagcttca gacaggaaaa gaagcccatc tgtgtccaga
gatcagaata gaagatacga ccaaagggaa gaaagagagg
aatattcaca gtatgctact tcggataccg caatgcctag
atctccatca gattatgctg ataggcgatc tcaacatgaa
cctcagtttt atgaagactc tgatcattta agttataggg
actccaacag gagaagtcat aggcattcca aagaatatat
tgtagatgat gaggatgtgg aaagcagaga tgaatacgaa
aggcaaagga gagaggaaga gtaccagtca cgctaccgaa
gtgatccgaa tttggcccgt tatccagtaa agccacaacc
ctatgaagaa caaatgcgga tccatgctga agtgtcccga
gcacggcatg agagaaggca tagtgatgtt tctttggcaa
atgctgatct ggaagattcc aggatttcta tgctaaggat
ggatcgacca tcaaggcaaa gatctatatc agaacgtaga
gctgccatgg aaaatcagcg atcttattca atggaaagaa
ctcgagaggc tcagggacca agttcttatg cacaaaggac
cacaaaccat agtcctccta cccccaggag gagtccacta
cccatagata gaccagactt gaggcgtact gactcactac
ggaaacagca ccacttagat cctagctctg ctgtaagaaa
aacaaaacgg gaaaaaatgg aaacaatgtt aaggaatgat
tctctcagtt cagaccagtc agagtcagtg agacctccac
caccaaagcc tcataaatca aagaaaggcg gtaaaatgcg
ccagatttcg ttgagcagtt cagaggagga attggcttcc
acgcctgaat atacaagttg tgatgatgtt gagattgaaa
gtgagagtgt aagtgaaaaa ggagacatgg attacaactg
gttggatcat acgtcttggc atagcagtga ggcatcccca
atgtctttgc accctgtaac ctggcaacca tctaaagatg
gagatcgttt aattggtcgc attttattaa ataagcgtct
aaaagatgga agtgtacctc gagattcagg agcaatgctt
ggcttgaagg ttgtaggagg aaagatgact gaatcaggtc
ggctttgtgc atttattact aaagtaaaaa aaggaagttt
agctgatact gtaggacatc ttagaccagg tgatgaagta
ttagaatgga atggaagact actgcaagga gccacatttg
aggaagtgta caacatcatt ctagaatcca aacctgaacc
acaagtagaa cttgtagttt caaggcctat tggagatata
ccgcgaatac ctgatagcac acatgcacaa ctggagtcca
gttctagctc ctttgaatct caaaaaatgg atcgtccttc
tatttctgtt acctctccca tgagtcctgg aatgttgagg
gatgtcccac agttcttatc aggacaactt tcaagccaaa
gccttagtag aagaacaacg ccttttgttc ctagggttca
gataaaacta tggtttgaca aggttggtca ccaattaata
gttacaattt tgggagcaaa agatctccct tccagggaag
atgggaggcc aaggaatcct tatgttaaaa tttactttct
tccagacaga agtgataaaa acaagagaag aactaaaaca
gtaaagaaaa cattggaacc caaatggaac caaacattca
tttattctcc agtccaccga agagaatttc gggaacgaat
gctagagatt accctttggg atcaagctcg tgttcgagag
gaagaaagtg aattcttagg cgagatttta attgaattag
aaacagcatt attagatgat gagccacatt ggtacaaact
tcagacgcat gatgtctctt cattgccact tccccaccct
tctccatata tgccacgaag acagctccat ggagagagcc
caacacggag gttgcaaagg tcaaagagaa taagtgatag
tgaagtctct gactatgact gtgatgatgg aattggtgta
gtatcagatt atcgacatga tggtcgagat cttcaaagct
caacattatc agtgccagaa caagtaatgt catcaaacca
ctgttcacca tcagggtctc ctcatcgagt agatgttata
ggaaggacta gatcatggtc acccagtgtc cctcctccac
aaagtcggaa tgtggaacag gggcttcgag ggacccgcac
tatgaccgga cattataata caattagccg aatggacaga
catcgtgtca tggatgacca ttattctcca gatagagaca
gggattgtga agcagcagat agacagccat atcacagatc
cagatcaaca gaacaacggc ctctccttga gcggaccacc
acccgctcca gatccactga acgtcctgat acaaacctca
tgaggtcgat gccttcatta atgactggaa gatctgcccc
tccttcacct gccttatcga ggtctcatcc tcgtactggg
tctgtccaga caagcccatc aagtactcca gtcgcaggac
gaaggggccg acagcttcca cagcttccac caaagggaac
gttggataga aaagcaggag gtaaaaaact aaggagcact
gtccaaagaa gtacagaaac aggcctggcc gtggaaatga
ggaactggat gactcgacag gcaagccgag agtctacaga
tggtagcatg aacagctaca gctcagaagg aaatctgatt
ttccctggtg ttcgcttggc ctctgatagc cagttcagtg
atttcctgga tggccttggc cctgctcagc tagtgggacg
ccagactctg gcaacacctg caatgggtga cattcaggta
ggaatgatgg acaaaaaggg acagctggag gtagaaatca
tccgggcccg tggccttgtt gtaaaaccag gttccaagac
actgccagca ccgtatgtaa aagtgtatct attagataac
ggagtctgca tagccaaaaa gaaaacaaaa gtggcaagaa
aaacgctgga acccctttac cagcagctat tatctttcga
agagagtcca caaggaaaag ttttacagat catcgtctgg
ggagattatg gccgcatgga tcacaaatct tttatgggag
tggcccagat acttttagat gaactagagc tatccaatat
ggtgatcgga tggttcaaac ttttcccacc ttcctcccta
gtagatccaa ccttggcccc tctgacaaga agagcttccc
aatcatctct ggaaagttca actggacctt cttactctcg
ttcatagcag ctgtaaaaaa attgttgtca cagcaaccag
cgttacaaaa aaaaaaaaaa aaatcacagg ttgcaaaccc
tggtaacact gcatgcttaa tgttgtgtct tctgagcctg
tttctaggga tacaaagcaa tcctgtgttc tcagaggaag
ttgcacacat tgtgccctaa agaaggccct caggtgaaag
agcagagctg tgaagaacta tcagatttgg aattcaatga
cactcgagtt ctggtccaat ctgaagccat ggattaatct
caaagaatca gtcagtttca tgcaacagaa gcccttttca
atggcacctt tatattttta tcattccttt ttcttcattt
atctaacccc aaagccctga tatgccacag aaatggagct
atacagccat gaagcggtgt tacaggtgag gagtgtaatc
ctaggaagca tcaggtgaaa agcaggagac caaagaagtg
gtcaggaaca atcatcagcc ctcctctggg cgggaatcag
agcagtcagt ccagcaggaa gagtggcaga ctttgtagct
ccatgggcac gtcaattact aatgctaaga tgtgttggac
tctgaaaaac aaaattctgt ggctacactg tactgaatga
aattaaagaa actttttttg catggacaca gattagctga
atacttaaat tattttcttg gggctgcaac ttgcaaaaaa
aaaaaaaaga ataaaaatca gccattttca acaatttata
ttatttttaa aaataaattt cactagtgca tggttttaaa
aaggagagag aatgcaacag ggtgatacaa agatacacca
tgtttattct ttaatcatag tctgtgtttt ggcagacatt
acaaatggaa atactttcta gaagatactt aaaattctct
ttatgtgaca aataagtata atatattcaa tttatttcca
tgttaaatat acaaatctta tgaagttcaa tatgtgcaaa
tttttcacat ctttctcctt ctctcacttt acctcttctc
cctcttttaa acttttcttt ctccctgcca gagtgaacct
tatactaaaa aattacaagt tttgatctga tcctctctca
taccccatgt ttgattcaga gctgtagatg cctctgaatt
tgcgaatttc tcaagggaaa attaacttta agagctttct
ttatttcaag catgttgaaa aggattttgc aacatgactt
gggagtacat taaagtaagt cagcatgtat ttgacgaaga
agatatttga acttttgcag tttattgtac agtgcatggt
aattttttca cctttaaaat tcagtttaca ggaaaattct
aaaatcatgt tgccattgtg atgtccaata aatttgtttt
tagcaccagc attattcata caggggttaa agtattattt
gtagaaggtc ttaggttttg tttgtttttt aatcatttaa
agcaatttct ttagccagtt tccatttact atgtgaatag
aagcactgct aaaaattggg aaccctgaaa cacagggctg
tttattaatt catttttctg tagtaaaatt caatttttca
caaattatat ttctaaagaa atatagtaaa cataaatttg
caacaatttt aaagctccag tttttaggtg actcaaagaa
agtcattatg cctattaata gttatttgat gccatcacca
aaagtctatg tgaaaatctc ctaaagtcaa aacccctgcc
tttggtttta cagacggtta ttaccattgg gtggagctgc
aaggtcaaat ttctcctaag ttcccctatt tagaggaaaa
gtcactggtt attgtaataa accacccatg gttctttatg
tacattttga taacacatta ttatagcttg attttaattt
tttgcattaa tttttgaaat ccacatacat ctcatttgtt
taaattaagg ccatgcacaa atattttttt tagttcagtg
ctgaccatta aaaactatca tgcttgatac ggtgcaaaag
ttaaaatgag tatcactaaa aatgccttct ttttatgtgg
tgcaatatga aatacaccaa gactgtgtct tgacattctg
atggacccag gtaaagttgt taaaagaacg aataaaactt
tattaaaata atttagacac ctgtgtacca gcaacaattg
atttaataga cctatagtgt ctatactatc ccttagaata
aaggtttatg attttcctga tactaagatg cagtcacata
atcttttgtg catattccta tacaaattat ttctaatttt
aataagaagg acgtgactac ggaatatttg tacatacttg
tcattatgca gtatttattt aaaagttggt gttttttttt
aattttcaca tctgcacctc gacttgtggt ttagtcatgt
aactagcact atgccagtga ccgttgttgc cctgtacata
gtatgtttga aaagtaaagg gaattccagt tgggaaaaaa
gggcagatta gtcctgtaat gaacaccaac taatgtaaat
caaattcatt ctggtgatgg tatttaacac tttaaataaa
acattttctt tacaggcgtc tgcagtgctt tctctgactt
ttctccccac acagccctga gcctgctgca gctcattccc
tgaactcatg tgtcatttaa agaatgaaat caccgtctcc
tacttctcga taacataagt ggactgctgg tcttagcagc
ggccctcagt agagcatttc tttaaaacgc caaaggattt
ctgctcacac tatgaaaagg tgctgttttt taaaaggttg
ttattttgga ttgagtttct ttctgattaa atgactcagc
aactcacaga ttttttgagt gaaattttta atttagtcat
ggccttcact gacagcatag tcacaaatac tcaggcacag
gctctgctag cccctgggtg aagatggcga aggcataact
ggctttatgc agcatatgtg tttctgctaa agtgtcagtt
ttgctttgtg gggagtggag ggtgtgtttt cgggatgggg
agacgtggta acctgacatg taacaacctg tccggagact
agcttctacg tgtggatatg aatgggtgag aggatttctc
catatccttc tggggcgatt cctcaactgg gagaaggaaa
ccctgcagag ttctcatggg agtctgcttc aggtttgaaa
tttaagagct agtttggatt catgtttagt aatcgaactg
aaatctaagt ctagctgtct ctctattctt ggaaacaacc
atttcctcca tttccaaaga ctcaactcga gtccaattcc
ccctatctgt cccatatatt tttcctttat cccatatata
ccccctactc tagtgaattg tttctttgtt gttcattcct
gttctttgtt gttcatatac attcctgtat atgaacaaca
ttttccttta tcccatatat accccctact ctagtgaatt
gtttctttgt tgttcattcc tgtttctttg ttgttcatat
acattcctgt tataaaaatt cccttccctt tcttatgtgc
cctctcctga aaagcccttc tacttttctc aataatgatc
catgcgagtc ccttcttgca actcccagct cacgaatgag
ctctttcggc aactcctgac taaaccctaa caacatggct
gccattgatg ccaacacctt cactttccca gggaccccag
atgccaaggc tccataggca acaataaagg atatgatggt
cctgtagtgg gtatgataga attaggcaag agatcaccaa
agctgtctgc ctactactga tgtaaacctt gacattctgt
gcacgtaaaa atcatgtgct caatgtgttt gtctcaactc
cctcagctcg tgatgccctc agggttctgt gggcatttat
gcactgaaga aacaggagtt cacacatcca cctctggact
gtgaaatgtg tattgagaaa tactttgcaa gagagaattt
ttttaagtga acaaacaaca agtctgtgcc acacacatct
tccatatgcc ctgactcagg tcacttaatc tccaggattt
catttcctca cctggaaaat atggagtttg aggtagattc
tcatctatca ttaaatcaac actttaacta aaacgtaagc
tccttcaggg cagagaccgt atcttcagta tcaaaaacaa
tgtttgacac atagctgctc aataaacatc tgtccaatga
a

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of Homo sapiens ST8 alpha-N-acetyl-neuraminide alpha-2,8-sialyltransferase 3 (ST8SIA3), mRNA having NCBI Reference Sequence:

NM_015879.3 (SEQ ID NO: 62):
ccttcgccac gccgccgcgc agcccctcca tcttcctgct
cggcaccggg ccccgcgcgc ccctgcctac ggggtcccgc
tgctctccgg ggctcctgcc agccccaacc cccggccccg
gtggcctccc cccacccccg cccgggtccc cctcctccgc
cacacgcgcg cgcgctcaca cacacacaca cacacacaca
cacacacaca cacatatata cacgccagcg agctgctggc
cgctcaatgg accgatttcc ccggtttccc tgaacccagc
ccagcccggg atgagaaact gcaaaatggc ccgggtcgcc
agtgtgctgg ggctggtcat gctcagcgtc gccctgctga
ttttatcgct catcagctac gtgtccctga aaaaggagaa
catcttcacc actcccaagt acgccagccc gggggcgccc
cgaatgtaca tgttccacgc gggattccgg tcacaatttg
cgctgaagtt tctagacccg tcattcgtgc ccattacgaa
ttctctcacc caggaactcc aagagaaacc ttctaagtgg
aaatttaatc ggacagcgtt tttacatcaa aggcaagaaa
ttcttcagca tgtcgatgta ataaaaaatt tttctttgac
caagaatagt gttcggattg gacaactgat gcactatgat
tattccagcc ataaatatgt tttctctatt agcaataact
tccggtcact tcttccagat gtgtcaccca ttatgaacaa
gcattataat atttgtgctg tggttggaaa tagtgggatc
ctgacaggga gccagtgtgg acaagaaata gataaatcag
attttgtttt ccgttgcaat ttcgccccta cggaggcttt
ccaaagagat gttggaagaa aaaccaatct taccaccttc
aaccccagca tcctggaaaa atattacaac aatctcttga
ctattcagga ccgtaacaac tttttcctca gtttaaaaaa
gcttgacggg gccattcttt ggatccctgc atttttcttc
cacacttcag caactgtgac caggacatta gttgactttt
ttgttgaaca cagaggtcag ttaaaagtcc aactggcttg
gccgggaaat ataatgcaac atgtcaacag gtactggaaa
aacaaacatt tgtcacctaa acggctgagc acaggtattc
ttatgtacac ccttgcatca gcaatatgtg aagagatcca
cttgtatgga ttttggccgt ttggatttga ccccaacaca
agggaagatc ttccatacca ttactatgac aaaaaaggaa
ccaaatttac caccaagtgg caggagtccc accagctgcc
tgctgagttt cagctgctgt accgaatgca tggggaaggg
ctcaccaagc tgactctgtc acactgtgcc taagaactcc
aaacggaaag cgccaaatgg ctgtttaaaa agtgccccaa
atcaaattga atagccttca gaatagaacc ctagagaatg
tcttataagg attgtctgcc atttaaaagg aaagatgtct
tttctctttt gcactgctct tttaagagtt ttagcagatt
tagcaggaca gatgcattga agccacatgg tttagacttg
attgataaag ggaatgttgc atttgggact atgctgctaa
cgaaatggtt tgaagtattt tcatgtttgg attttaataa
taaactgcct ctcattttta tgaggactag agctatagtt
tctgcagctc tgctcagata gtcctcataa tcagaggcct
ctggccaact ggggcaggac ctgttttgct ggtgggatca
gactctgaaa aatggaaacg taaaaaactg gtttgcatat
ctcatcttct atctatctcc ctatctccat ctctatcacc
atctcactcc ttccctctgc aactactccc tgccctacca
ccgtggagtt taataatttg ctaggaatcc tattgaattc
gctttgcttg tatatgttgc atttgtactt gatgtgttta
aggttctggg taactttgat gcttcatgac aaaataggca
aatcacgatg gactcccttg tgattccttt tttaaagaaa
tgcctctgat gcagtcctca ccagtccatt cagtcattct
acagcaagat ggtccctctg tggtgagtgg gatataaggc
agtgcatctt tcatgatcac aaagaaagcc tatgttgtgg
atagcattgc gtctcttgat gtaggcattg ttatggcaaa
taatagcact gtggccacat ctgtaaggcc attccagcct
aaatttagtg tctcccataa tcccaaccca tgcactaggc
tgggtgttct ctcagaatat agaggcaatg aatcagctta
agaccctggg acacacaaag gaaaaggaag aagaatattt
agaagttgtt atggaatcct gtgtacccta aagtgtggta
accatgttac ccattccagc tgggttaaag tggaggagga
tcttggccag gccaggtgcc caggtgacta gtgctgaggt
gcgatcgttt tccgtatcat tgtgtgcacc accaccaatc
cagaaccatt tgctctcttt gagaacattg tagcactgga
tattccctct ttccactgaa gtcagatcta tacaactaca
aattaagcat ttcaaaagaa aaagccattg tgcagtggca
agagttggaa tccaagatta tgtagacctg cgatgaagga
agtagctggc cattctcatt ttctttattt cagccatatg
agtagaaatc aaatcattcc catccctggc tttttactga
gcagcacttt gttcttccaa ttcagcccaa cgtctggcca
agttgatgtt aaattttaat aagagtttct ttcttctttc
caaatgccag tcaagcatat ttgttatgca tttgagtggg
gtagctcatt cttttcctga aatctcatct agattgcttg
caatgcgaca aataactgtt taaaaaatgt gctgtagttc
agtgatgagc tcattgcccc tctttaggaa ggtaagatca
tgcctaaaat taaaccgaat atggtaagga aagacactat
cgttatgctc acgttctgcc tggtcctaaa cctgccttat
tattattttc agaaagcaat ctaattagag caacttgcat
agatctagat attaactaat gaaagaaaca ccaagttcaa
atgtgttacc aattatgaca cacattacaa tataattata
aggggagaca ttaaaaacat tttgtaattc agtggtataa
tttgatgggt atcacaacca caccgtgttt gttccctgtc
cctttgaaga ggactgtact accatcctca cactgtgctc
tgttgggatc actgagagat tgacaactga cagaagtgtt
tagttggctg ttcctgagca cttagttcat ttggttatag
ctaagtgata ttaatgccaa tattacttgg gccagattct
gactctggag ttttaatccc aatgtgccca ttcattttca
ctaaatataa atgcatagac actttaaacc aagaagatta
aaagaggaaa agaccagaat ctctaaaaaa cagtttactg
ttctgtagtg gacaatttaa attttatctc atcataatgc
caaggatttt tgtttgtttt ttaagttcag ggagattttt
taaatctcct tgcagtcttg gctccatctt aaccttagag
tgggaggact ttcccatccc ccaccaccgt gttatctatc
actttaatga aaactcaaaa tagtgaggaa atggactttt
cttcagaact tgaggggttt agagatcagg ctcatctctg
cttataagga ggacttcctg gagatgatcc caatagatca
cccttcactt ttgaagaata gcagcttcta gctgaccatg
caaaatctct gtttgttttt aaagactgtt tcaaagatta
aatctataca accactttta tgaccaaaaa agttgttgtg
tcccaagtgt cctaattaga aaggcagctg gctctcctgc
agctcccagg cttgtgtagt caagtgcagt gaactgggca
gtaatcagct caggaccatt ggctctttcc tcctctttca
gctacagaca catgccctgg cttttgcatt gaccttgctt
tgttagttct agactaattt atccttttgt cagacacttt
ccctttttta ggatactcac aacagagtcc actttcagct
attggaatac attttaccat aaagccccca aggctttcta
cccataacac caacacaaaa aaccaattaa gttgcagagg
ggggaaataa tcacaaactt ttggcaacct gcttaatgct
agacgctctg ttttcactgg ttattctgag tgtcatatgc
agatgcagat ccaagggaga cagggctaaa tggtggtgtc
aagaattcct cccacacccc aaaaattctt tccaatattt
gattattaaa atatctatca tttctccacc ttgtgtctgt
gtcttaagtg tctgtgaata ttgtaaaagt gctgtatgtt
tagtagtgtt gtgtgcctgg cagtgctgac tatgactact
gtgccatctg tctgtgacct tgatgtcagg tacctggcca
tggggctacc agcaaggatg tgcaaaggaa gaaccgctgc
ccctgccctc agcttcctta tgcccgagcc actacttatc
cgtgaatgtg agtgccaaga gaaacctaat ttggtgggga
agccaaggaa tgggagaacg ttttttctga tttgagtcaa
ggcactaatt attagacact ctcagacaac aaagcggtat
tgacctgaga tcaaaggaag cagggacaat attgtagaat
gctaggacac tggaaatgaa gaggccttca gcttcgccag
tcttgattct tgactttaca aatgagaaaa tggagaaaca
gggaggctag gtgatttgcc ccagatgaca ctgtcaatta
gtggagaaat tatgattcct cctctagtac ttcttggtct
taccagcatc aaatgaagct gggctcacag ggaaagtggt
ttttggtctg ggatctgcag agcacataaa ataagttaga
aagtctataa aagaggaaaa aaggttctcc tctccctccc
aaaaatgact tcccaacaca gaacaataaa ggagatgctt
gtgtgggtat gcaactgtcc accaattatc acctaccttc
tccctgagat gaaacaaaac acgcttggta aatcatgatt
tgataaataa caattaagtt actggtggtt catgcttgac
agctgcaatc ttacccaata agctattcat cttagtaaaa
ccttgtttta gtaatataca tgcataattt agaagttgaa
ggctgaaaat tgttgtgttc ttctccatat atatacaact
ttcatcgggg tcccttgagt taagaaatgc cttctatttt
ataggtaatc tcaacagcag actttgaaat attgtgtttg
gttcaatatg ctaagaagat atacaaataa taataacttc
gtgagtcttc atgtgcaaaa tgacaaactg agcaattttc
tgtataattc tagattctta aaaccttttt tcccataatt
gtttgaatgc actaggcaca agtttctgtt taagcctctg
cttctccctg aatgtgtaga actgtcactt tgcttacaaa
ggggctgtgt gtttgtggct ggcagcccag atagagcact
ctgggtgctg gggattgagg gcaccttggc cagctgattg
acctgtctgg actctgtggc ccaattaatc gaggccacag
atcagttggt actaagcatc aaaatgattc attatgcctc
tctcacactc cgattgcttt gccacataga taacagcatt
tgaaatggga aacactttag aatgtcatta ttgtattata
tattgtcatc ttatgcagta tttctgaaaa tttattttta
ttgtgatgac tgattgaaga catatgtgta tgaatagatg
ttcctcagtg aagcctattc tcttgttctt gatgacagtg
gcatcagagt gaagagttgt tgcagagtaa ctgacttcag
tttaaggtta ccctggggtt ctggggttag gacccggatt
atgcagctga tctcccagca aacacagctg tatgaaataa
gcaactcttc aaaatacaat tcttgccact cattctggac
ctttgattca taatgtcctg ggataagtcc aacttacaaa
gattcccagg tgattcgtgt gaccaccatt tggaaaccag
taatatatac atccggagtc atatatacca ttttgaacaa
agtagctaaa tgcttatctc tccttgtgcg attagtttca
catgttcgtt gaggttatca tgttaaatgg caatatagac
acagtcttag tgattcaaca attcagctgt ttcagccgaa
ttgaccagtt gctaatttat aagcaaattg taactgatca
atttagtcag aatggatgca agcccaaaga caaggtggag
agcaagcttt aatatacact ttgtctcaag gccagctctt
catatttccc tctttatatt tcaaactaag aggcagatct
caggtcactc atctttagaa ttggtgagaa tgagaaacag
ggaactgaag tgattcattc agattcaatt tggaagtcat
tcccagagcc taaaatattt atctaagggg tctgatttat
caacctcagg tagtctccaa aacacaaagg tattcaggct
aaaataaaac caatcaaaaa aggaaattca ggaggctcca
gttctaagac taacccagta taacaagggt gatcagttat
tacttgtggc cttgtaccac ctctgttgga taatgtgcca
ccgtcctaat gaagctttgg ccagacttct ttctatagct
gtgcacatag aaaggcttta gacaacagtg ataccaaagg
gttcctttga ttttccttct gtgtgattct cagacagttt
tagctctttg gtgtgctgcc aaatgtttaa caactggctc
tggaaaacag tggaacccag atttttagca cctaccaatt
tctatggtgt aaatactctc atctcagcca atttcaggct
actaacttaa tgtcaaccag ctgaaaacac aacaatatgt
tttcaagagc cagtataagt tggctccaat acagcatgag
ccagctccag catagcactg cctgaacaat ttttaagtca
gtgagtacca aataggagag gctagcagag gctttggagg
gacttttaaa tgtcccctcc accaagggaa gcatttccat
gttggttagt tttactgatt tttagtaaaa gagcatatta
ttggatccca aggtgttcaa atattccacg tgactcttag
gaacctctca acagactccc cagtatcttg ggagattacc
aactgtggct tttctaattt ctaagggtga ccctccagga
aaacattctt taatttaact accagttaaa accatgtttg
gctgatcttt tttaaaaaat ctatacctat agttcatcac
ttgtttcccc aactttgtca ttttccctat ccacttgccc
ctcatccttc tgacacaata aaaaaaatac aggtacccac
aaaacagtgc ctgcaaagga gcaggggaag aacaaggcac
ccagtaagct cacttggctc tacatatctg gaaaggcact
gaggttccaa ggtctctacc ccaccagggg acacccagta
aacatcacgc tgagagttta agttgcactc aactggagtt
gatttgccct tagaaaagtt tccaacggtg aagaaatacc
acttggtagg aatatctctg ggcaacttca ttgctgactg
ggcaaacttt gtaggataag gtgtcatgtg atgtaaggct
ggaccatgaa aaacaaaaat ctactaatac acatttggcc
cctgcagtcc cctggcattg agcaatagag caactgtcct
ttctcaccac tagttgtgag tgtacttcca actgagtgtt
tagaaatcag gtaatctggt aatccacaga acaagctttg
tttgcaaatt gcaaattttt ctggtagaag tcattcttag
gtgggcttca ttaaatcttt ggagaggcaa tgctgggaaa
aaatatgcct aattcctagg acacagtgag tggccgtaag
ttgttatctg catgtactaa atcaaaatta gatcatgaag
gaccaaatct acttccgttg ctataataaa ataccctaca
ggttctgtaa ggcatttcta aactgtaaat gcttgaaacg
caagttggac attttctata ctatgtgtaa aatgccaggt
tatacctcta ttttctttct tcctaatggt cattgaaatg
agcttgtttc tctacattga gcaagctaca ttttatttta
aatgagtcag gtgcatgttc agatgtctgt attcccaata
cattgcatgc agcctgagca caagtatgcc ttcaccctct
ggctcttttc ctgacaccaa acagagaagt ccacagctac
aagcacaagg ggcttgacag gtaggccttg ttttattatg
aacaaattca ccagaaaacc attcttgagg aaccagccac
tcccatagca tttttagtct tagggaagaa aattggcagg
gaagaaaaat tggcagacaa tcggaaagag gtttggtcaa
cctgcataag tggaataaag tcattgaagt acttgcaaaa
agaagaggga ggtttccctc tgaaggaatc tagaccattt
tttaatatgg agaataactg taccccatgt tagaagtgct
ctttagccat atttcaagat ccagatctga acccatactg
tgcagaaaag cacaagagcg cttacagcca agagccctgg
gaggagttga gtaacaggct gagatccggt caccaagcat
cattagttag cactgaaatt ctcccaggac acaaaacaaa
tgtatggaaa catcccaata cactatcatt tactcacatg
ggacaaccac ctaagtccat agcaaaactt tacagatcta
gatcttatct caagtcagga ttccagatca acatcctcca
tgtggagatg agacaactga ggttcagaaa gttggagtga
cttatttgcc cagctagcta actggaatta gagtctgggt
ctcccaacca ctgattctgc tacaccacac tgtgctgctt
ccattagtta gatggacatt gacaattaat tgtgaaagca
ttaattagaa gtcaagacaa aagaatgttt gtgatatctt
ctctcgctta acatactttg atttcaccat tgcatttcag
cattttaaag aatagtgtat cagtatagaa ggaggagcaa
agctctttaa gagtaatgat gaaaccaaaa ggtcaaaaag
tagaagaata gcgaatgtaa gttattaaat aaatgagcaa
gaagtatgtg tcagggaaaa taaatgcttc ttatattgtt
aataccgtga ttccctgccc gctcaccccg tcactcaaca
cttgatgcat ttgttttgga aagtgccatt ttatgctgag
atactggtat tgaaaacttc cctctttccc aactctgtca
gaagttctct tgctctttgg aagaagccga gggccttaac
aaatcagact gacctccctt gtgacaggca gcctcgcctg
cccttggaaa caagctgcca gaatgtgagg gccacagaga
cccaagagaa gccagcactg ctagactgag gaacttgtaa
atatcttctg ctttcttggt gaacaaggac agaggcaatt
gcgtgtatat tgtgactgta gtttgtgaag aaaatgcaac
catttgcttc gacagctcct caaatgtact tgttaagtgt
gaatgtgcct gcctcattgc cttgtgttcc aaacacagta
ctgaatgcgt tgtttttaaa taaaccattt cgttttgctt
tgggaaa

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of Homo sapiens cyclin dependent kinase inhibitor 3 (CDKN3), transcript variant 1, mRNA having NCBI Reference Sequence:

NM_005192.4 (SEQ ID NO: 63):
accggtgagt cgccggcgct gcagagggag gcggcactgg
tctcgacgtg gggcggccag cgatgaagcc gcccagttca
atacaaacaa gtgagtttga ctcatcagat gaagagccta
ttgaagatga acagactcca attcatatat catggctatc
tttgtcacga gtgaattgtt ctcagtttct cggtttatgt
gctcttccag gttgtaaatt taaagatctt agaagaaatg
tccaaaaaga tacagaagaa ctaaagagct gtggtataca
agacatattt gttttctgca ccagagggga actgtcaaaa
tatagagtcc caaaccttct ggatctctac cagcaatgtg
gaattatcac ccatcatcat ccaatcgcag atggagggac
tcctgacata gccagctgct gtgaaataat ggaagagctt
acaacctgcc ttaaaaatta ccgaaaaacc ttaatacact
gctatggagg acttcggaga tcttgtcttg tagctgcttg
tctcctacta tacctgtctg acacaatatc accagagcaa
gccatagaca gcctgcgaga cctaagagga tccggggcaa
tacagaccat caagcaatac aattatcttc atgagtttcg
ggacaaatta gctgcacatc tatcatcaag agattcacaa
tcaagatctg tatcaagata aaggaattca aatagcatat
atatgaccat gtctgaaatg tcagttctct agcataattt
gtattgaaat gaaaccacca gtgttatcaa cttgaatgta
aatgtacatq tgcagatatt cctaaagttt tattgacaaa
a

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of Homo sapiens T cell immunoglobulin and mucin domain containing 4 (TIMD4), transcript variant 1, mRNA having NCBI Reference Sequence:

NM_138379.3 (SEO ID NO: 64):
agactcctgg gtccggtcaa ccgtcaaaat gtccaaagaa
cctctcattc tctggctgat gattgagttt tggtggcttt
acctgacacc agtcacttca gagactgttg tgacggaggt
tttgggtcac cgggtgactt tgccctgtct gtactcatcc
tggtctcaca acagcaacag catgtgctgg gggaaagacc
agtgccccta ctccggttgc aaggaggcgc tcatccgcac
tgatggaatg agggtgacct caagaaagtc agcaaaatat
agacttcagg ggactatccc gagaggtgat gtctccttga
ccatcttaaa ccccagtgaa agtgacagcg gtgtgtactg
ctgccgcata gaagtgcctg gctggttcaa cgatgtaaag
ataaacgtgc gcctgaatct acagagagcc tcaacaacca
cgcacagaac agcaaccacc accacacgca gaacaacaac
aacaagcccc accaccaccc gacaaatgac aacaacccca
gctgcacttc caacaacagt cgtgaccaca cccgatctca
caaccggaac accactccag atgacaacca ttgccgtctt
cacaacagca aacacgtgcc tttcactaac cccaagcacc
cttccggagg aagccacagg tcttctgact cccgagcctt
ctaaggaagg gcccatcctc actgcagaat cagaaactgt
cctccccagt gattcctgga gtagtgttga gtctacttct
gctgacactg tcctgctgac atccaaagag tccaaagttt
gggatctccc atcaacatcc cacgtgtcaa tgtggaaaac
gagtgattct gtgtcttctc ctcagcctgg agcatctgat
acagcagttc ctgagcagaa caaaacaaca aaaacaggac
agatggatgg aatacccatg tcaatgaaga atgaaatgcc
catctcccaa ctactgatga tcatcgcccc ctccttggga
tttgtgctct tcgcattgtt tgtggcgttt ctcctgagag
ggaaactcat ggaaacctat tgttcgcaga aacacacaag
gctagactac attggagata gtaaaaatgt cctcaatgac
gtgcagcatg gaagggaaga cgaagacggc ctttttaccc
tctaacaacg cagtagcatg ttagattgag gatgggggca
tgacactcca gtgtcaaaat aagtcttagt agatttcctt
gtttcataaa aaagactcac ttattccatg gatgtcattg
atccaggctt gctttagttt catgaatgaa gggtacttta
gagaccacaa

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of Homo sapiens SYS1 golgi trafficking protein (SYS1), transcript variant 1, mRNA having NCBI Reference Sequence:

NM_033542.4 (SEQ ID NO: 65):
ctttcctacg cagccgctcc tgccgccgtg gtcgctggag
ctttgcctct ctaggccggc agcgcctctc ctccatggtc
ctgtctgtca gcgctgtttt gggagcccgc cggtgaggcc
gggccacgct cagacacttc gatcgtcgag tctgtcactg
ggcatggcgg gtcagttccg cagctacgtg tgggacccgc
tgctgatcct gtcgcagatc gtcctcatgc agaccgtgta
ttacggctcg ctgggcctgt ggctggcgct ggtggacggg
ctagtgcgaa gcagcccctc gctggaccag atgttcgacg
ccgagatcct gggcttttcc acccctccag gccggctctc
catgatgtcc ttcatcctca acgccctcac ctgtgccctg
ggcttgctgt acttcatccg gcgaggaaag cagtgtctgg
atttcactgt cactgtccat ttctttcacc tcctgggctg
ctggttctac agctcccgtt tcccctcggc gctgacctgg
tggctggtcc aagccgtgtg cattgcactc atggctgtca
tcggggagta cctgtgcatg cggacggagc tcaaggagat
acccctcaac tcagccccta aatccaatgt ctagaatcag
gccctttgga catcctgctg acacttgggc cccttaacac
cttgggctgc tcagaccctc cagatgaggt ccagcccaga
tctgagagga accctggaaa tgtgaagtct ctgttggttt
gggagagata gtgagggcct gtcaaagaag gcaggtagca
gtcagcatga cagctgcaag aatgacctct gtctgttgaa
gccttggtat ctgagaggtc aggaagggga cctctttgag
ggtaataaca gaattggaac catgccactc ttgagccaca
atacctgtca ccagcctgtt gttttaagag agaaaaaaaa
tcaaggatat ctgattggag caaaccactt ctttagtcat
ctgtcttacc cccctgggac agctgttacc tttgcagtgt
tgccgaatca cagcagttac ctttgcagtg ttgccgaatc
acagcagttc tgttggagaa acgcttggtt tccggatcca
gagccacaga aagaaatgta ggtgtgaagt attaggctgc
tgtcagggag aggatggcag atggaggcat caagcacaag
gaaaatgcac aacctgtgcc ctgttataca cacgttcatg
tgcacccaag aacctatgac tttcttccag ttccttctac
caggtcccca tcctgctgcc agctctcaac atagcaggcc
ataggaccca gagaagaatc ccagcgttgc tcaaagtcta
accatcataa agacactgcc tgtcttctag gaatgaccag
gcacccagct cccactggac tccaattttt tttcctgcct
tatttagaat tctttggcgg gaagggtatg atgggttccc
agagacaaga agcccaacct tctggcctgg gctgtgctga
tagtgctgag ggagatagga atttgctgct aagatttttc
tttggggtgg agtttcctct gtgaggggct tgcagctatc
cttcctgtgt atacaaatac agtattttcc atggttctgc
ctgcacttac tttgtaatgc cacggttgag attgagagag
atcagcgcag ccaggcaagg gaactttaaa gaattattag
gccaccttct ccctttcctg gaccccagag tcattcctcc
atttggttaa aatactcagt gcagggaact cttacatcct
gtctccttca cttgcagcgt cccctgctat gcctcaggtg
aaccacataa ttcttgggtt tccgttccta cttgctagtg
atttctgaac atgttcaatg gagcggcaca cagtctagac
ccacttccgc attgaaacct tcactgttcc tctttggttt
cttcagagct ttcccaagag agctgtcagt tttcagctgt
cagtaacaca aatgagttta tggtaacaca aatgagtttt
gctatctctc tgagaagctc atctgacctc ctgactctca
gccctacaga gtagggagtt gatgctgaca ggatgaagat
ttaggaataa atatgcctgg gaagagactg ggaaggttct
agggtgaggc acctcagtaa ctcatggtac cttggccaag
ttggaaggaa gcagtttgtt aatgaggcac agtaatcctg
gctgcagggt ctaggaggta agaccagctg ggatgacctt
ccctgggtta atcaatttcc ctctagacaa cacaaactgc
aggcatgtga ctaactttga aagaacaccc atcatgtggc
tgctgtcacc cttgaccagc cgtggtggtg gttactccat
ctgtggttgg agcgcctctt tgggattcac ttcaaggtct
tgtgcctatt tttctgcata tcttctgtga tgacaaatct
ctgtcccctg agtgttaatt tgatttttag aaatggccaa
aagtcacgtg atccaaactt tttttcagta atatggagac
tgagctgcat ggtagttggg gatcaaaaat atgtgacctt
aatgagattt ttatgatttc taaagtaaca ataaaagcag
tttttagagt tgagttccag agagggcagg gcaatggcag
tgacatgttt gtcattttaa taataaataa catctattga
gtgcttaa

a different transcript variant of the gene, or a sequence with at least 80%, 82%, 85%, 86%, 88%, 90%. 92%, 94%, 95%, 97%, 98% or 99% nucleic acid sequence identity thereto.

In embodiments, the nucleic acid inhibits expression of Homo sapiens ubiquitin D (UBD), mRNA having NCBI Reference Sequence:

NM_006398.4 (SEQ ID NO: 66):
gtctctggtt tctggcccct tgtctgcaga gatggctccc
aatgcttcct gcctctgtgt gcatgtccgt tccgaggaat
gggatttaat gacctttgat gccaacccat atgacagcgt
gaaaaaaatc aaagaacatg tccggtctaa gaccaaggtt
cctgtgcagg accaggttct tttgctgggc tccaagatct
taaagccacg gagaagcctc tcatcttatg gcattgacaa
agagaagacc atccacctta ccctgaaagt ggtgaagccc
agtgatgagg agctgccctt gtttcttgtg gagtcaggtg
atgaggcaaa gaggcacctc ctccaggtgc gaaggtccag
ctcagtggca caagtgaaag caatgatcga gactaagacg
ggtataatcc ctgagaccca gattgtgact tgcaatggaa
agagactgga agatgggaag atgatggcag attacggcat
cagaaagggc aacttactct tcctggcatg ttattgtatt
ggagggtgac caccctgggc atggggtgtt ggcaggggtc
aaaaagctta tttcttttaa tctcttactc aacgaacaca
tcttctgatg atttcccaaa attaatgaga atgagatgag
tagagtaaga tttgggtggg atgggtagga tgaagtatat
tgcccaactc tatgtttctt tgattctaac acaattaatt
aagtgacatg atttttacta atgtattact gagactagta
aataaatttt taaggcaaaa tagagcattc aaagccagct
tggaatttaa ttctgtcttg ataccttgtt atttatgcaa
aaactcctat ctcctttcct ttatgacaag agagtaagtt
ttaggttggg atcc

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of Homo sapiens mediator complex subunit 17 (MED17), mRNA having NCBI Reference Sequence:

NM_004268.5 (SEQ ID NO: 67):
agttttgctc cgaaagactt accgaggagg gagcttgcgg
tgcgttctgg gaaagttgct gggccagctc ctttctttcc
agtctgagcg ttgcgttcgg tttcccgagg gtcttctgag
gcaccgcggc tgcgggcttc tgagttcccg gctctccgca
gggaagcctc ctcttcgtac ctcgtttttt ggctcgtggg
gggtcctccc accgctggcc gacgcagcca gcatgtccgg
ggtgcgcgca gtgcggatca gcatcgaatc ggcctgcgag
aagcaggtcc atgaggtggg cctggatggc accgagacgt
acctgccccc gctgtccatg tcgcagaatc tggcgcgtct
ggcccagcgg atagacttca gccagggttc gggctccgag
gaggaggagg cggcggggac cgagggcgac gcgcaggagt
ggccgggcgc cgggtccagc gcagaccagg acgacgagga
aggagtggta aaatttcagc cttccctttg gccttgggac
tcagtgagga acaatttgag aagtgccctg acagagatgt
gtgttctcta tgatgttctc agtattgtta gggataaaaa
atttatgact cttgatcctg tctctcagga tgcacttcct
ccaaaacaga atcctcagac gttgcaattg atatctaaaa
agaagtcact tgctggagca gcacaaatct tattgaaggg
ggcagaaaga ctgactaaat cagttaccga aaaccaagaa
aacaagctac aaagagactt caattctgag cttttgcgat
tacggcaaca ctggaaactt cgaaaagttg gagataaaat
tctcggagat ctgagctaca gaagtgcagg atctctcttt
cctcatcatg gtacatttga agtaataaag aatacagatc
tcgatctgga taaaaagata cctgaagatt actgtcctct
tgatgtccaa attcctagtg atttagaggg gtctgcatat
atcaaggttt caatacaaaa acaggctcca gatataggtg
acctcggcac agttaacctc ttcaaacgac ctttgcccaa
atccaaacca ggttccccac attggcagac aaaattagaa
gcggcacaga atgttctctt atgtaaagaa atttttgcac
agctctctcg ggaagctgtt caaattaaat cacaagtccc
tcacattgtg gtgaaaaacc agattatctc tcagcccttt
ccgagcttgc agttatctat ttctttgtgc cattcctcaa
atgataagaa atcccaaaaa tttgctactg agaagcaatg
tccggaggac cacctttatg tcctagagca taatttgcat
ctactgatta gagagtttca taaacagacc ttgagttcca
tcatgatgcc tcatccagca agtgcacctt ttggccacaa
gagaatgaga ctttcgggtc ctcaagcttt tgataaaaat
gaaattaatt cattacagtc cagtgaaggg cttctggaaa
aaataattaa acaagcaaag catatttttc taaggagtag
agctgctgca accattgaca gcttagcaag ccgaattgag
gatcctcaga tacaggctca ttggtcaaat atcaatgatg
tttatgaatc tagtgtgaaa gttttaatca catcacaagg
ctatgaacaa atatgcaagt ccattcaact gcaattgaat
attggagttg agcagattcg agttgtacat agagatggaa
gagtaattac actgtcttat caggagcagg agctacagga
ttttcttctg tctcagatgt cacagcacca ggtacatgca
gttcagcaac tcgccaaggt tatgggctgg caagtactga
gcttcagtaa tcatgtggga cttggaccta tagagagcat
tggtaatgca tctgccatca cggtggcctc cccaagtggt
gactatgcta tttcagttcg taatggacct gaaagtggca
gcaagattat ggttcagttt cctcgtaacc aatgtaaaga
ccttccaaaa agtgatgttt tacaagataa caaatggagt
catcttcgtg ggccattcaa agaagttcag tggaataaaa
tggaaggtcg aaattttgtt tataaaatgg agctgcttat
gtctgcactt agcccttgtc tactatgatt ttttccagat
gtttcctaaa gaagtttcca gaaactttga cttgaaatgt
ttgcagatca actataagca caaagaagag ataacttcca
aaagagtgct gtttttaaaa ataataatta ggaaatgttt
atttagcact ttcaaacttt tcactttata aatgacaagt
gctttgaaat gcagaagttt atgtacagtt gtatatacag
tatgacaaga tgtaaaataa tatgtttttc atgcagttta
aaatattact aacttaaggg tttctatgtg ctttttaaaa
tattccttct ttgatgttga catcaaataa agtatgtggt
ttaaaaaaat ctccaaatac ctttttttcc ccccaaatac
tttctaaact tttttttttt gagatggtat ctcactctgt
agcccagtct ggagtgcagt ggtgtgatca tggttcactg
cagtcttgac ctcccaggct taggtgatcc ttctgtctca
gccttccgag tagctgggac cacaggcatg cacaaccacg
cctggctaat ttttgtattt tttataaaga cagggttttt
ccatgttgcc caggctggtt tcgaactcgg ctcaagtgat
ctacctgcct ctgcctccca aagtgctagg attacaggcg
tgagccacca tgcccagcct actctaaatt attgataacc
tcttcctcca gttgtctcct ttaagctttc ctgggtctaa
cctacatagg taatttaaga acatcctcag aaaggacagc
tgaaggcaat aggaggcaga ttatctcttt agggcgtcct
caagtttttt tggtctgttc tcccacttga ttgacctcac
cagttgagac acctagtgta tggctcatgc ccagccttcc
acctgggatt ctccagcctc cacccagcag ccctggattg
ctttctccaa ttaaggcctt tccatcagct ctctgctttt
tcaaagcgaa aaaactaatg gattagtggg ttatcttttc
caaggaacag gtttgcactt cttggaaaaa gtgcctaaag
tgtgcccatt aatatgagga tagatttagg ctcataagcc
ttttggtaac actgaaagta gtatcatata ggcaagctct
ccttataagt aaggctttca atttttaaaa cagacatcct
gctttaacaa tttgtaagat gactgtgcag taataaaagt
cctttgtatt tctccaccgt gttttcatta aagaaaaatg
gagcttgtgg gccacgatag aacaactttg tgcttttttc
cccttctgat caagatcttg catctttcta tccatggaaa
ttaaaataat tggtatgaat ttgcagttat ttaaaaatct
tgagtgcttc aaaaattatt gttgcctgca aaatttgcct
tggtcaatag gctaatctgc acaattccac tcacataagg
agtcttttat gtgattttga aggctcaggc tagaagagtg
agtctgagac ttttgctgaa tgaccagttt ttgtttatat
aaacttctcc cattgcagat tgatactttc gtaaactaat
aaaaatgaat tcctaaaatg aaattttgaa aagatacaaa
ataaaagccc catttatttg attataactt gattaaattg
catcaaatac tagaatttat agacagagtc tcactctgtt
ccccaggctg gagtgcagca gcactgtttt ggctcactgc
aacctctgcc tcctgggttc aaatgactct catgcctcag
cctcccgagt aactgggatt acaggtgtgt gccaccacga
ccgtctaatt tttgtaattt tgataagagg gttttgccat
cttggccagg ctggtcttga actcctggcc tcaattgatc
cgcccacctc ggcctcccaa agtgctagga ttacaggcat
aagccacagt gcccagcccc cccaaatata aacatttctg
aatgctttat tttttatttc tctgcttgtc atgaatcagt
aacaaatcat ggaccaggac cacaccttga gtagaatggc
tgagaataca tgtgcagata ctaccgtctg ttcttttaaa
ccccatctga gtagagtggg ataactgaag actttacgtt
cttcatgtct tactttccct gtttggtacg tcgctgtagt
gagtagccag taccgaccta aagaattgta gaaactaaag
caaatgtgtg ggaaaatggt agcttagttg ctgtggtagc
aattcttatg ccttgtattt atttacattt tctagtttaa
tgttttaacc tgaatttcct ggagtttgaa ggatgtgcta
tggaaacttg ggagacagtt tgaagaaaac caattagccc
ctcaacaagt attaacaggt tggcaaggag ctgtgtttga
atcttggctc tgctactggc ttgctgtatg aacttggcaa
ggattctctg tgaacttgtt tccttatata taaatgaaga
tagaggtgcc tcctctacta acctcagtgg tgattgagaa
gtttcagtaa cgttggtaat aaatgttaaa ttctaaagta
ctacataaat ataaagcatt aagcaagtgt gcttctaaga
gtcaagccaa ttagaaaaaa tggttgagac accagctgta
tttattagga gaaagcattt cagaatgtcc tgtattcata
tttgtatgat gttttatata tggtgaagat attgagtgtt
tttcatcaga tttctttgct ggaacaccat caaatcaaag
ggataacctg attatctcat gttgatcagg aattgtaatt
ggcccttaaa tgctgggatt acaggtatga gccaccatgc
ctggcctcct taggtattgc tgatgaataa aaacaggggc
aactaca

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of Homo sapiens peroxisomal biogenesis factor 13 (PEX13), mRNA having NCBI Reference Sequence:

NM_002618.4 (SEQ ID NO: 68):
agtcaggggt aggagcggga gccgagagga ggcggaggag
atggcgtccc agccgccacc tccccccaaa ccctgggaga
cccgccgaat tccgggagcc ggaccgggac caggaccggg
ccccactttc caatctgctg atttgggtcc tactttaatg
acaagacctg gacaaccagc acttaccaga gtgcccccac
ctattcttcc aaggccatca cagcagacag gaagtagcag
tgtgaacact tttagacctg cttacagttc attttcttct
ggatatggtg cctatggaaa ttcattttat ggaggctata
gtccttatag ttatggatat aatgggctgg gctacaaccg
cctccgtgta gatgatcttc cacccagtag atttgttcag
caagctgaag aaagcagcag gggtgcattt cagtccattg
aaagtattgt gcatgcattt gcctctgtca gtatgatgat
ggatgctacc ttttcagctg tctataacag tttcagggct
gtattggatg tagcaaatca cttttcccga ttgaaaatac
actttacaaa agtgttttca gcttttgcat tggttaggac
tatacggtat ctttacagac ggctacagcg gatgttaggt
ttaagaagag gctctgagaa tgaagacctc tgggcagaga
gtgaaggaac tgtggcatgc cttggtgctg aggaccgagc
agctacctca gcaaaatctt ggccaatatt cttgttcttt
gctgttatcc ttggtggtcc ttacctcatt tggaaactat
tgtctactca cagtgatgaa gtaacagaca gcatcaactg
ggcaagtggt gaggatgacc atgtagttgc cagagcagaa
tatgattttg ctgccgtatc tgaagaagaa atttctttcc
gggctggtga tatgctgaac ttagctctca aagaacaaca
acccaaagtg cgtggttggc ttctggctag ccttgatggc
caaacaacag gacttatacc tgcgaattat gtcaaaattc
ttggcaaaag aaaaggtagg aaaacggtgg aatcaagtaa
agtttccaag cagcaacaat cttttaccaa cccaacacta
actaaaggag ccacggttgc tgattctttg gatgaacagg
aagctgcctt tgaatctgtt tttgttgaaa ctaataaggt
tccagttgca cctgattcca ttgggaaaga tggagaaaag
caagatcttt gatatctttc atgtttgcct gcagttgaac
aatactttag agtacttttt aaaattattt ctcacaaaga
aatgaatgta caatccaatg aaaacatttg ttattggcta
tttcaggtgt tttgctgcta gaaattatta aagttacaca
ctagtatgtt ggtctggtga cctggttaca ttttattata
cacattattg gaccataagg acatttgttt cacctagatt
ttaagattat ggagactgct gtcattttta tcttatttaa
atctctaggt ttattggaag agtaagattg atgaactata
gcatgcacag tttggtacag tagagatcat taatactttt
aaaagttctg cattaattga cttggaatcc ttagaaatgg
agtggtgaca tgtcagtatg agaacaggca aaaggtaaat
tttttttttt ttttacaacc ttaagtaatc tcaataataa
aattttctga tctgtattat atccagtgtt gggtttatat
tttcacccac aaacaactga cactgctatc ttttactgta
tttttaaaaa tttaatttga aaaggtcacc cagaagcatt
ctgaaggaaa tggttctaga attatagagt atgtagccct
aatagttttc cctcctagca aaaagtctga aatttatctt
tcttacaaac tgttccattt cttctaagga tcccttaatt
atttatctct ttaagccagg catggtggct cacgcctgta
atcccagcac tttgggaggc tgaggcgggc agatcacctg
aggttgggag ttcaagacca ccctgaccaa catggagaaa
ccccatcttt acgaaaaata casaattagc taggtgtggt
ggcacatgcc tgtaatccca gctactcggg aggctgaggc
aggagaattg cttgaacccc aggaggcgga ggttacgctg
agccggagat ctcgccattg cacttcagcc tgggcaacaa
aagcgaaact ccatctcaaa aaataaaatt atttatttct
ttaaaatatg actagttttc atactgggtg aatcagaagt
atatgaaagg tatactttct tttcctacaa caaatacttg
agttcttttg agtttgcact taatgatata atcagttata
atagtaattt ttaatatttt catagattgt ttgcttctac
cttgtgtaat tttttaaatt ccatatttag gatgctctgt
aaatattgaa aatgtgtcac attggataca tttttctttt
aggtttagtt tttactactg agacttattt atagtcttag
tgctctattg ccatttagaa tatgataatc ctcatgcctt
taatctcagc actttgggag gccaaggcgg gcggatcacc
tgcggtcagg agtttgagac cagcctggcc aacacggtga
aaccctgtct ctactaaaaa tacgaaaatt agctgggtgc
agtggcgtgc acctgtagtc ccagctactc aggaggctga
ggcaggagaa tcacttgaac ccgggaggca gaggttgcag
ggagctgaga tcatgccact gcactccagc ctaggggaca
gagcaagact ctgtctcaaa acaaacaaac aaaaaataat
aatacgataa tgctatttga cgtgtttttt ggtttataat
gattttaaat gcagttaact ttcagtacac tgaatatttc
cccagaaaat tggaaacttc atatacttgg ctacgaacat
actacagagt aatactatca ggaatacagg tgtataagaa
tacatttata gatattgttg aaaactttga actgtttgat
aaaaattgtg atttagtatt ttttcttttg tctttttttg
aaacggagtc tcactctgtc gcccaggctg gagtccagtg
gcgcgatctc ggctcactgc aagctccgcc tcccgggttc
acgccattct cctgcctcag cctcccgagt agctgggact
acaggcgcct gccaccaggc ccggctaatt ttttttgtat
ttttagtaga gacggggttt caccgtgtta cccaggatgg
tctccatctc ctgacctcgt gatgagcccg cttcggtctc
ccaaagtgtt gggattacag gcgtgagcca ccgcacccag
cgatttagta tttttttcta atagactatg ttcaacaaat
aagtaattct cgaatagttc agattaaaac atacaggaac
caagtacata cccagcatag aagaacttta ctaaaggctt
cttggaaagc ccttttttga aacgacagta tcgtaagtaa
catatcattt ataatagaaa tcttgaccca gtgcagaaaa
ataaatatag taaaatttat ttatctttgg ccagttttcc
aacacccagg tattagcctt gaagttgaag agataaggtt
tcttgtatat tatttttcat ttgtgttcta caattaaagg
ttctgctttg atttgtcaga taatttatag aaattttgtt
ctcaaaacaa atgtttgata aaacagatta ttaaatttgg
ggttgagatg tctaaattga atgctagaag taaagtagaa
gtgaccacta ttaaagatgt atagggagaa gagtacagca
caaagtgata aaatagtgac acttttgtag gggtgtttat
tgtttggtta agtctgctaa attacggtat gcattatctg
gtgactattt gtgcctgaaa attcgttttg tattaaaatt
ctggagaagg aattcaggac tagaataaag agaaattttg
taaccttttt tatcatgaca gttttagaat aattttttta
gctgagatta aatgttcaag gctccaatat tatttttagg
aacttattta aggagtgcta ctttacagaa attactaaca
caccaaaaca ttattaatta aataaaatat aagtttacaa
taataaaaca tgtttctttt aatttttctg attatatttt
atgagttcag aaaggaaatg gtaaaagaac tatacatttt
catgttttaa cattttatgt acgtacttga ttctgtctgt
gtcataatta cacatttact tgaacacagc tatcctttat
cttgtgcttt ctttaataga aaaatgaaca gaaactgaat
gcagttaaat ttttattttt agtaggttgt gaagttactt
ttactggaga aataaaaata tgttaaactt ga

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of Homo sapiens ubiquitin specific peptidase 17 like family member 13 (USP17L13), mRNA having NCBI Reference Sequence:

NM_001256855.1 (SEQ ID NO: 69):
atggaggagg actcactcta cttgggtggt gagtggcagt
tcaaccactt ttcaaaactc acatcttctc ggctcgatgc
agcttttgct gaaatccagc ggacttctct ccctgagaag
tcaccactct catgtgagac ccgtgtcgac ctctgtgatg
atttggttcc tgaggcaaga cagcttgctc ccagggagaa
gcttcctctg agtagcagga gacctgctgc ggtgggggct
gggctccaga atatgggaaa tacctgctac gtgaacgctt
ccttgcagtg cctgacatac acaccgcccc ttgccaacta
catgctgtcc cgggagcact ctcaaacgtg tcatcgtcac
aagggctgca tgctctgtac tatgcaagct cacatcacac
gggccctcca caatcctggc cacgtcatcc agccctcaca
ggcattggct gctggcttcc atagaggcaa gcaggaagat
gcccatgaat ttctcatgtt cactgtggat gccatgaaaa
aggcatgcct tcccgggcac aagcaggtag atcatccctc
taaggacacc accctcatcc accaaatatt tggaggctac
tggagatctc aaatcaagtg tctccactgc cacggcattt
cagacacttt tgacccttac ctggacatcg ccctggatat
ccaggcagct cagagtgtcc agcaagcttt ggaacagttg
gtgaagcccg aagaactcaa tggagagaat gcctatcatt
gtggtgtttg tctccagagg gcgccggcct ccaagacgtt
aactttacac acctctgcca aggtcctcat ccttgtattg
aagagattct ccgatgtgac aggcaacaag attgccaaga
atgtgcaata tcctgagtgc cttgacatgc agccatacat
gtctcagcag aacacaggac ctcttgtcta tgtcctctat
gctgtgctgg tccacgctgg gtggagttgt cacaacggac
attacttctc ttatgtcaaa gctcaagaag gccaatggta
taaaatggat gatgccgagg tcaccgccgc tagcatcact
tctgtcctga gtcaacaggc ctacgtcctc ttttacatcc
agaagagtga atgggaaaga cacagtgaga gtgtgtcaag
aggcagggaa ccaagagccc ttggcgcaga agacacagac
aggcgagcaa cgcaaggaga gctcaagaga gaccacccct
gcctccaggc ccccgagttg gacgagcact tggtggaaag
agccactcag gaaagcacct tagaccgctg gaaattcctt
caagagcaaa acaaaacgaa gcctgagttc aacgtcagaa
aagtcgaagg taccctgcct cccgacgtac ttgtgattca
tcaatcaaaa tacaagtgtg ggatgaagaa ccatcatcct
gaacagcaaa gctccctgct aaacctctct tcgtcgaccc
cgacacatca ggagtccatg aacactggca cactcgcttc
cctgcgaggg agggccagga gatccaaagg gaagaacaaa
cacagcaaga gggctctgct tgtgtgccag tga

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of Homo sapiens mirror-image polydactyly 1 (MIPOL1), transcript variant 1, mRNA having NCBI Reference Sequence:

NM_001195296.2 (SEQ ID NO: 70):
aggccccacg cgccgccccg ctcctccgcc ggatcgtctg
tgggtgagtc tcgagccagg aggctctgag ccagtggcga
ttggctgacg cggtggctgc gcactcggcc tgagaaactc
ggcaagcgcg cagtgtcgac tccccggtct atgccaggcg
catctcagat accagcattg ccaccggtgg gtagaacact
aagtgggctc ttggagtccc tgattccaga acttgactct
tggatgacat ttctggacct gctctgggcc agagaggaga
ccacttccct taagggaacg aggtctcact atattgccca
gactggtctc gaactcctgg gctcaaacag tccccctgcg
ttggcctccc aaagtgatgg aattacaggt gtgaatcact
gcatctgact atggcaagga tctctgtcac tgagctaatc
caaaagtaaa tgagaaactt agaaaaagat tgccaattcc
aaatcaacat atttagagaa aattggaaaa ggagaagctt
actacagctt tatttgagga ctttttaaag aacgctgggt
tctatctgtg agctgcaaat cttggagcaa aaaccagaga
cattgccaga gcaaacaaga acagaaatac aaatggagaa
ctggtcaaaa gacataaccc acagttatct tgaacaagaa
actacgggga taaataaaag tacgcagcca gatgagcaac
tgactatgaa ttctgagaaa agtatgcatc ggaaatccac
tgaattagtt aatgaaataa catgtgagaa cacagaatgg
ccagggcaga gatcaacgaa ttttcagatc atcagttctt
atccagatga tgagtctgtt tactgcacta ctgaaaaata
caacgttatg gaacatagac ataatgatat gcattatgaa
tgtatgactc cttgtcaagt tacttcagac tcagataaag
agaagacaat agcatttctt ctaaaagaat tggatattct
cagaacaagc aataaaaagc ttcagcagaa attggctaaa
gaagataaag aacagagaaa actaaagttt aagctggaac
tccaagagaa agaaacagaa gctaaaattg ctgaaaagac
agcagctctg gttgaagaag tgtattttgc gcagaaggaa
cgtgatgaag ctgttatgtc tagactgcaa ttagccattg
aggagagaga tgaagcaatt gcacgagcca agcatatgga
aatgtctcta aaagtgctag aaaatattaa ccctgaagaa
aatgacatga cattacagga attactgaac agaataaaca
atgcagacac agggatagct attcagaaga atggagctat
aattgtggat agaatctaca agaccaagga atgtaaaatg
agaataactg cagaagaaat gagtgcacta atagaagaac
gggatgctgc cttgtctaag tgcaaacggt tagagcagga
gcttcatcat gtgaaagagc agaaccagac ttcagcaaac
aacatgagac atctgactgc tgaaaacaat caagaacgtg
ctctgaaggc aaagttgtta tctatgcaac aagccagaga
aactgcagtt caacagtaca aaaaactgga agaggaaatc
cagacccttc gagtttacta cagtttacac aaatctttat
ctcaagaaga aaatctgaag gatcagttta actataccct
tagtacatat gaagaagctt taaaaaacag agagaacatt
gtttccatca ctcaacaaca aaatgaggaa ctggctactc
aactgcaaca agctctgaca gagcgagcaa atatggaatt
acaacttcaa catgccagag aggcctccca agtggccaat
gaaaaagttc aaaagttgga aaggctggtg gatgtactga
ggaagaaggt tggaaccggg accatgagga cagtgatctg
attgaaaaaa aacgacagtc tggggaagcg atcacatctg
gtgaccaggc tgcttcattc aacactgtgt aaacaccaaa
gccttaactt agcaaacagt tcttagaagt gggacactcc
aaccacattc caagctgaga taaaatcaaa tcacaaatgt
ttaaccactt tgctgctgac ttgagttatt tatccaaata
tattaactat agacttttac caatgggtag ctataaggtt
acagcttatt ttgtaactat tttatatctc aatatcttta
atataaatct ttttactgag agatcattat agaaacatgt
taaagttggt taggatcata tcttcacata tggccctttc
tgaatcaaag tgcggcaaag taaatattgt ctaagcttta
atccactgtg ttaggtcaaa acttcaaata catgcatttt
tcaatatagg gtatatttct taactgatga gagaggctta
gacatgagtg tgtagtcttc cttcaatgcg tgtatgtaat
ctttgttagt ataaaagata ttaaatatag gtgccaagaa
ttaaatgtat aatttgttta ataagagatg gatatattaa
aattacattc atcaaggcat gatttttgtt tcactacaaa
taatgcaaac tgttttcaat aaaaagagga gactgttaat
gtgtacttat aaattcacat tgtcagtatt ttttaatatt
gggtctgaat aattatctga attctactta agctataagt
ctctgtcatt tttgcttgaa aattagcatg cctctgtctt
aaaagagacc atcaaaccta ttaagtattc ttattgttta
tcttttttta attgccattt gatttttatt gtggaggagg
aggatctaat atataatatt caatacaatt gtaatgtaga
aatataagaa tttagaaaaa agtaaacttg ccatttcgtt
aaggttacct gctttttatt ttattccaga aataagatag
ttacagcata gcagtcatat gaagttatga atagaactga
gatttttatg taattagtta taggcaaaat actttcatat
ttttagatta gacagacgaa agaccaagag gaaatgactg
tgcctggaac aggatgaaat agacaagtag agatttaatt
agcaaaaatt ttgtagggaa gtaaaatttt ttctaagtct
atacgttttt aattcatctt taagattgag ctaaattatc
taccattgcc tatttacagg ataagtacat tcaggacaat
ttattgtacc attctttcat ataccatagc agattatcca
tttcaatttt tttttactcc acaggaaaat gtaagctact
ttgtcataga tcacaaaaga atcataatgc taacaaactc
tatttcttcc ttattaaatc tgtatccatt aaagtaactt
ttttaactat gagaattaga aaataaggga caacaggggt
taaaaataaa ctgatttaat ttgctctata gtcctaaaga
gaattatatc ctctcataga ccaatgactc tataatagag
aaatatgtaa tgatttggtc atgcatggag tcttgtctct
gggctctatt tgaaacgtgt aacagctccc tatgtgaaga
aacattatct ttaaagtcat ctgggaggtg caggtaagta
aagagaaaca atttttgtca caaagtagtc cattcctgat
ctcacttaaa ataaatcaca agtaaatttg aattttcggt
ttaatgttga aagcagatga acttttcttg agttattttg
tcttttagaa tacacaaaat atagtataaa gacatttcac
aatttccaaa caaatctttc tacgcttaaa tgatcaaatt
agaaaaacca attcctataa ttaatatgca gaacttttat
agaatatgat attataaagt taaatttgca aaataattct
aacatccact attgttcagt ataggtaatc tcccaaaaat
atcacatcct cttgaaaatg aattgtctac aaaatttcaa
atgcaaagta ttacagccaa atattatctt aattaatttc
ttacacttaa aagtgtccat caacagtgtg tcagatggtc
tttatatatt ttttctgtat gaaggaatag ccttgcctac
tgaagatagg ttctctatgt attcaacaat aatgcaactt
tagaagtatt ccacactgct gagaaatgtt gctttgagtt
tgctttacac atcatcaact ctaaatccta tagtaacatg
agaattcact tcttcttgta aaaataagta atttacagga
aaggcaaaat gctaatacta acatttgtag cacttgatga
tttacaaagt ccttttacat ctgttttttc atttgatcct
atcgacaatc ataataataa taaattatat atataaccca
tataataacg atggtgatga taattttagt attactgtca
ttataattat tattatattc attctacaga tgaggaaaca
gactcagtaa gccttggaat ttgccaagga ctggctagga
cctagaacta agatcttata accacactcc atgcaactat
gagtctctaa atctctctgt aagaaaaaaa tttcaaagta
aagattttac tcttgcaatt tctgttgtga tagtaccatc
tctttcacat actacataca aattccctaa taatcaaaag
attgtacaca tttttttcaa tgaagtacaa taatgtgaac
tgcatgtgta aacagcatca cattattacc acctctctat
ggtattatag tggcagtatg ctttgcctca gccttcacta
attaggaata ttgtgacaat tcattgctaa taaaacataa
catgagtctc tttgtactgt ttttaatttg gcctgatgct
aaaacctatc agcttagttt tttaacatgg tagtctaaac
ttcaaggctt gctctatctc ctagagcaat ttagagctaa
aaccagtaat aagcaaagtt attggttata agcaaggtat
aagcagttaa agcaaagtta ttaattatta atatatgaga
atttaaaatt ttataaataa gttagaaatt attaatgtat
tttaaatatg gagtaaaata ttttgctaat ttttttcaat
gataaagttg atcctttgta tatttcctta ttcaaataaa
ttcagtcatc ttcttatcag gttgcttatt tatataattt
gtttatttta ttaaaaatct agaatattaa agatttatac
ttttaatatt gaatatagtt tcgtagaaaa acattttata
tgtactattc ttaaagatca caattatttt taaactttct
attttcaaat acttgtgatt aataaggcat tctatgatga
aatagccatg gcttatatat ttttgtcttt tcacatatga
tgtgtttgga acccaaattt tttactgatt ttattgtgtt
gttggtaact agtatttaca gctaacctat ctattcatat
tttacatata tatatatata tatacatgca cacacaccga
tacatttacc ttttaaaaaa atgatttata agtgaactgt
taggctccag tgatttcagt gttttgttca tyttttattg
gaaatgcacg tggtttctta ggcttttaaa caaatacatg
gaatggttga aagatttatt ttgctcgtgc ttagctaaat
atgtcatctc tagaaaagat gtggtttgtt ttggcactgt
tttaaaaact caaatatttt aaacatttgt taagttggag
cttgcacatt taaaccagca gcatacgttc cagaagagca
tctcaagtta ccaaattttt tgtaatcctt ttactagaaa
aatctttttt gaagagtttt tcttgtgctt gcttcggcag
catatatact gaaattagaa aaagaaaact tttctttttt
attatatatt ttagtgtaag ttaagttaaa cttctaaaag
ttgaattatt taatgtagga cttcataaat agtatttgac
aatgataaat gtgtattttt gagaatcatt aaaatgtata
caatgatatt cctttgcaga agtcttaata tgcataattt
ttaatctaat tgtcttctaa atatagtttt ggtgtatgct
ggtattttag aagccaccaa tttctggact atctgattat
taacaaagat gtattttaat gcacaaactc aaatgtttaa
aatatatatt tactttgtac tcaaataaaa tcccctattg
caaatcctat acatatttca atgcaattct tatacatcga
tcttaatcaa atttataaat gtacaatcct gaagacagtg
tactagatat gcactataaa atatattgca gtaaggaaac
agattaagtt cactgtcaaa ctgtctgact tccttaacaa
atttaataaa gataaatgtc aatatgaaca aatttgttag
gtaccttagg aatctttcaa aactatttag atataaaata
aaattatagt aataaaatat tgaagagaaa tatcattttt
agtgaatatg gttagaaaga acataagaag tgagatcaaa
ttattttaat taaaaaaata ggccaggtgc gatggctcac
gcctgtaatc ccagcatttt gggaggccga ggcgggcgga
tcacaaggtc aggagattga gaccatcctg gctaacacgg
tgaaaccccg tctctactaa aaaaagtaca aaaaagaagt
tagccgggcg tggtggcggg cgcctgtagt cccagctact
tgggaggctg aggcaggaga atggcgtgaa cccggaaggc
agagcttgca gtgagccgag atcacgccat tgcactccag
cctgggtgac agagtgagac tgtctcaaat aataataata
ataataataa taataataat aataataata gactgactca
aatctaccat aaagcacctc tactcatttt ggaatatgta
cgtggaattt tgcttcaata gctgaaatct aaaaacactc
tttctgttgc atttgattat tcaatcatga taaggtaaat
gtaaacagac tgtgtagcac tctatcacct ggagtgaact
tagaatttac agcttagagt acatgactaa aagaggaatg
gagtggaagt gggcagtagg tgtctttggg gcaaaaattc
tataaactgg tgttatctaa actatcacat tactttgtta
aattattgtt tcatcacgta tgacctttgt gatttctcac
acattttact tctacatatq cacataattg taattttttt
aactttaaaa agtcaattat gtttaagaaa atttttaaaa
taagaatgaa cgtatgtgat atttactgtg a

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of Homo sapiens ribokinase (RBKS), transcript variant 1, mRNA having NCBI Reference Sequence:

NM_022128.3 (SEQ ID NO: 71):
ccccagaggc agtggcaaga ggaggtggcg gcggtggtag
tggtgggctc ctgcatgacc gacctggtca gtcttacttc
tcgtttgcca aaaactggag aaaccatcca tggacataag
ttttttattg gctttggagg gaaaggtgcc aaccagtgtg
tccaagctgc tcggcttgga gcaatgacgt ccatggtgtg
taaggttggc aaagattctt ttggcaatga ttatatagaa
aacttaaaac agaatgatat ttctacagaa tttacatatc
agactaaaga tgctgctaca ggaactgctt ctataattgt
caataatgaa ggccagaata tcattgtcat agtggctgga
gcaaatttac ttttgaatac ggaggatctg agggcagcag
ccaatgtcat tagcagagcc aaagtcatgg tctgccagct
cgaaataact ccagcaactt ctttggaagc cctaacaatg
gcccgcagga gtggagtgaa aaccttgttc aatccagccc
ctgccattgc tgacctggat ccccagttct acaccctctc
agatgtgttc tgctgcaatg aaagtgaggc tgagatttta
actggcctca cggtgggcag cgctgcagat gctggggagg
ctgcattagt gctcttgaaa aggggctgcc aggtggtaat
cattacctta ggggctgaag gatgtgtggt gctgtcacag
acagaacctg agccaaagca cattcccaca gagaaagtca
aggctgtgga taccacgggt gctggtgaca gctttgtggg
agctctggcc ttctacctgg cttactatcc aaatctgtcc
ttggaagaca tgctcaacag atccaatttc attgcagcag
tcagtgtcca ggctgcagga acacagtcat cttaccctta
caaaaaagac cttccgctta ctctgttttg attgctatta
gtcccaaaat aaatatacct gggaataaaa tgtacttggg
ggtggctgct cctggctaat gcttattaga aaatgtcctc
gtcccctttc tttgcaaata ttagttcttt tacgaagtca
tcctcaagct tcaatttatt tataacgatg attcttttgc
tttccatgca tttgcacaaa acaaccagaa ttaaagattc
cacaaccaag atctgtacaa acataaa

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of Homo sapiens ubiquitin specific peptidase 17 like family member 2 (USP17L2), mRNA having NCBI Reference Sequence:

NM_201402.3 (SEQ ID NO: 72):
gtcatttgaa gactctcttg gaagagatag cgtcttgctg
caacctgcag tcccagcaga aaaaccttgt gatccttgtt
gcgggcgaca tggaggacga ctcactctac ttgggaggtg
agtggcagtt caaccacttt tcaaaactca catcttctcg
gccagatgca gcttttgctg aaatccagcg gacttctctc
cctgagaagt caccactctc atctgaggcc cgtgtcgacc
tctgtgatga tttggctcct gtggcaagac agcttgctcc
caggaagaag cttcctctga gtagcaggag acctgctgcg
gtgggggctg ggctccagaa tatgggaaat acctgctacg
agaacgcttc cctgcagtgc ctgacataca caccgcccct
tgccaactac atgctgtccc gggagcactc tcaaacatgt
cagcgtccca agtgctgcat gctctgtact atgcaagctc
acatcacatg ggccctccac agtcctggtc atgtcatcca
gccctcacag gcattggctg ctggcttcca tagaggcaag
caggaagatg cccatgaatt tctcatgttc actgtggatg
ccatgaaaaa ggcatgcctt cccggccaca agcaggtaga
tcatcactct aaggacacca ccctcatcca ccaaatattt
ggaggctgct ggagatctca aatcaagtgt ctccactgcc
acgggatttc agacactttt gacccttacc tggacatcgc
cctggatatc caggcagctc agagtgtcaa gcaagctttg
gaacagttgg tgaagcccga agaactcaat ggagagaatg
cctatcattg cggtctttgt ctccagaggg cgccggcctc
caagacgtta actttacaca cttctgccaa ggtcctcatc
cttgtcttga agagattctc cgatgtcaca ggcaacaaac
ttgccaagaa tgtgcaatat cctgagtgcc ttgacatgca
gccatacatg tctcagcaga acacaggacc tcttgtctat
gtcctctatg ctgtgctggt ccacgctggg tggagttgtc
acgacggaca ttacttctct tatgtcaaag ctcaagaagg
ccagtggtat aaaatggatg atgccaaggt cactgcctgt
agcatcactt ctgtcctgag tcaacaggcc tatgtcctct
tttacatcca gaagagtgaa tgggaaagac acagtgagag
tgtgtcaaga ggcagggaac caagagccct cggcgctgaa
gacacagaca ggcgagcaac gcaaggagag ctcaagagag
accacccctg cctccaggca cccgagttgg acgagcgctt
ggtggaaaga gccactcagg aaagcacctt agaccactgg
aaattccccc aagagcaaaa caaaacgaag cctgagttca
acgtcagaaa agtcgaaggt accctgcctc ccaacgtact
tgtgattcat caatcgaaat acaagtgtgg gatgaaaaac
catcatcctg aacagcaaag ctccctgcta aacctctctt
cgacgacccg gacagatcag gagtccgtga acactggcac
cctcgcttct ctgcaaggga ggaccaggag atccaaaggg
aagaacaaac acagcaagag ggctctgctt gtgtgccagt
gatctcagtg gaagtgccga cccacacgta ggggtgaacg
cacacacaca cacgcacaaa tacacccaca agcgcgcacg
caaacacaca cacacacaca aacacgaaca ccgtcaatcc
tacataaagt aatgaggagt ccaagtttct gtctctacaa
cagggacaac tggatagtga tggctgcatc tcaggatgag
cccacacatg ggaaacatca agttttgggg tcgtgagtct
tccgaacctc tggagagact gtctgtgtgt ttgtgttcat
ggtagatgac attcactgtg tatttctgaa tatgacctac
tgacgtgtag gtttgagtgt gaggttattg caggggactc
ggtttcctat tttctcttgg ggtgtgtttc attcgtcagt
tgttgggcgg cacgagaagg tgaaattttg ctcatgtggc
acatccatgg atcattctcg ccaccttgaa tagtggaaac
tggaatgcat ttagaagata ggaacggtgc tcttctttct
taccctggct caccgttttt acattggttt ctgaatggac
ctcaggcgcc ctgggacttg tgctcttgct ggaacccaca
taacgccgga aacagacaga ccgacttgcc tgtttcacga
tgtccaattc caatgagtcg aaatggaaaa ttttcccact
ggcatgtcag tcatttggaa ataagtcgta ttgataataa
aggaaatcaa acaca

a different transcript variant of the gene, or a sequence with at least 80%, 82%, 85%, 86%, 88%, 90%, 92%, 94%, 95%, 97%, 98% or 99% nucleic acid sequence identity thereto.

In embodiments, the nucleic acid inhibits expression of Homo sapiens dystrophin (DMD), transcript variant Dp427m, mRNA having NCBI Reference Sequence:

NM_004006.3 (SEQ ID NO: 73):
atcagttact gtgttgactc actcagtgtt gggatcactc
actttccccc tacaggactc agatctggga ggcaattacc
ttcggagaaa aacgaatagg aaaaactgaa gtgttacttt
ttttaaagct gctgaagttt gttggtttct cattgttttt
aagcctactg gagcaataaa gtttgaagaa cttttaccag
gtttttttta tcgctgcctt gatatacact tttcasaatg
ctttggtggg aagaagtaga ggactgttat gaaagagaag
atgttcaaaa gaaaacattc acaaaatggg taaatgcaca
attttctaag tttgggaagc agcatattga gaacctcttc
agtgacctac aggatgggag gcgcctccta gacctcctcg
aaggcctgac agggcaaaaa ctgccaaaag aaaaaggatc
cacaagagtt catgccctga acaatgtcaa caaggcactg
cgggttttgc agaacaataa tgttgattta gtgaatattg
gaagtactga catcgtagat ggaaatcata aactgactct
tggtttgatt tggaatataa tcctccactg gcaggtcaaa
aatgtaatga aaaatatcat ggctggattg caacaaacca
acagtgaaaa gattctcctg agctgggtcc gacaatcaac
tcgtaattat ccacaggtta atgtaatcaa cttcaccacc
agctggtctg atggcctggc tttgaatgct ctcatccata
gtcataggcc agacctattt gactggaata gtgtggtttg
ccagcagtca gccacacaac gactggaaca tgcattcaac
atcgccagat atcaattagg catagagaaa ctactcgatc
ctgaagatgt tgataccacc tatccagata agaagtccat
cttaatgtac atcacatcac tcttccaagt tttgcctcaa
caagtgagca ttgaagccat ccaggaagtg gaaatgttgc
caaggccacc taaagtgact aaagaagaac attttcagtt
acatcatcaa atgcactatt ctcaacagat cacggtcagt
ctagcacagg gatatgagag aacttcttcc cctaagcctc
gattcaagag ctatgcctac acacaggctg cttatgtcac
cacctctgac cctacacgga gcccatttcc ttcacagcat
ttggaagctc ctgaagacaa gtcatttggc agttcattga
tggagagtga agtaaacctg gaccgttatc aaacagcttt
agaagaagta ttatcgtggc ttctttctgc tgaggacaca
ttgcaagcac aaggagagat ttctaatgat gtggaagtgg
tgaaagacca gtttcatact catgaggggt acatgatgga
tttgacagcc catcagggcc gggttggtaa tattctacaa
ttgggaagta agctgattgg aacaggaaaa ttatcagaag
atgaagaaac tgaagtacaa gagcagatga atctcctaaa
ttcaagatgg gaatgcctca gggtagctag catggaaaaa
caaagcaatt tacatagagt tttaatggat ctccagaatc
agaaactgaa agagttgaat gactggctaa caaaaacaga
agaaagaaca aggaaaatgg aggaagagcc tcttggacct
gatcttgaag acctaaaacg ccaagtacaa caacataagg
tgcttcaaga agatctagaa caagaacaag tcagggtcaa
ttctctcact cacatggtgg tggtagttga tgaatctagt
ggagatcacg caactgctgc tttggaagaa caacttaagg
tattgggaga tcgatgggca aacatctgta gatggacaga
agaccgctgg gttcttttac aagacatcct tctcaaatgg
caacgtctta ctgaagaaca gtgccttttt agtgcatggc
tttcagaaaa agaagatgca gtgaacaaga ttcacacaac
tggctttaaa gatcaaaatg aaatgttatc aagtcttcaa
aaactggccg ttttaaaagc ggatctagaa aagaaaaagc
aatccatggg caaactgtat tcactcaaac aagatcttct
ttcaacactg aagaataagt cagtgaccca gaagacggaa
gcatggctgg ataactttgc ccggtcttgg gataatttag
tccaaaaact tgaaaagagt acagcacaga tttcacaggc
tgtcaccacc actcagccat cactaacaca gacaactgta
atggaaacag taactacggt gaccacaagg gaacagatcc
tggtaaagca tgctcaagag gaacttccac caccacctcc
ccaaaagaag aggcagatta ctgtggattc tgaaattagg
aaaaggttgg atgttgatat aactgaactt cacagctgga
ttactcgctc agaagctgtg ttgcagagtc ctgaatttgc
aatctttcgg aaggaaggca acttctcaga cttaaaagaa
aaagtcaatg ccatagagcg agaaaaagct gagaagttca
gaaaactgca agatgccagc agatcagctc aggccctggt
ggaacagatg gtgaatgagg gtgttaatgc agatagcatc
aaacaagcct cagaacaact gaacagccgg tggatcgaat
tctgccagtt gctaagtgag agacttaact ggctggagta
tcagaacaac atcatcgctt tctataatca gctacaacaa
ttggagcaga tgacaactac tgctgaaaac tggttgaaaa
tccaacccac caccccatca gagccaacag caattaaaag
tcagttaaaa atttgtaagg atgaagtcaa ccggctatca
gatcttcaac ctcaaattga acgattaaaa attcaaagca
tagccctgaa agagaaagga caaggaccca tgttcctgga
tgcagacttt gtggccttta caaatcattt taagcaagtc
ttttctgatg tgcaggccag agagaaagag ctacagacaa
tttttgacac tttgccacca atgcgctatc aggagaccat
gagtgccatc aggacatggg tccagcagtc agaaaccaaa
ctctccatac ctcaacttag tgtcaccgac tatgaaatca
tggagcagag actcggggaa ttgcaggctt tacaaagttc
tctgcaagag caacaaagtg gcctatacta tctcagcacc
actgtgaaag agatgtcgaa gaaagcgccc tctgaaatta
gccggaaata tcaatcagaa tttgaagaaa ttgagggacg
ctggaagaag ctctcctccc agctggttga gcattgtcaa
aagctagagg agcaaatgaa taaactccga aaaattcaga
atcacataca aaccctgaag aaatggatgg ctgaagttga
tgtttttctg aaggaggaat ggcctgccct tggggattca
gaaattctaa aaaagcagct gaaacagtgc agacttttag
tcagtgatat tcagacaatt cagcccagtc taaacagtgt
caatgaaggt gggcagaaga taaagaatga agcagagcca
gagtttgctt cgagacttga gacagaactc aaagaactta
acactcagtg ggatcacatg tgccaacagg tctatgccag
aaaggaggcc ttgaagggag gtttggagaa aactgtaagc
ctccagaaag atctatcaga gatgcacgaa tggatgacac
aagctgaaga agagtatctt gagagagatt ttgaatataa
aactccagat gaattacaga aagcagttga agagatgaag
agagctaaag aagaggccca acaaaaagaa gcgaaagtga
aactccttac tgagtctgta aatagtgtca tagctcaagc
tccacctgta gcacaagagg ccttaaaaaa ggaacttgaa
actctaacca ccaactacca gtggctctgc actaggctga
atgggaaatg caagactttg gaagaagttt gggcatgttg
gcatgagtta ttgtcatact tggagaaagc aaacaagtgg
ctaaatgaag tagaatttaa acttaaaacc actgaaaaca
ttcctggcgg agctgaggaa atctctgagg tgctagattc
acttgaaaat ttgatgcgac attcagagga taacccaaat
cagattcgca tattggcaca gaccctaaca gatggcggag
tcatggatga gctaatcaat gaggaacttg agacatttaa
ttctcgttgg agggaactac atgaagaggc tgtaaggagg
caaaagttgc ttgaacagag catccagtct gcccaggaga
ctgaaaaatc cttacactta atccaggagt ccctcacatt
cattgacaag cagttggcag cttatattgc agacaaggtg
gacgcagctc aaatgcctca ggaagcccag aaaatccaat
ctgatttgac aagtcatgag atcagtttag aagaaatgaa
gaaacataat caggggaagg aggctgccca aagagtcctg
tctcagattg atgttgcaca gaaaaaatta caagatgtct
ccatgaagtt tcgattattc cagaaaccag ccaattttga
gcagcgtcta caagaaagta agatgatttt agatgaagtg
aagatgcact tgcctgcatt ggaaacaaag agtgtggaac
aggaagtagt acagtcacag ctaaatcatt gtgtgaactt
gtataaaagt ctgagtgaag tgaagtctga agtggaaatg
gtgataaaga ctggacgtca gattgtacag aaaaagcaga
cggaaaatcc caaagaactt gatgaaagag taacagcttt
gaaattgcat tataatgagc tgggagcaaa ggtaacagaa
agaaagcaac agttggagaa atgcttgaaa ttgtcccgta
agatgcgaaa ggaaatgaat gtcttgacag aatggctggc
agctacagat atggaattga caaagagatc agcagttgaa
ggaatgccta gtaatttgga ttctgaagtt gcctggggaa
aggctactca aaaagagatt gagaaacaga aggtgcacct
gaagagtatc acagaggtag gagaggcctt gaaaacagtt
ttgggcaaga aggagacgtt ggtggaagat aaactcagtc
ttctgaatag taactggata gctgtcacct cccgagcaga
agagtggtta aatcttttgt tggaatacca gaaacacatg
gaaacttttg accagaatgt ggaccacatc acaaagtgga
tcattcaggc tgacacactt ttggatgaat cagagaaaaa
gaaaccccag caaaaagaag acgtgcttaa gcgtttaaag
gcagaactga atgacatacg cccaaaggtg gactctacac
gtgaccaagc agcaaacttg atggcaaacc gcggtgacca
ctgcaggaaa ttagtagagc cccaaatctc agagctcaac
catcgatttg cagccatttc acacagaatt aagactggaa
aggcctccat tcctttgaag gaattggagc agtttaactc
agatatacaa aaattgcttg aaccactgga ggctgaaatt
cagcaggggg tgaatctgaa agaggaagac ttcaataaag
atatgaatga agacaatgag ggtactgtaa aagaattgtt
gcaaagagga gacaacttac aacaaagaat cacagatgag
agaaagcgag aggaaataaa gataaaacag cagctgttac
agacaaaaca taatgctctc aaggatttga ggtctcaaag
aagaaaaaag gctctagaaa tttctcatca gtggtatcag
tacaagaggc aggctgatga tctcctgaaa tgcttggatg
acattgaaaa aaaattagcc agcctacctg agcccagaga
tgaaaggaaa atadaggaaa ttgatcggga attgcagaag
aagaaagagg agctgaatgc agtgcgtagg caagctgagg
gcttgtctga ggatggggcc gcaatggcag tggagccaac
tcagatccag ctcagcaagc gctggcggga aattgagagc
aaatttgctc agtttcgaag actcaacttt gcacaaattc
acactgtccg tgaagaaacg atgatggtga tgactgaaga
catgcctttg gaaatttctt atgtgccttc tacttatttg
actgaaatca ctcatgtctc acaagcccta ttagaagtgg
aacaacttct caatgctcct gacctctgty ctaaggactt
tgaagatctc tttaagcaag aggagtctct gaagaatata
aaagatagtc tacaacaaag ctcaggtcgg attgacatta
ttcatagcaa gaagacagca gcattgcaaa gtgcaacgcc
tctggaaagg gtgaagctac aggaagctct ctcccagctt
gatttccaat gggaaaaagt taacaaaatg tacaaggacc
gacaagggcg atttgacaga tctgttgaga aatggcggcg
ttttcattat gatataaaga tatttaatca gtggctaaca
gaagctgaac agtttctcag aaagacacaa attcctgaga
attgggaaca tgctaaatac aaatggtatc ttaaggaact
ccaggatggc attgggcagc ggcaaactgt tgtcagaaca
ttgaatgcaa ctggggaaga aataattcag caatcctcaa
aaacagatgc cagtattcta caggaaaaat tgggaagcct
gaatctgcgg tygcaggagg tctgcaaaca gctgtcagac
agaaaaaaga ggctagaaga acaaaagaat atcttgtcag
aatttcaaag agatttaaat gaatttgttt tatggttgga
ggaagcagat aacattgcta gtatcccact tgaacctgga
aaagagcagc aactaaaaga aaagcttgag caagtcaagt
tactggtgga agagttgccc ctgcgccagg gaattctcaa
acaattaaat gaaactggag gacccgtgct tgtaagtgct
cccataagcc cagaagagca agataaactt gaaaataagc
tcaagcagac aaatctccag tggataaagg tttccagagc
tttacctgag aaacaaggag aaattgaagc tcaaataaaa
gaccttgggc agcttgaaaa aaagcttgaa gaccttgaag
agcagttaaa tcatctgctg ctgtggttat ctcctattag
gaatcagttg gaaatttata accaaccaaa ccaagaagga
ccatttgacg ttaaggaaac tgaaatagca gttcaagcta
aacaaccgga tgtggaagag attttgtcta aagggcagca
tttgtacaag gaaaaaccag ccactcagcc agtgaagagg
aagttagaag atctgagctc tgagtggaag gcggtaaacc
gtttacttca agagctgagg gcaaagcagc ctgacctagc
tcctggactg accactattg gagcctctcc tactcagact
gttactctgg tgacacaacc tgtggttact aaggaaactg
ccatctccaa actagaaatg ccatcttcct tgatgttgga
ggtacctgct ctggcagatt tcaaccgggc ttggacagaa
cttaccgact ggctttctct gcttgatcaa gttataaaat
cacagagggt gatggtgggt gaccttgagg atatcaacga
gatgatcatc aagcagaagg caacaatgca ggatttggaa
cagaggcgtc cccagttyga agaactcatt accgctgccc
aaaatttgaa aaacaagacc agcaatcaag aggctagaac
aatcattacg gatcgaattg aaagaattca gaatcagtgg
gatgaagtac aagaacacct tcagaaccgg aggcaacagt
tgaatgaaat gttaaaggat tcaacacaat ggctggaagc
taaggaagaa gctgagcagg tcttaggaca ggccagagcc
aagcttgagt catggaagga gggtccctat acagtagatg
caatccaaaa gaaaatcaca gaaaccaagc agttggccaa
agacctccgc cagtggcaga caaatgtaga tgtggcaaat
gacttggccc tgaaacttct ccgggattat tctgcagatg
ataccagaaa agtccacatg ataacagaga atatcaatgc
ctcttggaga agcattcata aaagggtgag tgagcgagag
gctgctttgg aagaaactca tagattactg caacagttcc
ccctggacct ggaaaagttt cttgcctggc ttacagaagc
tgaaacaact gccaatgtcc tacaggatgc tacccgtaag
gaaaggctcc tagaagactc caagggagta aaagagctga
tgaaacaatg gcaagacctc caaggtgaaa ttgaagctca
cacagatgtt tatcacaacc tggatgaaaa cagccaaaaa
atcctgagat ccctggaagg ttccgatgat gcagtcctgt
tacaaagacg tttggataac atgaacttca agtggagtga
acttcggaaa aagtctctca acattaggtc ccatttggaa
gccagttctg accagtggaa gcgtctgcac ctttctctgc
aggaacttct ggtgtggcta cagctgaaag atgatgaatt
aagccggcag gcacctattg gaggcgactt tccagcagtt
cagaagcaga acgatgtaca tagggccttc aagagggaat
tgaaaactaa agaacctgta atcatgagta ctcttgagac
tctacgaata tttctgacag agcagccttt ggaaggacta
gagaaactct accaggagcc cagagagctg cctcctgagg
agagagccca gaatgtcact cggcttctac gaaagcaggc
tgaggaggtc aatactgagt gggaaaaatt gaacctgcac
tccgctgact ggcagagaaa aatagatgag acccttgaaa
gactccggga acttcaagag gccacggatg agctggacct
caagctgcgc caagctgagg tgatcaaggg atcctggcag
cccgtgggcg atctcctcat tgactctctc caagatcacc
tcgagaaagt caaggcactt cgaggagaaa ttgcgcctct
gaaagagaac gtgagccacg tcaatgacct tgctcgccag
cttaccactt tgggcattca gctctcaccg tataacctca
gcactctgga agacctgaac accagatgga agcttctgca
ggtggccgtc gaggaccgag tcaggcagct gcatgaagcc
cacagggact ttggtccagc atctcagcac tttctttcca
cgtctgtcca gggtccctgg gagagagcca tctcgccaaa
caaagtgccc tactatatca accacgagac tcaaacaact
tgctgggacc atcccaaaat gacagagctc taccagtctt
tagctgacct gaataatgtc agattctcag cttataggac
tgccatgaaa ctccgaagac tgcagaaggc cctttgcttg
gatctcttga gcctgtcagc tgcatgtgat gccttggacc
agcacaacct caagcaaaat gaccagccca tggatatcct
gcagattatt aattgtttga ccactattta tgaccgcctg
gagcaagagc acaacaattt ggtcaacgtc cctctctgcg
tggatatgtg tctgaactgg ctgctgaatg tttatgatac
gggacgaaca gggaggatcc gtgtcctgtc ttttaaaact
ggcatcattt ccctgtgtaa agcacatttg gaagacaagt
acagatacct tttcaagcaa gtggcaagtt caacaggatt
ttgtgaccag cgcaggctgg gcctccttct gcatgattct
atccaaattc caagacagtt gggtgaagtt gcatcctttg
ggggcagtaa cattgagcca agtgtccgga gctgcttcca
atttgctaat aataagccag agatcgaagc ggccctcttc
ctagactgga tgagactgga accccagtcc atggtgtggc
tgcccgtcct gcacagagtg gctgctgcag aaactgccaa
gcatcaggcc aaatgtaaca tctgcaaaga gtgtccaatc
attggattca ggtacaggag tctaaagcac tttaattatg
acatctgcca aagctgcttt ttttctggtc gagttgcaaa
aggccataaa atgcactatc ccatggtgga atattgcact
ccgactacat caggagaaga tgttcgagac tttgccaagg
tactaaaaaa caaatttcga accaaaaggt attttgcgaa
gcatccccga atgggctacc tgccagtgca gactgtctta
gagggggaca acatggaaac tcccgttact ctgatcaact
tctggccagt agattctgcg cctgcctcgt cccctcagct
ttcacacgat gatactcatt cacgcattga acattatgct
agcaggctag cagaaatgga aaacagcaat ggatcttatc
taaatgatag catctctcct aatgagagca tagatgatga
acatttgtta atccagcatt actgccaaag tttgaaccag
gactcccccc tgagccagcc tcgtagtcct gcccagatct
tgatttcctt agagagtgag gaaagagggg agctagagag
aatcctagca gatcttgagg aagaaaacag gaatctgcaa
gcagaatatg accgtctaaa gcagcagcac gaacataaag
gcctgtcccc actgccgtcc cctcctgaaa tgatgcccac
ctctccccag agtccccggg atgctgagct cattgctgag
gccaagctac tgcgtcaaca caaaggccgc ctggaagcca
ggatgcaaat cctggaagac cacaataaac agctggagtc
acagttacac aggctaaggc agctgctgga gcaaccccag
gcagaggcca aagtgaatgg cacaacggtg tcctctcctt
ctacctctct acagaggtcc gacagcagtc agcctatgct
gctccgagtg gttggcagtc aaacttcgga ctccatgggt
gaggaagatc ttctcagtcc tccccaggac acaagcacag
ggttagagga ggtgatggag caactcaaca actccttccc
tagttcaaga ggaagaaata cccctggaaa gccaatgaga
gaggacacaa tgtaggaagt cttttccaca tygcagatga
tttgggcaga gcgatggagt ccttagtatc agtcatgaca
gatgaagaag gagcagaata aatgttttac aactcctgat
tcccgcatgg tttttataat attcatacaa caaagaggat
tagacagtaa gagtttacaa gaaataaatc tatatttttg
tgaagggtag tggtattata ctgtagattt cagtagtttc
taagtctgtt attgttttgt taacaatggc aggttttaca
cgtctatgca attgtacaaa aaagttataa gaaaactaca
tgtaaaatct tgatagctaa ataacttgcc attttttaat
atggaacgca ttttgggttg tttaaaaatt tataacagtt
ataaagaaag attgtaaact aaagtgtgct ttataaaaaa
aagttgttta taaaaacccc taaaaacaaa acaaacacac
acacacacac atacacacac acacacaaaa ctttgaggca
gcgcattgtt ttgcatcctt ttggcgtgat atccatatga
aattcatggc tttttctttt tttgcatatt aaagataaga
cttcctctac caccacacca aatgactact acacactgct
catttgagaa ctgtcagctg agtggggcag gcttgagttt
tcatttcata tatctatatg tctataagta tataaatact
atagttatat agataaagag atacgaattt ctatagactg
actttttcca ttttttaaat gttcatgtca catcctaata
gaaagaaatt acttctagtc agtcatccag gcttacctgc
ttggtctaga atggattttt cccggagccg gaagccagga
ggaaactaca ccacactaaa acattgtcta cagctccaga
tgtttctcat tttaaacaac tttccactga caacgaaagt
aaagtaaagt attggatttt tttaaaggga acatgtgaat
gaatacacag gacttattat atcagagtga gtaatcggtt
ggttggttga ttgattgatt gattgataca ttcagcttcc
tgctgctagc aatgccacga tttagattta atgatgcttc
agtggaaatc aatcagaagg tattctgacc ttgtgaacat
cagaaggtat tttttaactc ccaagcagta gcaggacgat
gatagggctg gagggctatg gattcccagc ccatccctgt
gaaggagtag gccactcttt aagtgaagga ttggatgatt
gttcataata cataaagttc tctgtaatta caactaaatt
attatgccct cttctcacag tcaaaaggaa ctgggtggtt
tggtttttgt tgctttttta gatttattgt cccatgtggg
atgagttttt aaatgccaca agacataatt taaaataaat
aaactttggg aaaaggtgta aaacagtagc cccatcacat
ttgtgatact gacaggtatc aacccagaag cccatgaact
gtgtttccat cctttgcatt tctctgcgag tagttccaca
caggtttgta agtaagtaag aaagaaggca aattgattca
aatgttacaa aaaaaccctt cttggtggat tagacaggtt
aaatatataa acaaacaaac aaaaattgct caaaaaagag
gagaaaagct caagaggaaa agctaaggac tggtaggaaa
aagctttact ctttcatgcc attttatttc tttttgattt
ttaaatcatt cattcaatag ataccaccgt gtgacctata
attttgcaaa tctgttacct ctgacatcaa gtgtaattag
cttttggaga gtgggctgac atcaagtgta attagctttt
ggagagtggg ttttgtccat tattaataat taattaatta
acatcaaaca cggcttctca tgctatttct acctcacttt
ggttttgggg tgttcctgat aattgtgcac acctgagttc
acagcttcac cacttgtcca ttgcgttatt ttctttttcc
tttataattc tttctttttc cttcataatt ttcaaaagaa
aacccaaagc tctaaggtaa caaattacca aattacatga
agatttggtt tttgtcttgc atttttttcc tttatgtgac
gctggacctt ttctttaccc aaggattttt aaaactcaga
tttaaaacaa ggggttactt tacattctac taagaagttt
aagtaagtaa gtttcattct aaaatcagag gtaaatagag
tgcataaata attttgtttt aatctttttg tttttctttt
agacacatta gctctggagt gagtctgtca taatatttga
acaaaaattg agagctttat tgctgcattt taagcataat
taatttggac attatttcgt gttgtgttct ttataaccac
caagtattaa actgtaaatc ataatgtaac tgaagcataa
acatcacatg gcatgttttg tcattgtttt caggtactga
gttcttactt gagtatcata atatattgtg ttttaacacc
aacactgtaa catttacgaa ttattttttt aaacttcagt
tttactgcat tttcacaaca tatcagactt caccaaatat
atgccttact attgtattat agtactgctt tactgtgtat
ctcaataaag cacgcagtta tgttacaaaa aa

a different transcript variant of the gene, or a sequence with at least 80%, 82%, 85%, 86%, 88%, 90%, 92%, 94%, 95%, 97%, 98% or 99% nucleic acid sequence identity thereto.

The amino acid sequences for the polypeptides of SEQ ID NOS: 74-91 and the corresponding nucleic acid sequences of SEQ ID NOs: 92-109 that encode the polypeptides of SEQ ID NOS: 74-91 are provided in FIG. 25.

The invention will be described by the following non-limiting example.

### Example 1

Most genetic approaches to identify host factors regulating infection have relied upon loss-of-function screens. Knock-out screens are limited in genes they can query, as genes essential for cell survival cannot be investigated. Moreover, existing screens often rely on proxy phenotypes instead of directly measuring viral replication. This leaves a large amount of genetic space unexplored and raises the possibility that entirely new classes of viral co-factors have yet to be discovered. TRPPC overcomes this in at least 3 ways: 1) it is a fitness-based screen dependent on viral replication; 2) TRPPC inherently rank orders host factors, as the abundance of any particular virus reflects the importance of the modulated host gene; and 3) TRPPC can be used for both loss- and gain-of-function screening, exploring new genetic space including essential genes. Furthermore, this system is entirely portable, functioning with any pathogen that can deliver a targeting RNA, amenable to various iterations changing the selective pressure or modes of replication to focus on different aspects of infection, and in principle can also be performed in vivo in transgenic animals expressing the CRISPRa/i machinery.

TRPPC can be used to identify host factors regulating pathogen replication. The top hits identified by the inventors increase replication of influenza virus, and this information can be used to increase virus yield in commercial settings, and even a modest gain in viral yield would have large impacts on production. Similarly, adenovirus-based vaccines like the adenovirus based COVID19 vaccine are produced in cell culture, and engineering host gene expression to increase yields would have a major impact on this process.

Rank-ordered top hits from influenza virus TRPPC screen in human lung cells:

| | |
|---|---|
| 1. | SLC9C1 (SEQ ID NO: 1) |
| 2. | TICRR (SEQ ID NO: 2) |
| 3. | OR4C6 (SEQ ID NO: 3) |
| 4. | CLEC4C (SEQ ID NO: 4) |
| 5. | NDUFA7 (SEQ ID NO: 5) |
| 6. | OR51A7 (SEQ ID NO: 6) |
| 7. | CLCNKB (SEQ ID NO: 7) |
| 8. | GNG5 (SEQ ID NO: 8) |
| 9. | TYW1 (SEQ ID NO: 9) |
| 10. | RAB42 (SEQ ID NO: 10) |
| 11. | HCN3 (SEQ ID NO: 11) |
| 12. | RASAL1 (SEQ ID NO: 12) |
| 13. | ULBP1 (SEQ ID NO: 13) |
| 14. | C5orf30 (SEQ ID NO: 14) |
| 15. | PARP15 (SEQ ID NO: 15) |
| 16. | NLGN4X (SEQ ID NO: 16) |
| 17. | CD59 (SEQ ID NO: 17) |
| 18. | CFL2 (SEQ ID NO: 18) |
| 19. | GSDMB (SEQ ID NO: 19) |
| 20. | BRD4 (SEQ ID NO: 20) |
| 21. | IFIT3 (SEQ ID NO: 21) |
| 22. | OGFR (SEQ ID NO: 22) |
| 23. | SDR39U1 (SEQ ID NO: 23) |
| 24. | RIMS2 (SEQ ID NO: 24) |
| 25. | ST8SIA3 (SEQ ID NO: 25) |
| 26. | CDKN3 (SEQ ID NO: 26) |
| 27. | TIMD4 (SEQ ID NO: 27) |
| 28. | SYS1 (SEQ ID NO: 28) |
| 29. | UBD (SEQ ID NO: 29) |
| 30. | MED17 (SEQ ID NO: 30) |
| 31. | PEX13 (SEQ ID NO: 31) |
| 32. | USP17L13 (SEQ ID NO: 32) |
| 33. | MIPOL1 (SEQ ID NO: 33) |
| 34. | RBKS (SEQ ID NO: 34) |
| 35. | USP17L2 (SEQ ID NO: 35) |
| 36. | DMD (SEQ ID NO: 36) |

Additional hits are provided in amino acid sequences of SEQ ID NOs: 74-91 shown in FIG. 25.

TABLE 1

| | | | | | | log2 |
|---|---|---|---|---|---|---|
| | RRA | log10 | | | | fold- |
| id | MAGeCK | RRA | p-value | FDR | Rank | change |
| SLC9C1 | 4.58E−10 | −9.34 | 2.90E−06 | 0.001238 | 1 | 7.2241 |
| TICRR | 9.17E−09 | −8.04 | 2.90E−06 | 0.001238 | 2 | 6.107 |
| TREX1 | 6.10E−07 | −6.21 | 2.90E−06 | 0.001238 | 3 | 5.4596 |
| OR4C6 | 7.10E−07 | −6.15 | 2.90E−06 | 0.001238 | 4 | 5.48 |
| CLEC4C | 1.22E−06 | −5.91 | 8.71E−06 | 0.002475 | 5 | 4.4016 |
| NDUFA7 | 1.55E−06 | −5.81 | 8.71E−06 | 0.002475 | 6 | 4.0794 |
| OR51A7 | 1.83E−06 | −5.74 | 1.45E−05 | 0.00275 | 7 | 5.5662 |
| CLCNKB | 1.93E−06 | −5.71 | 1.45E−05 | 0.00275 | 8 | 5.5126 |
| GNG5 | 2.49E−06 | −5.60 | 1.45E−05 | 0.00275 | 9 | 4.1712 |
| TYW1 | 3.75E−06 | −5.43 | 2.03E−05 | 0.003465 | 10 | 3.5509 |
| RAB42 | 5.37E−06 | −5.27 | 3.19E−05 | 0.00495 | 11 | 4.2745 |
| HCN3 | 7.41E−06 | −5.13 | 3.77E−05 | 0.005363 | 12 | 3.659 |
| RASAL1 | 8.19E−06 | −5.09 | 4.94E−05 | 0.005611 | 13 | 3.7478 |
| ULBP1 | 8.74B−06 | −5.06 | 4.94E−05 | 0.005611 | 14 | 3.8628 |
| C5orf30 | 9.31E−06 | −5.03 | 4.94E−05 | 0.005611 | 15 | 4.2296 |
| PARP15 | 1.15E−05 | −4.94 | 6.10E−05 | 0.006188 | 16 | 4.1313 |
| NLGN4X | 1.22E−05 | −4.91 | 6.68E−05 | 0.006188 | 17 | 4.0686 |
| CD59 | 1.29E−05 | −4.89 | 6.68E−05 | 0.006188 | 18 | 4.0867 |
| CFL2 | 1.48E−05 | −4.83 | 7.26E−05 | 0.006188 | 19 | 2.9664 |
| GSDMB | 1.50E−05 | −4.82 | 8.42E−05 | 0.006664 | 20 | 5.4131 |
| BRD4 | 1.52E−05 | −4.82 | 7.26E−05 | 0.006188 | 21 | 4.1823 |
| IFIT3 | 1.87E−05 | −4.73 | 9.00E−05 | 0.006664 | 22 | 4.4612 |
| OGFR | 2.43E−05 | −4.61 | 9.58E−05 | 0.006664 | 23 | 2.9749 |
| SDR39U1 | 2.49E−05 | −4.60 | 9.58E−05 | 0.006664 | 24 | 2.7017 |
| RIMS2 | 2.54E−05 | −4.59 | 0.00010162 | 0.006664 | 25 | 3.1111 |
| ST8SIA3 | 2.67E−05 | −4.57 | 0.00010162 | 0.006664 | 26 | 1.8644 |
| CDKN3 | 3.36E−05 | −4.47 | 0.00012485 | 0.007682 | 27 | 2.7467 |
| TIMD4 | 3.65E−05 | −4.44 | 0.00013066 | 0.007682 | 28 | 2.4562 |
| SYS1 | 3.79E−05 | −4.42 | 0.00013066 | 0.007682 | 29 | 2.8394 |
| UBD | 4.34E−05 | −4.36 | 0.00014808 | 0.008416 | 30 | 4.9229 |
| MED17 | 4.97E−05 | −4.30 | 0.00017131 | 0.009422 | 31 | 4.8446 |
| PEX13 | 5.30E−05 | −4.28 | 0.00018292 | 0.009746 | 32 | 2.4674 |
| USP17L13 | 7.28E−05 | −4.14 | 0.00031068 | 0.015983 | 33 | 3.9451 |
| MIPOL1 | 7.51E−05 | −4.12 | 0.00032229 | 0.015983 | 34 | 2.4368 |
| RBKS | 7.99E−05 | −4.10 | 0.0003281 | 0.015983 | 35 | 2.2546 |
| USP17L2 | 9.01E−05 | −4.05 | 0.00036294 | 0.017189 | 36 | 3.1472 |
| DMD | 9.97E−05 | −4.00 | 0.00038036 | 0.017527 | 37 | 1.8293 |

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A nucleic acid vector comprising a heterologous promoter operably linked to an open reading frame encoding a polypeptide having at least 80% amino acid sequence identity to one of SEQ ID Nos. 1-36 or 74-91 or a portion thereof with the activity of SEQ ID Nos. 1-36 or 74-91.

2. The vector of claim 1, wherein the promoter is a viral promoter.

3. The vector of claim 1, wherein the polypeptide has at least 90% or 95% amino acid sequence identity to one of SEQ ID Nos. 1-36 or 74-91 or the portion thereof with the activity of SEQ ID Nos. 1-36 or 74-91.

4. The vector of claim 1, which is a viral vector or a plasmid.

5. A host cell having the vector of claim 1, or wherein the genome of the host cell is augmented with a nucleic acid encoding a polypeptide having at least 80% amino acid sequence identity to one of SEQ ID Nos. 1-36 or 74-91 or a portion thereof with the activity of SEQ ID Nos. 1-36 or 74-91, or comprising a polypeptide having at least 80% amino acid sequence identity to one of SEQ ID Nos. 1-36 or 74-91 or a portion thereof with the activity of SEQ ID Nos. 1-36 or 74-91.

6. The host cell of claim 1, which is an eukaryotic cell or a prokaryotic cell.

7. The host cell of claim 5, wherein the vector or nucleic acid is maintained extrachromosomally.

8. The host cell of 6 which is an insect cell, a plant cell, or a mammalian cell.

9. A method to increase influenza virus yield in cells, comprising: contacting influenza virus and cells comprising the vector of any one of claim 1 or contacting the cells with a polypeptide having at least 80% amino acid sequence identity to one of SEQ ID Nos. 1-36 or 74-91 or a portion thereof with the activity of SEQ ID Nos. 1-36 or 74-91; and collecting progeny influenza virus.

10. The method of claim 9, wherein the cells are human, canine, or non-human primate cells.

11. The method of claim 9, wherein the cells are Vero cells, MDCK cells, 293T or PER.C6® cells, or MvLu1 cells.

12. The method of claim 9, wherein the cell is contacted with the vector or the polypeptide before contacting the cell with the influenza virus.

13. The method of claim 9, wherein the cell is contacted with the vector or the polypeptide after contacting the cell with the influenza virus.

14. The method of claim 9, wherein the yield of influenza virus is increased at least two-fold relative to the corresponding yield in cells not having the vector or the polypeptide.

15. A method to detect influenza virus in a sample, comprising: contacting cells having the vector of claim 1 or contacting the cells with a polypeptide having at least 80% amino acid sequence identity to one of SEQ ID Nos. 1-36 or 74-91 or a portion thereof with the activity of SEQ ID Nos. 1-36 or 74-91 and a biological sample; and determining whether the sample comprises influenza virus.

16. The method of claim 15, wherein the cells are human, canine or non-human primate cells.

17. The method of claim 15, wherein the cells are Vero cells, MDCK cells, 293T or PER.C6® cells, or MvLu1 cells.

18. The method of claim 15, wherein the sample is a physiological sample.

19. The method of claim 18, wherein the sample is a nasal sample.

20. A method to decrease influenza virus replication in a mammal, comprising: administering to the mammal a composition comprising the vector of claim 1 or a polypeptide having at least 80% amino acid sequence identity to one of SEQ ID Nos. 1-36 or 74-91 or a portion thereof with the activity of SEQ ID Nos. 1-36 or 74-91.

21. A method to screen for compounds that alter the activity of a pathogen, comprising: contacting cells with a sample having a pathogen, wherein the cells express a polypeptide having at least 80% amino acid sequence identity to one of SEQ ID Nos. 1-36 or 74-91 or a portion thereof with the activity of SEQ ID Nos. 1-36 or 74-91, or wherein the cells comprise an isolated polypeptide having at least 80% amino acid sequence identity to one of SEQ ID Nos. 1-36 or 74-91 or a portion thereof with the activity of SEQ ID Nos. 1-36 or 74-91; and determining whether the polypeptide alters the activity of the pathogen.

22. The method of claim 21, wherein the pathogen is a virus.

23. The method of claim 21, wherein the cells are mammalian cells.

24. The method of claim 23, wherein the cells are canine, non-human primate, or human cells.

25. The method of claim 23, wherein the cells are MDCK cells.

26. A method to inhibit expression of pro-viral genes in a mammal, comprising administering to the mammal an effective amount a composition that specifically inhibits the expression of an amino acid sequence any one of SEQ ID Nos. 1-36 or 74-91.

27. The method of claim 26, wherein the composition comprises RNA.

28. The method of claim 27, wherein the RNA triggers RNA interference (RNAi).

29. The method of claim 28, wherein the RNA comprises a small interfering RNA (siRNA).

30. The method of claim 26, wherein the mammal is infected with influenza virus.

31. The method of claim 30, wherein the composition prevents or inhibits influenza virus replication.

32. A method to screen for inhibitory compounds, comprising combining cells expressing a polypeptide having at least 80% amino acid sequence identity to one of SEQ ID Nos. 1-36 or 74-91 or a portion thereof with the activity of SEQ ID Nos. 1-36 or 74-91 or isolated nucleic acid that encodes a polypeptide having at least 80% amino acid sequence identity to one of SEQ ID Nos. 1-36 or 74-91 or a portion thereof with the activity of SEQ ID Nos. 1-36 or 74-91 and one or more test compounds; and determining whether the one or more test compounds inhibit expression of the polypeptide or inhibit transcription or translation of the isolated nucleic acid.

33. A method to prevent, inhibit, or treat influenza virus infection in an avian or a mammal, comprising administering to the avian or mammal an effective amount of RNA that triggers RNA interference (RNAi) specific an amino acid sequence of any one of SEQ ID Nos. 1-36 or 74-91 or an antibody or fragment thereof specific for one of SEQ ID Nos. 1-36 or 74-91.

34. The method of claim 33, wherein the mammal is a human.

35. The method of claim 33, wherein the RNA comprises a small interfering RNA (siRNA).

36. The method of claim 33, wherein the composition is administered locally, systemically, or intranasally.

37. The method of claim 33, wherein the composition comprises liposomes or nanoparticles comprising the RNAi.
